(19) United States
(12) Patent Application Publication
(10) Pub. No.: US 2025/0154163 A1
(43) Pub. Date: May 15, 2025

(54) Pyrimidinone-Containing 17-Beta-Hydroxysteroid Dehydrogenase Type 13 Inhibitors

(71) Applicant: ENANTA PHARMACEUTICALS, INC., Watertown, MA (US)

(72) Inventors: Yat Sun OR, Waltham, MA (US); Joseph D. PANARESE, Newton, MA (US); Guoqiang WANG, Belmont, MA (US); Jing HE, Somerville, MA (US); Jiang LONG, Wayland, MA (US); Jun MA, Wayland, MA (US); Bin WANG, Newton, MA (US); Sourav GHORAI, Waltham, MA (US); Scott MITCHELL, Woburn, MA (US); Tao WANG, Berkeley Heights, NJ (US); Weipeng HU, Brighton, MA (US); Han Seul PARK, Watertown, MA (US); Xin ZHANG, Waltham, MA (US); Hui CAO, Belmont, MA (US)

(21) Appl. No.: 18/926,885

(22) Filed: Oct. 25, 2024

Related U.S. Application Data
(60) Provisional application No. 63/545,800, filed on Oct. 26, 2023.

(51) Int. Cl. C07D 495/04; A61K 31/519; A61K 31/7064; A61P 1/16; A61P 11/00; C07D 495/14; C07D 498/14; C07H 17/02

(52) U.S. Cl. CPC C07D 495/04; A61K 31/519; A61K 31/7064; A61P 1/16; A61P 11/00; C07D 495/14; C07D 498/14; C07H 17/02

(57) ABSTRACT

The present invention provides compounds of Formula (I),

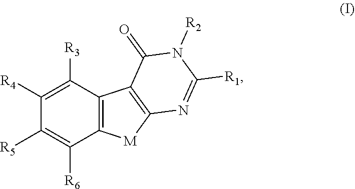

pharmaceutical compositions comprising these compounds and methods of using these compounds for treating a metabolic disease or liver condition. The present invention relates generally to compounds and pharmaceutical compositions useful as 17β-HSD13 inhibitors. Specifically, the present invention relates to compounds useful as inhibitors of 17β-HSD13 and methods for their preparation and use.

### RELATED APPLICATION

This application claims the benefit of U.S. Provisional Application No. 63/545,800, filed on Oct. 26, 2023. The entire teachings of the above application are incorporated herein by reference.

### TECHNICAL FIELD

The present invention relates generally to compounds and pharmaceutical compositions useful as 17β-HSD13 inhibitors. Specifically, the present invention relates to compounds useful as inhibitors of 17β-HSD13 and methods for their preparation and use.

### BACKGROUND OF THE INVENTION

17-Beta-hydroxysteroid dehydrogenases (17β-HSDs) are NADP or NAD dependent oxidoreductases that catalyze oxidation/reduction reactions of 17β-hydroxysteroids or 17-ketosteroids, respectively. For example, 17β-HSDs can catalyze the interconversion of androstenedione with testosterone, estrone with estradiol, or dehydroepiandrosterone (DHEA) with androstenediol. Of the fifteen 17β-HSDs that have been identified, all but one (17β-HSD type 5) are short-chain dehydrogenases/reductases (SDRs) (J. M. Day, et al., Endocrine-Related Cancer 2008, 15, 665-692).

More specifically, 17-Beta-hydroxysteroid dehydrogenase type 13 (17β-HSD13) is encoded by the HSD17B13 gene and is mainly expressed in the liver (S. Liu, et al., Acta Biochim. Pol. 2007, 54, 213-218). Moreover, 17β-HSD13 was identified as a lipid droplet associated protein and is up-regulated in mice and patients with nonalcoholic fatty liver disease (NAFLD) (Y. Horiguchi, et al., Biochem. Biophys. Res. Commun. 2008, 370, 235-238; W. Su, et al., Mol. Cell. Endocrinol. 2019, 489, 119-125). Further studies have shown that a 17β-HSD13 loss-of-function variant has been associated with a significantly reduced risk of NAFLD, cirrhosis associated with nonalcoholic steatohepatitis (NASH), alcoholic liver disease, alcoholic cirrhosis, hepatocellular carcinoma (HCC), NASH disease severity, ballooning degeneration, lobular inflammation, and fibrosis (N. S. Abul-Husn, et al., N. Engl. J. Med. 2018, 378, 1096-1106; C. J. Pirola, et al., J. Lipid Res. 2019, 60, 176-185). This variant has also shown a reduction in liver damage among obese children (A. Di Sessa, et al., J. Pediatr. Gastroenterol. Nutr. 2020, 70, 371-374).

Recently small molecule compounds which act as 17β-HSD13 inhibitors have been published, see, for example, WO 2024/173343, WO 2024/127297, WO 2024/075051, WO 2023/237504, WO 2023/223257, WO 2023/222850, WO 2023/222849, WO 2023/224981, Nature Communications (2023, 14:5158), J. Med. Chem. (2023, 66, 2832-2850), US2023/0286923, US 2023/0150940, WO 2023/192375, WO 2023/146897, WO 2023/043836, WO 2023/023310, WO 2022/103960A1, WO 2022/216626, WO 2022/216627, WO 2022/020714, WO 2022/020730, WO 2021/211974, and WO 2021/003295A1. Other agents that act as 17β-HSD13 inhibitors have been disclosed in WO 2024/164001, WO 2024/131916, WO 2024/059165, WO 2023/208109, WO 2023/220561, WO 2023/213284, WO 2023/196941, WO 2023/192314, WO 2023/138650, WO 2021/211981, WO 2021/211959, WO 2020/132564, WO 2020/061177, WO 2019/075181, WO 2019/183164, WO 2019/183329, US 2019/0106749, and WO 2018/136758.

Fibrosis can affect various organs and tissues in the body, such as the lungs (pulmonary fibrosis), liver (cirrhosis), kidney (renal fibrosis), heart (cardiac fibrosis), and skin (scleroderma), among others.

In addition to liver cirrhosis, pulmonary fibrosis is one of the most critical fibrotic diseases to treat. Pulmonary fibrosis is defined as the progressive replacement of alveolar tissue with fibrotic scar that threatens alveolar gas exchange and reduces lung compliance. The resulting increased work of breathing and hypoxemia lead to progressive respiratory failure and eventual death. There are two main types of pulmonary fibrosis: idiopathic pulmonary fibrosis (IPF) and secondary pulmonary fibrosis. Idiopathic pulmonary fibrosis refers to cases where the specific cause of the condition is unknown, whereas secondary pulmonary fibrosis is associated with known causes, such as certain occupational exposures, connective tissue diseases, medication side effects, or other underlying medical conditions. The clinical and economic burden of pulmonary fibrosis is sizeable: the worldwide incidence of IPF is increasing and attributable medical costs for patients with IPF in the United States alone have been estimated at nearly 2 billion dollars (Hutchinson J, Fogarty A, Hubbard R, McKeever T. Global incidence and mortality of idiopathic pulmonary fibrosis: a systematic review. Eur Respir J. 2015; 46:795-806). Despite decades of research, only a handful of therapies are available. Although these therapies slow disease progression, the response is variable, and lung transplantation represents the only option for temporary cure. Also, both pirfenidone and nintedanib have been shown in clinical trials to slow the decline in lung function, decrease the rate of disease progression, and improve overall quality of life for some individuals with IPF. It's important to note that not all patients respond the same way to these medications, and individual responses can vary. Accordingly, there is an urgent need for new agents useful for diagnosis and/or therapy of pulmonary fibrosis disease.

There is a need for the development of 17β-HSD13 inhibitors for the treatment and prevention of disease. The present invention has identified compounds which inhibit 17β-HSD13 as well as methods of using these compounds to treat and prevent fibrotic diseases, such as pulmonary fibrosis and liver cirrhosis associated with NASH.

### SUMMARY OF THE INVENTION

The present invention relates to compounds and pharmaceutical compositions useful as 17β-HSD13 inhibitors. Specifically, the present invention relates to compounds useful as inhibitors of 17β-HSD13 and methods for their preparation and use. In addition, the present invention includes the process for the preparation of the said compounds.

In its principal aspect, the present invention provides compounds represented by Formula (I), and pharmaceutically acceptable salts and esters thereof:

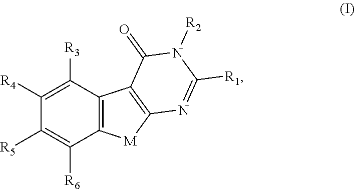

wherein,
M is S, SO, SO₂, O, or NR₇;
R₁ and R₂ are each independently selected from the group consisting of:
   1) Hydrogen;
   2) Optionally substituted —C₁-C₈ alkyl;
   3) Optionally substituted —C₂-C₈ alkenyl;
   4) Optionally substituted —C₂-C₈ alkynyl;
   5) Optionally substituted —C₃-C₈ cycloalkyl;
   6) Optionally substituted aryl;
   7) Optionally substituted arylalkyl;
   8) Optionally substituted 3- to 8-membered heterocycloalkyl;
   9) Optionally substituted heteroaryl; and
   10) Optionally substituted heteroarylalkyl;
R₃, R₄, R₅, and R₆ are each independently selected from the group consisting of hydrogen, halogen, —CN, —OR₉, —SR₉, —B(OR₁₃)₂, —SO₂R₁₃, —SO₂OR₁₃, —OSO₂OR₁₃, —P(O)(OR₁₃)₂, —C(O)R₇, —C(O)OR₇, —NR₇R₈, —NR₇COR₈, —NR₇C(O)OR₈, —N(COR₈)₂, —NR₇SO₂R₈, —C(O)NR₇R₈, optionally substituted —C₁-C₈ alkyl, optionally substituted —C₂-C₈ alkenyl, optionally substituted aryl, and optionally substituted heteroaryl;
alternatively, R₅ and R₆ are taken together with the carbon atoms to which they are attached to form an optionally substituted carbocyclic or heterocyclic ring;
alternatively, R₄ and R₅ are taken together with the carbon atoms to which they are attached to form an optionally substituted carbocyclic or heterocyclic ring;
alternatively, R₃ and R₄ are taken together with the carbon atoms to which they are attached to form an optionally substituted carbocyclic or heterocyclic ring;
each R₇ and R₈ is independently selected from the group consisting of:
   1) Hydrogen;
   2) Optionally substituted —C₁-C₈ alkyl;
   3) Optionally substituted —C₂-C₈ alkenyl;
   4) Optionally substituted —C₂-C₈ alkynyl;
   5) Optionally substituted —C₃-C₈ cycloalkyl;
   6) Optionally substituted 3- to 8-membered heterocycloalkyl;
   7) Optionally substituted aryl;
   8) Optionally substituted arylalkyl;
   9) Optionally substituted heteroaryl; and
   10) Optionally substituted heteroarylalkyl;
alternatively, R₇ and R₈ are taken together with the nitrogen atom to which they are attached to form an optionally substituted heterocyclic ring;
R₉ is selected from the group consisting of:
   1) Hydrogen;
   2) Optionally substituted —C₁-C₈ alkyl;
   3) Optionally substituted —C₂-C₈ alkenyl;
   4) Optionally substituted —C₂-C₈ alkynyl;
   5) Optionally substituted —C₃-C₈ cycloalkyl;
   6) Optionally substituted 3- to 8-membered heterocycloalkyl;
   7) Optionally substituted aryl;
   8) Optionally substituted arylalkyl;
   9) Optionally substituted heteroaryl;
   10) Optionally substituted heteroarylalkyl;
   11) —C(O)R₁₁;
   12) —C(O)NR₁₁R₁₂;
   13) —C(O)OR₁₁;
   14) —P(O)(OR₁₃)₂; and
   15) —P(O)(OR₁₃)(NR₁₁R₁₂);
R₁₁ and R₁₂ are each independently selected from the group consisting of:
   1) Hydrogen;
   2) Optionally substituted —C₁-C₈ alkyl;
   3) Optionally substituted —C₂-C₈ alkenyl;
   4) Optionally substituted —C₂-C₈ alkynyl;
   5) Optionally substituted —C₃-C₈ cycloalkyl;
   6) Optionally substituted 3- to 8-membered heterocycloalkyl;
   7) Optionally substituted aryl;
   8) Optionally substituted arylalkyl;
   9) Optionally substituted heteroaryl; and
   10) Optionally substituted heteroarylalkyl;
R₁₃ is hydrogen, optionally substituted —C₁-C₈ alkyl, or Na⁺.

In certain embodiments, the present invention provides a pharmaceutical composition comprising a therapeutically effective amount of a compound or combination of compounds of the present invention, or a pharmaceutically acceptable salt, ester or combination thereof, in combination with a pharmaceutically acceptable carrier or excipient.

In certain embodiments, the present invention provides a method for the prevention or treatment of an 17β-HSD13 mediated disease or condition. The method comprises administering a therapeutically effective amount of a compound of Formula (I) or a pharmaceutically acceptable salt thereof. The present invention also provides the use of a compound of Formula (I), or a pharmaceutically acceptable salt thereof, in the manufacture of a medicament for the prevention or treatment of a 17β-HSD13 mediated disease or condition including, but not limited to: nonalcoholic fatty liver disease (NAFLD), nonalcoholic steatohepatitis (NASH), liver cirrhosis, liver fibrosis, hepatocellular carcinoma (HCC), other metabolic disorders, and fibrotic diseases, such as pulmonary fibrosis.

### BRIEF DESCRIPTION OF THE DRAWINGS

FIG. 1 presents graphs showing the effects of Compound 26 on (a) liver collagen (Col1a1) mRNA, (b) the amount of liver TGFB2, and (c) the amount of liver fibronectin in a mouse model of chronic liver injury (choline deficient, L-amino acid defined, high fat diet; A16092201).

FIG. 2 presents graphs showing the effects of Compound 26 on (a) collagen (COL1A1) mRNA in human precision cut lung slices from a normal donor with and without fibrotic cocktail treatment and (b) on lung fibronectin content in human precision cut lung slices from a donor with idiopathic pulmonary fibrosis.

FIG. 3 is a graph showing the effect of Compound 64 on collagen (COL1A1) mRNA in human precision cut lung slices from a normal donor with and without fibrotic cocktail treatment.

### DETAILED DESCRIPTION OF THE INVENTION

In one embodiment of the invention is a compound represented by Formula (I) as described above, or a pharmaceutically acceptable salt or ester thereof.

In certain embodiments of the compounds of Formula (I), M is S.

In certain embodiments of the compounds of Formula (I), R₃ is hydrogen or halogen.

In certain embodiments of the compounds of Formula (I), R₄ is hydrogen or halogen.

In certain embodiments of the compounds of Formula (I), R₅ is hydrogen or halogen.

In certain embodiments of the compounds of Formula (I), R₅ is halogen.

In certain embodiments of the compounds of Formula (I), R₅ is —Cl or —F.

In certain embodiments of the compounds of Formula (I), R₃ is hydrogen, and R₄ is hydrogen.

In certain embodiments of the compounds of Formula (I), R₆ is —OR₉.

In certain embodiments of the compounds of Formula (I), R₆ is —OH.

In certain embodiments of the compounds of Formula (I), R₆ is —B(OR₁₃)₂.

In certain embodiments of the compounds of Formula (I), R₁ is optionally substituted aryl or optionally substituted heteroaryl.

In certain embodiments of the compounds of Formula (I), R₁ is optionally substituted heterocycloalkyl-C₁-C₆-alkyl.

In certain embodiments of the compounds of Formula (I), R₁ is optionally substituted —C₃-C₈ cycloalkyl or optionally substituted 3- to 8-membered heterocycloalkyl.

In certain embodiments of the compounds of Formula (I), R₁ is selected from the groups below, wherein each group is optionally substituted:

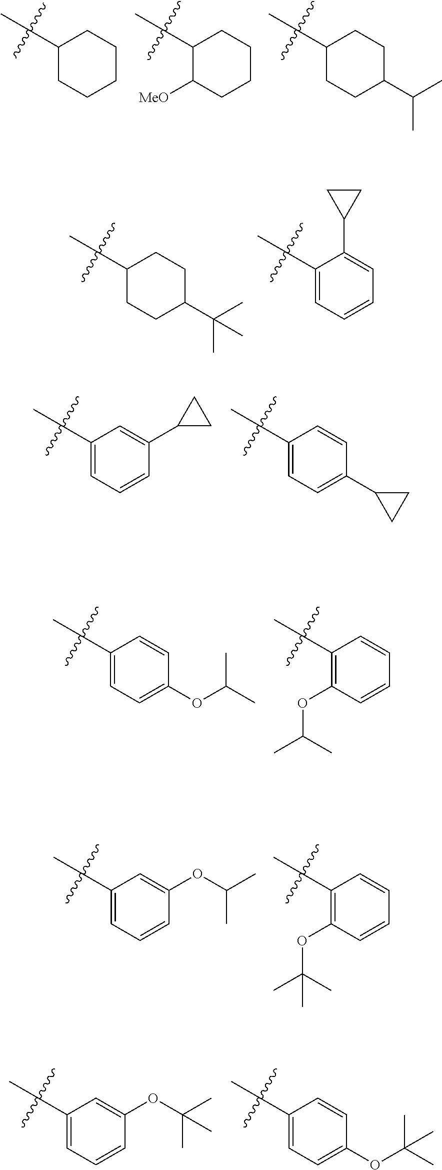

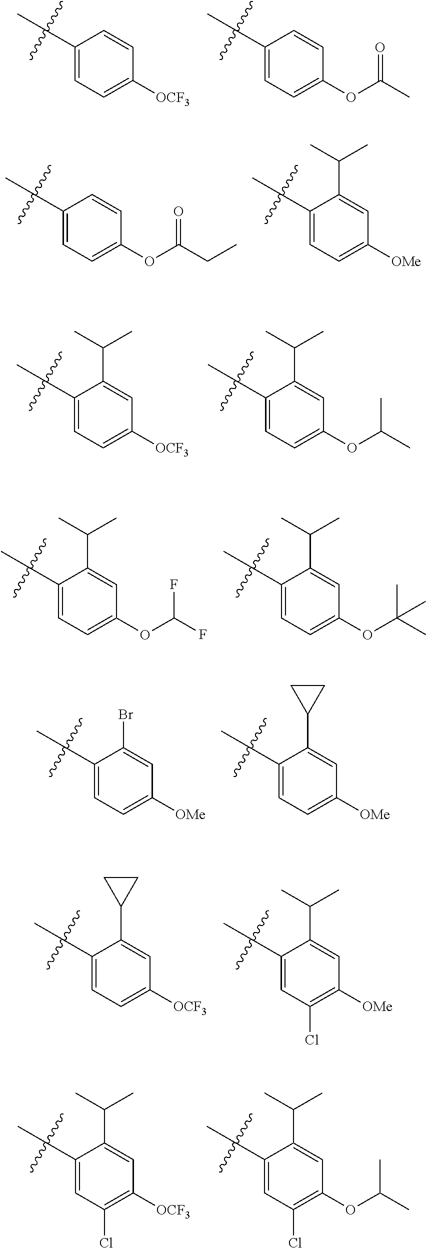

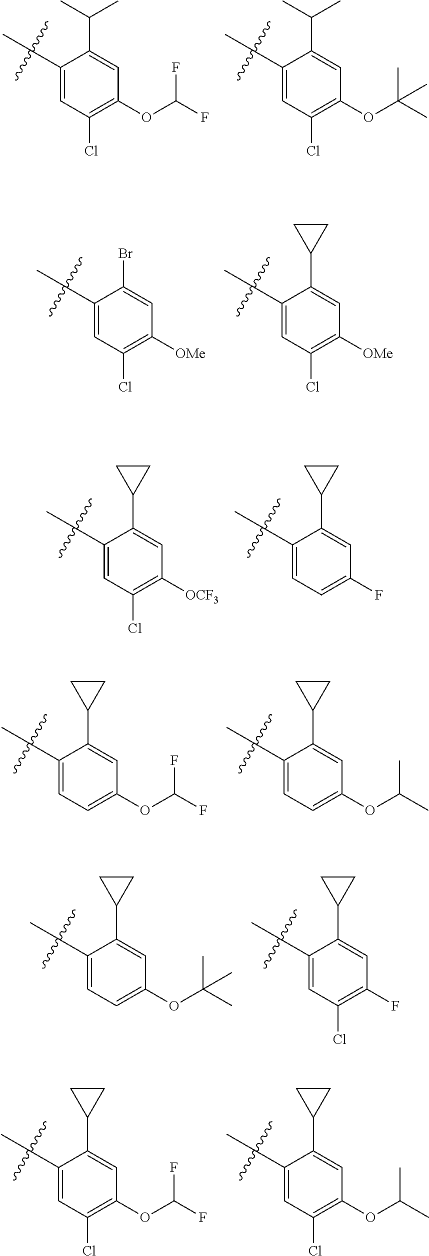

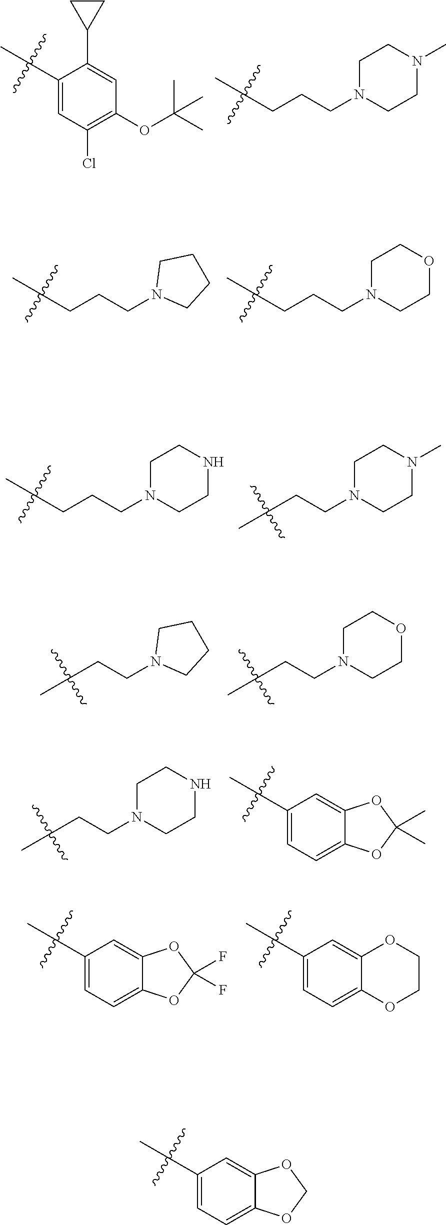

In certain embodiments of the compounds of Formula (I), R₂ is optionally substituted —C₁-C₈ alkyl, preferably optionally substituted —C₁-C₆-alkyl. Preferred substituents include halogen, C₃-C₆-cycloalkyl, hydroxy, amino, C₁-C₆-alkyl amino, di(C₁-C₆-alkyl)amino, C₁-C₆-alkylNHC(O)—, di(C₁-C₆-alkyl)NC(O)— and —C(O)O—C₁-C₆-alkyl.

In certain embodiments of the compounds of Formula (I), R₂ is optionally substituted —C₂-C₅ alkenyl, preferably optionally substituted C₂-C₆-alkenyl. Preferred substituents include halogen, C₃-C₆-cycloalkyl, hydroxy, amino, C₁-C₆-alkyl amino, di(C₁-C₆-alkyl)amino and —C(O)O—C₁-C₆-alkyl.

In certain embodiments of the compounds of Formula (I), R₂ is optionally substituted —C₃-C₈ cycloalkyl, preferably optionally substituted C₃-C₆-cycloalkyl. Preferred substituents include halogen, C₁-C₄-alkyl, and hydroxy.

In certain embodiments of the compounds of Formula (I), R₂ is optionally substituted arylalkyl or optionally substituted heteroarylalkyl.

In certain embodiments of the compounds of Formula (I), R₂ is optionally substituted aryl-C₁-C₆-alkyl, optionally substituted heteroaryl-C₁-C₆-alkyl or optionally substituted heterocyclyl-C₁-C₆-alkyl, preferably optionally substituted aryl-C₁-C₄-alkyl, optionally substituted heteroaryl-C₁-C₄-alkyl or optionally substituted heterocyclyl-C₁-C₄-alkyl. Preferred substituents include halogen, C₁-C₄-alkyl, and hydroxy.

In certain embodiments of the compounds of Formula (I), R₂ is optionally substituted aryl-C₂-C₆-alkenyl, optionally substituted heteroaryl-C₂-C₆-alkenyl or optionally substituted heterocyclyl-C₂-C₆-alkenyl, preferably optionally substituted aryl-C₂-C₄-alkenyl, optionally substituted heteroaryl-C₂-C₄-alkenyl or optionally substituted heterocyclyl-C₂-C₄-alkenyl. Preferred substituents include halogen, C₁-C₄-alkyl, and hydroxy.

In certain embodiments of the compounds of Formula (I), R₂ is optionally substituted —C₁-C₄-alkylN(R)—C₁-C₄-alkylaryl, optionally substituted —C₁-C₄-alkylN(R)—C₁-C₄-alkylheteroaryl or optionally substituted —C₁-C₄-alkylN(R)—C₁-C₄-alkylheterocyclyl, where R is H or C₁-C₄-alkyl. Preferred substituents include halogen, C₁-C₄-alkyl, and hydroxy.

In certain embodiments of the compounds of Formula (I), R₂ is —C₁-C₆-alkyl-L-R′, where L is —O—, —S—, —N(R)—, —N(R)C(O)—, —NRC(O)O—, or —N(R)SO₂—; R′ is optionally substituted C₁-C₆-alkyl, optionally substituted aryl, or optionally substituted heteroaryl; alternatively R, R′ and the nitrogen atom to which they are attached form an optionally substituted 3- to 8-membered heterocyclyl.

In certain embodiments of the compounds of Formula (I), R₂ is selected from the group below, wherein each is optionally substituted:

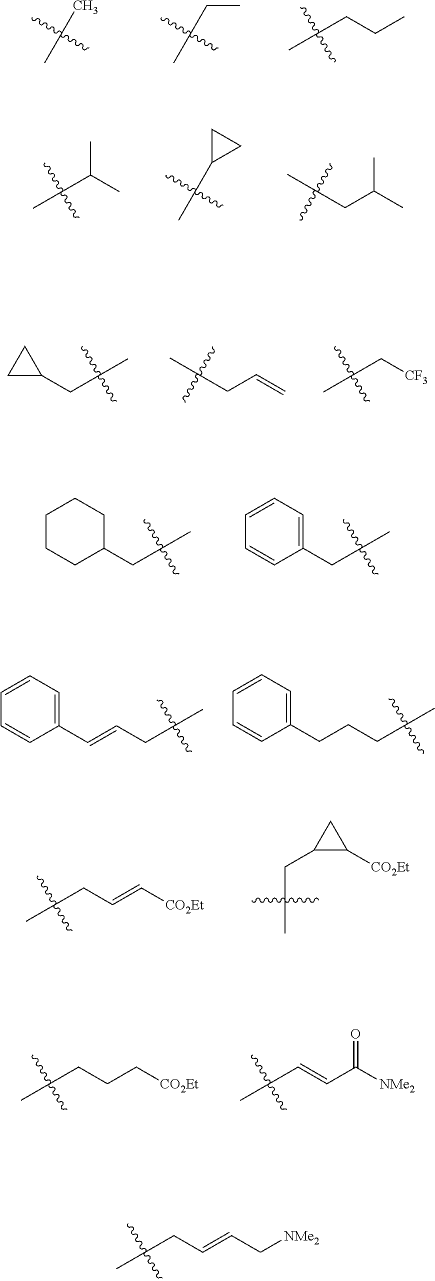

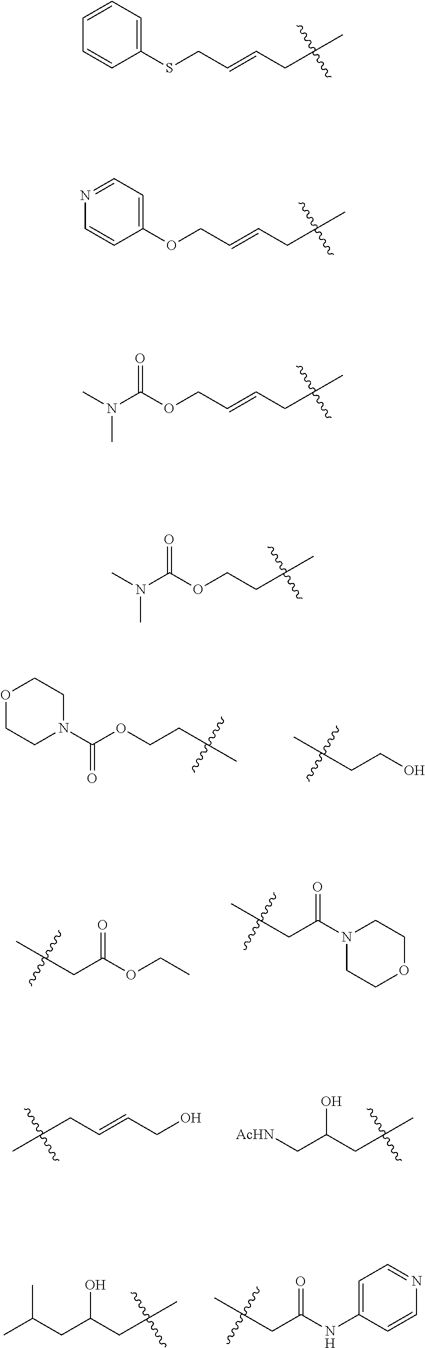

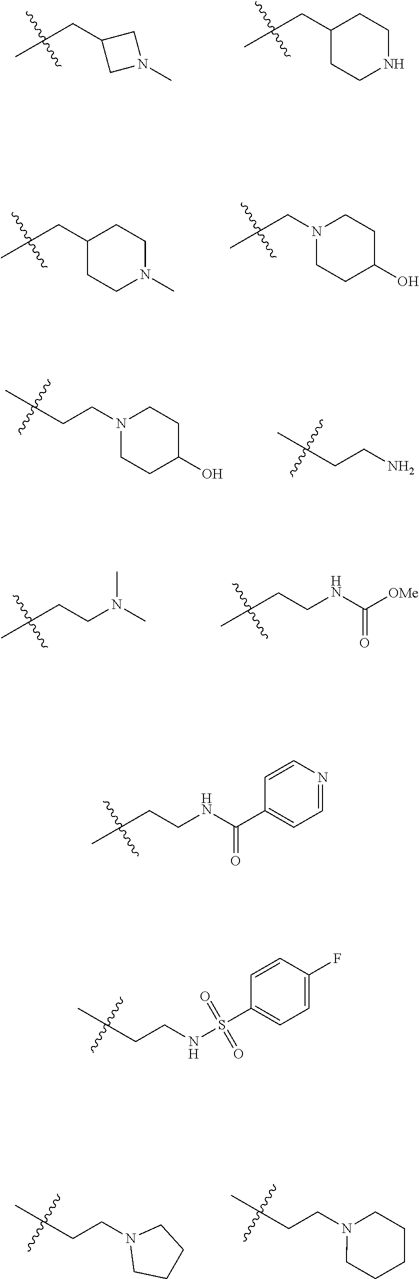

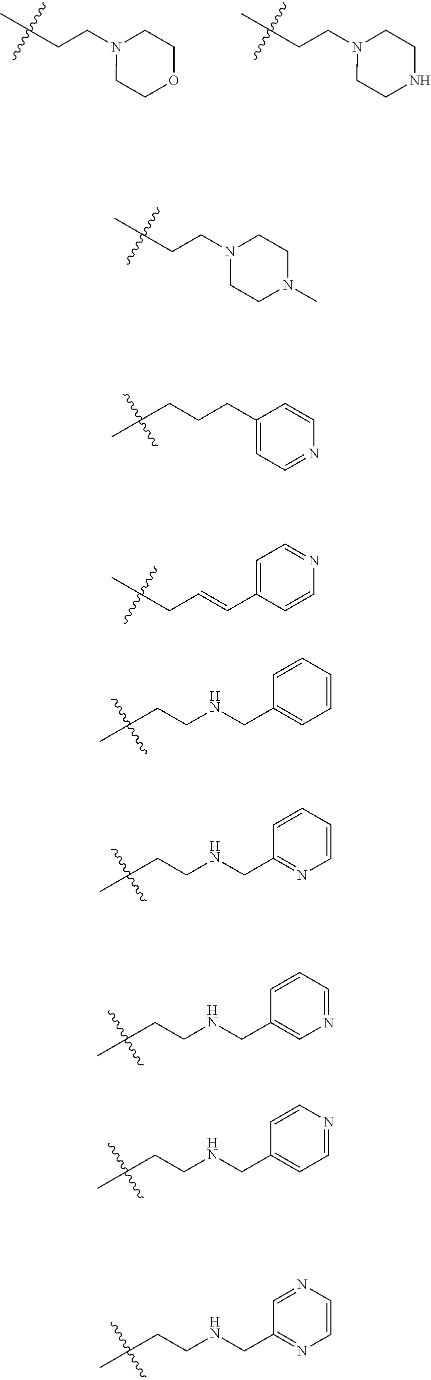

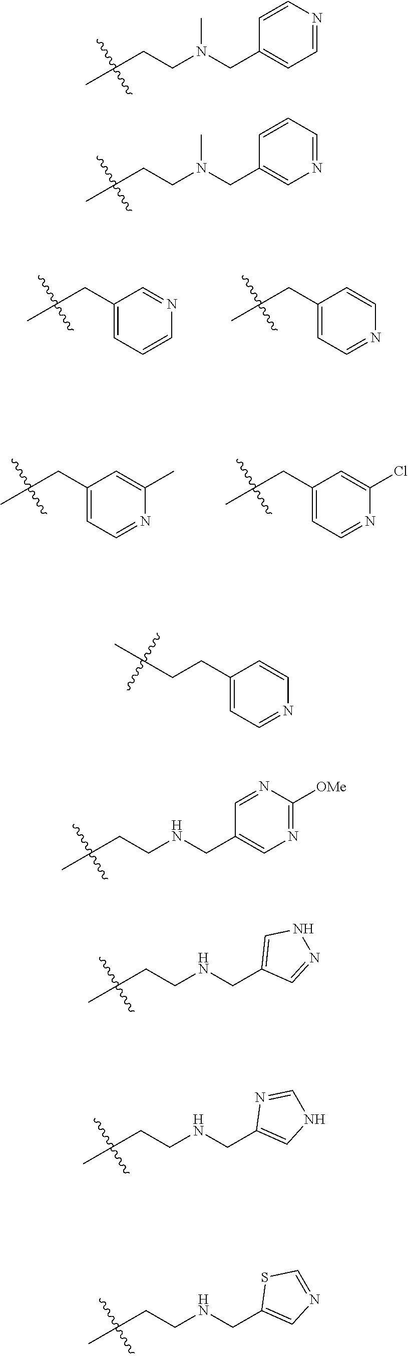

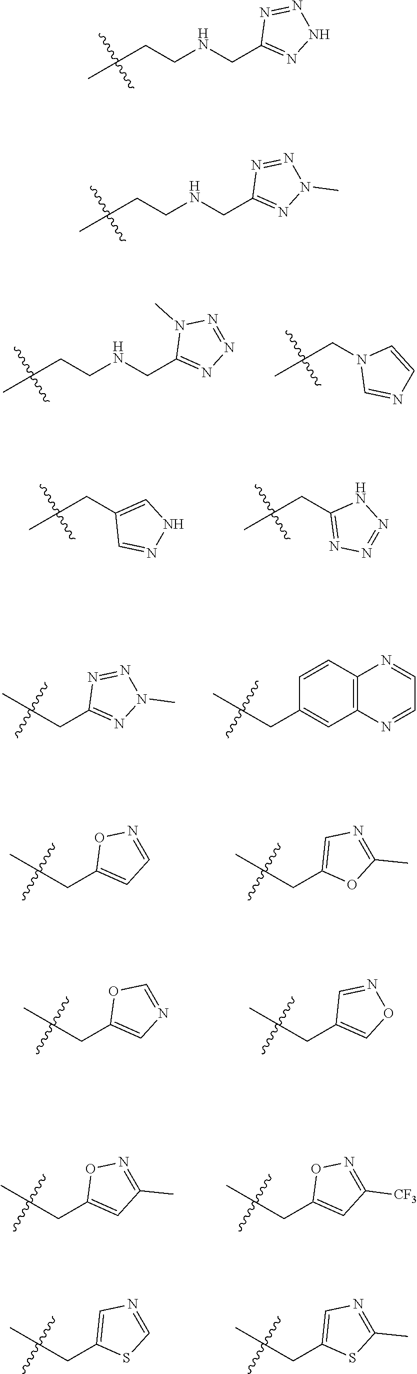

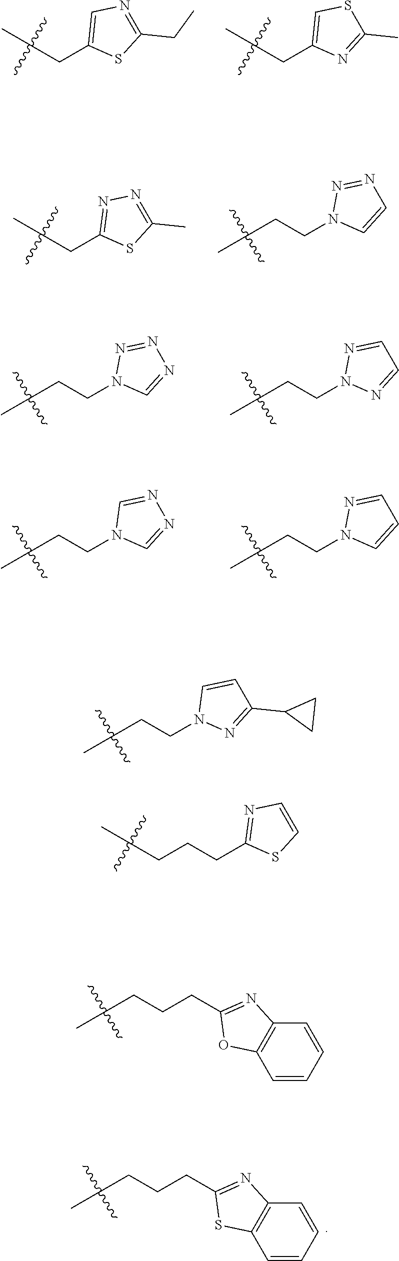

In certain embodiments of the compounds of Formula (I), at least one of R₃, R₄, R₅, and R₆ is —B(OR₁₃)₂, —SO₂R₁₃, —SO₂₀R₁₃, —OSO₂OR₁₃, —P(O)(OR₁₃)₂, —C(O)R₇, —C(O)OR₇, —NR₇R₈, —NR₇(COR₈), —NR₇C(O)OR₈, —N(COR₈)₂, or —NR₇SO₂R₈. Preferably only one of R₃, R₄, R₅, and R₆ is —B(OR₁₃)₂, —SO₂R₁₃, —SO₂₀R₁₃, —OSO₂OR₁₃, —P(O)(OR₁₃)₂, —C(O)R₇, —C(O)OR₇, —NR₇R₈, —NR₇(COR₈), —NR₇C(O)OR₈, —N(COR₈)₂, or —NR₇SO₂R₈. In particular preferred embodiments, R₆ is —B(OR₁₃)₂, —SO₂R₁₃, —SO₂₀R₁₃, —OSO₂OR₁₃, —P(O)(OR₁₃)₂, —C(O)R₇, —C(O)OR₇, —NR₇R₈, —NR₇(COR₈), —NR₇C(O)OR₈, —N(COR₈)₂, or —NR₇SO₂R₈.

In certain embodiments of the compounds of Formula (I), R₅ and R₆ are not hydrogen. In certain embodiments R₃, R₅, and R₆ are not hydrogen. In certain embodiments, R₃ and R₄ are hydrogen and R₅ and R₆ are not hydrogen. In certain embodiments of the compounds of Formula (I), R₄ is hydrogen and R₃, R₅, and R₆ are not hydrogen.

In certain embodiments of the compounds of Formula (I), R₅ is halogen and R₆ is hydroxyl. In these embodiments, R₃ and R₄ are preferably hydrogen.

In certain embodiments of the compounds of Formula (I), R₂ is

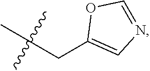 [SMILES: *Cc1cnco1]

M is sulfur and R₁ is

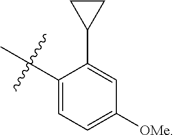 [SMILES: *c1ccc(OC)cc1C1CC1]

In certain embodiments of the compounds of Formula (I), R₅ is halogen, R₆ is hydroxyl, R₃ and R₄ are hydrogen and R₂ is 5-membered heteroarylmethyl, preferably

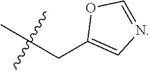 [SMILES: *Cc1cnco1]

In certain embodiments of the compounds of Formula (I), R₅ is halogen, R₆ is hydroxyl, R₃ and R₄ are hydrogen, R₂ is

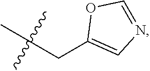 [SMILES: *Cc1cnco1]

M is sulfur and R₁ is

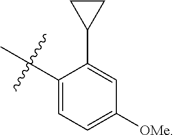 [SMILES: *c1ccc(OC)cc1C1CC1]

In one embodiment, the present invention provides compounds represented by Formula (II) or (III), or a pharmaceutically acceptable salt or ester thereof:

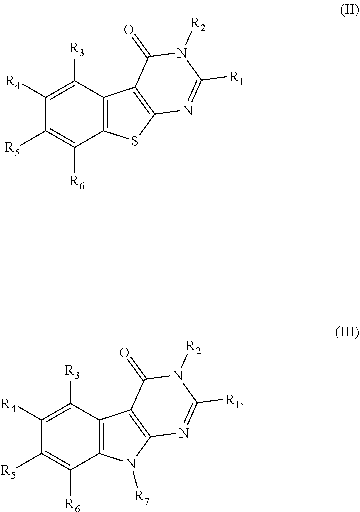

wherein, R₁, R₂, R₃, R₄, R₅, R₆, and R₇ are as previously defined.

In certain embodiments, the present invention provides compounds represented by Formula (IV) or (V), or a pharmaceutically acceptable salt or ester thereof:

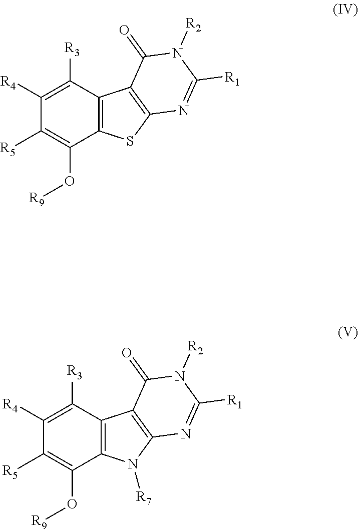

wherein, R₁, R₂, R₃, R₄, R₅, R₇, and R₉ are as previously defined.

In certain embodiments, the present invention provides compounds represented by Formula (IV) or (V), or a pharmaceutically acceptable salt or ester thereof, wherein R₉ is hydrogen or selected from the groups below, wherein each group is optionally substituted:

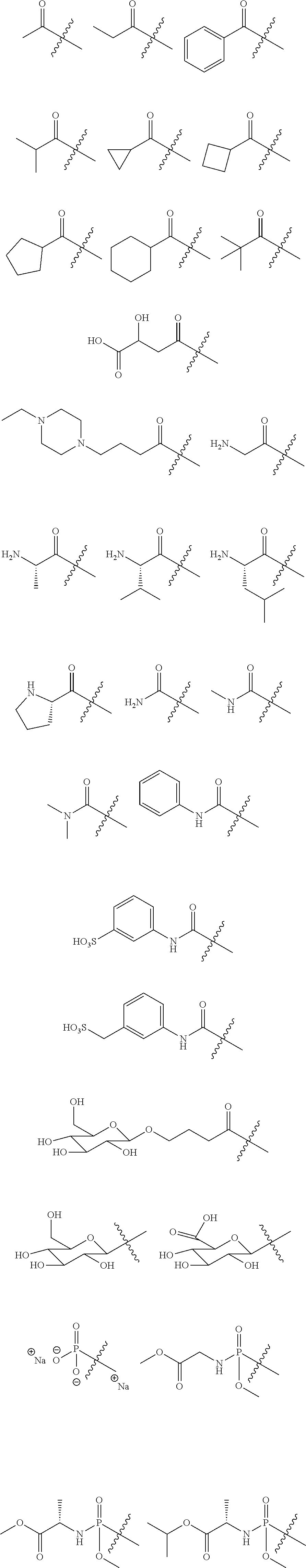

In one embodiment, the present invention provides compounds represented by Formula (VI) or (VII), or a pharmaceutically acceptable salt or ester thereof:

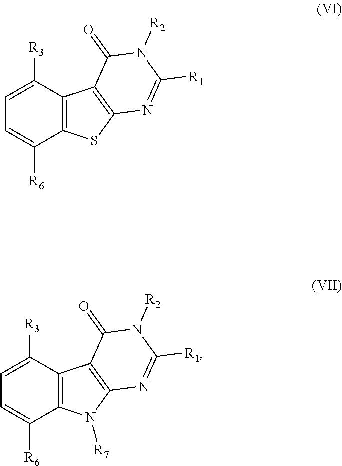

wherein, R₁, R₂, R₃, R₆, and R₇ are as previously defined, and R₆ is not hydrogen.

In certain embodiments, the present invention provides compounds represented by Formula (VIII) or (IX), or a pharmaceutically acceptable salt or ester thereof:

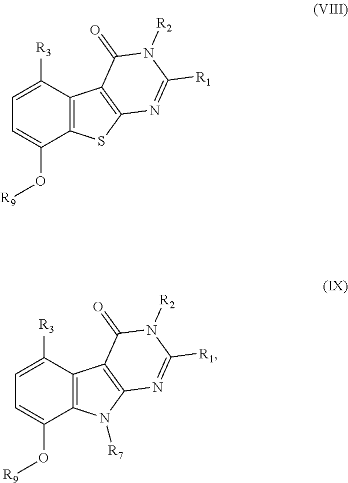

wherein, R₁, R₂, R₃, R₇, and R₉ are as previously defend, an R₃ is not hydrogen.

In certain embodiments, the present invention provides compounds represented by Formula (X) or (XI), or a pharmaceutically acceptable salt or ester thereof:

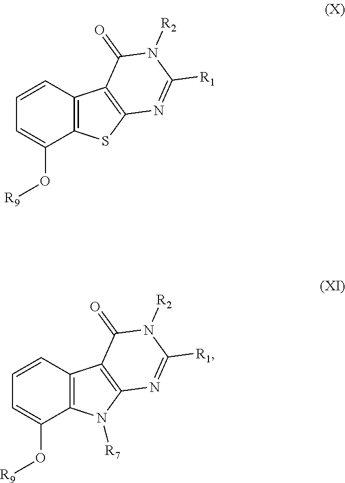

wherein, R₁, R₂, R₇, and R₉ are as previously defined. Preferably R₉ is hydrogen.

In certain embodiments, the present invention provides compounds represented by Formula (VIII), (IX), (X) or (XI), or a pharmaceutically acceptable salt or ester thereof, wherein R₉ is selected from the group consisting of below, wherein each of them is optionally substituted:

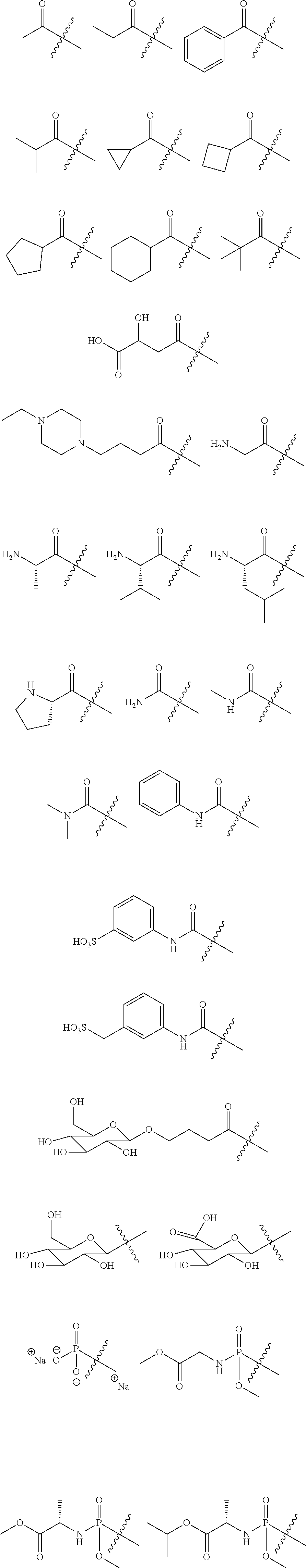

In one embodiment, the present invention provides compounds represented by Formula (XII) or (XIII), or a pharmaceutically acceptable salt or ester thereof:

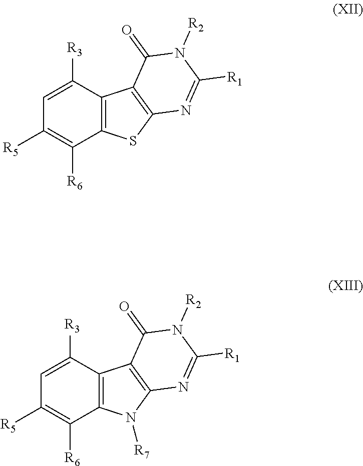

wherein, R₁, R₂, R₃, R₅, R₆, and R₇ are as previously defined, provided that R₅ and R₆ are not hydrogen.

In certain embodiments, the present invention provides compounds represented by Formula (XIV) or (XV), or a pharmaceutically acceptable salt or ester thereof:

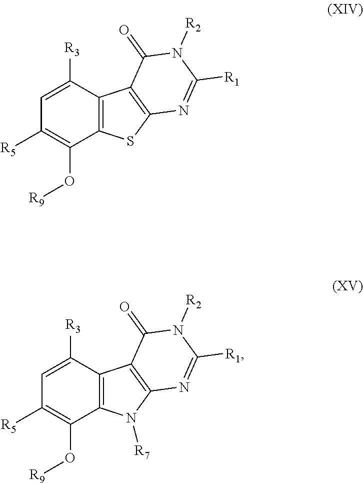

wherein, R₁, R₂, R₃, R₅, R₇, and R₉ are as previously defined, provided that R₃ and R₅ are not hydrogen.

In certain embodiments, the present invention provides compounds represented by Formula (XVI) or (XVII), or a pharmaceutically acceptable salt or ester thereof:

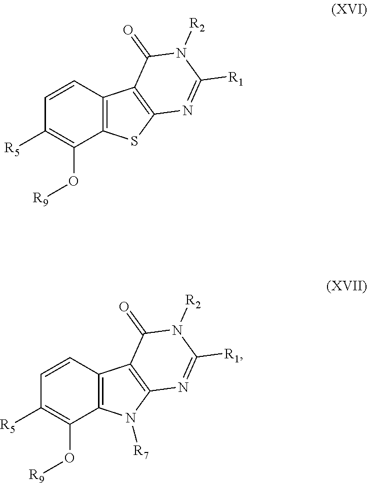

wherein, R₁, R₂, R₅, R₇, and R₉ are as previously defined, and R₅ is not hydrogen. In certain embodiments, R₅ is halogen, preferably fluorine or chlorine and more preferably chlorine. R₉ is preferably hydrogen.

In certain embodiments, the present invention provides compounds represented by Formula (XIV), (XV), (XVI) or (XVII), or a pharmaceutically acceptable salt or ester thereof, wherein R₉ is hydrogen or selected from the group consisting of below, wherein each of them is optionally substituted:

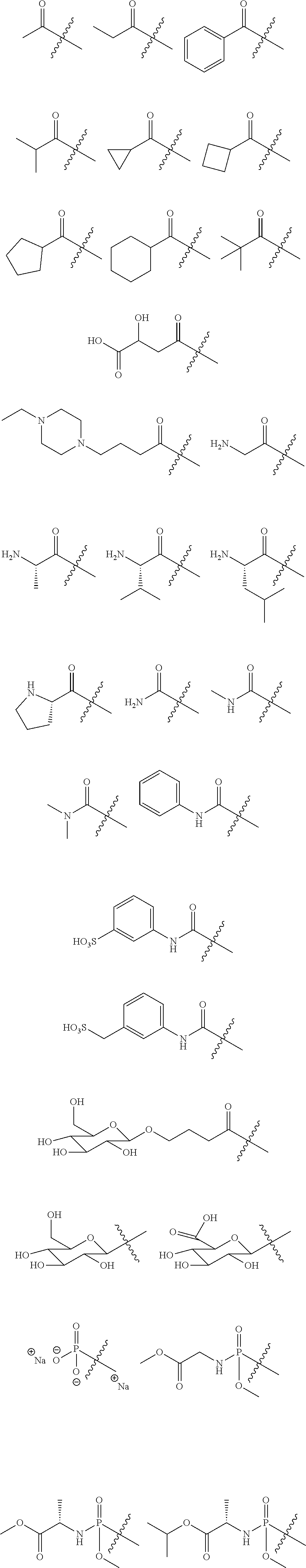

Representative compounds of the invention include, but are not limited to, the following compounds (Entry 1 to Entry 80 in Table 2) according to Formula (XVI),

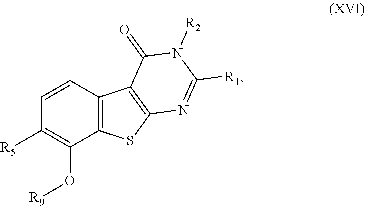

wherein R₉ is hydrogen, R₅ is not hydrogen, and R₁ and R₂ are delineated for each compound in Table 2. In certain embodiments, R₅ is halogen, such as fluorine or chlorine, methyl, fluoromethyl, difluoromethyl, trifluoromethyl or cyclopropyl, preferably chlorine or fluorine, and more preferably chlorine.

TABLE 2

| Entry | R₁ | R₂ |
|---|---|---|
| 1 | 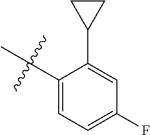 [SMILES: *c1ccc(F)cc1C1CC1] | 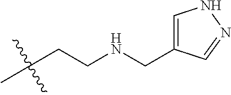 [SMILES: *CCNCc1cn[nH]c1] |
| 2 | 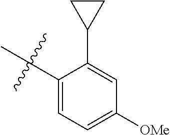 [SMILES: *c1ccc(OC)cc1C1CC1] | 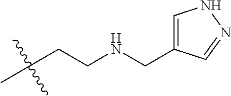 [SMILES: *CCNCc1cn[nH]c1] |
| 3 | 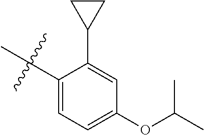 [SMILES: *c1ccc(OC(C)C)cc1C1CC1] | 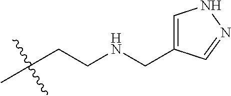 [SMILES: *CCNCc1cn[nH]c1] |
| 4 | 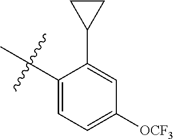 [SMILES: *c1ccc(OC(F)(F)F)cc1C1CC1] | 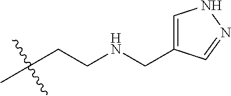 [SMILES: *CCNCc1cn[nH]c1] |
| 5 | 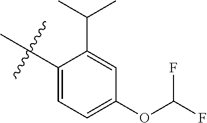 [SMILES: *c1ccc(OC(F)F)cc1C(C)C] | 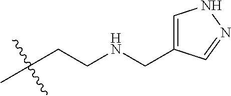 [SMILES: *CCNCc1cn[nH]c1] |
| 6 | 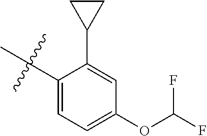 [SMILES: *c1ccc(OC(F)F)cc1C1CC1] | 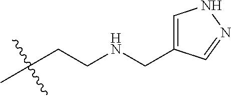 [SMILES: *CCNCc1cn[nH]c1] |
| 7 | 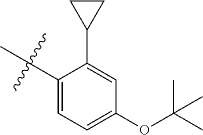 [SMILES: *c1ccc(OC(C)(C)C)cc1C1CC1] | 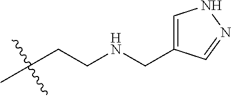 [SMILES: *CCNCc1cn[nH]c1] |
| 8 | 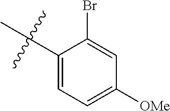 [SMILES: *c1ccc(OC)cc1Br] | 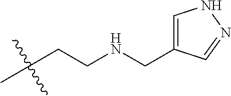 [SMILES: *CCNCc1cn[nH]c1] |
| 9 | 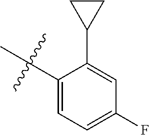 [SMILES: *c1ccc(F)cc1C1CC1] | 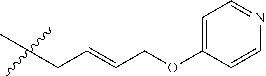 [SMILES: *C/C=C/COc1ccncc1] |
| 10 | 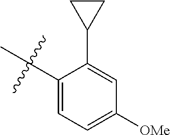 [SMILES: *c1ccc(OC)cc1C1CC1] | 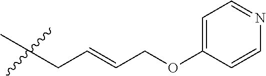 [SMILES: *C/C=C/COc1ccncc1] |
| 11 | 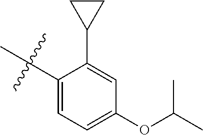 [SMILES: *c1ccc(OC(C)C)cc1C1CC1] | 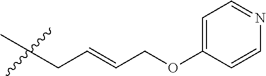 [SMILES: *C/C=C/COc1ccncc1] |
| 12 | 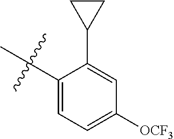 [SMILES: *c1ccc(OC(F)(F)F)cc1C1CC1] | 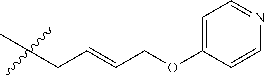 [SMILES: *C/C=C/COc1ccncc1] |
| 13 | 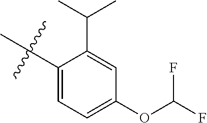 [SMILES: *c1ccc(OC(F)F)cc1C(C)C] | 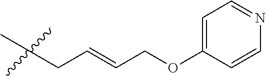 [SMILES: *C/C=C/COc1ccncc1] |
| 14 | 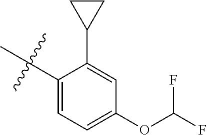 [SMILES: *c1ccc(OC(F)F)cc1C1CC1] | 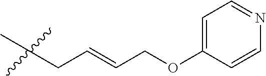 [SMILES: *C/C=C/COc1ccncc1] |
| 15 | 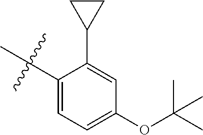 [SMILES: *c1ccc(OC(C)(C)C)cc1C1CC1] | 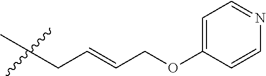 [SMILES: *C/C=C/COc1ccncc1] |
| 16 | 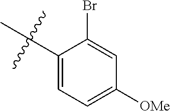 [SMILES: *c1ccc(OC)cc1Br] | 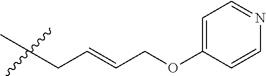 [SMILES: *C/C=C/COc1ccncc1] |
| 17 | 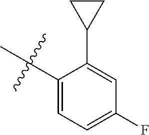 [SMILES: *c1ccc(F)cc1C1CC1] | 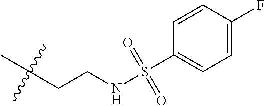 [SMILES: *CCNS(=O)(=O)c1ccc(F)cc1] |
| 18 | 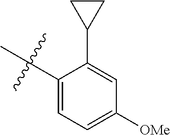 [SMILES: *c1ccc(OC)cc1C1CC1] | 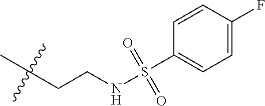 [SMILES: *CCNS(=O)(=O)c1ccc(F)cc1] |
| 19 | 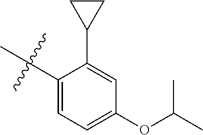 [SMILES: *c1ccc(OC(C)C)cc1C1CC1] | 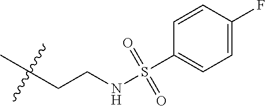 [SMILES: *CCNS(=O)(=O)c1ccc(F)cc1] |
| 20 | 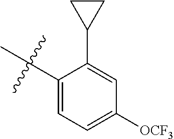 [SMILES: *c1ccc(OC(F)(F)F)cc1C1CC1] | 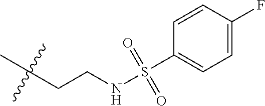 [SMILES: *CCNS(=O)(=O)c1ccc(F)cc1] |
| 21 | 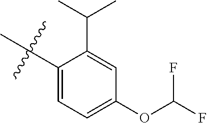 [SMILES: *c1ccc(OC(F)F)cc1C(C)C] | 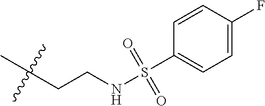 [SMILES: *CCNS(=O)(=O)c1ccc(F)cc1] |
| 22 | 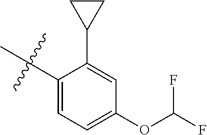 [SMILES: *c1ccc(OC(F)F)cc1C1CC1] | 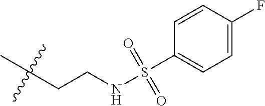 [SMILES: *CCNS(=O)(=O)c1ccc(F)cc1] |
| 23 | 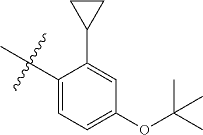 [SMILES: *c1ccc(OC(C)(C)C)cc1C1CC1] | 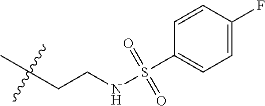 [SMILES: *CCNS(=O)(=O)c1ccc(F)cc1] |
| 24 | 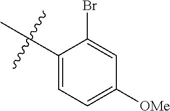 [SMILES: *c1ccc(OC)cc1Br] | 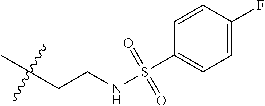 [SMILES: *CCNS(=O)(=O)c1ccc(F)cc1] |
| 25 | 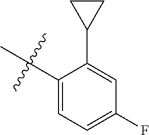 [SMILES: *c1ccc(F)cc1C1CC1] | 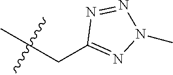 [SMILES: *Cc1nnn(C)n1] |
| 26 | 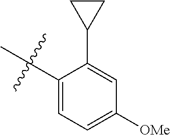 [SMILES: *c1ccc(OC)cc1C1CC1] | 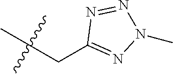 [SMILES: *Cc1nnn(C)n1] |
| 27 | 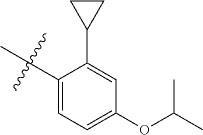 [SMILES: *c1ccc(OC(C)C)cc1C1CC1] | 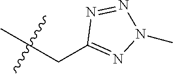 [SMILES: *Cc1nnn(C)n1] |
| 28 | 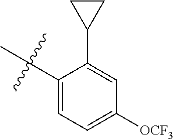 [SMILES: *c1ccc(OC(F)(F)F)cc1C1CC1] | 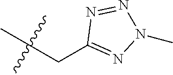 [SMILES: *Cc1nnn(C)n1] |
| 29 | 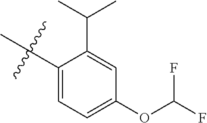 [SMILES: *c1ccc(OC(F)F)cc1C(C)C] | 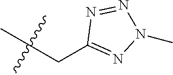 [SMILES: *Cc1nnn(C)n1] |
| 30 | 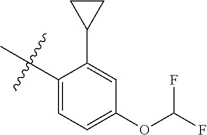 [SMILES: *c1ccc(OC(F)F)cc1C1CC1] | 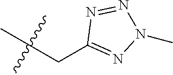 [SMILES: *Cc1nnn(C)n1] |
| 31 | 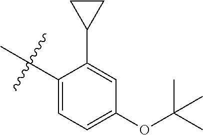 [SMILES: *c1ccc(OC(C)(C)C)cc1C1CC1] | 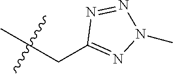 [SMILES: *Cc1nnn(C)n1] |
| 32 | 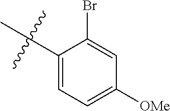 [SMILES: *c1ccc(OC)cc1Br] | 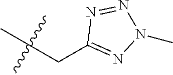 [SMILES: *Cc1nnn(C)n1] |
| 33 | 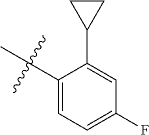 [SMILES: *c1ccc(F)cc1C1CC1] | 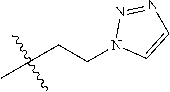 [SMILES: *CCn1ccnn1] |
| 34 | 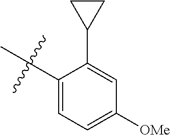 [SMILES: *c1ccc(OC)cc1C1CC1] | 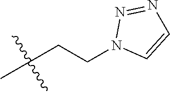 [SMILES: *CCn1ccnn1] |
| 35 | 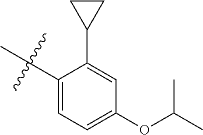 [SMILES: *c1ccc(OC(C)C)cc1C1CC1] | 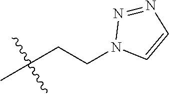 [SMILES: *CCn1ccnn1] |
| 36 | 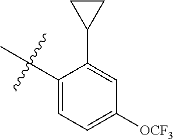 [SMILES: *c1ccc(OC(F)(F)F)cc1C1CC1] | 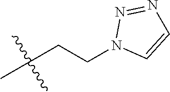 [SMILES: *CCn1ccnn1] |
| 37 | 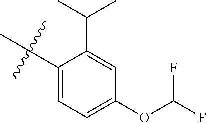 [SMILES: *c1ccc(OC(F)F)cc1C(C)C] | 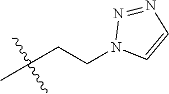 [SMILES: *CCn1ccnn1] |
| 38 | 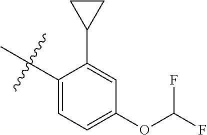 [SMILES: *c1ccc(OC(F)F)cc1C1CC1] | 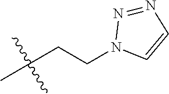 [SMILES: *CCn1ccnn1] |
| 39 | 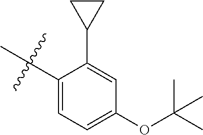 [SMILES: *c1ccc(OC(C)(C)C)cc1C1CC1] | 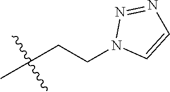 [SMILES: *CCn1ccnn1] |
| 40 | 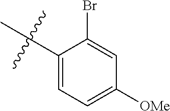 [SMILES: *c1ccc(OC)cc1Br] | 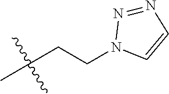 [SMILES: *CCn1ccnn1] |
| 41 | 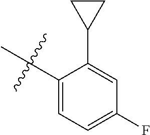 [SMILES: *c1ccc(F)cc1C1CC1] | 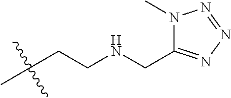 [SMILES: *CCNCc1nnnn1C] |
| 42 | 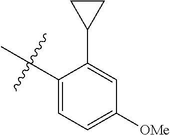 [SMILES: *c1ccc(OC)cc1C1CC1] | 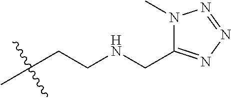 [SMILES: *CCNCc1nnnn1C] |
| 43 | 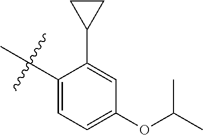 [SMILES: *c1ccc(OC(C)C)cc1C1CC1] | 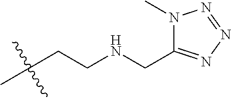 [SMILES: *CCNCc1nnnn1C] |
| 44 | 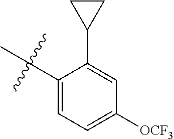 [SMILES: *c1ccc(OC(F)(F)F)cc1C1CC1] | 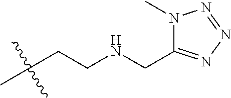 [SMILES: *CCNCc1nnnn1C] |
| 45 | 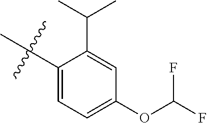 [SMILES: *c1ccc(OC(F)F)cc1C(C)C] | 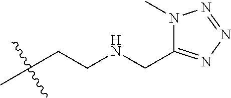 [SMILES: *CCNCc1nnnn1C] |
| 46 | 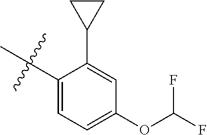 [SMILES: *c1ccc(OC(F)F)cc1C1CC1] | 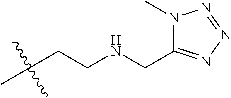 [SMILES: *CCNCc1nnnn1C] |
| 47 | 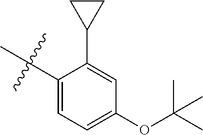 [SMILES: *c1ccc(OC(C)(C)C)cc1C1CC1] | 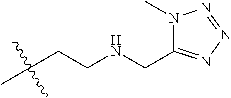 [SMILES: *CCNCc1nnnn1C] |
| 48 | 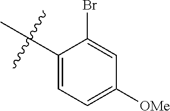 [SMILES: *c1ccc(OC)cc1Br] | 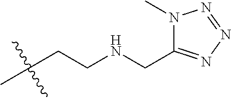 [SMILES: *CCNCc1nnnn1C] |
| 49 | 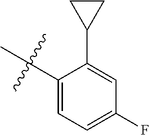 [SMILES: *c1ccc(F)cc1C1CC1] | 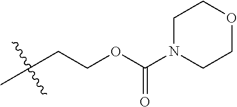 [SMILES: *CCOC(=O)N1CCOCC1] |
| 50 | 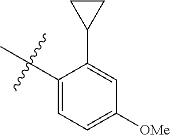 [SMILES: *c1ccc(OC)cc1C1CC1] | 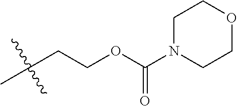 [SMILES: *CCOC(=O)N1CCOCC1] |
| 51 | 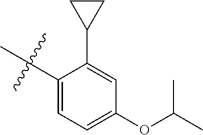 [SMILES: *c1ccc(OC(C)C)cc1C1CC1] | 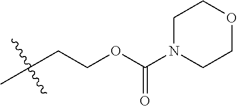 [SMILES: *CCOC(=O)N1CCOCC1] |
| 52 | 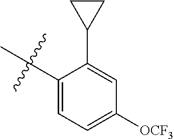 [SMILES: *c1ccc(OC(F)(F)F)cc1C1CC1] | 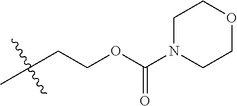 [SMILES: *CCOC(=O)N1CCOCC1] |
| 53 | 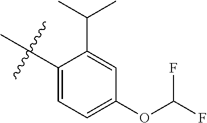 [SMILES: *c1ccc(OC(F)F)cc1C(C)C] | 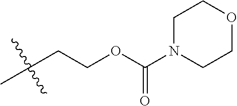 [SMILES: *CCOC(=O)N1CCOCC1] |
| 54 | 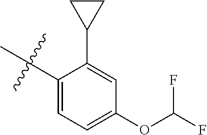 [SMILES: *c1ccc(OC(F)F)cc1C1CC1] | 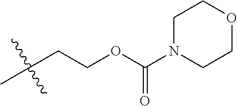 [SMILES: *CCOC(=O)N1CCOCC1] |
| 55 | 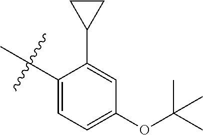 [SMILES: *c1ccc(OC(C)(C)C)cc1C1CC1] | 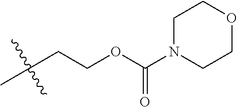 [SMILES: *CCOC(=O)N1CCOCC1] |
| 56 | 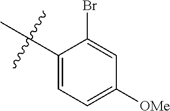 [SMILES: *c1ccc(OC)cc1Br] | 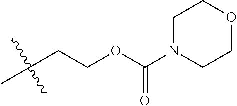 [SMILES: *CCOC(=O)N1CCOCC1] |
| 57 | 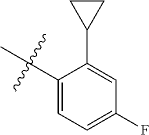 [SMILES: *c1ccc(F)cc1C1CC1] | 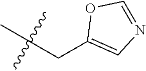 [SMILES: *Cc1cnco1] |
| 58 | 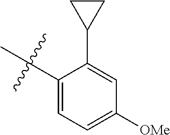 [SMILES: *c1ccc(OC)cc1C1CC1] | 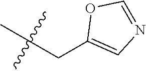 [SMILES: *Cc1cnco1] |
| 59 | 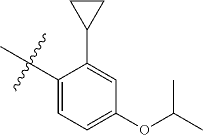 [SMILES: *c1ccc(OC(C)C)cc1C1CC1] | 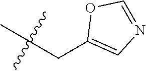 [SMILES: *Cc1cnco1] |
| 60 | 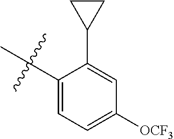 [SMILES: *c1ccc(OC(F)(F)F)cc1C1CC1] | 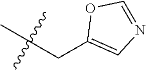 [SMILES: *Cc1cnco1] |
| 61 | 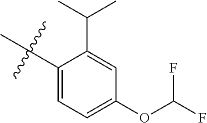 [SMILES: *c1ccc(OC(F)F)cc1C(C)C] | 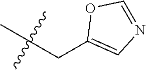 [SMILES: *Cc1cnco1] |
| 62 | 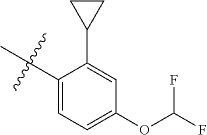 [SMILES: *c1ccc(OC(F)F)cc1C1CC1] | 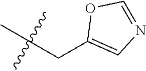 [SMILES: *Cc1cnco1] |
| 63 | 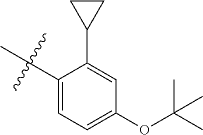 [SMILES: *c1ccc(OC(C)(C)C)cc1C1CC1] | 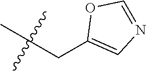 [SMILES: *Cc1cnco1] |
| 64 | 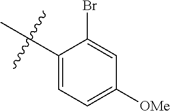 [SMILES: *c1ccc(OC)cc1Br] | 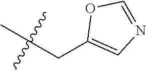 [SMILES: *Cc1cnco1] |
| 65 | 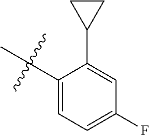 [SMILES: *c1ccc(F)cc1C1CC1] | 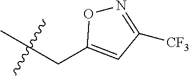 [SMILES: *Cc1cc(C(F)(F)F)no1] |
| 66 | 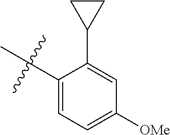 [SMILES: *c1ccc(OC)cc1C1CC1] | 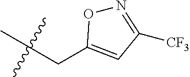 [SMILES: *Cc1cc(C(F)(F)F)no1] |
| 67 | 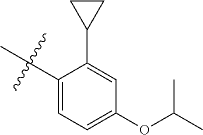 [SMILES: *c1ccc(OC(C)C)cc1C1CC1] | 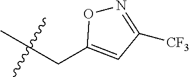 [SMILES: *Cc1cc(C(F)(F)F)no1] |
| 68 | 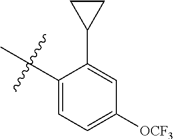 [SMILES: *c1ccc(OC(F)(F)F)cc1C1CC1] | 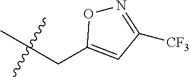 [SMILES: *Cc1cc(C(F)(F)F)no1] |
| 69 | 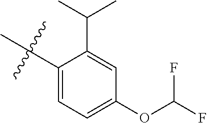 [SMILES: *c1ccc(OC(F)F)cc1C(C)C] | 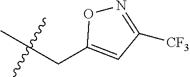 [SMILES: *Cc1cc(C(F)(F)F)no1] |
| 70 | 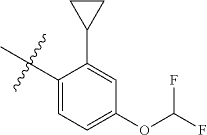 [SMILES: *c1ccc(OC(F)F)cc1C1CC1] | 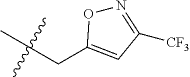 [SMILES: *Cc1cc(C(F)(F)F)no1] |
| 71 | 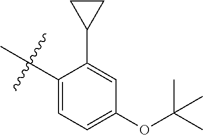 [SMILES: *c1ccc(OC(C)(C)C)cc1C1CC1] | 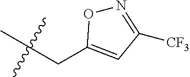 [SMILES: *Cc1cc(C(F)(F)F)no1] |
| 72 | 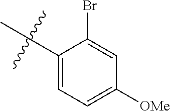 [SMILES: *c1ccc(OC)cc1Br] | 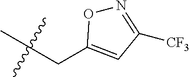 [SMILES: *Cc1cc(C(F)(F)F)no1] |
| 73 | 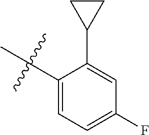 [SMILES: *c1ccc(F)cc1C1CC1] | 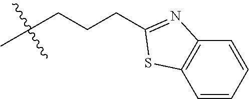 [SMILES: *CCCc1nc2ccccc2s1] |
| 74 | 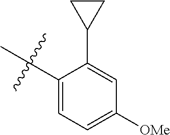 [SMILES: *c1ccc(OC)cc1C1CC1] | 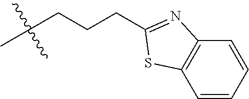 [SMILES: *CCCc1nc2ccccc2s1] |
| 75 | 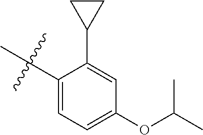 [SMILES: *c1ccc(OC(C)C)cc1C1CC1] | 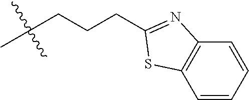 [SMILES: *CCCc1nc2ccccc2s1] |
| 76 | 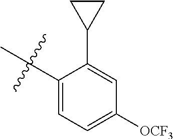 [SMILES: *c1ccc(OC(F)(F)F)cc1C1CC1] | 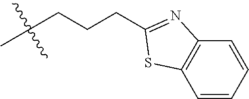 [SMILES: *CCCc1nc2ccccc2s1] |
| 77 | 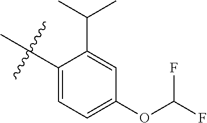 [SMILES: *c1ccc(OC(F)F)cc1C(C)C] | 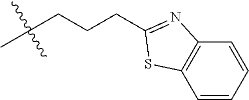 [SMILES: *CCCc1nc2ccccc2s1] |
| 78 | 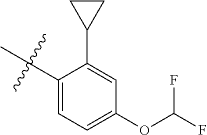 [SMILES: *c1ccc(OC(F)F)cc1C1CC1] | 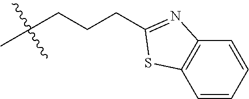 [SMILES: *CCCc1nc2ccccc2s1] |
| 79 | 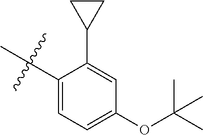 [SMILES: *c1ccc(OC(C)(C)C)cc1C1CC1] | 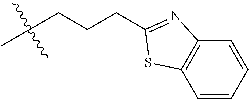 [SMILES: *CCCc1nc2ccccc2s1] |
| 80 | 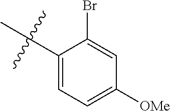 [SMILES: *c1ccc(OC)cc1Br] | 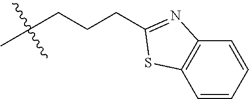 [SMILES: *CCCc1nc2ccccc2s1] |

Representative compounds of the invention include, but are not limited to, the following compounds (Entry 81 to Entry 230 in Table 3) according to Formula (XVI),

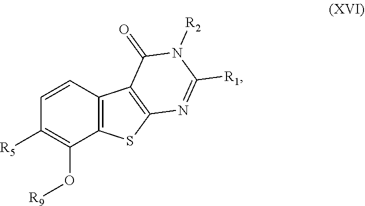

wherein R₅ is not hydrogen, R₁, R₂ and R₉ are delineated for each compound in Table 3. In certain embodiments, R₅ is halogen, such as fluorine or chlorine, methyl, fluoromethyl, difluoromethyl, trifluoromethyl or cyclopropyl, preferably chlorine or fluorine, and more preferably chlorine.

TABLE 3

| Entry | R₁ | R₂ | R₉ |
|---|---|---|---|
| 81 | 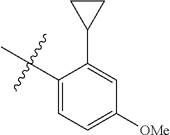 [SMILES: *c1ccc(OC)cc1C1CC1] | 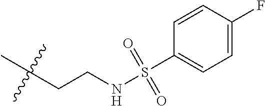 [SMILES: *CCNS(=O)(=O)c1ccc(F)cc1] | 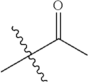 [SMILES: *C(C)=O] |
| 82 | 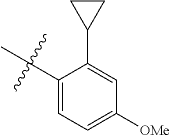 [SMILES: *c1ccc(OC)cc1C1CC1] | 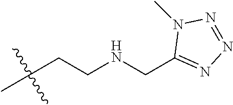 [SMILES: *CCNCc1nnnn1C] | 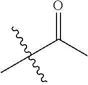 [SMILES: *C(C)=O] |
| 83 | 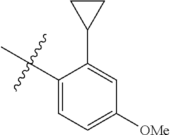 [SMILES: *c1ccc(OC)cc1C1CC1] | 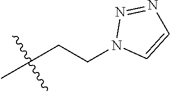 [SMILES: *CCn1ccnn1] | 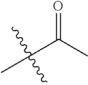 [SMILES: *C(C)=O] |
| 84 | 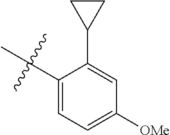 [SMILES: *c1ccc(OC)cc1C1CC1] | 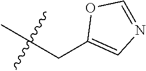 [SMILES: *Cc1cnco1] | 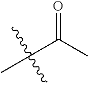 [SMILES: *C(C)=O] |
| 85 | 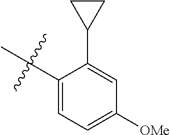 [SMILES: *c1ccc(OC)cc1C1CC1] | 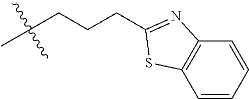 [SMILES: *CCCc1nc2ccccc2s1] | 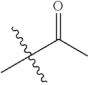 [SMILES: *C(C)=O] |
| 86 | 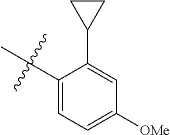 [SMILES: *c1ccc(OC)cc1C1CC1] | 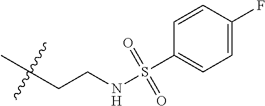 [SMILES: *CCNS(=O)(=O)c1ccc(F)cc1] | 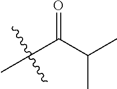 [SMILES: *C(=O)C(C)C] |
| 87 | 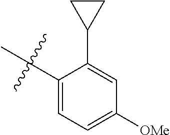 [SMILES: *c1ccc(OC)cc1C1CC1] | 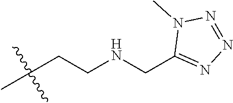 [SMILES: *CCNCc1nnnn1C] | 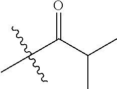 [SMILES: *C(=O)C(C)C] |
| 88 | 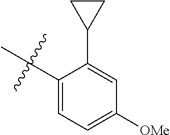 [SMILES: *c1ccc(OC)cc1C1CC1] | 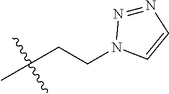 [SMILES: *CCn1ccnn1] | 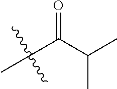 [SMILES: *C(=O)C(C)C] |
| 89 | 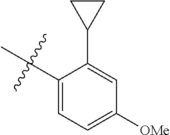 [SMILES: *c1ccc(OC)cc1C1CC1] | 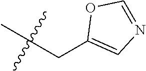 [SMILES: *Cc1cnco1] | 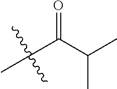 [SMILES: *C(=O)C(C)C] |
| 90 | 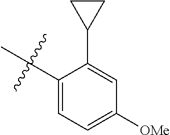 [SMILES: *c1ccc(OC)cc1C1CC1] | 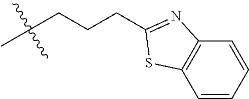 [SMILES: *CCCc1nc2ccccc2s1] | 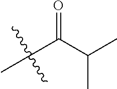 [SMILES: *C(=O)C(C)C] |
| 91 | 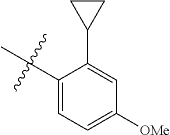 [SMILES: *c1ccc(OC)cc1C1CC1] | 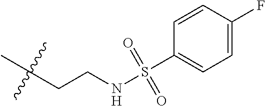 [SMILES: *CCNS(=O)(=O)c1ccc(F)cc1] | 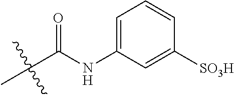 [SMILES: *C(=O)Nc1cccc(S(=O)(=O)O)c1] |
| 92 | 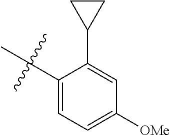 [SMILES: *c1ccc(OC)cc1C1CC1] | 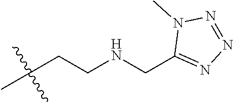 [SMILES: *CCNCc1nnnn1C] | 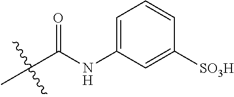 [SMILES: *C(=O)Nc1cccc(S(=O)(=O)O)c1] |
| 93 | 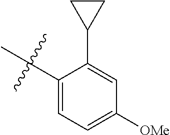 [SMILES: *c1ccc(OC)cc1C1CC1] | 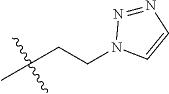 [SMILES: *CCn1ccnn1] | 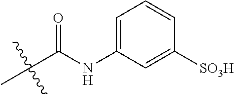 [SMILES: *C(=O)Nc1cccc(S(=O)(=O)O)c1] |
| 94 | 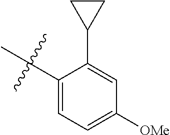 [SMILES: *c1ccc(OC)cc1C1CC1] | 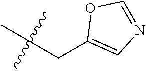 [SMILES: *Cc1cnco1] | 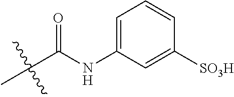 [SMILES: *C(=O)Nc1cccc(S(=O)(=O)O)c1] |
| 95 | 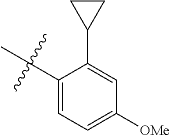 [SMILES: *c1ccc(OC)cc1C1CC1] | 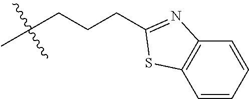 [SMILES: *CCCc1nc2ccccc2s1] | 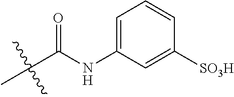 [SMILES: *C(=O)Nc1cccc(S(=O)(=O)O)c1] |
| 96 | 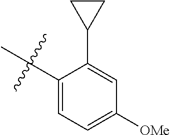 [SMILES: *c1ccc(OC)cc1C1CC1] | 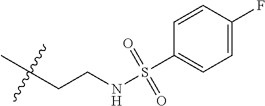 [SMILES: *CCNS(=O)(=O)c1ccc(F)cc1] | 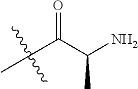 [SMILES: *C(=O)[C@H](C)N] |
| 97 | 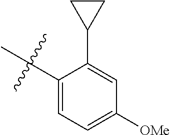 [SMILES: *c1ccc(OC)cc1C1CC1] | 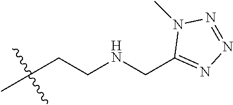 [SMILES: *CCNCc1nnnn1C] | 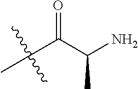 [SMILES: *C(=O)[C@H](C)N] |
| 98 | 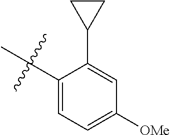 [SMILES: *c1ccc(OC)cc1C1CC1] | 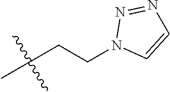 [SMILES: *CCn1ccnn1] | 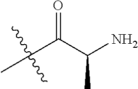 [SMILES: *C(=O)[C@H](C)N] |
| 99 | 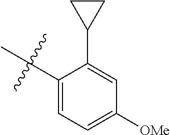 [SMILES: *c1ccc(OC)cc1C1CC1] | 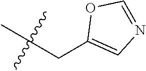 [SMILES: *Cc1cnco1] | 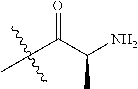 [SMILES: *C(=O)[C@H](C)N] |
| 100 | 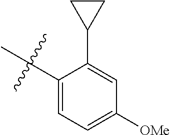 [SMILES: *c1ccc(OC)cc1C1CC1] | 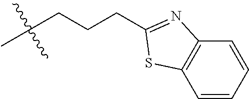 [SMILES: *CCCc1nc2ccccc2s1] | 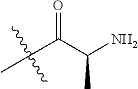 [SMILES: *C(=O)[C@H](C)N] |
| 101 | 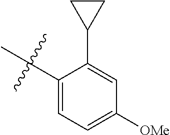 [SMILES: *c1ccc(OC)cc1C1CC1] | 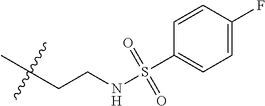 [SMILES: *CCNS(=O)(=O)c1ccc(F)cc1] | 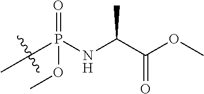 [SMILES: *P(=O)(N[C@@H](C)C(=O)OC)OC] |
| 102 | 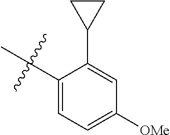 [SMILES: *c1ccc(OC)cc1C1CC1] | 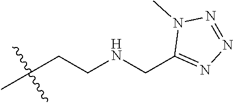 [SMILES: *CCNCc1nnnn1C] | 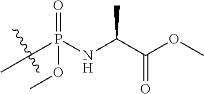 [SMILES: *P(=O)(N[C@@H](C)C(=O)OC)OC] |
| 103 | 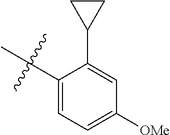 [SMILES: *c1ccc(OC)cc1C1CC1] | 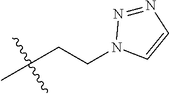 [SMILES: *CCn1ccnn1] | 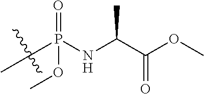 [SMILES: *P(=O)(N[C@@H](C)C(=O)OC)OC] |
| 104 | 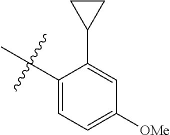 [SMILES: *c1ccc(OC)cc1C1CC1] | 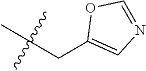 [SMILES: *Cc1cnco1] | 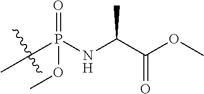 [SMILES: *P(=O)(N[C@@H](C)C(=O)OC)OC] |
| 105 | 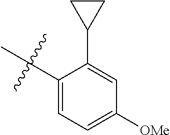 [SMILES: *c1ccc(OC)cc1C1CC1] | 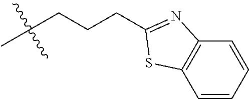 [SMILES: *CCCc1nc2ccccc2s1] | 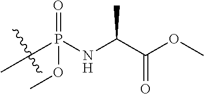 [SMILES: *P(=O)(N[C@@H](C)C(=O)OC)OC] |
| 106 | 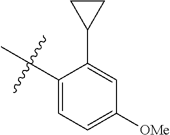 [SMILES: *c1ccc(OC)cc1C1CC1] | 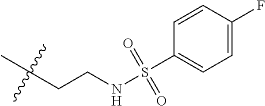 [SMILES: *CCNS(=O)(=O)c1ccc(F)cc1] | 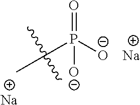 [SMILES: *P(=O)([O-])[O-] ; [Na+] ; [Na+]] |
| 107 | 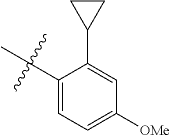 [SMILES: *c1ccc(OC)cc1C1CC1] | 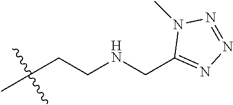 [SMILES: *CCNCc1nnnn1C] | 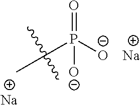 [SMILES: *P(=O)([O-])[O-] ; [Na+] ; [Na+]] |
| 108 | 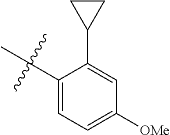 [SMILES: *c1ccc(OC)cc1C1CC1] | 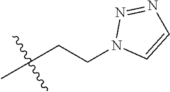 [SMILES: *CCn1ccnn1] | 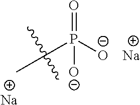 [SMILES: *P(=O)([O-])[O-] ; [Na+] ; [Na+]] |
| 109 | 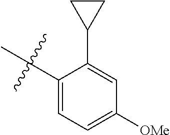 [SMILES: *c1ccc(OC)cc1C1CC1] | 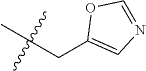 [SMILES: *Cc1cnco1] | 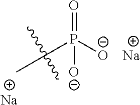 [SMILES: *P(=O)([O-])[O-] ; [Na+] ; [Na+]] |
| 110 | 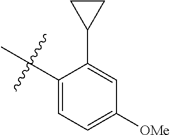 [SMILES: *c1ccc(OC)cc1C1CC1] | 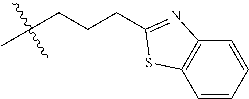 [SMILES: *CCCc1nc2ccccc2s1] | 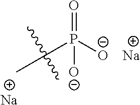 [SMILES: *P(=O)([O-])[O-] ; [Na+] ; [Na+]] |
| 111 | 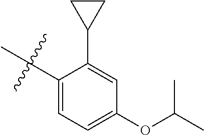 [SMILES: *c1ccc(OC(C)C)cc1C1CC1] | 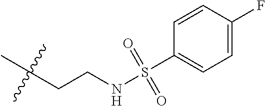 [SMILES: *CCNS(=O)(=O)c1ccc(F)cc1] | 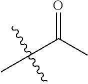 [SMILES: *C(C)=O] |
| 112 | 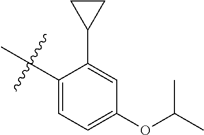 [SMILES: *c1ccc(OC(C)C)cc1C1CC1] | 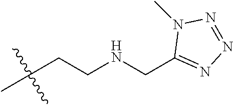 [SMILES: *CCNCc1nnnn1C] | 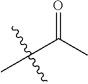 [SMILES: *C(C)=O] |
| 113 | 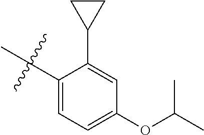 [SMILES: *c1ccc(OC(C)C)cc1C1CC1] | 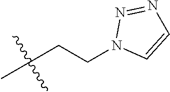 [SMILES: *CCn1ccnn1] | 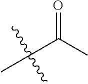 [SMILES: *C(C)=O] |
| 114 | 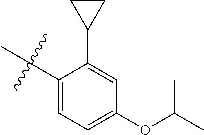 [SMILES: *c1ccc(OC(C)C)cc1C1CC1] | 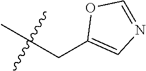 [SMILES: *Cc1cnco1] | 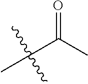 [SMILES: *C(C)=O] |
| 115 | 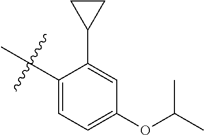 [SMILES: *c1ccc(OC(C)C)cc1C1CC1] | 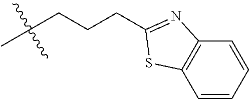 [SMILES: *CCCc1nc2ccccc2s1] | 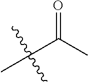 [SMILES: *C(C)=O] |
| 116 | 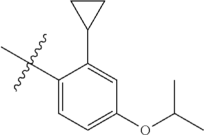 [SMILES: *c1ccc(OC(C)C)cc1C1CC1] | 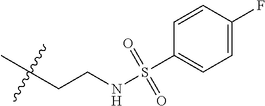 [SMILES: *CCNS(=O)(=O)c1ccc(F)cc1] | 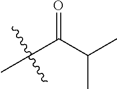 [SMILES: *C(=O)C(C)C] |
| 117 | 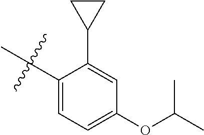 [SMILES: *c1ccc(OC(C)C)cc1C1CC1] | 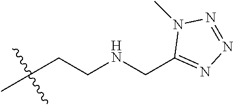 [SMILES: *CCNCc1nnnn1C] | 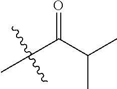 [SMILES: *C(=O)C(C)C] |
| 118 | 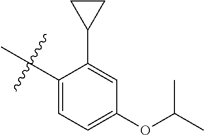 [SMILES: *c1ccc(OC(C)C)cc1C1CC1] | 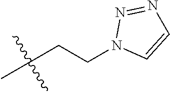 [SMILES: *CCn1ccnn1] | 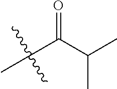 [SMILES: *C(=O)C(C)C] |
| 119 | 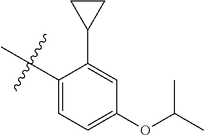 [SMILES: *c1ccc(OC(C)C)cc1C1CC1] | 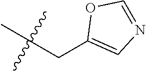 [SMILES: *Cc1cnco1] | 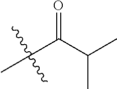 [SMILES: *C(=O)C(C)C] |
| 120 | 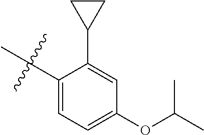 [SMILES: *c1ccc(OC(C)C)cc1C1CC1] | 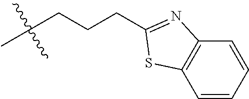 [SMILES: *CCCc1nc2ccccc2s1] | 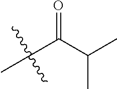 [SMILES: *C(=O)C(C)C] |
| 121 | 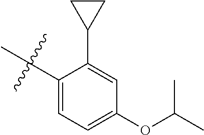 [SMILES: *c1ccc(OC(C)C)cc1C1CC1] | 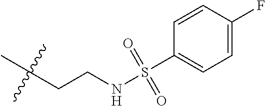 [SMILES: *CCNS(=O)(=O)c1ccc(F)cc1] | 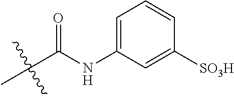 [SMILES: *C(=O)Nc1cccc(S(=O)(=O)O)c1] |
| 122 | 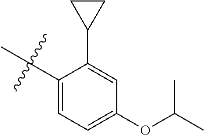 [SMILES: *c1ccc(OC(C)C)cc1C1CC1] | 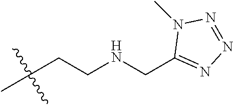 [SMILES: *CCNCc1nnnn1C] | 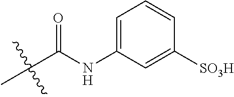 [SMILES: *C(=O)Nc1cccc(S(=O)(=O)O)c1] |
| 123 | 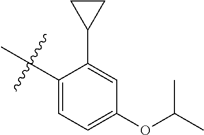 [SMILES: *c1ccc(OC(C)C)cc1C1CC1] | 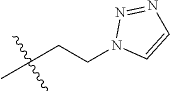 [SMILES: *CCn1ccnn1] | 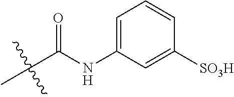 [SMILES: *C(=O)Nc1cccc(S(=O)(=O)O)c1] |
| 124 | 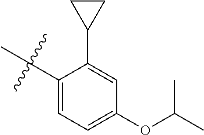 [SMILES: *c1ccc(OC(C)C)cc1C1CC1] | 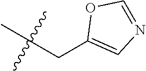 [SMILES: *Cc1cnco1] | 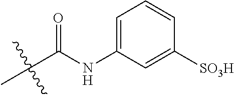 [SMILES: *C(=O)Nc1cccc(S(=O)(=O)O)c1] |
| 125 | 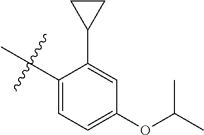 [SMILES: *c1ccc(OC(C)C)cc1C1CC1] | 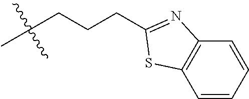 [SMILES: *CCCc1nc2ccccc2s1] | 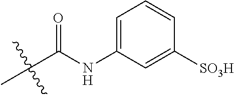 [SMILES: *C(=O)Nc1cccc(S(=O)(=O)O)c1] |
| 126 | 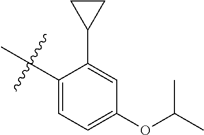 [SMILES: *c1ccc(OC(C)C)cc1C1CC1] | 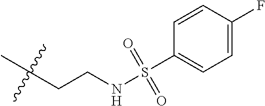 [SMILES: *CCNS(=O)(=O)c1ccc(F)cc1] | 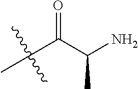 [SMILES: *C(=O)[C@H](C)N] |
| 127 | 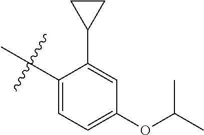 [SMILES: *c1ccc(OC(C)C)cc1C1CC1] | 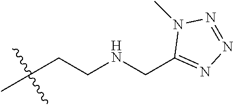 [SMILES: *CCNCc1nnnn1C] | 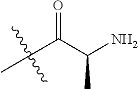 [SMILES: *C(=O)[C@H](C)N] |
| 128 | 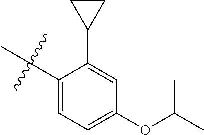 [SMILES: *c1ccc(OC(C)C)cc1C1CC1] | 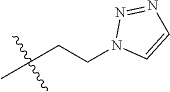 [SMILES: *CCn1ccnn1] | 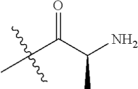 [SMILES: *C(=O)[C@H](C)N] |
| 129 | 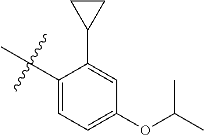 [SMILES: *c1ccc(OC(C)C)cc1C1CC1] | 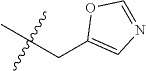 [SMILES: *Cc1cnco1] | 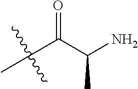 [SMILES: *C(=O)[C@H](C)N] |
| 130 | 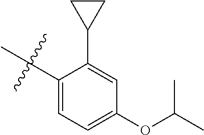 [SMILES: *c1ccc(OC(C)C)cc1C1CC1] | 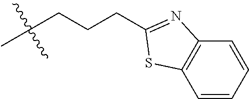 [SMILES: *CCCc1nc2ccccc2s1] | 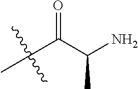 [SMILES: *C(=O)[C@H](C)N] |
| 131 | 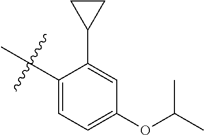 [SMILES: *c1ccc(OC(C)C)cc1C1CC1] | 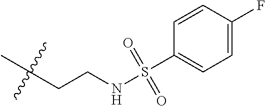 [SMILES: *CCNS(=O)(=O)c1ccc(F)cc1] | 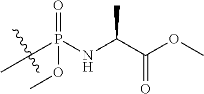 [SMILES: *P(=O)(N[C@@H](C)C(=O)OC)OC] |
| 132 | 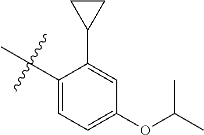 [SMILES: *c1ccc(OC(C)C)cc1C1CC1] | 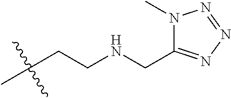 [SMILES: *CCNCc1nnnn1C] | 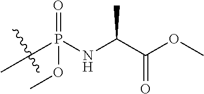 [SMILES: *P(=O)(N[C@@H](C)C(=O)OC)OC] |
| 133 | 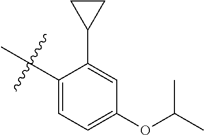 [SMILES: *c1ccc(OC(C)C)cc1C1CC1] | 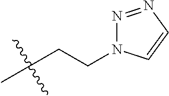 [SMILES: *CCn1ccnn1] | 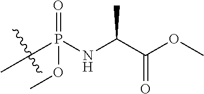 [SMILES: *P(=O)(N[C@@H](C)C(=O)OC)OC] |
| 134 | 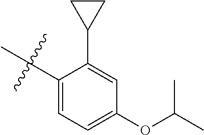 [SMILES: *c1ccc(OC(C)C)cc1C1CC1] | 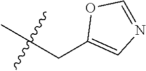 [SMILES: *Cc1cnco1] | 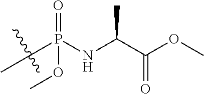 [SMILES: *P(=O)(N[C@@H](C)C(=O)OC)OC] |
| 135 | 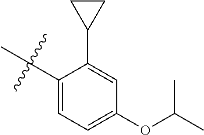 [SMILES: *c1ccc(OC(C)C)cc1C1CC1] | 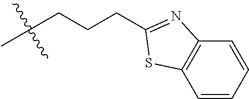 [SMILES: *CCCc1nc2ccccc2s1] | 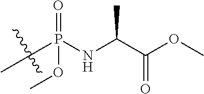 [SMILES: *P(=O)(N[C@@H](C)C(=O)OC)OC] |
| 136 | 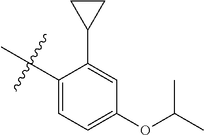 [SMILES: *c1ccc(OC(C)C)cc1C1CC1] | 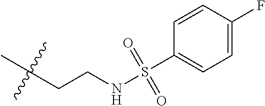 [SMILES: *CCNS(=O)(=O)c1ccc(F)cc1] | 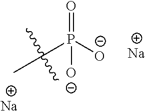 [SMILES: *P(=O)([O-])[O-] ; [Na+] ; [Na+]] |
| 137 | 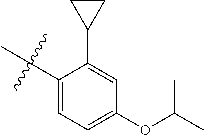 [SMILES: *c1ccc(OC(C)C)cc1C1CC1] | 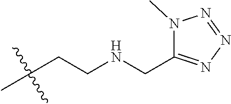 [SMILES: *CCNCc1nnnn1C] | 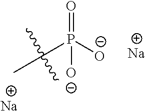 [SMILES: *P(=O)([O-])[O-] ; [Na+] ; [Na+]] |
| 138 | 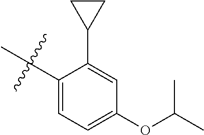 [SMILES: *c1ccc(OC(C)C)cc1C1CC1] | 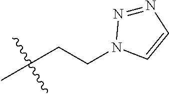 [SMILES: *CCn1ccnn1] | 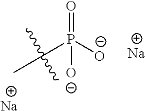 [SMILES: *P(=O)([O-])[O-] ; [Na+] ; [Na+]] |
| 139 | 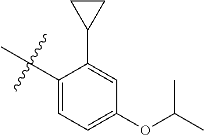 [SMILES: *c1ccc(OC(C)C)cc1C1CC1] | 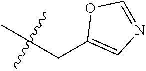 [SMILES: *Cc1cnco1] | 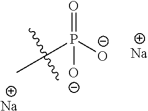 [SMILES: *P(=O)([O-])[O-] ; [Na+] ; [Na+]] |
| 140 | 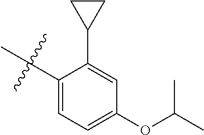 [SMILES: *c1ccc(OC(C)C)cc1C1CC1] | 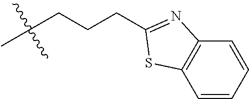 [SMILES: *CCCc1nc2ccccc2s1] | 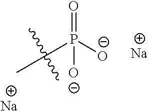 [SMILES: *P(=O)([O-])[O-] ; [Na+] ; [Na+]] |
| 141 | 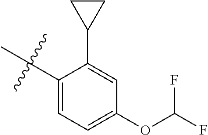 [SMILES: *c1ccc(OC(F)F)cc1C1CC1] | 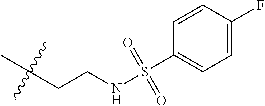 [SMILES: *CCNS(=O)(=O)c1ccc(F)cc1] | 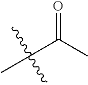 [SMILES: *C(C)=O] |
| 142 | 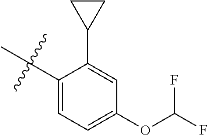 [SMILES: *c1ccc(OC(F)F)cc1C1CC1] | 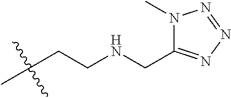 [SMILES: *CCNCc1nnnn1C] | 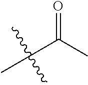 [SMILES: *C(C)=O] |
| 143 | 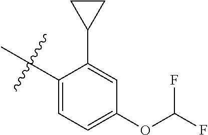 [SMILES: *c1ccc(OC(F)F)cc1C1CC1] | 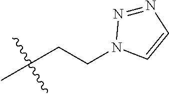 [SMILES: *CCn1ccnn1] | 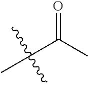 [SMILES: *C(C)=O] |
| 144 | 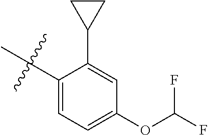 [SMILES: *c1ccc(OC(F)F)cc1C1CC1] | 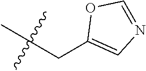 [SMILES: *Cc1cnco1] | 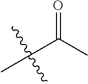 [SMILES: *C(C)=O] |
| 145 | 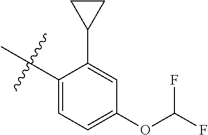 [SMILES: *c1ccc(OC(F)F)cc1C1CC1] | 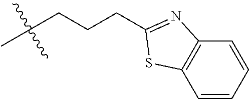 [SMILES: *CCCc1nc2ccccc2s1] | 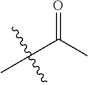 [SMILES: *C(C)=O] |
| 146 | 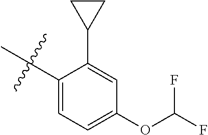 [SMILES: *c1ccc(OC(F)F)cc1C1CC1] | 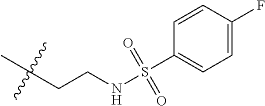 [SMILES: *CCNS(=O)(=O)c1ccc(F)cc1] | 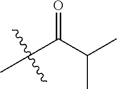 [SMILES: *C(=O)C(C)C] |
| 147 | 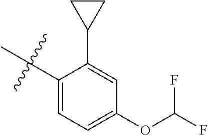 [SMILES: *c1ccc(OC(F)F)cc1C1CC1] | 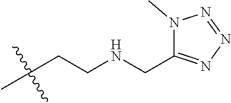 [SMILES: *CCNCc1nnnn1C] | 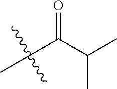 [SMILES: *C(=O)C(C)C] |
| 148 | 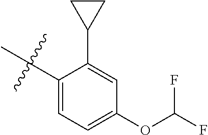 [SMILES: *c1ccc(OC(F)F)cc1C1CC1] | 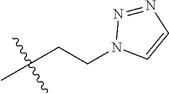 [SMILES: *CCn1ccnn1] | 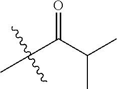 [SMILES: *C(=O)C(C)C] |
| 149 | 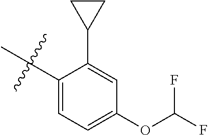 [SMILES: *c1ccc(OC(F)F)cc1C1CC1] | 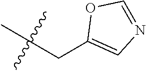 [SMILES: *Cc1cnco1] | 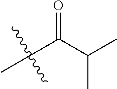 [SMILES: *C(=O)C(C)C] |
| 150 | 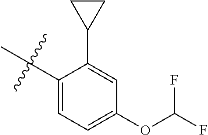 [SMILES: *c1ccc(OC(F)F)cc1C1CC1] | 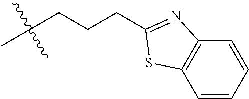 [SMILES: *CCCc1nc2ccccc2s1] | 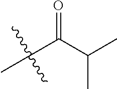 [SMILES: *C(=O)C(C)C] |
| 151 | 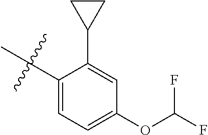 [SMILES: *c1ccc(OC(F)F)cc1C1CC1] | 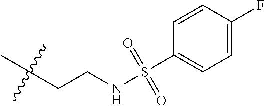 [SMILES: *CCNS(=O)(=O)c1ccc(F)cc1] | 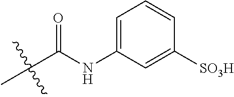 [SMILES: *C(=O)Nc1cccc(S(=O)(=O)O)c1] |
| 152 | 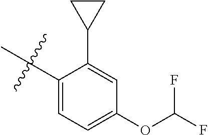 [SMILES: *c1ccc(OC(F)F)cc1C1CC1] | 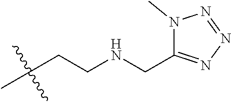 [SMILES: *CCNCc1nnnn1C] | 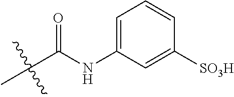 [SMILES: *C(=O)Nc1cccc(S(=O)(=O)O)c1] |
| 153 | 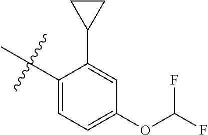 [SMILES: *c1ccc(OC(F)F)cc1C1CC1] | 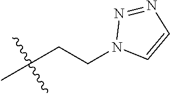 [SMILES: *CCn1ccnn1] | 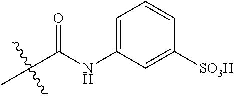 [SMILES: *C(=O)Nc1cccc(S(=O)(=O)O)c1] |
| 154 | 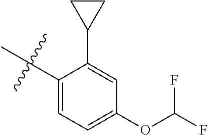 [SMILES: *c1ccc(OC(F)F)cc1C1CC1] | 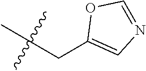 [SMILES: *Cc1cnco1] | 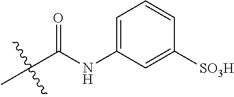 [SMILES: *C(=O)Nc1cccc(S(=O)(=O)O)c1] |
| 155 | 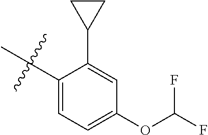 [SMILES: *c1ccc(OC(F)F)cc1C1CC1] | 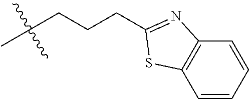 [SMILES: *CCCc1nc2ccccc2s1] | 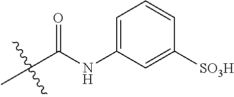 [SMILES: *C(=O)Nc1cccc(S(=O)(=O)O)c1] |
| 156 | 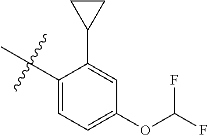 [SMILES: *c1ccc(OC(F)F)cc1C1CC1] | 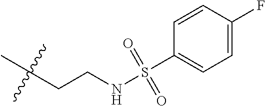 [SMILES: *CCNS(=O)(=O)c1ccc(F)cc1] | 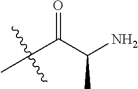 [SMILES: *C(=O)[C@H](C)N] |
| 157 | 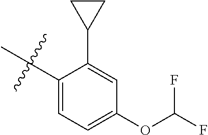 [SMILES: *c1ccc(OC(F)F)cc1C1CC1] | 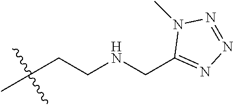 [SMILES: *CCNCc1nnnn1C] | 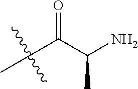 [SMILES: *C(=O)[C@H](C)N] |
| 158 | 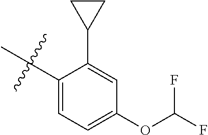 [SMILES: *c1ccc(OC(F)F)cc1C1CC1] | 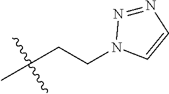 [SMILES: *CCn1ccnn1] | 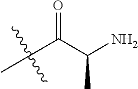 [SMILES: *C(=O)[C@H](C)N] |
| 159 | 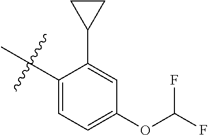 [SMILES: *c1ccc(OC(F)F)cc1C1CC1] | 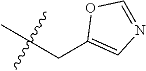 [SMILES: *Cc1cnco1] | 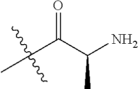 [SMILES: *C(=O)[C@H](C)N] |
| 160 | 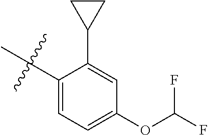 [SMILES: *c1ccc(OC(F)F)cc1C1CC1] | 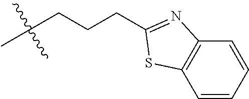 [SMILES: *CCCc1nc2ccccc2s1] | 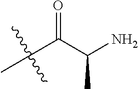 [SMILES: *C(=O)[C@H](C)N] |
| 161 | 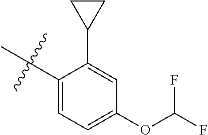 [SMILES: *c1ccc(OC(F)F)cc1C1CC1] | 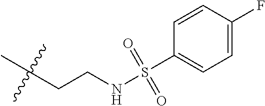 [SMILES: *CCNS(=O)(=O)c1ccc(F)cc1] | 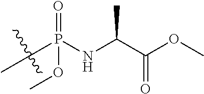 [SMILES: *P(=O)(N[C@@H](C)C(=O)OC)OC] |
| 162 | 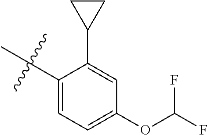 [SMILES: *c1ccc(OC(F)F)cc1C1CC1] | 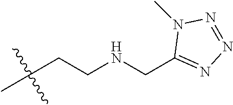 [SMILES: *CCNCc1nnnn1C] | 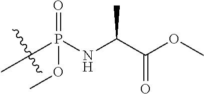 [SMILES: *P(=O)(N[C@@H](C)C(=O)OC)OC] |
| 163 | 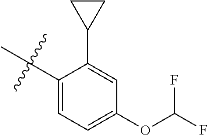 [SMILES: *c1ccc(OC(F)F)cc1C1CC1] | 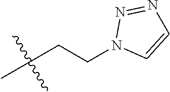 [SMILES: *CCn1ccnn1] | 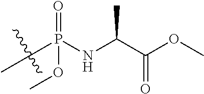 [SMILES: *P(=O)(N[C@@H](C)C(=O)OC)OC] |
| 164 | 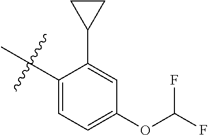 [SMILES: *c1ccc(OC(F)F)cc1C1CC1] | 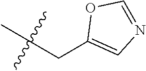 [SMILES: *Cc1cnco1] | 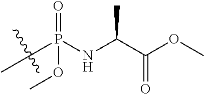 [SMILES: *P(=O)(N[C@@H](C)C(=O)OC)OC] |
| 165 | 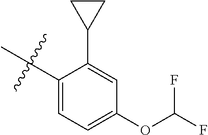 [SMILES: *c1ccc(OC(F)F)cc1C1CC1] | 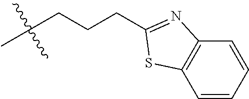 [SMILES: *CCCc1nc2ccccc2s1] | 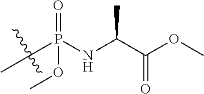 [SMILES: *P(=O)(N[C@@H](C)C(=O)OC)OC] |
| 166 | 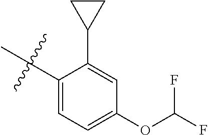 [SMILES: *c1ccc(OC(F)F)cc1C1CC1] | 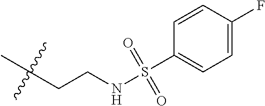 [SMILES: *CCNS(=O)(=O)c1ccc(F)cc1] | 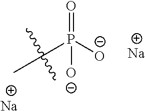 [SMILES: *P(=O)([O-])[O-] ; [Na+] ; [Na+]] |
| 167 | 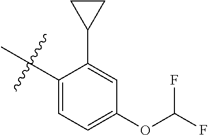 [SMILES: *c1ccc(OC(F)F)cc1C1CC1] | 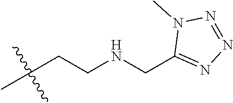 [SMILES: *CCNCc1nnnn1C] | 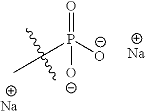 [SMILES: *P(=O)([O-])[O-] ; [Na+] ; [Na+]] |
| 168 | 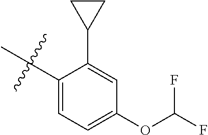 [SMILES: *c1ccc(OC(F)F)cc1C1CC1] | 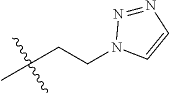 [SMILES: *CCn1ccnn1] | 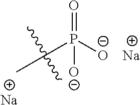 [SMILES: *P(=O)([O-])[O-] ; [Na+] ; [Na+]] |
| 169 | 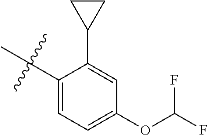 [SMILES: *c1ccc(OC(F)F)cc1C1CC1] | 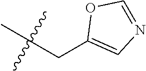 [SMILES: *Cc1cnco1] | 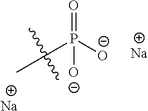 [SMILES: *P(=O)([O-])[O-] ; [Na+] ; [Na+]] |
| 170 | 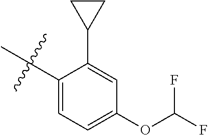 [SMILES: *c1ccc(OC(F)F)cc1C1CC1] | 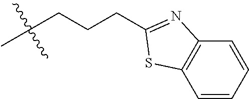 [SMILES: *CCCc1nc2ccccc2s1] | 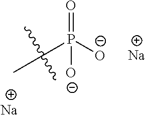 [SMILES: *P(=O)([O-])[O-] ; [Na+] ; [Na+]] |
| 171 | 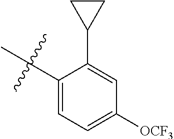 [SMILES: *c1ccc(OC(F)(F)F)cc1C1CC1] | 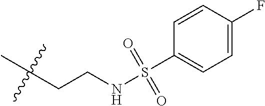 [SMILES: *CCNS(=O)(=O)c1ccc(F)cc1] | 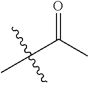 [SMILES: *C(C)=O] |
| 172 | 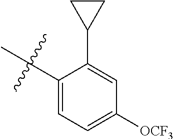 [SMILES: *c1ccc(OC(F)(F)F)cc1C1CC1] | 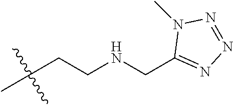 [SMILES: *CCNCc1nnnn1C] | 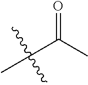 [SMILES: *C(C)=O] |
| 173 | 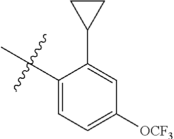 [SMILES: *c1ccc(OC(F)(F)F)cc1C1CC1] | 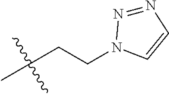 [SMILES: *CCn1ccnn1] | 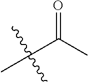 [SMILES: *C(C)=O] |
| 174 | 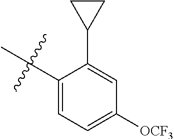 [SMILES: *c1ccc(OC(F)(F)F)cc1C1CC1] | 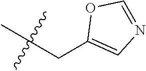 [SMILES: *Cc1cnco1] | 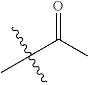 [SMILES: *C(C)=O] |
| 175 | 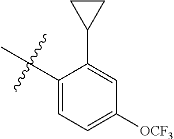 [SMILES: *c1ccc(OC(F)(F)F)cc1C1CC1] | 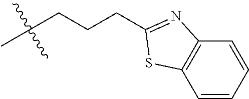 [SMILES: *CCCc1nc2ccccc2s1] | 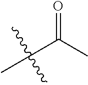 [SMILES: *C(C)=O] |
| 176 | 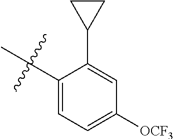 [SMILES: *c1ccc(OC(F)(F)F)cc1C1CC1] | 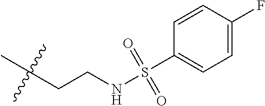 [SMILES: *CCNS(=O)(=O)c1ccc(F)cc1] | 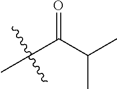 [SMILES: *C(=O)C(C)C] |
| 177 | 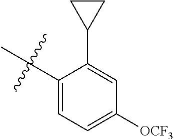 [SMILES: *c1ccc(OC(F)(F)F)cc1C1CC1] | 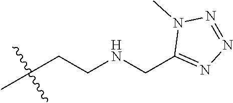 [SMILES: *CCNCc1nnnn1C] | 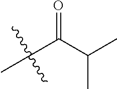 [SMILES: *C(=O)C(C)C] |
| 178 | 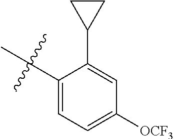 [SMILES: *c1ccc(OC(F)(F)F)cc1C1CC1] | 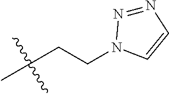 [SMILES: *CCn1ccnn1] | 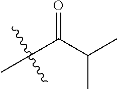 [SMILES: *C(=O)C(C)C] |
| 179 | 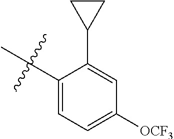 [SMILES: *c1ccc(OC(F)(F)F)cc1C1CC1] | 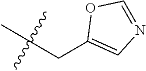 [SMILES: *Cc1cnco1] | 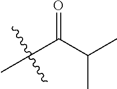 [SMILES: *C(=O)C(C)C] |
| 180 | 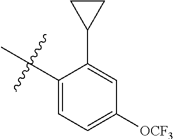 [SMILES: *c1ccc(OC(F)(F)F)cc1C1CC1] | 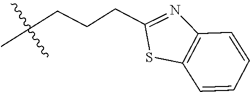 [SMILES: *CCCc1nc2ccccc2s1] | 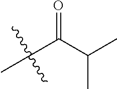 [SMILES: *C(=O)C(C)C] |
| 181 | 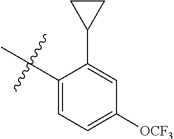 [SMILES: *c1ccc(OC(F)(F)F)cc1C1CC1] | 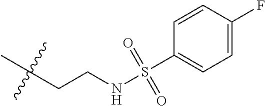 [SMILES: *CCNS(=O)(=O)c1ccc(F)cc1] | 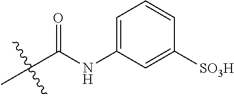 [SMILES: *C(=O)Nc1cccc(S(=O)(=O)O)c1] |
| 182 | 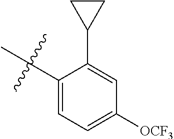 [SMILES: *c1ccc(OC(F)(F)F)cc1C1CC1] | 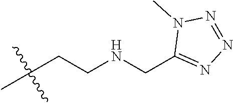 [SMILES: *CCNCc1nnnn1C] | 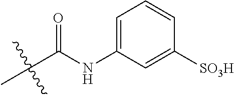 [SMILES: *C(=O)Nc1cccc(S(=O)(=O)O)c1] |
| 183 | 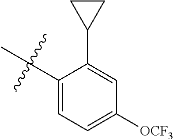 [SMILES: *c1ccc(OC(F)(F)F)cc1C1CC1] | 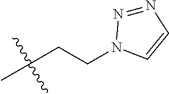 [SMILES: *CCn1ccnn1] | 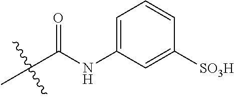 [SMILES: *C(=O)Nc1cccc(S(=O)(=O)O)c1] |
| 184 | 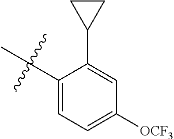 [SMILES: *c1ccc(OC(F)(F)F)cc1C1CC1] | 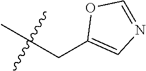 [SMILES: *Cc1cnco1] | 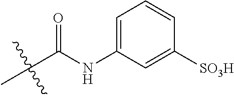 [SMILES: *C(=O)Nc1cccc(S(=O)(=O)O)c1] |
| 185 | 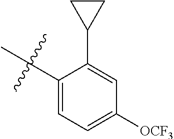 [SMILES: *c1ccc(OC(F)(F)F)cc1C1CC1] | 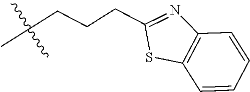 [SMILES: *CCCc1nc2ccccc2s1] | 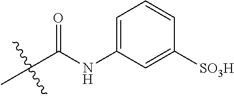 [SMILES: *C(=O)Nc1cccc(S(=O)(=O)O)c1] |
| 186 | 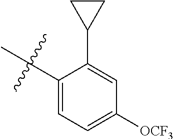 [SMILES: *c1ccc(OC(F)(F)F)cc1C1CC1] | 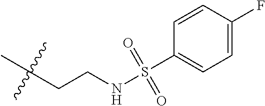 [SMILES: *CCNS(=O)(=O)c1ccc(F)cc1] | 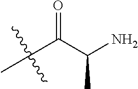 [SMILES: *C(=O)[C@H](C)N] |
| 187 | 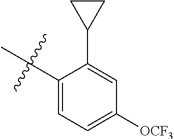 [SMILES: *c1ccc(OC(F)(F)F)cc1C1CC1] | 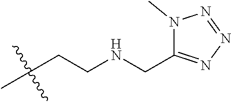 [SMILES: *CCNCc1nnnn1C] | 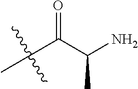 [SMILES: *C(=O)[C@H](C)N] |
| 188 | 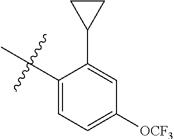 [SMILES: *c1ccc(OC(F)(F)F)cc1C1CC1] | 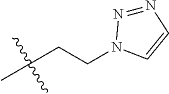 [SMILES: *CCn1ccnn1] | 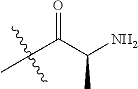 [SMILES: *C(=O)[C@H](C)N] |
| 189 | 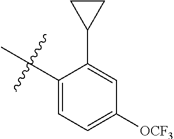 [SMILES: *c1ccc(OC(F)(F)F)cc1C1CC1] | 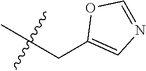 [SMILES: *Cc1cnco1] | 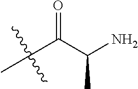 [SMILES: *C(=O)[C@H](C)N] |
| 190 | 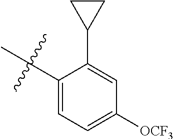 [SMILES: *c1ccc(OC(F)(F)F)cc1C1CC1] | 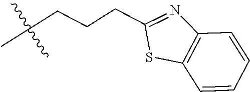 [SMILES: *CCCc1nc2ccccc2s1] | 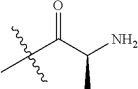 [SMILES: *C(=O)[C@H](C)N] |
| 191 | 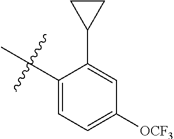 [SMILES: *c1ccc(OC(F)(F)F)cc1C1CC1] | 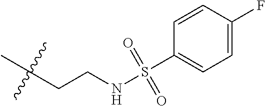 [SMILES: *CCNS(=O)(=O)c1ccc(F)cc1] | 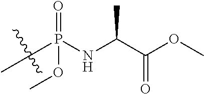 [SMILES: *P(=O)(N[C@@H](C)C(=O)OC)OC] |
| 192 | 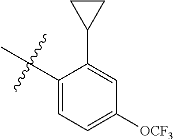 [SMILES: *c1ccc(OC(F)(F)F)cc1C1CC1] | 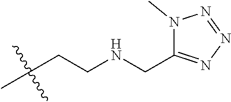 [SMILES: *CCNCc1nnnn1C] | 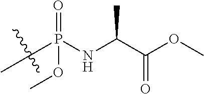 [SMILES: *P(=O)(N[C@@H](C)C(=O)OC)OC] |
| 193 | 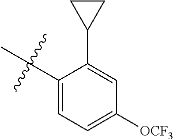 [SMILES: *c1ccc(OC(F)(F)F)cc1C1CC1] | 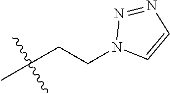 [SMILES: *CCn1ccnn1] | 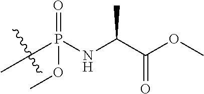 [SMILES: *P(=O)(N[C@@H](C)C(=O)OC)OC] |
| 194 | 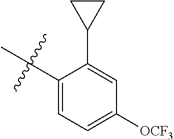 [SMILES: *c1ccc(OC(F)(F)F)cc1C1CC1] | 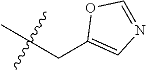 [SMILES: *Cc1cnco1] | 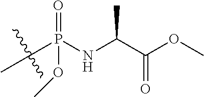 [SMILES: *P(=O)(N[C@@H](C)C(=O)OC)OC] |
| 195 | 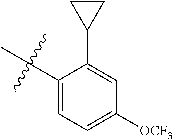 [SMILES: *c1ccc(OC(F)(F)F)cc1C1CC1] | 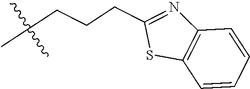 [SMILES: *CCCc1nc2ccccc2s1] | 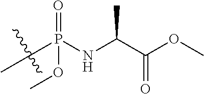 [SMILES: *P(=O)(N[C@@H](C)C(=O)OC)OC] |
| 196 | 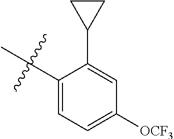 [SMILES: *c1ccc(OC(F)(F)F)cc1C1CC1] | 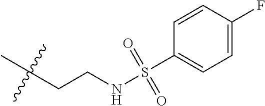 [SMILES: *CCNS(=O)(=O)c1ccc(F)cc1] | 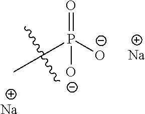 [SMILES: *P(=O)([O-])[O-] ; [Na+] ; [Na+]] |
| 197 | 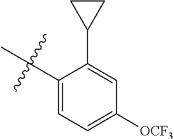 [SMILES: *c1ccc(OC(F)(F)F)cc1C1CC1] | 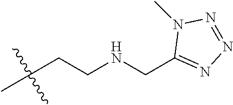 [SMILES: *CCNCc1nnnn1C] | 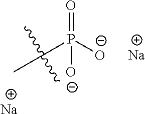 [SMILES: *P(=O)([O-])[O-] ; [Na+] ; [Na+]] |
| 198 | 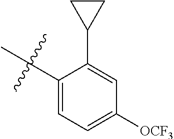 [SMILES: *c1ccc(OC(F)(F)F)cc1C1CC1] | 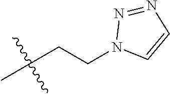 [SMILES: *CCn1ccnn1] | 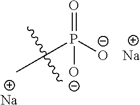 [SMILES: *P(=O)([O-])[O-] ; [Na+] ; [Na+]] |
| 199 | 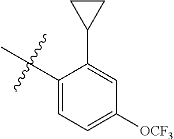 [SMILES: *c1ccc(OC(F)(F)F)cc1C1CC1] | 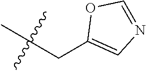 [SMILES: *Cc1cnco1] | 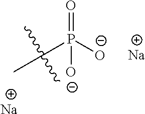 [SMILES: *P(=O)([O-])[O-] ; [Na+] ; [Na+]] |
| 200 | 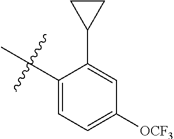 [SMILES: *c1ccc(OC(F)(F)F)cc1C1CC1] | 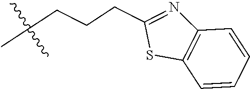 [SMILES: *CCCc1nc2ccccc2s1] | 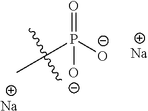 [SMILES: *P(=O)([O-])[O-] ; [Na+] ; [Na+]] |
| 201 | 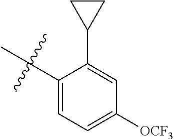 [SMILES: *c1ccc(OC(F)(F)F)cc1C1CC1] | 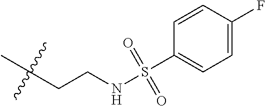 [SMILES: *CCNS(=O)(=O)c1ccc(F)cc1] | 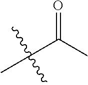 [SMILES: *C(C)=O] |
| 202 | 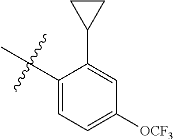 [SMILES: *c1ccc(OC(F)(F)F)cc1C1CC1] | 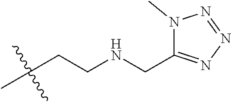 [SMILES: *CCNCc1nnnn1C] | 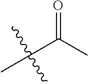 [SMILES: *C(C)=O] |
| 203 | 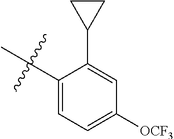 [SMILES: *c1ccc(OC(F)(F)F)cc1C1CC1] | 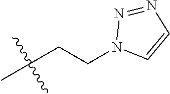 [SMILES: *CCn1ccnn1] | 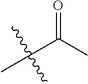 [SMILES: *C(C)=O] |
| 204 | 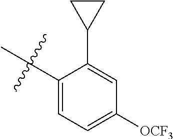 [SMILES: *c1ccc(OC(F)(F)F)cc1C1CC1] | 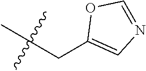 [SMILES: *Cc1cnco1] | 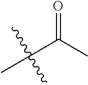 [SMILES: *C(C)=O] |
| 205 | 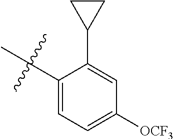 [SMILES: *c1ccc(OC(F)(F)F)cc1C1CC1] | 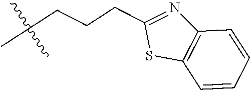 [SMILES: *CCCc1nc2ccccc2s1] | 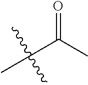 [SMILES: *C(C)=O] |
| 206 | 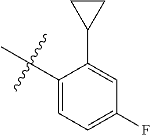 [SMILES: *c1ccc(F)cc1C1CC1] | 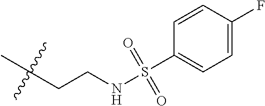 [SMILES: *CCNS(=O)(=O)c1ccc(F)cc1] | 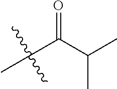 [SMILES: *C(=O)C(C)C] |
| 207 | 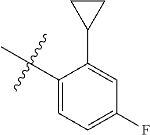 [SMILES: *c1ccc(F)cc1C1CC1] | 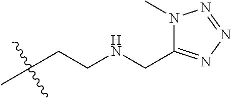 [SMILES: *CCNCc1nnnn1C] | 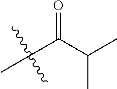 [SMILES: *C(=O)C(C)C] |
| 208 | 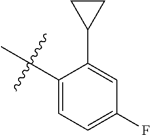 [SMILES: *c1ccc(F)cc1C1CC1] | 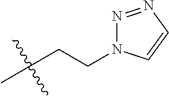 [SMILES: *CCn1ccnn1] | 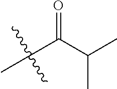 [SMILES: *C(=O)C(C)C] |
| 209 | 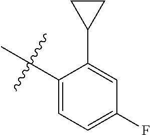 [SMILES: *c1ccc(F)cc1C1CC1] | 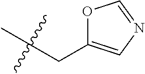 [SMILES: *Cc1cnco1] | 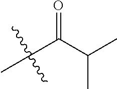 [SMILES: *C(=O)C(C)C] |
| 210 | 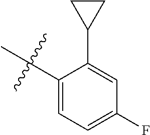 [SMILES: *c1ccc(F)cc1C1CC1] | 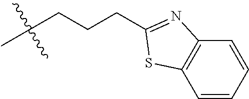 [SMILES: *CCCc1nc2ccccc2s1] | 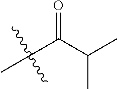 [SMILES: *C(=O)C(C)C] |
| 211 | 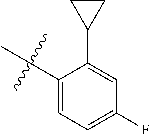 [SMILES: *c1ccc(F)cc1C1CC1] | 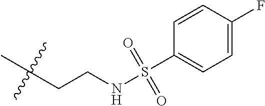 [SMILES: *CCNS(=O)(=O)c1ccc(F)cc1] | 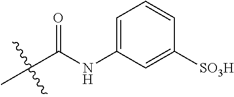 [SMILES: *C(=O)Nc1cccc(S(=O)(=O)O)c1] |
| 212 | 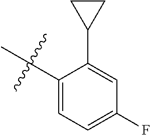 [SMILES: *c1ccc(F)cc1C1CC1] | 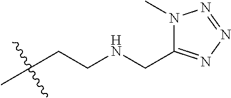 [SMILES: *CCNCc1nnnn1C] | 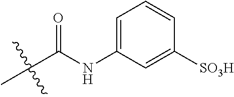 [SMILES: *C(=O)Nc1cccc(S(=O)(=O)O)c1] |
| 213 | 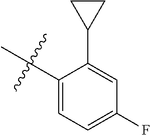 [SMILES: *c1ccc(F)cc1C1CC1] | 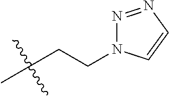 [SMILES: *CCn1ccnn1] | 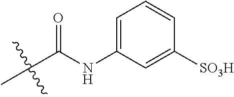 [SMILES: *C(=O)Nc1cccc(S(=O)(=O)O)c1] |
| 214 | 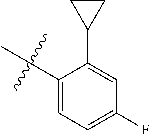 [SMILES: *c1ccc(F)cc1C1CC1] | 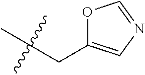 [SMILES: *Cc1cnco1] | 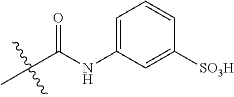 [SMILES: *C(=O)Nc1cccc(S(=O)(=O)O)c1] |
| 215 | 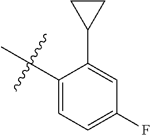 [SMILES: *c1ccc(F)cc1C1CC1] | 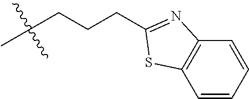 [SMILES: *CCCc1nc2ccccc2s1] | 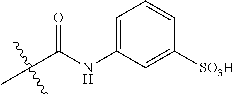 [SMILES: *C(=O)Nc1cccc(S(=O)(=O)O)c1] |
| 216 | 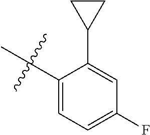 [SMILES: *c1ccc(F)cc1C1CC1] | 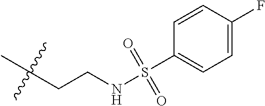 [SMILES: *CCNS(=O)(=O)c1ccc(F)cc1] | 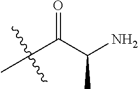 [SMILES: *C(=O)[C@H](C)N] |
| 217 | 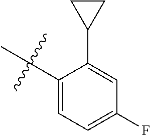 [SMILES: *c1ccc(F)cc1C1CC1] | 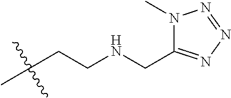 [SMILES: *CCNCc1nnnn1C] | 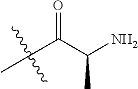 [SMILES: *C(=O)[C@H](C)N] |
| 218 | 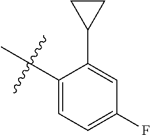 [SMILES: *c1ccc(F)cc1C1CC1] | 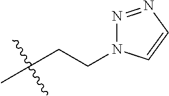 [SMILES: *CCn1ccnn1] | 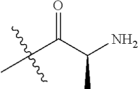 [SMILES: *C(=O)[C@H](C)N] |
| 219 | 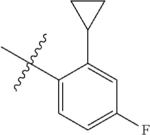 [SMILES: *c1ccc(F)cc1C1CC1] | 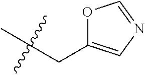 [SMILES: *Cc1cnco1] | 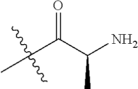 [SMILES: *C(=O)[C@H](C)N] |
| 220 | 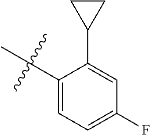 [SMILES: *c1ccc(F)cc1C1CC1] | 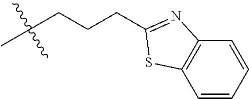 [SMILES: *CCCc1nc2ccccc2s1] | 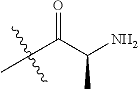 [SMILES: *C(=O)[C@H](C)N] |
| 221 | 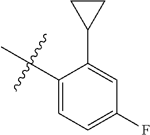 [SMILES: *c1ccc(F)cc1C1CC1] | 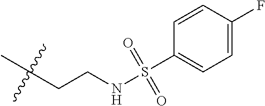 [SMILES: *CCNS(=O)(=O)c1ccc(F)cc1] | 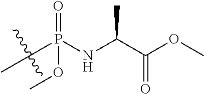 [SMILES: *P(=O)(N[C@@H](C)C(=O)OC)OC] |
| 222 | 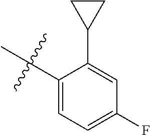 [SMILES: *c1ccc(F)cc1C1CC1] | 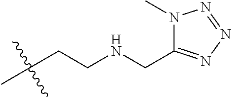 [SMILES: *CCNCc1nnnn1C] | 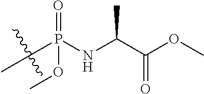 [SMILES: *P(=O)(N[C@@H](C)C(=O)OC)OC] |
| 223 | 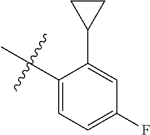 [SMILES: *c1ccc(F)cc1C1CC1] | 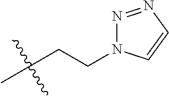 [SMILES: *CCn1ccnn1] | 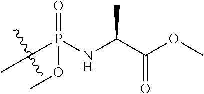 [SMILES: *P(=O)(N[C@@H](C)C(=O)OC)OC] |
| 224 | 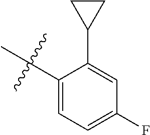 [SMILES: *c1ccc(F)cc1C1CC1] | 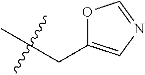 [SMILES: *Cc1cnco1] | 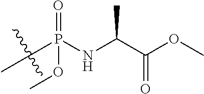 [SMILES: *P(=O)(N[C@@H](C)C(=O)OC)OC] |
| 225 | 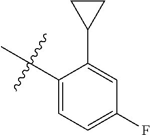 [SMILES: *c1ccc(F)cc1C1CC1] | 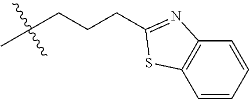 [SMILES: *CCCc1nc2ccccc2s1] | 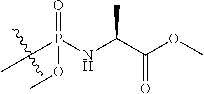 [SMILES: *P(=O)(N[C@@H](C)C(=O)OC)OC] |
| 226 | 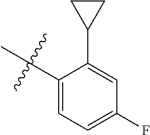 [SMILES: *c1ccc(F)cc1C1CC1] | 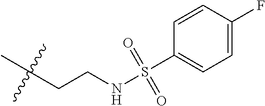 [SMILES: *CCNS(=O)(=O)c1ccc(F)cc1] | 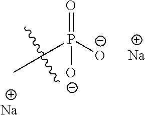 [SMILES: *P(=O)([O-])[O-] ; [Na+] ; [Na+]] |
| 227 | 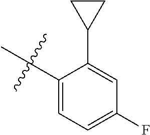 [SMILES: *c1ccc(F)cc1C1CC1] | 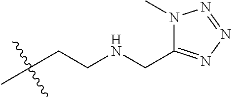 [SMILES: *CCNCc1nnnn1C] | 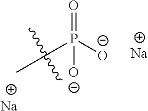 [SMILES: *P(=O)([O-])[O-] ; [Na+] ; [Na+]] |
| 228 | 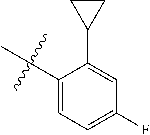 [SMILES: *c1ccc(F)cc1C1CC1] | 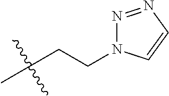 [SMILES: *CCn1ccnn1] | 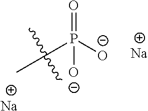 [SMILES: *P(=O)([O-])[O-] ; [Na+] ; [Na+]] |
| 229 | 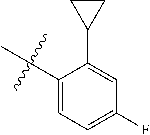 [SMILES: *c1ccc(F)cc1C1CC1] | 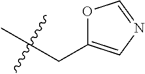 [SMILES: *Cc1cnco1] | 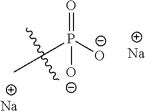 [SMILES: *P(=O)([O-])[O-] ; [Na+] ; [Na+]] |
| 230 | 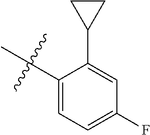 [SMILES: *c1ccc(F)cc1C1CC1] | 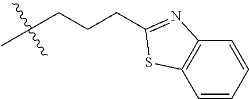 [SMILES: *CCCc1nc2ccccc2s1] | 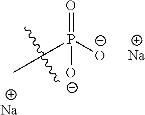 [SMILES: *P(=O)([O-])[O-] ; [Na+] ; [Na+]] |

Representative compounds of the invention include, but are not limited to, the following compounds (Entry 231 to Entry 518 in Table 4) according to Formula (XII),

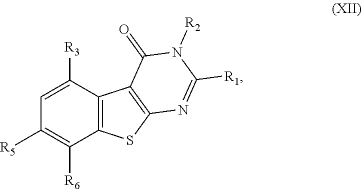

wherein R₃, R₅ and R₆ are delineated for each compound in Table 4.

TABLE 4

| Entry | R₃ | R₅ | R₆ |
|---|---|---|---|
| 231 | 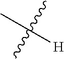 [SMILES: *[H]] | 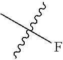 [SMILES: *F] | 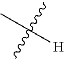 [SMILES: *[H]] |
| 232 | 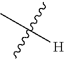 [SMILES: *[H]] | 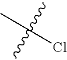 [SMILES: *Cl] | 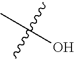 [SMILES: *O] |
| 233 | 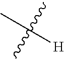 [SMILES: *[H]] | 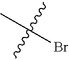 [SMILES: *Br] | 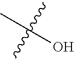 [SMILES: *O] |
| 234 | 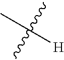 [SMILES: *[H]] | 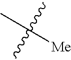 | 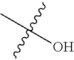 [SMILES: *O] |
| 235 | 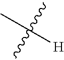 [SMILES: *[H]] | 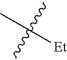 [SMILES: *CC] | 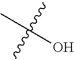 [SMILES: *O] |
| 236 | 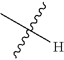 [SMILES: *[H]] | 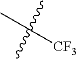 [SMILES: *C(F)(F)F] | 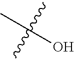 [SMILES: *O] |
| 237 | 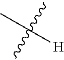 [SMILES: *[H]] | 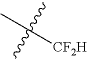 [SMILES: *C(F)F] | 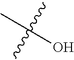 [SMILES: *O] |
| 238 | 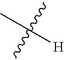 [SMILES: *[H]] | 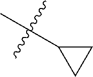 [SMILES: *C1CC1] | 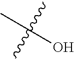 [SMILES: *O] |
| 239 | 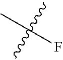 [SMILES: *F] | 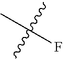 [SMILES: *F] | 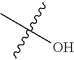 [SMILES: *O] |
| 240 | 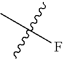 [SMILES: *F] | 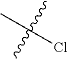 [SMILES: *Cl] | 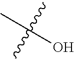 [SMILES: *O] |
| 241 | 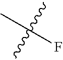 [SMILES: *F] | 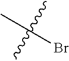 [SMILES: *Br] | 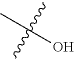 [SMILES: *O] |
| 242 | 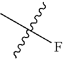 [SMILES: *F] | 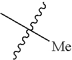 | 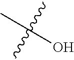 [SMILES: *O] |
| 243 | 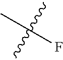 [SMILES: *F] | 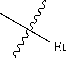 [SMILES: *CC] | 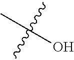 [SMILES: *O] |
| 244 | 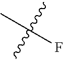 [SMILES: *F] | 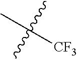 [SMILES: *C(F)(F)F] | 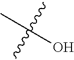 [SMILES: *O] |
| 245 | 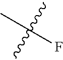 [SMILES: *F] | 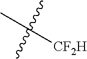 [SMILES: *C(F)F] | 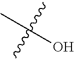 [SMILES: *O] |
| 246 | 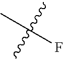 [SMILES: *F] | 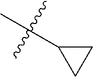 [SMILES: *C1CC1] | 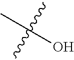 [SMILES: *O] |
| 247 | 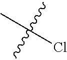 [SMILES: *Cl] | 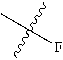 [SMILES: *F] | 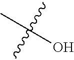 [SMILES: *O] |
| 248 | 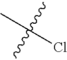 [SMILES: *Cl] | 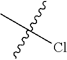 [SMILES: *Cl] | 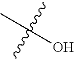 [SMILES: *O] |
| 249 | 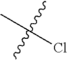 [SMILES: *Cl] | 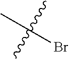 [SMILES: *Br] | 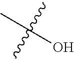 [SMILES: *O] |
| 250 | 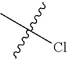 [SMILES: *Cl] | 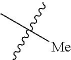 | 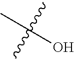 [SMILES: *O] |
| 251 | 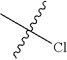 [SMILES: *Cl] | 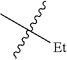 [SMILES: *CC] | 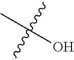 [SMILES: *O] |
| 252 | 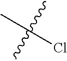 [SMILES: *Cl] | 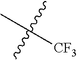 [SMILES: *C(F)(F)F] | 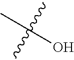 [SMILES: *O] |
| 253 | 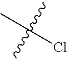 [SMILES: *Cl] | 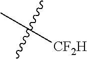 [SMILES: *C(F)F] | 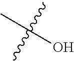 [SMILES: *O] |
| 254 | 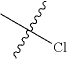 [SMILES: *Cl] | 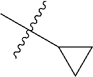 [SMILES: *C1CC1] | 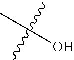 [SMILES: *O] |
| 255 | 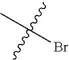 [SMILES: *Br] | 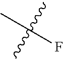 [SMILES: *F] | 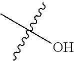 [SMILES: *O] |
| 256 | 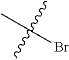 [SMILES: *Br] | 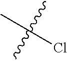 [SMILES: *Cl] | 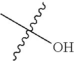 [SMILES: *O] |
| 257 | 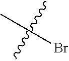 [SMILES: *Br] | 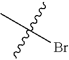 [SMILES: *Br] | 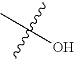 [SMILES: *O] |
| 258 | 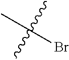 [SMILES: *Br] | 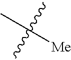 | 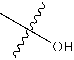 [SMILES: *O] |
| 259 | 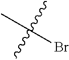 [SMILES: *Br] | 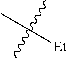 [SMILES: *CC] | 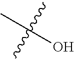 [SMILES: *O] |
| 260 | 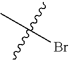 [SMILES: *Br] | 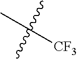 [SMILES: *C(F)(F)F] | 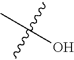 [SMILES: *O] |
| 261 | 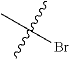 [SMILES: *Br] | 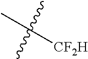 [SMILES: *C(F)F] | 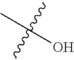 [SMILES: *O] |
| 262 | 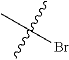 [SMILES: *Br] | 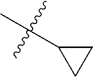 [SMILES: *C1CC1] | 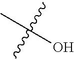 [SMILES: *O] |
| 263 | 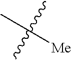 | 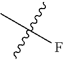 [SMILES: *F] | 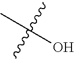 [SMILES: *O] |
| 264 | 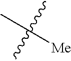 | 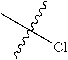 [SMILES: *Cl] | 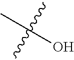 [SMILES: *O] |
| 265 | 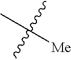 | 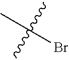 [SMILES: *Br] | 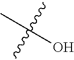 [SMILES: *O] |
| 266 | 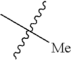 | 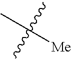 | 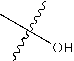 [SMILES: *O] |
| 267 | 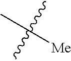 | 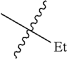 [SMILES: *CC] | 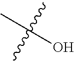 [SMILES: *O] |
| 268 | 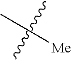 | 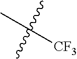 [SMILES: *C(F)(F)F] | 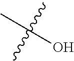 [SMILES: *O] |
| 269 | 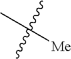 | 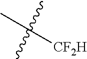 [SMILES: *C(F)F] | 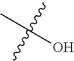 [SMILES: *O] |
| 270 | 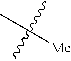 | 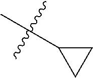 [SMILES: *C1CC1] | 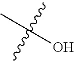 [SMILES: *O] |
| 271 | 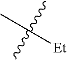 [SMILES: *CC] | 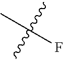 [SMILES: *F] | 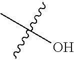 [SMILES: *O] |
| 272 | 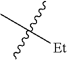 [SMILES: *CC] | 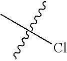 [SMILES: *Cl] | 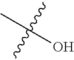 [SMILES: *O] |
| 273 | 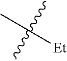 [SMILES: *CC] | 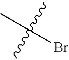 [SMILES: *Br] | 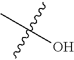 [SMILES: *O] |
| 274 | 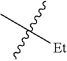 [SMILES: *CC] | 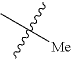 | 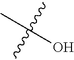 [SMILES: *O] |
| 275 | 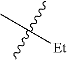 [SMILES: *CC] | 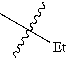 [SMILES: *CC] | 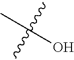 [SMILES: *O] |
| 276 | 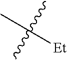 [SMILES: *CC] | 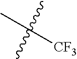 [SMILES: *C(F)(F)F] | 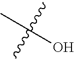 [SMILES: *O] |
| 277 | 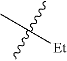 [SMILES: *CC] | 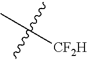 [SMILES: *C(F)F] | 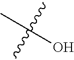 [SMILES: *O] |
| 278 | 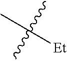 [SMILES: *CC] | 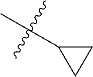 [SMILES: *C1CC1] | 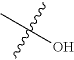 [SMILES: *O] |
| 279 | 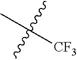 [SMILES: *C(F)(F)F] | 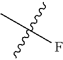 [SMILES: *F] | 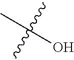 [SMILES: *O] |
| 280 | 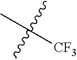 [SMILES: *C(F)(F)F] | 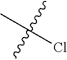 [SMILES: *Cl] | 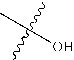 [SMILES: *O] |
| 281 | 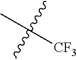 [SMILES: *C(F)(F)F] | 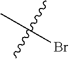 [SMILES: *Br] | 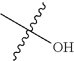 [SMILES: *O] |
| 282 | 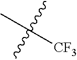 [SMILES: *C(F)(F)F] | 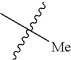 | 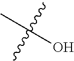 [SMILES: *O] |
| 283 | 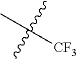 [SMILES: *C(F)(F)F] | 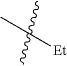 [SMILES: *CC] | 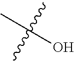 [SMILES: *O] |
| 284 | 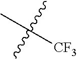 [SMILES: *C(F)(F)F] | 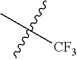 [SMILES: *C(F)(F)F] | 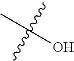 [SMILES: *O] |
| 285 | 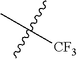 [SMILES: *C(F)(F)F] | 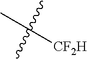 [SMILES: *C(F)F] | 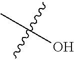 [SMILES: *O] |
| 286 | 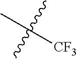 [SMILES: *C(F)(F)F] | 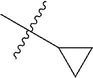 [SMILES: *C1CC1] | 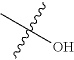 [SMILES: *O] |
| 287 | 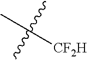 [SMILES: *C(F)F] | 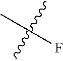 [SMILES: *F] | 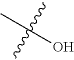 [SMILES: *O] |
| 288 | 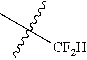 [SMILES: *C(F)F] | 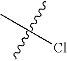 [SMILES: *Cl] | 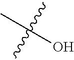 [SMILES: *O] |
| 289 | 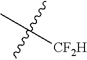 [SMILES: *C(F)F] | 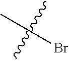 [SMILES: *Br] | 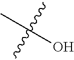 [SMILES: *O] |
| 290 | 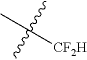 [SMILES: *C(F)F] | 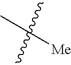 | 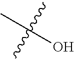 [SMILES: *O] |
| 291 | 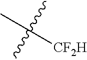 [SMILES: *C(F)F] | 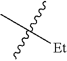 [SMILES: *CC] | 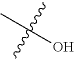 [SMILES: *O] |
| 292 | 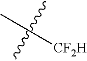 [SMILES: *C(F)F] | 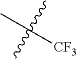 [SMILES: *C(F)(F)F] | 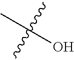 [SMILES: *O] |
| 293 | 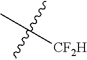 [SMILES: *C(F)F] | 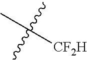 [SMILES: *C(F)F] | 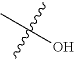 [SMILES: *O] |
| 294 | 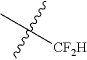 [SMILES: *C(F)F] | 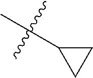 [SMILES: *C1CC1] | 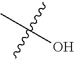 [SMILES: *O] |
| 295 | 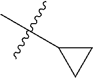 [SMILES: *C1CC1] | 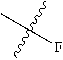 [SMILES: *F] | 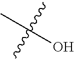 [SMILES: *O] |
| 296 | 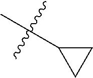 [SMILES: *C1CC1] | 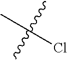 [SMILES: *Cl] | 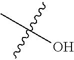 [SMILES: *O] |
| 297 | 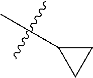 [SMILES: *C1CC1] | 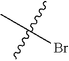 [SMILES: *Br] | 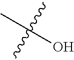 [SMILES: *O] |
| 298 | 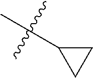 [SMILES: *C1CC1] | 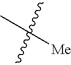 | 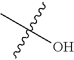 [SMILES: *O] |
| 299 | 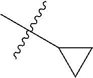 [SMILES: *C1CC1] | 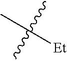 [SMILES: *CC] | 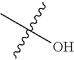 [SMILES: *O] |
| 300 | 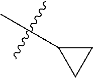 [SMILES: *C1CC1] | 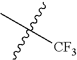 [SMILES: *C(F)(F)F] | 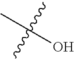 [SMILES: *O] |
| 301 | 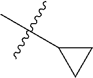 [SMILES: *C1CC1] | 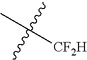 [SMILES: *C(F)F] | 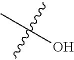 [SMILES: *O] |
| 302 | 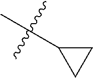 [SMILES: *C1CC1] | 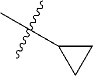 [SMILES: *C1CC1] | 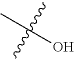 [SMILES: *O] |
| 303 | 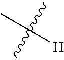 [SMILES: *[H]] | 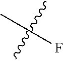 [SMILES: *F] | 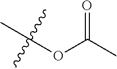 [SMILES: *OC(C)=O] |
| 304 | 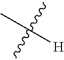 [SMILES: *[H]] | 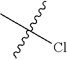 [SMILES: *Cl] | 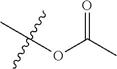 [SMILES: *OC(C)=O] |
| 305 | 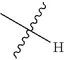 [SMILES: *[H]] | 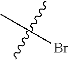 [SMILES: *Br] | 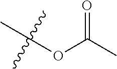 [SMILES: *OC(C)=O] |
| 306 | 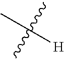 [SMILES: *[H]] | 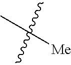 | 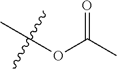 [SMILES: *OC(C)=O] |
| 307 | 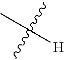 [SMILES: *[H]] | 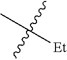 [SMILES: *CC] | 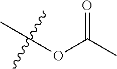 [SMILES: *OC(C)=O] |
| 308 | 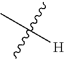 [SMILES: *[H]] | 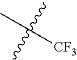 [SMILES: *C(F)(F)F] | 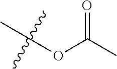 [SMILES: *OC(C)=O] |
| 309 | 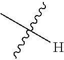 [SMILES: *[H]] | 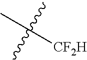 [SMILES: *C(F)F] | 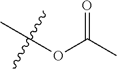 [SMILES: *OC(C)=O] |
| 311 | 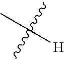 [SMILES: *[H]] | 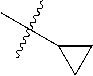 [SMILES: *C1CC1] | 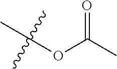 [SMILES: *OC(C)=O] |
| 312 | 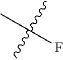 [SMILES: *F] | 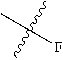 [SMILES: *F] | 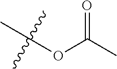 [SMILES: *OC(C)=O] |
| 313 | 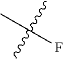 [SMILES: *F] | 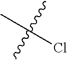 [SMILES: *Cl] | 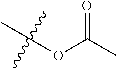 [SMILES: *OC(C)=O] |
| 314 | 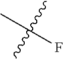 [SMILES: *F] | 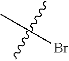 [SMILES: *Br] | 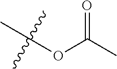 [SMILES: *OC(C)=O] |
| 315 | 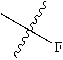 [SMILES: *F] | 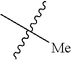 | 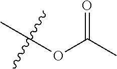 [SMILES: *OC(C)=O] |
| 316 | 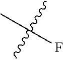 [SMILES: *F] | 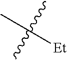 [SMILES: *CC] | 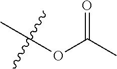 [SMILES: *OC(C)=O] |
| 317 | 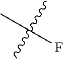 [SMILES: *F] | 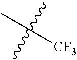 [SMILES: *C(F)(F)F] | 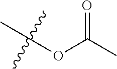 [SMILES: *OC(C)=O] |
| 318 | 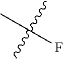 [SMILES: *F] | 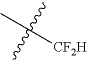 [SMILES: *C(F)F] | 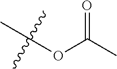 [SMILES: *OC(C)=O] |
| 319 | 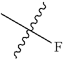 [SMILES: *F] | 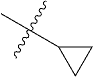 [SMILES: *C1CC1] | 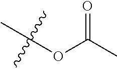 [SMILES: *OC(C)=O] |
| 320 | 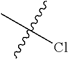 [SMILES: *Cl] | 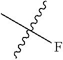 [SMILES: *F] | 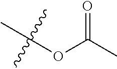 [SMILES: *OC(C)=O] |
| 321 | 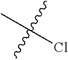 [SMILES: *Cl] | 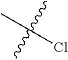 [SMILES: *Cl] | 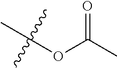 [SMILES: *OC(C)=O] |
| 322 | 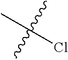 [SMILES: *Cl] | 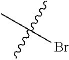 [SMILES: *Br] | 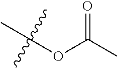 [SMILES: *OC(C)=O] |
| 323 | 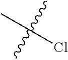 [SMILES: *Cl] | 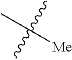 | 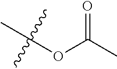 [SMILES: *OC(C)=O] |
| 324 | 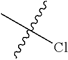 [SMILES: *Cl] | 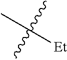 [SMILES: *CC] | 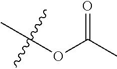 [SMILES: *OC(C)=O] |
| 325 | 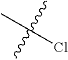 [SMILES: *Cl] | 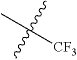 [SMILES: *C(F)(F)F] | 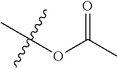 [SMILES: *OC(C)=O] |
| 326 | 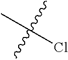 [SMILES: *Cl] | 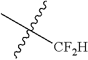 [SMILES: *C(F)F] | 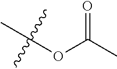 [SMILES: *OC(C)=O] |
| 327 | 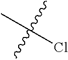 [SMILES: *Cl] | 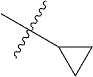 [SMILES: *C1CC1] | 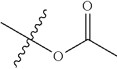 [SMILES: *OC(C)=O] |
| 328 | 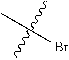 [SMILES: *Br] | 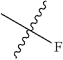 [SMILES: *F] | 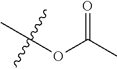 [SMILES: *OC(C)=O] |
| 329 | 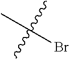 [SMILES: *Br] | 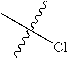 [SMILES: *Cl] | 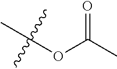 [SMILES: *OC(C)=O] |
| 330 | 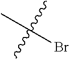 [SMILES: *Br] | 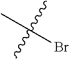 [SMILES: *Br] | 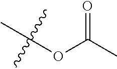 [SMILES: *OC(C)=O] |
| 331 | 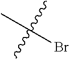 [SMILES: *Br] | 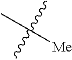 | 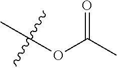 [SMILES: *OC(C)=O] |
| 332 | 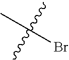 [SMILES: *Br] | 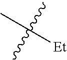 [SMILES: *CC] | 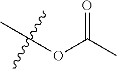 [SMILES: *OC(C)=O] |
| 333 | 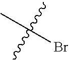 [SMILES: *Br] | 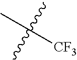 [SMILES: *C(F)(F)F] | 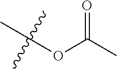 [SMILES: *OC(C)=O] |
| 330 | 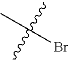 [SMILES: *Br] | 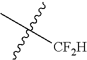 [SMILES: *C(F)F] | 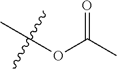 [SMILES: *OC(C)=O] |
| 334 | 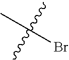 [SMILES: *Br] | 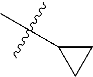 [SMILES: *C1CC1] | 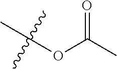 [SMILES: *OC(C)=O] |
| 335 | 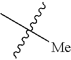 | 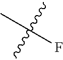 [SMILES: *F] | 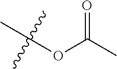 [SMILES: *OC(C)=O] |
| 336 | 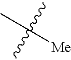 | 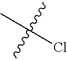 [SMILES: *Cl] | 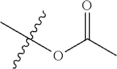 [SMILES: *OC(C)=O] |
| 337 | 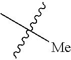 | 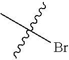 [SMILES: *Br] | 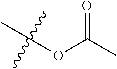 [SMILES: *OC(C)=O] |
| 338 | 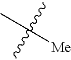 | 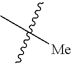 | 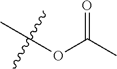 [SMILES: *OC(C)=O] |
| 339 | 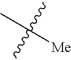 | 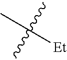 [SMILES: *CC] | 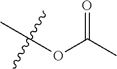 [SMILES: *OC(C)=O] |
| 340 | 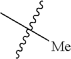 | 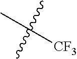 [SMILES: *C(F)(F)F] | 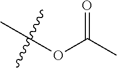 [SMILES: *OC(C)=O] |
| 341 | 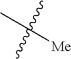 | 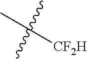 [SMILES: *C(F)F] | 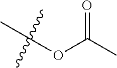 [SMILES: *OC(C)=O] |
| 342 | 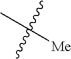 | 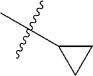 [SMILES: *C1CC1] | 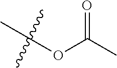 [SMILES: *OC(C)=O] |
| 343 | 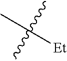 [SMILES: *CC] | 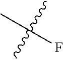 [SMILES: *F] | 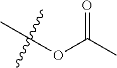 [SMILES: *OC(C)=O] |
| 344 | 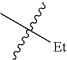 [SMILES: *CC] | 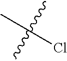 [SMILES: *Cl] | 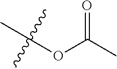 [SMILES: *OC(C)=O] |
| 345 | 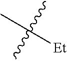 [SMILES: *CC] | 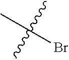 [SMILES: *Br] | 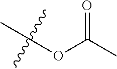 [SMILES: *OC(C)=O] |
| 346 | 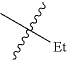 [SMILES: *CC] | 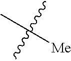 | 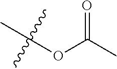 [SMILES: *OC(C)=O] |
| 347 | 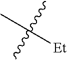 [SMILES: *CC] | 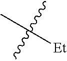 [SMILES: *CC] | 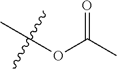 [SMILES: *OC(C)=O] |
| 348 | 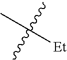 [SMILES: *CC] | 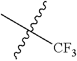 [SMILES: *C(F)(F)F] | 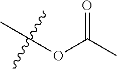 [SMILES: *OC(C)=O] |
| 349 | 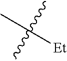 [SMILES: *CC] | 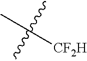 [SMILES: *C(F)F] | 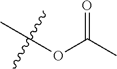 [SMILES: *OC(C)=O] |
| 350 | 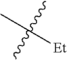 [SMILES: *CC] | 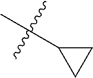 [SMILES: *C1CC1] | 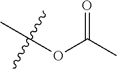 [SMILES: *OC(C)=O] |
| 351 | 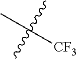 [SMILES: *C(F)(F)F] | 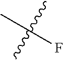 [SMILES: *F] | 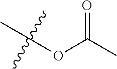 [SMILES: *OC(C)=O] |
| 352 | 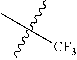 [SMILES: *C(F)(F)F] | 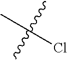 [SMILES: *Cl] | 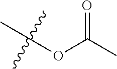 [SMILES: *OC(C)=O] |
| 353 | 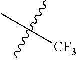 [SMILES: *C(F)(F)F] | 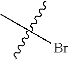 [SMILES: *Br] | 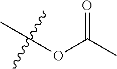 [SMILES: *OC(C)=O] |
| 354 | 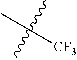 [SMILES: *C(F)(F)F] | 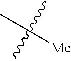 | 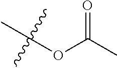 [SMILES: *OC(C)=O] |
| 355 | 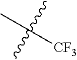 [SMILES: *C(F)(F)F] | 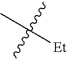 [SMILES: *CC] | 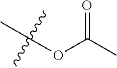 [SMILES: *OC(C)=O] |
| 356 | 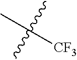 [SMILES: *C(F)(F)F] | 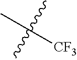 [SMILES: *C(F)(F)F] | 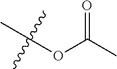 [SMILES: *OC(C)=O] |
| 357 | 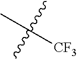 [SMILES: *C(F)(F)F] | 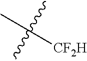 [SMILES: *C(F)F] | 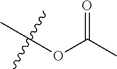 [SMILES: *OC(C)=O] |
| 358 | 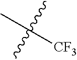 [SMILES: *C(F)(F)F] | 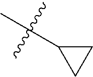 [SMILES: *C1CC1] | 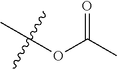 [SMILES: *OC(C)=O] |
| 359 | 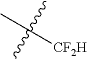 [SMILES: *C(F)F] | 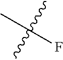 [SMILES: *F] | 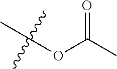 [SMILES: *OC(C)=O] |
| 360 | 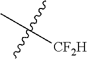 [SMILES: *C(F)F] | 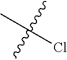 [SMILES: *Cl] | 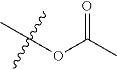 [SMILES: *OC(C)=O] |
| 361 | 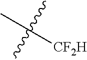 [SMILES: *C(F)F] | 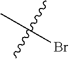 [SMILES: *Br] | 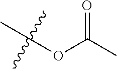 [SMILES: *OC(C)=O] |
| 362 | 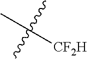 [SMILES: *C(F)F] | 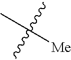 | 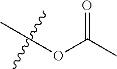 [SMILES: *OC(C)=O] |
| 363 | 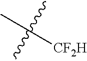 [SMILES: *C(F)F] | 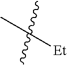 [SMILES: *CC] | 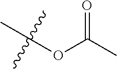 [SMILES: *OC(C)=O] |
| 364 | 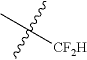 [SMILES: *C(F)F] | 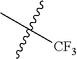 [SMILES: *C(F)(F)F] | 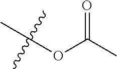 [SMILES: *OC(C)=O] |
| 365 | 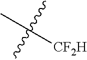 [SMILES: *C(F)F] | 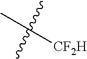 [SMILES: *C(F)F] | 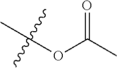 [SMILES: *OC(C)=O] |
| 366 | 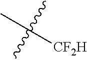 [SMILES: *C(F)F] | 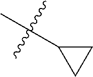 [SMILES: *C1CC1] | 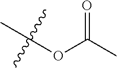 [SMILES: *OC(C)=O] |
| 367 | 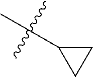 [SMILES: *C1CC1] | 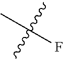 [SMILES: *F] | 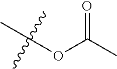 [SMILES: *OC(C)=O] |
| 368 | 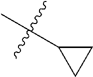 [SMILES: *C1CC1] | 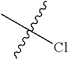 [SMILES: *Cl] | 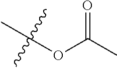 [SMILES: *OC(C)=O] |
| 369 | 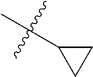 [SMILES: *C1CC1] | 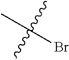 [SMILES: *Br] | 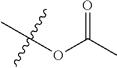 [SMILES: *OC(C)=O] |
| 370 | 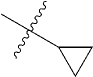 [SMILES: *C1CC1] | 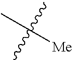 | 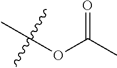 [SMILES: *OC(C)=O] |
| 371 | 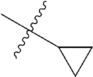 [SMILES: *C1CC1] | 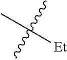 [SMILES: *CC] | 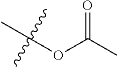 [SMILES: *OC(C)=O] |
| 372 | 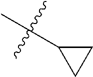 [SMILES: *C1CC1] | 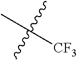 [SMILES: *C(F)(F)F] | 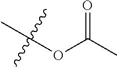 [SMILES: *OC(C)=O] |
| 373 | 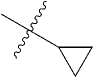 [SMILES: *C1CC1] | 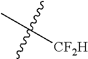 [SMILES: *C(F)F] | 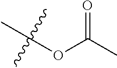 [SMILES: *OC(C)=O] |
| 374 | 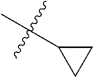 [SMILES: *C1CC1] | 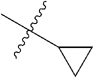 [SMILES: *C1CC1] | 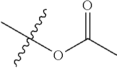 [SMILES: *OC(C)=O] |
| 375 | 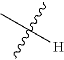 [SMILES: *[H]] | 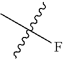 [SMILES: *F] | 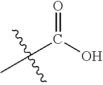 [SMILES: *C(=O)O] |
| 376 | 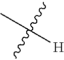 [SMILES: *[H]] | 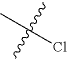 [SMILES: *Cl] | 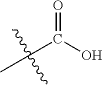 [SMILES: *C(=O)O] |
| 377 | 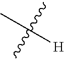 [SMILES: *[H]] | 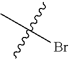 [SMILES: *Br] | 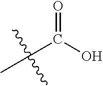 [SMILES: *C(=O)O] |
| 378 | 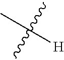 [SMILES: *[H]] | 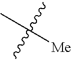 | 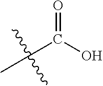 [SMILES: *C(=O)O] |
| 379 | 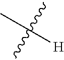 [SMILES: *[H]] | 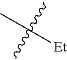 [SMILES: *CC] | 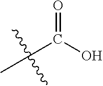 [SMILES: *C(=O)O] |
| 380 | 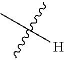 [SMILES: *[H]] | 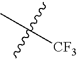 [SMILES: *C(F)(F)F] | 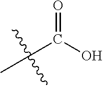 [SMILES: *C(=O)O] |
| 381 | 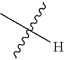 [SMILES: *[H]] | 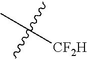 [SMILES: *C(F)F] | 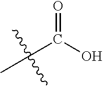 [SMILES: *C(=O)O] |
| 382 | 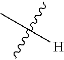 [SMILES: *[H]] | 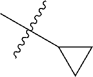 [SMILES: *C1CC1] | 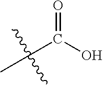 [SMILES: *C(=O)O] |
| 383 | 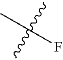 [SMILES: *F] | 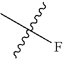 [SMILES: *F] | 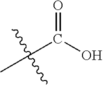 [SMILES: *C(=O)O] |
| 384 | 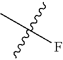 [SMILES: *F] | 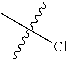 [SMILES: *Cl] | 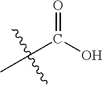 [SMILES: *C(=O)O] |
| 385 | 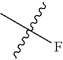 [SMILES: *F] | 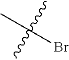 [SMILES: *Br] | 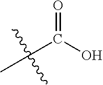 [SMILES: *C(=O)O] |
| 386 | 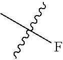 [SMILES: *F] | 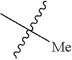 | 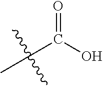 [SMILES: *C(=O)O] |
| 387 | 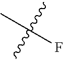 [SMILES: *F] | 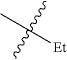 [SMILES: *CC] | 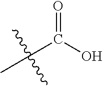 [SMILES: *C(=O)O] |
| 388 | 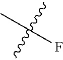 [SMILES: *F] | 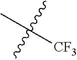 [SMILES: *C(F)(F)F] | 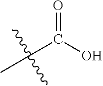 [SMILES: *C(=O)O] |
| 389 | 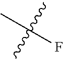 [SMILES: *F] | 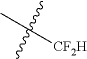 [SMILES: *C(F)F] | 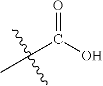 [SMILES: *C(=O)O] |
| 390 | 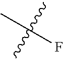 [SMILES: *F] | 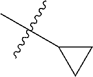 [SMILES: *C1CC1] | 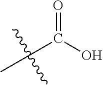 [SMILES: *C(=O)O] |
| 391 | 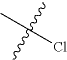 [SMILES: *Cl] | 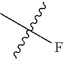 [SMILES: *F] | 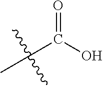 [SMILES: *C(=O)O] |
| 392 | 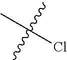 [SMILES: *Cl] | 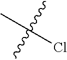 [SMILES: *Cl] | 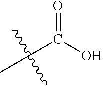 [SMILES: *C(=O)O] |
| 393 | 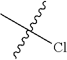 [SMILES: *Cl] | 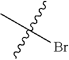 [SMILES: *Br] | 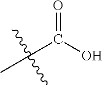 [SMILES: *C(=O)O] |
| 394 | 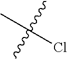 [SMILES: *Cl] | 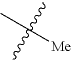 | 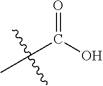 [SMILES: *C(=O)O] |
| 395 | 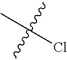 [SMILES: *Cl] | 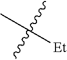 [SMILES: *CC] | 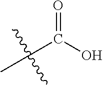 [SMILES: *C(=O)O] |
| 396 | 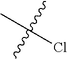 [SMILES: *Cl] | 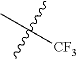 [SMILES: *C(F)(F)F] | 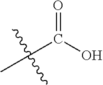 [SMILES: *C(=O)O] |
| 397 | 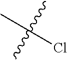 [SMILES: *Cl] | 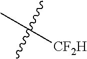 [SMILES: *C(F)F] | 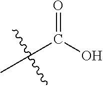 [SMILES: *C(=O)O] |
| 398 | 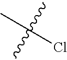 [SMILES: *Cl] | 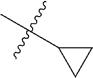 [SMILES: *C1CC1] | 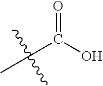 [SMILES: *C(=O)O] |
| 399 | 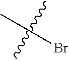 [SMILES: *Br] | 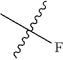 [SMILES: *F] | 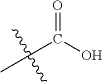 [SMILES: *C(=O)O] |
| 400 | 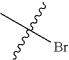 [SMILES: *Br] | 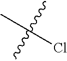 [SMILES: *Cl] | 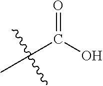 [SMILES: *C(=O)O] |
| 401 | 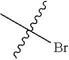 [SMILES: *Br] | 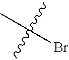 [SMILES: *Br] | 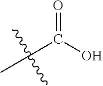 [SMILES: *C(=O)O] |
| 402 | 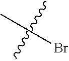 [SMILES: *Br] | 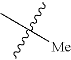 | 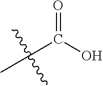 [SMILES: *C(=O)O] |
| 403 | 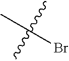 [SMILES: *Br] | 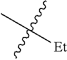 [SMILES: *CC] | 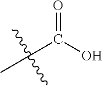 [SMILES: *C(=O)O] |
| 404 | 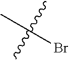 [SMILES: *Br] | 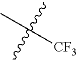 [SMILES: *C(F)(F)F] | 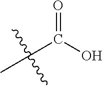 [SMILES: *C(=O)O] |
| 405 | 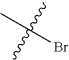 [SMILES: *Br] | 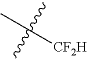 [SMILES: *C(F)F] | 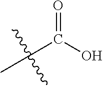 [SMILES: *C(=O)O] |
| 406 | 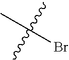 [SMILES: *Br] | 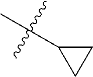 [SMILES: *C1CC1] | 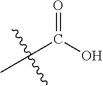 [SMILES: *C(=O)O] |
| 407 | 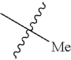 | 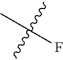 [SMILES: *F] | 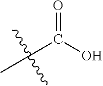 [SMILES: *C(=O)O] |
| 408 | 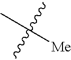 | 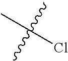 [SMILES: *Cl] | 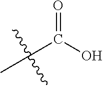 [SMILES: *C(=O)O] |
| 409 | 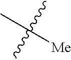 | 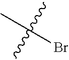 [SMILES: *Br] | 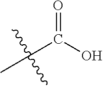 [SMILES: *C(=O)O] |
| 410 | 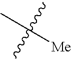 | 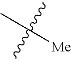 | 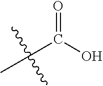 [SMILES: *C(=O)O] |
| 411 | 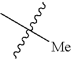 | 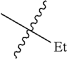 [SMILES: *CC] | 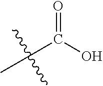 [SMILES: *C(=O)O] |
| 412 | 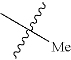 | 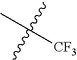 [SMILES: *C(F)(F)F] | 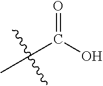 [SMILES: *C(=O)O] |
| 413 | 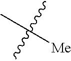 | 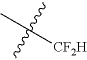 [SMILES: *C(F)F] | 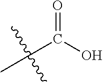 [SMILES: *C(=O)O] |
| 414 | 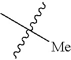 | 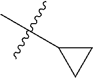 [SMILES: *C1CC1] | 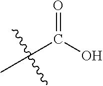 [SMILES: *C(=O)O] |
| 415 | 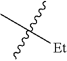 [SMILES: *CC] | 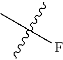 [SMILES: *F] | 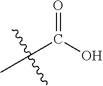 [SMILES: *C(=O)O] |
| 416 | 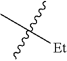 [SMILES: *CC] | 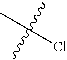 [SMILES: *Cl] | 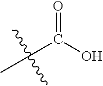 [SMILES: *C(=O)O] |
| 417 | 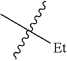 [SMILES: *CC] | 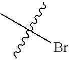 [SMILES: *Br] | 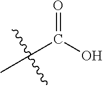 [SMILES: *C(=O)O] |
| 418 | 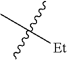 [SMILES: *CC] | 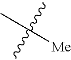 | 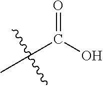 [SMILES: *C(=O)O] |
| 419 | 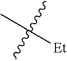 [SMILES: *CC] | 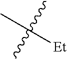 [SMILES: *CC] | 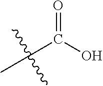 [SMILES: *C(=O)O] |
| 420 | 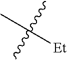 [SMILES: *CC] | 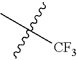 [SMILES: *C(F)(F)F] | 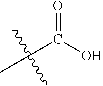 [SMILES: *C(=O)O] |
| 421 | 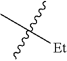 [SMILES: *CC] | 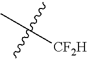 [SMILES: *C(F)F] | 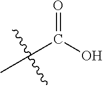 [SMILES: *C(=O)O] |
| 422 | 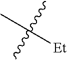 [SMILES: *CC] | 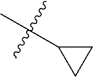 [SMILES: *C1CC1] | 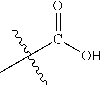 [SMILES: *C(=O)O] |
| 423 | 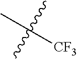 [SMILES: *C(F)(F)F] | 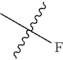 [SMILES: *F] | 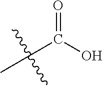 [SMILES: *C(=O)O] |
| 424 | 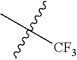 [SMILES: *C(F)(F)F] | 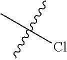 [SMILES: *Cl] | 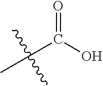 [SMILES: *C(=O)O] |
| 425 | 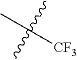 [SMILES: *C(F)(F)F] | 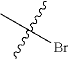 [SMILES: *Br] | 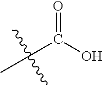 [SMILES: *C(=O)O] |
| 426 | 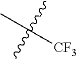 [SMILES: *C(F)(F)F] | 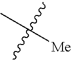 | 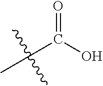 [SMILES: *C(=O)O] |
| 427 | 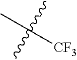 [SMILES: *C(F)(F)F] | 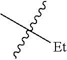 [SMILES: *CC] | 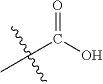 [SMILES: *C(=O)O] |
| 428 | 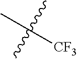 [SMILES: *C(F)(F)F] | 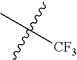 [SMILES: *C(F)(F)F] | 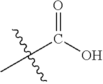 [SMILES: *C(=O)O] |
| 429 | 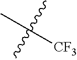 [SMILES: *C(F)(F)F] | 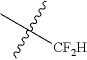 [SMILES: *C(F)F] | 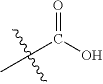 [SMILES: *C(=O)O] |
| 430 | 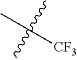 [SMILES: *C(F)(F)F] | 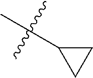 [SMILES: *C1CC1] | 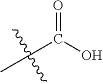 [SMILES: *C(=O)O] |
| 431 | 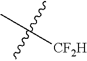 [SMILES: *C(F)F] | 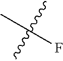 [SMILES: *F] | 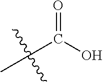 [SMILES: *C(=O)O] |
| 432 | 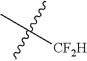 [SMILES: *C(F)F] | 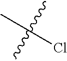 [SMILES: *Cl] | 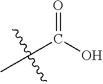 [SMILES: *C(=O)O] |
| 433 | 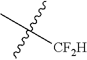 [SMILES: *C(F)F] | 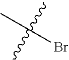 [SMILES: *Br] | 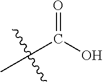 [SMILES: *C(=O)O] |
| 434 | 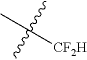 [SMILES: *C(F)F] | 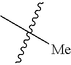 | 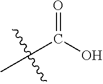 [SMILES: *C(=O)O] |
| 435 | 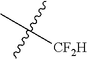 [SMILES: *C(F)F] | 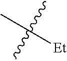 [SMILES: *CC] | 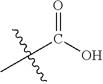 [SMILES: *C(=O)O] |
| 436 | 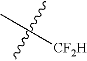 [SMILES: *C(F)F] | 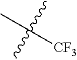 [SMILES: *C(F)(F)F] | 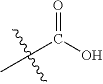 [SMILES: *C(=O)O] |
| 437 | 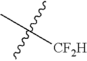 [SMILES: *C(F)F] | 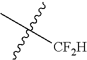 [SMILES: *C(F)F] | 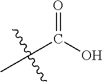 [SMILES: *C(=O)O] |
| 438 | 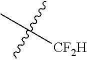 [SMILES: *C(F)F] | 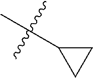 [SMILES: *C1CC1] | 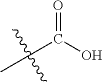 [SMILES: *C(=O)O] |
| 439 | 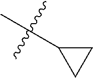 [SMILES: *C1CC1] | 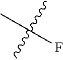 [SMILES: *F] | 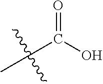 [SMILES: *C(=O)O] |
| 440 | 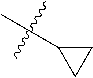 [SMILES: *C1CC1] | 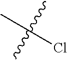 [SMILES: *Cl] | 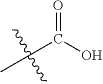 [SMILES: *C(=O)O] |
| 441 | 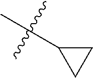 [SMILES: *C1CC1] | 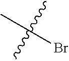 [SMILES: *Br] | 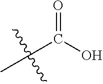 [SMILES: *C(=O)O] |
| 442 | 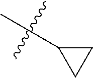 [SMILES: *C1CC1] | 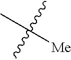 | 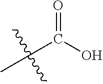 [SMILES: *C(=O)O] |
| 443 | 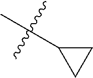 [SMILES: *C1CC1] | 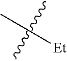 [SMILES: *CC] | 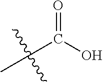 [SMILES: *C(=O)O] |
| 444 | 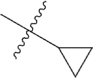 [SMILES: *C1CC1] | 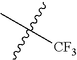 [SMILES: *C(F)(F)F] | 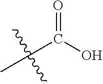 [SMILES: *C(=O)O] |
| 445 | 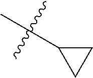 [SMILES: *C1CC1] | 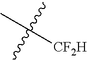 [SMILES: *C(F)F] | 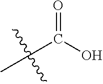 [SMILES: *C(=O)O] |
| 446 | 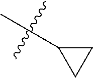 [SMILES: *C1CC1] | 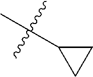 [SMILES: *C1CC1] | 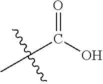 [SMILES: *C(=O)O] |
| 447 | 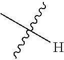 [SMILES: *[H]] | 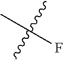 [SMILES: *F] | 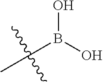 [SMILES: *B(O)O] |
| 448 | 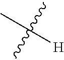 [SMILES: *[H]] | 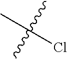 [SMILES: *Cl] | 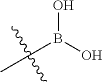 [SMILES: *B(O)O] |
| 449 | 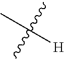 [SMILES: *[H]] | 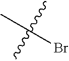 [SMILES: *Br] | 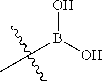 [SMILES: *B(O)O] |
| 450 | 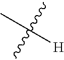 [SMILES: *[H]] | 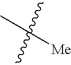 | 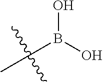 [SMILES: *B(O)O] |
| 451 | 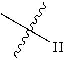 [SMILES: *[H]] | 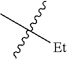 [SMILES: *CC] | 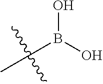 [SMILES: *B(O)O] |
| 452 | 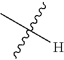 [SMILES: *[H]] | 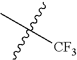 [SMILES: *C(F)(F)F] | 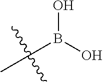 [SMILES: *B(O)O] |
| 453 | 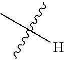 [SMILES: *[H]] | 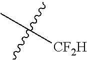 [SMILES: *C(F)F] | 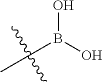 [SMILES: *B(O)O] |
| 454 | 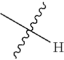 [SMILES: *[H]] | 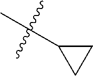 [SMILES: *C1CC1] | 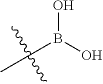 [SMILES: *B(O)O] |
| 455 | 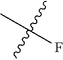 [SMILES: *F] | 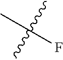 [SMILES: *F] | 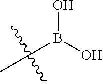 [SMILES: *B(O)O] |
| 456 | 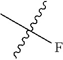 [SMILES: *F] | 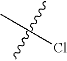 [SMILES: *Cl] | 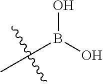 [SMILES: *B(O)O] |
| 457 | 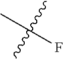 [SMILES: *F] | 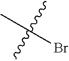 [SMILES: *Br] | 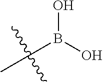 [SMILES: *B(O)O] |
| 458 | 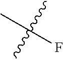 [SMILES: *F] | 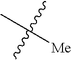 | 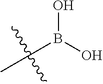 [SMILES: *B(O)O] |
| 459 | 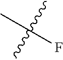 [SMILES: *F] | 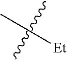 [SMILES: *CC] | 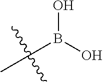 [SMILES: *B(O)O] |
| 460 | 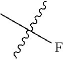 [SMILES: *F] | 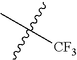 [SMILES: *C(F)(F)F] | 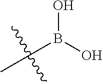 [SMILES: *B(O)O] |
| 461 | 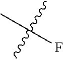 [SMILES: *F] | 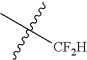 [SMILES: *C(F)F] | 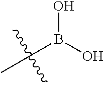 [SMILES: *B(O)O] |
| 462 | 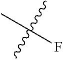 [SMILES: *F] | 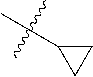 [SMILES: *C1CC1] | 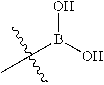 [SMILES: *B(O)O] |
| 463 | 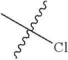 [SMILES: *Cl] | 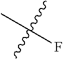 [SMILES: *F] | 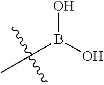 [SMILES: *B(O)O] |
| 464 | 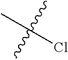 [SMILES: *Cl] | 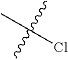 [SMILES: *Cl] | 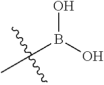 [SMILES: *B(O)O] |
| 465 | 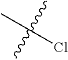 [SMILES: *Cl] | 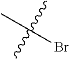 [SMILES: *Br] | 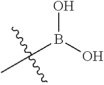 [SMILES: *B(O)O] |
| 466 | 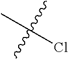 [SMILES: *Cl] | 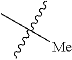 | 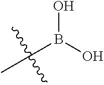 [SMILES: *B(O)O] |
| 467 | 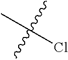 [SMILES: *Cl] | 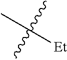 [SMILES: *CC] | 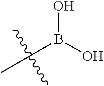 [SMILES: *B(O)O] |
| 468 | 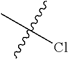 [SMILES: *Cl] | 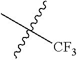 [SMILES: *C(F)(F)F] | 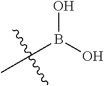 [SMILES: *B(O)O] |
| 469 | 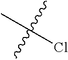 [SMILES: *Cl] | 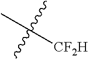 [SMILES: *C(F)F] | 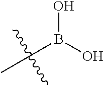 [SMILES: *B(O)O] |
| 470 | 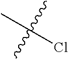 [SMILES: *Cl] | 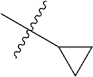 [SMILES: *C1CC1] | 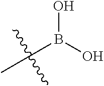 [SMILES: *B(O)O] |
| 471 | 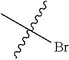 [SMILES: *Br] | 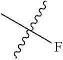 [SMILES: *F] | 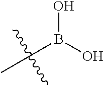 [SMILES: *B(O)O] |
| 472 | 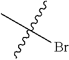 [SMILES: *Br] | 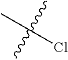 [SMILES: *Cl] | 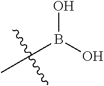 [SMILES: *B(O)O] |
| 473 | 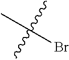 [SMILES: *Br] | 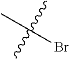 [SMILES: *Br] | 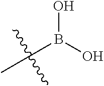 [SMILES: *B(O)O] |
| 474 | 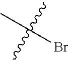 [SMILES: *Br] | 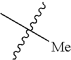 | 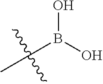 [SMILES: *B(O)O] |
| 475 | 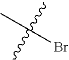 [SMILES: *Br] | 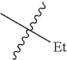 [SMILES: *CC] | 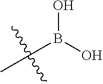 [SMILES: *B(O)O] |
| 476 | 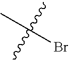 [SMILES: *Br] | 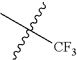 [SMILES: *C(F)(F)F] | 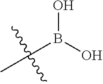 [SMILES: *B(O)O] |
| 477 | 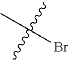 [SMILES: *Br] | 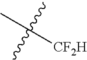 [SMILES: *C(F)F] | 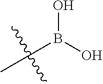 [SMILES: *B(O)O] |
| 478 | 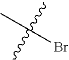 [SMILES: *Br] | 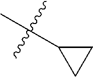 [SMILES: *C1CC1] | 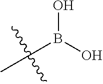 [SMILES: *B(O)O] |
| 479 | 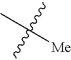 | 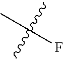 [SMILES: *F] | 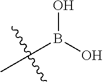 [SMILES: *B(O)O] |
| 480 | 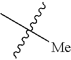 | 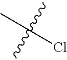 [SMILES: *Cl] | 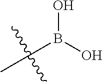 [SMILES: *B(O)O] |
| 481 | 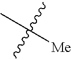 | 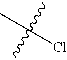 [SMILES: *Cl] | 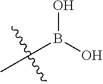 [SMILES: *B(O)O] |
| 482 | 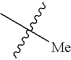 | 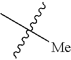 | 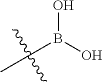 [SMILES: *B(O)O] |
| 483 | 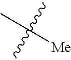 | 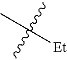 [SMILES: *CC] | 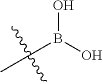 [SMILES: *B(O)O] |
| 484 | 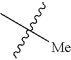 | 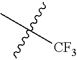 [SMILES: *C(F)(F)F] | 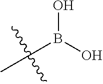 [SMILES: *B(O)O] |
| 485 | 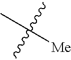 | 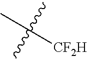 [SMILES: *C(F)F] | 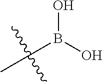 [SMILES: *B(O)O] |
| 486 | 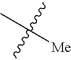 | 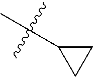 [SMILES: *C1CC1] | 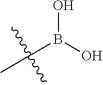 [SMILES: *B(O)O] |
| 487 | 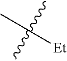 [SMILES: *CC] | 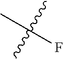 [SMILES: *F] | 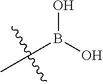 [SMILES: *B(O)O] |
| 488 | 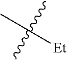 [SMILES: *CC] | 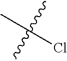 [SMILES: *Cl] | 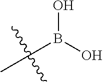 [SMILES: *B(O)O] |
| 489 | 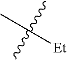 [SMILES: *CC] | 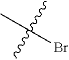 [SMILES: *Br] | 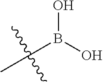 [SMILES: *B(O)O] |
| 490 | 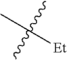 [SMILES: *CC] | 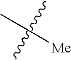 | 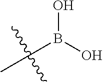 [SMILES: *B(O)O] |
| 491 | 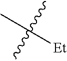 [SMILES: *CC] | 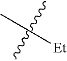 [SMILES: *CC] | 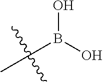 [SMILES: *B(O)O] |
| 492 | 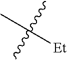 [SMILES: *CC] | 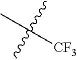 [SMILES: *C(F)(F)F] | 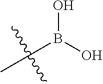 [SMILES: *B(O)O] |
| 493 | 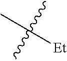 [SMILES: *CC] | 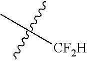 [SMILES: *C(F)F] | 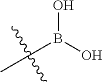 [SMILES: *B(O)O] |
| 494 | 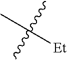 [SMILES: *CC] | 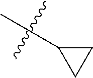 [SMILES: *C1CC1] | 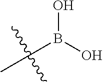 [SMILES: *B(O)O] |
| 495 | 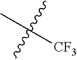 [SMILES: *C(F)(F)F] | 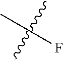 [SMILES: *F] | 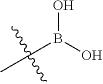 [SMILES: *B(O)O] |
| 496 | 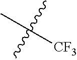 [SMILES: *C(F)(F)F] | 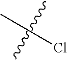 [SMILES: *Cl] | 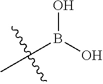 [SMILES: *B(O)O] |
| 497 | 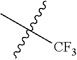 [SMILES: *C(F)(F)F] | 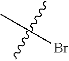 [SMILES: *Br] | 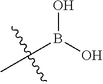 [SMILES: *B(O)O] |
| 498 | 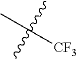 [SMILES: *C(F)(F)F] | 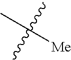 | 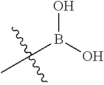 [SMILES: *B(O)O] |
| 499 | 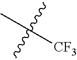 [SMILES: *C(F)(F)F] | 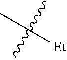 [SMILES: *CC] | 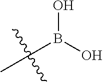 [SMILES: *B(O)O] |
| 500 | 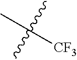 [SMILES: *C(F)(F)F] | 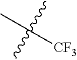 [SMILES: *C(F)(F)F] | 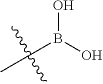 [SMILES: *B(O)O] |
| 501 | 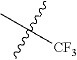 [SMILES: *C(F)(F)F] | 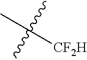 [SMILES: *C(F)F] | 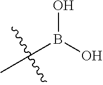 [SMILES: *B(O)O] |
| 502 | 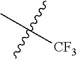 [SMILES: *C(F)(F)F] | 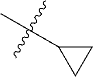 [SMILES: *C1CC1] | 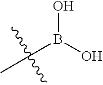 [SMILES: *B(O)O] |
| 503 | 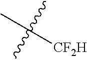 [SMILES: *C(F)F] | 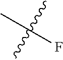 [SMILES: *F] | 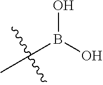 [SMILES: *B(O)O] |
| 504 | 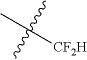 [SMILES: *C(F)F] | 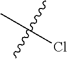 [SMILES: *Cl] | 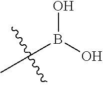 [SMILES: *B(O)O] |
| 505 | 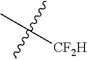 [SMILES: *C(F)F] | 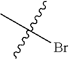 [SMILES: *Br] | 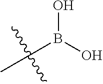 [SMILES: *B(O)O] |
| 506 | 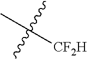 [SMILES: *C(F)F] | 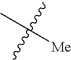 | 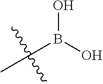 [SMILES: *B(O)O] |
| 507 | 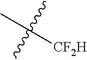 [SMILES: *C(F)F] | 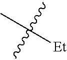 [SMILES: *CC] | 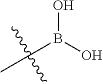 [SMILES: *B(O)O] |
| 508 | 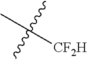 [SMILES: *C(F)F] | 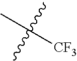 [SMILES: *C(F)(F)F] | 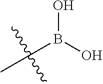 [SMILES: *B(O)O] |
| 509 | 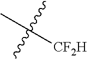 [SMILES: *C(F)F] | 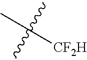 [SMILES: *C(F)F] | 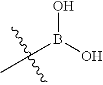 [SMILES: *B(O)O] |
| 510 | 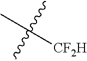 [SMILES: *C(F)F] | 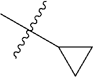 [SMILES: *C1CC1] | 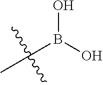 [SMILES: *B(O)O] |
| 511 | 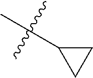 [SMILES: *C1CC1] | 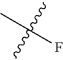 [SMILES: *F] | 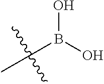 [SMILES: *B(O)O] |
| 512 | 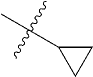 [SMILES: *C1CC1] | 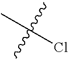 [SMILES: *Cl] | 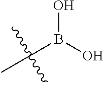 [SMILES: *B(O)O] |
| 513 | 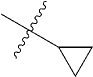 [SMILES: *C1CC1] | 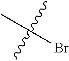 [SMILES: *Br] | 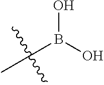 [SMILES: *B(O)O] |
| 514 | 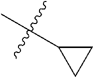 [SMILES: *C1CC1] | 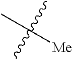 | 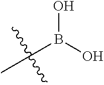 [SMILES: *B(O)O] |
| 515 | 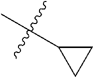 [SMILES: *C1CC1] | 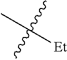 [SMILES: *CC] | 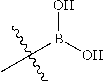 [SMILES: *B(O)O] |
| 516 | 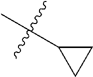 [SMILES: *C1CC1] | 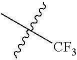 [SMILES: *C(F)(F)F] | 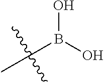 [SMILES: *B(O)O] |
| 517 | 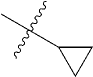 [SMILES: *C1CC1] | 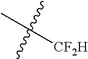 [SMILES: *C(F)F] | 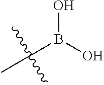 [SMILES: *B(O)O] |
| 518 | 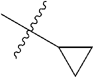 [SMILES: *C1CC1] | 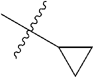 [SMILES: *C1CC1] | 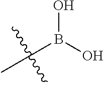 [SMILES: *B(O)O] |

In certain embodiments, the present invention provides compounds represented by Formula (XVIII) or (XIX), or a pharmaceutically acceptable salt or ester thereof:

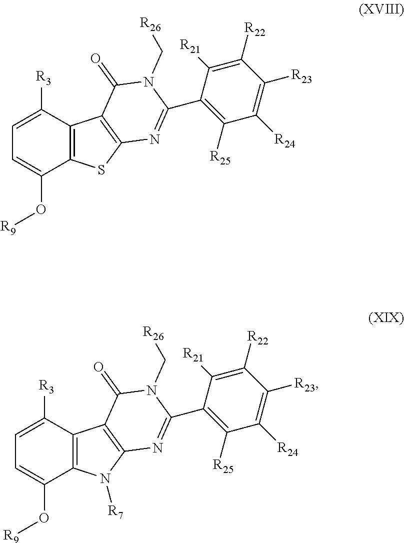

wherein each R₂₁, R₂₂, R₂₃, R₂₄, or R₂₅ is independently hydrogen, halogen, optionally substituted —C₁-C₆ alkyl, optionally substituted —C₁-C₆ alkoxyl, or optionally substituted —C₃-C₈-cycloalkyl; R₂₆ is optionally substituted aryl, optionally substituted heteroaryl, optionally substituted heteroarylalkyl, optionally substituted —C₃-C₈-cycloalkyl, optionally substituted —C₁-C₆ alkyl, —NR₁₁R₁₂, —CH₂NR₁₁R₁₂, —CH₂NR₁₁C(O)R₁₂,

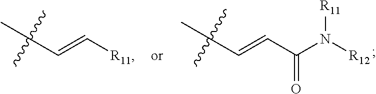

and R₃, R₁₁, R₁₂, and R₉ are as previously defined. Preferably, R₂₆ is optionally substituted aryl or optionally substituted heteroaryl.

Alternatively, R₂₁ and R₂₂ are taken together with the carbon atoms to which they are attached to form an optionally substituted carbocyclic or heterocyclic ring which is fused with phenyl.

Alternatively, R₂₂ and R₂₃ are taken together with the carbon atoms to which they are attached to form an optionally substituted carbocyclic or heterocyclic ring which is fused with phenyl.

Alternatively, R₇ and R₈ are taken together with the nitrogen atom to which they are attached to form an optionally substituted heterocyclic ring.

In certain embodiments, the present invention provides compounds represented by Formula (XX) or (XXI), or a pharmaceutically acceptable salt or ester thereof:

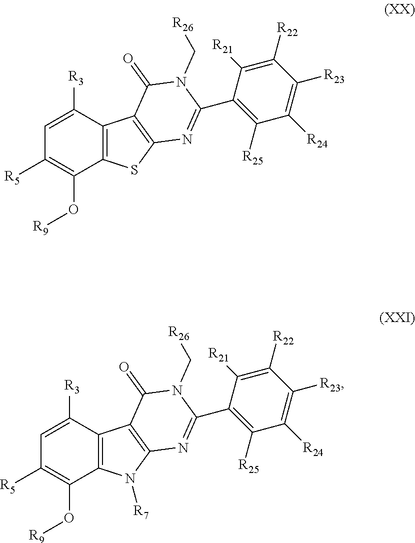

wherein R₂₁, R₂₂, R₂₃, R₂₄, R₂₅, R₂₆, R₃, R₅, R₇, and R₉ are as previously defined, and R₅ is not hydrogen. Preferably, R₂₆ is optionally substituted aryl or optionally substituted heteroaryl.

Alternatively, R₂₁ and R₂₂ are taken together with the carbon atoms to which they are attached to form an optionally substituted carbocyclic or heterocyclic ring which is fused with phenyl.

Alternatively, R₂₂ and R₂₃ are taken together with the carbon atoms to which they are attached to form an optionally substituted carbocyclic or heterocyclic ring which is fused with phenyl.

In certain embodiments, the present invention provides compounds represented by Formula (XXII) or (XXIII), or a pharmaceutically acceptable salt or ester thereof:

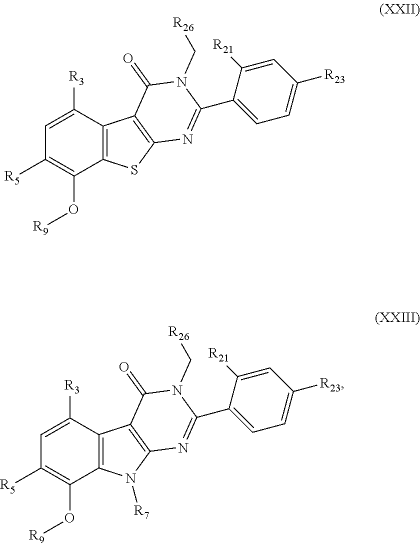

wherein R₂₁, R₂₃, R₃, R₅, R₇, and R₉ are as previously defined. Preferably, R₅ is not hydrogen, and R₂₆ is optionally substituted aryl or optionally substituted heteroaryl.

In certain embodiments, the present invention provides compounds represented by Formula (XXIV), or a pharmaceutically acceptable salt or ester thereof:

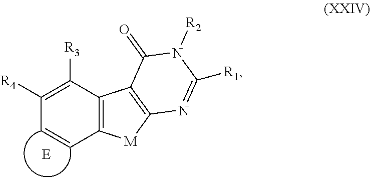

wherein E is optionally substituted 4 to 8 membered heterocycloalkyl or optionally substituted heteroaryl; M, R₁, R₂, R₃, and R₄ are as previously defined. Preferably, E is optionally substituted 5 to 6 membered heterocycloalkyl or optionally substituted 5 to 6 membered heteroaryl.

In certain embodiments, the present invention provides compounds represented by Formula (XXV), or (XXVI), a pharmaceutically acceptable salt or ester thereof:

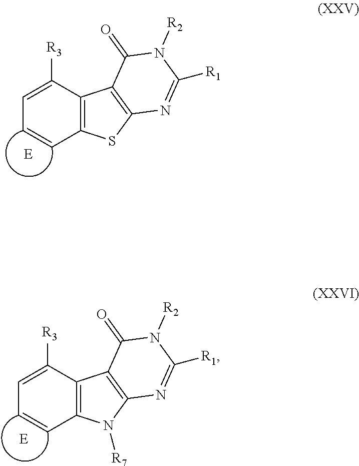

wherein E, R₁, R₂, R₃, and R₇ are as previously defined.

In certain embodiments, the present invention provides compounds represented by Formula (XXVII) or (XXVIII), or a pharmaceutically acceptable salt or ester thereof:

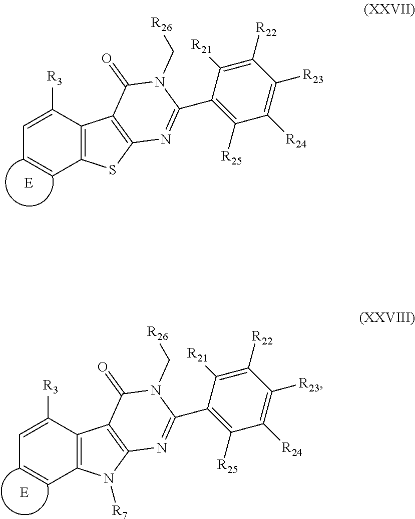

wherein E, R₂₁, R₂₂, R₂₃, R₂₄, R₂₅, R₂₆, R₃, and R₇ are as previously defined.

In certain embodiments, the present invention provides compounds represented by Formula (XXIX) or (XXX), or a pharmaceutically acceptable salt or ester thereof:

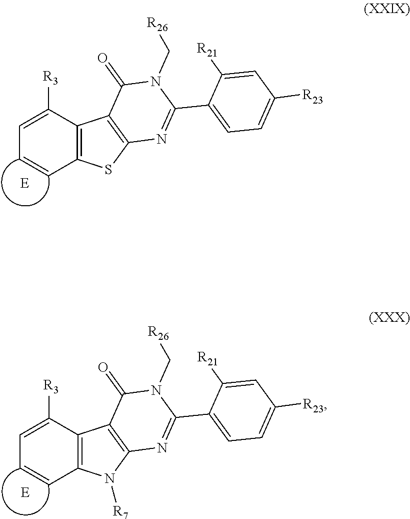

wherein E, R₂₁, R₂₃, R₂₆, R₃, and R₇ are as previously defined.

In certain embodiments, the present invention provides compounds represented by one of Formulae (XXXI-1)˜(XXX1-6), or a pharmaceutically acceptable salt or ester thereof:

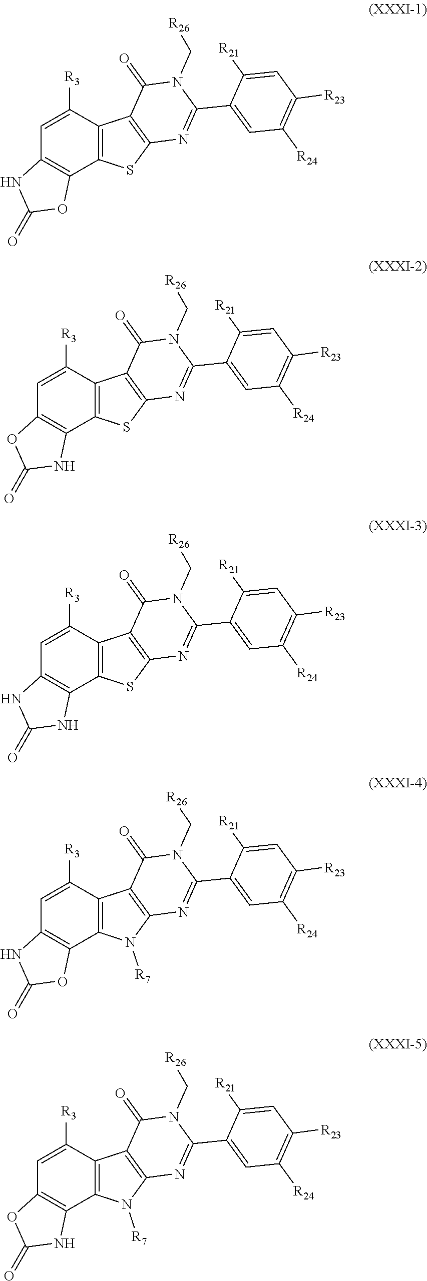

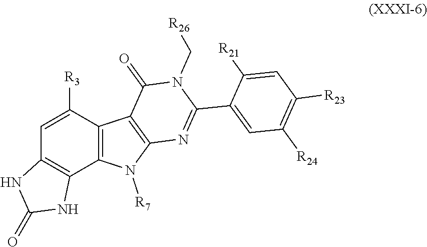

wherein R₂₁, R₂₃, R₂₄, R₂₆, R₃, and R₇ are as previously defined.

In certain embodiments, the present invention provides compounds represented by one of Formulae (XXXII-1)˜(XXXII-3), or a pharmaceutically acceptable salt or ester thereof:

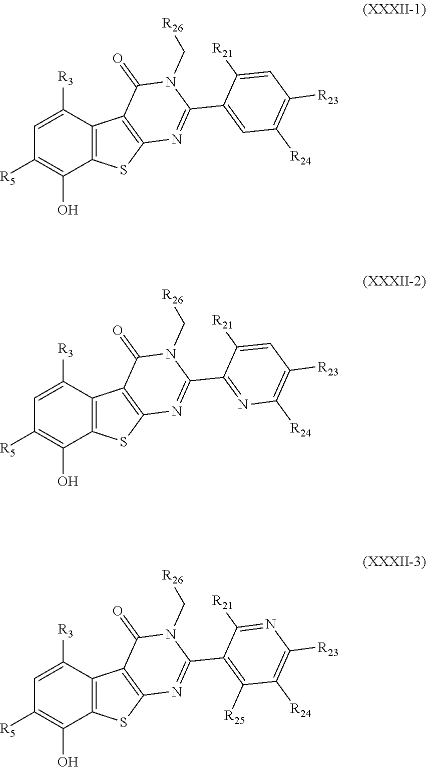

wherein R₂₁, R₂₃, R₂₄, R₂₅, R₂₆, R₃, and R₅ are as previously defined. Preferably, R₃ is hydrogen or halogen, R₅ is halogen, and R₂₆ is optionally substituted aryl or optionally substituted heteroaryl.

In certain embodiments, the present invention provides compounds represented by one of Formulae (XXXIII-1)˜(XXXIII-3), or a pharmaceutically acceptable salt or ester thereof:

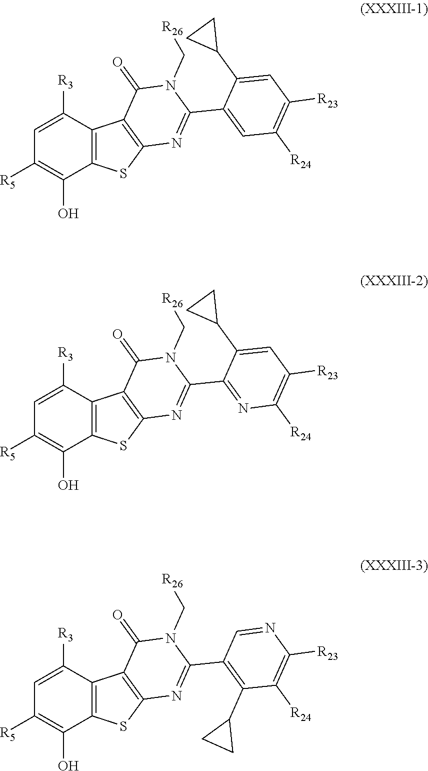

wherein R₂₃, R₂₄, R₂₆, R₃, and R₅ are as previously defined. Preferably, R₃ is hydrogen or halogen, R₅ is halogen, and R₂₆ is optionally substituted aryl or optionally substituted heteroaryl.

In certain embodiments, the present invention provides compounds represented by one of Formulae (XXXIV-1)˜(XXXIV-3), or a pharmaceutically acceptable salt or ester thereof:

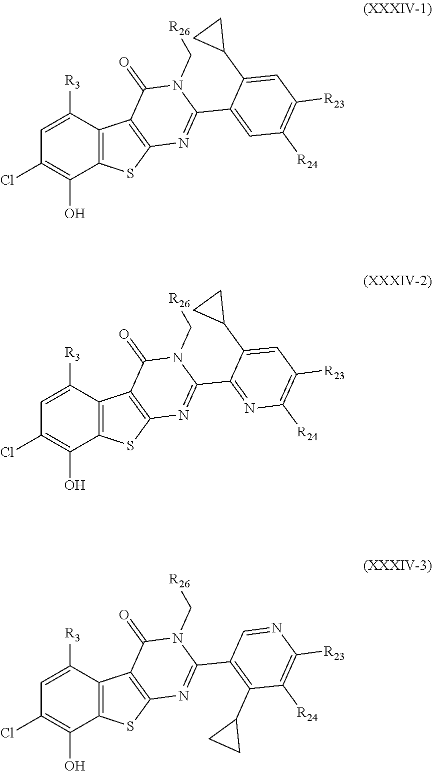

wherein R₂₃, R₂₄, R₂₆, and R₃ are as previously defined. Preferably, R₃ is hydrogen or halogen, and R₂₆ is optionally substituted aryl or optionally substituted heteroaryl.

In certain embodiments, the present invention provides compounds represented by one of Formulae (XXXV-1)˜(XXXV-3), or a pharmaceutically acceptable salt or ester thereof:

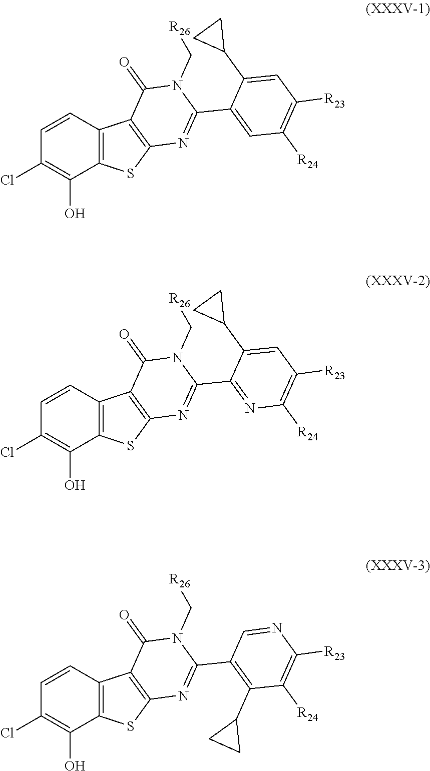

wherein R₂₃, R₂₄, and R₂₆ are as previously defined. Preferably R₂₆ is optionally substituted aryl or optionally substituted heteroaryl.

In certain embodiments, the present invention provides compounds represented by one of Formulae (XXXVI-1)˜(XXXVI-10), or a pharmaceutically acceptable salt or ester thereof:

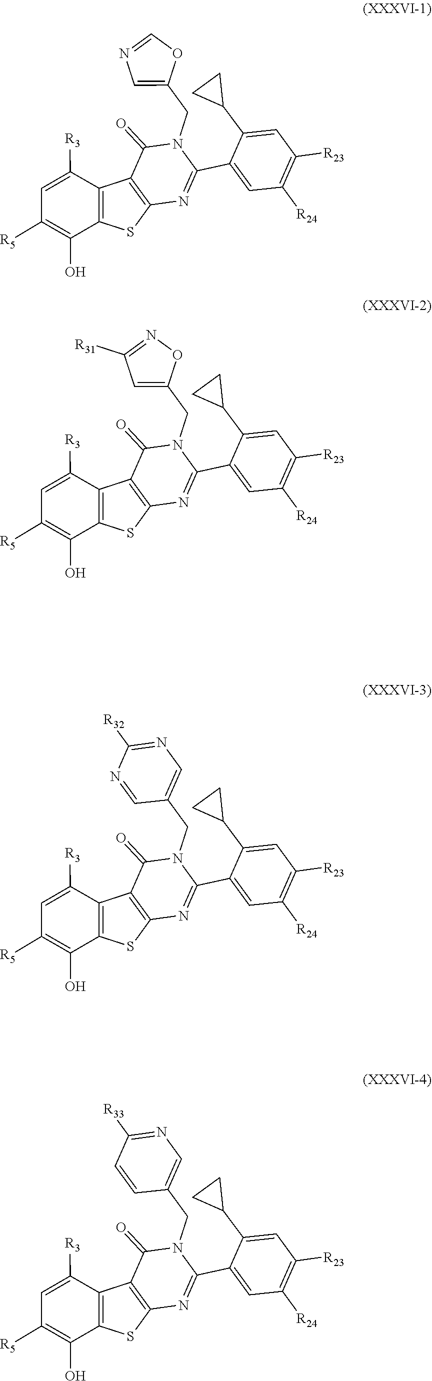

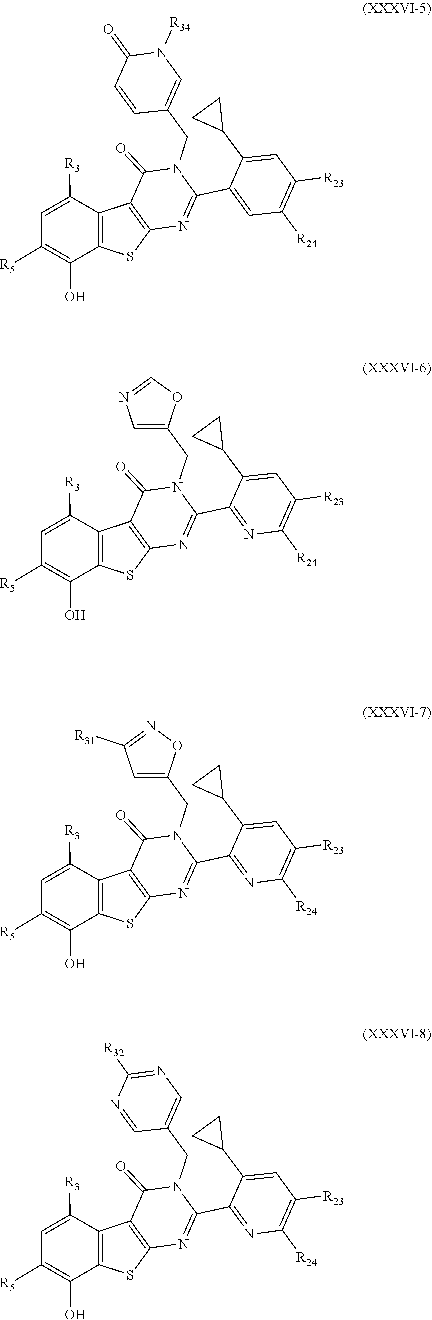

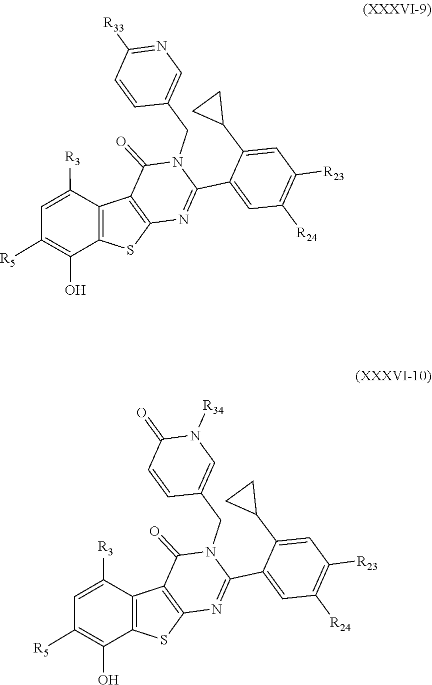

wherein each R₃₁, R₃₂, R₃₃, or R₃₄ is independently hydrogen, halogen, optionally substituted —C₁-C₆ alkyl, optionally substituted —C₁-C₆ alkoxyl, or optionally substituted —C₃-C₈-cycloalkyl; R₂₃, R₂₄, R₂₆, R₅ and R₃ are as previously defined. Preferably R₅ is not hydrogen.

In certain embodiments, the present invention provides compounds represented by one of Formulae (XXXVII-1)˜(XXXVII-10), or a pharmaceutically acceptable salt or ester thereof:

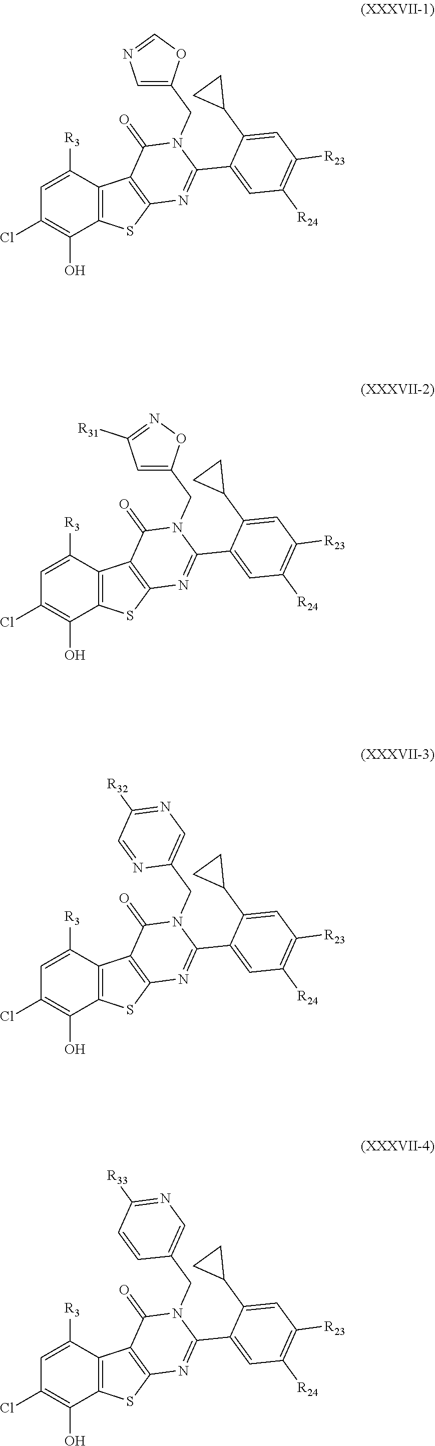

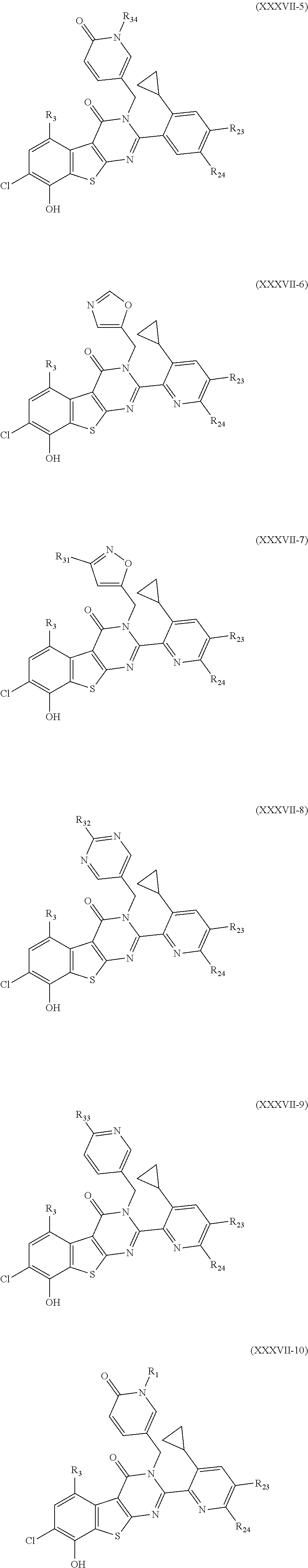

wherein R₂₃, R₂₄, R₃₁, R₃₂, R₃₃, R₃₄, and R₃ are as previously defined. Preferably R₂₃ is —F, —CF₃, —OCH₃, or —S(O)₂CH₃, R₃₁ is —CF₃, R₃₂ is —CF₃, R₃₃ is —CF₃, and R₃₄ is hydrogen or —CH₃.

In certain embodiments, the present invention provides compounds represented by one of Formulae (XXXVIII-1)˜(XXXVIII-10), or a pharmaceutically acceptable salt or ester thereof:

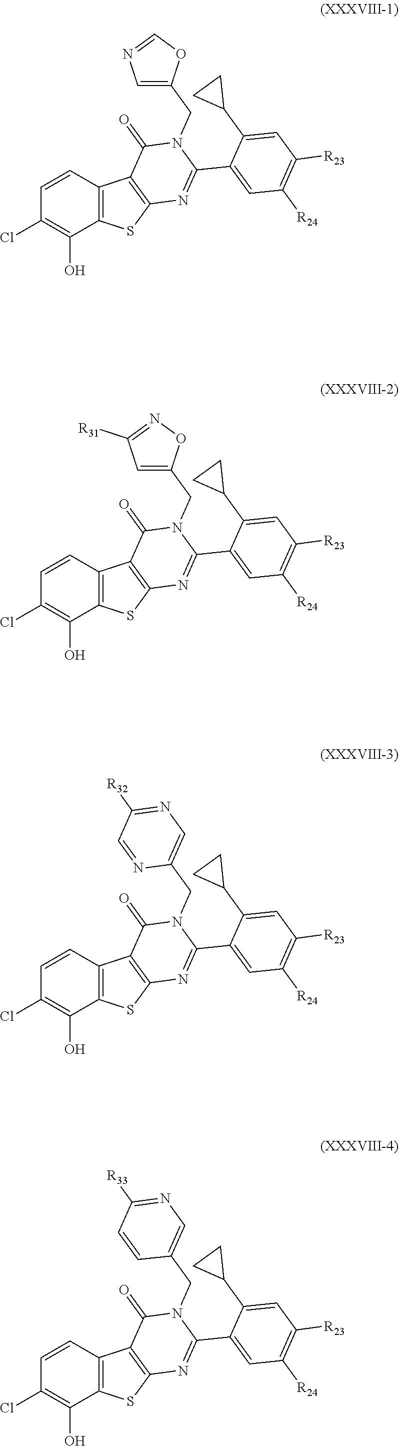

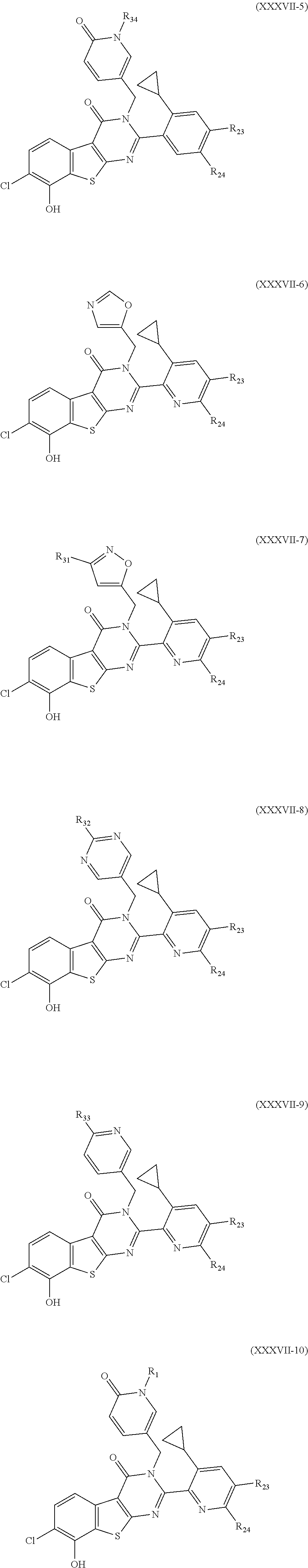

wherein R₂₃, R₂₄, R₃₁, R₃₂, R₃₃, and R₃₄ are as previously defined. Preferably R₂₃ is —F, —CF₃, —OCH₃, or —S(O)₂CH₃, R₃₁ is —CF₃, R₃₂ is —CF₃, R₃₃ is —CF₃, and R₃₄ is hydrogen or —CH₃.

In certain embodiments, the present invention provides a pharmaceutical composition comprising a therapeutically effective amount of a compound or combination of compounds of the present invention, or a pharmaceutically acceptable salt, ester or combination thereof, in combination with a pharmaceutically acceptable carrier or excipient.

In certain embodiments, the present invention provides a method for the prevention or treatment of an 17β-HSD13 mediated disease or condition. The method comprises administering a therapeutically effective amount of a compound of Formula (I) or a pharmaceutically acceptable salt thereof. The present invention also provides the use of a compound of Formula (I), or a pharmaceutically acceptable salt thereof, in the manufacture of a medicament for the prevention or treatment of a 17β-HSD13 mediated disease or condition including, but not limited to: nonalcoholic fatty liver disease (NAFLD), nonalcoholic steatohepatitis (NASH), liver cirrhosis, liver fibrosis, hepatocellular carcinoma (HCC), and metabolic disorders.

It will be appreciated that the description of the present invention herein should be construed in congruity with the laws and principles of chemical bonding. In some instances, it may be necessary to remove a hydrogen atom to accommodate a substituent at any given location.

It will yet be appreciated that the compounds of the present invention may contain one or more asymmetric carbon atoms and may exist in racemic, diastereoisomeric, and optically active forms. It will still be appreciated that certain compounds of the present invention may exist in different tautomeric forms. All tautomers are contemplated to be within the scope of the present invention.

It should be understood that the compounds encompassed by the present invention are those that are suitably stable for use as pharmaceutical agents.

### Definitions

Listed below are definitions of various terms used to describe this invention. These definitions apply to the terms as they are used throughout this specification and claims, unless otherwise limited in specific instances, either individually or as part of a larger group.

The term “aryl,” as used herein, refers to a mono- or polycyclic carbocyclic ring system comprising at least one aromatic ring. Preferred aryl groups are C₆-C₁₂-aryl groups, including, but not limited to, phenyl, naphthyl, tetrahydronaphthyl, indanyl, and indenyl. A polycyclic aryl is a polycyclic ring system that comprises at least one aromatic ring. Polycyclic aryls can comprise fused rings, covalently attached rings or a combination thereof.

The term “heteroaryl,” as used herein, refers to a mono- or polycyclic aromatic radical having one or more ring atom selected from S, O and N; and the remaining ring atoms are carbon, wherein any N or S contained within the ring may be optionally oxidized. In certain embodiments, a heteroaryl group is a 5- to 10-membered heteroaryl, such as a 5- or 6-membered monocyclic heteroaryl or an 8- to 10-membered bicyclic heteroaryl. Heteroaryl groups include, but are not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzoxazolyl, quinoxalinyl. A polycyclic heteroaryl can comprise fused rings, covalently attached rings or a combination thereof. A heteroaryl group can be C-attached or N-attached where possible.

In accordance with the invention, aryl and heteroaryl groups can be substituted or unsubstituted.

The term “bicyclic aryl” or “bicyclic heteroaryl” refers to a ring system consisting of two rings wherein at least one ring is aromatic; and the two rings can be fused or covalently attached.

The term “alkyl” as used herein, refers to saturated, straight- or branched-chain hydrocarbon radicals. “C₁-C₄ alkyl,” “C₁-C₆ alkyl,” “C₁-C₈ alkyl,” “C₁-C₁₂ alkyl,” “C₂-C₄ alkyl,” and “C₃-C₆ alkyl,” refer to alkyl groups containing from 1 to 4, 1 to 6, 1 to 8, 1 to 12, 2 to 4 and 3 to 6 carbon atoms respectively. Examples of alkyl groups include, but are not limited to, methyl, ethyl, propyl, isopropyl, n-butyl, sec-butyl, tert-butyl, n-pentyl, neopentyl, n-hexyl, n-heptyl and n-octyl radicals.

The term “alkenyl” as used herein, refers to straight- or branched-chain hydrocarbon radicals having at least one carbon-carbon double bond. “C₂-C₈ alkenyl,” “C₂-C₁₂ alkenyl,” “C₂-C₄ alkenyl,” “C₃-C₄ alkenyl,” and “C₃-C₆ alkenyl,” refer to alkenyl groups containing from 2 to 8, 2 to 12, 2 to 4, 3 to 4 or 3 to 6 carbon atoms respectively. Alkenyl groups include, but are not limited to, ethenyl, propenyl, butenyl, 2-methyl-2-buten-2-yl, heptenyl, octenyl, and the like.

The term “alkynyl” as used herein, refers to straight- or branched-chain hydrocarbon radicals having at least one carbon-carbon triple bond. “C₂-C₈ alkynyl,” “C₂-C₁₂ alkynyl,” “C₂-C₄ alkynyl,” “C₃-C₄ alkynyl,” and “C₃-C₆ alkynyl,” refer to alkynyl groups containing from 2 to 8, 2 to 12, 2 to 4, 3 to 4 or 3 to 6 carbon atoms respectively. Representative alkynyl groups include, but are not limited to, ethynyl, 2-propynyl, 2-butynyl, heptynyl, octynyl, and the like.

The term “cycloalkyl”, as used herein, refers to a monocyclic or polycyclic saturated carbocyclic ring, such as a bi- or tri-cyclic fused, bridged or spiro system. The ring carbon atoms are optionally oxo-substituted or optionally substituted with an exocyclic olefinic double bond. Preferred cycloalkyl groups include C₃-C₁₂ cycloalkyl, C₃-C₆ cycloalkyl, C₃-C₈ cycloalkyl and C₄-C₇ cycloalkyl. Examples of cycloalkyl include, but are not limited to, cyclopropyl, cyclobutyl, cyclopentyl, cyclohexyl, cyclopentyl, cyclooctyl, 4-methylene-cyclohexyl, bicyclo[2.2.1]heptyl, bicyclo[3.1.0]hexyl, spiro[2.5]octyl, 3-methylenebicyclo[3.2.1]octyl, spiro[4.4]nonanyl, and the like.

The term “cycloalkenyl”, as used herein, refers to monocyclic or polycyclic carbocyclic ring, such as a bi- or tri-cyclic fused, bridged or spiro system having at least one carbon-carbon double bond. The ring carbon atoms are optionally oxo-substituted or optionally substituted with an exocyclic olefinic double bond. Preferred cycloalkenyl groups include C₃-C₁₂ cycloalkenyl, C₄-C₁₂-cycloalkenyl, C₃-C₈ cycloalkenyl, C₄-C₈ cycloalkenyl and C₅-C₇ cycloalkenyl groups. Examples of cycloalkenyl include, but are not limited to, cyclopropenyl, cyclobutenyl, cyclopentenyl, cyclohexenyl, cycloheptenyl, cyclooctenyl, bicyclo[2.2.1]hept-2-enyl, bicyclo[3.1.0]hex-2-enyl, spiro[2.5]oct-4-enyl, spiro[4.4]non-2-enyl, bicyclo[4.2.1]non-3-en-12-yl, and the like.

As used herein, the term “arylalkyl” means a functional group wherein an alkylene chain is attached to an aryl group, e.g., —(CH₂)ₙ-phenyl, where n is 1 to 12, preferably 1 to 6 and more preferably 1 or 2. The term “substituted arylalkyl” means an arylalkyl functional group in which the aryl group is substituted. Similarly, the term “heteroarylalkyl” means a functional group wherein an alkylene chain, is attached to a heteroaryl group, e.g., —(CH₂)ₙ-heteroaryl, where n is 1 to 12, preferably 1 to 6 and more preferably 1 or 2. The term “substituted heteroarylalkyl” means a heteroarylalkyl functional group in which the heteroaryl group is substituted.

As used herein, the term “alkoxy” refers to a radical in which an alkyl group having the designated number of carbon atoms is connected to the rest of the molecule via an oxygen atom. Alkoxy groups include C₁-C₁₂-alkoxy, C₁-C₈-alkoxy, C₁-C₆-alkoxy, C₁-C₄-alkoxy and C₁-C₃-alkoxy groups. Examples of alkoxy groups include, but are not limited to, methoxy, ethoxy, n-propoxy, 2-propoxy (isopropoxy) and the higher homologs and isomers. Preferred alkoxy is C₁-C₃ alkoxy.

An “aliphatic” group is a non-aromatic moiety comprised of any combination of carbon atoms, hydrogen atoms, halogen atoms, oxygen, nitrogen or other atoms, and optionally contains one or more units of unsaturation, e.g., double and/or triple bonds. Examples of aliphatic groups are functional groups, such as alkyl, alkenyl, alkynyl, O, OH, NH, NH₂, C(O), S(O)₂, C(O)O, C(O)NH, OC(O)O, OC(O)NH, OC(O)NH₂, S(O)₂NH, S(O)₂NH₂, NHC(O)NH₂, NHC(O)C(O)NH, NHS(O)₂NH, NHS(O)₂NH₂, C(O)NHS(O)₂, C(O)NHS(O)₂NH or C(O)NHS(O)₂NH₂, and the like, groups comprising one or more functional groups, non-aromatic hydrocarbons (optionally substituted), and groups wherein one or more carbons of a non-aromatic hydrocarbon (optionally substituted) is replaced by a functional group. Carbon atoms of an aliphatic group can be optionally oxo-substituted. An aliphatic group may be straight chained, branched, cyclic, or a combination thereof and preferably contains between about 1 and about 24 carbon atoms, more typically between about 1 and about 12 carbon atoms. In addition to aliphatic hydrocarbon groups, as used herein, aliphatic groups expressly include, for example, alkoxyalkyls, polyalkoxyalkyls, such as polyalkylene glycols, polyamines, and polyimines.

Aliphatic groups may be optionally substituted.

The terms “heterocyclic” and “heterocycloalkyl” can be used interchangeably and refer to a non-aromatic ring or a polycyclic ring system, such as a bi- or tri-cyclic fused, bridged or spiro system, where (i) each ring system contains at least one heteroatom independently selected from oxygen, sulfur and nitrogen, (ii) each ring system can be saturated or unsaturated (iii) the nitrogen and sulfur heteroatoms may optionally be oxidized, (iv) the nitrogen heteroatom may optionally be quaternized, (v) any of the above rings may be fused to an aromatic ring, and (vi) the remaining ring atoms are carbon atoms which may be optionally oxo-substituted or optionally substituted with exocyclic olefinic double bond. Representative heterocycloalkyl groups include, but are not limited to, 1,3-dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, 2-azabicyclo[2.2.1]-heptyl, 8-azabicyclo[3.2.1]octyl, 5-azaspiro[2.5]octyl, 2-oxa-7-azaspiro[4.4]nonanyl, 7-oxooxepan-4-yl, and tetrahydrofuryl. Such heterocyclic or heterocycloalkyl groups may be further substituted. A heterocycloalkyl or heterocyclic group can be C-attached or N-attached where possible.

It is understood that any alkyl, alkenyl, alkynyl, alicyclic, cycloalkyl, cycloalkenyl, aryl, heteroaryl, heterocyclic, aliphatic moiety or the like described herein can also be a divalent or multivalent group when used as a linkage to connect two or more groups or substituents, which can be at the same or different atom(s). One of skill in the art can readily determine the valence of any such group from the context in which it occurs.

The term “substituted” refers to substitution by independent replacement of one, two, or three or more of the hydrogen atoms with substituents including, but not limited to, —F, —Cl, —Br, —I, —OH, C₁-C₁₂-alkyl; C₂-C₁₂-alkenyl, C₂-C₁₂-alkynyl, —C₃-C₁₂-cycloalkyl, protected hydroxy, —NO₂, —N₃, —CN, —NH₂, protected amino, oxo, thioxo, —NH—C₁-C₁₂-alkyl, —NH—C₂-C₈-alkenyl, —NH—C₂-C₈-alkynyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₈-alkenyl, —O—C₂-C₈-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₈-alkenyl, —C(O)—C₂-C₈-alkynyl, —C(O)—C₃-C₁₂-cycloalkyl, —C(O)-aryl, —C(O)— heteroaryl, —C(O)-heterocycloalkyl, —CONH₂, —CONH—C₁-C₁₂-alkyl, —CONH—C₂-C₈-alkenyl, —CONH—C₂-C₈-alkynyl, —CONH—C₃-C₁₂-cycloalkyl, —CONH-aryl, —CONH-heteroaryl, —CONH-heterocycloalkyl, —OCO₂—C₁-C₁₂-alkyl, —OCO₂—C₂-C₈-alkenyl, —OCO₂—C₂-C₈-alkynyl, —OCO₂—C₃-C₁₂-cycloalkyl, —OCO₂-aryl, —OCO₂-heteroaryl, —OCO₂-heterocycloalkyl, —CO₂—C₁-C₁₂ alkyl, —CO₂—C₂-C₈ alkenyl, —CO₂—C₂-C₈ alkynyl, —CO₂—C₃-C₁₂-cycloalkyl, —CO₂-aryl, —CO₂-heteroaryl, —CO₂-heterocyloalkyl, —OCONH₂, —OCONH—C₁-C₁₂-alkyl, —OCONH—C₂-C₈-alkenyl, —OCONH—C₂-C₈-alkynyl, —OCONH—C₃-C₁₂-cycloalkyl, —OCONH-aryl, —OCONH-heteroaryl, —OCONH-heterocycloalkyl, —NHC(O)H, —NHC(O)—C₁-C₁₂-alkyl, —NHC(O)—C₂-C₈-alkenyl, —NHC(O)—C₂-C₈-alkynyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₈-alkenyl, —NHCO₂—C₂-C₈-alkynyl, —NHCO₂—C₃-C₁₂-cycloalkyl, —NHCO₂-aryl, —NHCO₂-heteroaryl, —NHCO₂-heterocycloalkyl, —NHC(O)NH₂, —NHC(O)NH—C₁-C₁₂-alkyl, —NHC(O)NH—C₂-C₈-alkenyl, —NHC(O)NH—C₂-C₈-alkynyl, —NHC(O)NH—C₃-C₁₂-cycloalkyl, —NHC(O)NH-aryl, —NHC(O)NH-heteroaryl, —NHC(O)NH-heterocycloalkyl, —NHC(S)NH₂, —NHC(S)NH—C₁-C₁₂-alkyl, —NHC(S)NH—C₂-C₈-alkenyl, —NHC(S)NH—C₂-C₈-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₈-alkenyl, —NHC(NH)NH—C₂-C₈-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₈-alkenyl, —NHC(NH)—C₂-C₈-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH₂, —C(NH)NH—C₁-C₁₂-alkyl, —C(NH)NH—C₂-C₈-alkenyl, —C(NH)NH—C₂-C₈-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₈-alkenyl, —S(O)—C₂-C₈-alkynyl, —S(O)—C₃-C₁₂-cycloalkyl, —S(O)-aryl, —S(O)-heteroaryl, —S(O)-heterocycloalkyl, —SO₂NH₂, —SO₂NH—C₁-C₁₂-alkyl, —SO₂NH—C₂-C₈-alkenyl, —SO₂NH—C₂-C₈-alkynyl, —SO₂—C₁-C₁₂-alkyl, —SO₂—C₂-C₈-alkenyl, —SO₂—C₂-C₈-alkynyl, —SO₂—C₃-C₁₂-cycloalkyl, —SO₂-aryl, —SO₂-heteroaryl, —SO₂-heterocycloalkyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₈-alkenyl, —NHSO₂—C₂-C₈-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₈-alkenyl, —S—C₂-C₈-alkynyl, —S—C₃-C₁₂-cycloalkyl, —S-aryl, —S-heteroaryl, —S-heterocycloalkyl, or methylthio-methyl. In certain embodiments, the substituents are independently selected from halo, preferably Cl and F; C₁-C₄-alkyl, preferably methyl and ethyl; halo-C₁-C₄-alkyl, such as fluoromethyl, difluoromethyl, and trifluoromethyl; C₂-C₄-alkenyl; halo-C₂-C₄-alkenyl; C₃-C₆-cycloalkyl, such as cyclopropyl; C₁-C₄-alkoxy, such as methoxy and ethoxy; halo-C₁-C₄-alkoxy, such as fluoromethoxy, difluoromethoxy, and trifluoromethoxy; —CN; —OH; NH₂; C₁-C₄-alkylamino; di(C₁-C₄-alkyl)amino; and NO₂. It is understood that an aryl, heteroaryl, alkyl, alkenyl, alkynyl, cycloalkyl, or heterocycloalkyl in a substituent can be further substituted. In certain embodiments, a substituent in a substituted moiety is additionally optionally substituted with one or more groups, each group being independently selected from C₁-C₄-alkyl; —CF₃, —OCH₃, —OCF₃, —F, —Cl, —Br, —I, —OH, —NO₂, —CN, and —NH₂. Preferably, a substituted alkyl group is substituted with one or more halogen atoms, more preferably, one or more fluorine or chlorine atoms.

The term “halo” or halogen” alone or as part of another substituent, as used herein, refers to a fluorine, chlorine, bromine, or iodine atom.

The term “optionally substituted”, as used herein, means that the referenced group may be substituted or unsubstituted. In one embodiment, the referenced group is optionally substituted with zero substituents, i.e., the referenced group is unsubstituted. In certain embodiments, the referenced group is optionally substituted with one or more additional group(s) individually and independently selected from groups described herein.

The term “hydrogen” includes hydrogen and deuterium. In addition, the recitation of an element includes all isotopes of that element so long as the resulting compound is pharmaceutically acceptable. In certain embodiments, the isotopes of an element are present at a particular position according to their natural abundance. In other embodiments, one or more isotopes of an element at a particular position are enriched beyond their natural abundance.

The term “hydroxy activating group,” as used herein, refers to a labile chemical moiety which is known in the art to activate a hydroxyl group so that it will depart during synthetic procedures such as in a substitution or an elimination reaction. Examples of hydroxyl activating group include, but not limited to, mesylate, tosylate, triflate, p-nitrobenzoate, phosphonate and the like.

The term “activated hydroxyl,” as used herein, refers to a hydroxy group activated with a hydroxyl activating group, as defined above, including, but not limited to mesylate, tosylate, triflate, p-nitrobenzoate, phosphonate groups.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the art are described generally in P. G. M. Wuts, Greene's Protective Groups in Organic Synthesis, 5th edition, John Wiley & Sons, Hoboken, NJ (2014). Examples of hydroxyl protecting groups include, but are not limited to, benzyloxycarbonyl, 4-methoxybenzyloxycarbonyl, tert-butoxycarbonyl, isopropoxycarbonyl, diphenylmethoxycarbonyl, 2,2,2-trichloroethoxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, allyl, benzyl, triphenyl-methyl (trityl), methoxymethyl, methylthiomethyl, benzyloxymethyl, 2-(trimethylsilyl)-ethoxymethyl, methanesulfonyl, trimethylsilyl, triisopropylsilyl, and the like.

The term “protected hydroxy,” as used herein, refers to a hydroxy group protected with a hydroxy protecting group, as defined above, including but not limited to, benzoyl, acetyl, trimethylsilyl, triethylsilyl, methoxymethyl groups, for example.

The term “hydroxy prodrug group,” as used herein, refers to a promoiety group which is known in the art to change the physicochemical, and hence the biological properties of a parent drug in a transient manner by covering or masking the hydroxy group. After said synthetic procedure(s), the hydroxy prodrug group as described herein must be capable of reverting back to hydroxy group in vivo. Hydroxy prodrug groups as known in the art are described generally in Kenneth B. Sloan, Prodrugs, Topical and Ocular Drug Delivery, (Drugs and the Pharmaceutical Sciences; Volume 53), Marcel Dekker, Inc., New York (1992).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the art are described generally in P. G. M. Wuts, Greene's Protective Groups in Organic Synthesis, 5th edition, John Wiley & Sons, Hoboken, NJ (2014). Examples of amino protecting groups include, but are not limited to, methoxycarbonyl, t-butoxycarbonyl, 12-fluorenyl-methoxycarbonyl, benzyloxycarbonyl, and the like.

The term “protected amino,” as used herein, refers to an amino group protected with an amino protecting group as defined above.

The term “leaving group” means a functional group or atom which can be displaced by another functional group or atom in a substitution reaction, such as a nucleophilic substitution reaction. By way of example, representative leaving groups include chloro, bromo and iodo groups; sulfonic ester groups, such as mesylate, tosylate, brosylate, nosylate and the like; and acyloxy groups, such as acetoxy, trifluoroacetoxy and the like.

The term “aprotic solvent,” as used herein, refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heterocyclic compounds, such as, for example, tetrahydrofuran and N-methylpyrrolidinone, and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

The term “protic solvent,” as used herein, refers to a solvent that tends to provide protons, such as an alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

Combinations of substituents and variables envisioned by this invention are only those that result in the formation of stable compounds. The term “stable,” as used herein, refers to compounds which possess stability sufficient to allow manufacture and which maintains the integrity of the compound for a sufficient period of time to be useful for the purposes detailed herein (e.g., therapeutic or prophylactic administration to a subject).

The synthesized compounds can be separated from a reaction mixture and further purified by a method such as column chromatography, high pressure liquid chromatography, or recrystallization. As can be appreciated by the skilled artisan, further methods of synthesizing the compounds of the Formula herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2^{nd} Ed. Wiley-VCH (1999); P. G. M. Wuts, Greene's Protective Groups in Organic Synthesis, 5th edition, John Wiley & Sons, Hoboken, NJ (2014); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The term “subject,” as used herein, refers to an animal. Preferably, the animal is a mammal. More preferably, the mammal is a human. A subject also refers to, for example, a dog, cat, horse, cow, pig, guinea pig, fish, bird and the like.

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. Tautomers may be in cyclic or acyclic. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

Certain compounds of the present invention may also exist in different stable conformational forms which may be separable. Torsional asymmetry due to restricted rotation about an asymmetric single bond, for example because of steric hindrance or ring strain, may permit separation of different conformers. The present invention includes each conformational isomer of these compounds and mixtures thereof.

As used herein, the term “pharmaceutically acceptable salt,” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 2-19 (1977). The salts can be prepared in situ during the final isolation and purification of the compounds of the invention, or separately by reacting the free base function with a suitable organic acid. Examples of pharmaceutically acceptable salts include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentane-propionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

### Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intra-arterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectable.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to Van Devanter et al., U.S. Pat. No. 5,508,269 to Smith et al., and WO 98/43650 by Montgomery, all of which are incorporated herein by reference).

### Combination and Alternation Therapy

The compounds of the present invention may be used in combination with one or more antiviral therapeutic agents or anti-inflammatory agents useful in the prevention or treatment of viral diseases or associated pathophysiology. Thus, the compounds of the present invention and their salts, solvates, or other pharmaceutically acceptable derivatives thereof, may be employed alone or in combination with other antiviral or anti-inflammatory therapeutic agents. The compounds herein and pharmaceutically acceptable salts thereof may be used in combination with one or more other agents which may be useful in the prevention or treatment of respiratory disease, inflammatory disease, autoimmune disease, for example; anti-histamines, corticosteroids, (e.g., fluticasone propionate, fluticasone furoate, beclomethasone dipropionate, budesonide, ciclesonide, mometasone furoate, triamcinolone, flunisolide), NSAIDs, leukotriene modulators (e.g., montelukast, zafirlukast.pranlukast), tryptase inhibitors, IKK2 inhibitors, p38 inhibitors, Syk inhibitors, protease inhibitors such as elastase inhibitors, integrin antagonists (e.g., beta-2 integrin antagonists), adenosine A2a agonists, mediator release inhibitors such as sodium chromoglycate, 5-lipoxygenase inhibitors (zyflo), DP1 antagonists, DP2 antagonists, PI3K delta inhibitors, ITK inhibitors, LP (Iysophosphatidic) inhibitors or FLAP (5-lipoxygenase activating protein) inhibitors (e.g., sodium 3-(3-(tert-butylthio)-1-(4-(6-ethoxypyridin-3-yl)benzyl)-5-((5-ethylpyridin-2-yl)methoxy)-1H-indol-2-yl)-2,2-dimethylpropanoate), bronchodilators (e.g., muscarinic antagonists, beta-2 agonists), methotrexate, and similar agents; monoclonal antibody therapy such as anti-lgE, anti-TNF, anti-IL-5, anti-IL-6, anti-IL-12, anti-IL-1 and similar agents; cytokine receptor therapies e.g. etanercept and similar agents; antigen non-specific immunotherapies (e.g. interferon or other cytokines/chemokines, chemokine receptor modulators such as CCR3, CCR4 or CXCR2 antagonists, other cytokine/chemokine agonists or antagonists, TLR agonists and similar agents), suitable anti-infective agents including antibiotic agents, antifungal agents, antheimintic agents, antimalarial agents, antiprotozoal agents, antitubercuiosis agents, and antiviral agents, including those listed at https://www.drugs.com/drug-class/anti-infectives.html.

The compounds of the present invention and any other pharmaceutically active agent(s) may be administered together or separately and, when administered separately, administration may occur simultaneously or sequentially, in any order. The amounts of the compounds of the present invention and the other pharmaceutically active agent(s) and the relative timings of administration will be selected in order to achieve the desired combined therapeutic effect. The administration in combination of a compound of the present invention and salts, solvates, or other pharmaceutically acceptable derivatives thereof with other treatment agents may be achieved by concomitant administration in: (1) a unitary pharmaceutical composition including both compounds; or (2) separate pharmaceutical compositions each including one of the compounds.

In certain embodiments of the combination therapy, the additional therapeutic agent is administered at a lower dose and/or dosing frequency as compared to dose and/or dosing frequency of the additional therapeutic agent required to achieve similar results in treating or preventing of an 17β-HSD13 mediated disease or condition.

Although the invention has been described with respect to various preferred embodiments, it is not intended to be limited thereto, but rather those skilled in the art will recognize that variations and modifications may be made therein which are within the spirit of the invention and the scope of the appended claims.

### Therapeutic Activity

A therapeutically effective amount or dose of the compounds of the present invention may range from about 0.01 mg/Kg to about 500 mg/Kg, alternatively from about 1 to about 50 mg/Kg. Therapeutically effective amounts or doses will also vary depending on route of administration, as well as the possibility of co-usage with other agents.

According to the methods of treatment of the present invention, viral infections are treated or prevented in a patient such as a human or another animal by administering to the patient a therapeutically effective amount of a compound of the invention, in such amounts and for such time as is necessary to achieve the desired result.

By a “therapeutically effective amount” of a compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the compound described above may range from about 0.1 mg/Kg to about 500 mg/Kg, preferably from about 1 to about 50 mg/Kg. Effective doses will also vary depending on route of administration, as well as the possibility of co-usage with other agents. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this invention administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of this invention per day in single or multiple doses.

The compounds of the present invention described herein can, for example, be administered by injection, intravenously, intra-arterial, subdermally, intraperitoneally, intramuscularly, or subcutaneously; or orally, buccally, nasally, transmucosally, topically, in an ophthalmic preparation, or by inhalation, with a dosage ranging from about 0.1 to about 500 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with pharmaceutically excipients or carriers to produce a single dosage form will vary depending upon the host treated and the particular mode of administration. A typical preparation will contain from about 5% to about 95% active compound (w/w). Alternatively, such preparations may contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

When the compositions of this invention comprise a combination of a compound of Formula (I) described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed with one or more compounds of this invention in a single composition.

The “additional therapeutic or prophylactic agents” include but are not limited to, immune therapies (e.g. interferon), therapeutic vaccines, antifibrotic agents, anti-inflammatory agents such as corticosteroids or NSAIDs, bronchodilators such as beta-2 adrenergic agonists and xanthines (e.g. theophylline), mucolytic agents, anti-muscarinics, anti-leukotrienes, inhibitors of cell adhesion (e.g. ICAM antagonists), anti-oxidants (e.g. N-acetylcysteine), cytokine agonists, cytokine antagonists, lung surfactants and/or antimicrobial and anti-viral agents (e.g. ribavirin and amantidine). The compositions according to the invention may also be used in combination with gene replacement therapy.

### Abbreviations

Abbreviations which have been used in the descriptions of the schemes and the examples that follow are:
Alloc for allyloxycarbonyl;
Alloc-Cl for allyl chloroformate;
ASK1 for apoptosis signal-regulating kinase 1;
ATP for adenosine triphosphate;
Boc for tert-butyloxycarbonyl;
BOP-Cl for bis(2-oxo-3-oxazolidinyl)phosphinic chloride;
Cbz for benzyloxycarbonyl;
Cbz-Cl for benzyl chloroformate;
CDI for carbonyldiimidazole;
(COCl)₂ for oxalyl chloride;
DBU for 1,8-diazabicycloundec-7-ene;
DCC for N,N-dicyclohexylcarbodiimide;
1,2-DCE for 1,2-dichloroethane;
DCM for dichloromethane;
DIPEA or Hunig's base or i-Pr₂NEt for N,N-diisopropylethylamine;
DMAc for N,N-dimethylacetamide;
DMAP for N,N-dimethylaminopyridine;
DMF for N,N-dimethyl formamide;
EDC for 1-(3-diethylaminopropyl)-3-ethylcarbodiimide hydrochloride;
EGTA for ethylene glycol-bis(2-aminoethylether)-N,N,N′,N′-tetraacetic acid;
ESI for electrospray ionization;
Et₃N or TEA for triethylamine;
Et₂O for diethylether;
EtOAc for ethyl acetate;
Ghosez's Reagent for 1-chloro-N,N,2-trimethyl-1-propenylamine;
HATU for 1-[bis(dimethylamino)methylene]-1H-1,2,3-triazolo[4,5-b]pyridinium 3-oxid hexafluorophosphate;
HEPES for 4-(2-Hydroxyethyl)piperazine-1-ethanesulfonic acid, N-(2-Hydroxyethyl)piperazine-N′-(2-ethanesulfonic acid);
IC₅₀ for half maximal inhibitory concentration;
KOt-Bu for potassium tert-butoxide;
LCMS for liquid chromatography-mass spectrometry;
MeCN for acetonitrile;
MTBE for methyl tert-butyl ether;
m/z for mass-to-charge ratio;
NaOt-Bu for sodium tert-butoxide;
NMP for 1-methyl-2-pyrrolidinone;
NMR for nuclear magnetic resonance spectroscopy;
—OMs or mesylate for methanesulfonate;
—OTf or triflate for trifluoromethanesulfonate;
—OTs or tosylate for para-toluenesulfonate;
Pd₂(dba)₃ for tris(dibenzylideneacetone)dipalladium(0);
P(o-tolyl)₃ for tri(o-tolyl)phosphine;
PyAOP for 7-azabenzotriazol-1-yloxy)tripyrrolidinophosphonium hexafluorophosphate;
PyBOP for benzotriazol-1-yl-oxytripyrrolidinophosphonium hexafluorophosphate;
STK3 for serine/threonine-protein kinase 3
TEA for triethylamine;
THF for tetrahydrofuran.

### Synthetic Methods

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Although the invention has been described with respect to various preferred embodiments, it is not intended to be limited thereto, but rather those skilled in the art will recognize that variations and modifications may be made therein which are within the spirit of the invention and the scope of the appended claims.

The compounds and processes of the present invention will be better understood in connection with the following synthetic schemes that illustrate the methods by which the compounds of the invention may be prepared, which are intended as an illustration only and not to limit the scope of the invention.

As shown in Scheme 1, in a one-pot fashion, the compound of Formula (I) can be prepared from condensation reaction of the amino ester compound (1) prepared according to literature (New inhibitors of 17b-hydroxysteroid dehydrogenase type 1, Molecular and Cellular Endocrinology 2006, 248, 192-198, Josef Messinger, Leena Hirvela, Pasi Koskimies, Bettina Husen, Lauri Kangas, Olli Pentikainen, Pauli Saarenketo and Hubert Thole) and the amide compound (2) in the presence of POCl₃, SOCl₂ or PCl₅, wherein, R₁, R₂, R₃, R₄, R₅, R₆ and M are as previously defined. Thus, a mixture of amino ester compound (1) and amide compound (2) in an aprotic solvent is treated with POCl₃, SOCl₂ or PCl₅ to form compound of Formula (I). The aprotic solvent can be, such as, but not limited to, THF, DCE and DMF. The reaction temperature is from 0° C. to 140° C.

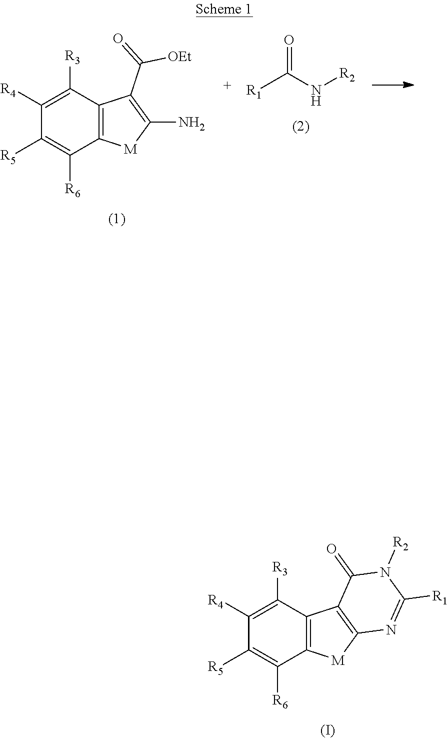

Alternatively, as shown in Scheme 2, the compound of Formula (I) can also be prepared via a stepwise fashion, wherein, R₁, R₂, R₃, R₄, R₅, R₆, and M are as previously defined. The amide compound (2) can react with POCl₃, SOCl₂ or PCl₅ in an aprotic solvent such as DCM, DCE, THF, or DMF to form imine chloride (3) at 0° C.˜80° C. followed by reacting with the amino ester compound (1) to give the cyclized compound (I). The reaction temperature is from 0° C. to 140° C.

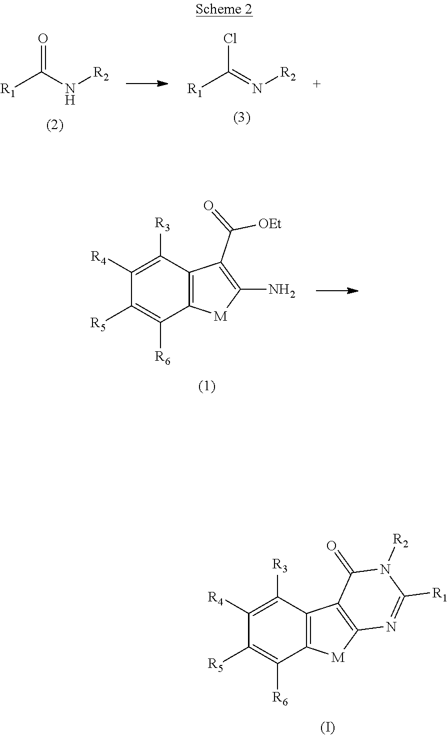

Alternatively, as shown in Scheme 3, the compound of Formula (I) can also be prepared by a transition metal/phosphine ligand complex catalyzed coupling reaction between the chloropyrimidone compound (7) and an organometallic reagent such as a boronic acid or related boron reagent partner (8), wherein, R₁, R₂, R₃, R₄, R₅, R₆ and M are as previously defined. The catalyst used in this reaction can be, but not limited to bis(triphenylphosphine)palladium(II) chloride. The base used in this reaction can be, but is not limited to, cesium carbonate. Compound (1) in an aprotic solvent is first treated with amine compound (4) in the presence of suitable coupling reagent and organic base to afford the urea compound (5). The suitable coupling reagent can be, such as, but not limited to, CDI, triphosgene or ethyl chloroformate and the organic base can be, such as, but not limited to, DBU, DIPEA or TEA. The aprotic solvent can be, such as, but not limited to, MeCN, THF, DCE or DMF. The reaction temperature is from 0° C. to 80° C. Then urea compound (5) is treated with suitable inorganic base in alcohol solvents at elevated temperature to provide the pyrimidinedione compound (6). The inorganic bases can be, such as, but not limited to, NaOMe, NaOEt, or NaOtBu. The alcohol solvent can be, such as, but not limited to, MeOH, EtOH or tBuOH. The temperature is from 40° C. to 80° C. The chloropyrimidone compound (7) is prepared from pyrimidinedione compound (6) in the presence of suitable chlorinating reagent in an aprotic solvent at elevated temperature. The suitable chlorinating reagent can be such as, but not limited to POCl₃. The aprotic solvent can be, such as, but not limited to, DCE. The reaction temperature is from 80° C. to 140° C. The chloropyrimidone compound (7) reacts with a boronic acid partner or related boron reagents (8) catalyzed by a transition metal/phosphine ligand complex in mixed solvents mixture. The solvent in this coupling reaction can be, but not limited to DME/H₂O, dioxane/H₂O, toluene/H₂O, etc. The reaction temperature is from 0° C. to 140° C.

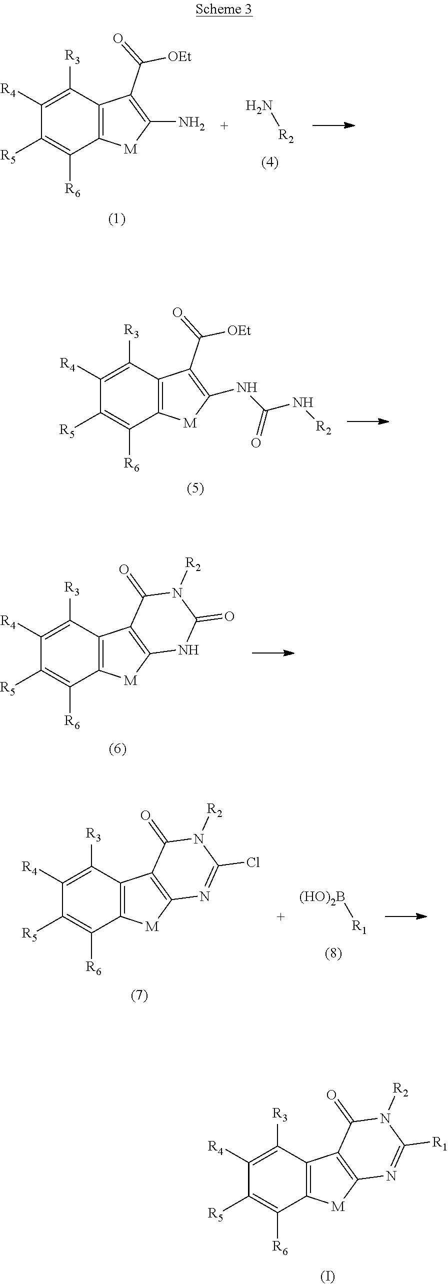

As shown in Scheme 4, the compound of Formula (IV) could be synthesized by substitution reaction between compound (9) and R₉-LG in aprotic solvent and in the presence of base, wherein, R₁, R₂, R₃, R₄, R₅, R₉ and M are as previously defined. The base can be organic bases or inorganic bases, but not limited to pyridine, DIPEA, DMAP, Cs₂CO₃, NaH, etc. The aprotic solvent can be, such as, but not limited to, pyridine, MeCN, THF, DCE or DMF. The reaction temperature is from 0° C. to 80° C.

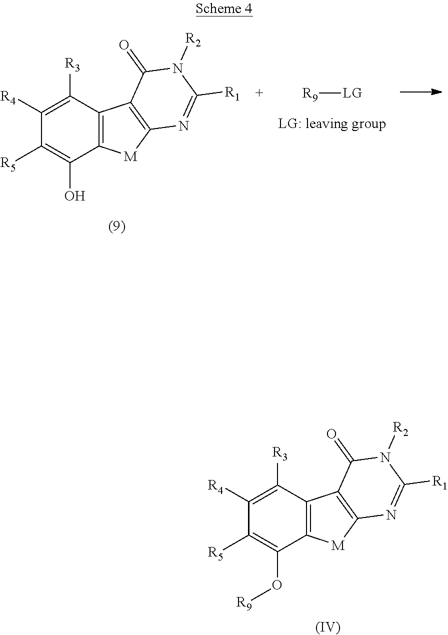

As shown in Scheme 5, the compound of Formula (I) could be synthesized by transition metal catalyzed reaction between compound (10) and (11). Wherein, R₁, R₂, R₃, R₄, R₅, R₆ and M are as previously defined. X is a pseudohalide such as —OTf, —OTs, —OMs, —N₂⁺ or a halogen such as Cl, Br, I; Xa is a functional group suitable for metal catalyzed cross couplings such as boronic acid/ester, organic tin, organic zinc species. The compounds (10) and (11) under standard metal-catalyzed coupling conditions (e.g., using a palladium catalyst) in a suitable solvent (e.g., dioxane, water, etc.), optionally under an inert atmosphere, to provide compounds of formula (I). (For metal-catalyzed coupling reactions see: N. Miyaura, S. L. Buchwald, etc. Cross-Coupling Reactions: A Practical Guide (Topics in Current Chemistry, 219), Springer (2002); A de Meijere, S. Brase, M. Oestreich, Metal Catalyzed Cross-Coupling Reactions and More, Wiley-VCR (2014); I. D. Kostas, Suzuki-Miyaura Cross-Coupling Reaction and Potential Applications, Mdpi AG (2017).

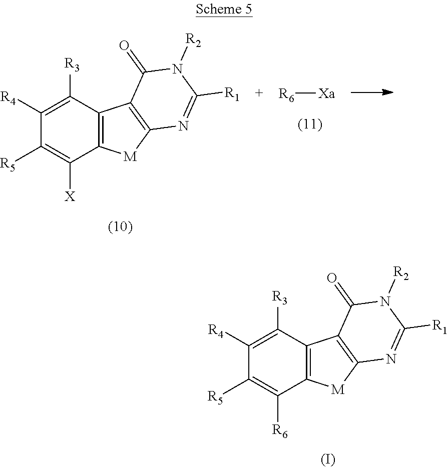

As shown in Scheme 6, the compound of Formula (I) could be synthesized by transition metal catalyzed reaction between compound (12) and (13). Wherein, R₁, R₂, R₃, R₄, R₅, R₆ and M are as previously defined. X is a halogen such as Cl, Br, I or a pseudohalide such as —OTf, —OTs, —OMs, —N₂⁺; Xa is a functional group suitable for metal catalyzed cross couplings such as boronic acid/ester, organic tin, organic zinc species. The compounds (12) and (13) under standard metal-catalyzed coupling conditions (e.g., using catalysts such as palladium, copper, nickel, etc.) in a suitable solvent (e.g., dioxane, water, etc.), optionally under an inert atmosphere, to provide compounds of formula (I).

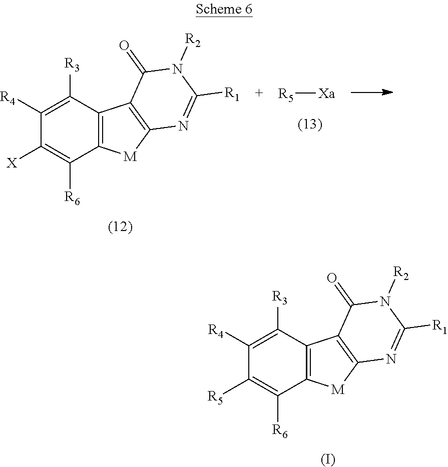

As shown in Scheme 7, the compound of Formula (I) could be synthesized by Claisen rearrangement starting from compound (14) by heating or transition metal catalyzed approach. Wherein, R₁, R₂, R₃, R₄, R₅, R₆ and M are as previously defined. The ortho-allyl phenol (15) could be further converted into compounds of formula (I) via traditional functional group transformations.

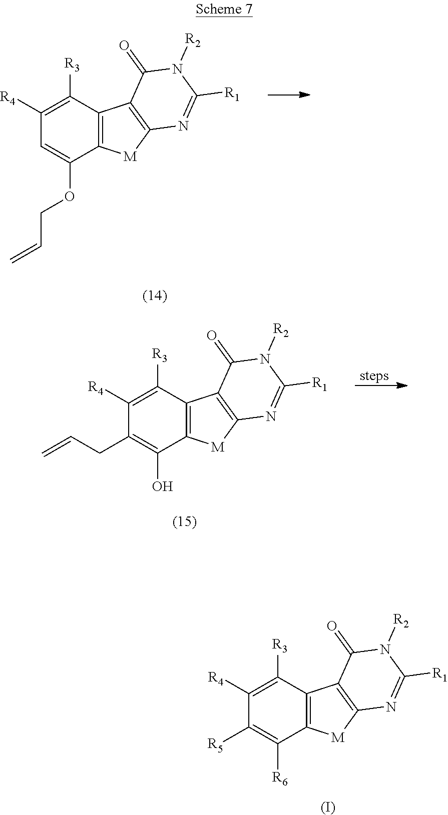

As shown in Scheme 8, the compound of Formula (I) can be synthesized by electrophilic fluorination starting from compound (16) by reacting with a varieties of N-F reagents, such as NFSI, N-fluoropyridinium salts, SelectFluor, NFOBS, etc. Wherein, R₁, R₂, R₃, R₄, R₅, R₆ and M are as previously defined. The fluorinated phenols (17, 18, 19) could be further converted into compounds of formula (I) via traditional functional group transformations.

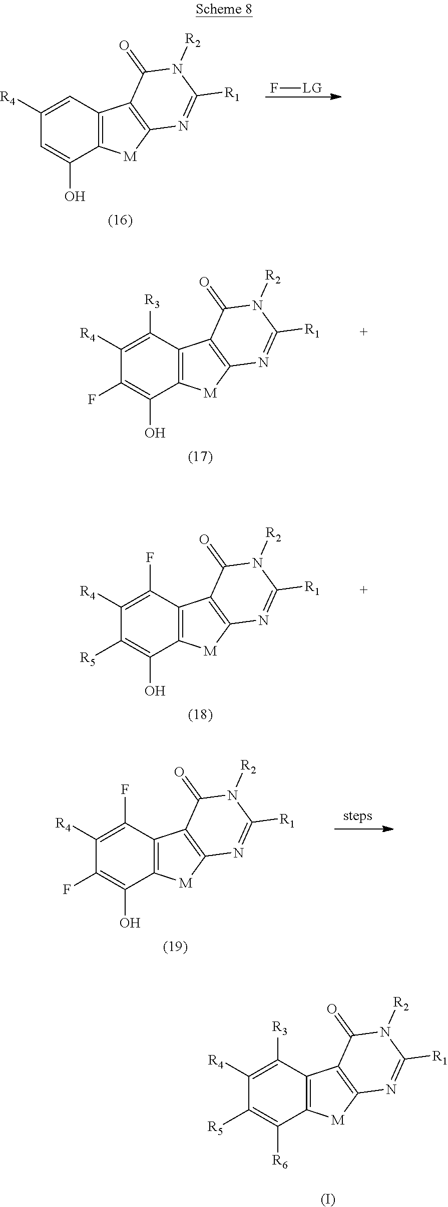

Alternatively, as shown in Scheme 9, the compound of Formula (I) can also be prepared by an oxidative ring closing reaction via compound (23) mediated by an oxidant. Wherein, R₁, R₂, R₃, R₄, R₅, R₆ and M are as previously defined. The oxidant used in this reaction can be, but not limited to iodine. Compound (20) in an aprotic solvent is first treated with amine compound (4) in the presence of a suitable coupling reagent and organic base to afford the amide compound (21). The suitable coupling reagent can be, such as, but not limited to, HATU, HBTU or EDCI and the organic base can be, such as, but not limited to, DIPEA or TEA. Then amide compound (21) is treated with an aldehyde compound (22) to provide the imine amide compound (23) then proceed to the oxidative ring closing reaction mediated by an oxidant such as iodine. The reaction can be carried out via a stepwise process or in a one-pot fashion.

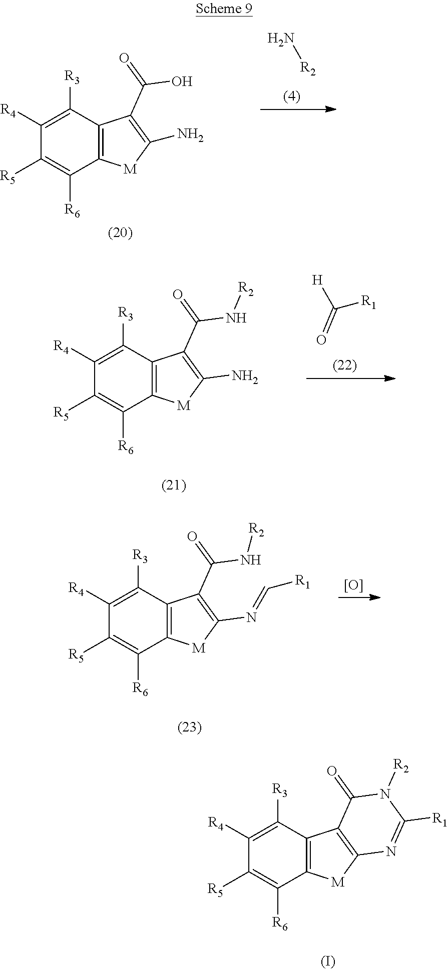

### EXAMPLES

The compounds and processes of the present invention will be better understood in connection with the following examples, which are intended as an illustration only and not limiting of the scope of the invention. Starting materials were either available from a commercial vendor or produced by methods well known to those skilled in the art.

### Synthesis of methyl 2-(2-cyclopropyl-4-methoxyphenyl)-3-(oxazol-5-ylmethyl)-4-oxo-3,4-dihydrobenzo[4,5]thieno[2,3-d]pyrimidine-8-carboxylate (example 1)

### Example 1

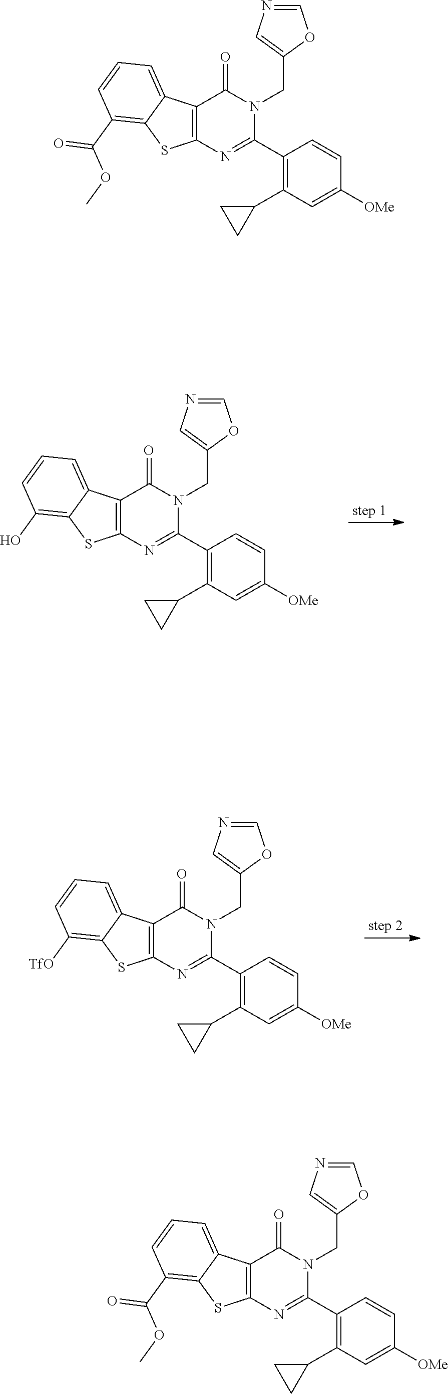 [SMILES: COC(=O)c1cccc2c1sc1nc(-c3ccc(OC)cc3C3CC3)n(Cc3cnco3)c(=O)c12 ; COc1ccc(-c2nc3sc4c(O)cccc4c3c(=O)n2Cc2cnco2)c(C2CC2)c1 ; COc1ccc(-c2nc3sc4c(OS(=O)(=O)C(F)(F)F)cccc4c3c(=O)n2Cc2cnco2)c(C2CC2)c1 ; "step 2" ; COC(=O)c1cccc2c1sc1nc(-c3ccc(OC)cc3C3CC3)n(Cc3cnco3)c(=O)c12]

### Step 1

To a 250 mL vial containing 2-(2-cyclopropyl-4-methoxyphenyl)-8-hydroxy-3-(oxazol-5-ylmethyl)benzo[4,5]thieno[2,3-d]pyrimidin-4(3H)-one (0.98 g, 2.200 mmol) which was prepared according to method (U.S. Provisional Application No. 63/335,840) was added Pyridine (29.3 ml) and the solution was cooled to 0° C. under N₂ followed by addition of trifluoromethanesulfonic anhydride (1.115 ml, 6.60 mmol) dropwise. The reaction was stirred at 0° C.˜rt overnight and was complete by LC/MS and TLC. Diluted with MTBE, washed with half Sat. NaHCO₃ once, water twice, Sat. NaHCO₃ once, and finally brine once. Dried, filtered, concentrated to give 2-(2-cyclopropyl-4-methoxyphenyl)-3-(oxazol-5-ylmethyl)-4-oxo-3,4-dihydrobenzo[4,5]thieno[2,3-d]pyrimidin-8-yl trifluoromethanesulfonate (1.14 g, 90% yield) as a light orange foam. LC-MS observed [M+H], 578.07.

### Step 2

To a 2-dram vial were added 2-(2-cyclopropyl-4-methoxyphenyl)-3-(oxazol-5-ylmethyl)-4-oxo-3,4-dihydrobenzo[4,5]thieno[2,3-d]pyrimidin-8-yl trifluoromethanesulfonate (500 mg, 0.866 mmol), dppf (48.0 mg, 0.087 mmol), palladium(II) acetate (9.72 mg, 0.043 mmol), and DMF (1.5 mL) respectively and the vial was vacuumed and backfilled with N₂. TEA (0.241 mL, 1.731 mmol) and MeOH (0.84 mL, 20.76 mmol) were added. The mixture was degassed and backfilled with carbon monoxide via a balloon. The brown mixture was heated at 65° C. for 15 h. Diluted with MTBE, washed with water twice and brine once. Dried, filtered, concentrated to give a brown solid ˜500 mg. Purified by CombiFlash (40 g SiO₂ gold, Ace/c-Hex: 0˜40%) to give methyl 2-(2-cyclopropyl-4-methoxyphenyl)-3-(oxazol-5-ylmethyl)-4-oxo-3,4-dihydrobenzo[4,5]thieno[2,3-d]pyrimidine-8-carboxylate (317 mg, 75% yield) as a white solid. LC-MS observed [M+H], 488.13. ¹H NMR (400 MHz, DMSO) δ 8.84 (dt, J=7.9, 1.1 Hz, 1H), 8.27-8.19 (m, 2H), 7.79 (td, J=7.8, 0.9 Hz, 1H), 7.38 (d, J=8.5 Hz, 1H), 6.88 (dd, J=8.5, 2.4 Hz, 1H), 6.86 (s, 1H), 6.49 (d, J=2.4 Hz, 1H), 5.42 (d, J=15.9 Hz, 1H), 5.13 (d, J=15.9 Hz, 1H), 4.00 (s, 3H), 3.82 (s, 3H), 1.65-1.54 (m, 1H), 0.87-0.74 (m, 3H), 0.67-0.58 (m, 1H).

### Synthesis of 2-(2-cyclopropyl-4-methoxyphenyl)-3-(oxazol-5-ylmethyl)-4-oxo-3,4-dihydrobenzo[4,5]thieno[2,3-d]pyrimidine-8-carboxylic acid (example 2)

### Example 2

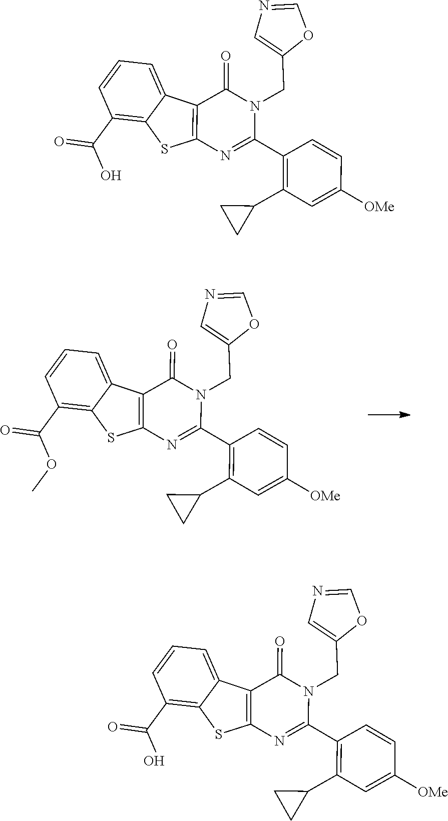 [SMILES: COc1ccc(-c2nc3sc4c(C(=O)O)cccc4c3c(=O)n2Cc2cnco2)c(C2CC2)c1 ; COC(=O)c1cccc2c1sc1nc(-c3ccc(OC)cc3C3CC3)n(Cc3cnco3)c(=O)c12 ; COc1ccc(-c2nc3sc4c(C(=O)O)cccc4c3c(=O)n2Cc2cnco2)c(C2CC2)c1]

To a 20 mL vial were added methyl 2-(2-cyclopropyl-4-methoxyphenyl)-3-(oxazol-5-ylmethyl)-4-oxo-3,4-dihydrobenzo[4,5]thieno[2,3-d]pyrimidine-8-carboxylate (203 mg, 0.416 mmol), THF (5.55 mL), and a solution of LiOH (100 mg, 4.16 mmol) in water (2.78 mL) and the mixture was stirred at rt for 75 min and then at 40° C. for another 3 h. Diluted with DCM, washed with 10% citric acid and brine. Dried, filtered, concentrated to give 2-(2-cyclopropyl-4-methoxyphenyl)-3-(oxazol-5-ylmethyl)-4-oxo-3,4-dihydrobenzo[4,5]thieno[2,3-d]pyrimidine-8-carboxylic acid as an off-white solid (210.1 mg, quan. yield). LC-MS observed [M+H], 474.11. ¹H NMR (400 MHz, DMSO) δ 13.80 (bs, 1H), 8.80 (dd, J=8.0, 1.3 Hz, 1H), 8.21 (s, 1H), 8.19 (dd, J=8.0, 1.3 Hz, 1H), 7.75 (t, J=7.7 Hz, 1H), 7.38 (d, J=8.5 Hz, 1H), 6.88 (dd, J=8.4, 2.4 Hz, 1H), 6.85 (s, 1H), 6.48 (d, J=2.5 Hz, 1H), 5.42 (d, J=15.9 Hz, 1H), 5.12 (d, J=15.9 Hz, 1H), 3.81 (s, 3H), 1.68-1.52 (m, 1H), 0.92-0.71 (m, 3H), 0.62 (d, J=7.0 Hz, 1H).

### Synthesis of 2-(2-cyclopropyl-4-methoxyphenyl)-3-(oxazol-5-ylmethyl)-4-oxo-3,4-dihydrobenzo[4,5]thieno[2,3-d]pyrimidine-8-carboxamide (example 3)

### Example 3

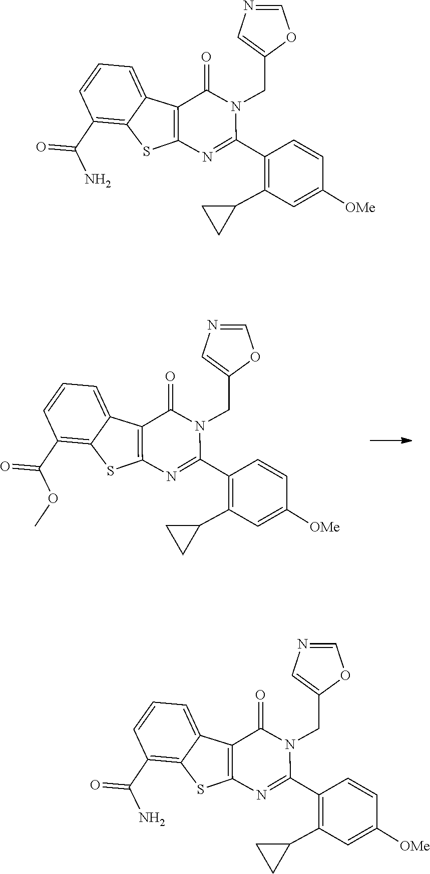 [SMILES: COc1ccc(-c2nc3sc4c(C(N)=O)cccc4c3c(=O)n2Cc2cnco2)c(C2CC2)c1 ; COC(=O)c1cccc2c1sc1nc(-c3ccc(OC)cc3C3CC3)n(Cc3cnco3)c(=O)c12 ; COc1ccc(-c2nc3sc4c(C(N)=O)cccc4c3c(=O)n2Cc2cnco2)c(C2CC2)c1]

To a 1-dram vial were added methyl 2-(2-cyclopropyl-4-methoxyphenyl)-3-(oxazol-5-ylmethyl)-4-oxo-3,4-dihydrobenzo[4,5]thieno[2,3-d]pyrimidine-8-carboxylate (30 mg, 0.062 mmol) and 7N ammonia in MeOH (2 mL, 14.00 mmol) and the mixture was heated at 50° C. for 6 h. Concentrated and purified by Prep HPLC (ACN/H₂O: 30˜75%) to give 2-(2-cyclopropyl-4-methoxyphenyl)-3-(oxazol-5-ylmethyl)-4-oxo-3,4-dihydrobenzo[4,5]thieno[2,3-d]pyrimidine-8-carboxamide (20.4 mg, 70.2% yield). LC-MS observed [M+H], 473.13. ¹H NMR (400 MHz, DMSO) δ 8.74 (d, J=7.8 Hz, 1H), 8.42 (s, 1H), 8.21 (s, 1H), 8.19 (d, J=7.6 Hz, 1H), 7.81 (s, 1H), 7.72 (t, J=7.8 Hz, 1H), 7.38 (d, J=8.5 Hz, 1H), 6.88 (dd, J=8.6, 2.5 Hz, 1H), 6.84 (s, 1H), 6.48 (d, J=2.5 Hz, 1H), 5.41 (d, J=15.9 Hz, 1H), 5.11 (d, J=15.9 Hz, 1H), 3.81 (s, 3H), 1.63-1.52 (m, 1H), 0.90-0.74 (m, 3H), 0.67-0.56 (m, 1H).

### Synthesis of N-(2-(2-cyclopropyl-4-methoxyphenyl)-3-(oxazol-5-ylmethyl)-4-oxo-3,4-dihydrobenzo[4,5]thieno[2,3-d]pyrimidin-8-yl)acetamide (example 4) and N-acetyl-N-(2-(2-cyclopropyl-4-methoxyphenyl)-3-(oxazol-5-ylmethyl)-4-oxo-3,4-dihydrobenzo[4,5]thieno[2,3-d]pyrimidin-8-yl)acetamide (example 5)

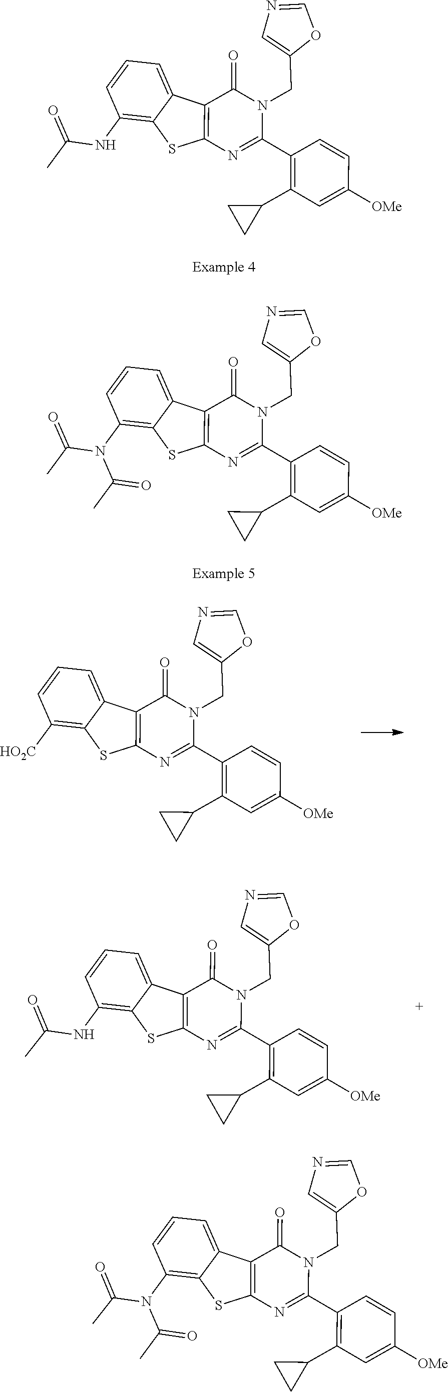 [SMILES: COc1ccc(-c2nc3sc4c(NC(C)=O)cccc4c3c(=O)n2Cc2cnco2)c(C2CC2)c1 ; COc1ccc(-c2nc3sc4c(N(C(C)=O)C(C)=O)cccc4c3c(=O)n2Cc2cnco2)c(C2CC2)c1 ; COc1ccc(-c2nc3sc4c(C(=O)O)cccc4c3c(=O)n2Cc2cnco2)c(C2CC2)c1 ; COc1ccc(-c2nc3sc4c(NC(C)=O)cccc4c3c(=O)n2Cc2cnco2)c(C2CC2)c1 ; COc1ccc(-c2nc3sc4c(N(C(C)=O)C(C)=O)cccc4c3c(=O)n2Cc2cnco2)c(C2CC2)c1]

To a 1-dram vial were added 2-(2-cyclopropyl-4-methoxyphenyl)-3-(oxazol-5-ylmethyl)-4-oxo-3,4-dihydrobenzo[4,5]thieno[2,3-d]pyrimidine-8-carboxylic acid (30 mg, 0.063 mmol), DMF (1 ml), and the solution was cooled to 0° C. under N₂ followed by addition of DIPEA (26.6 μl, 0.152 mmol) and Diphenylphosphoryl azide (16.38 μl, 0.076 mmol). After stirred at 0° C. for 2 h, the solution was heated at 100° C. for 4 h and then acetic anhydride (120 μl, 1.267 mmol) and acetic acid (36.3 μl, 0.634 mmol) were added. The solution was stirred at 100° C. for 5 h and then cooled to rt. Diluted with DCM and washed with Sat. NaHCO₃ and brine respectively. The organic layer was separated, dried, filtered, concentrated, and purified by Prep HPLC (30˜75% ACN/H₂O) to give N-(2-(2-cyclopropyl-4-methoxyphenyl)-3-(oxazol-5-ylmethyl)-4-oxo-3,4-dihydrobenzo[4,5]thieno[2,3-d]pyrimidin-8-yl)acetamide (10.3 mg, 33.2% yield); LC-MS observed [M+H], 487.18. ¹H NMR (400 MHz, CDCl₃) δ 8.53 (d, J=7.9 Hz, 1H), 7.83 (d, J=7.9 Hz, 1H), 7.75 (s, 1H), 7.56 (t, J=7.9 Hz, 1H), 7.30-7.22 (m, 2H), 6.88-6.81 (m, 2H), 6.49 (d, J=2.5 Hz, 1H), 5.62 (d, J=15.4 Hz, 1H), 5.09 (d, J=15.4 Hz, 1H), 3.86 (s, 3H), 2.31 (s, 3H), 1.61-1.51 (m, 1H), 0.98-0.79 (m, 3H), 0.71-0.63 (m, 1H). N-acetyl-N-(2-(2-cyclopropyl-4-methoxyphenyl)-3-(oxazol-5-ylmethyl)-4-oxo-3,4-dihydrobenzo[4,5]thieno[2,3-d]pyrimidin-8-yl)acetamide (5.2 mg, 15.8% yield) was obtained as well. LC-MS observed [M+H], 529.15. ¹H NMR (400 MHz, CDCl₃) δ 8.75 (dd, J=8.0, 1.1 Hz, 1H), 7.75 (s, 1H), 7.67 (t, J=7.9 Hz, 1H), 7.33 (dd, J=7.7, 1.1 Hz, 1H), 7.23 (d, J=8.5 Hz, 1H), 6.89-6.81 (m, 2H), 6.50 (d, J=2.5 Hz, 1H), 5.62 (d, J=15.3 Hz, 1H), 5.10 (d, J=15.3 Hz, 1H), 3.86 (s, 3H), 2.40 (s, 3H), 2.31 (s, 3H), 1.61-1.48 (m, 1H), 0.98-0.79 (m, 3H), 0.72-0.62 (m, 1H).

### Synthesis of 8-amino-2-(2-cyclopropyl-4-methoxyphenyl)-3-(oxazol-5-ylmethyl)benzo[4,5]thieno[2,3-d]pyrimidin-4(3H)-one (example 6)

### Example 6

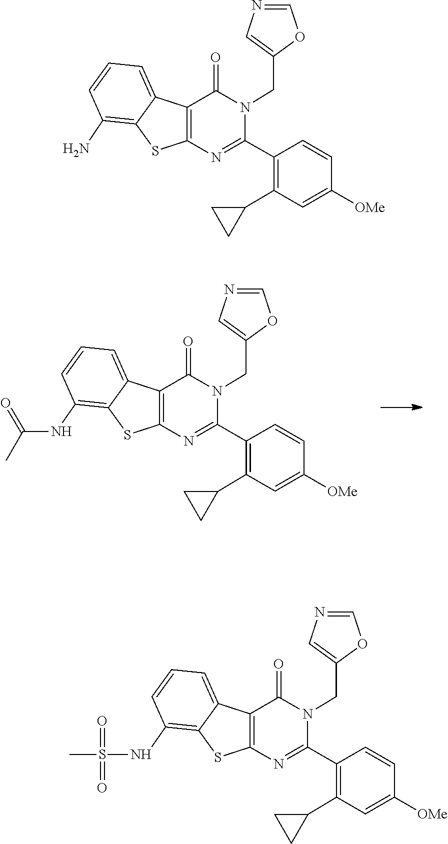 [SMILES: COc1ccc(-c2nc3sc4c(N)cccc4c3c(=O)n2Cc2cnco2)c(C2CC2)c1 ; COc1ccc(-c2nc3sc4c(NC(C)=O)cccc4c3c(=O)n2Cc2cnco2)c(C2CC2)c1 ; COc1ccc(-c2nc3sc4c(NS(C)(=O)=O)cccc4c3c(=O)n2Cc2cnco2)c(C2CC2)c1]

6N HCl (1 mL, 6.00 mmol) was added to a solution of N-(2-(2-cyclopropyl-4-methoxyphenyl)-3-(oxazol-5-ylmethyl)-4-oxo-3,4-dihydrobenzo[4,5]thieno[2,3-d]pyrimidin-8-yl)acetamide (7.0 mg, 0.014 mmol) in MeOH (2 mL) and the mixture was heated at 65° C. for 2 h and then 50° C. overnight. Concentrated in vacuo to give 8-amino-2-(2-cyclopropyl-4-methoxyphenyl)-3-(oxazol-5-ylmethyl)benzo[4,5]thieno[2,3-d]pyrimidin-4(3H)-one (7.0 mg, quan. yield) as a white foam. LC-MS observed [M+H], 445.13. ¹H NMR (400 MHz, CDCl₃) δ 8.21 (d, J=7.9 Hz, 1H), 7.75 (s, 1H), 7.42 (t, J=7.8 Hz, 1H), 7.26 (d, J=8.0 Hz, 1H), 6.92-6.78 (m, 3H), 6.49 (d, J=2.5 Hz, 1H), 5.63 (d, J=15.3 Hz, 1H), 5.08 (d, J=15.4 Hz, 1H), 3.86 (s, 3H), 1.62-1.52 (m, 1H), 1.27 (s, 2H), 0.98-0.79 (m, 3H), 0.72-0.62 (m, 1H).

### Synthesis of N-(2-(2-cyclopropyl-4-methoxyphenyl)-3-(oxazol-5-ylmethyl)-4-oxo-3,4-dihydrobenzo[4,5]thieno[2,3-d]pyrimidin-8-yl)methanesulfonamide (example 7)

### Example 7

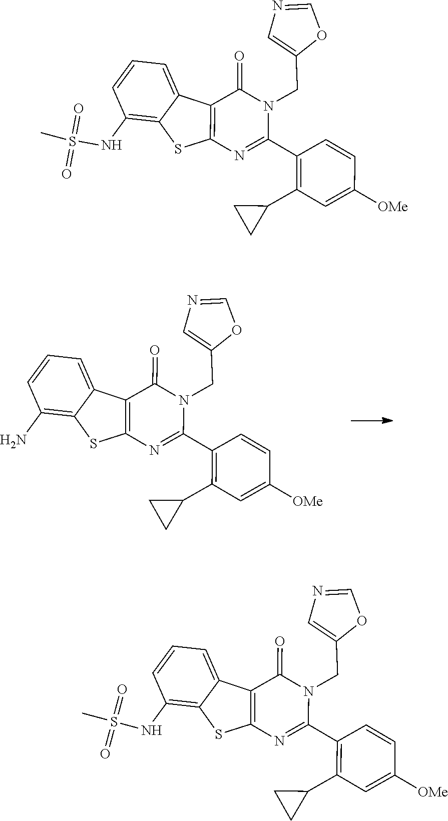 [SMILES: COc1ccc(-c2nc3sc4c(NS(C)(=O)=O)cccc4c3c(=O)n2Cc2cnco2)c(C2CC2)c1 ; COc1ccc(-c2nc3sc4c(N)cccc4c3c(=O)n2Cc2cnco2)c(C2CC2)c1 ; COc1ccc(-c2nc3sc4c(NS(C)(=O)=O)cccc4c3c(=O)n2Cc2cnco2)c(C2CC2)c1]

To a 2-dram vial were added 8-amino-2-(2-cyclopropyl-4-methoxyphenyl)-3-(oxazol-5-ylmethyl)benzo[4,5]thieno[2,3-d]pyrimidin-4(3H)-one (7.0 mg, 0.014 mmol), pyridine (3 mL), and methanesulfonyl chloride (5.41 mg, 0.047 mmol) respectively and the reaction was stirred at 0° C.˜rt overnight. The crude reaction mixture was diluted with DCM, washed with sat. NaHCO₃ solution and brine. Dried, filtered, concentrated, purified by Prep TLC (40% ACE/c-Hex) to give N-(2-(2-cyclopropyl-4-methoxyphenyl)-3-(oxazol-5-ylmethyl)-4-oxo-3,4-dihydrobenzo[4,5]thieno[2,3-d]pyrimidin-8-yl)methanesulfonamide (2.7 mg, 32.8% yield) as a white solid. LC-MS observed [M+H], 523.11. ¹H NMR (400 MHz, CDCl₃) δ 8.63 (dd, J=6.3, 2.8 Hz, 1H), 7.76 (s, 1H), 7.63 (s, 1H), 7.61 (d, J=3.6 Hz, 1H), 7.25 (d, J=8.5 Hz, 1H), 6.91-6.82 (m, 2H), 6.50 (d, J=2.4 Hz, 1H), 6.48 (s, 1H), 5.63 (d, J=15.3 Hz, 1H), 5.11 (d, J=15.3 Hz, 1H), 3.87 (s, 3H), 3.10 (s, 3H), 1.62-1.50 (m, 1H), 0.98-0.80 (m, 3H), 0.73-0.63 (m, 1H).

### Synthesis of 2-(2-cyclopropyl-4-methoxyphenyl)-8-methoxy-3-(oxazol-5-ylmethyl)benzo[4,5]thieno[2,3-d]pyrimidin-4(3H)-one (example 8)

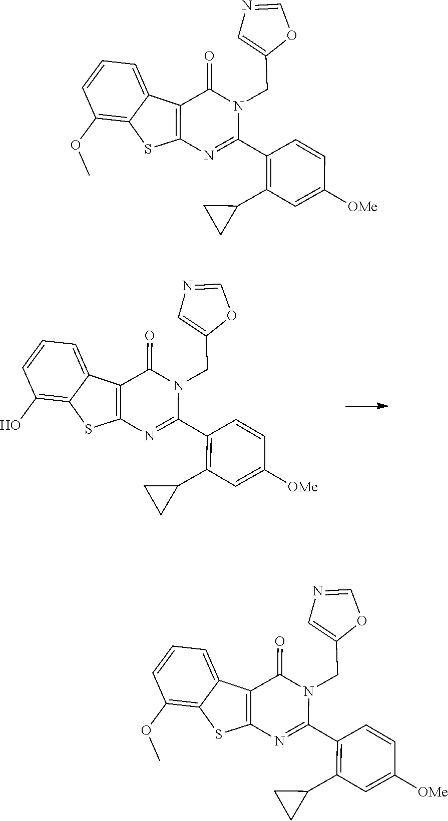 [SMILES: COc1ccc(-c2nc3sc4c(OC)cccc4c3c(=O)n2Cc2cnco2)c(C2CC2)c1 ; COc1ccc(-c2nc3sc4c(O)cccc4c3c(=O)n2Cc2cnco2)c(C2CC2)c1 ; COc1ccc(-c2nc3sc4c(OC)cccc4c3c(=O)n2Cc2cnco2)c(C2CC2)c1]

To a 2-dram vial were added 2-(2-cyclopropyl-4-methoxyphenyl)-8-hydroxy-3-(oxazol-5-ylmethyl)benzo[4,5]thieno[2,3-d]pyrimidin-4(3H)-one (100 mg, 0.224 mmol), DMF (2.245 mL), and the yellow solution was cooled to 0° C. under N₂ followed by addition of sodium hydride (13.47 mg, 0.337 mmol, 60% in mineral oil). After stirred at 0° C. for 40 min, an amber solution was observed. Iodomethane (23.76 μl, 0.382 mmol) was added dropwise and the color of the solution became orange. Stirred at 0° C. to rt for 3 h. Diluted with MTBE, washed with water twice and brine once. Dried, filtered, concentrated to give a yellow solid 104 mg. Purified by CombiFlash (Ace/cyclohexane: 0˜40%) to give 2-(2-cyclopropyl-4-methoxyphenyl)-8-methoxy-3-(oxazol-5-ylmethyl)benzo[4,5]thieno[2,3-d]pyrimidin-4(3H)-one (88.1 mg, 85% yield) as a white solid. LC-MS observed [M+H], 460.13. ¹H NMR (400 MHz, CDCl₃) δ 8.28 (dd, J=8.1, 0.8 Hz, 1H), 7.74 (s, 1H), 7.50 (t, J=8.0 Hz, 1H), 7.25 (d, J=8.5 Hz, 1H), 6.94 (dd, J=8.1, 0.9 Hz, 1H), 6.87-6.80 (m, 2H), 6.48 (d, J=2.5 Hz, 1H), 5.61 (d, J=15.4 Hz, 1H), 5.07 (dd, J=15.3, 0.9 Hz, 1H), 4.02 (s, 3H), 3.84 (s, 3H), 1.62-1.52 (m, 1H), 0.96-0.77 (m, 3H), 0.70-0.62 (m, 1H).

### Synthesis of 2-(2-cyclopropyl-4-methoxyphenyl)-8-(oxazol-2-yl)-3-(oxazol-5-ylmethyl)benzo[4,5]thieno[2,3-d]pyrimidin-4(3H)-one (example 9) and 8-butyl-2-(2-cyclopropyl-4-methoxyphenyl)-3-(oxazol-5-ylmethyl)benzo[4,5]thieno[2,3-d]pyrimidin-4(3H)-one

### Example 10

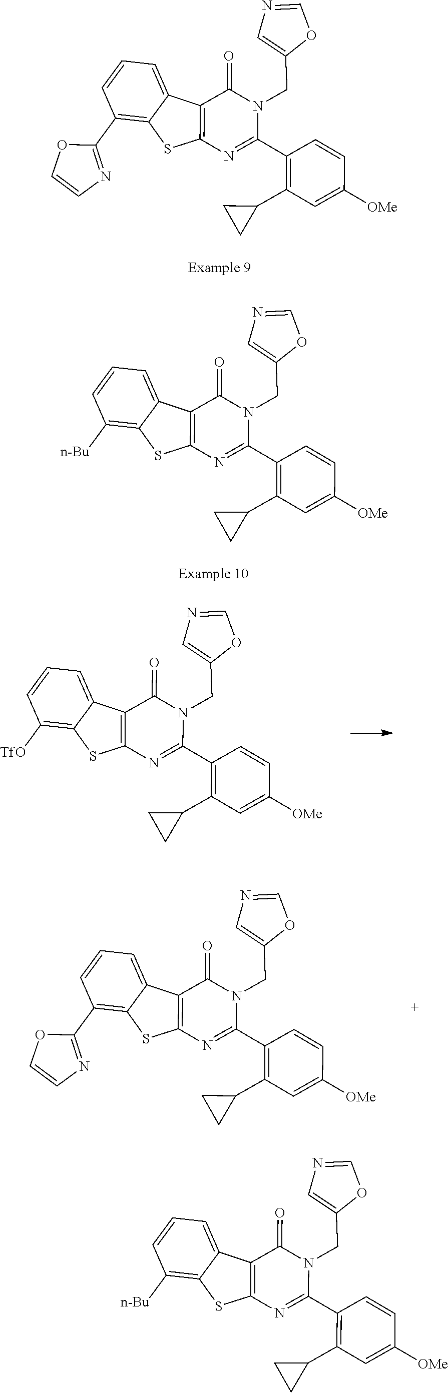

To a 2 mL microwave vial were added 2-(2-cyclopropyl-4-methoxyphenyl)-3-(oxazol-5-ylmethyl)-4-oxo-3,4-dihydrobenzo[4,5]thieno[2,3-d]pyrimidin-8-yl trifluoromethanesulfonate (50 mg, 0.087 mmol), 2-(tributylstannyl)oxazole (54.4 μl, 0.260 mmol), PdCl₂(dppf) (9.50 mg, 0.013 mmol), and Cs₂CO₃ (85 mg, 0.260 mmol) respectively and the vial was vacuumed and backfilled with N₂. Dioxane (0.8 mL) and water (0.2 mL) were added and the mixture was irradiated at 140° C. for 10 min. Diluted with DCM, washed with sat. NaHCO₃ once and brine once. Dried, filtered, concentrated, and purified by CombiFlash (Acetone/cyclohexane: 0˜40%) to give 2-(2-cyclopropyl-4-methoxyphenyl)-8-(oxazol-2-yl)-3-(oxazol-5-ylmethyl)benzo[4,5]thieno[2,3-d]pyrimidin-4(3H)-one (15.0 mg, 34.9% yield) as a white solid. LC-MS observed [M+H], 497.13. ¹H NMR (400 MHz, CDCl₃) δ 8.86 (dd, J=7.9, 1.2 Hz, 1H), 8.22 (dd, J=7.7, 1.2 Hz, 1H), 7.82 (d, J=0.8 Hz, 1H), 7.76 (s, 1H), 7.69 (t, J=7.8 Hz, 1H), 7.42 (d, J=0.8 Hz, 1H), 7.28 (d, J=8.3 Hz, 1H), 6.90-6.81 (m, 2H), 6.51 (d, J=2.5 Hz, 1H), 5.64 (d, J=15.4 Hz, 1H), 5.13 (d, J=15.3 Hz, 1H), 3.87 (s, 3H), 1.71-1.54 (m, 1H), 0.97-0.80 (m, 3H), 0.73-0.63 (m, 1H). 8-butyl-2-(2-cyclopropyl-4-methoxyphenyl)-3-(oxazol-5-ylmethyl)benzo[4,5]thieno[2,3-d]pyrimidin-4(3H)-one (4.4 mg, 10.4% yield) was obtained as well. LC-MS observed [M+H], 486.18. ¹H NMR (400 MHz, CDCl₃) δ 8.56 (dd, J=8.0, 1.2 Hz, 1H), 7.74 (s, 1H), 7.51 (t, J=7.7 Hz, 1H), 7.32 (dd, J=7.4, 1.2 Hz, 1H), 7.25 (d, J=8.4 Hz, 1H), 6.88-6.81 (m, 2H), 6.49 (d, J=2.5 Hz, 1H), 5.63 (d, J=15.4 Hz, 1H), 5.08 (d, J=15.3 Hz, 1H), 3.86 (s, 3H), 2.91 (t, J=7.7 Hz, 2H), 1.84-1.72 (m, 2H), 1.63-1.52 (m, 1H), 1.49-1.37 (m, 2H), 0.97 (t, J=7.2 Hz, 3H), 0.94-0.80 (m, 3H), 0.73-0.61 (m, 1H).

### Synthesis of (2-(2-cyclopropyl-4-methoxyphenyl)-3-(oxazol-5-ylmethyl)-4-oxo-3,4-dihydrobenzo[4,5]thieno[2,3-d]pyrimidin-8-yl)boronic acid

### Example 11

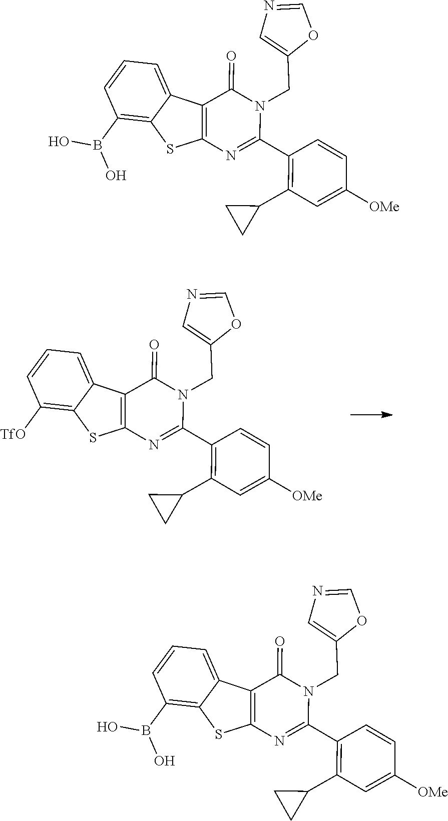 [SMILES: COc1ccc(-c2nc3sc4c(B(O)O)cccc4c3c(=O)n2Cc2cnco2)c(C2CC2)c1 ; COc1ccc(-c2nc3sc4c(OS(=O)(=O)C(F)(F)F)cccc4c3c(=O)n2Cc2cnco2)c(C2CC2)c1 ; COc1ccc(-c2nc3sc4c(B(O)O)cccc4c3c(=O)n2Cc2cnco2)c(C2CC2)c1]

To a 2 mL microwave vial were added 2-(2-cyclopropyl-4-methoxyphenyl)-3-(oxazol-5-ylmethyl)-4-oxo-3,4-dihydrobenzo[4,5]thieno[2,3-d]pyrimidin-8-yl trifluoromethanesulfonate (30.0 mg, 0.052 mmol), hypodiboric acid (14.0 mg, 0.156 mmol), potassium acetate (15.3 mg, 0.156 mmol), XPhos-Pd-G2 (2.0 mg, 2.60 μmol), XPhos (2.5 mg, 5.19 μmol), and EtOH (1.0 mL) respectively and the vial was irradiated via microwave at 130° C. for 15 min. After cooling to rt, the mixture was diluted with DCM and washed with Sat. NaHCO₃ and brine. Dried, filtered, concentrated, and purified by Prep TLC (7% MeOH/DCM) to give (2-(2-cyclopropyl-4-methoxyphenyl)-3-(oxazol-5-ylmethyl)-4-oxo-3,4-dihydrobenzo[4,5]thieno[2,3-d]pyrimidin-8-yl)boronic acid (1.5 mg, 6.1% yield) as a white foam. LC-MS observed [M+H], 474.13. ¹H NMR (400 MHz, CD₃OD/CDCl₃) δ 8.74 (d, J=8.0, 1H), 8.02-7.67 (m, 1H), 7.90 (s, 1H), 7.59 (t, J=8.0 Hz, 1H), 7.29 (d, J=8.4 Hz, 1H), 6.94-6.78 (m, 2H), 6.53 (d, J=2.1 Hz, 1H), 5.59 (d, J=15.5 Hz, 1H), 5.14 (d, J=15.3 Hz, 1H), 3.88 (s, 3H), 1.69-1.50 (m, 1H), 1.01-0.77 (m, 3H), 0.76-0.61 (m, 1H).

### Synthesis of 2-(2-cyclopropyl-4-methoxyphenyl)-3-(oxazol-5-ylmethyl)-4-oxo-3,4-dihydrobenzo[4,5]thieno[2,3-d]pyrimidine-8-sulfonic acid (example 12)

### Example 12

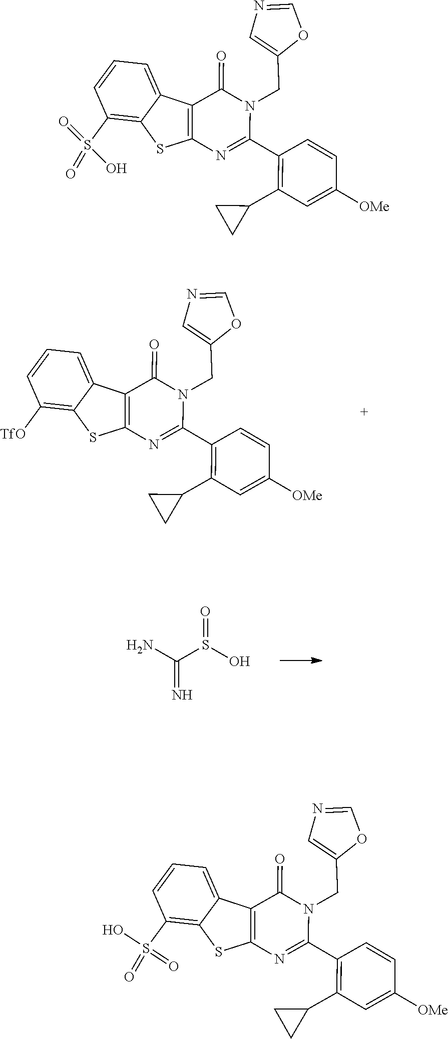 [SMILES: COc1ccc(-c2nc3sc4c(S(=O)(=O)O)cccc4c3c(=O)n2Cc2cnco2)c(C2CC2)c1 ; COc1ccc(-c2nc3sc4c(OS(=O)(=O)C(F)(F)F)cccc4c3c(=O)n2Cc2cnco2)c(C2CC2)c1 ; N=C(N)S(=O)O ; COc1ccc(-c2nc3sc4c(S(=O)(=O)O)cccc4c3c(=O)n2Cc2cnco2)c(C2CC2)c1]

To a 2 mL microwave vial were added 2-(2-cyclopropyl-4-methoxyphenyl)-3-(oxazol-5-ylmethyl)-4-oxo-3,4-dihydrobenzo[4,5]thieno[2,3-d]pyrimidin-8-yl trifluoromethanesulfonate (20 mg, 0.035 mmol), thiourea dioxide (11 mg, 0.10 mmol), PdCl₂(dppf) (5.1 mg, 0.069 mmol), cesium carbonate (23 mg, 0.069 mmol), and DMSO (1 mL) respectively under N₂ and the mixture was irradiated at 135° C. for 15 min under microwave. The crude mixture was diluted with MeOH (5 mL) and treated with 30% H₂O₂ (˜5 mL) for 4 h at rt. The mixture was purified by Prep HPLC (ACN/water: 20˜90%) to give 2-(2-cyclopropyl-4-methoxyphenyl)-3-(oxazol-5-ylmethyl)-4-oxo-3,4-dihydrobenzo[4,5]thieno[2,3-d]pyrimidine-8-sulfinic acid (5.1 mg, 29.0% yield) as a white solid. LC-MS observed [M+H], 510.08.

### Synthesis of 2-(2-cyclopropyl-4-methoxyphenyl)-8-fluoro-3-(oxazol-5-ylmethyl)benzo[4,5]thieno[2,3-d]pyrimidin-4(3H)-one (example 13)

### Example 13

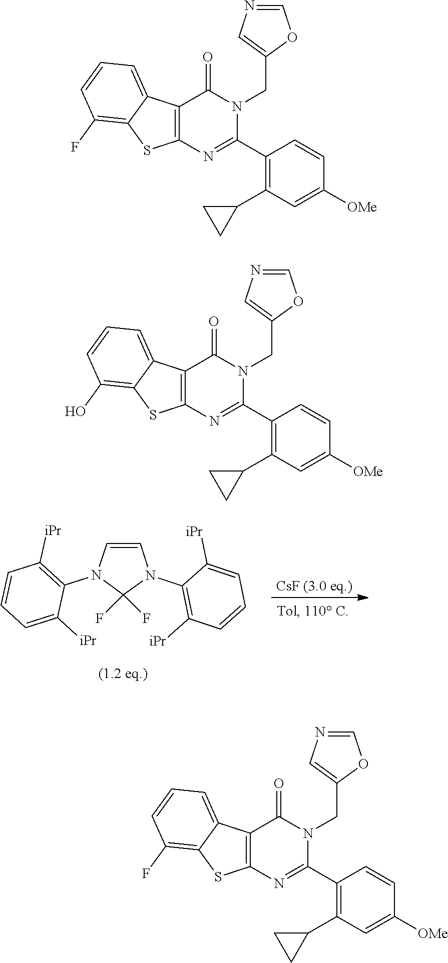

2-(2-cyclopropyl-4-methoxyphenyl)-8-hydroxy-3-(oxazol-5-ylmethyl)benzo[4,5]thieno[2,3-d]pyrimidin-4(3H)-one (0.05 g, 0.112 mmol, 1.0 eq.) and dried cesium fluoride (0.051 g, 0.337 mmol, 3 eq.) were placed in a vial. The vial was evacuated and dried at 110° C. for 2 hr for further drying. The vial was backfilled with nitrogen. A solution of 1,3-bis(2,6-diisopropylphenyl)-2,2-difluoro-2,3-dihydro-1H-imidazole (1.347 mL, 0.135 mmol, 1.2 eq.) in toluene was added via syringe. The mixture was stirred at 23° C. for 30 min, then at 110° C. for 24 h. Once cooled to 23° C., the mixture was filtered through a pad of Celite eluted with CH₂Cl₂ (3×3 mL). The filtrate was concentrated in vacuo and then purified by flash silica gel column chromatography. The desired fraction was purified further through prep HPLC again to afford 2-(2-cyclopropyl-4-methoxyphenyl)-8-fluoro-3-(oxazol-5-ylmethyl)benzo[4,5]thieno[2,3-d]pyrimidin-4(3H)-one (4.0 mg, 8%). LC-MS observed [M+H], 448.2. ¹H NMR (400 MHz, MeOD) δ 8.34 (d, J=8.0 Hz, 1H), 8.02 (d, J=2.0 Hz, flH), 7.54 (td, J=8.1, 5.2 Hz, 1H), 7.29-7.21 (m, 2H), 6.85 (dd, J=8.5, 2.5 Hz, 1H), 6.80 (s, 1H), 6.50 (d, J=2.4 Hz, 1H), 5.48 (d, J=15.8 Hz, 1H), 5.19 (d, J=15.7 Hz, 1H), 3.80 (d, J=0.8 Hz, 3H), 1.52 (ddd, J=13.3, 8.5, 5.0 Hz, 1H), 0.89-0.68 (m, 3H), 0.62 (dt, J=10.0, 5.1 Hz, 1H). ¹⁹F NMR (376 MHz, MeOD) δ −117.07.

### Synthesis of 2-(2-cyclopropyl-4-methoxyphenyl)-3-(oxazol-5-ylmethyl)-8-vinylbenzo[4,5]thieno[2,3-d]pyrimidin-4(3H)-one (example 14)

### Example 14

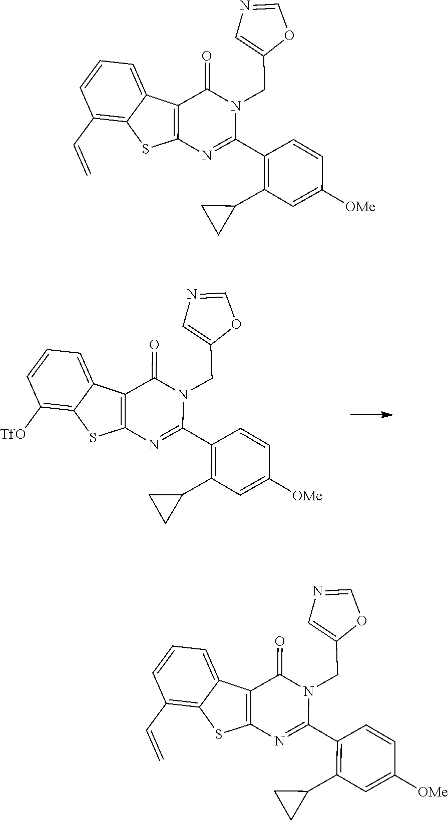 [SMILES: C=Cc1cccc2c1sc1nc(-c3ccc(OC)cc3C3CC3)n(Cc3cnco3)c(=O)c12 ; COc1ccc(-c2nc3sc4c(OS(=O)(=O)C(F)(F)F)cccc4c3c(=O)n2Cc2cnco2)c(C2CC2)c1 ; C=Cc1cccc2c1sc1nc(-c3ccc(OC)cc3C3CC3)n(Cc3cnco3)c(=O)c12]

To a 5 mL microwave vial were added 2-(2-cyclopropyl-4-methoxyphenyl)-3-(oxazol-5-ylmethyl)-4-oxo-3,4-dihydrobenzo[4,5]thieno[2,3-d]pyrimidin-8-yl trifluoromethanesulfonate (217 mg, 0.376 mmol), potassium vinyltrifluoroborate (151 mg, 1.127 mmol), Cs₂CO₃ (367 mg, 1.127 mmol), PdCl₂(dppf) (27.5 mg, 0.038 mmol), dioxane (3.01 mL), and water (0.751 mL) and the mixture was irradiated under microwave at 130° C. for 30 min. The reaction was quenched by sat. NH₄Cl solution, extracted by EtOAc, and then the organic layer dried (Na₂SO₄). Filtered, concentrated, and purified by combiflash (acetone/cyclohexane, 0-40%) to give 2-(2-cyclopropyl-4-methoxyphenyl)-3-(oxazol-5-ylmethyl)-8-vinylbenzo[4,5]thieno[2,3-d]pyrimidin-4(3H)-one as a white solid. LC-MS observed [M+H], 456.14. ¹H NMR (400 MHz, Acetone) δ 8.61 (dt, J=7.8, 1.3 Hz, 1H), 7.99 (s, 1H), 7.73 (dd, J=7.6, 1.4 Hz, 1H), 7.63 (td, J=7.7, 1.3 Hz, 1H), 7.42 (d, J=8.5 Hz, 1H), 7.05 (ddd, J=17.6, 11.2, 1.3 Hz, 1H), 6.92 (ddd, J=8.4, 2.5, 0.8 Hz, 1H), 6.87 (s, 1H), 6.58 (d, J=2.5 Hz, 1H), 6.07 (dd, J=17.5, 1.0 Hz, 1H), 5.65-5.54 (m, 2H), 5.23 (dd, J=15.8, 1.0 Hz, 1H), 3.89 (s, 3H), 1.84-1.71 (m, 1H), 0.98-0.82 (m, 3H), 0.77-0.66 (m, 1H).

### Synthesis of 2-(2-cyclopropyl-4-methoxyphenyl)-3-(oxazol-5-ylmethyl)-4-oxo-3,4-dihydrobenzo[4,5]thieno[2,3-d]pyrimidine-8-carbaldehyde (example 15)

### Example 15

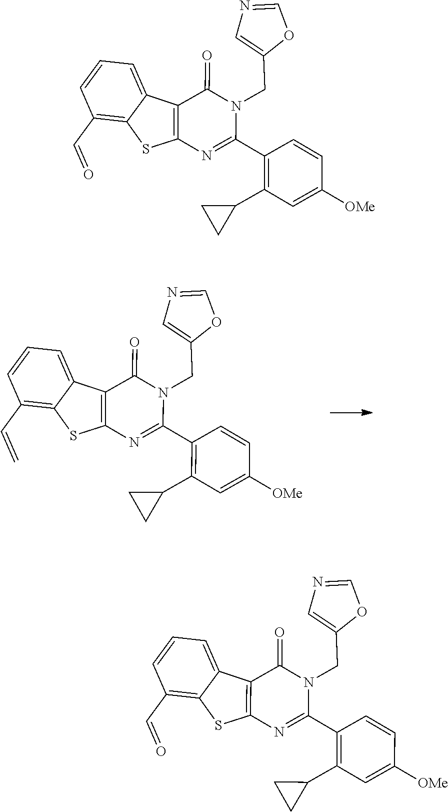 [SMILES: COc1ccc(-c2nc3sc4c(C=O)cccc4c3c(=O)n2Cc2cnco2)c(C2CC2)c1 ; C=Cc1cccc2c1sc1nc(-c3ccc(OC)cc3C3CC3)n(Cc3cnco3)c(=O)c12 ; COc1ccc(-c2nc3sc4c(C=O)cccc4c3c(=O)n2Cc2cnco2)c(C2CC2)c1]

A solution of osmium tetroxide in tert-butanol (0.033 mL, 2.5% wt, 0.00263 mmol) was added to a solution of 2-(2-cyclopropyl-4-methoxyphenyl)-3-(oxazol-5-ylmethyl)-8-vinylbenzo[4,5]thieno[2,3-d]pyrimidin-4(3H)-one (60 mg, 0.132 mmol) in dioxane (1.88 mL) and water (0.75 mL) and the mixture was stirred at rt for 15 minutes followed by the addition of sodium periodate (127 mg, 0.593 mmol). The slurry was stirred at rt for 6 hours. The reaction mixture was extracted with dichloromethane and the organic layer was washed with sat. NaHSO₃ solution. Dried, filtered, and concentrated to give 2-(2-cyclopropyl-4-methoxyphenyl)-3-(oxazol-5-ylmethyl)-4-oxo-3,4-dihydrobenzo[4,5]thieno[2,3-d]pyrimidine-8-carbaldehyde (68 mg, quan. yield) as a brown solid without further purification. LC-MS observed [M+H], 458.13. ¹H NMR (500 MHz, Acetone) δ 10.22 (s, 1H), 8.83 (dd, J=7.9, 1.3 Hz, 1H), 8.13 (dd, J=7.5, 1.2 Hz, 1H), 7.85 (s, 1H), 7.77 (t, J=7.6 Hz, 1H), 7.28 (d, J=8.5 Hz, 1H), 6.78 (dd, J=8.5, 2.5 Hz, 1H), 6.73 (s, 1H), 6.44 (d, J=2.5 Hz, 1H), 5.46 (dd, J=15.7, 0.9 Hz, 1H), 5.11 (dd, J=15.6, 1.0 Hz, 1H), 3.75 (s, 3H), 1.66-1.58 (m, 1H), 0.81-0.68 (m, 3H), 0.65-0.53 (m, 1H).

### Synthesis of 2-(2-cyclopropyl-4-methoxyphenyl)-8-(difluoromethyl)-3-(oxazol-5-ylmethyl)benzo[4,5]thieno[2,3-d]pyrimidin-4(3H)-one (example 16)

### Example 16

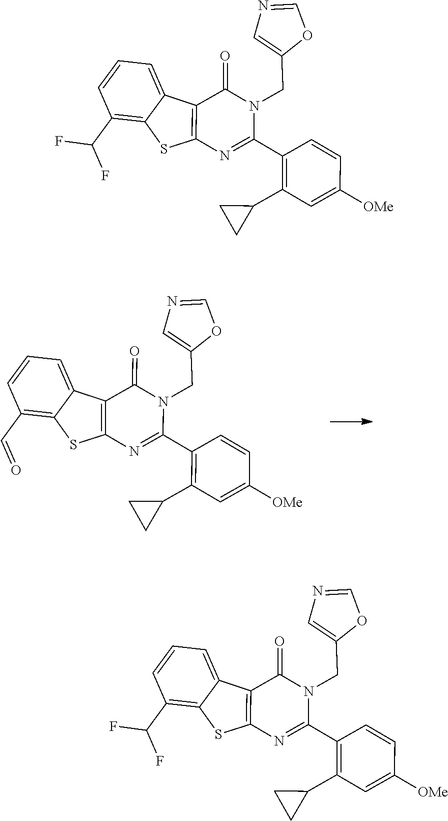 [SMILES: COc1ccc(-c2nc3sc4c(C(F)F)cccc4c3c(=O)n2Cc2cnco2)c(C2CC2)c1 ; COc1ccc(-c2nc3sc4c(C=O)cccc4c3c(=O)n2Cc2cnco2)c(C2CC2)c1 ; COc1ccc(-c2nc3sc4c(C(F)F)cccc4c3c(=O)n2Cc2cnco2)c(C2CC2)c1]

A mixture of 2-(2-cyclopropyl-4-methoxyphenyl)-3-(oxazol-5-ylmethyl)-4-oxo-3,4-dihydrobenzo[4,5]thieno[2,3-d]pyrimidine-8-carbaldehyde (43 mg, 0.094 mmol) and diethylaminosulfur trifluoride (0.124 mL, 0.94 mmol) was stirred at rt for 5 h. Diluted with dichloromethane (50 mL) and then quenched slowly with water (50 mL). The organic layer was washed with a saturated aqueous solution of sodium bicarbonate (twice) and brine. Dried, filtered, concentrated, and purified by Prep HPLC (ACN/water: 40˜90%) to give 2-(2-cyclopropyl-4-methoxyphenyl)-8-(difluoromethyl)-3-(oxazol-5-ylmethyl)benzo[4,5]thieno[2,3-d]pyrimidin-4(3H)-one (13 mg, 28.8% yield) as a white solid. LC-MS observed [M+H], 480.12.

### Synthesis of (2S,3S,4S,5R,6S)-6-((2-(2-cyclopropyl-4-methoxyphenyl)-3-(oxazol-5-ylmethyl)-4-oxo-3,4-dihydrobenzo[4,5]thieno[2,3-d]pyrimidin-8-yl)oxy)-3,4,5-trihydroxytetrahydro-2H-pyran-2-carboxylic acid (example 17)

### Example 17

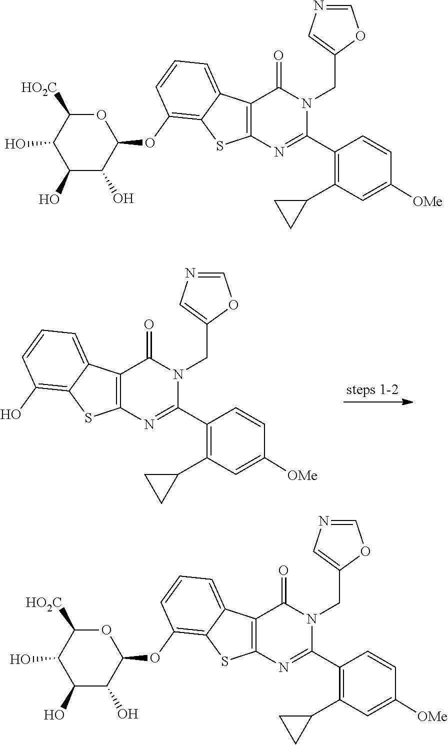 [SMILES: COc1ccc(-c2nc3sc4c(O[C@@H]5O[C@H](C(=O)O)[C@@H](O)[C@H](O)[C@H]5O)cccc4c3c(=O)n2Cc2cnco2)c(C2CC2)c1 ; COc1ccc(-c2nc3sc4c(O)cccc4c3c(=O)n2Cc2cnco2)c(C2CC2)c1 ; COc1ccc(-c2nc3sc4c(O[C@@H]5O[C@H](C(=O)O)[C@@H](O)[C@H](O)[C@H]5O)cccc4c3c(=O)n2Cc2cnco2)c(C2CC2)c1]

### Step 1

To a 2-dram vial were added 2-(2-cyclopropyl-4-methoxyphenyl)-8-hydroxy-3-(oxazol-5-ylmethyl)benzo[4,5]thieno[2,3-d]pyrimidin-4(3H)-one (100 mg, 0.224 mmol), (2R,3R,4S,5S,6S)-2-bromo-6-(methoxycarbonyl)tetrahydro-2H-pyran-3,4,5-triyl triacetate (267 mg, 0.673 mmol), silver carbonate on celite (619 mg, 1.122 mmol) and DCE (3.94 mL) respectively and the suspension was stirred at 60° C. for 10 h. Diluted with DCM, filtered via a pad of celite, concentrated, loaded on a cartridge, purified by Combiflash (SiO₂, Acetone/cyclohexane: 0-100%) to give (2S,3R,4S,5S,6S)-2-((2-(2-cyclopropyl-4-methoxyphenyl)-3-(oxazol-5-ylmethyl)-4-oxo-3,4-dihydrobenzo[4,5]thieno[2,3-d]pyrimidin-8-yl)oxy)-6-(methoxycarbonyl)tetrahydro-2H-pyran-3,4,5-triyl triacetate (8 mg, 4.7% yield) as a white foam. LC-MS observed [M+H], 762.20.

### Step 2

To a 1-dram vial containing (2S,3R,4S,5S,6S)-2-((2-(2-cyclopropyl-4-methoxyphenyl)-3-(oxazol-5-ylmethyl)-4-oxo-3,4-dihydrobenzo[4,5]thieno[2,3-d]pyrimidin-8-yl)oxy)-6-(methoxycarbonyl)tetrahydro-2H-pyran-3,4,5-triyl triacetate (8 mg, 10.50 μmol) were added THF (0.140 mL), Water (0.070 mL) and lithium hydroxide solution (105 μL, 0.105 mmol) respectively and the RXN was stirred at rt for 2.5 h. Quenched with acetic acid (6.01 μL, 0.105 mmol). Diluted with ACN, purified by Prep HPLC to give (2S,3S,4S,5R,6S)-6-((2-(2-cyclopropyl-4-methoxyphenyl)-3-(oxazol-5-ylmethyl)-4-oxo-3,4-dihydrobenzo[4,5]thieno[2,3-d]pyrimidin-8-yl)oxy)-3,4,5-trihydroxytetrahydro-2H-pyran-2-carboxylic acid (3.5 mg, 53.6%) as a white foam. LC-MS observed [M−H], 620.13. ¹H NMR (500 MHz, CD₃OD) δ 8.27 (d, J=7.9 Hz, 1H), 8.06 (s, 1H), 7.53 (t, J=8.0 Hz, 1H), 7.37 (d, J=8.0 Hz, 1H), 7.30 (dd, J=8.5, 7.1 Hz, 1H), 6.89 (dt, J=8.5, 2.8 Hz, 1H), 6.83 (s, 1H), 6.54 (d, J=2.5 Hz, 1H), 5.53 (dd, J=15.8, 4.0 Hz, 1H), 5.26-5.17 (m, 2H), 3.85 (s, 3H), 3.65-3.56 (m, 2H), 3.56-3.50 (m, 1H), 1.62-1.50 (m, 1H), 0.93-0.82 (m, 3H), 0.69-0.62 (m, 1H).

### Synthesis of 5,7-dichloro-2-(2-cyclopropyl-4-methoxyphenyl)-8-hydroxy-3-(oxazol-5-ylmethyl)benzo[4,5]thieno[2,3-d]pyrimidin-4(3H)-one (example 18)

### Example 18

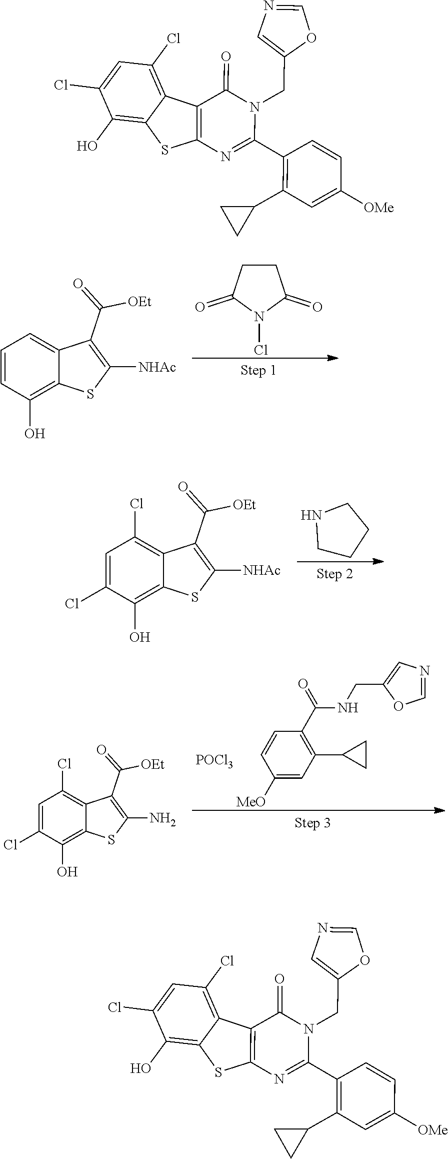

### Step 1

To a 40 mL vial were added ethyl 2-acetamido-7-hydroxybenzo[b]thiophene-3-carboxylate (300 mg, 1.074 mmol), NCS (430 mg, 3.22 mmol), and Acetonitrile (10.74 mL) and the solution was stirred at 70° C. for 3 h. Cooled to 0° C., quenched with 10 mL of Sat. Na₂S₂O₃ solution and the mixture was stirred at rt overnight. Diluted with DCM, washed with Sat. NaHCO₃ and dried (Na₂SO₄). Filtered, concentrated, and purified by CombiFlash (Acetone/cyclohexane: 0-100%) to give ethyl 2-acetamido-4,6-dichloro-7-hydroxybenzo[b]thiophene-3-carboxylate (137 mg, 36.6% yield) as an off-white solid. LC-MS observed [M−H], 345.97.

### Step 2

To a 2-dram vial were added ethyl 2-acetamido-4,6-dichloro-7-hydroxybenzo[b]thiophene-3-carboxylate (70 mg, 0.20 mmol), toluene (1.5 mL) and pyrrolidine (0.29 g, 0.33 mL, 4.0 mmol) and the solution was heated at 100° C. for 18 h. Diluted with DCM, washed with Sat. NaHCO₃ and brine. Dried, filtered, concentrated, and purified by CombiFlash (Acetone/cyclohexane: 0-100%) to give ethyl 2-amino-4,6-dichloro-7-hydroxybenzo[b]thiophene-3-carboxylate (28.8 mg, 47% yield). LC-MS observed [M−H], 303.96.

### Step 3

To a 2-dram vial were added JL-08016-003 (28.8 mg, 94.1 μmol), 2-cyclopropyl-4-methoxy-N-(oxazol-5-ylmethyl)benzamide (25.6 mg, 94.1 μmol), DCE (1 mL) followed by addition of phosphoryl trichloride (18.7 mg, 11.4 μL, 122 μmol). The suspension was stirred at 80° C. for 20 h. Cooled to rt. DCM and Sat. NaHCO₃ were added, and the mixture was stirred at rt for 1 h. The mixture was diluted with DCM, washed with Sat. NaHCO₃ and brine respectively. Dried, filtered, concentrated, and purified by Prep HPLC (ACN/water, 15˜90%, over 40 min) to give 5,7-dichloro-2-(2-cyclopropyl-4-methoxyphenyl)-8-hydroxy-3-(oxazol-5-ylmethyl)benzo[4,5]thieno[2,3-d]pyrimidin-4(3H)-one (10.4 mg, 21.5%) as a white solid. LC-MS observed [M−H], 514.04. ¹H NMR (400 MHz, DMSO) δ 11.06 (s, 1H), 8.21 (s, 1H), 7.65 (s, 1H), 7.37 (d, J=8.5 Hz, 1H), 6.91-6.83 (m, 1H), 6.86 (s, 1H), 6.47 (d, J=2.5 Hz, 1H), 5.35 (d, J=15.9 Hz, 1H), 5.03 (d, J=15.9 Hz, 1H), 3.80 (s, 3H), 1.67-1.56 (m, 1H), 1.23 (s, 1H), 0.87-0.79 (m, 3H), 0.68-0.59 (m, 1H).

### Synthesis of 5,7-dichloro-2-(2-cyclopropyl-4-methoxyphenyl)-8-hydroxy-3-(oxazol-5-ylmethyl)benzo[4,5]thieno[2,3-d]pyrimidin-4(3H)-one (example 19)

### Example 19

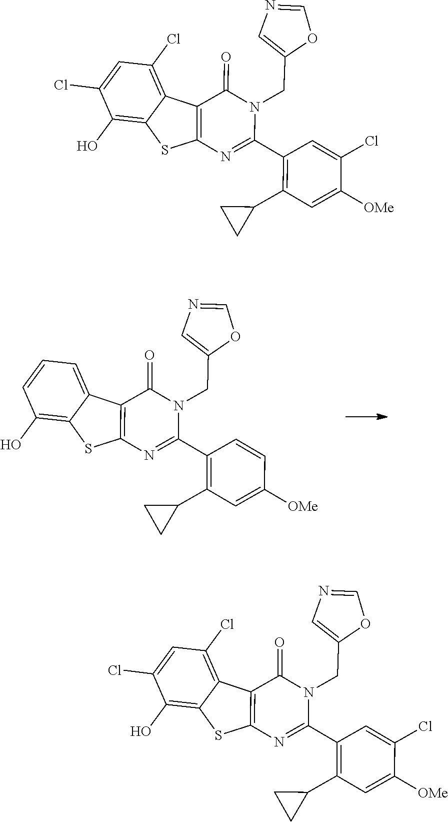 [SMILES: COc1cc(C2CC2)c(-c2nc3sc4c(O)c(Cl)cc(Cl)c4c3c(=O)n2Cc2cnco2)cc1Cl ; COc1ccc(-c2nc3sc4c(O)cccc4c3c(=O)n2Cc2cnco2)c(C2CC2)c1 ; COc1cc(C2CC2)c(-c2nc3sc4c(O)c(Cl)cc(Cl)c4c3c(=O)n2Cc2cnco2)cc1Cl]

To a 1-dram vial were added 2-(2-cyclopropyl-4-methoxyphenyl)-8-hydroxy-3-(oxazol-5-ylmethyl)benzo[4,5]thieno[2,3-d]pyrimidin-4(3H)-one (50 mg, 0.112 mmol), NCS (30.0 mg, 0.224 mmol), and Acetonitrile (1.122 mL) and the suspension was stirred at 70° C. for 0.5 h. The reaction mixture was absorbed on SiO₂ and purified by Prep TLC (acetone/cyclohexane: 40%) to give 5,7-dichloro-2-(5-chloro-2-cyclopropyl-4-methoxyphenyl)-8-hydroxy-3-(oxazol-5-ylmethyl)benzo[4,5]thieno[2,3-d]pyrimidin-4(3H)-one (0.6 mg, 0.97% yield) as a white solid. LC-MS observed [M+H], 548.00. ¹H NMR (400 MHz, CDCl₃) δ 7.75 (s, 1H), 7.56 (s, 1H), 7.36 (s, 1H), 6.98 (s, 1H), 6.49 (s, 1H), 6.00 (s, 1H), 5.61-5.50 (m, 1H), 5.30 (s, 2H), 5.04 (t, J=15.5 Hz, 1H), 3.95 (s, 3H), 1.67-1.57 (m, 1H), 1.43 (s, 7H), 1.25 (s, 2H), 1.00-0.90 (m, 2H), 0.84 (dt, J=10.1, 5.1 Hz, 1H), 0.72-0.63 (m, 1H).

### Synthesis of 2-(2-cyclopropyl-4-methoxyphenyl)-8-(2-hydroxyethoxy)-3-(oxazol-5-ylmethyl)benzo[4,5]thieno[2,3-d]pyrimidin-4(3H)-one (example 20)

### Example 20

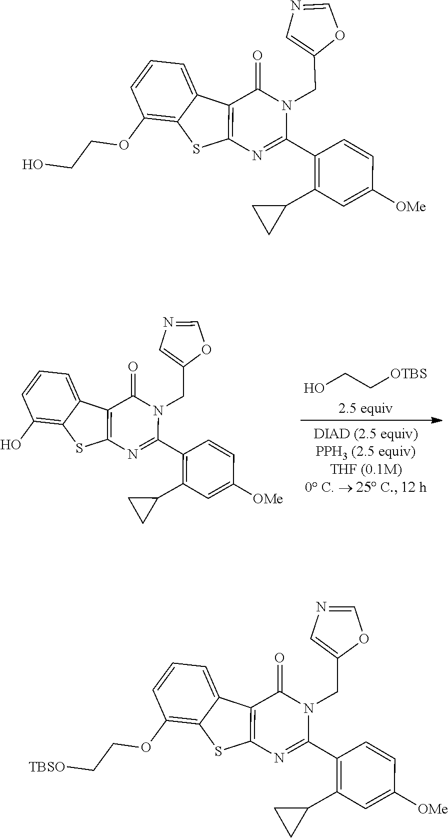 [SMILES: COc1ccc(-c2nc3sc4c(OCCO)cccc4c3c(=O)n2Cc2cnco2)c(C2CC2)c1 ; COc1ccc(-c2nc3sc4c(O)cccc4c3c(=O)n2Cc2cnco2)c(C2CC2)c1 ; CC(C)(C)[Si](C)(C)OCCO ; COc1ccc(-c2nc3sc4c(OCCO[Si](C)(C)C(C)(C)C)cccc4c3c(=O)n2Cc2cnco2)c(C2CC2)c1]

### Step 1

In a 4 mL vial equipped with a stir bar, 2-(2-cyclopropyl-4-methoxyphenyl)-8-hydroxy-3-(oxazol-5-ylmethyl)benzo[4,5]thieno[2,3-d]pyrimidin-4(3H)-one (100 mg, 1.0 equiv.), triphenylphosphine (147 mg, 2.5 equiv.) were dissolved in dry THF (2.2 mL) under nitrogen, followed by addition of 2-((tert-butyldimethylsilyl)oxy)ethan-1-ol (99 mg, 2.5 equiv.). The reaction mixture was cooled to 0° C. and added DIAD (109 μl, 2.5 equiv.). The ice bath was removed, and the reaction mixture was stirred at 25° C. for 12 h. Reaction progress was monitored through LC-MS. After completion, the reaction mixture was concentrated under vacuum and crude residue was re-dissolved in 3.0 mL of dimethyl sulfoxide, and purified through reversed phase combiflash to afford 8-(2-((tert-butyldimethylsilyl)oxy)ethoxy)-2-(2-cyclopropyl-4-methoxyphenyl)-3-(oxazol-5-ylmethyl)benzo[4,5]thieno[2,3-d]pyrimidin-4(3H)-one (83 mg, 61% yield). ESI MS m/z=604.5 [M+H]⁺.

### Step 2

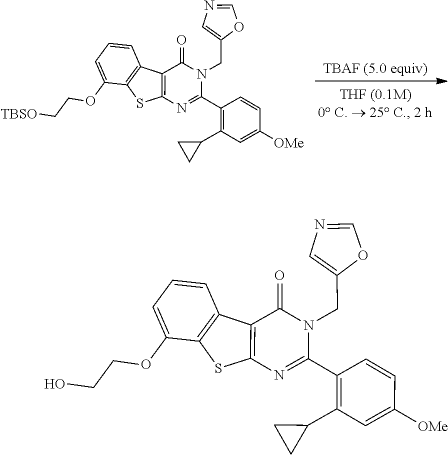 [SMILES: COc1ccc(-c2nc3sc4c(OCCO[Si](C)(C)C(C)(C)C)cccc4c3c(=O)n2Cc2cnco2)c(C2CC2)c1 ; COc1ccc(-c2nc3sc4c(OCCO)cccc4c3c(=O)n2Cc2cnco2)c(C2CC2)c1]

In a 4 mL vial equipped with a stir bar, 8-(2-((tert-butyldimethylsilyl)oxy)ethoxy)-2-(2-cyclopropyl-4-methoxyphenyl)-3-(oxazol-5-ylmethyl)benzo[4,5]thieno[2,3-d]pyrimidin-4(3H)-one (60.0 mg, 1.0 equiv.) was dissolved in dry THF (1 mL), followed by addition of TBAF (1.0 M in THF) (497 μl, 5.0 equiv.) dropwise at 0° C. Ice bath was removed and reaction mixture was stirred for 2 h at 25° C. Reaction mixture was concentrated under vacuum and crude residue was re-dissolved in 2.0 mL of dimethyl sulfoxide, passed through a 0.45 μm syringe filter, and purified by RPHPLC to afford 2-(2-cyclopropyl-4-methoxyphenyl)-8-(2-hydroxyethoxy)-3-(oxazol-5-ylmethyl)benzo[4,5]thieno[2,3-d]pyrimidin-4(3H)-one (23.0 mg, 47% yield). ESI MS m/z=490.2 [M+H]⁺. ¹H NMR (500 MHz, DMSO-d₆) δ 8.21 (s, 1H), 8.12 (d, J=7.9 Hz, 1H), 7.55 (t, J=8.0 Hz, 1H), 7.37 (d, J=8.4 Hz, 1H), 7.18 (d, J=8.1 Hz, 1H), 6.87 (dd, J=8.5, 2.5 Hz, 1H), 6.84 (s, 1H), 6.47 (d, J=2.5 Hz, 1H), 5.40 (d, J=15.9 Hz, 1H), 5.09 (d, J=15.9 Hz, 1H), 4.99 (t, J=5.4 Hz, 1H), 4.25 (t, J=4.9 Hz, 2H), 3.84-3.78 (m, 2H), 3.80 (s, 3H), 1.56 (ddd, J=13.2, 7.8, 5.6 Hz, 1H), 0.87-0.74 (m, 3H), 0.65-0.58 (m, 1H).

### Synthesis of 7-allyl-2-(2-cyclopropyl-4-methoxyphenyl)-8-hydroxy-3-(oxazol-5-ylmethyl)benzo[4,5]thieno[2,3-d]pyrimidin-4(3H)-one (example 21)

### Example 21

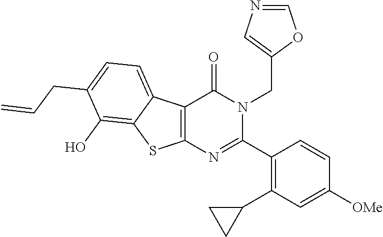 [SMILES: C=CCc1ccc2c(sc3nc(-c4ccc(OC)cc4C4CC4)n(Cc4cnco4)c(=O)c32)c1O]

### Step 1

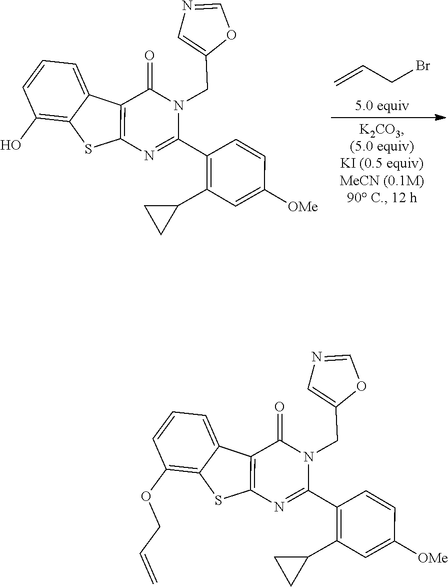

In a 20 mL vial equipped with stir bar, 2-(2-cyclopropyl-4-methoxyphenyl)-8-hydroxy-3-(oxazol-5-ylmethyl)benzo[4,5]thieno[2,3-d]pyrimidin-4(3H)-one (200 mg, 0.112 mmol), potassium carbonate (310 mg, 5.0 equiv.) and potassium iodide (3.73 mg, 0.05 equiv.) were combined neat under nitrogen atmosphere, followed by addition of dry acetonitrile (4.5 mL) and 3-bromoprop-1-ene (19.40 μl, 0.224 mmol). Reaction mixture was heated at 90° C. for 12 h in a heating block. Reaction progress was monitored through LC-MS. Reaction mixture was concentrated under vacuum and crude residue was re-dissolved in 4.0 mL of dimethyl sulfoxide and purified through reversed phase combiflash to afford 8-(allyloxy)-2-(2-cyclopropyl-4-methoxyphenyl)-3-(oxazol-5-ylmethyl)benzo[4,5]thieno[2,3-d]pyrimidin-4(3H)-one (165 mg, 76% yield). ESI MS m/z=486.2 [M+H]⁺. ¹H NMR (400 MHz, MeOD-d₄) δ 8.19 (dd, J=8.0, 0.9 Hz, 1H), 8.07 (s, 1H), 7.51 (t, J=8.0 Hz, 1H), 7.31 (d, J=8.5 Hz, 1H), 7.10 (d, J=8.0 Hz, 1H), 6.90 (dd, J=8.5, 2.5 Hz, 1H), 6.85 (s, 1H), 6.55 (d, J=2.5 Hz, 1H), 6.15 (ddt, J=17.2, 10.5, 5.2 Hz, 1H), 5.58-5.44 (m, 2H), 5.32 (dq, J=10.6, 1.5 Hz, 1H), 5.22 (dd, J=15.8, 1.0 Hz, 1H), 4.80 (dt, J=5.2, 1.6 Hz, 2H), 3.86 (s, 3H), 1.63-1.52 (m, 1H), 0.96-0.75 (m, 3H), 0.72-0.62 (m, 1H).

### Step 2

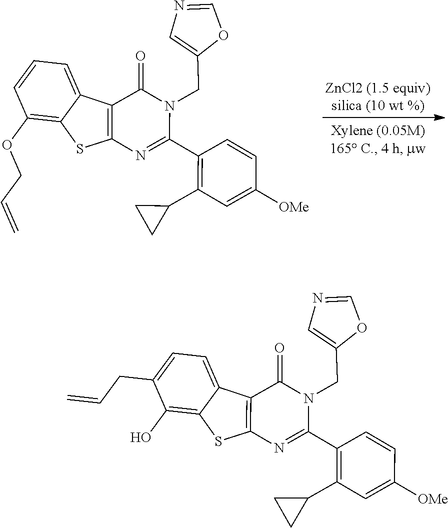 [SMILES: C=CCOc1cccc2c1sc1nc(-c3ccc(OC)cc3C3CC3)n(Cc3cnco3)c(=O)c12 ; C=CCc1ccc2c(sc3nc(-c4ccc(OC)cc4C4CC4)n(Cc4cnco4)c(=O)c32)c1O]

In a 25 mL microwave reaction vial equipped with a stir bar, 8-(allyloxy)-2-(2-cyclopropyl-4-methoxyphenyl)-3-(oxazol-5-ylmethyl)benzo[4,5]thieno[2,3-d]pyrimidin-4(3H)-one (190 mg, 0.391 mmol), zinc chloride (80 mg, 0.587 mmol) and Silica gel (19 mg, 10 wt %) were combined neat and followed by addition of xylene (7.83 ml). Then, the vial was placed under microwave reactor at 160° C. for 4 h. The reaction mixture was concentrated under vacuum and the crude residue was re-dissolved in 4.0 mL of dimethyl sulfoxide and purified through reversed phase combiflash to afford 7-allyl-2-(2-cyclopropyl-4-methoxyphenyl)-8-hydroxy-3-(oxazol-5-ylmethyl)benzo[4,5]thieno[2,3-d]pyrimidin-4(3H)-one (75 mg, 40% yield). ESI MS m/z=486.3 [M+H]⁺. ¹H NMR (500 MHz, DMSO-d₆) δ 9.80 (br, 1H), 8.21 (s, 1H), 7.99 (d, J=8.1 Hz, 1H), 7.36 (d, J=8.4 Hz, 1H), 7.31 (d, J=8.0 Hz, 1H), 6.87 (dd, J=8.5, 2.4 Hz, 1H), 6.83 (s, 1H), 6.47 (d, J=2.5 Hz, 1H), 6.06-5.95 (m, 1H), 5.39 (d, J=16.0 Hz, 1H), 5.11-5.02 (m, 3H), 3.81 (s, 3H), 3.53 (d, J=6.5 Hz, 2H), 1.56 (ddd, J=13.2, 7.9, 5.6 Hz, 1H), 0.87-0.77 (m, 3H), 0.67-0.58 (m, 1H).

### Synthesis of 2-(2-cyclopropyl-4-methoxyphenyl)-8-hydroxy-3-(oxazol-5-ylmethyl)-7-propylbenzo[4,5]thieno[2,3-d]pyrimidin-4(3H)-one (example 22)

### Example 22

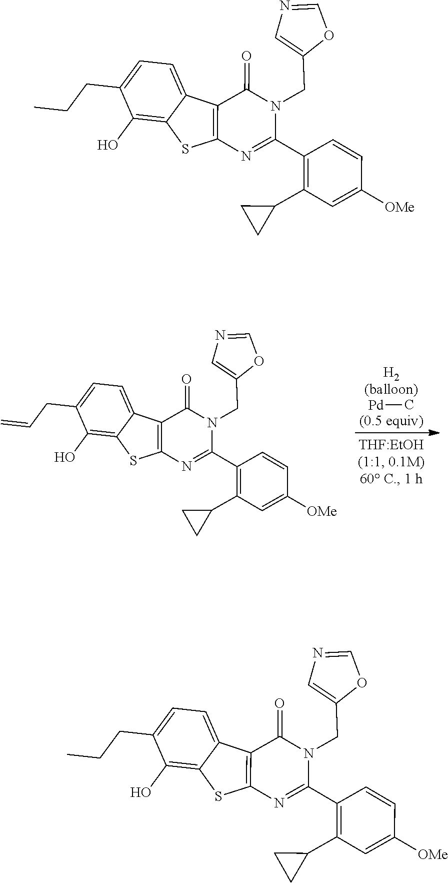

In a 4 mL vial equipped with stir bar, 7-allyl-2-(2-cyclopropyl-4-methoxyphenyl)-8-hydroxy-3-(oxazol-5-ylmethyl)benzo[4,5]thieno[2,3-d]pyrimidin-4(3H)-one (15 mg, 0.031 mmol) and 5 wt % Pd/C (32.9 mg, 0.5 equiv.) were combined neat, followed by addition of EtOH (0.309 ml) and THF (0.309 ml). Reaction mixtures were stirred and air-changed with a hydrogen balloon at room temperature, then heated to 60° C. for 1 h with a hydrogen balloon. The crude mixture was concentrated under vacuum and the residue was re-dissolved in 2.0 mL of dimethyl sulfoxide, passed through a 0.45 μm syringe filter, and purified by RPHPLC to afford 2-(2-cyclopropyl-4-methoxyphenyl)-8-hydroxy-3-(oxazol-5-ylmethyl)-7-propylbenzo[4,5]thieno[2,3-d]pyrimidin-4(3H)-one (8 mg, 53%). ESI MS m/z=488.2 [M+H]⁺. ¹H NMR (500 MHz, DMSO-d₆) δ 9.68 (br, 1H), 8.21 (s, 1H), 7.97 (d, J=8.0 Hz, 1H), 7.34 (dd, J=16.1, 8.3 Hz, 2H), 6.87 (dd, J=8.5, 2.5 Hz, 1H), 6.83 (s, 1H), 6.47 (d, J=2.5 Hz, 1H), 5.39 (d, J=15.9 Hz, 1H), 5.07 (d, J=15.9 Hz, 1H), 3.81 (s, 3H), 2.76-2.70 (m, 2H), 1.66-1.51 (m, 3H), 0.93 (t, J=7.3 Hz, 3H), 0.87-0.77 (m, 3H), 0.65-0.57 (m, 1H).

### Synthesis of 2-(2-cyclopropyl-4-methoxyphenyl)-3-(oxazol-5-ylmethyl)-4-oxo-3,4-dihydrobenzo[4,5]thieno[2,3-d]pyrimidin-8-yl hydrogen sulfate (example 23)

### Example 23

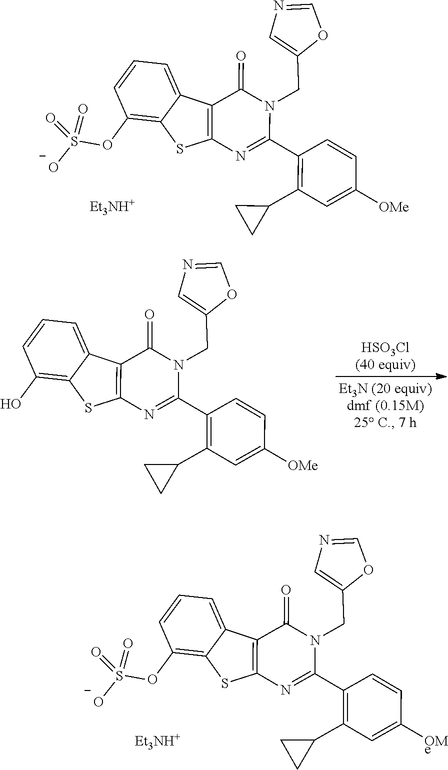 [SMILES: "Et3NH+" ; COc1ccc(-c2nc3sc4c(OS(=O)(=O)[O-])cccc4c3c(=O)n2Cc2cnco2)c(C2CC2)c1 ; "Et3NH+" ; COc1ccc(-c2nc3sc4c(O)cccc4c3c(=O)n2Cc2cnco2)c(C2CC2)c1 ; COc1ccc(-c2nc3sc4c(OS(=O)(=O)[O-])cccc4c3c(=O)n2Cc2cnco2)c(C2CC2)c1]

In a 20 mL reaction vial with a stir bar, 2-(2-cyclopropyl-4-methoxyphenyl)-8-hydroxy-3-(oxazol-5-ylmethyl)benzo[4,5]thieno[2,3-d]pyrimidin-4(3H)-one (150 mg, 1.0 equiv.) was dissolved in dry DMF (2.2 mL) under nitrogen. The reaction vial was placed at −10° C. dry ice bath and chlorosulfonic acid (451 μl, 20.0 equiv.) was added dropwise. After complete addition, the reaction vial was stirred for 4 h at 25° C. The reaction progress was monitored through LC-MS. Incomplete conversion of starting material was observed. 20 equiv. of chlorosulfonic acid (451 μl) was added and stirred for additional 3 h. The reaction mixture was directly loaded into reversed phase combiflash column and purified with acetonitrile/water eluent to afford 2-(2-cyclopropyl-4-methoxyphenyl)-3-(oxazol-5-ylmethyl)-4-oxo-3,4-dihydrobenzo[4,5]thieno [2,3-d]pyrimidin-8-yl hydrogen sulfate as a 1:1 triethylamine salt (54 mg, 30% yield). ESI MS m/z=526.2 [M+H]⁺. ¹H NMR (500 MHz, DMSO-d₆) δ 8.83 (s, 1H), 8.22 (dd, J=7.6, 1.4 Hz, 1H), 8.21 (s, 1H), 7.60-7.50 (m, 2H), 7.38 (d, J=8.5 Hz, 1H), 6.87 (dd, J=8.5, 2.5 Hz, 1H), 6.84 (s, 1H), 6.47 (d, J=2.5 Hz, 1H), 5.40 (d, J=16.0 Hz, 1H), 5.09 (d, J=16.0 Hz, 1H), 3.81 (s, 3H), 3.10 (qd, J=7.3, 4.8 Hz, 6H), 1.58 (tt, J=7.9, 5.5 Hz, 1H), 1.17 (t, J=7.3 Hz, 9H), 0.87-0.76 (m, 3H), 0.65-0.56 (m, 1H).

### Synthesis of 7-bromo-2-(2-cyclopropyl-4-methoxyphenyl)-8-hydroxy-3-(oxazol-5-ylmethyl)benzo[4,5]thieno[2,3-d]pyrimidin-4(3H)-one (example 24)

### Example 24

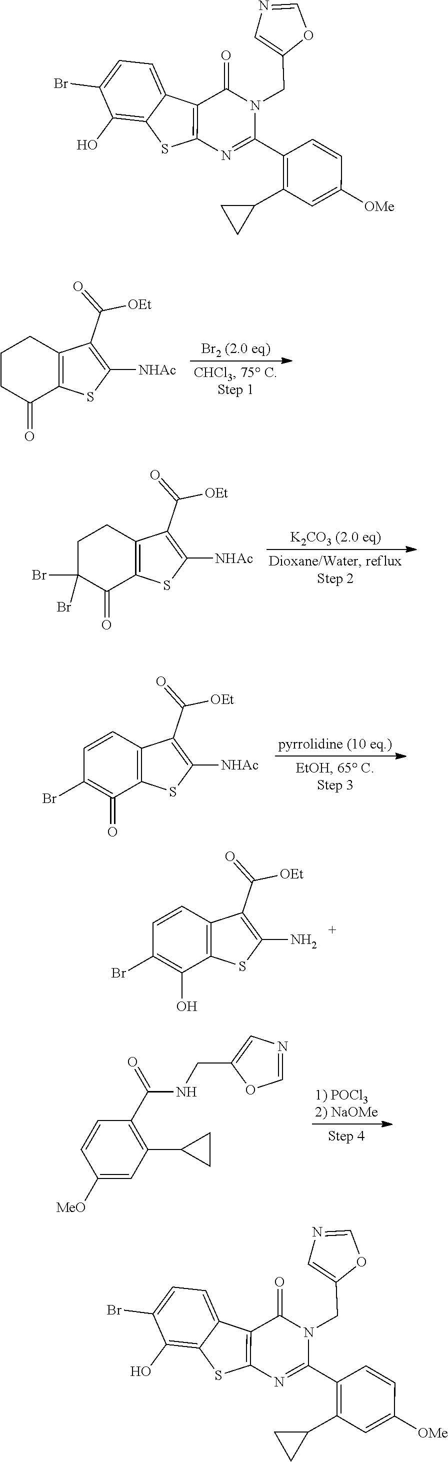

### Step 1

A solution of bromine (5.79 mL, 112 mmol, 2.0 eq.) in chloroform was slowly added to the solution of ethyl 2-acetamido-7-oxo-4,5,6,7-tetrahydrobenzo[b]thiophene-3-carboxylate (15.8 g, 56.2 mmol, 1.0 eq.) in CHCl₃. The mixture was heated to 75° C. for 3 h then cooled to rt. After overnight, the LCMS monitoring showed that the mono brominated product. Additional 1 mL of bromine (0.3 eq.) in 5 mL of CHCl₃ was added. The reaction was heated to 45° C. Upon completion, the reaction was quenched with the addition of sat. Na₂S₂O₃ solution until the brown color disappears. The reaction mixture was filtered through a pad of celite. Then the solution was extracted with DCM twice. The combined organic layers were washed with brine. Dried over Na₂SO₄, filtered, and concentrated. The crude mixture was suspended in 50 mL of MTBE overnight to afford 20 g of crude product by filtration. The crude was slurried in 2.5V of MTBE overnight and filtered. 17.6 g was collected and used for the next step. LC-MS observed [M+H], 439.9. ¹H NMR (400 MHz, DMSO) δ 11.33 (s, 1H), 7.11 (d, J=8.4 Hz, 1H), 6.70 (d, J=8.4 Hz, 1H), 4.35 (q, J=7.0 Hz, 2H), 2.24 (s, 3H), 1.37 (t, J=7.1 Hz, 3H).

### Step 2

A solution of K₂CO₃ (11.08 g, 80 mmol) in water (20.00 ml) was added to a solution of the crude of ethyl 2-acetamido-6,6-dibromo-7-oxo-4,5,6,7-tetrahydrobenzo[b]thiophene-3-carboxylate (17.6 g) in dioxane (120 ml) and water (25 mL). The mixture was heated to reflux. After reaction was complete, the mixture was cooled to r.t. Additional 20 mL of water was added and the mixture was stirred for overnight. The mixture was filtered and ethyl 2-acetamido-6-bromo-7-hydroxybenzo[b]thiophene-3-carboxylate (11.2 g, 58%, 2 steps) was obtained. LC-MS observed [M+H], 358.0, 360.0.

### Step 3

To a 250 mL round-bottomed flask were added ethyl 2-acetamido-6-bromo-7-hydroxybenzo[b]thiophene-3-carboxylate (4.97 g, 13.87 mmol, 1.0 eq.), pyrrolidine (11.47 mL, 139 mmol, 10 eq.) and EtOH (100 mL) respectively and the mixture was stirred at 65° C. until LCMS showed no SM. The mixture was quenched with sat. NH₄Cl. Then the aq. layer was extracted with EA and DCM. The combined organic layers were dried, filtered, and concentrated. The crude product was purified through column affording ethyl 2-amino-6-bromo-7-hydroxybenzo[b]thiophene-3-carboxylate (2.55 g, 58.1% yield) as a pink solid. LC-MS observed [M+H], 315.9, 317.9. ¹H NMR (400 MHz, MeOD) δ 7.50 (dd, J=8.7, 1.1 Hz, 1H), 7.31 (d, J=8.6 Hz, 1H), 4.60 (s, 1H, —NH₂), 4.38 (q, J=7.1 Hz, 2H), 1.43 (t, J=7.1 Hz, 3H).

### Step 4

To a vial were added 2-cyclopropyl-4-methoxy-N-(oxazol-5-ylmethyl)benzamide (1.189 g, 4.36 mmol, 1.0 eq.), ethyl 2-amino-6-bromo-7-hydroxybenzo[b]thiophene-3-carboxylate (1.38 g, 4.36 mmol, 1.0 eq.), DCE (25.7 mL) and POCl₃ (0.610 ml, 6.55 mmol, 1.5 eq.) respectively and the mixture was stirred at 80° C. for 4 h. The mixture was concentrated and dissolved in MeOH (20 mL). A solution of sodium methoxide in MeOH (4.99 mL, 21.82 mmol, 5.0 eq.) was added and the mixture was heated at 65° C. overnight. The mixture was concentrated and diluted with EtOAc and then washed with sat. NH₄Cl and brine. The organic layer was dried, filtered, concentrated, and purified through column chromatography affording 7-bromo-2-(2-cyclopropyl-4-methoxyphenyl)-8-hydroxy-3-(oxazol-5-ylmethyl)benzo[4,5]thieno[2,3-d]pyrimidin-4(3H)-one (1.1 g, 48%) as an ivory solid. LC-MS observed [M+H], 524.0, 526.0. ¹H NMR (400 MHz, MeOD) δ 8.09 (s, 1H), 8.04 (dd, J=8.5, 1.2 Hz, 1H), 7.67 (dd, J=8.5, 1.4 Hz, 1H), 7.33 (d, J=8.5 Hz, 1H), 6.92 (dd, J=8.5, 2.5 Hz, 1H), 6.86 (s, 1H), 6.57 (d, J=2.5 Hz, 1H), 5.54 (d, J=15.7 Hz, 1H), 5.24 (d, J=15.8 Hz, 1H), 3.87 (s, 3H), 1.59 (td, J=8.4, 4.3 Hz, 1H), 0.97-0.77 (m, 3H), 0.69 (dd, J=8.6, 3.7 Hz, 1H).

### Synthesis of 2-(2-cyclopropyl-4-methoxyphenyl)-3-((4-fluorooxazol-5-yl)methyl)-8-hydroxybenzo[4,5]thieno[2,3-d]pyrimidin-4(3H)-one (example 25)

### Example 25

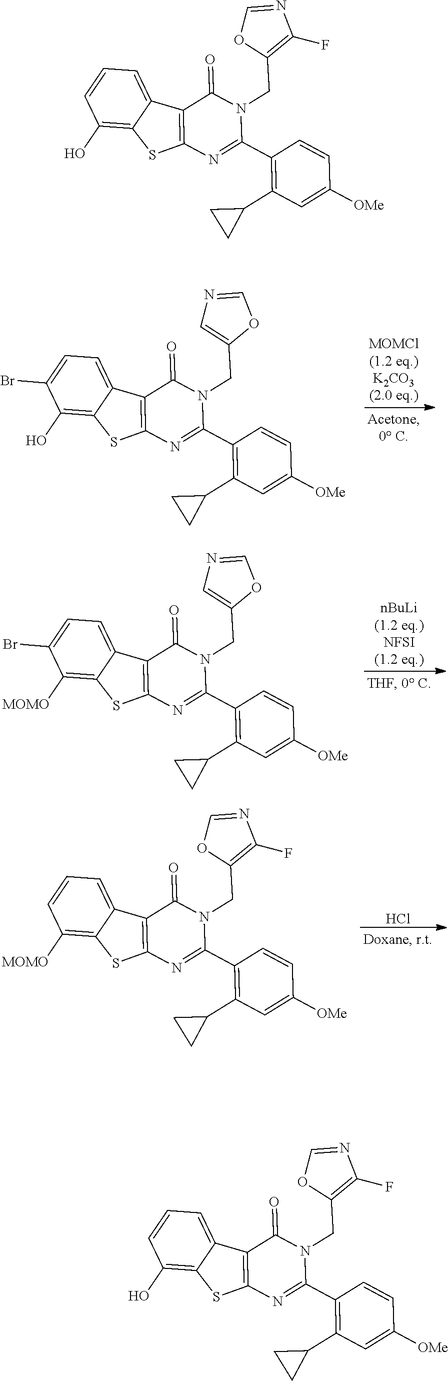

### Step 1

To the suspension of 7-bromo-2-(2-cyclopropyl-4-methoxyphenyl)-8-hydroxy-3-(oxazol-5-ylmethyl)benzo[4,5]thieno[2,3-d]pyrimidin-4(3H)-one (700 mg, 1.335 mmol, 1.0 eq.) and K₂CO₃ (369 mg, 2.67 mmol, 2.0 eq.) in acetone (0.2 M), was slowly added MOM-Cl (122 μL, 1.602 mmol, 1.2 eq.) at 0° C. The reaction mixture was slowly warmed to r.t. The reaction progress was monitored with LCMS. Upon completion, the mixture was concentrated and diluted with ethyl acetate. The organic layer was washed with water, sat. NaHCO₃(aq.) and brine respectively. Dried (Na₂SO₄), filtered, concentrated, and purified by CombiFlash to afford 7-bromo-2-(2-cyclopropyl-4-methoxyphenyl)-8-(methoxymethoxy)-3-(oxazol-5-ylmethyl)benzo[4,5]thieno[2,3-d]pyrimidin-4(3H)-one (690 mg, 91% yield) as a yellow solid. LC-MS observed [M+H], 568.0, 570.0. ¹H NMR (400 MHz, CDCl₃) δ 8.35 (d, J=8.6 Hz, 1H), 7.78-7.73 (m, 2H), 7.26 (d, J=8.5 Hz, 1H), 6.90-6.80 (m, 2H), 6.51 (d, J=2.5 Hz, 1H), 5.63 (d, J=15.3 Hz, 1H), 5.34 (s, 2H), 5.10 (d, J=15.4 Hz, 1H), 3.88 (s, 3H), 3.74 (s, 3H), 1.58 (s, 1H), 1.01-0.83 (m, 3H), 0.69 (q, J=4.8 Hz, 1H).

### Step 2

To a solution of 7-bromo-2-(2-cyclopropyl-4-methoxyphenyl)-8-(methoxymethoxy)-3-(oxazol-5-ylmethyl)benzo[4,5]thieno[2,3-d]pyrimidin-4(3H)-one (40.0 mg, 0.070 mmol) in THF (1 mL) was added BuLi (33.8 μL, 0.084 mmol, 1.2 eq., 2.5 M) at 0° C. The mixture was stirred at 0° C. for 1.5 h. A solution of N-fluorobenzenesulfonimide (26.6 mg, 0.084 mmol) in THF (0.5 mL) was added slowly. The reaction was monitored with LCMS. Upon completion, the reaction was diluted with EtOAc. The organic layer was washed with sat. NaHCO₃(aq.) and brine. The organic layer was dried over Na₂SO₄, filtered, concentrated, and purified with prep-HPLC to afford 2-(2-cyclopropyl-4-methoxyphenyl)-3-((4-fluorooxazol-5-yl)methyl)-8-(methoxymethoxy)benzo[4,5]thieno[2,3-d]pyrimidin-4(3H)-one. LC-MS observed [M+H], 508.1.

### Step 3

To a vial with 2-(2-cyclopropyl-4-methoxyphenyl)-3-((4-fluorooxazol-5-yl)methyl)-8-(methoxymethoxy)benzo[4,5]thieno[2,3-d]pyrimidin-4(3H)-one (2.0 mg, 3.9 μmol) was added 0.5 mL of dioxane. Then, 0.5 mL of 4M HCl in dioxane was added to the mixture. The reaction was stirred at 20° C. for 2 h and the mixture was concentrated. The crude product was purified through Prep-HPLC affording 2-(2-cyclopropyl-4-methoxyphenyl)-3-((4-fluorooxazol-5-yl)methyl)-8-hydroxybenzo[4,5]thieno[2,3-d]pyrimidin-4(3H)-one (0.5 mg, 30% yield). LC-MS observed [M+H], 464.1. ¹H NMR (400 MHz, CD₃OD) δ 8.57 (s, 1H), 8.11 (dd, J=7.9, 0.9 Hz, 1H), 7.86 (d, J=2.0 Hz, 1H), 7.42 (t, J=7.9 Hz, 1H), 7.34 (d, J=8.5 Hz, 1H), 6.94 (ddd, J=8.6, 5.0, 1.7 Hz, 2H), 6.57 (d, J=2.5 Hz, 1H), 5.52 (dd, J=15.9, 1.1 Hz, 1H), 5.24 (dd, J=15.9, 1.7 Hz, 1H), 3.87 (s, 3H), 1.59 (ddd, J=13.3, 8.4, 5.0 Hz, 1H), 0.99-0.77 (m, 3H), 0.76-0.61 (m, 1H). ¹⁹F NMR (376 MHz, MeOD) δ −145.37.

### Synthesis of 7-chloro-2-(2-cyclopropyl-4-methoxyphenyl)-8-hydroxy-3-(oxazol-5-ylmethyl)benzo[4,5]thieno[2,3-d]pyrimidin-4(3H)-one

### Example 26

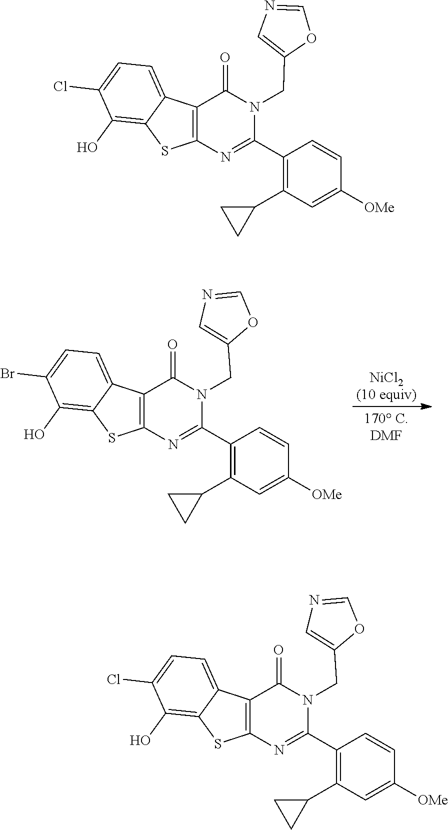 [SMILES: COc1ccc(-c2nc3sc4c(O)c(Cl)ccc4c3c(=O)n2Cc2cnco2)c(C2CC2)c1 ; COc1ccc(-c2nc3sc4c(O)c(Br)ccc4c3c(=O)n2Cc2cnco2)c(C2CC2)c1 ; COc1ccc(-c2nc3sc4c(O)c(Cl)ccc4c3c(=O)n2Cc2cnco2)c(C2CC2)c1]

To a microwave reactor was charged compound 7-bromo-2-(2-cyclopropyl-4-methoxyphenyl)-8-hydroxy-3-(oxazol-5-ylmethyl)benzo[4,5]thieno[2,3-d]pyrimidin-4(3H)-one (40 mg, 0.076 mmol), nickel dichloride (99 mg, 0.763 mmol), and DMF (2 mL). The mixture was irradiated at 170° C. under microwave for 30 mins. Upon completion, the mixture was diluted with EtOAc (5 mL) and water (5 mL). The organic layer was separated and washed with water (3×5 mL). The organic layer was then concentrated in vacuo and purified by preparative HPLC to afford compound 7-chloro-2-(2-cyclopropyl-4-methoxyphenyl)-8-hydroxy-3-(oxazol-5-ylmethyl)benzo[4,5]thieno[2,3-d]pyrimidin-4(3H)-one as a white solid (6.0 mg, 16% yield). LC-MS observed [M+H], 480.1, 482.1. ¹H NMR (400 MHz, DMSO) δ 10.82 (br, 1H), 8.21 (s, 1H), 8.00 (d, J=8.4 Hz, 1H), 7.58 (d, J=8.4 Hz, 1H), 7.36 (d, J=8.5 Hz, 1H), 6.87 (dd, J=8.5, 2.5 Hz, 1H), 6.84 (s, 1H), 6.47 (d, J=2.5 Hz, 1H), 5.39 (d, J=15.9 Hz, 1H), 5.08 (d, J=15.9, 1H), 3.18 (s, 1H), 1.58 (tt, J=8.0, 3.7 Hz, 1H), 0.85-0.74 (m, 3H), 0.68-0.56 (m, 1H).

### Synthesis of 7-chloro-2-(2-cyclopropyl-4-methoxyphenyl)-3-(oxazol-5-ylmethyl)-4-oxo-3,4-dihydrobenzo[4,5]thieno[2,3-d]pyrimidin-8-yl acetate (example 27)

### Example 27

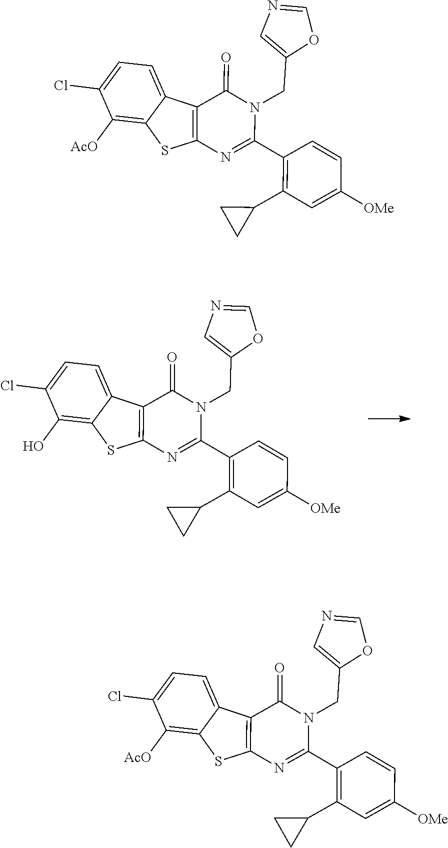 [SMILES: COc1ccc(-c2nc3sc4c(OC(C)=O)c(Cl)ccc4c3c(=O)n2Cc2cnco2)c(C2CC2)c1 ; COc1ccc(-c2nc3sc4c(O)c(Cl)ccc4c3c(=O)n2Cc2cnco2)c(C2CC2)c1 ; COc1ccc(-c2nc3sc4c(OC(C)=O)c(Cl)ccc4c3c(=O)n2Cc2cnco2)c(C2CC2)c1]

To a 4-dram vial was charged 7-chloro-2-(2-cyclopropyl-4-methoxyphenyl)-8-hydroxy-3-(oxazol-5-ylmethyl)benzo[4,5]thieno[2,3-d]pyrimidin-4(3H)-one (113 mg, 1 Eq, 235 mol) and Pyridine (3 mL) followed by the addition of acetic anhydride (120 mg, 111 μL, 5 Eq, 1.18 mmol). The mixture was allowed to stir at rt overnight. Concentrated and purified by Prep HPLC to give 7-chloro-2-(2-cyclopropyl-4-methoxyphenyl)-3-(oxazol-5-ylmethyl)-4-oxo-3,4-dihydrobenzo[4,5]thieno[2,3-d]pyrimidin-8-yl acetate (81 mg, 66% yield) as a white solid. LC-MS observed [M+H], 522.15. ¹H NMR (400 MHz, DMSO) δ 8.42 (d, J=8.5 Hz, 1H), 8.22 (s, 1H), 7.83 (d, J=8.6 Hz, 1H), 7.37 (d, J=8.5 Hz, 1H), 6.88 (dd, J=8.5, 2.5 Hz, 1H), 6.86 (s, 1H), 6.48 (d, J=2.5 Hz, 1H), 5.41 (d, J=16.0 Hz, 1H), 5.12 (d, J=15.9 Hz, 1H), 3.81 (s, 3H), 2.50 (s, 3H), 1.66-1.55 (m, 1H), 0.85-0.78 (m, 3H), 0.64 (t, J=5.0 Hz, 1H).

### Synthesis of 7-amino-2-(2-cyclopropyl-4-methoxyphenyl)-8-hydroxy-3-(oxazol-5-ylmethyl)benzo[4,5]thieno[2,3-d]pyrimidin-4(3H)-one (example 28)

### Example 28

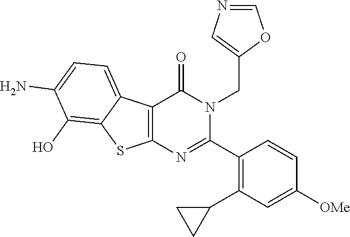 [SMILES: COc1ccc(-c2nc3sc4c(O)c(N)ccc4c3c(=O)n2Cc2cnco2)c(C2CC2)c1]

### Step 1

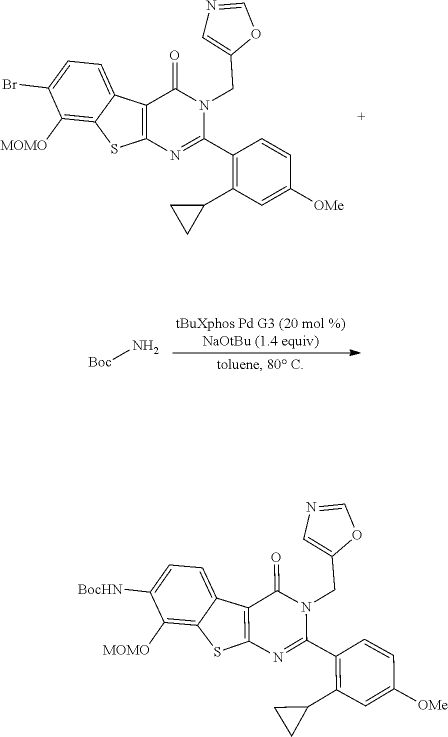

To a 2-dram vial was charged tBuXPhos Pd G3 (0.014 g, 0.018 mmol), compound 7-bromo-2-(2-cyclopropyl-4-methoxyphenyl)-8-(methoxymethoxy)-3-(oxazol-5-ylmethyl)benzo[4,5]thieno[2,3-d]pyrimidin-4(3H)-one (0.05 g, 0.088 mmol), tert-butyl carbamate (0.012 g, 0.106 mmol), and NaOtBu (0.012 g, 0.123 mmol). The vial was sealed and degassed by evacuating and filling three times with nitrogen. Next, toluene (1 mL) was added through syringe. The mixture was stirred at 80° C. for 18 hours. Upon completion, the mixture was diluted with EtOAc (5 mL) and water (5 mL). The organic layer was separated and washed with brine (5 mL). The organic layer was then concentrated in vacuo and purified by preparative HPLC to afford compound tert-butyl (2-(2-cyclopropyl-4-methoxyphenyl)-8-(methoxymethoxy)-3-(oxazol-5-ylmethyl)-4-oxo-3,4-dihydrobenzo[4,5]thieno[2,3-d]pyrimidin-7-yl)carbamate as a yellow solid (24 mg, 45% yield), which is used directly in the next step. LC-MS observed [M+H], 605.4.

### Step 2

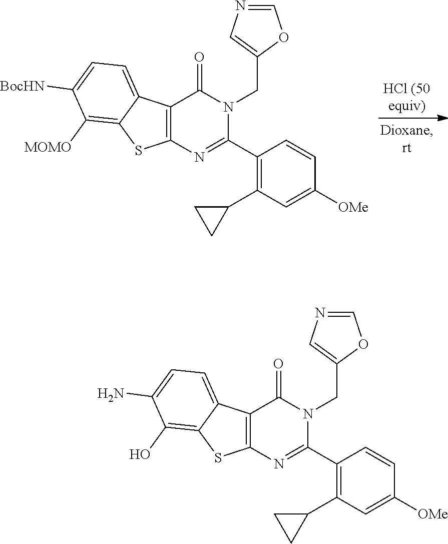

To a 2-dram vial was charged compound tert-butyl (2-(2-cyclopropyl-4-methoxyphenyl)-8-(methoxymethoxy)-3-(oxazol-5-ylmethyl)-4-oxo-3,4-dihydrobenzo[4,5]thieno[2,3-d]pyrimidin-7-yl)carbamate (0.026 g, 0.043 mmol), 4 M HCl in dioxane (0.53 mL), and dioxane (0.5 mL). The mixture was stirred at room temperature for 18 hours. Upon completion, the mixture was evaporated to dryness under vacuum. The residue was purified by preparative HPLC to afford compound 7-amino-2-(2-cyclopropyl-4-methoxyphenyl)-8-hydroxy-3-(oxazol-5-ylmethyl)benzo[4,5]thieno[2,3-d]pyrimidin-4(3H)-one as a green solid (12 mg, 61% yield). LC-MS observed [M+H], 461.3. ¹H NMR (400 MHz, DMSO) δ 8.20 (s, 1H), 7.83 (d, J=8.3 Hz, 1H), 7.33 (d, J=8.5 Hz, 1H), 6.93 (d, J=8.3 Hz, 1H), 6.85 (dd, J=8.5, 2.5 Hz, 1H), 6.81 (s, 1H), 6.46 (d, J=2.5 Hz, 1H), 5.38 (d, J=15.8 Hz, 1H), 5.04 (d, J=15.9 Hz, 1H), 3.80 (s, 3H), 2.54 (br, 3H), 1.59-1.47 (m, 1H), 0.88-0.75 (m, 3H), 0.65-0.56 (m, 1H).

### Synthesis of 8-(2-cyclopropyl-4-methoxyphenyl)-7-(oxazol-5-ylmethyl)-3,7-dihydrooxazolo[5″,4″:3′,4′]benzo[1′,2′:4,5]thieno[2,3-d]pyrimidine-2,6-dione (example 29)

### Example 29

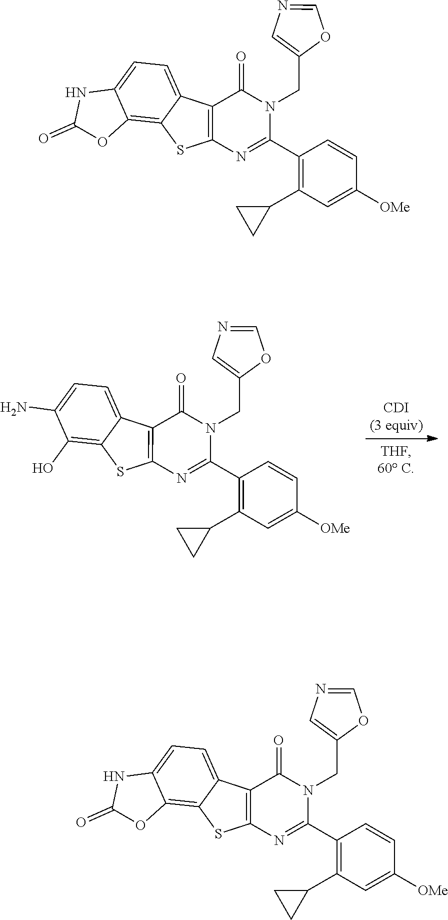 [SMILES: COc1ccc(-c2nc3sc4c(ccc5[nH]c(=O)oc54)c3c(=O)n2Cc2cnco2)c(C2CC2)c1 ; COc1ccc(-c2nc3sc4c(O)c(N)ccc4c3c(=O)n2Cc2cnco2)c(C2CC2)c1 ; COc1ccc(-c2nc3sc4c(ccc5[nH]c(=O)oc54)c3c(=O)n2Cc2cnco2)c(C2CC2)c1]

In a 2-dram vial containing compound 7-amino-2-(2-cyclopropyl-4-methoxyphenyl)-8-hydroxy-3-(oxazol-5-ylmethyl)benzo[4,5]thieno[2,3-d]pyrimidin-4(3H)-one (8 mg, 0.017 mmol) was added CDI (8.5 mg, 0.052 mmol), and THF (1 mL). The resulting solution was stirred at 60° C. for 1 hour. Then, the mixture was evaporated to dryness under vacuum. The residue was purified by preparative HPLC to afford compound 8-(2-cyclopropyl-4-methoxyphenyl)-7-(oxazol-5-ylmethyl)-3,7-dihydrooxazolo[5″,4″:3′,4′]benzo[1′,2′:4,5]thieno[2,3-d]pyrimidine-2,6-dione as a white solid (6.7 mg, 79% yield). LC-MS observed [M+H], 487.2. ¹H NMR (400 MHz, DMSO) δ 12.10 (br, 1H), 8.37 (d, J=8.4, 1H), 8.22 (s, 1H), 7.42-7.35 (m, 2H), 6.88 (dd, J=8.6, 2.5 Hz, 1H), 6.85 (s, 1H), 6.48 (d, J=2.5 Hz, 1H), 5.41 (d, J=15.9 Hz, 1H), 5.11 (d, J=15.9 Hz, 1H), 3.81 (s, 3H), 1.64-1.53 (m, 1H), 0.87-0.75 (m, 3H), 0.67-0.58 (m, 1H).

### Synthesis of 8-(2-cyclopropyl-4-methoxyphenyl)-7-(oxazol-5-ylmethyl)-3,7-dihydrooxazolo[5″,4″:3′,4′]benzo[1′,2′:4,5]thieno[2,3-d]pyrimidine-2,6-dione (example 30)

### Example 30

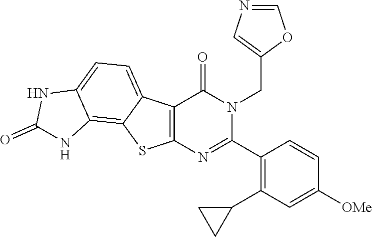 [SMILES: COc1ccc(-c2nc3sc4c(ccc5[nH]c(=O)[nH]c54)c3c(=O)n2Cc2cnco2)c(C2CC2)c1]

### Step 1

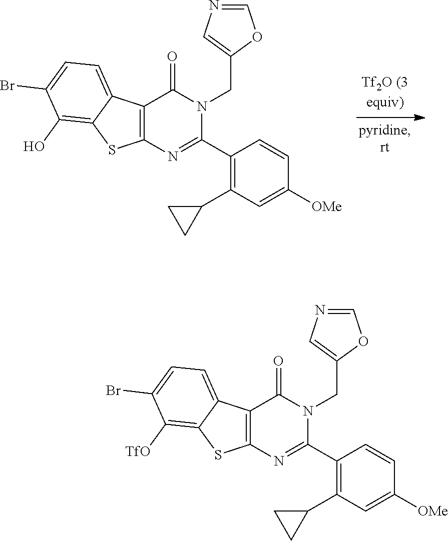 [SMILES: COc1ccc(-c2nc3sc4c(O)c(Br)ccc4c3c(=O)n2Cc2cnco2)c(C2CC2)c1 ; COc1ccc(-c2nc3sc4c(OS(=O)(=O)C(F)(F)F)c(Br)ccc4c3c(=O)n2Cc2cnco2)c(C2CC2)c1]

To a stirred solution of compound 7-bromo-2-(2-cyclopropyl-4-methoxyphenyl)-8-hydroxy-3-(oxazol-5-ylmethyl)benzo[4,5]thieno[2,3-d]pyrimidin-4(3H)-one (0.10 g, 0.19 mmol) in pyridine (2 mL) was slowly charged Tf₂O (0.097 mL, 0.57 mmol) at 0° C. The reaction was stirred at rt for 18 hours. Upon completion, the reaction was diluted with ethyl acetate (10 mL), washed with Sat. NaHCO₃ (10 mL), water (10 mL), and brine (10 mL). The organic layer was dried, filtered, concentrated, and purified by CombiFlash (4 g SiO₂, EtOAc/Cyclohexane: 0˜30%) to afford compound 7-bromo-2-(2-cyclopropyl-4-methoxyphenyl)-3-(oxazol-5-ylmethyl)-4-oxo-3,4-dihydrobenzo[4,5]thieno[2,3-d]pyrimidin-8-yl trifluoromethanesulfonate as a white solid (0.11 g, 89% yield), which is used directly in the next step. LC-MS observed [M+H], 656.0, 658.0.

### Step 2

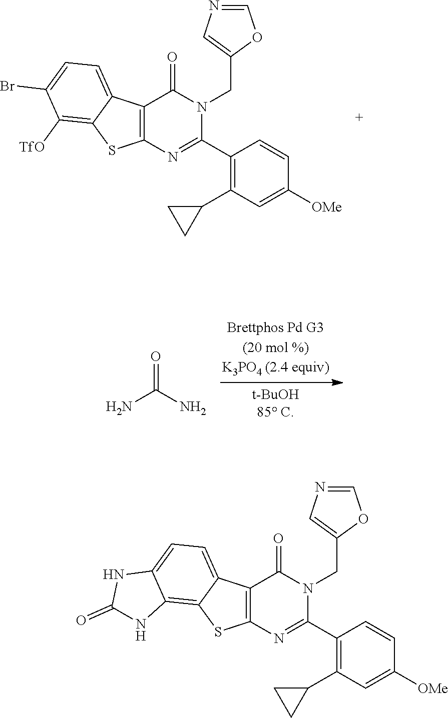

A 4-dram vial with a magnetic stir bar was charged with Brettphos Pd G3 (14 mg, 0.015 mmol), 7-bromo-2-(2-cyclopropyl-4-methoxyphenyl)-3-(oxazol-5-ylmethyl)-4-oxo-3,4-dihydrobenzo[4,5]thieno[2,3-d]pyrimidin-8-yl trifluoromethanesulfonate (50 mg, 0.076 mmol), urea (23 mg, 0.38 mmol) and potassium phosphate (39 mg, 0.18 mmol). The tube was evacuated and backfilled with nitrogen and this process was repeated twice. t-BuOH (0.5 ml) was added via syringe and the punctured cap was replaced with a new one under a stream of nitrogen. The vial was heated at 85° C. and stirred for 18 h. Upon completion, the reaction was diluted with ethyl acetate (5 mL), washed with Sat. NH₄Cl (5 mL), water (5 mL), and brine (5 mL). The organic layer was dried, filtered, concentrated, and purified by preparative HPLC to afford compound 8-(2-cyclopropyl-4-methoxyphenyl)-7-(oxazol-5-ylmethyl)-3,7-dihydro-1H-imidazo[4″,5″:3′,4′]benzo[1′,2′:4,5]thieno[2,3-d]pyrimidine-2,6-dione as a white solid (4.3 mg, 12% yield). LC-MS observed [M+H], 486.3. ¹H NMR (400 MHz, MeOD) δ 8.38 (d, J=8.4, 1H), 8.07 (s, 1H), 7.33 (d, J=1.6 Hz, 1H), 7.31 (d, J=1.5 Hz, 1H), 6.91 (dd, J=8.5, 2.5 Hz, 1H), 6.85 (s, 1H), 6.56 (d, J=2.5 Hz, 1H), 5.55 (d, J=15.8 Hz, 1H), 5.23 (d, J=15.7 Hz, 1H), 3.86 (s, 3H), 1.63-1.53 (m, 1H), 0.95-0.75 (m, 3H), 0.72-0.62 (m, 1H).

### Synthesis of 2-(2-cyclopropyl-4-methoxyphenyl)-8-hydroxy-3-(oxazol-5-ylmethyl)-4-oxo-3,4-dihydrobenzo[4,5]thieno[2,3-d]pyrimidine-7-carbaldehyde (example 31)

### Example 31

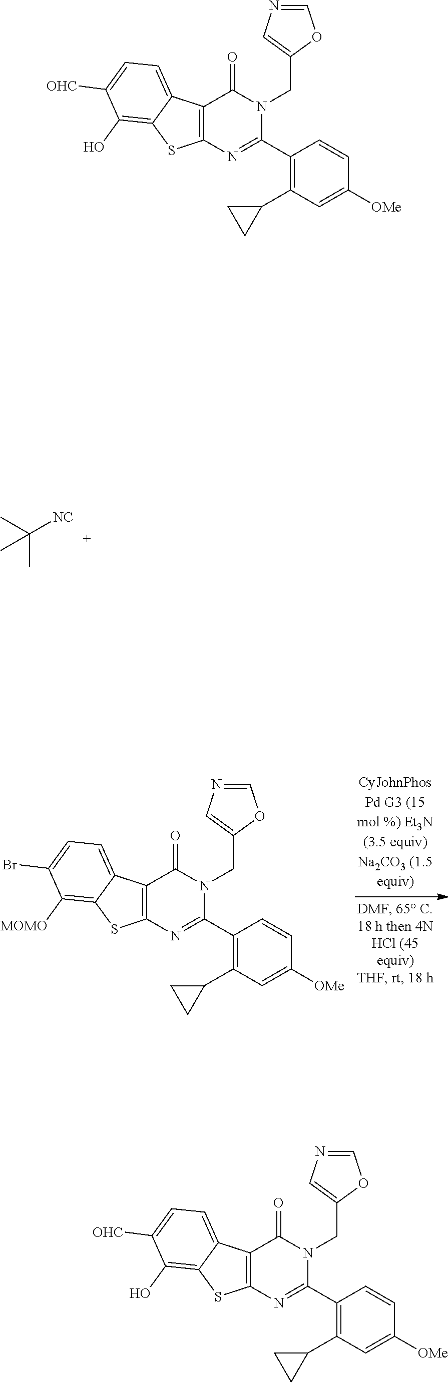

Compound 7-bromo-2-(2-cyclopropyl-4-methoxyphenyl)-8-(methoxymethoxy)-3-(oxazol-5-ylmethyl)benzo[4,5]thieno[2,3-d]pyrimidin-4(3H)-one (0.050 g, 88 μmol), tert-butyl isocyanide (11 mg, 1.5 Eq, 0.13 mmol), CyJohnPhos Pd G3 (9.5 mg, 0.15 Eq, 13 μmol), triethylsilane (36 mg, 3.5 Eq, 0.31 mmol), sodium carbonate (14 mg, 1.5 Eq, 0.13 mmol) and DMF (1 mL) were added to a 2-dram vial, and stirred at 65° C. for 8 h under nitrogen. After the reaction was complete, the crude mixture was quenched with water and diluted with ethyl acetate (3 mL). The organic layer was separated and filtrated through a thin pad of silica gel, washed with ethyl acetate (2 mL). The filtrate was concentrated and dissolved in 1 mL THF followed by the addition of hydrogen chloride (0.14 g, 0.99 mL, 4 molar, 45 eq, 4.0 mmol). The mixture was stirred at RT for O/N. After the deprotection was complete, the mixture was evaporated under vacuum to dryness, the residue was purified by column to give the crude product. The crude product was then purified by preparative HPLC to afford compound 2-(2-cyclopropyl-4-methoxyphenyl)-8-hydroxy-3-(oxazol-5-ylmethyl)-4-oxo-3,4-dihydrobenzo[4,5]thieno[2,3-d]pyrimidine-7-carbaldehyde as a yellow solid (9.8 mg, 12% yield). LC-MS observed [M+H], 474.2. ¹H NMR (400 MHz, DMSO) δ 11.72 (s, 1H), 10.27 (s, 1H), 8.23 (s, 1H), 8.18 (d, J=8.3 Hz, 1H), 7.93 (d, J=8.3 Hz, 1H), 7.39 (d, J=8.5 Hz, 1H), 6.89 (dd, J=8.6, 2.5 Hz, 1H), 6.87 (s, 1H), 6.49 (d, J=2.5 Hz, 1H), 5.40 (d, J=15.9 Hz, 1H), 5.11 (d, J=15.9 Hz, 1H), 3.82 (s, 3H), 1.67-1.57 (m, 1H), 0.83 (m, 3H), 0.64 (d, J=4.7 Hz, 1H).

### Synthesis of 2-(2-cyclopropyl-4-methoxyphenyl)-8-hydroxy-7-methyl-3-(oxazol-5-ylmethyl)benzo[4,5]thieno[2,3-d]pyrimidin-4(3H)-one (example 32)

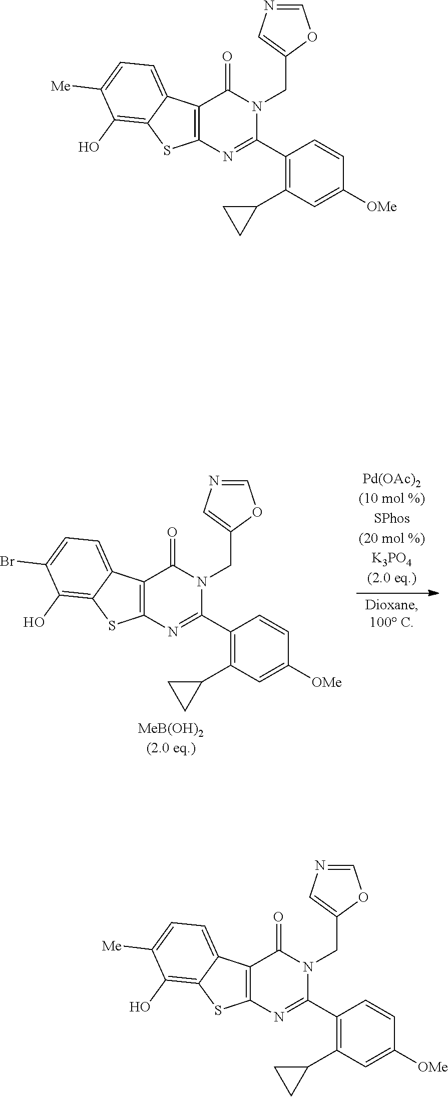 [SMILES: COc1ccc(-c2nc3sc4c(O)c(C)ccc4c3c(=O)n2Cc2cnco2)c(C2CC2)c1 ; COc1ccc(-c2nc3sc4c(O)c(Br)ccc4c3c(=O)n2Cc2cnco2)c(C2CC2)c1 ; COc1ccc(-c2nc3sc4c(O)c(C)ccc4c3c(=O)n2Cc2cnco2)c(C2CC2)c1]

To a vial, potassium phosphate (0.042 g, 0.200 mmol, 2.0 eq.), palladium(II) acetate (2.245 mg, 10.00 μmol, 0.1 eq.), 7-bromo-2-(2-cyclopropyl-4-methoxyphenyl)-8-hydroxy-3-(oxazol-5-ylmethyl)benzo[4,5]thieno[2,3-d]pyrimidin-4(3H)-one (0.052 g, 0.1 mmol, 1 eq.), methylboronic acid (0.012 g, 0.200 mmol, 2.0 eq.) and SPhos (8.21 mg, 0.020 mmol, 0.2 eq.) were added respectively and the mixture was evacuated and backfilled with nitrogen three times. Dioxane (1 mL) was added and the mixture was heated at 100° C. The reaction progress was monitored with LCMS. Upon completion, the reaction mixture was diluted with EtOAc. The organic layer was washed with water and brine respectively. Dried (Na₂SO₄), filtered, concentrated, and purified with prep-HPLC to afford 2-(2-cyclopropyl-4-methoxyphenyl)-8-hydroxy-7-methyl-3-(oxazol-5-ylmethyl)benzo[4,5]thieno[2,3-d]pyrimidin-4(3H)-one (10 mg, 21.76% yield). LC-MS observed [M+H], 460.3. ¹H NMR (400 MHz, DMSO) δ 8.20 (s, 1H), 7.94 (d, J=7.9 Hz, 1H), 7.34 (dd, J=10.3, 8.2 Hz, 2H), 6.86 (dd, J=8.5, 2.5 Hz, 1H), 6.83 (s, 1H), 6.46 (d, J=2.5 Hz, 1H), 5.39 (d, J=15.9 Hz, 1H), 5.07 (d, J=15.9 Hz, 1H), 3.80 (s, 3H), 2.36 (s, 3H), 1.61-1.49 (m, 1H), 0.82 (dq, J=6.8, 3.0 Hz, 3H), 0.67-0.54 (m, 1H).

### Synthesis of 2-(2-cyclopropyl-4-methoxyphenyl)-8-hydroxy-3-(oxazol-5-ylmethyl)-4-oxo-3,4-dihydrobenzo[4,5]thieno[2,3-d]pyrimidine-7-carbonitrile (example 33)

### Example 33

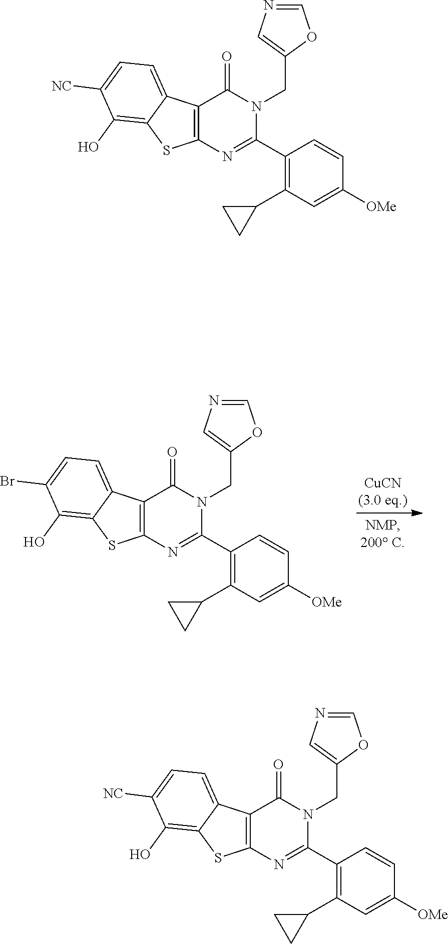 [SMILES: COc1ccc(-c2nc3sc4c(O)c(C#N)ccc4c3c(=O)n2Cc2cnco2)c(C2CC2)c1 ; COc1ccc(-c2nc3sc4c(O)c(Br)ccc4c3c(=O)n2Cc2cnco2)c(C2CC2)c1 ; COc1ccc(-c2nc3sc4c(O)c(C#N)ccc4c3c(=O)n2Cc2cnco2)c(C2CC2)c1]

To a MW vial of were added copper(I) cyanide (36.9 mg, 0.412 mmol, 3.0 eq.), 7-bromo-2-(2-cyclopropyl-4-methoxyphenyl)-8-hydroxy-3-(oxazol-5-ylmethyl)benzo[4,5]thieno[2,3-d]pyrimidin-4(3H)-one (72 mg, 0.137 mmol, 1.0 eq.) and N-Methyl-2-pyrrolidinone (0.5 mL) respectively and the mixture was irradiated at 200° C. under microwave for 1 h. The reaction was diluted with EtOAc and the organic layer was washed with sat. NaHCO₃(aq.) and then brine. The organic layer was dried over Na₂SO₄, filtered, concentrated, and purified with prep-HPLC to afford 2-(2-cyclopropyl-4-methoxyphenyl)-8-hydroxy-3-(oxazol-5-ylmethyl)-4-oxo-3,4-dihydrobenzo[4,5]thieno[2,3-d]pyrimidine-7-carbonitrile (1.3 mg, 2.0% yield). LC-MS observed [M+H], 471.2. ¹H NMR (400 MHz, MeOD) δ 8.40 (s, 0.5H, —OH), 8.07 (s, 1H), 7.96 (d, J=8.3 Hz, 1H), 7.55 (d, J=8.3 Hz, 1H), 7.31 (d, J=8.5 Hz, 1H), 6.90 (dd, J=8.5, 2.5 Hz, 1H), 6.84 (s, 1H), 6.55 (d, J=2.5 Hz, 1H), 5.52 (d, J=15.8 Hz, 1H), 5.21 (d, J=15.7 Hz, 1H), 3.86 (s, 3H), 1.58 (ddd, J=13.4, 8.4, 5.1 Hz, 1H), 0.93-0.74 (m, 3H), 0.67 (dt, J=9.6, 5.0 Hz, 1H).

### Synthesis of 2-(2-cyclopropyl-4-methoxyphenyl)-8-hydroxy-3-(oxazol-5-ylmethyl)-4-oxo-3,4-dihydrobenzo[4,5]thieno[2,3-d]pyrimidine-7-carboxylic acid (example 34)

### Example 34

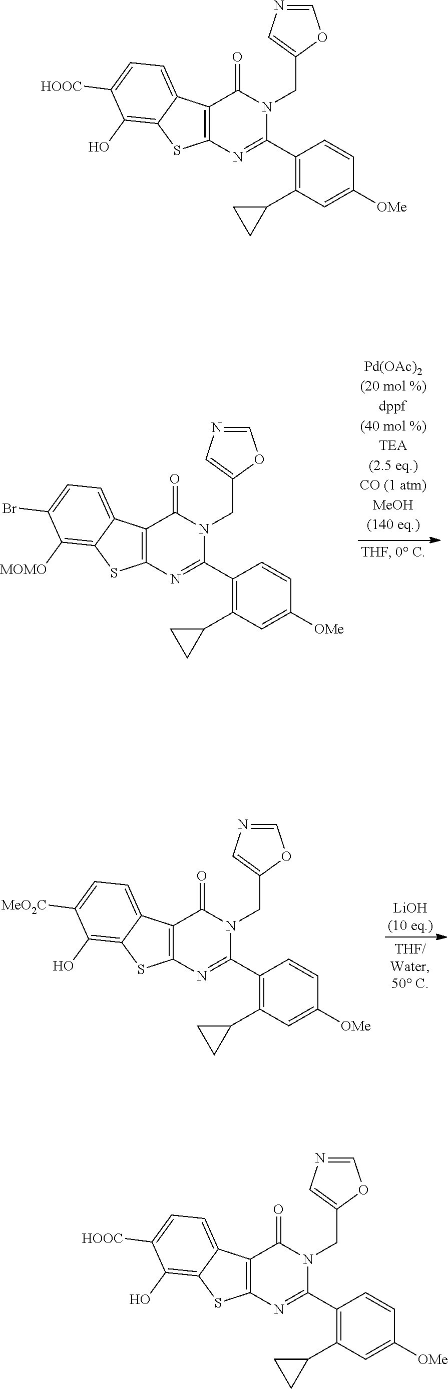

### Step 1

To a sealed vial, was charged with 7-bromo-2-(2-cyclopropyl-4-methoxyphenyl)-8-(methoxymethoxy)-3-(oxazol-5-ylmethyl)benzo[4,5]thieno[2,3-d]pyrimidin-4(3H)-one (50 mg, 0.088 mmol, 1.0 eq.), DMF (1 mL) and methanol (498 μL, 12.31 mmol, 140 eq.). The sealed vial was evacuated and backfilled with nitrogen followed by the addition of dppf (19.51 mg, 0.035 mmol, 0.4 eq.) and diacetoxypalladium (3.95 mg, 0.018 mmol, 0.2 eq.). The nitrogen atmosphere was exchanged with carbon monoxide (1 atm, balloon) 3 minutes was then added triethylamine (30.7 μL, 0.220 mmol). The mixture was stirred at 80° C. overnight. The mixture was diluted with EtOAc then the organic layer was washed with water and brine. The residue was purified through column chromatography to afford methyl 2-(2-cyclopropyl-4-methoxyphenyl)-8-hydroxy-3-(oxazol-5-ylmethyl)-4-oxo-3,4-dihydrobenzo[4,5]thieno[2,3-d]pyrimidine-7-carboxylate (10 mg, 23% yield). LC-MS observed [M+H], 504.1.

### Step 2

To the flask with methyl 2-(2-cyclopropyl-4-methoxyphenyl)-8-hydroxy-3-(oxazol-5-ylmethyl)-4-oxo-3,4-dihydrobenzo[4,5]thieno[2,3-d]pyrimidine-7-carboxylate (10 mg, 1 Eq, 20 μmol) in THF (4 mL) were added water (2 mL) and lithium hydroxide monohydrate (8.2 mg, 5.4 μL, 10 Eq, 0.20 mmol). The mixture was heated to 50° C. After overnight, the reaction was quenched by the addition of ethyl acetate. 4M HCl was added dropwise until pH of aq. layer reaches ˜2. The aq. layer was extracted one more time with ethyl acetate. The combined organic layer was washed with water and brine. Dried over Na₂SO₄, filtered, concentrated, and purified through prep-HPLC to afford 2-(2-cyclopropyl-4-methoxyphenyl)-8-hydroxy-3-(oxazol-5-ylmethyl)-4-oxo-3,4-dihydrobenzo[4,5]thieno[2,3-d]pyrimidine-7-carboxylic acid (3.5 mg, 36% yield). LC-MS observed [M+H], 490.1. ¹H NMR (400 MHz, MeOD) δ 8.12-8.00 (m, 3H), 7.34 (d, J=8.5 Hz, 1H), 6.92 (dd, J=8.5, 2.5 Hz, 1H), 6.86 (s, 1H), 6.57 (d, J=2.5 Hz, 1H), 5.55 (d, J=15.8 Hz, 1H), 5.24 (d, J=15.8 Hz, 1H), 3.88 (s, 3H), 1.60 (td, J=8.4, 4.2 Hz, 1H), 1.01-0.74 (m, 3H), 0.69 (dt, J=10.0, 5.1 Hz, 1H).

### Synthesis of 2-(2-cyclopropyl-4-methoxyphenyl)-8-hydroxy-3-(oxazol-5-ylmethyl)-7-phenylbenzo[4,5]thieno[2,3-d]pyrimidin-4(3H)-one (example 35)

### Example 35

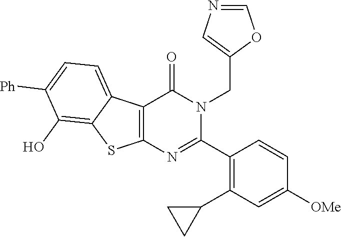 [SMILES: COc1ccc(-c2nc3sc4c(O)c(-c5ccccc5)ccc4c3c(=O)n2Cc2cnco2)c(C2CC2)c1]

### Step 1

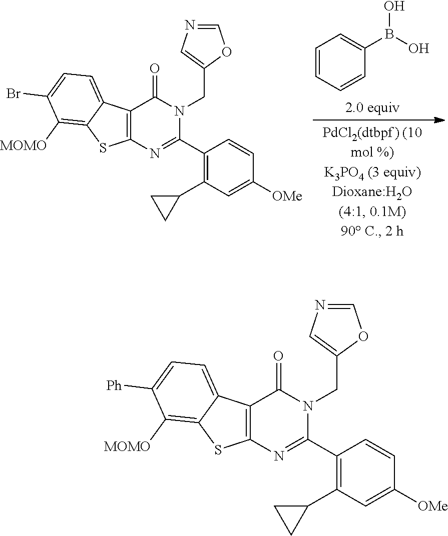

In a 2 mL vial equipped with stir bar, 7-bromo-2-(2-cyclopropyl-4-methoxyphenyl)-8-(methoxymethoxy)-3-(oxazol-5-ylmethyl)benzo[4,5]thieno[2,3-d]pyrimidin-4(3H)-one (25 mg, 1.0 equiv.), phenylboronic acid (11 mg, 2.0 equiv.), potassium phosphate tribasic (28 mg, 3 equiv.) and [1,1′-bis(di-tert-butylphosphino) ferrocene]dichloropalladium(II) (2.9 mg, 0.1 equiv.) were combined neat under nitrogen atmosphere, followed by addition of 1,4-Dioxane (0.35 mL) and water (88 μL). The reaction mixture was heated at 90° C. for 2 h in a heating block to afford 2-(2-cyclopropyl-4-methoxyphenyl)-8-(methoxymethoxy)-3-(oxazol-5-ylmethyl)-7-phenylbenzo[4,5]thieno[2,3-d]pyrimidin-4(3H)-one. ESI MS m/z=566.4 [M+H]⁺.

### Step 2

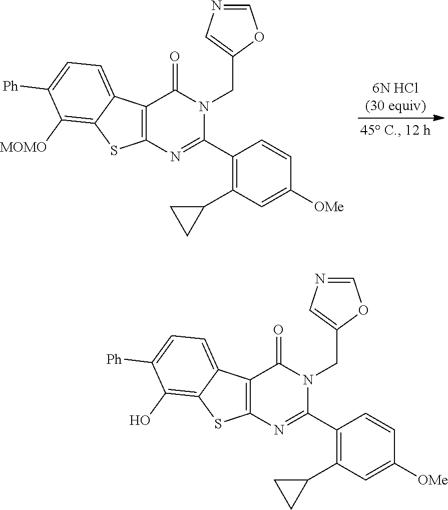

In the reaction mixture from step 1 was added 6N HCl (0.22 mL, 30 equiv.) and heated at 45° C. for 12 h in a heating block. Then, the crude mixture was concentrated under vacuum and the residue was re-dissolved in 2.0 mL of dimethyl sulfoxide, passed through a 0.45 μm syringe filter, and purified by RPHPLC to afford 2-(2-cyclopropyl-4-methoxyphenyl)-8-hydroxy-3-(oxazol-5-ylmethyl)-7-phenylbenzo[4,5]thieno[2,3-d]pyrimidin-4(3H)-one (6 mg, 30% yield). ESI MS m/z=522.2 [M+H]⁺. ¹H NMR (500 MHz, DMSO-d₆) δ 8.22 (s, 1H), 8.10 (d, J=8.1 Hz, 1H), 7.66-7.61 (m, 2H), 7.52-7.44 (m, 3H), 7.40-7.33 (m, 2H), 6.88 (dd, J=8.5, 2.5 Hz, 1H), 6.84 (s, 1H), 6.48 (d, J=2.5 Hz, 1H), 5.41 (d, J=15.9 Hz, 1H), 5.10 (d, J=15.9 Hz, 1H), 3.81 (s, 3H), 1.58 (tt, J=7.8, 5.6 Hz, 1H), 0.89-0.77 (m, 3H), 0.69-0.58 (m, 1H).

### Synthesis of 2-(2-cyclopropyl-4-methoxyphenyl)-7-hydroxy-3-(oxazol-5-ylmethyl)benzo[4,5]thieno[2,3-d]pyrimidin-4(3H)-one (example 36) and 8-bromo-2-(2-cyclopropyl-4-methoxyphenyl)-7-hydroxy-3-(oxazol-5-ylmethyl)benzo[4,5]thieno[2,3-d]pyrimidin-4(3H)-one

### Example 36

### Example 37

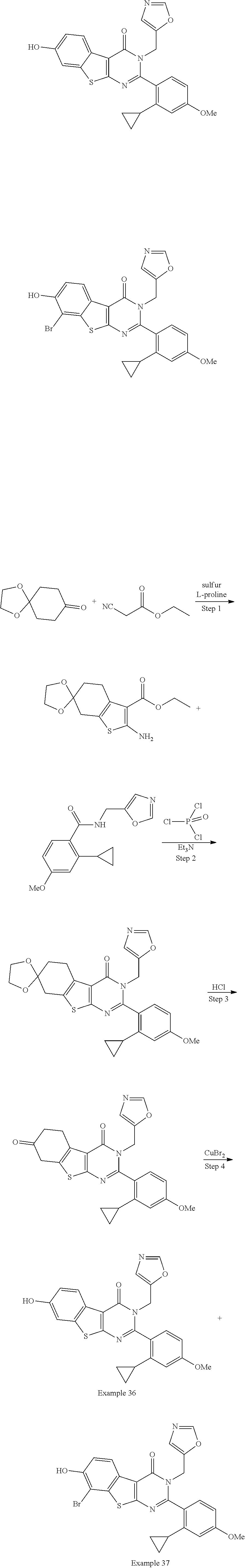

### Step 1

To a 25 mL round bottom flask was added 1,4-dioxaspiro[4.5]decan-8-one (1.4 g, 8.8 mmol), ethyl 2-cyanoacetate (0.95 mL, 8.8 mmol), sulfur powder (0.34 g, 10.6 mmol), L-proline (0.10 g, 0.88 mmol), and DMF (8 mL) respectively and the solution was stirred at 60° C. for 20 h. Cooled to rt, the solution was slowly added to 50 mL of water with vigorous stirring. The mixture was extracted with 50% EtOAc in heptane (30 mL×3), the combined organic phase was dried. Filtered, concentrated, the residue was subjected to silica gel column, eluted with 20˜30% EtOAc in cyclohexane to give ethyl 2-amino-4,7-dihydro-5H-spiro[benzo[b]thiophene-6,2′-[1,3]dioxolane]-3-carboxylate (2.26 g, 90% yield) as a yellow solid. LC-MS observed [M+H], 284.155. ¹H NMR (400 MHz, CDCl₃) δ 5.49 (s, 2H), 4.27 (q, J=7.1 Hz, 2H), 4.03 (s, 4H), 2.94 (tt, J=6.6, 1.8 Hz, 2H), 2.75 (d, J=1.8 Hz, 2H), 1.91 (t, J=6.5 Hz, 2H), 1.34 (t, J=7.1 Hz, 3H).

### Step 2

To a 25 mL round-bottomed flask were added 2-cyclopropyl-4-methoxy-N-(oxazol-5-ylmethyl)benzamide (0.40 g, 1.47 mmol), ethyl 2-amino-4,7-dihydro-5H-spiro[benzo[b]thiophene-6,2′-[1,3]dioxolane]-3-carboxylate (0.42 g, 1.47 mmol), DCE (10 mL), triethylamine (1.0 mL, 7.35 mmol) and phosphoryl trichloride (0.35 mL, 3.68 mmol) respectively and the suspension was stirred at 80° C. for 18 h. The mixture was diluted with DCM and the insoluable brown solid was collected off by a Buchi funnel. The filtrate was washed thoroughly with Sat. NaHCO₃ and then washed with brine. The organic layer was dried (Na₂SO₄), filtered, concentrated, and used in next step without further purification.

### Step 3

To the above-mentioned residue was added acetone (8 mL), 2M HCl (0.8 mL), and the solution was stirred at 70° C. for 2 h. Cooled to rt, concentrated, and extracted with DCM 3 times. The combined DCM phase was washed with sat. NaHCO₃ solution and then brine. The organic layer was dried (Na₂SO₄), filtered, concentrated, and purified by CombiFlash (12 g SiO₂, EtOAc/Cyclohexane: 30-55%) to give 2-(2-cyclopropyl-4-methoxyphenyl)-3-(oxazol-5-ylmethyl)-3,5,6,8-tetrahydrobenzo[4,5]thieno[2,3-d]pyrimidine-4,7-dione as a yellow solid (0.12 g, 19% yield). LC-MS observed [M+H], 448.266. ¹H NMR (400 MHz, CDCl₃) δ 7.82 (s, 1H), 7.31-7.21 (m, 1H), 6.87-6.83 (m, 2H), 6.50 (d, J=2.5 Hz, 1H), 5.53 (d, J=15.4 Hz, 1H), 5.02 (d, J=15.3 Hz, 1H), 3.87 (d, J=0.9 Hz, 3H), 3.68 (s, 2H), 3.63-3.44 (m, 2H), 2.77 (t, J=6.8 Hz, 2H), 1.56 (ddd, J=13.1, 8.3, 5.1 Hz, 1H), 0.90 (ddt, J=13.1, 10.4, 7.5 Hz, 3H), 0.67 (dt, J=6.5, 4.2 Hz, 1H).

### Step 4

To a 5 mL vial were added 2-(2-cyclopropyl-4-methoxyphenyl)-3-(oxazol-5-ylmethyl)-3,5,6,8-tetrahydrobenzo[4,5]thieno[2,3-d]pyrimidine-4,7-dione (0.050 g, 0.11 mmol), acetonitrile (0.75 mL) and copper (II) bromide (0.050 g, 0.22 mmol). The suspension was stirred at 60° C. for 2 h. Then allowed to cool to rt and concentrated. The residue was diluted by DCM, washed with 1M HCl and brine. The solution was concentrated and purified by CombiFlash (4 g SiO₂, EtOAc/Cyclohexane: 50-70%) to give a yellow solid which was not pure. The solid was subjected to prep-HPLC purification to give 2-(2-cyclopropyl-4-methoxyphenyl)-7-hydroxy-3-(oxazol-5-ylmethyl)benzo[4,5]thieno[2,3-d]pyrimidin-4(3H)-one (8.0 mg, 16% yield). LC-MS observed [M+H], 446.298. ¹H NMR (400 MHz, CDCl₃) δ 8.41-8.22 (m, 1H), 7.75 (s, 1H), 7.37 (dd, J=20.9, 5.3 Hz, 2H), 6.86 (d, J=14.2 Hz, 2H), 6.81-6.70 (m, 1H), 6.43 (dd, J=14.3, 2.4 Hz, 1H), 5.54 (dd, J=15.3, 12.2 Hz, 1H), 5.15-4.99 (m, 1H), 3.82-3.74 (s, 3H), 1.50 (tq, J=8.3, 4.1 Hz, 1H), 0.82 (qt, J=12.2, 5.7 Hz, 3H), 0.59 (dt, J=6.8, 4.1 Hz, 1H).

8-bromo-2-(2-cyclopropyl-4-methoxyphenyl)-7-hydroxy-3-(oxazol-5-ylmethyl)benzo[4,5]thieno[2,3-d]pyrimidin-4(3H)-one was obtained as well (4.0 mg, 8% yield). LC-MS observed [M+H], 526.027. ¹H NMR (400 MHz, d6-Acetone) δ 9.51 (s, 1H), 8.45 (d, J=8.6 Hz, 1H), 7.40 (d, J=8.5 Hz, 1H), 7.31 (d, J=8.5 Hz, 1H), 6.92 (dd, J=8.5, 2.8 Hz, 2H), 6.57 (t, J=2.6 Hz, 1H), 5.58 (d, J=15.7 Hz, 1H), 5.22 (d, J=16.3 Hz, 1H), 3.89 (s, 3H), 1.75 (h, J=6.0 Hz, 1H), 0.90 (m, 3H), 0.72 (d, J=6.8 Hz, 1H).

### Synthesis of 8-(2-cyclopropyl-4-methoxyphenyl)-7-(oxazol-5-ylmethyl)oxazolo[4″,5″:3′,4′]benzo[1′,2′:4,5]thieno[2,3-d]pyrimidine-2,6(1H,7H)-dione (example 38)

### Example 38

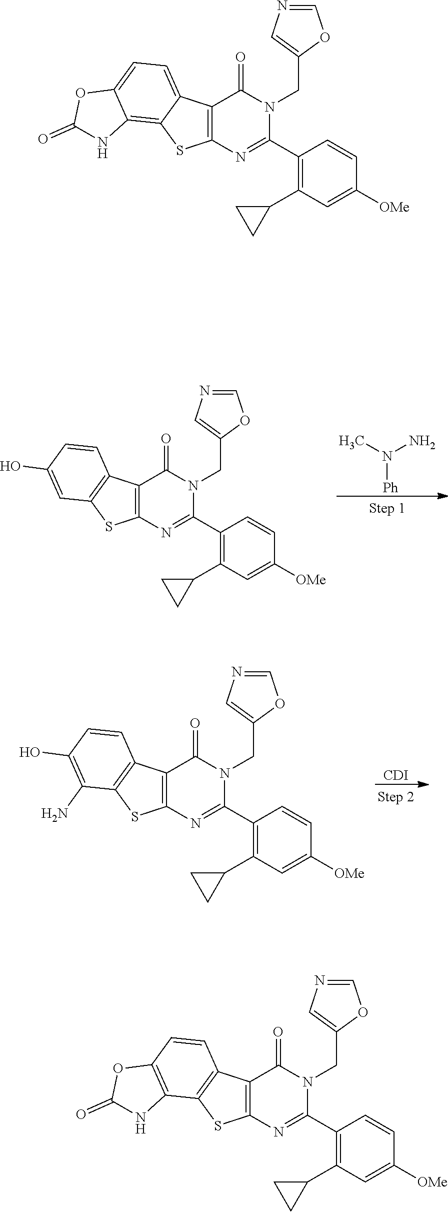

### Step 1

To a 5 mL vial were added 2-(2-cyclopropyl-4-methoxyphenyl)-7-hydroxy-3-(oxazol-5-ylmethyl)benzo[4,5]thieno[2,3-d]pyrimidin-4(3H)-one (0.023 g, 0.052 mmol), ethylene glycol (0.15 mL) and N,N-methylphenyl hydrazine (0.030 mL, 0.26 mmol) and the solution was stirred at 120° C. for 6 h. Then it was allowed to cool to rt, diluted with DMSO, and directly purified with prep-HPLC to give 8-amino-2-(2-cyclopropyl-4-methoxyphenyl)-7-hydroxy-3-(oxazol-5-ylmethyl)benzo[4,5]thieno[2,3-d]pyrimidin-4(3H)-one (7.0 mg, 0.015 mmol), which was not pure and used in next step without further purification.

### Step 2

To a 5 mL vial were added 8-amino-2-(2-cyclopropyl-4-methoxyphenyl)-7-hydroxy-3-(oxazol-5-ylmethyl)benzo[4,5]thieno[2,3-d]pyrimidin-4(3H)-one (6.0 mg, 0.013 mmol, not 100% pure), DCM (1 mL) and carbonyldiimidazole (8.4 mg, 0.052 mmol) and the solution was stirred at 60° C. for 4 h. Then allowed to cool to rt, concentrated, the residue was purified by a short plug of silica gel, eluted with 50% EtOAc in cyclohexane to give 8-(2-cyclopropyl-4-methoxyphenyl)-7-(oxazol-5-ylmethyl)oxazolo[4″,5″:3′,4′]benzo[1′,2′:4,5]thieno[2,3-d]pyrimidine-2,6(1H,7H)-dione as a white solid (1.4 mg, 22% yield). LC-MS observed [M+H], 487.098. ¹H NMR (400 MHz, d6-DMSO) δ 12.37 (s, 1H), 8.33 (d, J=8.6 Hz, 1H), 8.22 (s, 1H), 7.60 (d, J=8.6 Hz, 1H), 7.38 (d, J=8.5 Hz, 1H), 6.89 (dd, J=8.6, 2.4 Hz, 1H), 6.85 (s, 1H), 6.48 (d, J=2.5 Hz, 1H), 5.42 (d, J=15.9 Hz, 1H), 5.12 (d, J=15.9 Hz, 1H), 3.82 (s, 3H), 1.64-1.49 (m, 1H), 0.83 (dd, J=7.8, 4.7 Hz, 3H), 0.67-0.58 (m, 1H).

### Synthesis of 7-chloro-2-(2-cyclopropyl-4-methoxyphenyl)-8-hydroxy-3-((3-methylisoxazol-5-yl)methyl)benzo[4,5]thieno[2,3-d]pyrimidin-4(3H)-one (example 39)

### Example 39

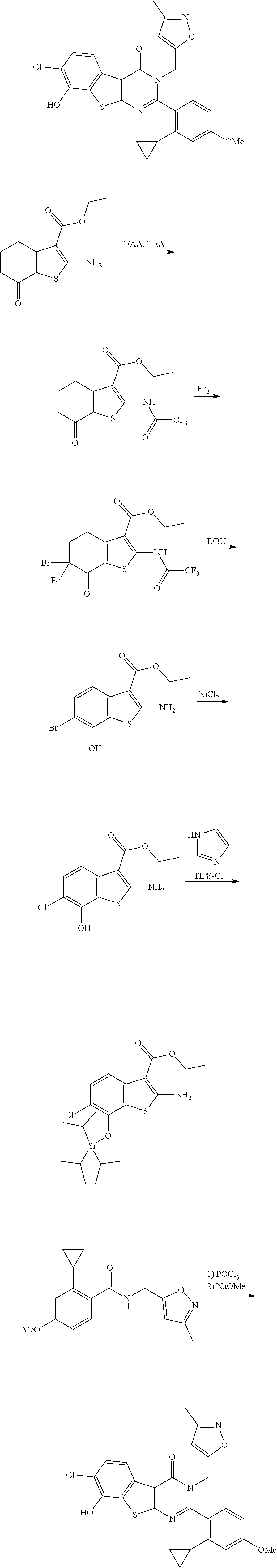

To a stirred solution of ethyl 2-amino-7-oxo-4,5,6,7-tetrahydrobenzo[b]thiophene-3-carboxylate (15.0 g, 62.7 mmol) and TEA (12.7 g, 17.5 mL, 125 mmol) in DCM (179 mL) was slowly added TFAA (18.4 g, 12.4 mL, 87.8 mmol) at rt. The mixture was allowed to stir at rt for 12 h. After the reaction was complete, the mixture was evaporated to dryness and diluted with EA (200 mL), and then quickly washed with 10% citric acid twice and brine once. The organic layer was dried and concentrated in vacuo to afford ethyl 7-oxo-2-(2,2,2-trifluoroacetamido)-4,5,6,7-tetrahydrobenzo[b]thiophene-3-carboxylate 21 g, as a solid and used directly in the next step. To a 500 mL round-bottomed flask was charged ethyl 7-oxo-2-(2,2,2-trifluoroacetamido)-4,5,6,7-tetrahydrobenzo[b]thiophene-3-carboxylate (21 g, 63 mmol) and DCM (0.21 L) and followed by the addition of dibromine (24 g, 7.7 mL, 0.15 mol) slowly. The reaction was stirred at rt overnight. Quenched by the addition of sat. Na₂S₂O₃ and diluted with DCM. The organic layer was separated and evaporated to dryness. Dried in vacuo to give ethyl 6,6-dibromo-7-oxo-2-(2,2,2-trifluoroacetamido)-4,5,6,7-tetrahydrobenzo[b]thiophene-3-carboxylate, 28 g. Used directly without purification.

To a 1 L round-bottomed flask was charged ethyl 6,6-dibromo-7-oxo-2-(2,2,2-trifluoroacetamido)-4,5,6,7-tetrahydrobenzo[b]thiophene-3-carboxylate (28.0 g, 56.8 mmol) and ACN (227 mL). Then, DBU (51.9 g, 51.4 mL, 341 mmol) was added slowly to the mixture at 0° C. The reaction was moved to rt and stirred for 30 mins. Water (115 mL) was charged to the mixture, followed by the addition of potassium carbonate (11.8 g, 85.2 mmol). The mixture was stirred at rt for overnight. ACN was evaporated under vacuum and the remaining aqueous layer was extracted with EA. The combined organic layer was washed with sat. NH₄Cl, sat. NaHCO₃, and brine respectively. The organic layer was dried, filtered and concentrated to dryness. DCM (70 mL) was added and the slurry was stirred at rt overnight. Filtered and dried to give a white solid 5.35 g. The mother liquid was purified by column Hexane to 30% EA/Hexane to give 6.65 g of product. The materials were combined to give ethyl 2-amino-6-bromo-7-hydroxybenzo[b]thiophene-3-carboxylate, 12 g.

To a 25 mL microwave vial was charged ethyl 2-amino-6-bromo-7-hydroxybenzo[b]thiophene-3-carboxylate (1 g, 3.16 mmol), nickel dichloride (2.87 g, 22.1 mmol), and DMF (15 mL). The mixture was irradiated at 170° C. under microwave for 25 min. Another 3 runs were carried out under identical condition. Upon completion, the mixture was combined, diluted with EtOAc and water. The organic layer was separated and washed with water thrice. The organic layer was then concentrated in vacuo to afford compound ethyl 2-amino-6-chloro-7-hydroxybenzo[b]thiophene-3-carboxylate as a white solid (3.43 g, quan. yield). LC-MS observed [M+H], 271.92. To a 250 mL round-bottomed flask was charged with ethyl 2-amino-6-chloro-7-hydroxybenzo[b]thiophene-3-carboxylate (3.43 g, 12.6 mmol), imidazole (4.30 g, 63.1 mmol) and DMF (42.1 mL) respectively followed by dropwise addition of TIPS-Cl (6.08 g, 6.75 mL, 31.6 mmol). The mixture was heated at 80° C. for 18 h. Diluted with 250 mL of EA, and the organic mixture was washed with water. Aqueous layer extracted by ethyl acetate once. The combined organic layers were washed with water (×2) and brine (×2). Dried, filtered, concentrated and purified by CombiFlash to give ethyl 2-amino-6-chloro-7-((triisopropylsilyl)oxy)benzo[b]thiophene-3-carboxylate as a white solid, 3.73 g, 69% yield. LC-MS observed [M+H], 428.17.

To a 2-dram vial were added ethyl 2-amino-6-chloro-7-((triisopropylsilyl)oxy)benzo[b]thiophene-3-carboxylate (112 mg, 263 μmol), 2-cyclopropyl-4-methoxy-N-((3-methylisoxazol-5-yl)methyl)benzamide (75.2 mg, 263 μmol) (the synthesis was reported at WO2023/212019), DCE (2.63 mL) followed by addition of phosphoryl trichloride (52.3 mg, 31.8 μL, 341 μmol). The solution was stirred at 85° C. overnight. Sodium methanolate (0.14 g, 5.0 mL, 0.5 M, 2.5 mmol) was added and the rxn mixture was stirred at 65° C. for 2 h. Concentrated to remove solvents. MeOH (2.5 mL) was added and the mixture was stirred at 65° C. for 45 min. RXN was complete by LC/MS. Cooled to rt. Diluted with DCM, washed with 10% citric acid, Sat. NaHCO₃ and brine respectively. Dried, filtered, concentrated, and purified by CombiFlash (12 g SiO₂, Ace/Cyclohexane: 0˜50%) to give 7-chloro-2-(2-cyclopropyl-4-methoxyphenyl)-8-hydroxy-3-((3-methylisoxazol-5-yl)methyl)benzo[4,5]thieno[2,3-d]pyrimidin-4(3H)-one (101.3 mg, 78.1%) as a white solid. LC-MS observed [M+H], 494.09. ¹H NMR (400 MHz, CDCl₃) δ 8.16 (d, J=8.5 Hz, 1H), 7.47 (d, J=8.5 Hz, 1H), 7.28 (d, J=8.5 Hz, 1H), 6.80 (dd, J=8.5, 2.5 Hz, 1H), 6.59 (s, 1H), 6.47 (d, J=2.5 Hz, 1H), 6.03 (s, 1H), 5.59 (d, J=15.5 Hz, 1H), 5.03 (d, J=15.5 Hz, 1H), 3.82 (s, 3H), 2.22 (s, 3H), 1.66-1.56 (m, 1H), 0.96-0.77 (m, 3H), 0.70-0.59 (m, 1H).

### Synthesis of 7-chloro-2-(2-cyclopropyl-4-fluorophenyl)-8-hydroxy-3-((3-(trifluoromethyl)isoxazol-5-yl)methyl)benzo[4,5]thieno[2,3-d]pyrimidin-4(3H)-one (example 40)

### Example 40

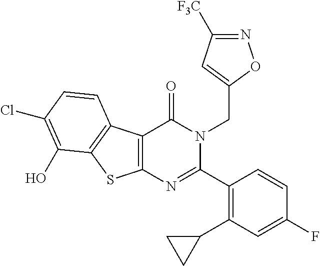 [SMILES: O=c1c2c(nc(-c3ccc(F)cc3C3CC3)n1Cc1cc(C(F)(F)F)no1)sc1c(O)c(Cl)ccc12]

Example 40 was prepared using a procedure analogous to that described for example 39.

### Synthesis of 7-chloro-2-(2-cyclopropyl-4-methoxyphenyl)-3-((2-ethylthiazol-5-yl)methyl)-8-hydroxybenzo[4,5]thieno[2,3-d]pyrimidin-4(3H)-one (example 41)

### Example 41

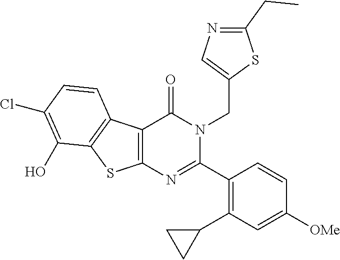 [SMILES: CCc1ncc(Cn2c(-c3ccc(OC)cc3C3CC3)nc3sc4c(O)c(Cl)ccc4c3c2=O)s1]

Example 41 was prepared using a procedure analogous to that described for example 39.

### Synthesis of 7-chloro-2-(2-cyclopropyl-4-fluorophenyl)-8-hydroxy-3-(oxazol-5-ylmethyl)benzo[4,5]thieno[2,3-d]pyrimidin-4(3H)-one (example 42)

### Example 42

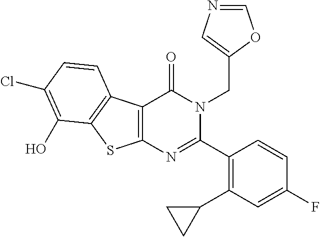 [SMILES: O=c1c2c(nc(-c3ccc(F)cc3C3CC3)n1Cc1cnco1)sc1c(O)c(Cl)ccc12]

Example 40 was prepared using a procedure analogous to that described for example 39.

### Synthesis of 7-chloro-2-(2-cyclopropyl-4-fluorophenyl)-8-hydroxy-3-((3-methylisoxazol-5-yl)methyl)benzo[4,5]thieno[2,3-d]pyrimidin-4(3H)-one (example 43)

### Example 43

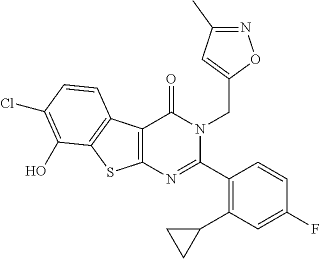 [SMILES: Cc1cc(Cn2c(-c3ccc(F)cc3C3CC3)nc3sc4c(O)c(Cl)ccc4c3c2=O)on1]

Example 43 was prepared using a procedure analogous to that described for example 39.

### Synthesis of 7-chloro-2-(2-cyclopropyl-4-fluorophenyl)-8-hydroxy-3-((2-methyloxazol-5-yl)methyl)benzo[4,5]thieno[2,3-d]pyrimidin-4(3H)-one (example 44)

### Example 44

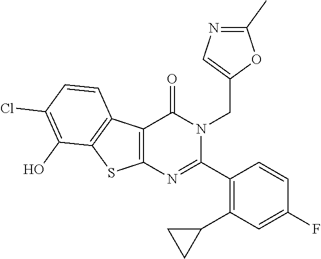 [SMILES: Cc1ncc(Cn2c(-c3ccc(F)cc3C3CC3)nc3sc4c(O)c(Cl)ccc4c3c2=O)o1]

Example 44 was prepared using a procedure analogous to that described for example 39.

### Synthesis of methyl 2-(7-chloro-2-(2-cyclopropyl-4-methoxyphenyl)-8-hydroxy-4-oxobenzo[4,5]thieno[2,3-d]pyrimidin-3(4H)-yl)acetate (example 45)

### Example 45

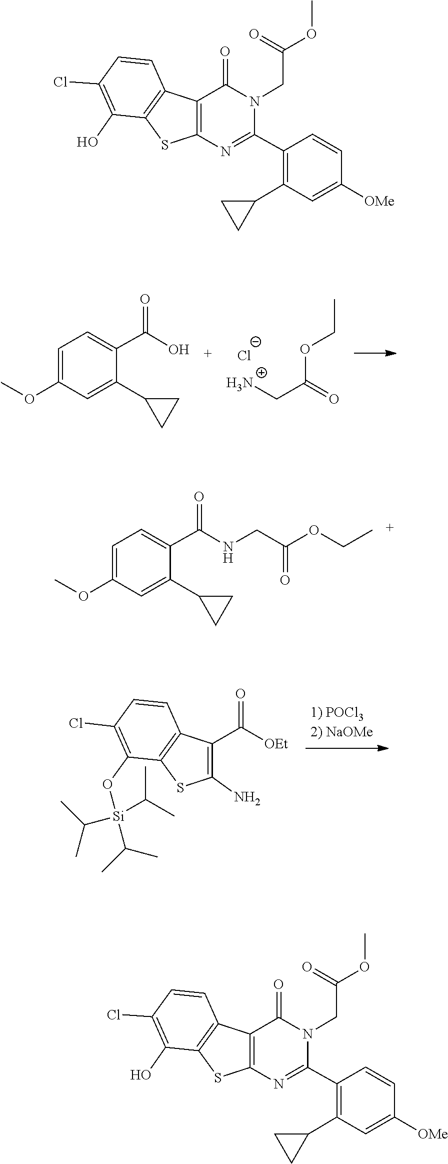

To a 250 mL round-bottomed flask were added 2-cyclopropyl-4-methoxybenzoic acid (3.00 g, 15.6 mmol) and thionyl chloride (5.57 g, 3.42 mL, 46.8 mmol) and the black solution were stirred at 85° C. for 50 min. Co-evaporated with toluene once to give 2-cyclopropyl-4-methoxybenzoyl chloride (3.45 g, quan. yield). Dissolved in DCM (20.8 mL). To a 250 mL round-bottomed flask were added H-Gly-OEt Hydrochloride (2.18 g, 15.6 mmol), and DCM (83.2 mL), and the suspension was cooled to 0° C. followed by addition of triethylamine (6.32 g, 8.70 mL, 62.4 mmol) dropwise to give a white suspension. After stirring for 0.5 h at 0° C., the solution of 2-cyclopropyl-4-methoxybenzoyl chloride (3.45 g, 16.4 mmol) was added dropwise over 15 min to give a brown suspension. The mixture was stirred at 0° C.˜rt for 20 h. Diluted with DCM, washed with water twice and then brine once. Dried, filtered, concentrated and purified by CombiFlash (120 g SiO₂, Ace/c-Hex: 0˜50%) to give methyl 2-(7-chloro-2-(2-cyclopropyl-4-methoxyphenyl)-8-hydroxy-4-oxobenzo[4,5]thieno[2,3-d]pyrimidin-3(4H)-yl)acetate (3.36 g, 77.6%) as an off-white solid.

### Synthesis of 2-(7-chloro-2-(2-cyclopropyl-4-methoxyphenyl)-8-hydroxy-4-oxobenzo[4,5]thieno[2,3-d]pyrimidin-3(4H)-yl)acetic acid (example 46)

### Example 46

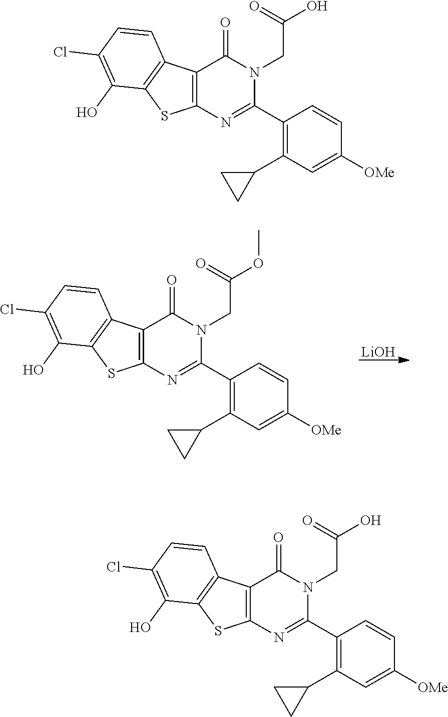

To a 2-dram vial were added methyl 2-(7-chloro-2-(2-cyclopropyl-4-methoxyphenyl)-8-hydroxy-4-oxobenzo[4,5]thieno[2,3-d]pyrimidin-3(4H)-yl)acetate (80 mg, 0.17 mmol), THF (1.15 mL), and a solution of LiOH (41 mg, 1.7 mmol) in H₂O (2.30 mL) and the mixture was stirred at rt for 2.5 h. Diluted with DCM, washed with 10% citric acid and brine. Dried, filtered, concentrated to give 2-(7-chloro-2-(2-cyclopropyl-4-methoxyphenyl)-8-hydroxy-4-oxobenzo[4,5]thieno[2,3-d]pyrimidin-3(4H)-yl)acetic acid (78 mg, 100%) as an off-white solid.

### Synthesis of 7-chloro-2-(2-cyclopropyl-4-methoxyphenyl)-8-hydroxy-3-(isoxazol-5-ylmethyl)benzo[4,5]thieno[2,3-d]pyrimidin-4(3H)-one (example 47)

### Example 47

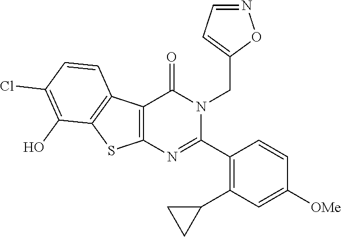 [SMILES: COc1ccc(-c2nc3sc4c(O)c(Cl)ccc4c3c(=O)n2Cc2ccno2)c(C2CC2)c1]

Example 47 was prepared using a procedure analogous to that described for example 39.

### Synthesis of 7-chloro-2-(2-cyclopropyl-4-methoxyphenyl)-8-hydroxy-3-((3-(trifluoromethyl)isoxazol-5-yl)methyl)benzo[4,5]thieno[2,3-d]pyrimidin-4(3H)-one (example 48)

### Example 48

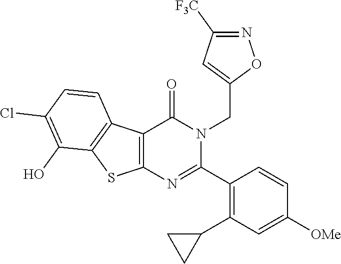 [SMILES: COc1ccc(-c2nc3sc4c(O)c(Cl)ccc4c3c(=O)n2Cc2cc(C(F)(F)F)no2)c(C2CC2)c1]

Example 48 was prepared using a procedure analogous to that described for example 39.

### Synthesis of 7-chloro-2-(2-cyclopropyl-4-methoxyphenyl)-3-((2-cyclopropyloxazol-5-yl)methyl)-8-hydroxybenzo[4,5]thieno[2,3-d]pyrimidin-4(3H)-one (example 49)

### Example 49

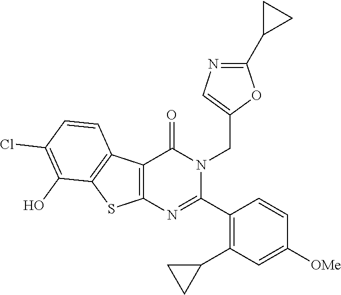 [SMILES: COc1ccc(-c2nc3sc4c(O)c(Cl)ccc4c3c(=O)n2Cc2cnc(C3CC3)o2)c(C2CC2)c1]

Example 49 was prepared using a procedure analogous to that described for example 39.

### Synthesis of 7-chloro-2-(2-cyclopropyl-4-fluorophenyl)-3-((2-cyclopropyloxazol-5-yl)methyl)-8-hydroxybenzo[4,5]thieno[2,3-d]pyrimidin-4(3H)-one (example 50)

### Example 50

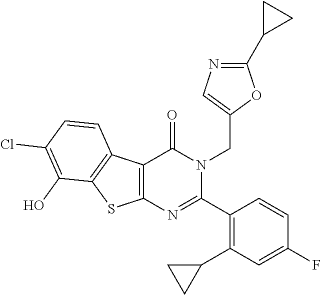 [SMILES: O=c1c2c(nc(-c3ccc(F)cc3C3CC3)n1Cc1cnc(C3CC3)o1)sc1c(O)c(Cl)ccc12]

Example 50 was prepared using a procedure analogous to that described for example 39.

### Synthesis of 2-(7-chloro-2-(2-cyclopropyl-4-methoxyphenyl)-8-hydroxy-4-oxobenzo[4,5]thieno[2,3-d]pyrimidin-3(4H)-yl)-N-(2-hydroxyethyl)acetamide (example 51)

### Example 51

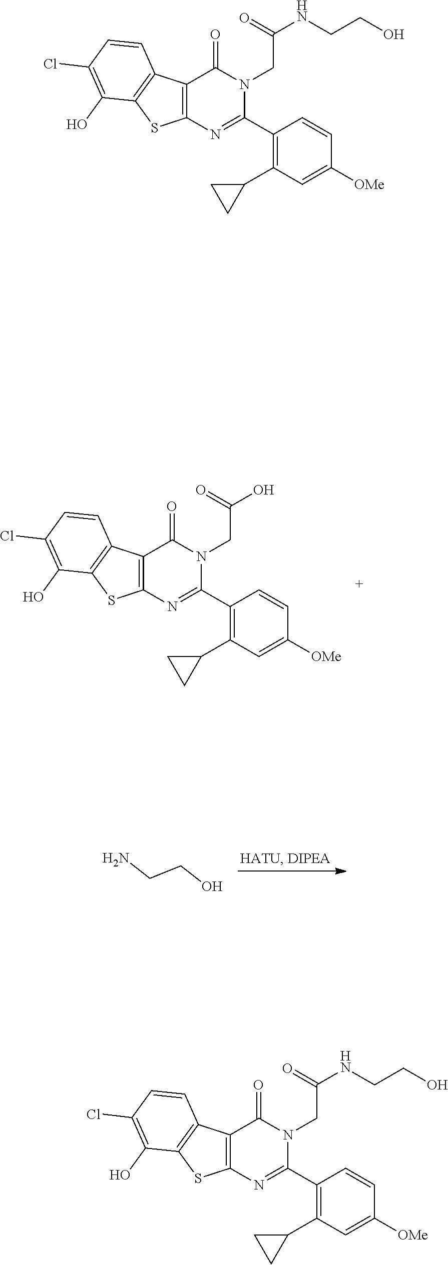

To a 1-dram vial were added 2-(7-chloro-2-(2-cyclopropyl-4-methoxyphenyl)-8-hydroxy-4-oxobenzo[4,5]thieno[2,3-d]pyrimidin-3(4H)-yl)acetic acid (80.0 mg, 175.1 μmol), 2-aminoethan-1-ol (23.28 mg, 23.00 μL, 381.1 μmol), HATU (86.55 mg, 227.6 μmol), DCM (1 mL), DMF (1 mL), and DIPEA (67.89 mg, 92 μL, 525.3 μmol) respectively and the resulting yellow solution was stirred at rt overnight. Diluted with DCM, washed with 10% citric acid, sat. NaHCO₃ and brine. Dried, filtered, concentrated and purified by CombiFlash (12 g SiO₂, EA/c-Hex: 0˜100%) to give 2-(7-chloro-2-(2-cyclopropyl-4-methoxyphenyl)-8-hydroxy-4-oxobenzo[4,5]thieno[2,3-d]pyrimidin-3(4H)-yl)-N-(2-hydroxyethyl)acetamide (40 mg, 46%) as a white solid.

### Synthesis of 7-chloro-2-(2-cyclopropyl-4-fluorophenyl)-8-hydroxy-3-(isoxazol-5-ylmethyl)benzo[4,5]thieno[2,3-d]pyrimidin-4(3H)-one (example 52)

### Example 52

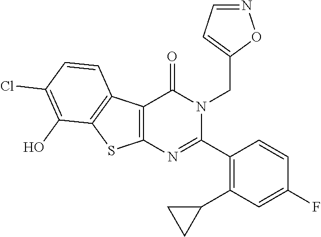 [SMILES: O=c1c2c(nc(-c3ccc(F)cc3C3CC3)n1Cc1ccno1)sc1c(O)c(Cl)ccc12]

Example 40 was prepared using a procedure analogous to that described for example 39.

### Synthesis of 7-chloro-2-(2-cyclopropyl-4-methoxyphenyl)-3-((2,4-dimethylthiazol-5-yl)methyl)-8-hydroxybenzo[4,5]thieno[2,3-d]pyrimidin-4(3H)-one (example 53)

### Example 53

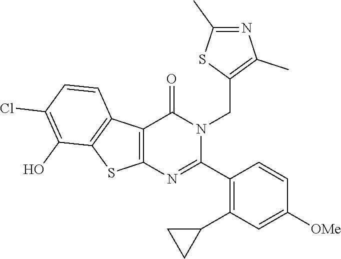 [SMILES: COc1ccc(-c2nc3sc4c(O)c(Cl)ccc4c3c(=O)n2Cc2sc(C)nc2C)c(C2CC2)c1]

Example 53 was prepared using a procedure analogous to that described for example 39.

### Synthesis of 7-chloro-2-(2-cyclopropyl-4-methoxyphenyl)-3-((4,5-dihydrooxazol-2-yl)methyl)-8-hydroxybenzo[4,5]thieno[2,3-d]pyrimidin-4(3H)-one (example 54)

### Example 54

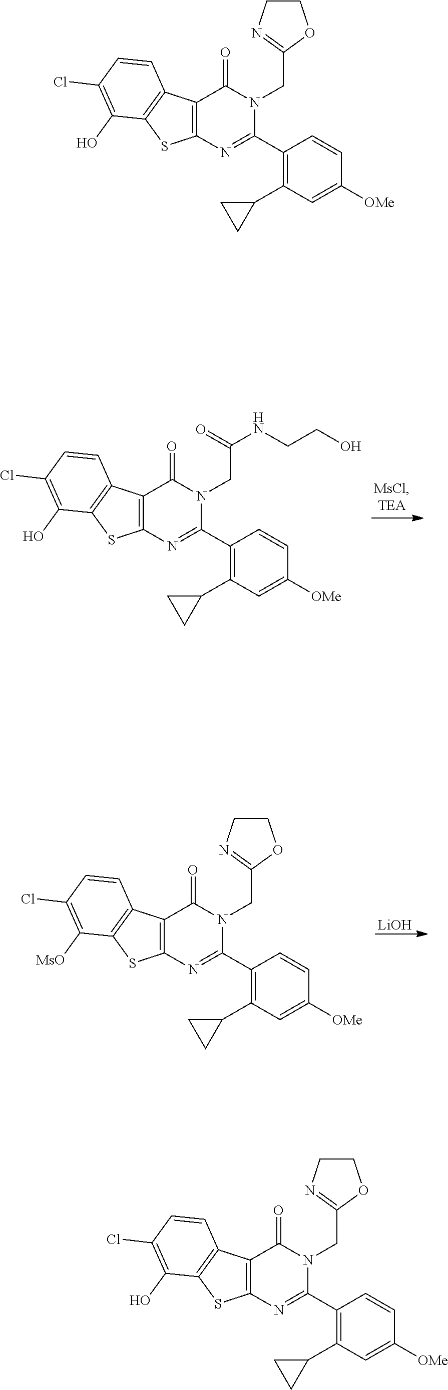 [SMILES: COc1ccc(-c2nc3sc4c(O)c(Cl)ccc4c3c(=O)n2CC2=NCCO2)c(C2CC2)c1 ; COc1ccc(-c2nc3sc4c(O)c(Cl)ccc4c3c(=O)n2CC(=O)NCCO)c(C2CC2)c1 ; COc1ccc(-c2nc3sc4c(OS(C)(=O)=O)c(Cl)ccc4c3c(=O)n2CC2=NCCO2)c(C2CC2)c1 ; "LiOH" ; COc1ccc(-c2nc3sc4c(O)c(Cl)ccc4c3c(=O)n2CC2=NCCO2)c(C2CC2)c1]

To a 2-dram vial were added 2-(7-chloro-2-(2-cyclopropyl-4-methoxyphenyl)-8-hydroxy-4-oxobenzo[4,5]thieno[2,3-d]pyrimidin-3(4H)-yl)-N-(2-hydroxyethyl)acetamide (38 mg, 76 μmol), DCM (2 mL) and TEA (38 mg, 53 μL, 0.38 mmol) respectively and followed by addition of mesyl chloride (26 mg, 18 μL, 0.23 mmol) dropwise. The solution was stirred at 40° C. for 30 h. Cooled to rt. Diluted with DCM, washed with Sat. NaHCO₃ and brine respectively. Dried, filtered, and concentrated to give 7-chloro-2-(2-cyclopropyl-4-methoxyphenyl)-3-((4,5-dihydrooxazol-2-yl)methyl)-4-oxo-3,4-dihydrobenzo[4,5]thieno[2,3-d]pyrimidin-8-yl methanesulfonate (47 mg, quan. yield) as a pale yellow solid. LC-MS observed [M+H], 559.78. Used directly without further purification.

To a 1-dram vial were added 7-chloro-2-(2-cyclopropyl-4-methoxyphenyl)-3-((4,5-dihydrooxazol-2-yl)methyl)-4-oxo-3,4-dihydrobenzo[4,5]thieno[2,3-d]pyrimidin-8-yl methanesulfonate (10.9 mg, 19.5 μmol), THF (1.2 mL), and a solution of LiOH (9.32 mg, 389 mol) in water (0.60 mL) respectively and the rxn was stirred at rt for 2 h. Diluted with DCM, washed with 10% citric acid and Sat. NaHCO₃ respectively. Dried, filtered, concentrated and purified by CombiFlash (12 SiO₂, Ace/c-Hex: 0˜40%) to give 7-chloro-2-(2-cyclopropyl-4-methoxyphenyl)-3-((4,5-dihydrooxazol-2-yl)methyl)-8-hydroxybenzo[4,5]thieno[2,3-d]pyrimidin-4(3H)-one (7.00 mg, 74.6%) as a white solid.

### Synthesis of 2-(2-cyclopropyl-4-methoxyphenyl)-5-fluoro-8-hydroxy-3-(oxazol-5-ylmethyl)benzo[4,5]thieno[2,3-d]pyrimidin-4(3H)-one (example 55) and 2-(2-cyclopropyl-4-methoxyphenyl)-7-fluoro-8-hydroxy-3-(oxazol-5-ylmethyl)benzo[4,5]thieno[2,3-d]pyrimidin-4(3H)-one (example 56)

### Examples 55 and 56

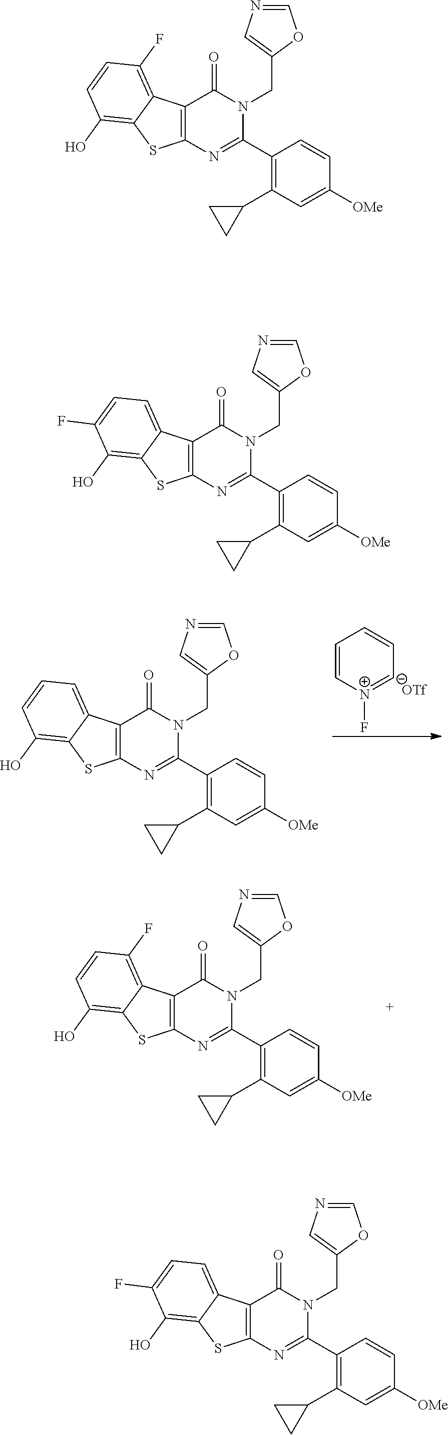 [SMILES: COc1ccc(-c2nc3sc4c(O)ccc(F)c4c3c(=O)n2Cc2cnco2)c(C2CC2)c1 ; COc1ccc(-c2nc3sc4c(O)c(F)ccc4c3c(=O)n2Cc2cnco2)c(C2CC2)c1 ; COc1ccc(-c2nc3sc4c(O)cccc4c3c(=O)n2Cc2cnco2)c(C2CC2)c1 ; F[n+]1[c-]cccc1 ; COc1ccc(-c2nc3sc4c(O)ccc(F)c4c3c(=O)n2Cc2cnco2)c(C2CC2)c1 ; COc1ccc(-c2nc3sc4c(O)c(F)ccc4c3c(=O)n2Cc2cnco2)c(C2CC2)c1]

To a 40 mL vial were added 2-(2-cyclopropyl-4-methoxyphenyl)-8-hydroxy-3-(oxazol-5-ylmethyl)benzo[4,5]thieno[2,3-d]pyrimidin-4(3H)-one (200 mg, 449 μmol) (the synthesis was reported at WO2023/212019), 1-fluoropyridin-1-ium trifluoromethanesulfonate (277 mg, 1.12 mmol), and ACN (4.00 mL) respectively and the suspension was stirred at 80° C. for 15 h. After cooled to rt, stirred with Sat. Na₂S₂SO₃ solution for a few hours. Extracted with DCM, washed with Sat. NaHCO₃ and brine. The organic layer was dried (Na₂SO₄). Filtered, concentrated, and purified by CombiFlash (12 g SiO₂, Ace/c-Hex: 0˜40%) and then purified by Prep TLC (40% Ace/c-Hex) to give 2-(2-cyclopropyl-4-methoxyphenyl)-5-fluoro-8-hydroxy-3-(oxazol-5-ylmethyl)benzo[4,5]thieno[2,3-d]pyrimidin-4(3H)-one (5.5 mg, 2.6% yield) and 2-(2-cyclopropyl-4-methoxyphenyl)-7-fluoro-8-hydroxy-3-(oxazol-5-ylmethyl)benzo[4,5]thieno[2,3-d]pyrimidin-4(3H)-one (2.65 mg, 1.2% yield).

### Synthesis of 7-chloro-2-(2-cyclopropyl-4-methoxyphenyl)-8-hydroxy-3-(isoxazol-4-ylmethyl)benzo[4,5]thieno[2,3-d]pyrimidin-4(3H)-one (example 57)

### Example 57

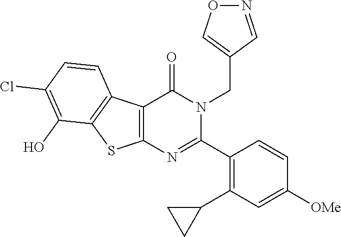 [SMILES: COc1ccc(-c2nc3sc4c(O)c(Cl)ccc4c3c(=O)n2Cc2cnoc2)c(C2CC2)c1]

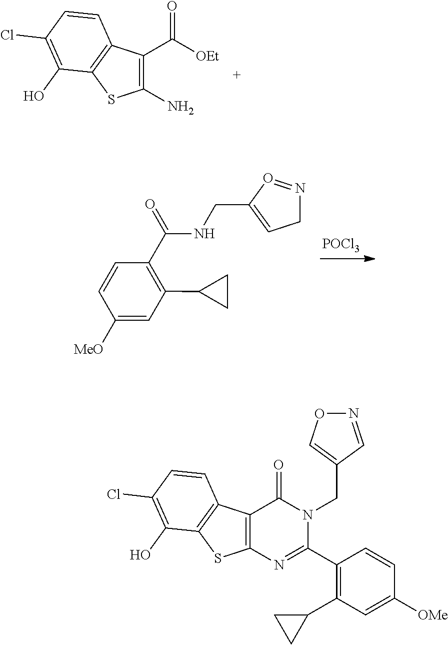

To a 1-dram vial were added ethyl 2-amino-6-chloro-7-hydroxybenzo[b]thiophene-3-carboxylate (30 mg, 0.11 mmol), 2-cyclopropyl-N-(isoxazol-4-ylmethyl)-4-methoxybenzamide (30 mg, 0.11 mmol), DCE (1.0 mL) followed by addition of phosphoryl trichloride (22 mg, 13 μL, 0.14 mmol). The solution was stirred at 80° C. overnight. Diluted with a few mL of DCM, stirred with ˜2 mL of Sat. NaHCO₃ at rt for 1 h. Diluted with DCM, washed with Sat. NaHCO₃ and brine respectively. Dried (Na₂SO₄), filtered, concentrated, and purified by Prep HPLC (20˜90% ACN, 0.1% TFA) to give 7-chloro-2-(2-cyclopropyl-4-methoxyphenyl)-8-hydroxy-3-(isoxazol-4-ylmethyl)benzo[4,5]thieno[2,3-d]pyrimidin-4(3H)-one (23.5 mg, 44%).

### Synthesis of 7-chloro-2-(2-cyclopropyl-4-methoxyphenyl)-8-hydroxy-3-((2-methylpyrimidin-5-yl)methyl)benzo[4,5]thieno[2,3-d]pyrimidin-4(3H)-one (example 58)

### Example 58

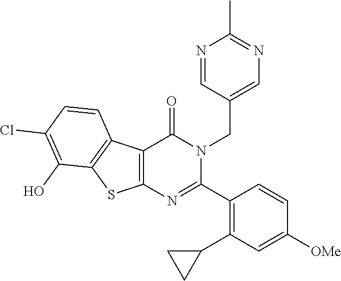 [SMILES: COc1ccc(-c2nc3sc4c(O)c(Cl)ccc4c3c(=O)n2Cc2cnc(C)nc2)c(C2CC2)c1]

Example 58 was prepared using a procedure analogous to that described for example 57.

### Synthesis of 7-chloro-2-(2-cyclopropyl-4-methoxyphenyl)-8-hydroxy-3-((2-methyloxazol-5-yl)methyl)benzo[4,5]thieno[2,3-d]pyrimidin-4(3H)-one (example 59)

### Example 59

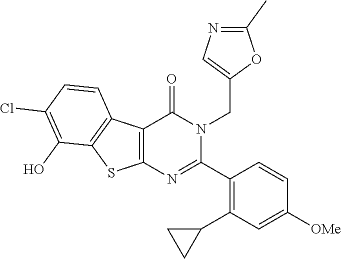 [SMILES: COc1ccc(-c2nc3sc4c(O)c(Cl)ccc4c3c(=O)n2Cc2cnc(C)o2)c(C2CC2)c1]

Example 59 was prepared using a procedure analogous to that described for example 39.

### Synthesis of 7-chloro-2-(2-cyclopropyl-4,5-difluorophenyl)-8-hydroxy-3-((3-methylisoxazol-5-yl)methyl)benzo[4,5]thieno[2,3-d]pyrimidin-4(3H)-one (example 60)

### Example 60

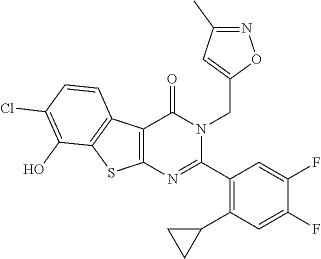 [SMILES: Cc1cc(Cn2c(-c3cc(F)c(F)cc3C3CC3)nc3sc4c(O)c(Cl)ccc4c3c2=O)on1]

Example 60 was prepared using a procedure analogous to that described for example 39.

### Synthesis of 7-chloro-2-(2-cyclopropyl-4,5-difluorophenyl)-8-hydroxy-3-((2-methyloxazol-5-yl)methyl)benzo[4,5]thieno[2,3-d]pyrimidin-4(3H)-one (example 61)

### Example 61

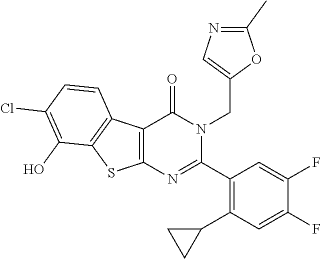 [SMILES: Cc1ncc(Cn2c(-c3cc(F)c(F)cc3C3CC3)nc3sc4c(O)c(Cl)ccc4c3c2=O)o1]

Example 61 was prepared using a procedure analogous to that described for example 39.

### Synthesis of 7-chloro-2-(2-cyclopropyl-4,5-difluorophenyl)-8-hydroxy-3-(isoxazol-5-ylmethyl)benzo[4,5]thieno[2,3-d]pyrimidin-4(3H)-one (example 62)

### Example 62

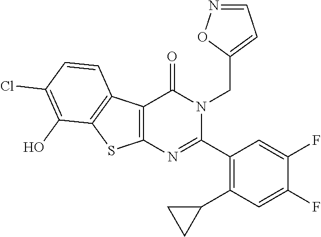 [SMILES: O=c1c2c(nc(-c3cc(F)c(F)cc3C3CC3)n1Cc1ccno1)sc1c(O)c(Cl)ccc12]

Example 62 was prepared using a procedure analogous to that described for example 57.

### Synthesis of 7-chloro-2-(2-cyclopropyl-4,5-difluorophenyl)-8-hydroxy-3-((2-(trifluoromethyl)pyrimidin-5-yl)methyl)benzo[4,5]thieno[2,3-d]pyrimidin-4(3H)-one (example 63)

### Example 63

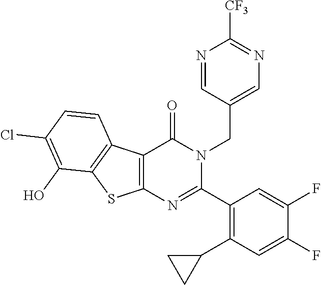 [SMILES: O=c1c2c(nc(-c3cc(F)c(F)cc3C3CC3)n1Cc1cnc(C(F)(F)F)nc1)sc1c(O)c(Cl)ccc12]

Example 63 was prepared using a procedure analogous to that described for example 39.

### Synthesis of 7-chloro-2-(2-cyclopropyl-4-(methylsulfonyl)phenyl)-8-hydroxy-3-((2-(trifluoromethyl)pyrimidin-5-yl)methyl)benzo[4,5]thieno[2,3-d]pyrimidin-4(3H)-one (example 64)

### Example 64

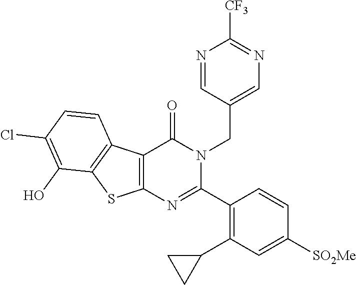 [SMILES: CS(=O)(=O)c1ccc(-c2nc3sc4c(O)c(Cl)ccc4c3c(=O)n2Cc2cnc(C(F)(F)F)nc2)c(C2CC2)c1]

Example 64 was prepared using a procedure analogous to that described for example 39.

### Synthesis of 7-chloro-2-(2-cyclopropyl-4,5-difluorophenyl)-3-((3-(difluoromethyl)isoxazol-5-yl)methyl)-8-hydroxybenzo[4,5]thieno[2,3-d]pyrimidin-4(3H)-one (example 65)

### Example 65

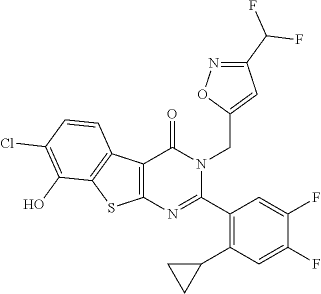 [SMILES: O=c1c2c(nc(-c3cc(F)c(F)cc3C3CC3)n1Cc1cc(C(F)F)no1)sc1c(O)c(Cl)ccc12]

Example 65 was prepared using a procedure analogous to that described for example 39.

### Synthesis of 7-chloro-2-(2-cyclopropyl-4,5-difluorophenyl)-8-hydroxy-3-((5-(trifluoromethyl)isoxazol-3-yl)methyl)benzo[4,5]thieno[2,3-d]pyrimidin-4(3H)-one (example 66)

### Example 66

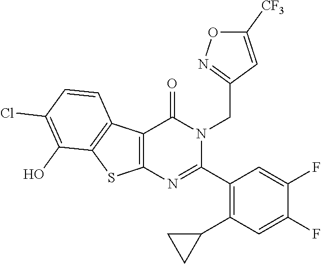 [SMILES: O=c1c2c(nc(-c3cc(F)c(F)cc3C3CC3)n1Cc1cc(C(F)(F)F)on1)sc1c(O)c(Cl)ccc12]

Example 66 was prepared using a procedure analogous to that described for example 39.

### Synthesis of 7-chloro-2-(2-cyclopropyl-6-(trifluoromethyl)pyridin-3-yl)-8-hydroxy-3-((2-(trifluoromethyl)pyrimidin-5-yl)methyl)benzo[4,5]thieno[2,3-d]pyrimidin-4(3H)-one (example 67)

### Example 67

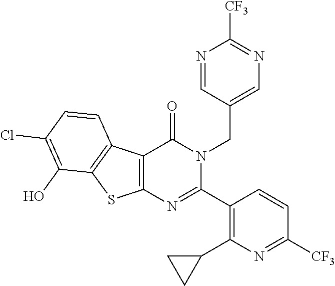 [SMILES: O=c1c2c(nc(-c3ccc(C(F)(F)F)nc3C3CC3)n1Cc1cnc(C(F)(F)F)nc1)sc1c(O)c(Cl)ccc12]

Example 67 was prepared using a procedure analogous to that described for example 39.

### Synthesis of 7-chloro-2-(2-cyclopropyl-6-(trifluoromethyl)pyridin-3-yl)-8-hydroxy-3-((6-(trifluoromethyl)pyridin-3-yl)methyl)benzo[4,5]thieno[2,3-d]pyrimidin-4(3H)-one (example 68)

### Example 68

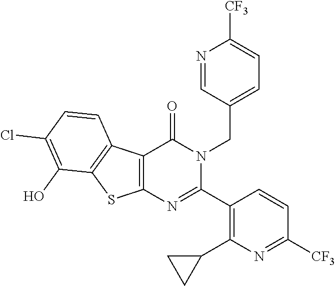 [SMILES: O=c1c2c(nc(-c3ccc(C(F)(F)F)nc3C3CC3)n1Cc1ccc(C(F)(F)F)nc1)sc1c(O)c(Cl)ccc12]

Example 68 was prepared using a procedure analogous to that described for example 39.

### Synthesis of 7-chloro-2-(2-cyclopropyl-4-(methylsulfonyl)phenyl)-8-hydroxy-3-((6-(trifluoromethyl)pyridin-3-yl)methyl)benzo[4,5]thieno[2,3-d]pyrimidin-4(3H)-one (example 69)

### Example 69

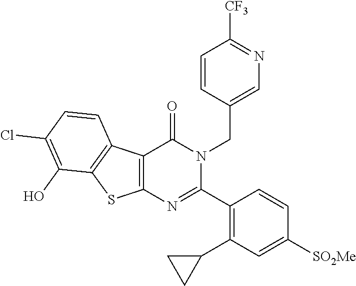 [SMILES: CS(=O)(=O)c1ccc(-c2nc3sc4c(O)c(Cl)ccc4c3c(=O)n2Cc2ccc(C(F)(F)F)nc2)c(C2CC2)c1]

Example 69 was prepared using a procedure analogous to that described for example 39.

### Synthesis of 7-chloro-2-(2-cyclopropyl-4-(methylsulfonyl)phenyl)-8-hydroxy-3-((1-methyl-6-oxo-1,6-dihydropyridin-3-yl)methyl)benzo[4,5]thieno[2,3-d]pyrimidin-4(3H)-one (example 70)

### Example 70

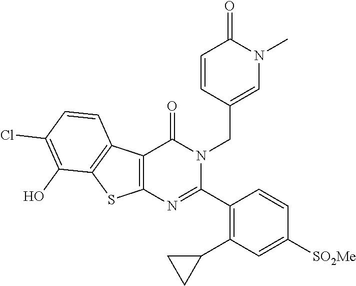 [SMILES: Cn1cc(Cn2c(-c3ccc(S(C)(=O)=O)cc3C3CC3)nc3sc4c(O)c(Cl)ccc4c3c2=O)ccc1=O]

Example 70 was prepared using a procedure analogous to that described for example 39.

### Synthesis of 2-(2-cyclopropyl-4-(methylsulfonyl)phenyl)-7-fluoro-8-hydroxy-3-((2-(trifluoromethyl)pyrimidin-5-yl)methyl)benzo[4,5]thieno[2,3-d]pyrimidin-4(3H)-one (example 71)

### Example 71

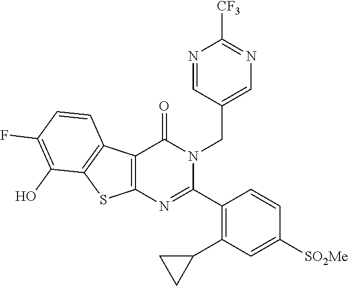 [SMILES: CS(=O)(=O)c1ccc(-c2nc3sc4c(O)c(F)ccc4c3c(=O)n2Cc2cnc(C(F)(F)F)nc2)c(C2CC2)c1]

Example 71 was prepared using a procedure analogous to that described for example 56.

### Synthesis of (7-chloro-2-(2-cyclopropyl-4-ethoxyphenyl)-8-hydroxy-3-(oxazol-5-ylmethyl)benzo[4,5]thieno[2,3-d]pyrimidin-4(3H)-one) (example 72)

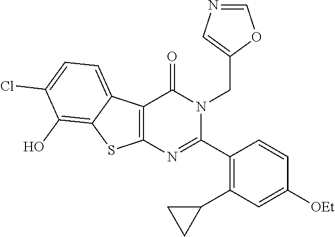 [SMILES: CCOc1ccc(-c2nc3sc4c(O)c(Cl)ccc4c3c(=O)n2Cc2cnco2)c(C2CC2)c1]

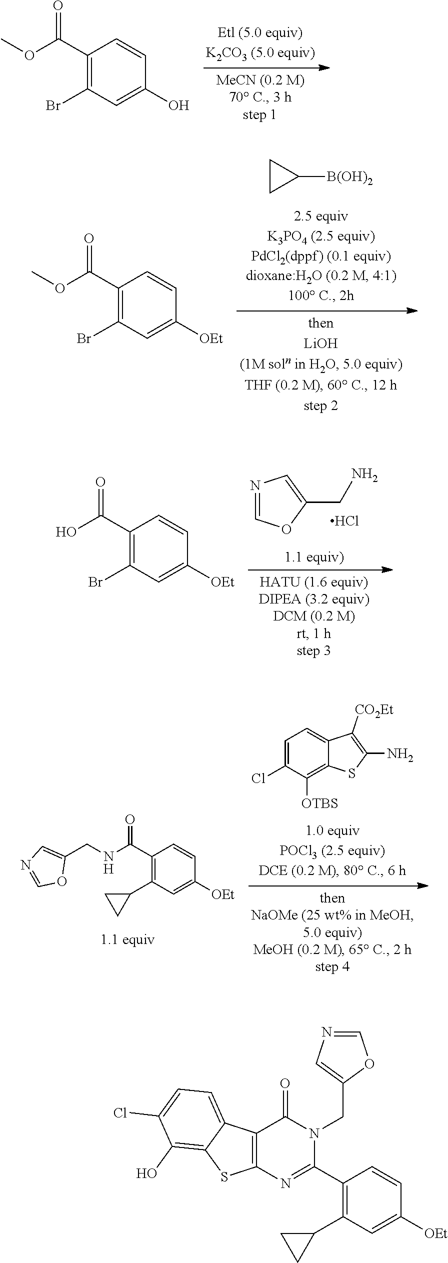

### Step 1

In a 40 mL vial equipped with a stir bar, methyl 2-bromo-4-hydroxybenzoate (500 mg, 1.0 equiv., CAS #101085-03-4) was dissolved in dry acetonitrile (10.8 mL) at room temperature, followed by added potassium carbonate (1.5 g, 5.0 equiv.). Subsequently, iodoethane (0.87 mL, 5.0 equiv.) was added dropwise and reaction mixture were stirred for 3 h at 70° C. in a heating block. Reaction progress was monitored using LC-MS. Once completed, acetonitrile was removed under vacuum, and crude was redissolved in DCM and washed with water (×2) and brine (×1). The organic layer was dried over sodium sulfate and concentrated under vacuum to afford methyl 2-bromo-4-ethoxybenzoate. ESI MS m/z=260.9 [M+H]⁺. Crude was transferred to the next reaction without any further purification.

### Step 2

In a 40 mL vial equipped with a stir bar, methyl 2-bromo-4-ethoxybenzoate (561 mg, 1.0 equiv.), cyclopropylboronic acid (465 mg, 2.5 equiv., CAS #411235-57-9), [1,1′-Bis(diphenylphosphino)ferrocene]dichloropalladium(II) (158 mg, 0.1 equiv. CAS #: 72287-26-4) and potassium phosphate tribasic (1.15 g, 2.5 equiv., CAS #: 7778-53-2) were combined neat under nitrogen atmosphere and followed by addition of 1,4-dioxane (8.66 mL) and water (2.17 mL). The reaction mixture was heated at 100° C. for 2 h in a heating block. After completion, the crude reaction mixture was concentrated and redissolved in THF (10 mL) and followed by added LiOH (10.8 mL, 5.0 equiv., 1.0 M solⁿ in H₂O). The reaction mixture was heated at 60° C. for 12 h in a heating block. Upon cooling to the room temperature, the reaction mixture was quenched with 1N HCl and extracted with ethyl acetate (×3). The combined organic layer was further washed with water (×1) and brine (×1). The organic layer was dried over sodium sulfate and concentrated under vacuum. The crude residue was purified through normal phase silica gel column chromatography (cyclohexane/ethyl acetate) to afford 2-cyclopropyl-4-ethoxybenzoic acid (352 mg, 78.8% yield). ESI MS m/z=207.0 [M+H]⁺.

### Step 3

In a 8 mL vial equipped with a stir bar, 2-bromo-4-ethoxybenzoic acid (100 mg, 1.0 equiv.), oxazol-5-ylmethanamine hydrochloride (60.4 mg, 1.1 equiv., CAS #1196156-45-2), and HATU (248 mg, 1.6 equiv., CAS #148893-10-1) were combined neat under nitrogen atmosphere and followed by addition of dry DCM (2.0 mL) at room temperature. DIPEA (0.23 mL, 3.2 equiv.) was added dropwise, and reaction mixture was stirred for 1 h at 25° C. Reaction progress was monitored using LC-MS. Once completed, DCM was removed under vacuum, and crude was redissolved in 3.0 mL of dimethyl sulfoxide, and purified through reversed phase column chromatography (acetonitrile/water) to afford 2-cyclopropyl-4-ethoxy-N-(oxazol-5-ylmethyl)benzamide (110 mg, 94% yield). ESI MS m/z=287.4 [M+H]⁺.

### Step 4

In a 4 mL vial equipped with a stir bar, 2-cyclopropyl-4-ethoxy-N-(oxazol-5-ylmethyl)benzamide (37 mg, 1.1 equiv.) and ethyl 2-amino-7-((tert-butyldimethylsilyl)oxy)-6-chlorobenzo[b]thiophene-3-carboxylate (50 mg, 1.0 equiv.) were combined neat under nitrogen atmosphere and followed by addition of dry DCE (0.58 mL). Phosphorus(V) oxychloride (27 μL, 2.5 equiv., CAS #: 10025-87-3) was added dropwise and reaction mixture was stirred for 6 h at 80° C. in a heating block. Reaction progress was monitored using LC-MS. Upon cooling to room temperature, solvent (DCE) was removed, and crude was redissolved in MeOH (0.58 mL) and followed by addition of sodium methoxide (25 wt % in MeOH, 0.13 mL, 5.0 equiv.). The reaction mixture was heated at 65° C. for 2 h in a heating block. Upon cooling to room temperature, the reaction mixture was quenched with 1N HCl, concentrated under vacuum. The crude was redissolved in 3.0 mL of dimethyl sulfoxide, and purified through RPHPLC to afford 7-chloro-2-(2-cyclopropyl-4-ethoxyphenyl)-8-hydroxy-3-(oxazol-5-ylmethyl)benzo[4,5]thieno[2,3-d]pyrimidin-4(3H)-one (3.8 mg, 6.6% yield). ESI MS m/z=494.0 [M+H]⁺.

The following compounds were prepared using a procedure analogous to that described for example 72

| Example # | Structure |
|---|---|
| 73 | 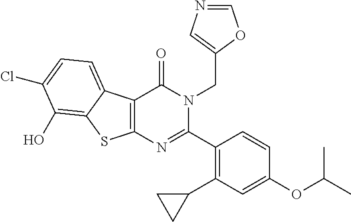 [SMILES: CC(C)Oc1ccc(-c2nc3sc4c(O)c(Cl)ccc4c3c(=O)n2Cc2cnco2)c(C2CC2)c1] |
| 74 | 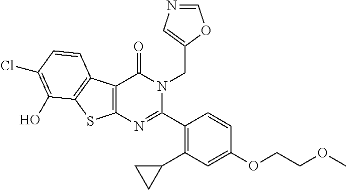 [SMILES: COCCOc1ccc(-c2nc3sc4c(O)c(Cl)ccc4c3c(=O)n2Cc2cnco2)c(C2CC2)c1] |

### Synthesis of 2-(4-(tert-butyl)-2-cyclopropylphenyl)-7-chloro-8-hydroxy-3-(oxazol-5-ylmethyl)benzo[4,5]thieno[2,3-d]pyrimidin-4(3H)-one (Example 75)

### Example 75

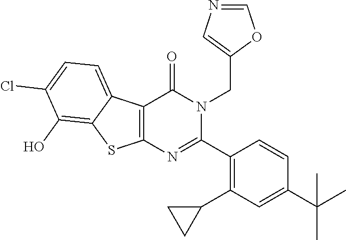 [SMILES: CC(C)(C)c1ccc(-c2nc3sc4c(O)c(Cl)ccc4c3c(=O)n2Cc2cnco2)c(C2CC2)c1]

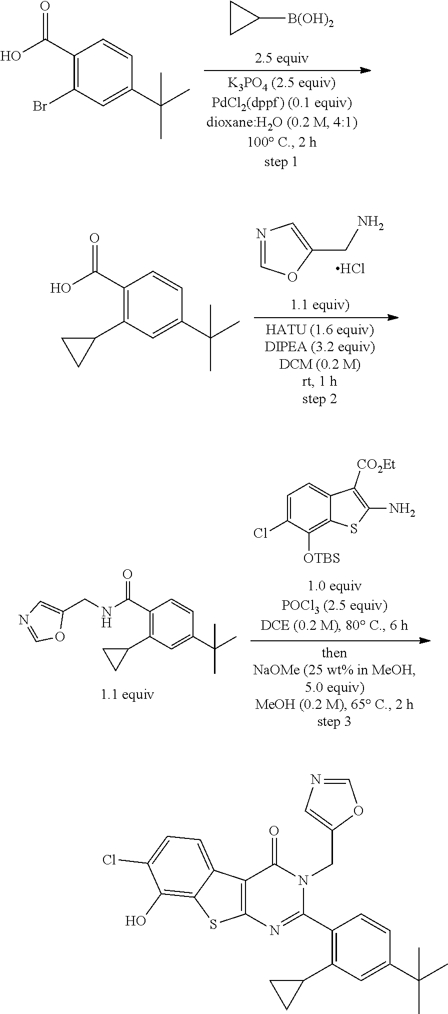

### Step 1

In a 20 mL vial equipped with a stir bar, 2-bromo-4-(tert-butyl)benzoic acid (300 mg, 1.0 equiv.), cyclopropylboronic acid (251 mg, 2.5 equiv., CAS #411235-57-9), [1,1′-Bis(diphenylphosphino)ferrocene]dichloropalladium(II) (85.4 mg, 0.1 equiv. CAS #: 72287-26-4) and potassium phosphate tribasic (619 mg, 2.5 equiv., CAS #: 7778-53-2) were combined neat under nitrogen atmosphere and followed by addition of 1,4-dioxane (4.67 mL) and water (1.17 mL). The reaction mixture was heated at 100° C. for 2 h in a heating block. After completion, solvents were removed under vacuum and crude was redissolved in 3.0 mL of dimethyl sulfoxide, and purified through reversed phase column chromatography (acetonitrile/water) to afford 4-(tert-butyl)-2-cyclopropylbenzoic acid (52.8 mg, 21% yield). ESI MS m/z=219.1 [M+H]⁺.

### Step 2

In an 8 mL vial equipped with a stir bar, 4-(tert-butyl)-2-cyclopropylbenzoic acid (66 mg, 1.0 equiv.), oxazol-5-ylmethanamine hydrochloride (61 mg, 1.5 equiv., CAS #1196156-45-2), and HATU (230 mg, 2.0 equiv., CAS #148893-10-1) were combined neat under nitrogen atmosphere and followed by addition of dry DCM (1.5 mL) at room temperature. DIPEA (0.21 mL, 4.0 equiv.) was added dropwise, and reaction mixture was stirred for 1 h at 25° C. Reaction progress was monitored using LC-MS. Once completed, DCM was removed under vacuum, and crude was redissolved in 3.0 mL of dimethyl sulfoxide, and purified through reversed phase column chromatography (acetonitrile/water) to afford 4-(tert-butyl)-2-cyclopropyl-N-(oxazol-5-ylmethyl)benzamide (65 mg, 72% yield). ESI MS m/z=299.4 [M+H]⁺.

### Step 3

In a 4 mL vial equipped with a stir bar, 4-(tert-butyl)-2-cyclopropyl-N-(oxazol-5-ylmethyl)benzamide (31 mg, 1.1 equiv.) and ethyl 2-amino-7-((tert-butyldimethylsilyl)oxy)-6-chlorobenzo[b]thiophene-3-carboxylate (40 mg, 1.0 equiv.) were combined neat under nitrogen atmosphere and followed by addition of dry DCE (0.47 mL). Phosphorus(V) oxychloride (23 μL, 2.5 equiv., CAS #: 10025-87-3) was added dropwise and reaction mixture was stirred for 6 h at 80° C. in a heating block. Reaction progress was monitored using LC-MS. Upon cooling to room temperature, solvent (DCE) was removed, and crude was redissolved in MeOH (0.47 mL) and followed by addition of sodium methoxide (25 wt % in MeOH, 0.11 mL, 5.0 equiv.). The reaction mixture was heated at 65° C. for 2 h in a heating block. Upon cooling to room temperature, the reaction mixture was quenched with 1N HCl, concentrated under vacuum. The crude was redissolved in 2.0 mL of dimethyl sulfoxide, and purified through RPHPLC to afford 2-(4-(tert-butyl)-2-cyclopropylphenyl)-7-chloro-8-hydroxy-3-(oxazol-5-ylmethyl)benzo[4,5]thieno[2,3-d]pyrimidin-4(3H)-one (21.0 mg, 44% yield). ESI MS m/z=506.1 [M+H]⁺.

### Synthesis of 7-chloro-2-(2-cyclopropyl-5-fluoro-4-methoxyphenyl)-8-hydroxy-3-(oxazol-5-ylmethyl)benzo[4,5]thieno[2,3-d]pyrimidin-4(3H)-one (Example 76)

### Example 76

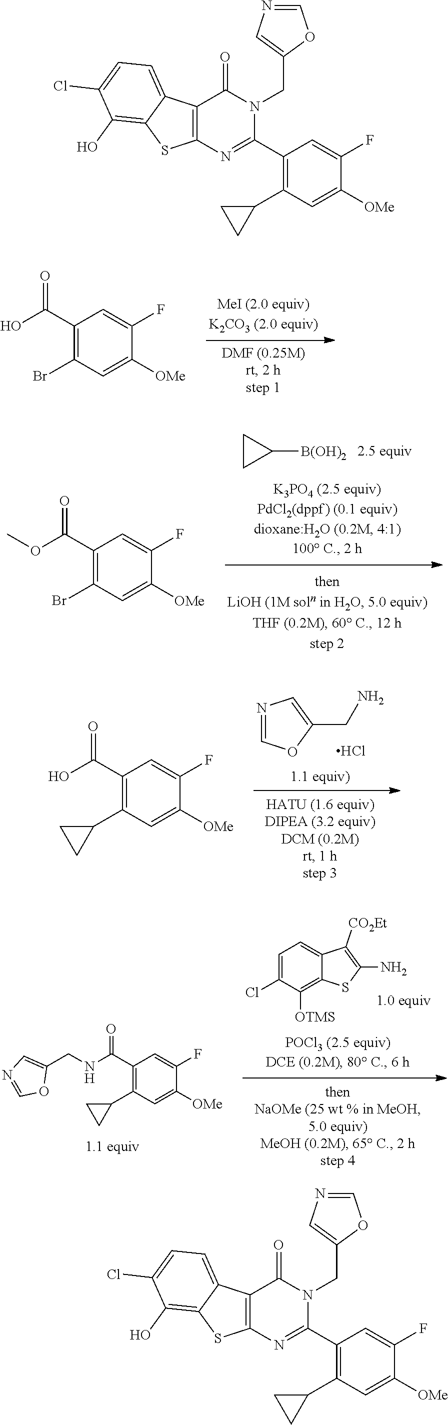

### Step 1

In a 20 mL vial equipped with a stir bar, 2-bromo-5-fluoro-4-methoxybenzoic acid (250 mg, 1.0 equiv., CAS #64695-99-4) was dissolved in dry DMF (4.0 mL) at room temperature, followed by added potassium carbonate (285 mg, 2.0 equiv.). Subsequently, iodomethane (0.13 mL, 2.0 equiv.) was added dropwise and reaction mixture were stirred for 2 h at room temperature. Reaction progress was monitored using LC-MS. Once completed, the reaction mixture was diluted with ethyl acetate and washed with water. The aqueous layer was further extracted with ethyl acetate three additional times. Then combined organic layers were washed with water (×2) and brine (×1). The organic layer was dried over sodium sulfate and concentrated under vacuum to afford methyl 2-bromo-5-fluoro-4-methoxybenzoate. ESI MS m/z=262.9 [M+H]⁺. Crude was transferred to the next reaction without any further purification.

### Step 2

In a 20 mL vial equipped with a stir bar, methyl 2-bromo-5-fluoro-4-methoxybenzoate (260 mg, 1.0 equiv.), cyclopropylboronic acid (212 mg, 2.5 equiv., CAS #411235-57-9), [1,1′-Bis(diphenylphosphino)ferrocene]dichloropalladium(II) (72.3 mg, 0.1 equiv. CAS #: 72287-26-4) and potassium phosphate tribasic (525 mg, 2.5 equiv., CAS #: 7778-53-2) were combined neat under nitrogen atmosphere and followed by addition of 1,4-dioxane (4.0 mL) and water (1.0 mL). The reaction mixture was heated at 100° C. for 2 h in a heating block. After completion, the crude reaction mixture was concentrated and redissolved in THF (5 mL) and followed by added LiOH (5.0 mL, 5.0 equiv., 1.0 M solⁿ in H₂O). The reaction mixture was heated at 60° C. for 12 h in a heating block. Upon cooling to the room temperature, the reaction mixture was quenched with 1N HCl and extracted with ethyl acetate (×3). The combined organic layer was further washed with water (×1) and brine (×1). The organic layer was dried over sodium sulfate and concentrated under vacuum. The crude residue was purified through normal phase silica gel column chromatography (cyclohexane/ethyl acetate) to afford 2-cyclopropyl-5-fluoro-4-methoxybenzoic acid (178 mg, 85.7% yield). ESI MS m/z=211.0 [M+H]⁺.

### Step 3

In a 8 mL vial equipped with a stir bar, 2-cyclopropyl-5-fluoro-4-methoxybenzoic acid (60 mg, 1.0 equiv.), oxazol-5-ylmethanamine hydrochloride (42 mg, 1.1 equiv., CAS #1196156-45-2), and HATU (170 mg, 1.6 equiv., CAS #148893-10-1) were combined neat under nitrogen atmosphere and followed by addition of dry DCM (1.4 mL) at room temperature. DIPEA (0.16 mL, 3.2 equiv.) was added dropwise, and reaction mixture was stirred for 1 h at room temperature. Reaction progress was monitored using LC-MS. Once completed, DCM was removed under vacuum, and crude was redissolved in 3.0 mL of dimethyl sulfoxide, and purified through reversed phase column chromatography (acetonitrile/water) to afford 2-cyclopropyl-5-fluoro-4-methoxy-N-(oxazol-5-ylmethyl)benzamide (71 mg, 86% yield). ESI MS m/z=291.2 [M+H]⁺.

### Step 4

In a 4 mL vial equipped with a stir bar, 2-cyclopropyl-5-fluoro-4-methoxy-N-(oxazol-5-ylmethyl)benzamide (37 mg, 1.1 equiv.) and ethyl 2-amino-7-((tert-butyldimethylsilyl)oxy)-6-chlorobenzo[b]thiophene-3-carboxylate (50 mg, 1.0 equiv.) were combined neat under nitrogen atmosphere and followed by addition of dry DCE (0.58 mL). Phosphorus(V) oxychloride (27 μL, 2.5 equiv., CAS #: 10025-87-3) was added dropwise and reaction mixture was stirred for 6 h at 80° C. in a heating block. Reaction progress was monitored using LC-MS. Upon cooling to room temperature, solvent (DCE) was removed, and crude was redissolved in MeOH (0.58 mL) and followed by addition of sodium methoxide (25 wt % in MeOH, 0.13 mL, 5.0 equiv.). The reaction mixture was heated at 65° C. for 2 h in a heating block. Upon cooling to room temperature, the reaction mixture was quenched with 1N HCl, concentrated under vacuum. The crude was redissolved in 3.0 mL of dimethyl sulfoxide and purified through RPHPLC to afford 7-chloro-2-(2-cyclopropyl-5-fluoro-4-methoxyphenyl)-8-hydroxy-3-(oxazol-5-ylmethyl)benzo[4,5]thieno[2,3-d]pyrimidin-4(3H)-one (1.37 mg, 2.4% yield). ESI MS m/z=498.0 [M+H]⁺.

The following compounds were prepared using a procedure analogous to that described for example 76

| Example # | Structure |
|---|---|
| 77 | 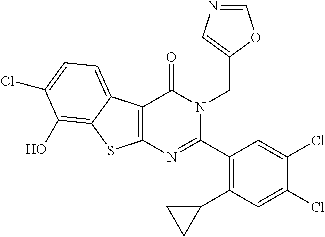 [SMILES: O=c1c2c(nc(-c3cc(Cl)c(Cl)cc3C3CC3)n1Cc1cnco1)sc1c(O)c(Cl)ccc12] |
| 78 | 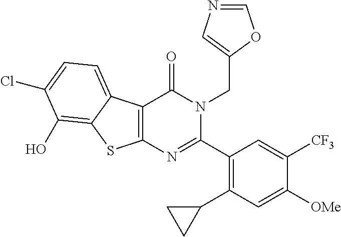 [SMILES: COc1cc(C2CC2)c(-c2nc3sc4c(O)c(Cl)ccc4c3c(=O)n2Cc2cnco2)cc1C(F)(F)F] |
| 79 | 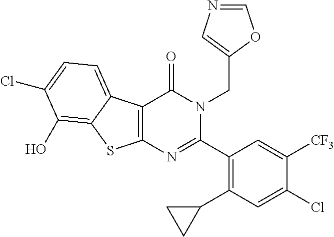 [SMILES: O=c1c2c(nc(-c3cc(C(F)(F)F)c(Cl)cc3C3CC3)n1Cc1cnco1)sc1c(O)c(Cl)ccc12] |
| 80 | 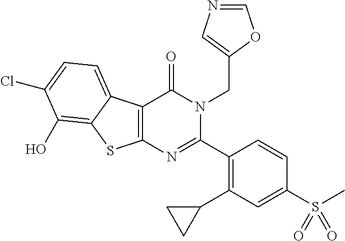 [SMILES: CS(=O)(=O)c1ccc(-c2nc3sc4c(O)c(Cl)ccc4c3c(=O)n2Cc2cnco2)c(C2CC2)c1] |
| 81 | 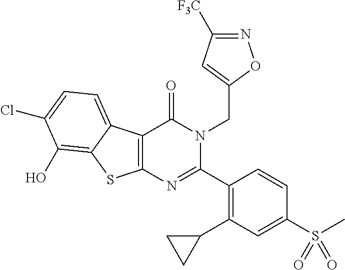 [SMILES: CS(=O)(=O)c1ccc(-c2nc3sc4c(O)c(Cl)ccc4c3c(=O)n2Cc2cc(C(F)(F)F)no2)c(C2CC2)c1] |

### Synthesis of 7-chloro-2-(2-cyclopropyl-4-fluoro-5-methoxyphenyl)-8-hydroxy-3-(oxazol-5-ylmethyl)benzo[4,5]thieno[2,3-d]pyrimidin-4 (3H)-one (Example 82)

### Example 82

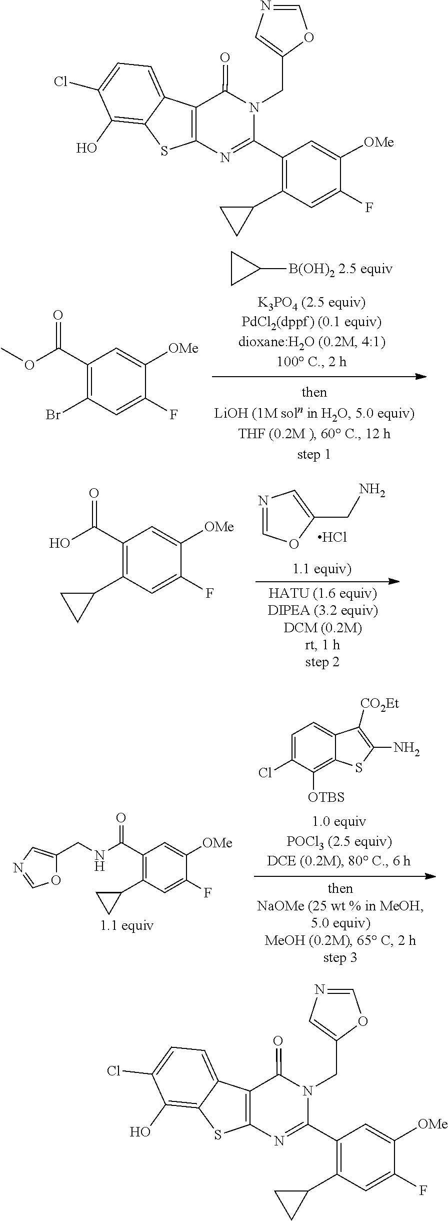

### Step 1

In a 40 mL vial equipped with a stir bar, methyl 2-bromo-4-fluoro-5-methoxybenzoate (500 mg, 1.0 equiv., CAS #1007455-22-2), cyclopropylboronic acid (408 mg, 2.5 equiv., CAS #411235-57-9), [1,1′-Bis(diphenylphosphino)ferrocene]dichloropalladium(II) (139 mg, 0.1 equiv. CAS #: 72287-26-4) and potassium phosphate tribasic (1.01 g, 2.5 equiv., CAS #: 7778-53-2) were combined neat under nitrogen atmosphere and followed by addition of 1,4-dioxane (7.6 mL) and water (1.9 mL). The reaction mixture was heated at 100° C. for 2 h in a heating block. After completion, the crude reaction mixture was concentrated and redissolved in THF (10 mL) and followed by added LiOH (9.5 mL, 5.0 equiv., 1.0 M solⁿ in H₂O). The reaction mixture was heated at 60° C. for 12 h in a heating block. Upon cooling to room temperature, the reaction mixture was quenched with 1N HCl and extracted with ethyl acetate (×3). The combined organic layer was further washed with water (×1) and brine (×1). The organic layer was dried over sodium sulfate and concentrated under vacuum. The crude was redissolved in 3.0 mL of dimethyl sulfoxide and purified through reversed phase column chromatography (acetonitrile/water) to afford 2-cyclopropyl-4-fluoro-5-methoxybenzoic acid. ESI MS m/z=211.0 [M+H]⁺.

### Step 2

In a 8 mL vial equipped with a stir bar, 2-cyclopropyl-4-fluoro-5-methoxybenzoic acid (100 mg, 1.0 equiv.), oxazol-5-ylmethanamine hydrochloride (70.4 mg, 1.1 equiv., CAS #1196156-45-2), and HATU (289 mg, 1.6 equiv., CAS #148893-10-1) were combined neat under nitrogen atmosphere and followed by addition of dry DCM (1.4 mL) at room temperature. DIPEA (0.26 mL, 3.2 equiv.) was added dropwise, and reaction mixture was stirred for 1 h at room temperature. Reaction progress was monitored using LC-MS. Once completed, DCM was removed under vacuum, and crude was redissolved in 3.0 mL of dimethyl sulfoxide, and purified through reversed phase column chromatography (acetonitrile/water) to afford 2-cyclopropyl-4-fluoro-5-methoxy-N-(oxazol-5-ylmethyl)benzamide. ESI MS m/z=291.2 [M+H]⁺.

### Step 3

In a 4 mL vial equipped with a stir bar, 2-cyclopropyl-4-fluoro-5-methoxy-N-(oxazol-5-ylmethyl)benzamide (37 mg, 1.1 equiv.) and ethyl 2-amino-7-((tert-butyldimethylsilyl)oxy)-6-chlorobenzo[b]thiophene-3-carboxylate (50 mg, 1.0 equiv.) were combined neat under nitrogen atmosphere and followed by addition of dry DCE (0.58 mL). Phosphorus(V) oxychloride (27 μL, 2.5 equiv., CAS #: 10025-87-3) was added dropwise and reaction mixture was stirred for 6 h at 80° C. in a heating block. Reaction progress was monitored using LC-MS. Upon cooling to room temperature, solvent (DCE) was removed, and crude was redissolved in MeOH (0.58 mL) and followed by addition of sodium methoxide (25 wt % in MeOH, 0.13 mL, 5.0 equiv.). The reaction mixture was heated at 65° C. for 2 h in a heating block. Upon cooling to room temperature, the reaction mixture was quenched with 1N HCl, concentrated under vacuum. The crude was redissolved in 3.0 mL of dimethyl sulfoxide and purified through RPHPLC to afford 7-chloro-2-(2-cyclopropyl-4-fluoro-5-methoxyphenyl)-8-hydroxy-3-(oxazol-5-ylmethyl)benzo[4,5]thieno[2,3-d]pyrimidin-4(3H)-one (25 mg, 43% yield). ESI MS m/z=498.0 [M+H]⁺.

The following compounds were prepared using a procedure analogous to that described for example 82

| Example # | Structure |
|---|---|
| 83 | 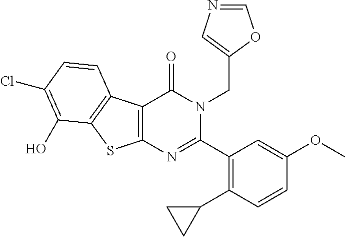 [SMILES: COc1ccc(C2CC2)c(-c2nc3sc4c(O)c(Cl)ccc4c3c(=O)n2Cc2cnco2)c1] |
| 84 | 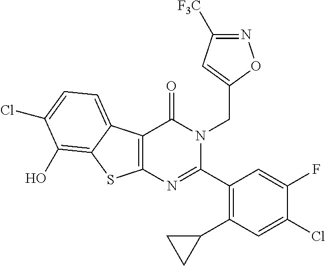 [SMILES: O=c1c2c(nc(-c3cc(F)c(Cl)cc3C3CC3)n1Cc1cc(C(F)(F)F)no1)sc1c(O)c(Cl)ccc12] |
| 85 | 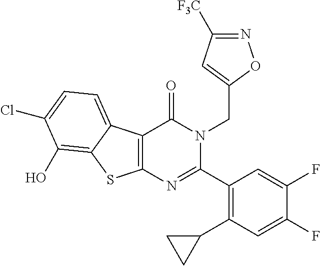 [SMILES: O=c1c2c(nc(-c3cc(F)c(F)cc3C3CC3)n1Cc1cc(C(F)(F)F)no1)sc1c(O)c(Cl)ccc12] |
| 86 | 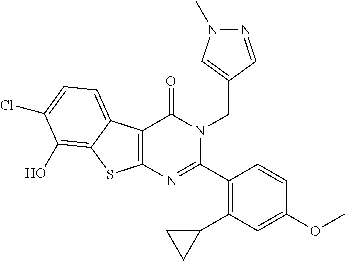 [SMILES: COc1ccc(-c2nc3sc4c(O)c(Cl)ccc4c3c(=O)n2Cc2cnn(C)c2)c(C2CC2)c1] |
| 87 | 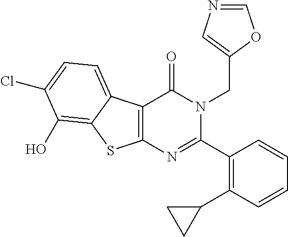 [SMILES: O=c1c2c(nc(-c3ccccc3C3CC3)n1Cc1cnco1)sc1c(O)c(Cl)ccc12] |
| 88 | 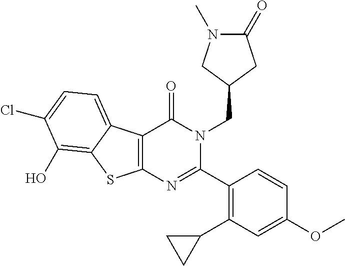 [SMILES: COc1ccc(-c2nc3sc4c(O)c(Cl)ccc4c3c(=O)n2C[C@@H]2CC(=O)N(C)C2)c(C2CC2)c1] |
| 89 | 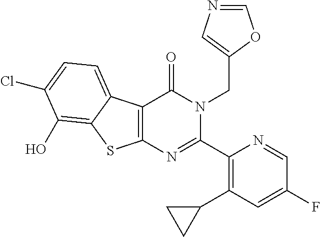 [SMILES: O=c1c2c(nc(-c3ncc(F)cc3C3CC3)n1Cc1cnco1)sc1c(O)c(Cl)ccc12] |
| 90 | 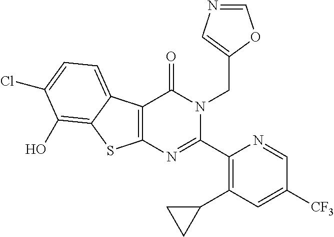 [SMILES: O=c1c2c(nc(-c3ncc(C(F)(F)F)cc3C3CC3)n1Cc1cnco1)sc1c(O)c(Cl)ccc12] |
| 91 | 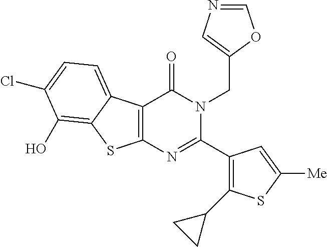 [SMILES: Cc1cc(-c2nc3sc4c(O)c(Cl)ccc4c3c(=O)n2Cc2cnco2)c(C2CC2)s1] |
| 92 | 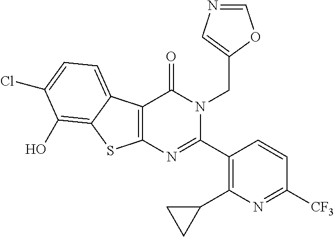 [SMILES: O=c1c2c(nc(-c3ccc(C(F)(F)F)nc3C3CC3)n1Cc1cnco1)sc1c(O)c(Cl)ccc12] |
| 93 | 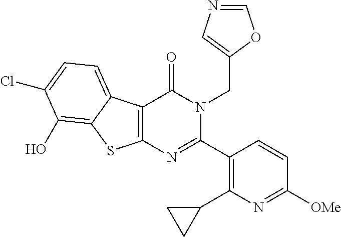 [SMILES: COc1ccc(-c2nc3sc4c(O)c(Cl)ccc4c3c(=O)n2Cc2cnco2)c(C2CC2)n1] |
| 94 | 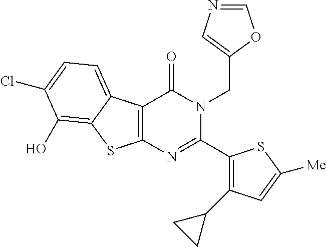 [SMILES: Cc1cc(C2CC2)c(-c2nc3sc4c(O)c(Cl)ccc4c3c(=O)n2Cc2cnco2)s1] |
| 95 | 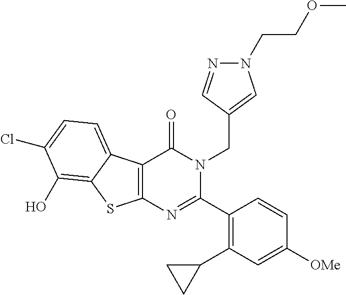 [SMILES: COCCn1cc(Cn2c(-c3ccc(OC)cc3C3CC3)nc3sc4c(O)c(Cl)ccc4c3c2=O)cn1] |
| 96 | 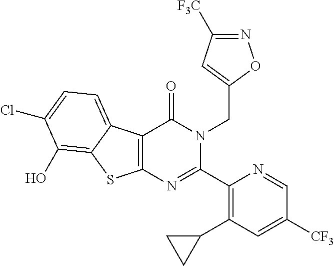 [SMILES: O=c1c2c(nc(-c3ncc(C(F)(F)F)cc3C3CC3)n1Cc1cc(C(F)(F)F)no1)sc1c(O)c(Cl)ccc12] |
| 97 | 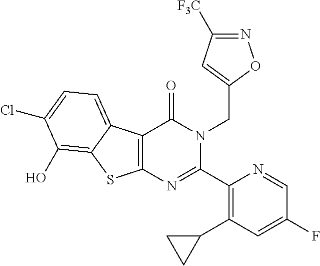 [SMILES: O=c1c2c(nc(-c3ncc(F)cc3C3CC3)n1Cc1cc(C(F)(F)F)no1)sc1c(O)c(Cl)ccc12] |
| 98 | 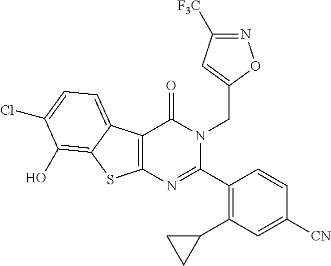 [SMILES: N#Cc1ccc(-c2nc3sc4c(O)c(Cl)ccc4c3c(=O)n2Cc2cc(C(F)(F)F)no2)c(C2CC2)c1] |
| 99 | 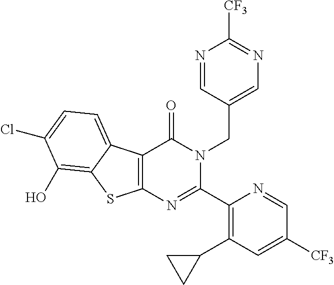 [SMILES: O=c1c2c(nc(-c3ncc(C(F)(F)F)cc3C3CC3)n1Cc1cnc(C(F)(F)F)nc1)sc1c(O)c(Cl)ccc12] |
| 100 | 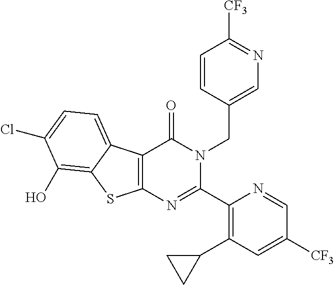 [SMILES: O=c1c2c(nc(-c3ncc(C(F)(F)F)cc3C3CC3)n1Cc1ccc(C(F)(F)F)nc1)sc1c(O)c(Cl)ccc12] |
| 101 | 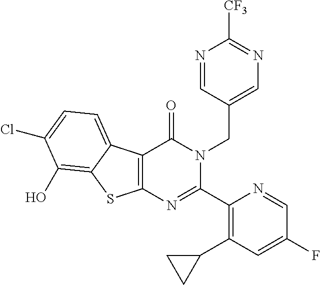 [SMILES: O=c1c2c(nc(-c3ncc(F)cc3C3CC3)n1Cc1cnc(C(F)(F)F)nc1)sc1c(O)c(Cl)ccc12] |
| 102 | 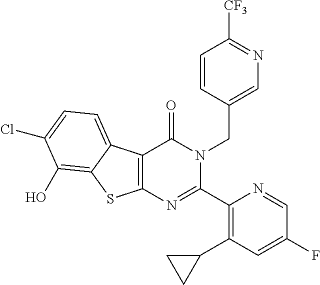 [SMILES: O=c1c2c(nc(-c3ncc(F)cc3C3CC3)n1Cc1ccc(C(F)(F)F)nc1)sc1c(O)c(Cl)ccc12] |
| 103 | 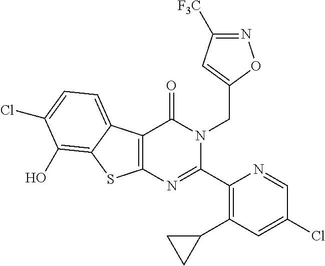 [SMILES: O=c1c2c(nc(-c3ncc(Cl)cc3C3CC3)n1Cc1cc(C(F)(F)F)no1)sc1c(O)c(Cl)ccc12] |

### Synthesis of 2-(5-bromo-2-cyclopropyl-4-methoxyphenyl)-7-chloro-8-hydroxy-3-(oxazol-5-ylmethyl)benzo[4,5]thieno[2,3-d]pyrimidin-4(3H)-one (Example 104)

### Example 104

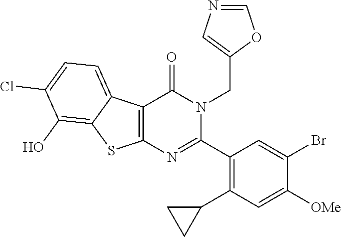 [SMILES: COc1cc(C2CC2)c(-c2nc3sc4c(O)c(Cl)ccc4c3c(=O)n2Cc2cnco2)cc1Br]

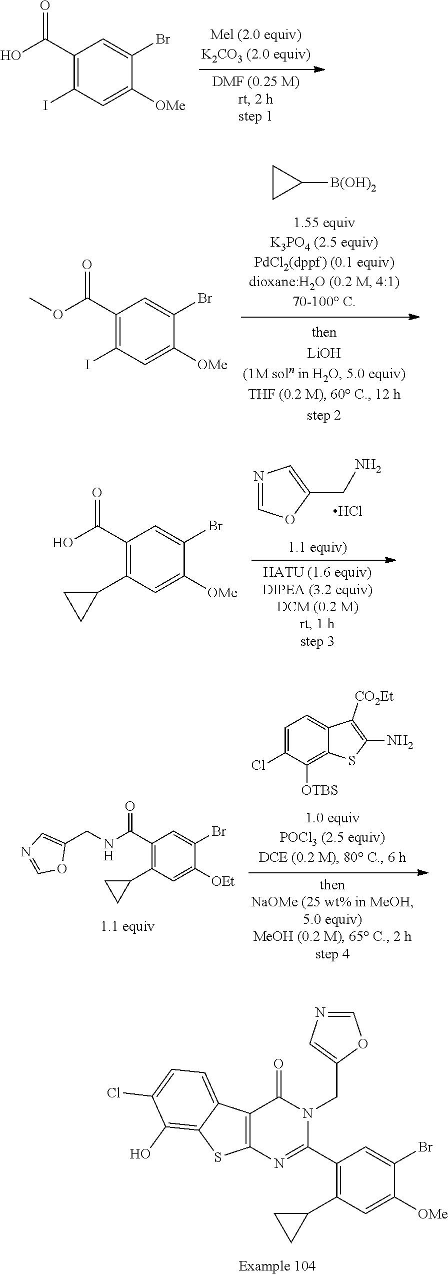

### Step 1

In a 40 mL vial equipped with a stir bar, 5-bromo-2-iodo-4-methoxybenzoic acid (1.0 g, 1.0 equiv., CAS #1269292-64-9) was dissolved in dry DMF (9.4 mL) at room temperature, followed by added potassium carbonate (774 mg, 2.0 equiv.). Subsequently, iodomethane (0.35 mL, 2.0 equiv.) was added dropwise and reaction mixture were stirred for 2 h at room temperature. Reaction progress was monitored using LC-MS. Once completed, the reaction mixture was diluted with ethyl acetate and washed with water. The aqueous layer was further extracted with ethyl acetate three additional times. Then combined organic layers were washed with water (×2) and brine (×1). The organic layer was dried over sodium sulfate and concentrated under vacuum to afford methyl 5-bromo-2-iodo-4-methoxybenzoate. ESI MS m/z=370.8 [M+H]⁺. Crude was transferred to the next reaction without any further purification.

### Step 2

In a 100 mL round bottom flask equipped with a stir bar, methyl 5-bromo-2-iodo-4-methoxybenzoate (1.0 g, 1.0 equiv.), cyclopropylboronic acid (243 mg, 1.05 equiv., CAS #411235-57-9), [1,1′-Bis(diphenylphosphino)ferrocene]dichloropalladium(II) (197 mg, 0.1 equiv. CAS #: 72287-26-4) and potassium phosphate tribasic (1.43 g, 2.5 equiv., CAS #: 7778-53-2) were combined neat under nitrogen atmosphere and followed by addition of 1,4-dioxane (10.8 mL) and water (2.7 mL). The reaction mixture was heated at 70° C. for 16 h in a heating block. Reaction progress was monitored using LC-MS. As the starting material was not consumed completely, 0.5 equiv. cyclopropylboronic acid (116 mg) and 0.05 equiv. Pd catalyst (99 mg) were added to the reaction mixture and the mixture was heated for 2 h at 100° C. in a heating block. After completion, the crude reaction mixture was concentrated under vacuum and redissolved in THF (27 mL) and followed by addition of LiOH (27 mL, 5.0 equiv., 1.0 M solⁿ in H₂O). The reaction mixture was heated at 60° C. for 12 h in a heating block. Upon cooling to the room temperature, the reaction mixture was quenched with 1N HCl and extracted with ethyl acetate (×3). The combined organic layer was further washed with water (×1) and brine (×1). The organic layer was dried over sodium sulfate and concentrated under vacuum then crude was redissolved in dimethyl sulfoxide and purified through reversed phase column chromatography (acetonitrile/water) to afford 5-bromo-2-cyclopropyl-4-methoxybenzoic acid (430 mg, 59% yield). ESI MS m/z=272.8 [M+H]⁺.

### Step 3

In a 40 mL vial equipped with a stir bar, 5-bromo-2-cyclopropyl-4-methoxybenzoic acid (430 mg, 1.0 equiv.), oxazol-5-ylmethanamine hydrochloride (256 mg, 1.1 equiv., CAS #1196156-45-2), and HATU (965 mg, 1.6 equiv., CAS #148893-10-1) were combined neat under nitrogen atmosphere and followed by addition of dry DCM (8.0 mL) at room temperature. DIPEA (0.9 mL, 3.2 equiv.) was added dropwise, and reaction mixture was stirred for 1 h at room temperature. Reaction progress was monitored using LC-MS. Once completed, DCM was removed under vacuum, and crude was redissolved in dimethyl sulfoxide, and purified through reversed phase column chromatography (acetonitrile/water) to afford 5-bromo-2-cyclopropyl-4-methoxy-N-(oxazol-5-ylmethyl)benzamide. ESI MS m/z=352.9 [M+H]⁺.

### Step 4

In a 4 mL vial equipped with a stir bar, 5-bromo-2-cyclopropyl-4-methoxy-N-(oxazol-5-ylmethyl)benzamide (27 mg, 1.1 equiv.) and ethyl 2-amino-7-((tert-butyldimethylsilyl)oxy)-6-chlorobenzo[b]thiophene-3-carboxylate (30 mg, 1.0 equiv.) were combined neat under nitrogen atmosphere and followed by addition of dry DCE (0.35 mL). Phosphorus(V) oxychloride (16 μL, 2.5 equiv., CAS #: 10025-87-3) was added dropwise and reaction mixture was stirred for 6 h at 80° C. in a heating block. Reaction progress was monitored using LC-MS. Upon cooling to room temperature, solvent (DCE) was removed, and crude was redissolved in MeOH (0.35 mL) and followed by addition of sodium methoxide (25 wt % in MeOH, 0.08 mL, 5.0 equiv.). The reaction mixture was heated at 65° C. for 2 h in a heating block. Upon cooling to room temperature, the reaction mixture was quenched with 1N HCl, concentrated under vacuum. The crude was redissolved in 3.0 mL of dimethyl sulfoxide and purified through RPHPLC to afford 2-(5-bromo-2-cyclopropyl-4-methoxyphenyl)-7-chloro-8-hydroxy-3-(oxazol-5-ylmethyl)benzo[4,5]thieno[2,3-d]pyrimidin-4(3H)-one (24 mg, 58% yield). ESI MS m/z=559.8 [M+H]⁺.

### Synthesis of 7-chloro-2-(5-chloro-2-cyclopropyl-4-methoxyphenyl)-8-hydroxy-3-(oxazol-5-ylmethyl)benzo[4,5]thieno[2,3-d]pyrimidin-4(3H)-one (Example 105)

### Example 105

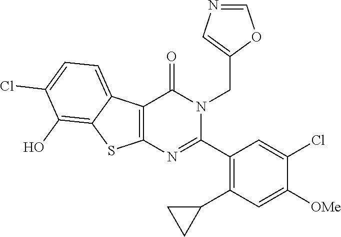 [SMILES: COc1cc(C2CC2)c(-c2nc3sc4c(O)c(Cl)ccc4c3c(=O)n2Cc2cnco2)cc1Cl]

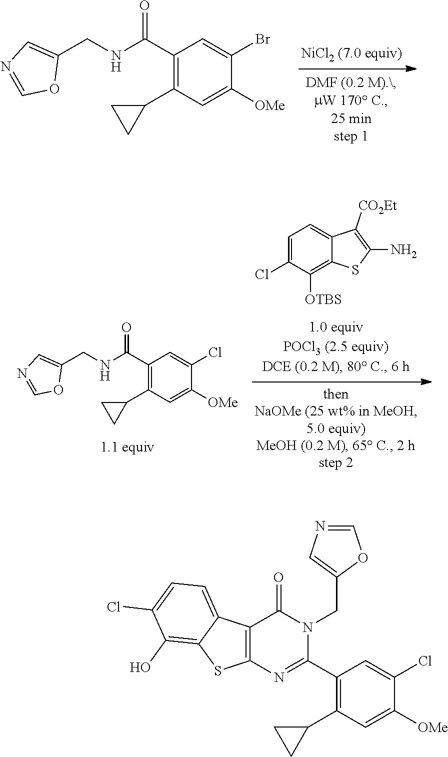 [SMILES: COc1cc(C2CC2)c(C(=O)NCc2cnco2)cc1Br ; CCOC(=O)c1c(N)sc2c(O[Si](C)(C)C(C)(C)C)c(Cl)ccc12 ; COc1cc(C2CC2)c(C(=O)NCc2cnco2)cc1Cl ; COc1cc(C2CC2)c(-c2nc3sc4c(O)c(Cl)ccc4c3c(=O)n2Cc2cnco2)cc1Cl]

### Step 1

In a 2 mL microwave vial equipped with a stir bar, 5-bromo-2-cyclopropyl-4-methoxy-N-(oxazol-5-ylmethyl)benzamide (32 mg, 1.0 equiv.) and nickel chloride (83 mg, 7.0 equiv., CAS #7718-54-9) were combined neat under nitrogen atmosphere and followed by addition of dry DMF (0.46 mL). Reaction mixture was further flushed with nitrogen (×3) and then heated at 170° C. for 25 min under microwave condition. After completion, crude reaction mixture was directly loaded into 30 G gold C₁₈ column and purified through reversed phase column chromatography (acetonitrile/water) to afford 5-chloro-2-cyclopropyl-4-methoxy-N-(oxazol-5-ylmethyl)benzamide (24 mg, 86% yield). ESI MS m/z=307.1 [M+H]⁺.

### Step 2

In a 4 mL vial equipped with a stir bar, 5-chloro-2-cyclopropyl-4-methoxy-N-(oxazol-5-ylmethyl)benzamide (24 mg, 1.1 equiv.) and ethyl 2-amino-7-((tert-butyldimethylsilyl)oxy)-6-chlorobenzo[b]thiophene-3-carboxylate (30 mg, 1.0 equiv.) were combined neat under nitrogen atmosphere and followed by addition of dry DCE (0.35 mL). Phosphorus(V) oxychloride (16 μL, 2.5 equiv., CAS #: 10025-87-3) was added dropwise and reaction mixture was stirred for 6 h at 80° C. in a heating block. Reaction progress was monitored using LC-MS. Upon cooling to room temperature, solvent (DCE) was removed, and crude was redissolved in MeOH (0.35 mL) and followed by addition of sodium methoxide (25 wt % in MeOH, 0.08 mL, 5.0 equiv.). The reaction mixture was heated at 65° C. for 2 h in a heating block. Upon cooling to room temperature, the reaction mixture was quenched with 1N HCl, concentrated under vacuum. The crude was redissolved in 3.0 mL of dimethyl sulfoxide, and purified through RPHPLC to afford 7-chloro-2-(5-chloro-2-cyclopropyl-4-methoxyphenyl)-8-hydroxy-3-(oxazol-5-ylmethyl)benzo[4,5]thieno [2,3-d]pyrimidin-4(3H)-one (12 mg, 32% yield). ESI MS m/z=515.9 [M+H]⁺.

The following compound was prepared using a procedure analogous to that described for example 105

| Example # | Structure |
|---|---|
| 106 | 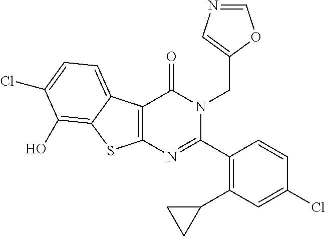 [SMILES: O=c1c2c(nc(-c3ccc(Cl)cc3C3CC3)n1Cc1cnco1)sc1c(O)c(Cl)ccc12] |

### Synthesis of 7-chloro-2-(2,5-dicyclopropyl-4-methoxyphenyl)-8-hydroxy-3-(oxazol-5-ylmethyl)benzo[4,5]thieno[2,3-d]pyrimidin-4(3H)-one (Example 107)

### Example 107

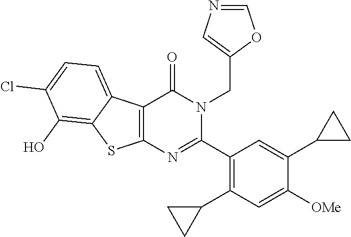 [SMILES: COc1cc(C2CC2)c(-c2nc3sc4c(O)c(Cl)ccc4c3c(=O)n2Cc2cnco2)cc1C1CC1]

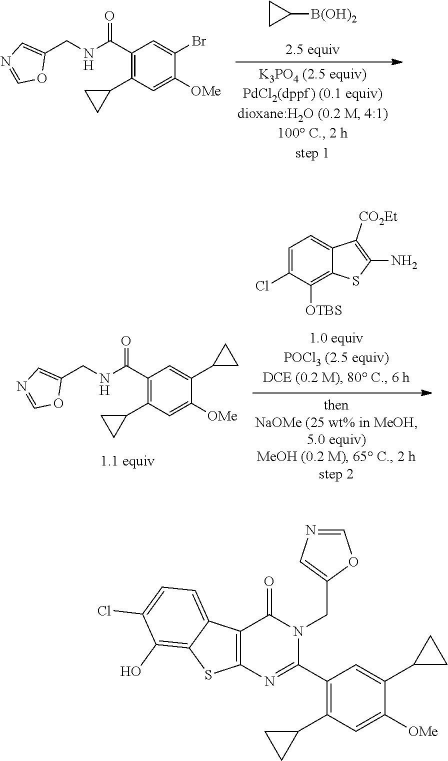 [SMILES: COc1cc(C2CC2)c(C(=O)NCc2cnco2)cc1Br ; OB(O)C1CC1 ; CCOC(=O)c1c(N)sc2c(O[Si](C)(C)C(C)(C)C)c(Cl)ccc12 ; COc1cc(C2CC2)c(C(=O)NCc2cnco2)cc1C1CC1 ; COc1cc(C2CC2)c(-c2nc3sc4c(O)c(Cl)ccc4c3c(=O)n2Cc2cnco2)cc1C1CC1]

### Step 1

In a 4 mL vial equipped with a stir bar, 5-bromo-2-cyclopropyl-4-methoxy-N-(oxazol-5-ylmethyl)benzamide (30 mg, 1.0 equiv.), cyclopropylboronic acid (18 mg, 2.5 equiv., CAS #411235-57-9), [1,1′-Bis(diphenylphosphino)ferrocene]dichloropalladium(II) (6.3 mg, 0.1 equiv. CAS #: 72287-26-4) and potassium phosphate tribasic (45 mg, 2.5 equiv., CAS #: 7778-53-2) were combined neat under nitrogen atmosphere and followed by addition of 1,4-dioxane (0.34 mL) and water (0.085 mL). The reaction mixture was heated at 100° C. for 2 h in a heating block. Upon cooling to room temperature, the reaction mixture was concentrated and was redissolved in 3.0 mL of dimethyl sulfoxide and purified through reversed phase column chromatography (acetonitrile/water) to afford 2,5-dicyclopropyl-4-methoxy-N-(oxazol-5-ylmethyl)benzamide (20 mg, 75% yield). ESI MS m/z=313.3 [M+H]⁺.

### Step 2

In a 4 mL vial equipped with a stir bar, 2,5-dicyclopropyl-4-methoxy-N-(oxazol-5-ylmethyl)benzamide (20 mg, 1.1 equiv.) and ethyl 2-amino-7-((tert-butyldimethylsilyl)oxy)-6-chlorobenzo[b]thiophene-3-carboxylate (26 mg, 1.0 equiv.) were combined neat under nitrogen atmosphere and followed by addition of dry DCE (0.3 mL). Phosphorus(V) oxychloride (14 μL, 2.5 equiv., CAS #: 10025-87-3) was added dropwise and reaction mixture was stirred for 6 h at 80° C. in a heating block. Reaction progress was monitored using LC-MS. Upon cooling to room temperature, solvent (DCE) was removed, and crude was redissolved in MeOH (0.3 mL) and followed by addition of sodium methoxide (25 wt % in MeOH, 0.07 mL, 5.0 equiv.). The reaction mixture was heated at 65° C. for 2 h in a heating block. Upon cooling to room temperature, the reaction mixture was quenched with 1N HCl, concentrated under vacuum. The crude was redissolved in 3.0 mL of dimethyl sulfoxide and purified through RPHPLC to afford 7-chloro-2-(2,5-dicyclopropyl-4-methoxyphenyl)-8-hydroxy-3-(oxazol-5-ylmethyl)benzo[4,5]thieno[2,3-d]pyrimidin-4(3H)-one (11 mg, 35% yield). ESI MS m/z=519.9 [M+H]⁺.

The following compound was prepared using a procedure analogous to that described for example 107

| Example # | Structure |
|---|---|
| 108 | 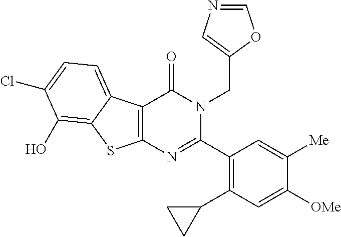 [SMILES: COc1cc(C2CC2)c(-c2nc3sc4c(O)c(Cl)ccc4c3c(=O)n2Cc2cnco2)cc1C] |

### Synthesis of 7-chloro-2-(5-cyclopentyl-2-cyclopropyl-4-methoxyphenyl)-8-hydroxy-3-(oxazol-5-ylmethyl)benzo[4,5]thieno[2,3-d]pyrimidin-4(3H)-one (Example 109)

### Example 109

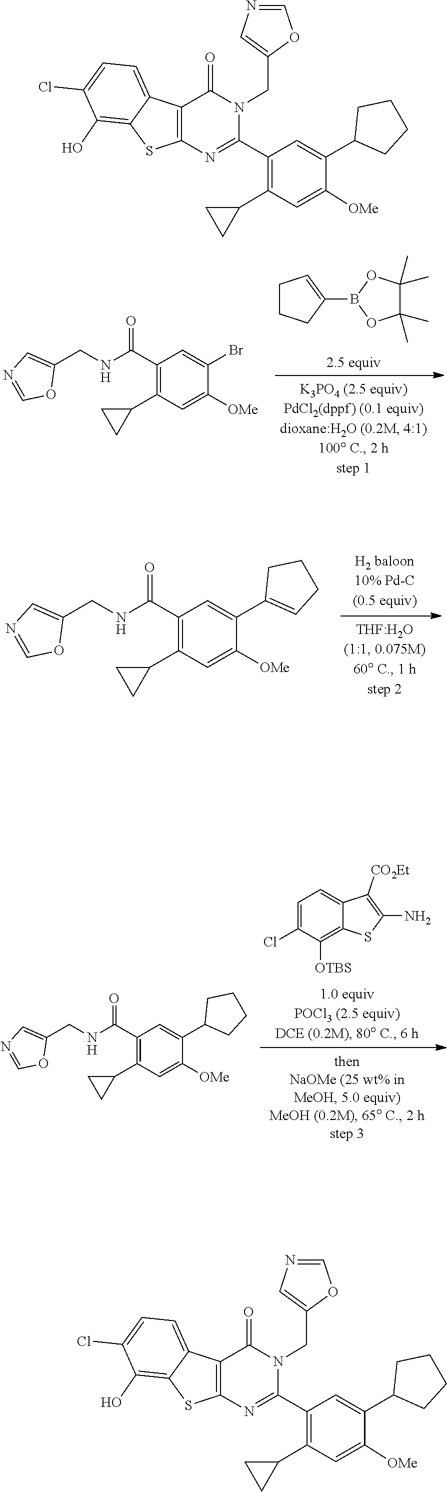

### Step 1

In a 4 mL vial equipped with a stir bar, 5-bromo-2-cyclopropyl-4-methoxy-N-(oxazol-5-ylmethyl)benzamide (50 mg, 1.0 equiv.), 2-(cyclopent-1-en-1-yl)-4,4,5,5-tetramethyl-1,3,2-dioxaborolane (55 mg, 2.5 equiv., CAS #287944-10-9), [1,1′-Bis(diphenylphosphino) ferrocene]dichloropalladium(II) (10 mg, 0.1 equiv. CAS #: 72287-26-4) and potassium phosphate tribasic (76 mg, 2.5 equiv., CAS #: 7778-53-2) were combined neat under nitrogen atmosphere and followed by addition of 1,4-dioxane (0.57 mL) and water (0.14 mL). The reaction mixture was heated at 100° C. for 2 h in a heating block. Upon cooling to room temperature, the reaction mixture was concentrated and was redissolved in 3.0 mL of dimethyl sulfoxide and purified through reversed phase column chromatography (acetonitrile/water) to afford 5-(cyclopent-1-en-1-yl)-2-cyclopropyl-4-methoxy-N-(oxazol-5-ylmethyl)benzamide. ESI MS m/z=339.3 [M+H]⁺.

### Step 2

In a 4 mL vial equipped with a stir bar, 5-(cyclopent-1-en-1-yl)-2-cyclopropyl-4-methoxy-N-(oxazol-5-ylmethyl)benzamide (51 mg, 1.0 equiv.) was dissolved in THF (1.0 mL) and water (1.0 mL). The reaction vial was purged with nitrogen and followed by addition of 10% Pd/C (80 mg, 0.5 equiv.). The mixture was degassed under vacuum and purged with hydrogen (gas) 5 times, and then the mixture was stirred under hydrogen (balloon) at 60° C. for 1 h. Reaction progress was monitored using LC-MS. Once completed, the reaction mixture was filtered through fritted funnel, washed with acetone. The filtrate was concentrated under vacuum to afford 5-cyclopentyl-2-cyclopropyl-4-methoxy-N-(oxazol-5-ylmethyl)benzamide. ESI MS m/z=341.4 [M+H]⁺.

### Step 3

In a 4 mL vial equipped with a stir bar, 5-cyclopentyl-2-cyclopropyl-4-methoxy-N-(oxazol-5-ylmethyl)benzamide (44 mg, 1.1 equiv.) and ethyl 2-amino-7-((tert-butyldimethylsilyl)oxy)-6-chlorobenzo[b]thiophene-3-carboxylate (50 mg, 1.0 equiv.) were combined neat under nitrogen atmosphere and followed by addition of dry DCE (0.6 mL). Phosphorus(V) oxychloride (27 μL, 2.5 equiv., CAS #: 10025-87-3) was added dropwise and reaction mixture was stirred for 6 h at 80° C. in a heating block. Reaction progress was monitored using LC-MS. Upon cooling to room temperature, solvent (DCE) was removed, and crude was redissolved in MeOH (0.6 mL) and followed by addition of sodium methoxide (25 wt % in MeOH, 0.13 mL, 5.0 equiv.). The reaction mixture was heated at 65° C. for 2 h in a heating block. Upon cooling to room temperature, the reaction mixture was quenched with 1N HCl, concentrated under vacuum. The crude was redissolved in 3.0 mL of dimethyl sulfoxide and purified through RPHPLC to afford 7-chloro-2-(5-cyclopentyl-2-cyclopropyl-4-methoxyphenyl)-8-hydroxy-3-(oxazol-5-ylmethyl)benzo[4,5]thieno[2,3-d]pyrimidin-4(3H)-one (13 mg, 20% yield). ESI MS m/z=550.2 [M+H]⁺.

### Synthesis of 7-chloro-2-(2-cyclopropyl-4-(oxazol-2-yl)phenyl)-8-hydroxy-3-(oxazol-5-ylmethyl)benzo[4,5]thieno[2,3-d]pyrimidin-4(3H)-one (Example 110)

### Example 110

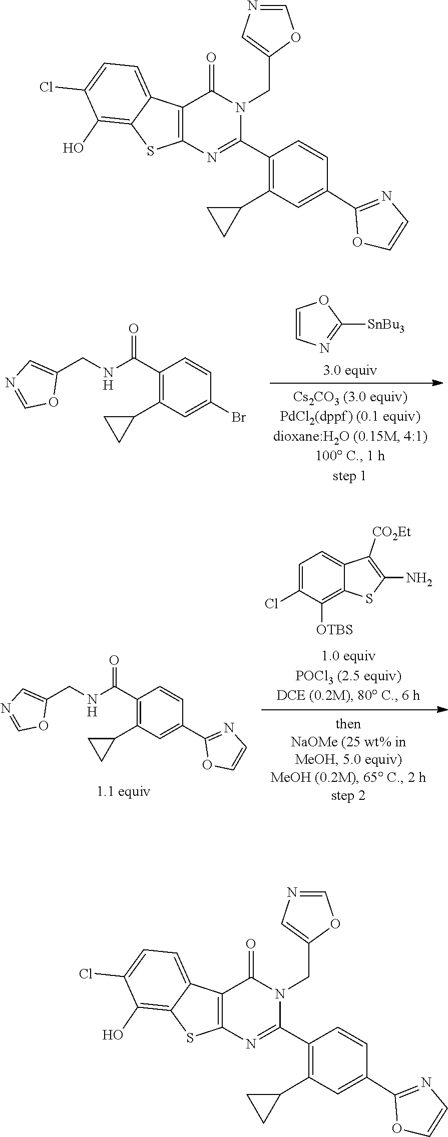

### Step 1

In a 4 mL vial equipped with a stir bar, 4-bromo-2-cyclopropyl-N-(oxazol-5-ylmethyl)benzamide (60 mg, 1.0 equiv.), 2-(tributylstannyl)oxazole (200 mg, 2.5 equiv., CAS #145214-05-7), Pd(dppf)C₁₂ (21 mg, 0.15 equiv. CAS #: 72287-26-4) and cesium carbonate (180 mg, 3.0 equiv., CAS #: 534-17-8) were combined neat under nitrogen atmosphere and followed by addition of 1,4-dioxane (1.0 mL) and water (0.25 mL). The reaction mixture was heated at 100° C. for 1 h in a heating block. Upon cooling to room temperature, the reaction mixture was concentrated and was redissolved in 3.0 mL of dimethyl sulfoxide and purified through reversed phase column chromatography (acetonitrile/water) to afford 2-cyclopropyl-4-(oxazol-2-yl)-N-(oxazol-5-ylmethyl)benzamide (42 mg, 73% yield). ESI MS m/z=310.1 [M+H]⁺.

### Step 2

In a 4 mL vial equipped with a stir bar, 2-cyclopropyl-4-(oxazol-2-yl)-N-(oxazol-5-ylmethyl)benzamide (36 mg, 1.1 equiv.) and ethyl 2-amino-7-((tert-butyldimethylsilyl)oxy)-6-chlorobenzo[b]thiophene-3-carboxylate (45 mg, 1.0 equiv.) were combined neat under nitrogen atmosphere and followed by addition of dry DCE (0.53 mL). Phosphorus(V) oxychloride (24 μL, 2.5 equiv., CAS #: 10025-87-3) was added dropwise and reaction mixture was stirred for 6 h at 80° C. in a heating block. Reaction progress was monitored using LC-MS. Upon cooling to room temperature, solvent (DCE) was removed, and crude was redissolved in MeOH (0.53 mL) and followed by addition of sodium methoxide (0.5 N in MeOH, 1.1 mL, 5.0 equiv.). The reaction mixture was heated at 65° C. for 2 h in a heating block. Upon cooling to room temperature, the reaction mixture was quenched with 1N HCl, concentrated under vacuum. The crude was redissolved in 3.0 mL of dimethyl sulfoxide, and purified through RPHPLC to afford 7-chloro-2-(2-cyclopropyl-4-(oxazol-2-yl)phenyl)-8-hydroxy-3-(oxazol-5-ylmethyl)benzo[4,5]thieno[2,3-d]pyrimidin-4(3H)-one (9.0 mg, 20% yield). ESI MS m/z=516.9 [M+H]⁺.

### Synthesis of 5-(7-chloro-8-hydroxy-3-(oxazol-5-ylmethyl)-4-oxo-3,4-dihydrobenzo[4,5]thieno[2,3-d]pyrimidin-2-yl)-4-cyclopropyl-2-methoxybenzonitrile (Example 111)

### Example 111

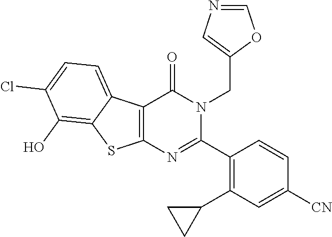 [SMILES: N#Cc1ccc(-c2nc3sc4c(O)c(Cl)ccc4c3c(=O)n2Cc2cnco2)c(C2CC2)c1]

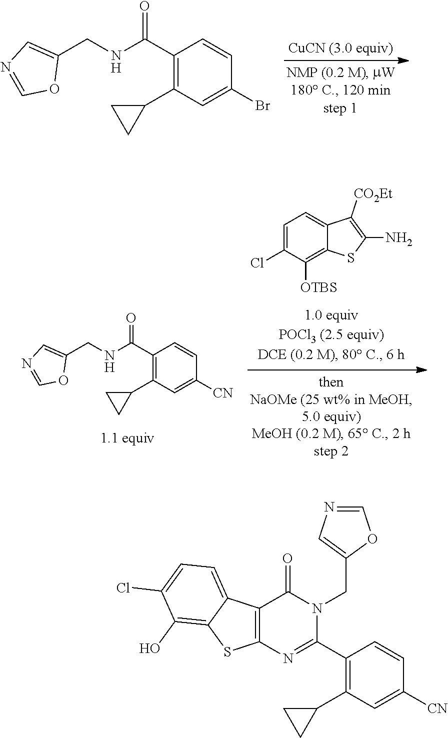 [SMILES: O=C(NCc1cnco1)c1ccc(Br)cc1C1CC1 ; CCOC(=O)c1c(N)sc2c(O[Si](C)(C)C(C)(C)C)c(Cl)ccc12 ; N#Cc1ccc(C(=O)NCc2cnco2)c(C2CC2)c1 ; N#Cc1ccc(-c2nc3sc4c(O)c(Cl)ccc4c3c(=O)n2Cc2cnco2)c(C2CC2)c1]

### Step 1

In a 2 mL microwave vial equipped with a stir bar 4-bromo-2-cyclopropyl-N-(oxazol-5-ylmethyl)benzamide (50 mg, 1.0 equiv.) and copper(I) cyanide (42 mg, 3.0 equiv., CAS #544-92-3) were combined neat under nitrogen atmosphere and followed by addition of dry NMP (0.78 mL). Reaction mixture was further flushed with nitrogen (×3) and then heated at 180° C. for 120 min under microwave condition. After completion, crude reaction mixture was directly loaded into 30 G gold C18 column and purified through reversed phase column chromatography (acetonitrile/water) to afford 4-cyano-2-cyclopropyl-N-(oxazol-5-ylmethyl)benzamide (27 mg, 54% yield). ESI MS m/z=268.0 [M+H]⁺.

### Step 2

In a 4 mL vial equipped with a stir bar, 4-cyano-2-cyclopropyl-N-(oxazol-5-ylmethyl)benzamide (27 mg, 1.1 equiv.) and ethyl 2-amino-7-((tert-butyldimethylsilyl)oxy)-6-chlorobenzo[b]thiophene-3-carboxylate (40 mg, 1.0 equiv.) were combined neat under nitrogen atmosphere and followed by addition of dry DCE (0.47 mL). Phosphorus(V) oxychloride (22 μL, 2.5 equiv., CAS #: 10025-87-3) was added dropwise and reaction mixture was stirred for 6 h at 80° C. in a heating block. Reaction progress was monitored using LC-MS. Upon cooling to room temperature, solvent (DCE) was removed, and crude was redissolved in MeOH (0.47 mL) and followed by addition of sodium methoxide (25 wt % in MeOH, 0.11 mL, 5.0 equiv.). The reaction mixture was heated at 65° C. for 2 h in a heating block. Upon cooling to room temperature, the reaction mixture was quenched with 1N HCl, concentrated under vacuum. The crude was redissolved in 3.0 mL of dimethyl sulfoxide, and purified through RPHPLC to afford 4-(7-chloro-8-hydroxy-3-(oxazol-5-ylmethyl)-4-oxo-3,4-dihydrobenzo[4,5]thieno[2,3-d]pyrimidin-2-yl)-3-cyclopropylbenzonitrile (9.5 mg, 21% yield). ESI MS m/z=475.0 [M+H]⁺.

### Synthesis of 7-chloro-2-(3-cyclopropyl-5-(methylsulfonyl)pyridin-2-yl)-8-hydroxy-3-((3-(trifluoromethyl)isoxazol-5-yl)methyl)benzo[4,5]thieno[2,3-d]pyrimidin-4(3H)-one (Example 112)

### Example 112

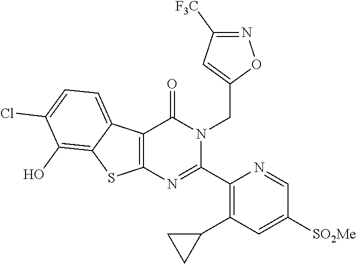 [SMILES: CS(=O)(=O)c1cnc(-c2nc3sc4c(O)c(Cl)ccc4c3c(=O)n2Cc2cc(C(F)(F)F)no2)c(C2CC2)c1]

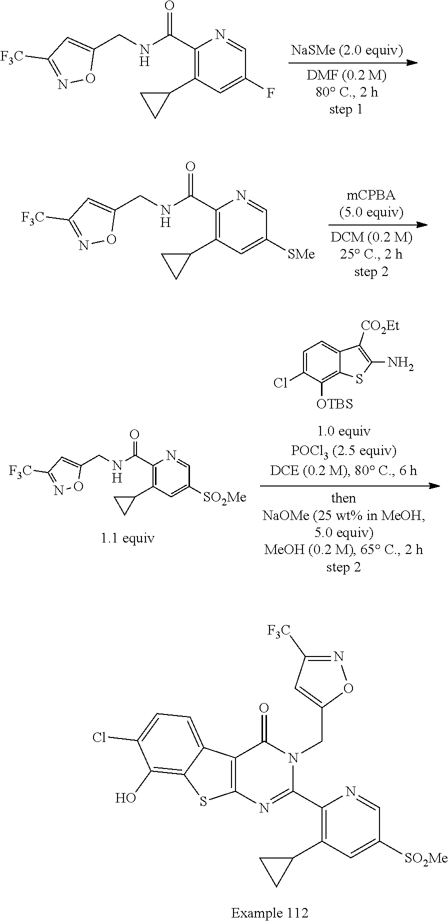 [SMILES: O=C(NCc1cc(C(F)(F)F)no1)c1ncc(F)cc1C1CC1 ; CSc1cnc(C(=O)NCc2cc(C(F)(F)F)no2)c(C2CC2)c1 ; CCOC(=O)c1c(N)sc2c(O[Si](C)(C)C(C)(C)C)c(Cl)ccc12 ; CS(=O)(=O)c1cnc(C(=O)NCc2cc(C(F)(F)F)no2)c(C2CC2)c1 ; CS(=O)(=O)c1cnc(-c2nc3sc4c(O)c(Cl)ccc4c3c(=O)n2Cc2cc(C(F)(F)F)no2)c(C2CC2)c1]

### Step 1

In a 4 mL vial equipped with a stir bar, 3-cyclopropyl-5-fluoro-N-((3-(trifluoromethyl)isoxazol-5-yl)methyl)picolinamide (50 mg, 1.0 equiv.) and sodium methanethiolate (21 mg, 2.0 equiv., CAS #5188-07-8) were combined neat under nitrogen atmosphere and followed by addition of dry DMF (0.76 mL). Reaction mixture was heated at 80° C. for 2 h in a heating block. Reaction progress was monitored using LC-MS. After completion, crude reaction mixture was directly loaded into 30 G gold C18 column and purified through reversed phase column chromatography (acetonitrile/water) to afford 3-cyclopropyl-5-(methylthio)-N-((3-(trifluoromethyl)isoxazol-5-yl)methyl)picolinamide. ESI MS m/z=358.4 [M+H]⁺.

### Step 2

In a 4 mL vial equipped with a stir bar, 3-cyclopropyl-5-(methylthio)-N-((3-(trifluoromethyl)isoxazol-5-yl)methyl)picolinamide (54 mg, 1.0 equiv.) was dissolved in dry DCM (0.76 mL) under nitrogen atmosphere at 0° C. and followed by addition of and mCPBA (130 mg, 5.0 equiv., CAS #937-14-4). Then, the ice bath was removed, and the reaction mixture was stirred at room temperature for 2 h. Reaction progress was monitored using LC-MS. After completion, crude reaction mixture was directly loaded into 30 G gold C18 column and purified through reversed phase column chromatography (acetonitrile/water) to afford 3-cyclopropyl-5-(methylsulfonyl)-N-((3-(trifluoromethyl)isoxazol-5-yl)methyl)picolinamide (36 mg, 61% yield). ESI MS m/z=390.1 [M+H]⁺.

### Step 3

In a 4 mL vial equipped with a stir bar, 3-cyclopropyl-5-(methylsulfonyl)-N-((3-(trifluoromethyl)isoxazol-5-yl)methyl)picolinamide (36 mg, 1.0 equiv.) and ethyl 2-amino-7-((tert-butyldimethylsilyl)oxy)-6-chlorobenzo[b]thiophene-3-carboxylate (40 mg, 1.0 equiv.) were combined neat under nitrogen atmosphere and followed by addition of dry DCE (0.47 mL). Phosphorus(V) oxychloride (22 μL, 2.5 equiv., CAS #: 10025-87-3) was added dropwise and reaction mixture was stirred for 6 h at 80° C. in a heating block. Reaction progress was monitored using LC-MS. Upon cooling to room temperature, solvent (DCE) was removed, and crude was redissolved in MeOH (0.47 mL) and followed by addition of sodium methoxide (25 wt % in MeOH, 0.11 mL, 5.0 equiv.). The reaction mixture was heated at 65° C. for 2 h in a heating block. Upon cooling to room temperature, the reaction mixture was quenched with 1N HCl, concentrated under vacuum. The crude was redissolved in 3.0 mL of dimethyl sulfoxide, and purified through RPHPLC to afford 7-chloro-2-(3-cyclopropyl-5-(methylsulfonyl)pyridin-2-yl)-8-hydroxy-3-((3-(trifluoromethyl)isoxazol-5-yl)methyl)benzo[4,5]thieno[2,3-d]pyrimidin-4(3H)-one (2.4 mg, 4.1% yield). ESI MS m/z=597.0 [M+H]⁺.

### Synthesis of 4-(7-chloro-8-hydroxy-4-oxo-3-((6-(trifluoromethyl)pyridin-3-yl)methyl)-3,4-dihydrobenzo[4,5]thieno[2,3-d]pyrimidin-2-yl)-3-cyclopropylbenzonitrile (Example 113)

### Example 113

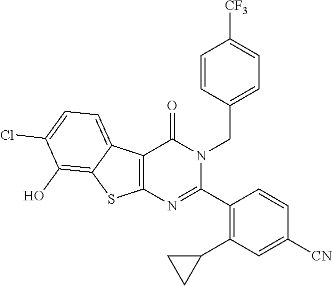 [SMILES: N#Cc1ccc(-c2nc3sc4c(O)c(Cl)ccc4c3c(=O)n2Cc2ccc(C(F)(F)F)cc2)c(C2CC2)c1]

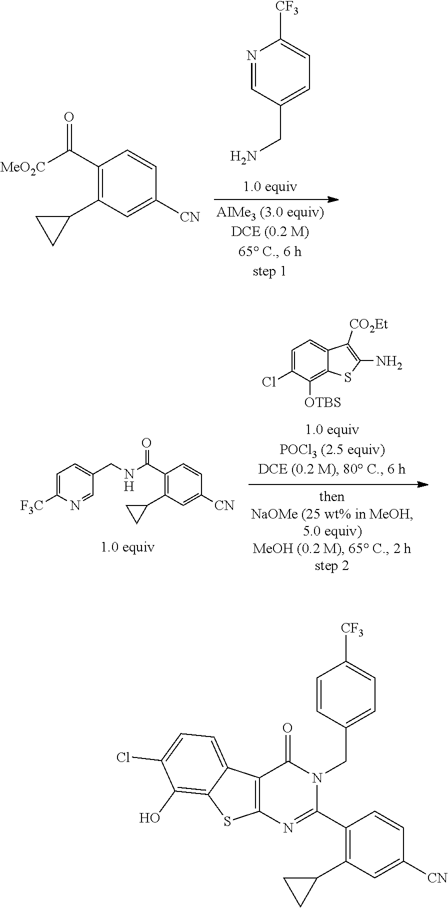 [SMILES: COC(=O)C(=O)c1ccc(C#N)cc1C1CC1 ; NCc1ccc(C(F)(F)F)nc1 ; CCOC(=O)c1c(N)sc2c(O[Si](C)(C)C(C)(C)C)c(Cl)ccc12 ; N#Cc1ccc(C(=O)NCc2ccc(C(F)(F)F)nc2)c(C2CC2)c1 ; N#Cc1ccc(-c2nc3sc4c(O)c(Cl)ccc4c3c(=O)n2Cc2ccc(C(F)(F)F)cc2)c(C2CC2)c1]

### Step 1

In a 8 mL vial equipped with a stir bar, methyl 2-(4-cyano-2-cyclopropylphenyl)-2-oxoacetate (70 mg, 1.0 equiv.) and (6-(trifluoromethyl)pyridin-3-yl)methanamine (61 mg, 1.0 equiv.) were combined neat under nitrogen atmosphere and followed by addition of dry DCE (1.7 mL). Then trimethylaluminum (2M solⁿ in Hexane, 0.52 mL, 3.0 equiv.) was added dropwise and reaction mixture was heated at 65° C. for 6 h in a heating block. Reaction progress was monitored using LC-MS. After completion, the reaction mixture was quenched by ice cold water and followed by addition of potassium sodium tartrate tetrahydrate, while placing the reaction tube in ice bath, subsequently extracted with ethyl acetate. The aqueous layer was further extracted with ethyl acetate two additional times. Then combined organic layers were washed with water (×2) and brine (×1). The organic layer was dried over sodium sulfate and concentrated under vacuum and purified through reversed phase column chromatography (acetonitrile/water) to afford 4-cyano-2-cyclopropyl-N-((6-(trifluoromethyl)pyridin-3-yl)methyl)benzamide (47 mg, 39% yield). ESI MS m/z=346.2 [M+H]⁺.

### Step 2

In a 4 mL vial equipped with a stir bar, 4-cyano-2-cyclopropyl-N-((6-(trifluoromethyl)pyridin-3-yl)methyl)benzamide (44 mg, 1.0 equiv.) and ethyl 2-amino-7-((tert-butyldimethylsilyl)oxy)-6-chlorobenzo[b]thiophene-3-carboxylate (55 mg, 1.0 equiv.) were combined neat under nitrogen atmosphere and followed by addition of dry DCE (0.64 mL). Phosphorus(V) oxychloride (30 μL, 2.5 equiv., CAS #: 10025-87-3) was added dropwise and reaction mixture was stirred for 12 h at 80° C. in a heating block. Reaction progress was monitored using LC-MS. Upon cooling to room temperature, solvent (DCE) was removed, and crude was redissolved in MeOH (0.64 mL) and followed by addition of sodium methoxide (25 wt % in MeOH, 0.15 mL, 5.0 equiv.). The reaction mixture was heated at 65° C. for 2 h in a heating block. Upon cooling to room temperature, the reaction mixture was quenched with 1N HCl, concentrated under vacuum. The crude was redissolved in 3.0 mL of dimethyl sulfoxide and purified through RPHPLC to afford 4-(7-chloro-8-hydroxy-4-oxo-3-((6-(trifluoromethyl)pyridin-3-yl)methyl)-3,4-dihydrobenzo[4,5]thieno[2,3-d]pyrimidin-2-yl)-3-cyclopropylbenzonitrile (13.4 mg, 19% yield). ESI MS m/z=552.8 [M+H]⁺.

The following compounds were prepared using a procedure analogous to that described for Example 113

| Example # | Structure |
|---|---|
| 114 | 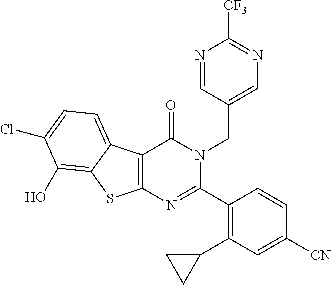 [SMILES: N#Cc1ccc(-c2nc3sc4c(O)c(Cl)ccc4c3c(=O)n2Cc2cnc(C(F)(F)F)nc2)c(C2CC2)c1] |
| 115 | 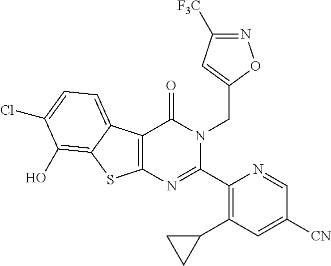 [SMILES: N#Cc1cnc(-c2nc3sc4c(O)c(Cl)ccc4c3c(=O)n2Cc2cc(C(F)(F)F)no2)c(C2CC2)c1] |

### Synthesis of 7-chloro-2-(3-cyclopropyl-5-(difluoromethyl)pyridin-2-yl)-8-hydroxy-3-((2-(trifluoromethyl)pyrimidin-5-yl)methyl)benzo[4,5]thieno[2,3-d]pyrimidin-4(3H)-one (Example 116)

### Example 116

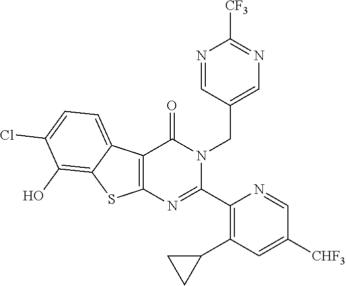

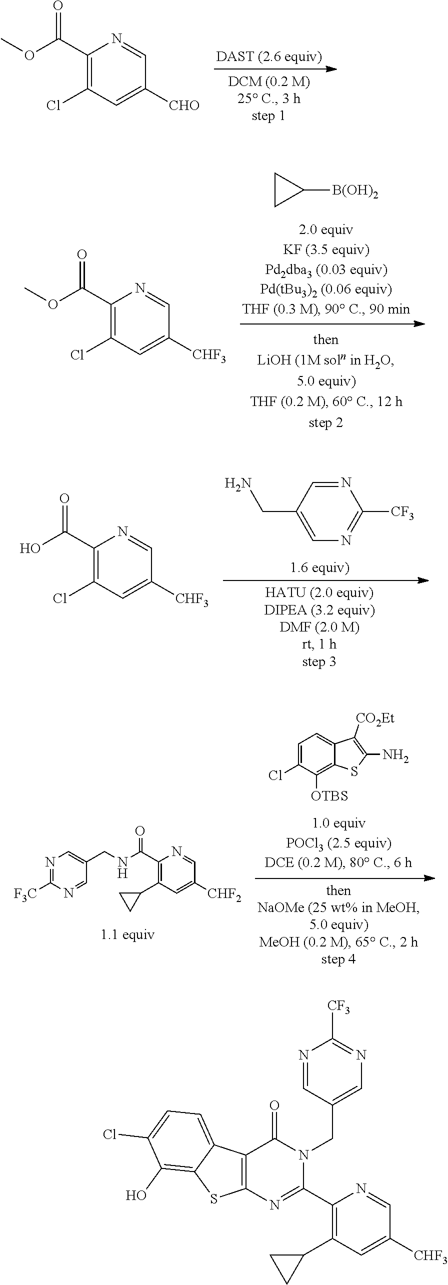

### Step 1

In a 40 mL vial equipped with a stir bar, methyl 3-chloro-5-formylpicolinate (1.0 g, 1.0 equiv., CAS #1260671-33-7) was dissolved in dry DCM (12.5 mL) at 0° C., followed by added DAST (2.1 g, 5.0 equiv., CAS #38078-09-0). Then, ice bath was removed, and reaction mixture was stirred at room temperature for 3 h. Reaction progress was monitored using LC-MS. Once completed, the reaction mixture was quenched with saturated aqueous NaHCO₃ and extracted with DCM. The organic layer was dried over sodium sulfate and concentrated under vacuum and purified through reversed phase column chromatography (acetonitrile/water) to methyl 3-chloro-5-(difluoromethyl)picolinate (840 mg, 76% yield). ESI MS m/z=221.9 [M+H]⁺.

### Step 2

In a 40 mL vial equipped with a stir bar, methyl 3-chloro-5-(difluoromethyl)picolinate (840 mg, 1.0 equiv.), cyclopropylboronic acid (651 mg, 2.0 equiv., CAS #411235-57-9), Pd₂dba₃ (104 mg, 0.03 equiv.), Pd(tBu₃)₂ (116 mg, 0.06 equiv.) and KF (771 mg, 3.5 equiv.) were combined neat under nitrogen atmosphere and followed by addition of dry THF (12.6 mL). The reaction mixture was heated at 90° C. for 90 min in a heating block. After completion, LiOH (38 mL, 10.0 equiv., 1.0 M solⁿ in H₂O) was added and the reaction mixture was heated at 60° C. for 12 h in a heating block. Upon cooling to the room temperature, the reaction mixture was quenched with 1N HCl and extracted with ethyl acetate (×3). The combined organic layer was further washed with water (×1) and brine (×1). The organic layer was dried over sodium sulfate and concentrated under vacuum. The crude residue was purified through reverse phase column chromatography (acetonitrile/water) to afford 3-cyclopropyl-5-(difluoromethyl)picolinic acid (145 mg, 18% yield). ESI MS m/z=214.0 [M+H]⁺.

### Step 3

In a 8 mL vial equipped with a stir bar, 3-cyclopropyl-5-(difluoromethyl)picolinic acid (50 mg, 1.0 equiv.), (2-(trifluoromethyl)pyrimidin-5-yl)methanamine (66 mg, 1.6 equiv), and HATU (180 mg, 2.0 equiv., CAS #148893-10-1) were combined neat under nitrogen atmosphere and followed by addition of dry DMF (1.2 mL) at room temperature. DIPEA (0.13 mL, 3.2 equiv.) was added dropwise, and reaction mixture was stirred for 1 h at 25° C. Reaction progress was monitored using LC-MS. Once completed, DCM was removed under vacuum, and crude was redissolved in 3.0 mL of dimethyl sulfoxide, and purified through reversed phase column chromatography (acetonitrile/water) to afford 3-cyclopropyl-5-(difluoromethyl)-N-((2-(trifluoromethyl)pyrimidin-5-yl)methyl)picolinamide (67 mg, 77% yield).

### Step 4

In a 40 mL vial equipped with a stir bar, 3-cyclopropyl-5-(difluoromethyl)-N-((2-(trifluoromethyl)pyrimidin-5-yl)methyl)picolinamide (67 mg, 1.1 equiv.) and ethyl 2-amino-7-((tert-butyldimethylsilyl)oxy)-6-chlorobenzo[b]thiophene-3-carboxylate (70 mg, 1.0 equiv.) were combined neat under nitrogen atmosphere and followed by addition of dry DCE (0.82 mL). Phosphorus(V) oxychloride (38 μL, 2.5 equiv., CAS #: 10025-87-3) was added dropwise and reaction mixture was stirred for 12 h at 80° C. in a heating block. Reaction progress was monitored using LC-MS. Upon cooling to room temperature, solvent (DCE) was removed, and crude was redissolved in MeOH (0.82 mL) and followed by addition of sodium methoxide (25 wt % in MeOH, 0.19 mL, 5.0 equiv.). The reaction mixture was heated at 65° C. for 2 h in a heating block. Upon cooling to room temperature, the reaction mixture was quenched with 1N HCl, concentrated under vacuum. The crude was redissolved in 3.0 mL of dimethyl sulfoxide, and purified through RPHPLC to afford 7-chloro-2-(3-cyclopropyl-5-(difluoromethyl)pyridin-2-yl)-8-hydroxy-3-((2-(trifluoromethyl)pyri mi din-5-yl)methyl)benzo[4,5]thieno[2,3-d]pyrimidin-4(3H)-one (3.5 mg, 3.5% yield). ESI MS m/z=579.9 [M+H]⁺.

The following compounds examples 117˜187 were prepared using procedures similar to that described above.

| Example # | Structure |
|---|---|
| 117 | 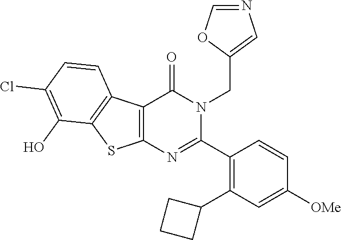 [SMILES: COc1ccc(-c2nc3sc4c(O)c(Cl)ccc4c3c(=O)n2Cc2cnco2)c(C2CCC2)c1] |
| 118 | 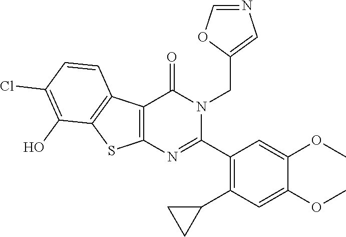 [SMILES: COc1cc(-c2nc3sc4c(O)c(Cl)ccc4c3c(=O)n2Cc2cnco2)c(C2CC2)cc1OC] |
| 119 | 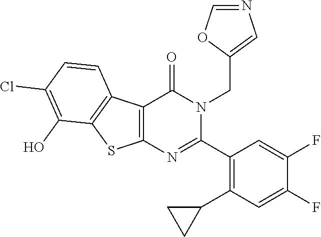 [SMILES: O=c1c2c(nc(-c3cc(F)c(F)cc3C3CC3)n1Cc1cnco1)sc1c(O)c(Cl)ccc12] |
| 120 | 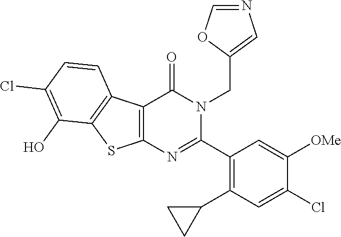 [SMILES: COc1cc(-c2nc3sc4c(O)c(Cl)ccc4c3c(=O)n2Cc2cnco2)c(C2CC2)cc1Cl] |
| 121 | 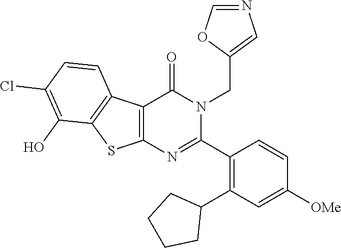 [SMILES: COc1ccc(-c2nc3sc4c(O)c(Cl)ccc4c3c(=O)n2Cc2cnco2)c(C2CCCC2)c1] |
| 122 | 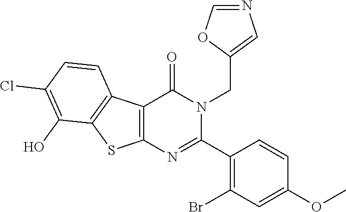 [SMILES: COc1ccc(-c2nc3sc4c(O)c(Cl)ccc4c3c(=O)n2Cc2cnco2)c(Br)c1] |
| 123 | 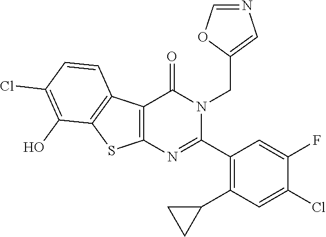 [SMILES: O=c1c2c(nc(-c3cc(F)c(Cl)cc3C3CC3)n1Cc1cnco1)sc1c(O)c(Cl)ccc12] |
| 124 | 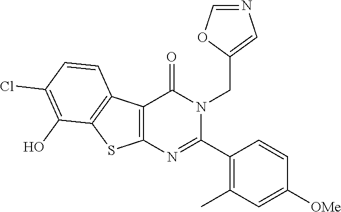 [SMILES: COc1ccc(-c2nc3sc4c(O)c(Cl)ccc4c3c(=O)n2Cc2cnco2)c(C)c1] |
| 125 | 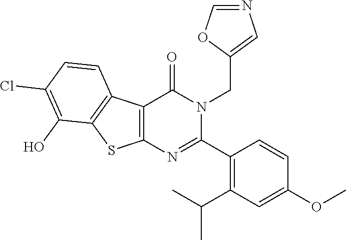 [SMILES: COc1ccc(-c2nc3sc4c(O)c(Cl)ccc4c3c(=O)n2Cc2cnco2)c(C(C)C)c1] |
| 126 | 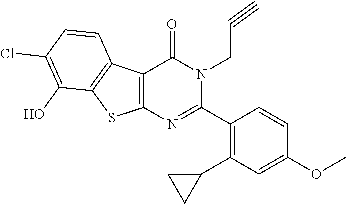 [SMILES: C#CCn1c(-c2ccc(OC)cc2C2CC2)nc2sc3c(O)c(Cl)ccc3c2c1=O] |
| 127 | 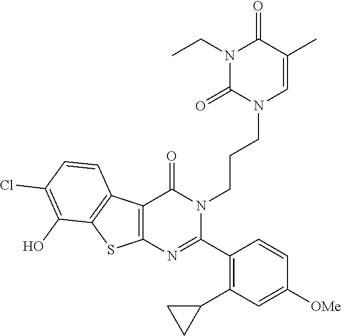 [SMILES: CCn1c(=O)c(C)cn(CCCn2c(-c3ccc(OC)cc3C3CC3)nc3sc4c(O)c(Cl)ccc4c3c2=O)c1=O] |
| 128 | 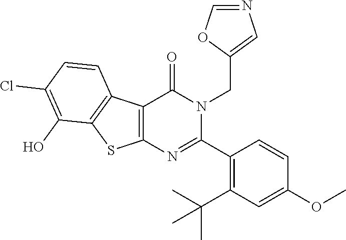 [SMILES: COc1ccc(-c2nc3sc4c(O)c(Cl)ccc4c3c(=O)n2Cc2cnco2)c(C(C)(C)C)c1] |
| 129 | 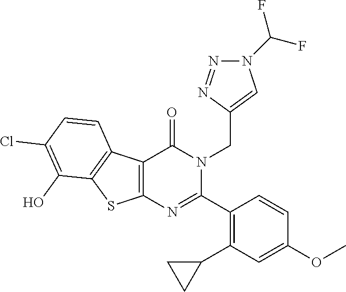 [SMILES: COc1ccc(-c2nc3sc4c(O)c(Cl)ccc4c3c(=O)n2Cc2cn(C(F)F)nn2)c(C2CC2)c1] |
| 130 | 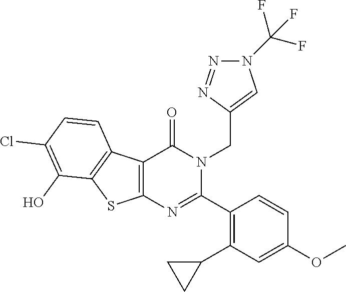 [SMILES: COc1ccc(-c2nc3sc4c(O)c(Cl)ccc4c3c(=O)n2Cc2cn(C(F)(F)F)nn2)c(C2CC2)c1] |
| 131 | 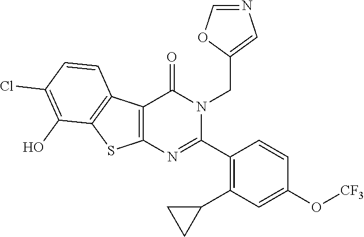 [SMILES: O=c1c2c(nc(-c3ccc(OC(F)(F)F)cc3C3CC3)n1Cc1cnco1)sc1c(O)c(Cl)ccc12] |
| 132 | 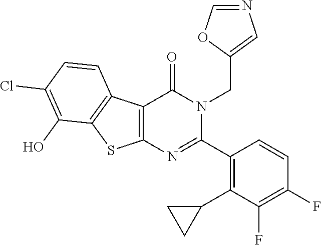 [SMILES: O=c1c2c(nc(-c3ccc(F)c(F)c3C3CC3)n1Cc1cnco1)sc1c(O)c(Cl)ccc12] |
| 133 | 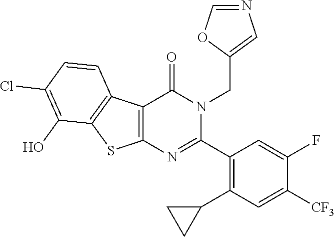 [SMILES: O=c1c2c(nc(-c3cc(F)c(C(F)(F)F)cc3C3CC3)n1Cc1cnco1)sc1c(O)c(Cl)ccc12] |
| 134 | 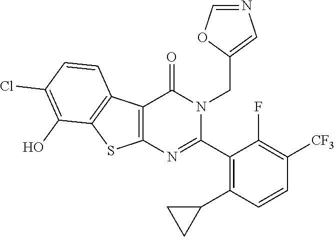 [SMILES: O=c1c2c(nc(-c3c(C4CC4)ccc(C(F)(F)F)c3F)n1Cc1cnco1)sc1c(O)c(Cl)ccc12] |
| 135 | 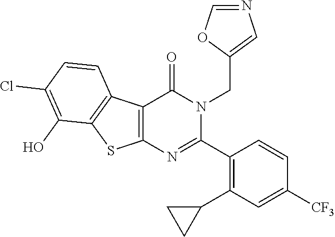 [SMILES: O=c1c2c(nc(-c3ccc(C(F)(F)F)cc3C3CC3)n1Cc1cnco1)sc1c(O)c(Cl)ccc12] |
| 136 | 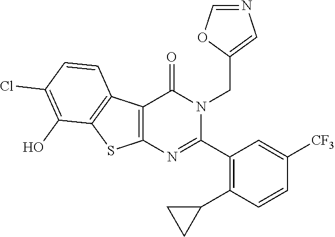 [SMILES: O=c1c2c(nc(-c3cc(C(F)(F)F)ccc3C3CC3)n1Cc1cnco1)sc1c(O)c(Cl)ccc12] |
| 137 | 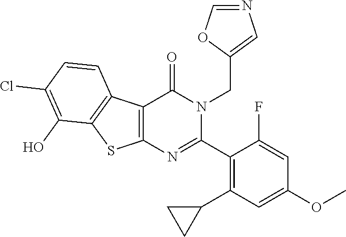 [SMILES: COc1cc(F)c(-c2nc3sc4c(O)c(Cl)ccc4c3c(=O)n2Cc2cnco2)c(C2CC2)c1] |
| 138 | 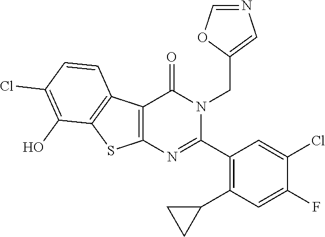 [SMILES: O=c1c2c(nc(-c3cc(Cl)c(F)cc3C3CC3)n1Cc1cnco1)sc1c(O)c(Cl)ccc12] |
| 139 | 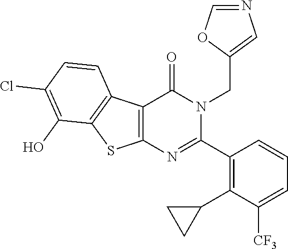 [SMILES: O=c1c2c(nc(-c3cccc(C(F)(F)F)c3C3CC3)n1Cc1cnco1)sc1c(O)c(Cl)ccc12] |
| 140 | 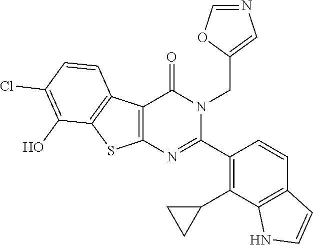 [SMILES: O=c1c2c(nc(-c3ccc4cc[nH]c4c3C3CC3)n1Cc1cnco1)sc1c(O)c(Cl)ccc12] |
| 141 | 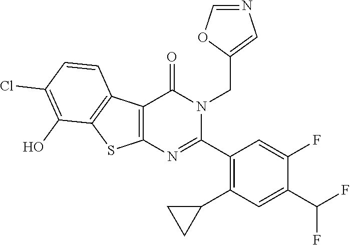 [SMILES: O=c1c2c(nc(-c3cc(F)c(C(F)F)cc3C3CC3)n1Cc1cnco1)sc1c(O)c(Cl)ccc12] |
| 142 | 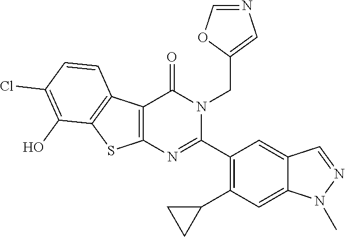 [SMILES: Cn1ncc2cc(-c3nc4sc5c(O)c(Cl)ccc5c4c(=O)n3Cc3cnco3)c(C3CC3)cc21] |
| 143 | 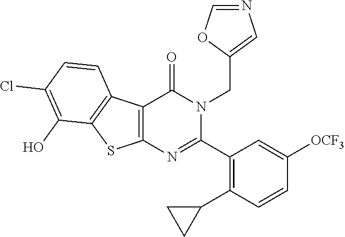 [SMILES: O=c1c2c(nc(-c3cc(OC(F)(F)F)ccc3C3CC3)n1Cc1cnco1)sc1c(O)c(Cl)ccc12] |
| 144 | 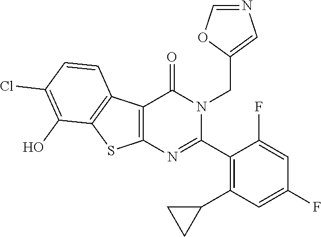 [SMILES: O=c1c2c(nc(-c3c(F)cc(F)cc3C3CC3)n1Cc1cnco1)sc1c(O)c(Cl)ccc12] |
| 145 | 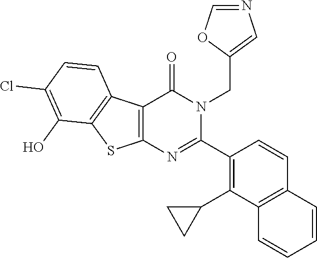 [SMILES: O=c1c2c(nc(-c3ccc4ccccc4c3C3CC3)n1Cc1cnco1)sc1c(O)c(Cl)ccc12] |
| 146 | 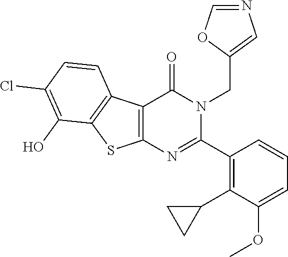 [SMILES: COc1cccc(-c2nc3sc4c(O)c(Cl)ccc4c3c(=O)n2Cc2cnco2)c1C1CC1] |
| 147 | 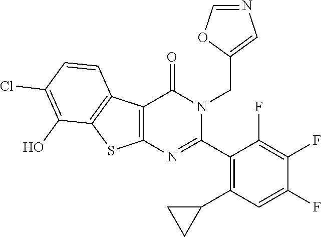 [SMILES: O=c1c2c(nc(-c3c(C4CC4)cc(F)c(F)c3F)n1Cc1cnco1)sc1c(O)c(Cl)ccc12] |
| 148 | 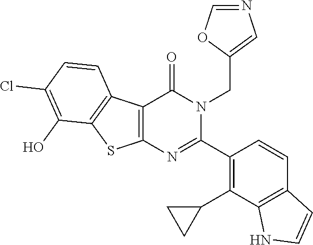 [SMILES: O=c1c2c(nc(-c3ccc4cc[nH]c4c3C3CC3)n1Cc1cnco1)sc1c(O)c(Cl)ccc12] |
| 149 | 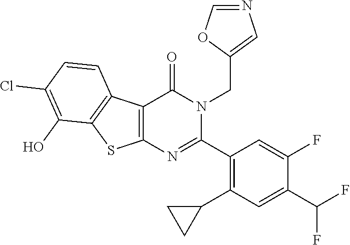 [SMILES: O=c1c2c(nc(-c3cc(F)c(C(F)F)cc3C3CC3)n1Cc1cnco1)sc1c(O)c(Cl)ccc12] |
| 150 | 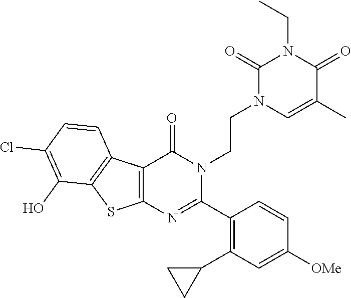 [SMILES: CCn1c(=O)c(C)cn(CCn2c(-c3ccc(OC)cc3C3CC3)nc3sc4c(O)c(Cl)ccc4c3c2=O)c1=O] |
| 151 | 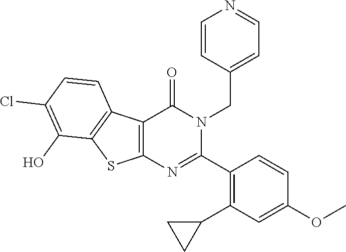 [SMILES: COc1ccc(-c2nc3sc4c(O)c(Cl)ccc4c3c(=O)n2Cc2ccncc2)c(C2CC2)c1] |
| 152 | 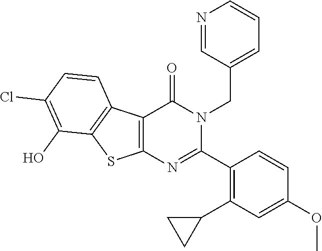 [SMILES: COc1ccc(-c2nc3sc4c(O)c(Cl)ccc4c3c(=O)n2Cc2cccnc2)c(C2CC2)c1] |
| 153 | 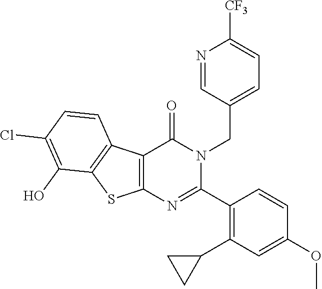 [SMILES: COc1ccc(-c2nc3sc4c(O)c(Cl)ccc4c3c(=O)n2Cc2ccc(C(F)(F)F)nc2)c(C2CC2)c1] |
| 154 | 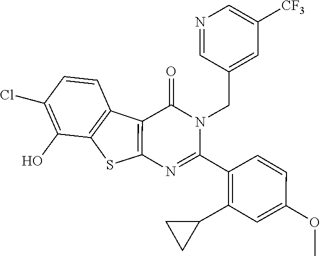 [SMILES: COc1ccc(-c2nc3sc4c(O)c(Cl)ccc4c3c(=O)n2Cc2cncc(C(F)(F)F)c2)c(C2CC2)c1] |
| 155 | 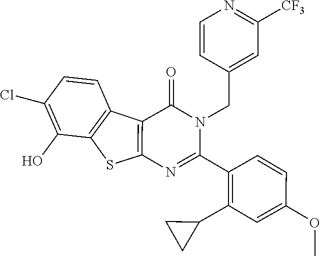 [SMILES: COc1ccc(-c2nc3sc4c(O)c(Cl)ccc4c3c(=O)n2Cc2ccnc(C(F)(F)F)c2)c(C2CC2)c1] |
| 156 | 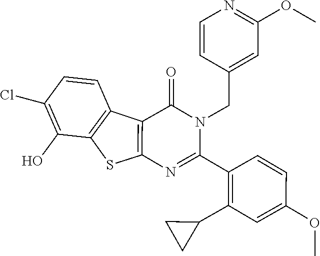 [SMILES: COc1ccc(-c2nc3sc4c(O)c(Cl)ccc4c3c(=O)n2Cc2ccnc(OC)c2)c(C2CC2)c1] |
| 157 | 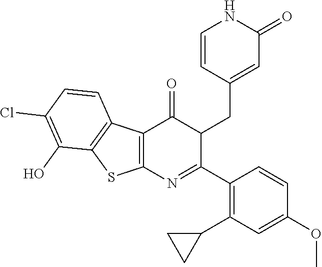 [SMILES: COc1ccc(C2=Nc3sc4c(O)c(Cl)ccc4c3C(=O)C2Cc2cc[nH]c(=O)c2)c(C2CC2)c1] |
| 158 | 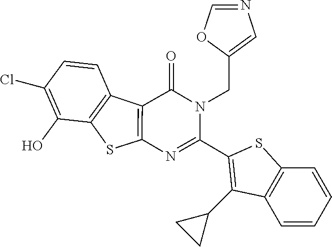 [SMILES: O=c1c2c(nc(-c3sc4ccccc4c3C3CC3)n1Cc1cnco1)sc1c(O)c(Cl)ccc12] |
| 159 | 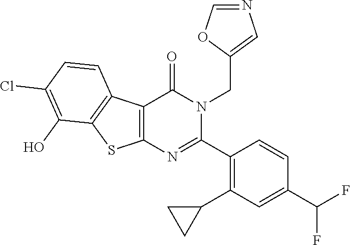 [SMILES: O=c1c2c(nc(-c3ccc(C(F)F)cc3C3CC3)n1Cc1cnco1)sc1c(O)c(Cl)ccc12] |
| 160 | 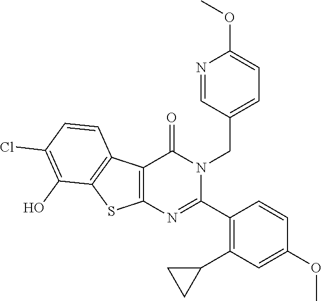 [SMILES: COc1ccc(-c2nc3sc4c(O)c(Cl)ccc4c3c(=O)n2Cc2ccc(OC)nc2)c(C2CC2)c1] |
| 161 | 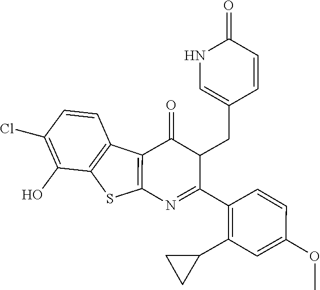 [SMILES: COc1ccc(C2=Nc3sc4c(O)c(Cl)ccc4c3C(=O)C2Cc2ccc(=O)[nH]c2)c(C2CC2)c1] |
| 162 | 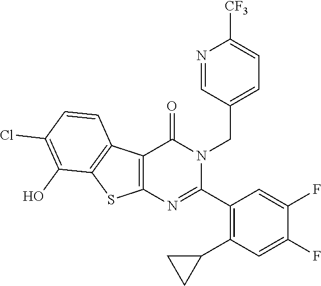 [SMILES: O=c1c2c(nc(-c3cc(F)c(F)cc3C3CC3)n1Cc1ccc(C(F)(F)F)nc1)sc1c(O)c(Cl)ccc12] |
| 163 | 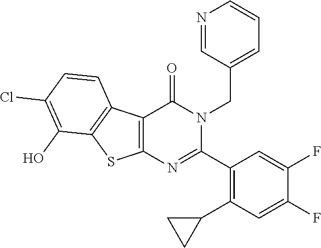 [SMILES: O=c1c2c(nc(-c3cc(F)c(F)cc3C3CC3)n1Cc1cccnc1)sc1c(O)c(Cl)ccc12] |
| 164 | 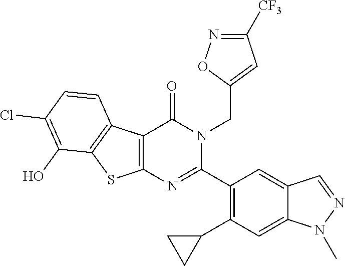 [SMILES: Cn1ncc2cc(-c3nc4sc5c(O)c(Cl)ccc5c4c(=O)n3Cc3cc(C(F)(F)F)no3)c(C3CC3)cc21] |
| 165 | 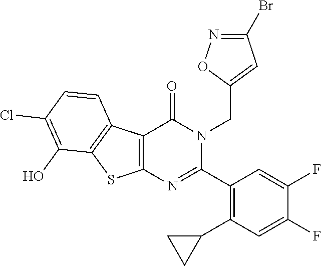 [SMILES: O=c1c2c(nc(-c3cc(F)c(F)cc3C3CC3)n1Cc1cc(Br)no1)sc1c(O)c(Cl)ccc12] |
| 166 | 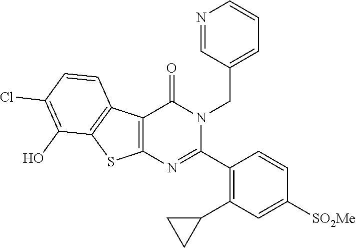 [SMILES: CS(=O)(=O)c1ccc(-c2nc3sc4c(O)c(Cl)ccc4c3c(=O)n2Cc2cccnc2)c(C2CC2)c1] |
| 167 | 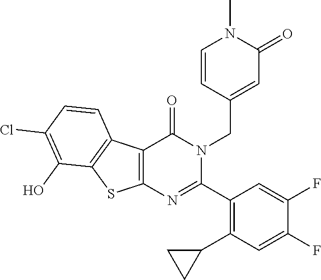 [SMILES: Cn1ccc(Cn2c(-c3cc(F)c(F)cc3C3CC3)nc3sc4c(O)c(Cl)ccc4c3c2=O)cc1=O] |
| 168 | 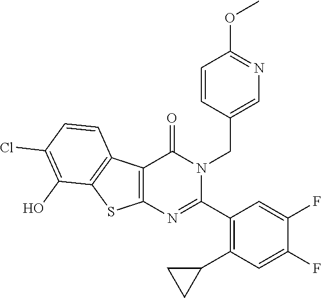 [SMILES: COc1ccc(Cn2c(-c3cc(F)c(F)cc3C3CC3)nc3sc4c(O)c(Cl)ccc4c3c2=O)cn1] |
| 169 | 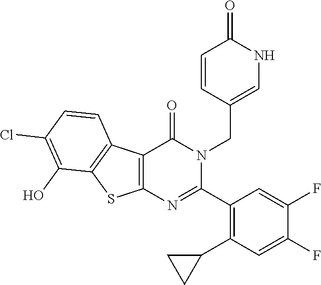 [SMILES: O=c1ccc(Cn2c(-c3cc(F)c(F)cc3C3CC3)nc3sc4c(O)c(Cl)ccc4c3c2=O)c[nH]1] |
| 170 | 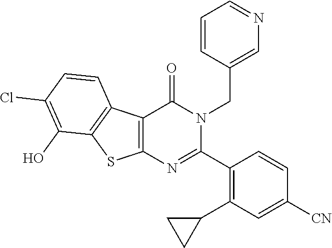 [SMILES: N#Cc1ccc(-c2nc3sc4c(O)c(Cl)ccc4c3c(=O)n2Cc2cccnc2)c(C2CC2)c1] |
| 171 | 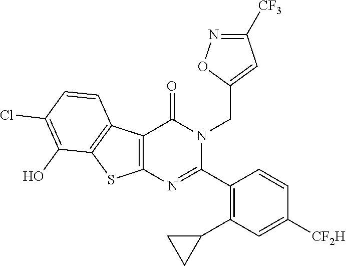 [SMILES: O=c1c2c(nc(-c3ccc(C(F)F)cc3C3CC3)n1Cc1cc(C(F)(F)F)no1)sc1c(O)c(Cl)ccc12] |
| 172 | 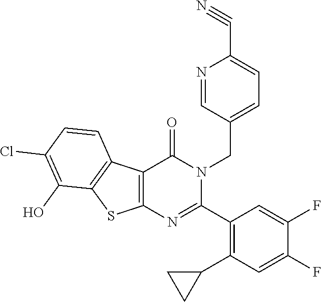 [SMILES: N#Cc1ccc(Cn2c(-c3cc(F)c(F)cc3C3CC3)nc3sc4c(O)c(Cl)ccc4c3c2=O)cn1] |
| 173 | 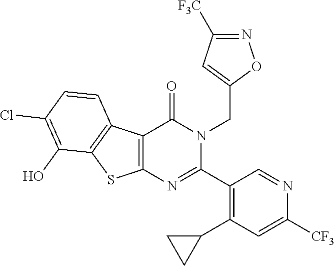 [SMILES: O=c1c2c(nc(-c3cnc(C(F)(F)F)cc3C3CC3)n1Cc1cc(C(F)(F)F)no1)sc1c(O)c(Cl)ccc12] |
| 174 | 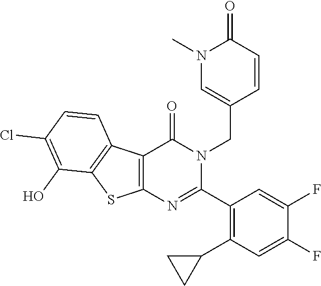 [SMILES: Cn1cc(Cn2c(-c3cc(F)c(F)cc3C3CC3)nc3sc4c(O)c(Cl)ccc4c3c2=O)ccc1=O] |
| 175 | 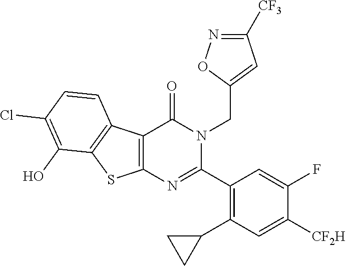 [SMILES: O=c1c2c(nc(-c3cc(F)c(C(F)F)cc3C3CC3)n1Cc1cc(C(F)(F)F)no1)sc1c(O)c(Cl)ccc12] |
| 176 | 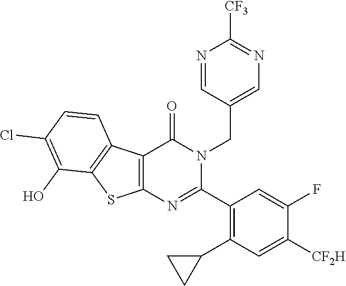 [SMILES: O=c1c2c(nc(-c3cc(F)c(C(F)F)cc3C3CC3)n1Cc1cnc(C(F)(F)F)nc1)sc1c(O)c(Cl)ccc12] |
| 177 | 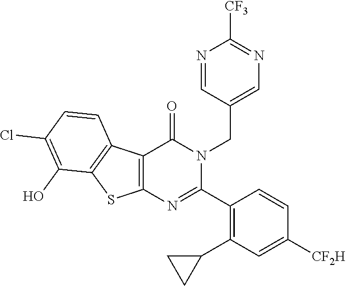 [SMILES: O=c1c2c(nc(-c3ccc(C(F)F)cc3C3CC3)n1Cc1cnc(C(F)(F)F)nc1)sc1c(O)c(Cl)ccc12] |
| 178 | 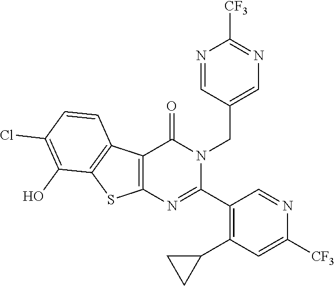 [SMILES: O=c1c2c(nc(-c3cnc(C(F)(F)F)cc3C3CC3)n1Cc1cnc(C(F)(F)F)nc1)sc1c(O)c(Cl)ccc12] |
| 179 | 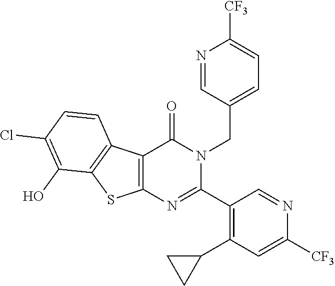 [SMILES: O=c1c2c(nc(-c3cnc(C(F)(F)F)cc3C3CC3)n1Cc1ccc(C(F)(F)F)nc1)sc1c(O)c(Cl)ccc12] |
| 180 | 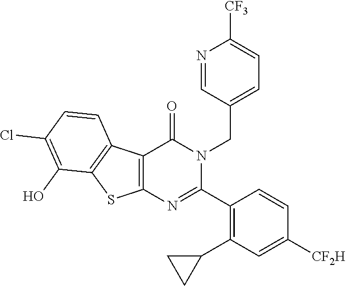 [SMILES: O=c1c2c(nc(-c3ccc(C(F)F)cc3C3CC3)n1Cc1ccc(C(F)(F)F)nc1)sc1c(O)c(Cl)ccc12] |
| 181 | 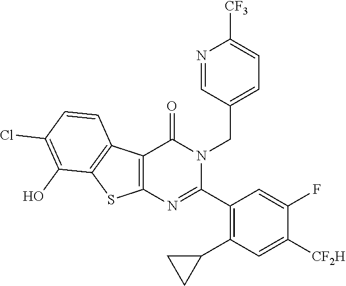 [SMILES: O=c1c2c(nc(-c3cc(F)c(C(F)F)cc3C3CC3)n1Cc1ccc(C(F)(F)F)nc1)sc1c(O)c(Cl)ccc12] |
| 182 | 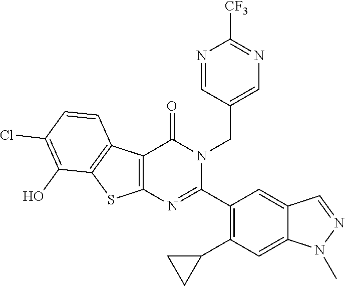 [SMILES: Cn1ncc2cc(-c3nc4sc5c(O)c(Cl)ccc5c4c(=O)n3Cc3cnc(C(F)(F)F)nc3)c(C3CC3)cc21] |
| 183 | 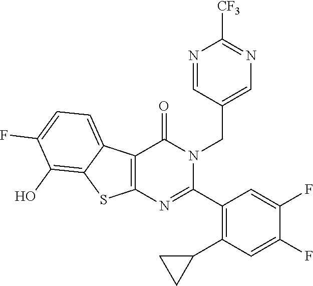 [SMILES: O=c1c2c(nc(-c3cc(F)c(F)cc3C3CC3)n1Cc1cnc(C(F)(F)F)nc1)sc1c(O)c(F)ccc12] |
| 184 | 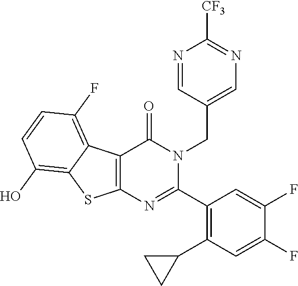 [SMILES: O=c1c2c(nc(-c3cc(F)c(F)cc3C3CC3)n1Cc1cnc(C(F)(F)F)nc1)sc1c(O)ccc(F)c12] |
| 185 | 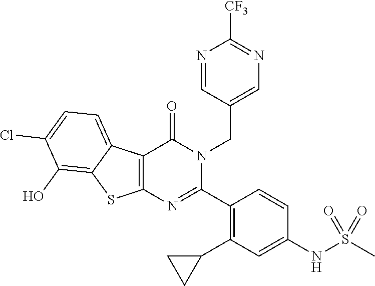 [SMILES: CS(=O)(=O)Nc1ccc(-c2nc3sc4c(O)c(Cl)ccc4c3c(=O)n2Cc2cnc(C(F)(F)F)nc2)c(C2CC2)c1] |
| 186 | 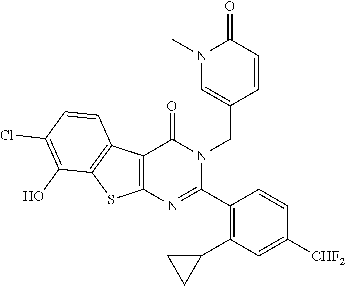 [SMILES: Cn1cc(Cn2c(-c3ccc(C(F)F)cc3C3CC3)nc3sc4c(O)c(Cl)ccc4c3c2=O)ccc1=O] |
| 187 | 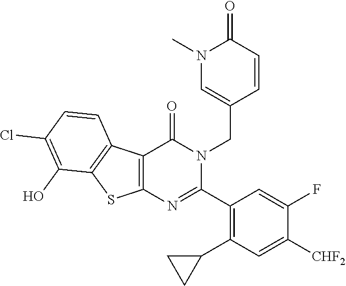 [SMILES: Cn1cc(Cn2c(-c3cc(F)c(C(F)F)cc3C3CC3)nc3sc4c(O)c(Cl)ccc4c3c2=O)ccc1=O] |

### Synthesis of 7-chloro-2-(2-cyclopropyl-4-methoxyphenyl)-8-hydroxy-3-(2-(oxazol-5-yl)ethyl)benzo[4,5]thieno[2,3-d]pyrimidin-4(3H)-one (Example 188)

### Example 188

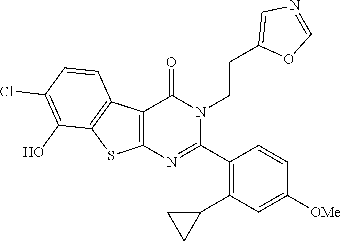 [SMILES: COc1ccc(-c2nc3sc4c(O)c(Cl)ccc4c3c(=O)n2CCc2cnco2)c(C2CC2)c1]

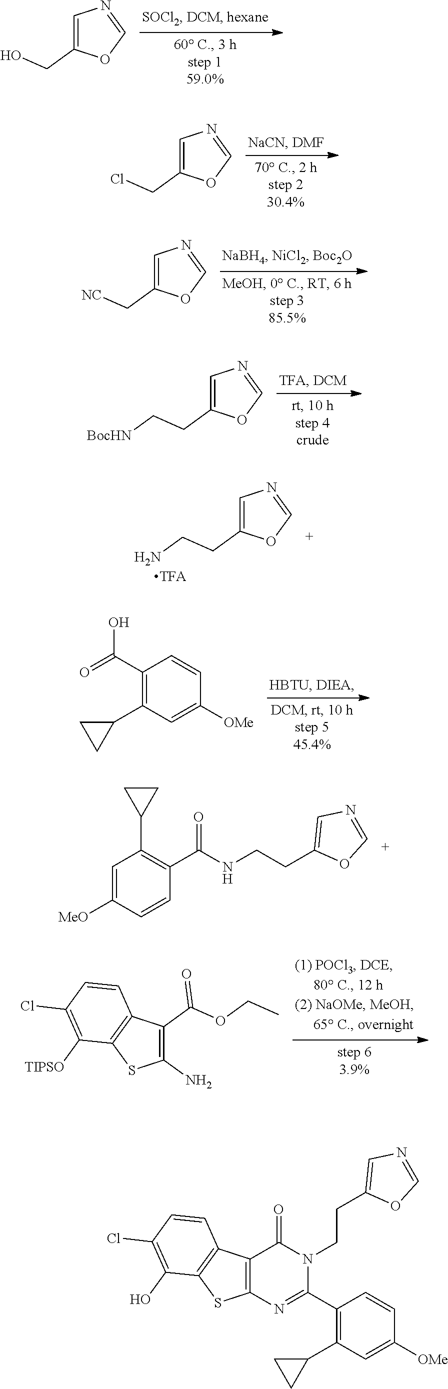

To a solution of oxazol-5-ylmethanol (2.0 g, 20.2 mmol, 1.0 eq) in DCM (6.0 mL) and hexane (6.0 mL) was added dropwise of SOCl₂ (3.6 g, 30.3 mmol, 1.5 eq) at 0° C., the reaction mixture was stirred at 60° C. for 3 h. After completion of the reaction, the reaction mixture was neutralized with saturated NaHCO₃ solution (80 mL) and extracted with ether (50 mL×2). The combined organic extracts were dried over sodium sulfate, filtered and concentrated in vacuo to give 5-(chloromethyl)oxazole (1.4 g, 59.0%) as a yellow oil. ¹H NMR (300 MHz, CDCl₃): δ 7.87 (s, 1H), 7.08 (s, 1H), 4.60 (s, 2H).

To a solution of 5-(chloromethyl)oxazole (500 mg, 4.3 mmol, 1.0 eq) in DMF (5.0 mL) was added NaCN (730 mg, 14.9 mmol, 3.5 eq), the reaction mixture was stirred at 70° C. for 2 h. To the mixture were added water (10 mL) and brine (14 mL). The mixture was extracted with Et₂O (40 mL×5). The organic phase was dried, filtered, concentrated and the residue was purified by silica gel column chromatography (Petroleum ether:Et₂O=1:1) to give 2-(oxazol-5-yl)acetonitrile (140 mg, 30.4%) as a yellow solid. ¹H NMR (300 MHz, CDCl₃): δ 7.88 (s, 1H), 7.09 (s, 1H), 3.84 (d, J=0.9 Hz, 2H).

To a stirred mixture of 2-(oxazol-5-yl)acetonitrile (100 mg, 0.92 mmol, 1.0 eq), NiCl₂ (144 mg, 1.1 mmol, 1.2 eq) and NaBH₄ (42 mg, 1.1 mmol, 1.2 eq) in MeOH (2 mL) was added Boc₂O (808 mg, 3.70 mmol, 4.0 eq), the reaction mixture was stirred at room temperature for 6 h. The mixture was quenched with water, extracted with EtOAc (30 mL×2), the organic phase was dried, filtered, concentrated and purified by silica gel column chromatography (EtOAc) to give tert-butyl (2-(oxazol-5-yl)ethyl)carbamate (168 mg, yield: 85.5%) as a white solid. ¹H NMR (300 MHz, CDCl₃): δ 7.80 (s, 1H), 6.84 (s, 1H), 4.65 (s, 1H), 3.46-3.34 (m, 2H), 2.93-2.82 (m, 2H), 1.44 (s, 9H).

To a solution of tert-butyl (2-(oxazol-5-yl)ethyl)carbamate (0.15 g, 0.70 mmol, 1.0 eq) in DCM (4.0 mL) was added TFA (5 mL), the reaction mixture was stirred at room temperature for 10 h. The mixture was evaporated to afford 2-(oxazol-5-yl)ethan-1-amine TFA salt (169 mg, crude) as a white solid. ¹H NMR (400 MHz, DMSO-d₆): δ 8.29 (s, 1H), 7.89 (brs, 3H), 7.02 (s, 1H), 3.14-3.04 (m, 2H), 3.02-2.92 (m, 2H).

To a solution of 2-(oxazol-5-yl)ethan-1-amine TFA salt (169 mg, 0.81 mmol, 1.2 eq), HBTU (641 mg, 1.69 mmol, 2.5 eq) and DIEA (0.26 g, 2.03 mmol, 3.0 eq) in DCM (1.0 mL) was added 2-cyclopropyl-4-methoxybenzoic acid (130 mg, 0.68 mmol, 1.0 eq) at room temperature, the reaction mixture was stirred at room temperature for 10 h. The reaction mixture was quenched with water (10 mL), extracted with ethyl acetate (10 mL×4). The mixture was evaporated to afford the crude product. The crude product was purified by column chromatography on silica gel (EtOAc) to give the 2-cyclopropyl-4-methoxy-N-(2-(oxazol-5-yl)ethyl)benzamide (88 mg, 45.4%) as a yellow solid. ¹H NMR (400 MHz, DMSO-d₆): δ 8.33-8.26 (m, 2H), 7.23-7.11 (m, 1H), 6.94 (s, 1H), 6.78-6.68 (m, 1H), 6.37 (d, J=2.4 Hz, 1H), 3.79 (s, 3H), 3.72-3.62 (m, 2H), 2.98 (t, J=6.8 Hz, 2H), 2.12-1.98 (m, 1H), 0.96-0.84 (m, 2H), 0.74-0.64 (m, 2H). To a solution of 2-cyclopropyl-4-methoxy-N-(2-(oxazol-5-yl)ethyl)benzamide (88 mg, 0.31 mmol, 1.3 eq) and ethyl 2-amino-6-chloro-7-((triisopropylsilyl)oxy)benzo[b]thiophene-3-carboxylate (100 mg, 0.23 mmol, 1.0 eq) in DCE (2.0 mL) was added POCl₃ (90 mg, 0.59 mmol, 2.5 eq) at 0° C. under N₂, the mixture was stirred at 80° C. for 12 h. The mixture was evaporated. To a solution of the mixture in methanol (2.0 mL) was added 30% NaOMe in MeOH (423 mg, 2.34 mmol, 10 eq) at room temperature. The mixture was stirred at 65° C. overnight. The pH was adjusted to 3 by aq. Citric acid. The aqueous layer was extracted with ethyl acetate (10 mL×2). The Organic phase was concentrated and purified by column chromatography on silica gel (CH₂Cl₂:MeOH=20:1 to 10:1) to give 7-chloro-2-(2-cyclopropyl-4-methoxyphenyl)-8-hydroxy-3-(2-(oxazol-5-yl)ethyl)benzo[4,5]thieno[2,3-d]pyrimidin-4(3H)-one (4.5 mg, 3.9%) as a white solid. LC-MS (M+H)+: m/z=493.8. ¹H NMR (400 MHz, DMSO-d₆): δ 10.71 (s, 1H), 8.16 (s, 1H), 8.06-8.01 (m, 1H), 7.58 (d, J=8.4 Hz, 1H), 7.13 (d, J=8.4 Hz, 1H), 6.86-6.82 (m, 1H), 6.78 (s, 1H), 6.48 (d, J=2.0 Hz, 1H), 4.47-4.38 (m, 1H), 3.96-3.87 (m, 1H), 3.81 (s, 3H), 3.02 (t, J=7.2 Hz, 2H), 1.62-1.57 (m, 1H), 0.87-0.81 (m, 3H), 0.72-0.67 (m, 1H).

The following compounds examples 189-196, 198-199, 203˜204, and 206 were prepared using procedures similar to example 188.

| Example # | Structure |
|---|---|
| 189 | 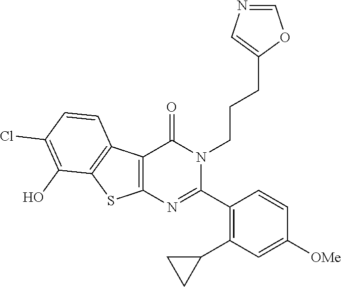 [SMILES: COc1ccc(-c2nc3sc4c(O)c(Cl)ccc4c3c(=O)n2CCCc2cnco2)c(C2CC2)c1] |
| 190 | 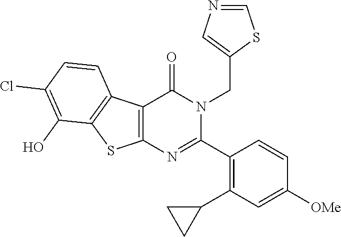 [SMILES: COc1ccc(-c2nc3sc4c(O)c(Cl)ccc4c3c(=O)n2Cc2cncs2)c(C2CC2)c1] |
| 191 | 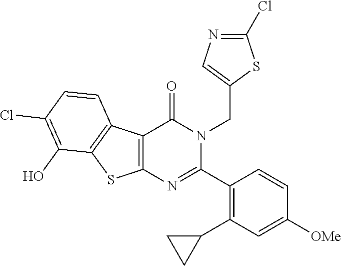 [SMILES: COc1ccc(-c2nc3sc4c(O)c(Cl)ccc4c3c(=O)n2Cc2cnc(Cl)s2)c(C2CC2)c1] |
| 192 | 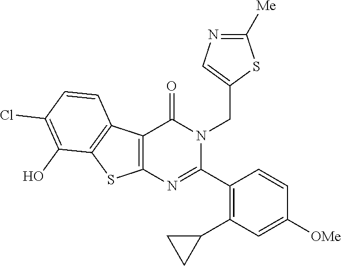 [SMILES: COc1ccc(-c2nc3sc4c(O)c(Cl)ccc4c3c(=O)n2Cc2cnc(C)s2)c(C2CC2)c1] |
| 193 | 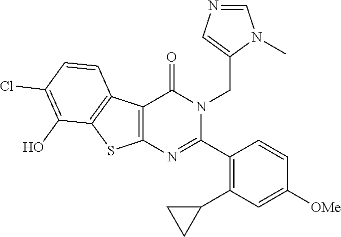 [SMILES: COc1ccc(-c2nc3sc4c(O)c(Cl)ccc4c3c(=O)n2Cc2cncn2C)c(C2CC2)c1] |
| 194 | 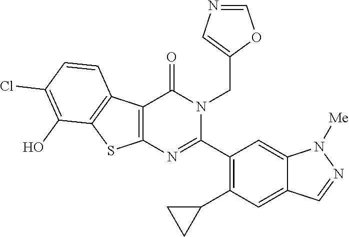 [SMILES: Cn1ncc2cc(C3CC3)c(-c3nc4sc5c(O)c(Cl)ccc5c4c(=O)n3Cc3cnco3)cc21] |
| 195 | 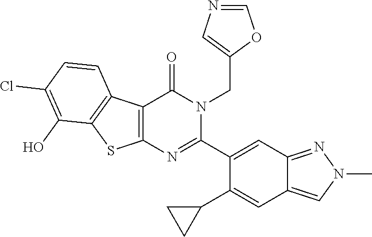 [SMILES: Cn1cc2cc(C3CC3)c(-c3nc4sc5c(O)c(Cl)ccc5c4c(=O)n3Cc3cnco3)cc2n1] |
| 196 | 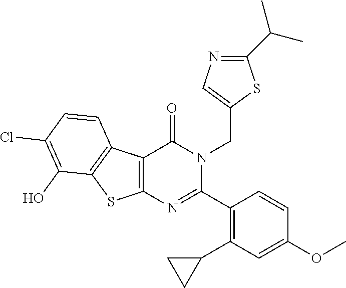 [SMILES: COc1ccc(-c2nc3sc4c(O)c(Cl)ccc4c3c(=O)n2Cc2cnc(C(C)C)s2)c(C2CC2)c1] |
| 198 | 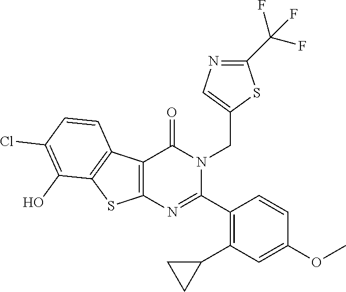 [SMILES: COc1ccc(-c2nc3sc4c(O)c(Cl)ccc4c3c(=O)n2Cc2cnc(C(F)(F)F)s2)c(C2CC2)c1] |
| 199 | 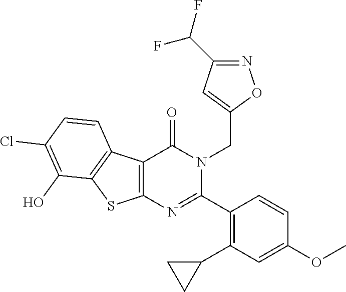 [SMILES: COc1ccc(-c2nc3sc4c(O)c(Cl)ccc4c3c(=O)n2Cc2cc(C(F)F)no2)c(C2CC2)c1] |
| 203 | 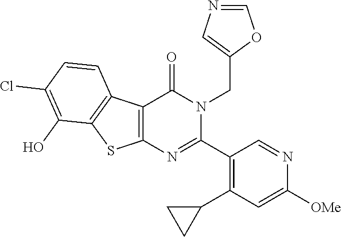 [SMILES: COc1cc(C2CC2)c(-c2nc3sc4c(O)c(Cl)ccc4c3c(=O)n2Cc2cnco2)cn1] |
| 204 | 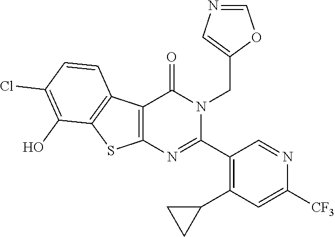 [SMILES: O=c1c2c(nc(-c3cnc(C(F)(F)F)cc3C3CC3)n1Cc1cnco1)sc1c(O)c(Cl)ccc12] |
| 206 | 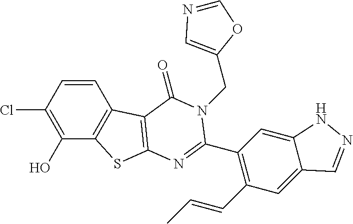 [SMILES: C/C=C/c1cc2cn[nH]c2cc1-c1nc2sc3c(O)c(Cl)ccc3c2c(=O)n1Cc1cnco1] |

### Synthesis of 7-chloro-2-(2-cyclopropyl-4-methoxyphenyl)-8-hydroxy-3-((5-methyloxazol-2-yl)methyl)benzo[4,5]thieno[2,3-d]pyrimidin-4(3H)-one (Example 200)

### Example 200

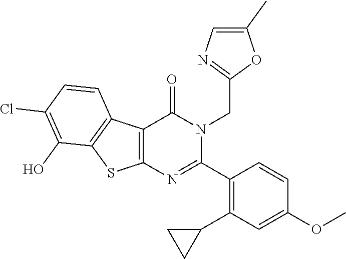 [SMILES: COc1ccc(-c2nc3sc4c(O)c(Cl)ccc4c3c(=O)n2Cc2ncc(C)o2)c(C2CC2)c1]

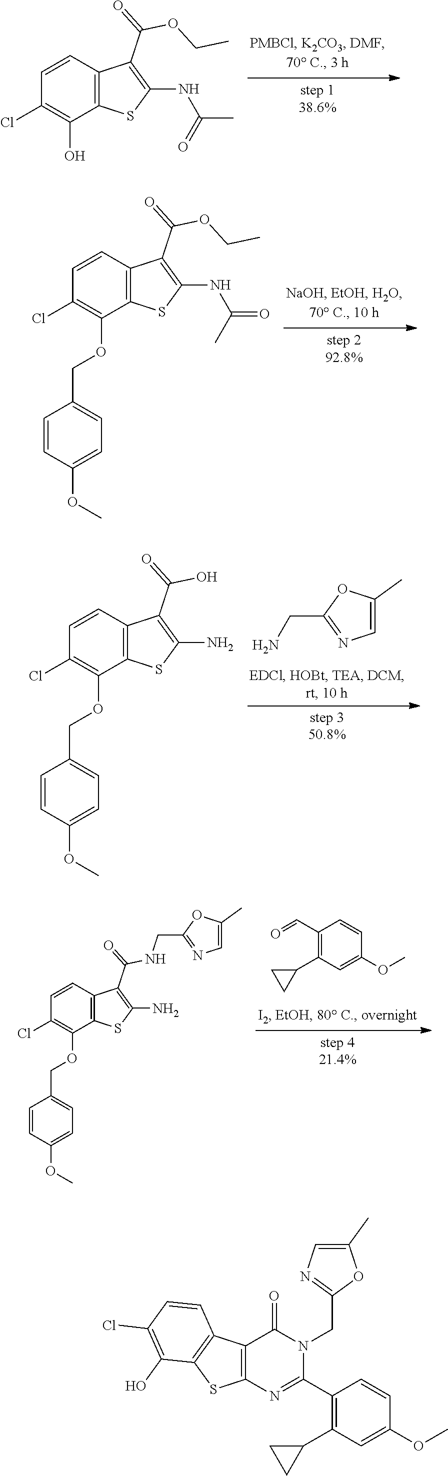

To a solution of PMBCl (1.3 g, 8.6 mmol, 0.9 eq) and ethyl 2-acetamido-6-chloro-7-hydroxybenzo[b]thiophene-3-carboxylate (3.0 g, 9.6 mmol, 1.0 eq) in DMF (50.0 mL) was added K₂CO₃ (2.6 g, 19.1 mmol, 2.0 eq), the reaction mixture was stirred at 70° C. for 3 h. The mixture was diluted with EtOAc (100 mL), washed with brine (100 mL×3). The organic phase was concentrated and purified by silica gel column chromatograph (Petroleum ether:EtOAc=5:1) to give ethyl 2-acetamido-6-chloro-7-((4-methoxybenzyl)oxy)benzo[b]thiophene-3-carboxylate (1.6 g, 38.6%) as a red solid. ¹H NMR (400 MHz, DMSO-d₆): δ 11.34 (s, 1H), 7.97 (d, J=8.8 Hz, 1H), 7.53 (d, J=8.4 Hz, 1H), 7.42 (d, J=8.4 Hz, 2H), 6.96 (d, J=8.4 Hz, 2H), 5.13 (s, 2H), 4.52-4.36 (m, 2H), 3.76 (s, 3H), 2.33 (s, 3H), 1.42 (t, J=7.2 Hz, 3H).

To a solution of ethyl 2-acetamido-6-chloro-7-((4-methoxybenzyl)oxy)benzo[b]thiophene-3-carboxylate (1.8 g, 4.1 mmol, 1.0 eq) in EtOH (15.0 mL)/THF (10.0 mL)/Water (15.0 mL) was added NaOH (996 mg, 24.9 mmol, 6.0 eq), the reaction mixture was stirred at 70° C. for 10 h. The solvent was removed, the reaction mixture was adjusted pH to 3-4 with 1 N HCl. The mixture was extracted with EtOAc (100 mL). The organic phase was dried with Na₂SO₄ and concentrated under reduce pressure to give 2-amino-6-chloro-7-((4-methoxybenzyl)oxy)benzo[b]thiophene-3-carboxylic acid (1.4 g, 92.8%) as a yellow solid. ¹H NMR (400 MHz, DMSO-d₆): δ 12.43 (s, 1H), 8.00 (s, 2H), 7.71 (d, J=8.8 Hz, 1H), 7.40 (d, J=8.4 Hz, 2H), 7.32 (d, J=8.4 Hz, 1H), 6.96 (d, J=8.8 Hz, 2H), 5.03 (s, 2H), 3.77 (s, 3H).

To a solution of (5-methyloxazol-2-yl)methanamine (58.6 mg, 0.52 mmol, 1.9 eq) and 2-amino-6-chloro-7-((4-methoxybenzyl)oxy)benzo[b]thiophene-3-carboxylic acid (100 mg, 0.275 mmol, 1.0 eq) in DCM (10.0 mL) was added EDCI·HCl (105.4 mg, 0.55 mmol, 2.0 eq), HOBT (44.6 mg, 0.33 mmol, 1.2 eq) and TEA (138.9 mg, 1.4 mmol, 5.0 eq), the reaction mixture was stirred at room temperature for 10 h. The mixture was diluted with DCM (50 mL), washed with water (20 mL). The organic phase was concentrated and purified by silica gel column chromatography (Petroleum ether:EtOAc=5:1) to give 2-amino-6-chloro-7-((4-methoxybenzyl)oxy)-N-((5-methyloxazol-2-yl)methyl)benzo[b]thiophene-3-carboxamide (64 mg, 50.8%) as a white solid. ¹H NMR (400 MHz, DMSO-d₆): δ 8.01 (t, J=5.6 Hz, 1H), 7.66 (s, 2H), 7.49 (d, J=8.4 Hz, 1H), 7.41 (d, J=8.4 Hz, 2H), 7.34 (d, J=8.8 Hz, 1H), 6.96 (d, J=8.8 Hz, 2H), 6.75 (d, J=0.8 Hz, 1H), 5.05 (s, 2H), 4.50 (d, J=5.6 Hz, 2H), 3.77 (s, 3H), 2.27 (s, 3H).

To a solution of 2-amino-6-chloro-7-((4-methoxybenzyl)oxy)-N-((5-methyloxazol-2-yl)methyl)benzo[b]thiophene-3-carboxamide (64 mg, 0.14 mmol, 1.0 eq) and 2-cyclopropyl-4-methoxybenzaldehyde (32 mg, 0.18 mmol, 1.3 eq) in EtOH (4.0 mL) was added 12 (39 mg, 0.15 mmol, 1.1 eq), the reaction mixture was stirred at 80° C. overnight. To the mixture was added 10% aq. Na₂S₂O₃ (3 mL). Concentrated and extracted with EtOAc (50 mL). The organic phase was concentrated and purified by silica gel column chromatography (Petroleum ether:EtOAc=3:1) to give 7-chloro-2-(2-cyclopropyl-4-methoxyphenyl)-8-hydroxy-3-((5-methyloxazol-2-yl)methyl)benzo[4,5]thieno[2,3-d]pyrimidin-4(3H)-one (15 mg, 21.4%) as a white solid. LC-MS (M+H)+: m/z=493.8. ¹H NMR (300 MHz, DMSO-d₆): δ 10.82 (s, 1H), 7.97 (d, J=8.4 Hz, 1H), 7.58 (d, J=8.4 Hz, 1H), 7.24 (d, J=8.7 Hz, 1H), 6.87-6.76 (m, 1H), 6.69 (d, J=1.5 Hz, 1H), 6.47 (d, J=2.4 Hz, 1H), 5.44 (d, J=16.5 Hz, 1H), 4.94 (d, J=16.5 Hz, 1H), 3.78 (s, 3H), 2.19 (d, J=1.2 Hz, 3H), 1.76-1.62 (m, 1H), 0.88-0.78 (m, 3H), 0.71-0.63 (in, 1H).

The following examples 197, 201˜202, 205, and 207˜217, were prepared according to procedures similar to example 200.

| Example # | Structure |
|---|---|
| 197 | 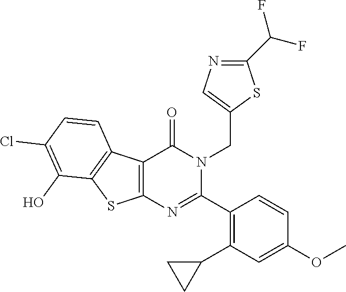 [SMILES: COc1ccc(-c2nc3sc4c(O)c(Cl)ccc4c3c(=O)n2Cc2cnc(C(F)F)s2)c(C2CC2)c1] |
| 201 | 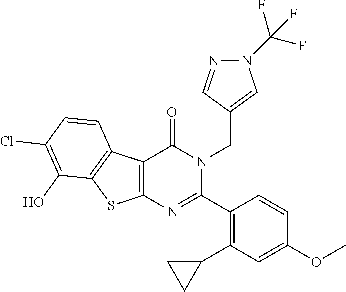 [SMILES: COc1ccc(-c2nc3sc4c(O)c(Cl)ccc4c3c(=O)n2Cc2cnn(C(F)(F)F)c2)c(C2CC2)c1] |
| 202 | 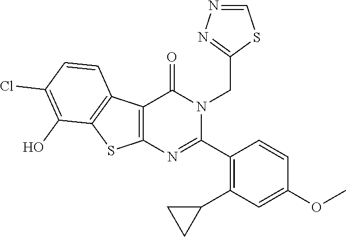 [SMILES: COc1ccc(-c2nc3sc4c(O)c(Cl)ccc4c3c(=O)n2Cc2nncs2)c(C2CC2)c1] |
| 205 | 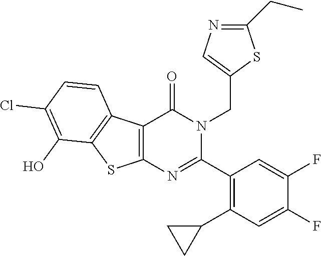 [SMILES: CCc1ncc(Cn2c(-c3cc(F)c(F)cc3C3CC3)nc3sc4c(O)c(Cl)ccc4c3c2=O)s1] |
| 207 | 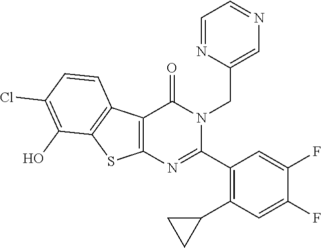 [SMILES: O=c1c2c(nc(-c3cc(F)c(F)cc3C3CC3)n1Cc1cnccn1)sc1c(O)c(Cl)ccc12] |
| 208 | 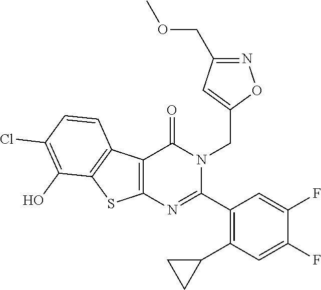 [SMILES: COCc1cc(Cn2c(-c3cc(F)c(F)cc3C3CC3)nc3sc4c(O)c(Cl)ccc4c3c2=O)on1] |
| 209 | 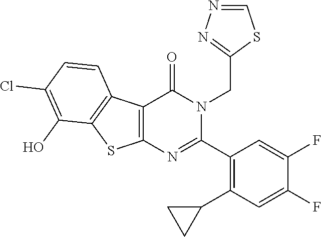 [SMILES: O=c1c2c(nc(-c3cc(F)c(F)cc3C3CC3)n1Cc1nncs1)sc1c(O)c(Cl)ccc12] |
| 210 | 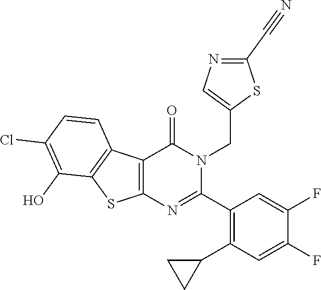 [SMILES: N#Cc1ncc(Cn2c(-c3cc(F)c(F)cc3C3CC3)nc3sc4c(O)c(Cl)ccc4c3c2=O)s1] |
| 211 | 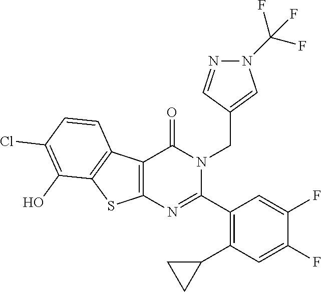 [SMILES: O=c1c2c(nc(-c3cc(F)c(F)cc3C3CC3)n1Cc1cnn(C(F)(F)F)c1)sc1c(O)c(Cl)ccc12] |
| 212 | 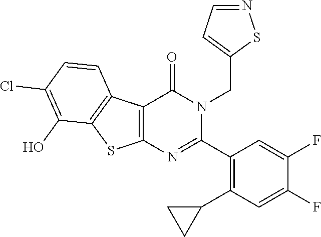 [SMILES: O=c1c2c(nc(-c3cc(F)c(F)cc3C3CC3)n1Cc1ccns1)sc1c(O)c(Cl)ccc12] |
| 213 | 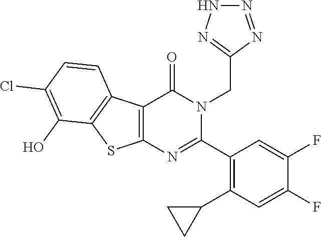 [SMILES: O=c1c2c(nc(-c3cc(F)c(F)cc3C3CC3)n1Cc1nn[nH]n1)sc1c(O)c(Cl)ccc12] |
| 214 | 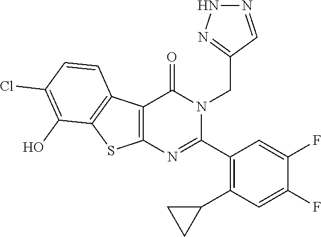 [SMILES: O=c1c2c(nc(-c3cc(F)c(F)cc3C3CC3)n1Cc1cn[nH]n1)sc1c(O)c(Cl)ccc12] |
| 215 | 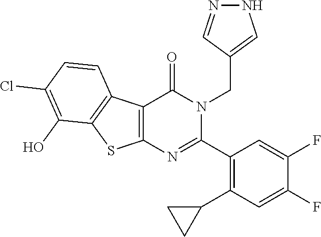 [SMILES: O=c1c2c(nc(-c3cc(F)c(F)cc3C3CC3)n1Cc1cn[nH]c1)sc1c(O)c(Cl)ccc12] |
| 216 | 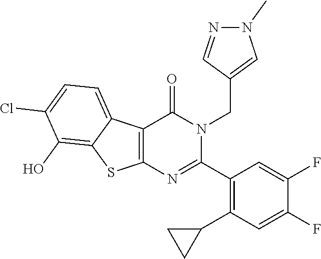 [SMILES: Cn1cc(Cn2c(-c3cc(F)c(F)cc3C3CC3)nc3sc4c(O)c(Cl)ccc4c3c2=O)cn1] |
| 217 | 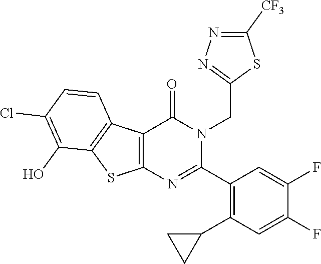 [SMILES: O=c1c2c(nc(-c3cc(F)c(F)cc3C3CC3)n1Cc1nnc(C(F)(F)F)s1)sc1c(O)c(Cl)ccc12] |

### Synthesis of 7-chloro-2-(2-cyclopropyl-4-methoxyphenyl)-5-fluoro-8-hydroxy-3-(oxazol-5-ylmethyl)benzo[4,5]thieno[2,3-d]pyrimidin-4(3H)-one (Example 218)

### Example 218

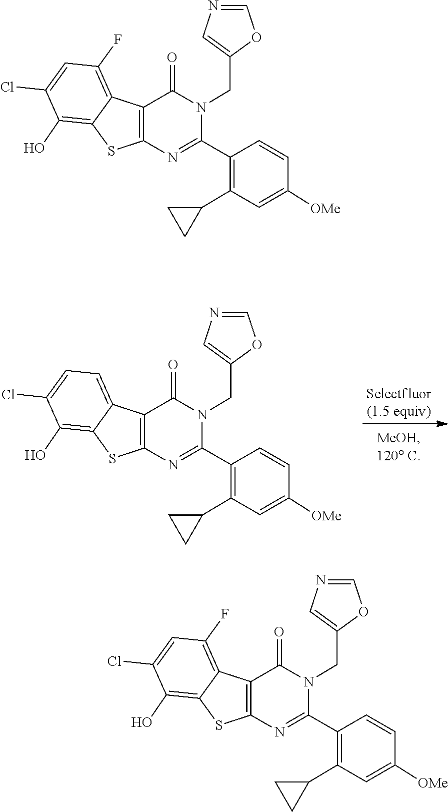 [SMILES: COc1ccc(-c2nc3sc4c(O)c(Cl)cc(F)c4c3c(=O)n2Cc2cnco2)c(C2CC2)c1 ; COc1ccc(-c2nc3sc4c(O)c(Cl)ccc4c3c(=O)n2Cc2cnco2)c(C2CC2)c1 ; COc1ccc(-c2nc3sc4c(O)c(Cl)cc(F)c4c3c(=O)n2Cc2cnco2)c(C2CC2)c1]

To a microwave reactor was charged compound 7-chloro-2-(2-cyclopropyl-4-methoxyphenyl)-8-hydroxy-3-(oxazol-5-ylmethyl)benzo[4,5]thieno[2,3-d]pyrimidin-4(3H)-one (20 mg, 0.042 mmol), selectfluor (22 mg, 0.062 mmol), and MeOH (0.5 mL). The mixture was heated to 120° C. under radiation for 10 mins. Upon completion, the mixture was evaporated to dryness. The residue was purified by preparative HPLC to afford compound 7-chloro-2-(2-cyclopropyl-4-methoxyphenyl)-5-fluoro-8-hydroxy-3-(oxazol-5-ylmethyl)benzo[4,5]thieno[2,3-d]pyrimidin-4(3H)-one as a white solid 2.5 mg, 12% yield. LC-MS observed [M+H], 498.1.

### Synthesis of 7-chloro-2-(2-cyclopropyl-4,5-difluorophenyl)-5-fluoro-8-hydroxy-3-((3-(trifluoromethyl)isoxazol-5-yl)methyl)benzo[4,5]thieno[2,3-d]pyrimidin-4(3H)-one (Example 219)

### Example 219

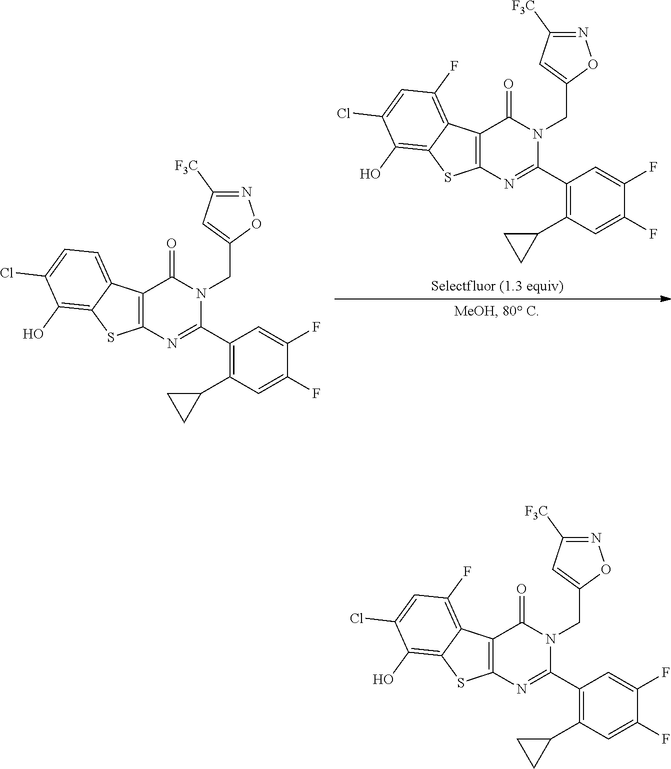 [SMILES: O=c1c2c(nc(-c3cc(F)c(F)cc3C3CC3)n1Cc1cc(C(F)(F)F)no1)sc1c(O)c(Cl)ccc12 ; O=c1c2c(nc(-c3cc(F)c(F)cc3C3CC3)n1Cc1cc(C(F)(F)F)no1)sc1c(O)c(Cl)cc(F)c12 ; O=c1c2c(nc(-c3cc(F)c(F)cc3C3CC3)n1Cc1cc(C(F)(F)F)no1)sc1c(O)c(Cl)cc(F)c12]

To a microwave reactor was charged compound 7-chloro-2-(2-cyclopropyl-4,5-difluorophenyl)-8-hydroxy-3-((3-(trifluoromethyl)isoxazol-5-yl)methyl)benzo[4,5]thieno[2,3-d]pyrimidin-4(3H)-one (32 mg, 0.058 mmol), selectfluor (27 mg, 0.076 mmol), and MeOH (1.5 mL). The mixture was heated to 80° C. under radiation for 10 mins. Upon completion, the mixture was evaporated to dryness. The residue was purified by preparative HPLC to afford compound 7-chloro-2-(2-cyclopropyl-4,5-difluorophenyl)-5-fluoro-8-hydroxy-3-((3-(trifluoromethyl)isoxazol-5-yl)methyl)benzo[4,5]thieno[2,3-d]pyrimidin-4(3H)-one as a white solid 1.2 mg, 4% yield. LC-MS observed [M+H], 571.8.

The following examples 220˜222, were prepared according to procedures described previously.

| Example # | Structure |
|---|---|
| 220 | 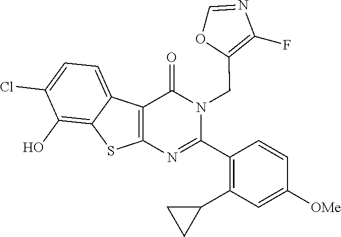 [SMILES: COc1ccc(-c2nc3sc4c(O)c(Cl)ccc4c3c(=O)n2Cc2ocnc2F)c(C2CC2)c1] |
| 221 | 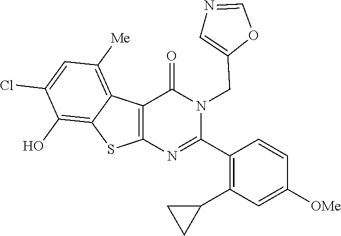 [SMILES: COc1ccc(-c2nc3sc4c(O)c(Cl)cc(C)c4c3c(=O)n2Cc2cnco2)c(C2CC2)c1] |
| 222 | 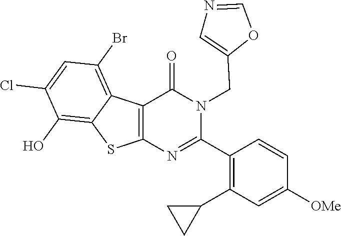 [SMILES: COc1ccc(-c2nc3sc4c(O)c(Cl)cc(Br)c4c3c(=O)n2Cc2cnco2)c(C2CC2)c1] |

### Synthesis of 2-(2-cyclopropyl-4-methoxyphenyl)-5,7-difluoro-8-hydroxy-3-(oxazol-5-ylmethyl)benzo[4,5]thieno[2,3-d]pyrimidin-4(3H)-one (Example 223)

### Example 223

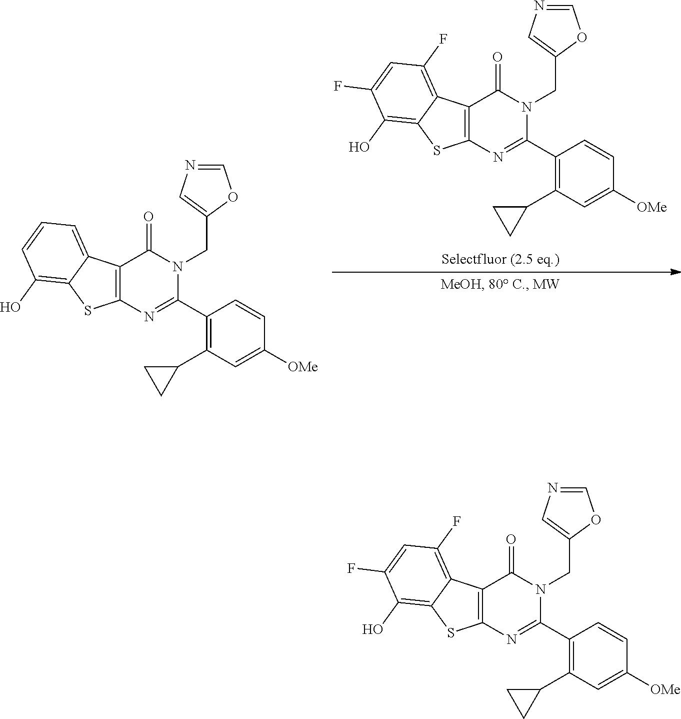 [SMILES: COc1ccc(-c2nc3sc4c(O)cccc4c3c(=O)n2Cc2cnco2)c(C2CC2)c1 ; COc1ccc(-c2nc3sc4c(O)c(F)cc(F)c4c3c(=O)n2Cc2cnco2)c(C2CC2)c1 ; COc1ccc(-c2nc3sc4c(O)c(F)cc(F)c4c3c(=O)n2Cc2cnco2)c(C2CC2)c1]

To a microwave reactor was charged 2-(2-cyclopropyl-4-methoxyphenyl)-8-hydroxy-3-(oxazol-5-ylmethyl)benzo[4,5]thieno[2,3-d]pyrimidin-4(3H)-one (20.0 mg, 1 Eq, 44.9 μmol) and selectfluor (39.8 mg, 2.5 Eq, 112 μmol). The reactor was purged with N₂, followed by the addition of methanol (0.5 mL). The reaction was heated to 80° C. under radiation for 30 mins. Upon completion, the solvent was removed, and the desired product was purified through normal phase column chromatography followed by the pre-HPLC separation. LC-MS observed [M+H], 482.1.

The following examples in Table 1 are prepared by using procedures similar to those described above.

TABLE 1

| Example # | Structure |
|---|---|
| P1 | 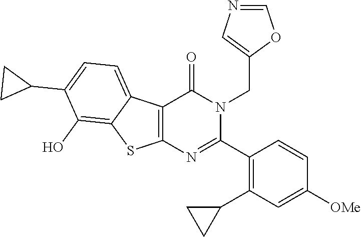 [SMILES: COc1ccc(-c2nc3sc4c(O)c(C5CC5)ccc4c3c(=O)n2Cc2cnco2)c(C2CC2)c1] |
| P2 | 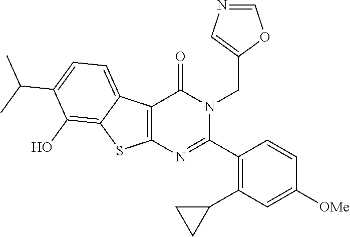 [SMILES: COc1ccc(-c2nc3sc4c(O)c(C(C)C)ccc4c3c(=O)n2Cc2cnco2)c(C2CC2)c1] |
| P3 | 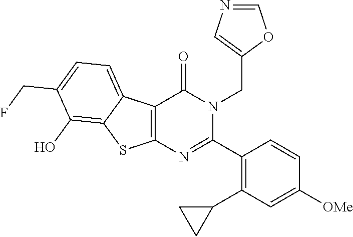 [SMILES: COc1ccc(-c2nc3sc4c(O)c(CF)ccc4c3c(=O)n2Cc2cnco2)c(C2CC2)c1] |
| P4 | 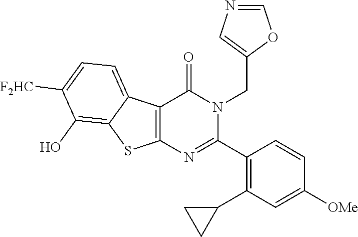 [SMILES: COc1ccc(-c2nc3sc4c(O)c(C(F)F)ccc4c3c(=O)n2Cc2cnco2)c(C2CC2)c1] |
| P5 | 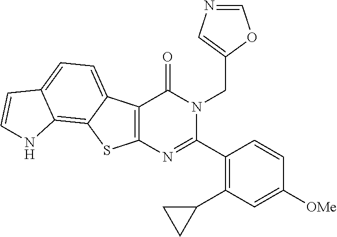 [SMILES: COc1ccc(-c2nc3sc4c(ccc5cc[nH]c54)c3c(=O)n2Cc2cnco2)c(C2CC2)c1] |
| P6 | 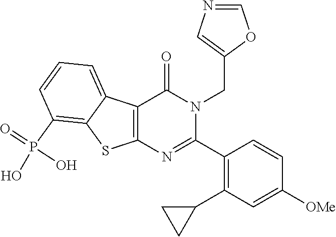 [SMILES: COc1ccc(-c2nc3sc4c(P(=O)(O)O)cccc4c3c(=O)n2Cc2cnco2)c(C2CC2)c1] |
| P7 | 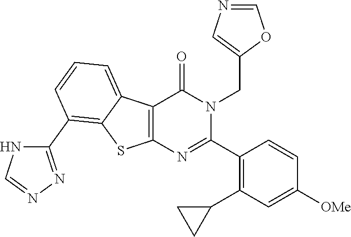 [SMILES: COc1ccc(-c2nc3sc4c(-c5nnc[nH]5)cccc4c3c(=O)n2Cc2cnco2)c(C2CC2)c1] |
| P8 | 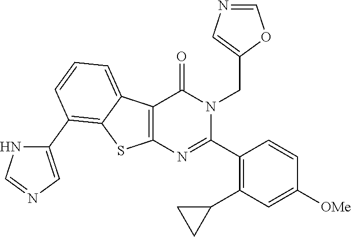 [SMILES: COc1ccc(-c2nc3sc4c(-c5cnc[nH]5)cccc4c3c(=O)n2Cc2cnco2)c(C2CC2)c1] |
| P9 | 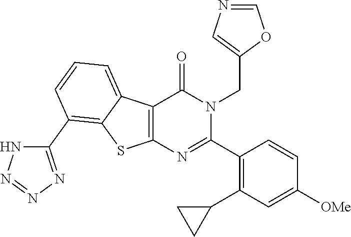 [SMILES: COc1ccc(-c2nc3sc4c(-c5nnn[nH]5)cccc4c3c(=O)n2Cc2cnco2)c(C2CC2)c1] |
| P10 | 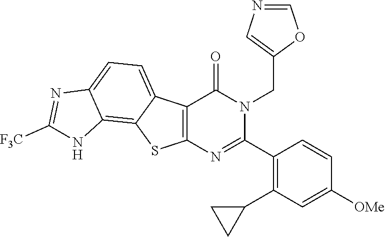 [SMILES: COc1ccc(-c2nc3sc4c(ccc5nc(C(F)(F)F)[nH]c54)c3c(=O)n2Cc2cnco2)c(C2CC2)c1] |
| P11 | 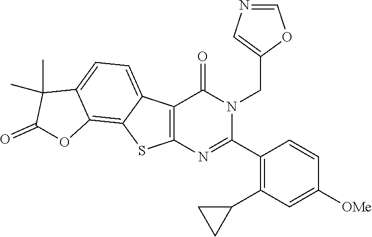 [SMILES: COc1ccc(-c2nc3sc4c5c(ccc4c3c(=O)n2Cc2cnco2)C(C)(C)C(=O)O5)c(C2CC2)c1] |
| P12 | 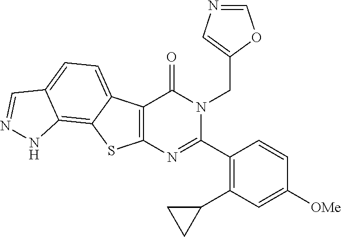 [SMILES: COc1ccc(-c2nc3sc4c(ccc5cn[nH]c54)c3c(=O)n2Cc2cnco2)c(C2CC2)c1] |
| P13 | 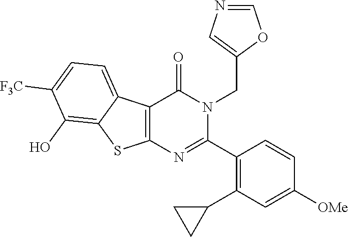 [SMILES: COc1ccc(-c2nc3sc4c(O)c(C(F)(F)F)ccc4c3c(=O)n2Cc2cnco2)c(C2CC2)c1] |
| P14 | 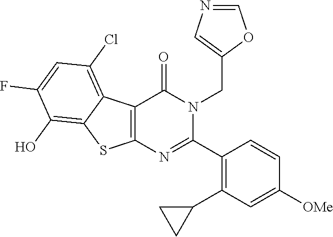 [SMILES: COc1ccc(-c2nc3sc4c(O)c(F)cc(Cl)c4c3c(=O)n2Cc2cnco2)c(C2CC2)c1] |
| P15 | 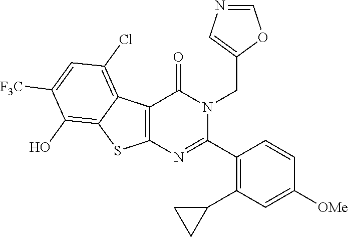 [SMILES: COc1ccc(-c2nc3sc4c(O)c(C(F)(F)F)cc(Cl)c4c3c(=O)n2Cc2cnco2)c(C2CC2)c1] |
| P16 | 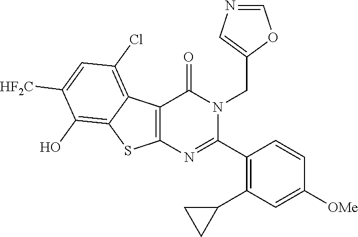 |
| P17 | 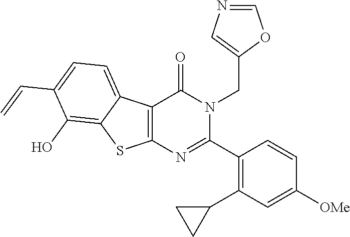 [SMILES: C=Cc1ccc2c(sc3nc(-c4ccc(OC)cc4C4CC4)n(Cc4cnco4)c(=O)c32)c1O] |
| P18 | 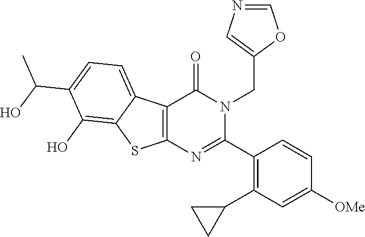 [SMILES: COc1ccc(-c2nc3sc4c(O)c(C(C)O)ccc4c3c(=O)n2Cc2cnco2)c(C2CC2)c1] |
| P19 | 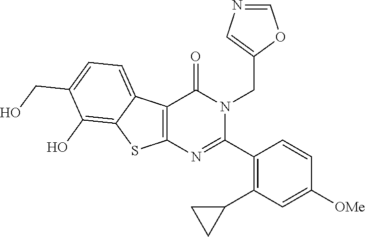 [SMILES: COc1ccc(-c2nc3sc4c(O)c(CO)ccc4c3c(=O)n2Cc2cnco2)c(C2CC2)c1] |
| P20 | 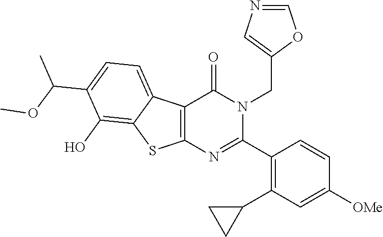 [SMILES: COc1ccc(-c2nc3sc4c(O)c(C(C)OC)ccc4c3c(=O)n2Cc2cnco2)c(C2CC2)c1] |
| P21 | 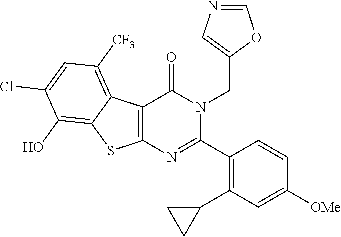 [SMILES: COc1ccc(-c2nc3sc4c(O)c(Cl)cc(C(F)(F)F)c4c3c(=O)n2Cc2cnco2)c(C2CC2)c1] |
| P22 | 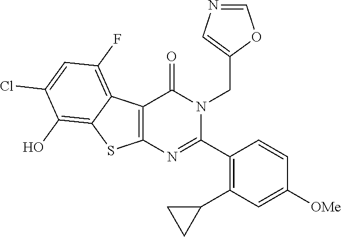 [SMILES: COc1ccc(-c2nc3sc4c(O)c(Cl)cc(F)c4c3c(=O)n2Cc2cnco2)c(C2CC2)c1] |
| P23 | 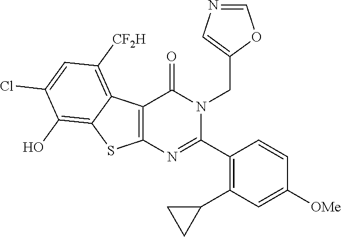 [SMILES: COc1ccc(-c2nc3sc4c(O)c(Cl)cc(C(F)F)c4c3c(=O)n2Cc2cnco2)c(C2CC2)c1] |
| P24 | 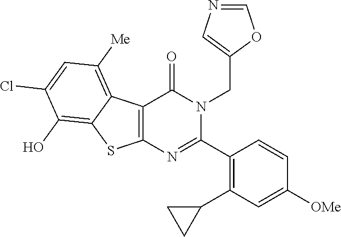 [SMILES: COc1ccc(-c2nc3sc4c(O)c(Cl)cc(C)c4c3c(=O)n2Cc2cnco2)c(C2CC2)c1] |
| P25 | 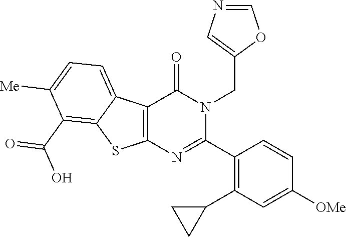 [SMILES: COc1ccc(-c2nc3sc4c(C(=O)O)c(C)ccc4c3c(=O)n2Cc2cnco2)c(C2CC2)c1] |
| P26 | 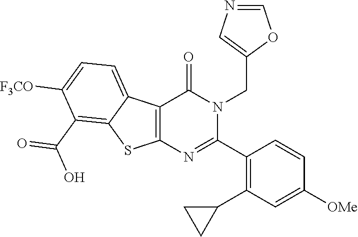 [SMILES: COc1ccc(-c2nc3sc4c(C(=O)O)c(OC(F)(F)F)ccc4c3c(=O)n2Cc2cnco2)c(C2CC2)c1] |
| P27 | 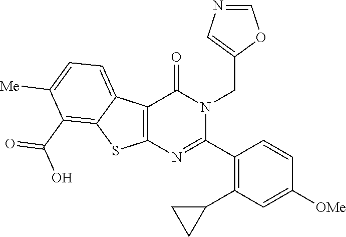 [SMILES: COc1ccc(-c2nc3sc4c(C(=O)O)c(C)ccc4c3c(=O)n2Cc2cnco2)c(C2CC2)c1] |
| P28 | 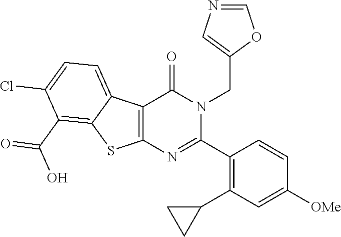 [SMILES: COc1ccc(-c2nc3sc4c(C(=O)O)c(Cl)ccc4c3c(=O)n2Cc2cnco2)c(C2CC2)c1] |
| P29 | 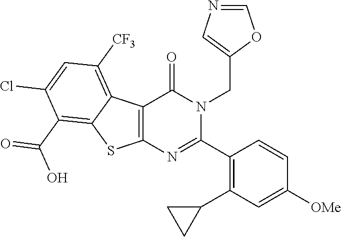 [SMILES: COc1ccc(-c2nc3sc4c(C(=O)O)c(Cl)cc(C(F)(F)F)c4c3c(=O)n2Cc2cnco2)c(C2CC2)c1] |
| P30 | 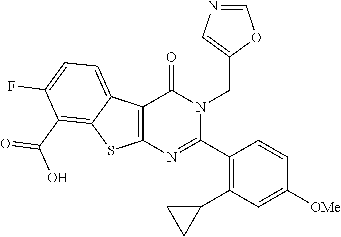 [SMILES: COc1ccc(-c2nc3sc4c(C(=O)O)c(F)ccc4c3c(=O)n2Cc2cnco2)c(C2CC2)c1] |
| P31 | 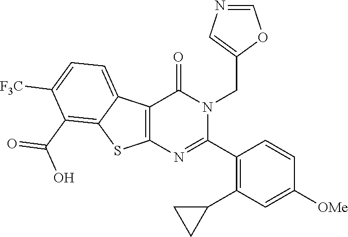 [SMILES: COc1ccc(-c2nc3sc4c(C(=O)O)c(C(F)(F)F)ccc4c3c(=O)n2Cc2cnco2)c(C2CC2)c1] |
| P32 | 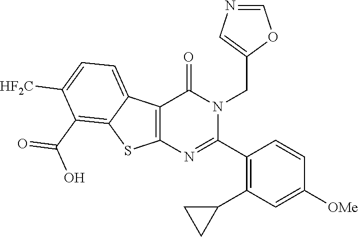 |
| P33 | 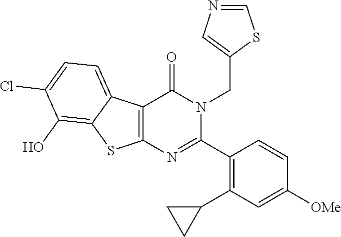 [SMILES: COc1ccc(-c2nc3sc4c(O)c(Cl)ccc4c3c(=O)n2Cc2cncs2)c(C2CC2)c1] |
| P34 | 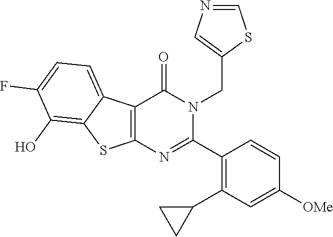 [SMILES: COc1ccc(-c2nc3sc4c(O)c(F)ccc4c3c(=O)n2Cc2cncs2)c(C2CC2)c1] |
| P35 | 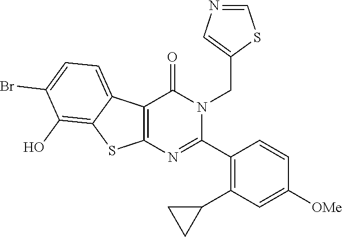 [SMILES: COc1ccc(-c2nc3sc4c(O)c(Br)ccc4c3c(=O)n2Cc2cncs2)c(C2CC2)c1] |
| P36 | 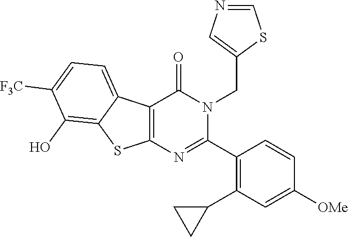 [SMILES: COc1ccc(-c2nc3sc4c(O)c(C(F)(F)F)ccc4c3c(=O)n2Cc2cncs2)c(C2CC2)c1] |
| P37 | 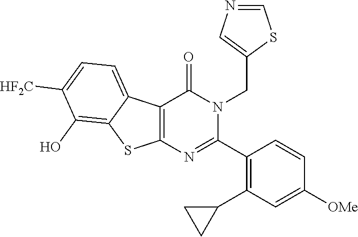 |
| P38 | 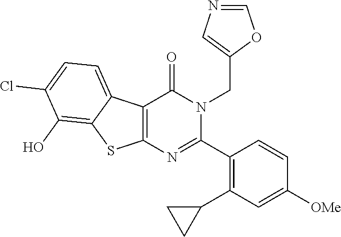 [SMILES: COc1ccc(-c2nc3sc4c(O)c(Cl)ccc4c3c(=O)n2Cc2cnco2)c(C2CC2)c1] |
| P39 | 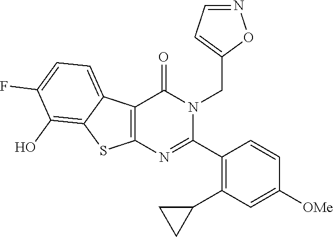 [SMILES: COc1ccc(-c2nc3sc4c(O)c(F)ccc4c3c(=O)n2Cc2ccno2)c(C2CC2)c1] |
| P40 | 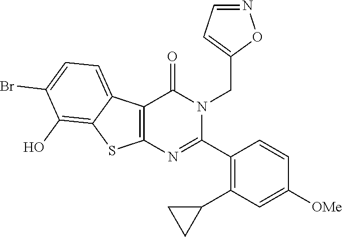 [SMILES: COc1ccc(-c2nc3sc4c(O)c(Br)ccc4c3c(=O)n2Cc2ccno2)c(C2CC2)c1] |
| P41 | 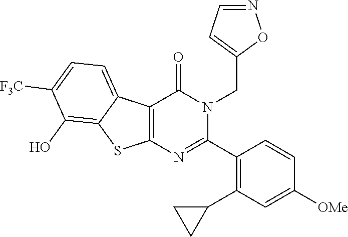 [SMILES: COc1ccc(-c2nc3sc4c(O)c(C(F)(F)F)ccc4c3c(=O)n2Cc2ccno2)c(C2CC2)c1] |
| P42 | 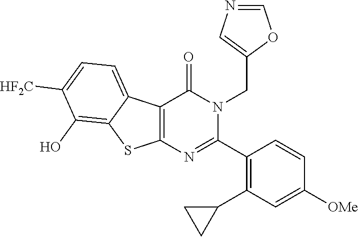 |
| P43 | 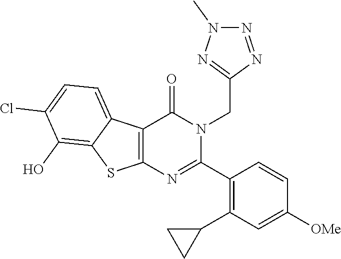 [SMILES: COc1ccc(-c2nc3sc4c(O)c(Cl)ccc4c3c(=O)n2Cc2nnn(C)n2)c(C2CC2)c1] |
| P44 | 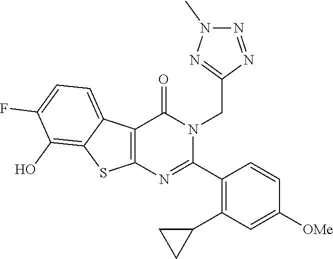 [SMILES: COc1ccc(-c2nc3sc4c(O)c(F)ccc4c3c(=O)n2Cc2nnn(C)n2)c(C2CC2)c1] |
| P45 | 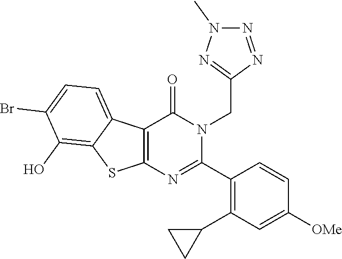 [SMILES: COc1ccc(-c2nc3sc4c(O)c(Br)ccc4c3c(=O)n2Cc2nnn(C)n2)c(C2CC2)c1] |
| P46 | 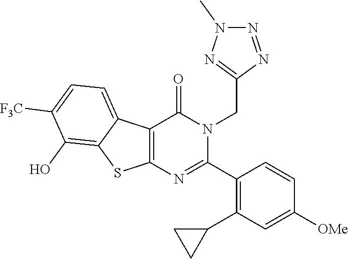 [SMILES: COc1ccc(-c2nc3sc4c(O)c(C(F)(F)F)ccc4c3c(=O)n2Cc2nnn(C)n2)c(C2CC2)c1] |
| P47 | 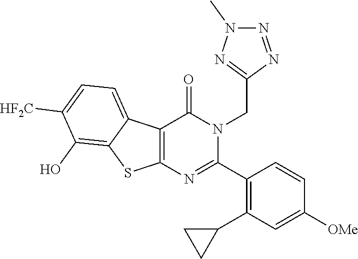 |
| P48 | 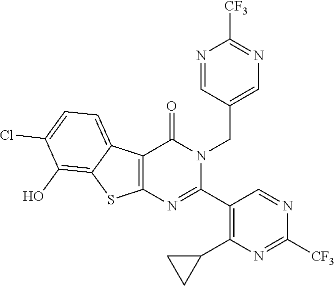 [SMILES: O=c1c2c(nc(-c3cnc(C(F)(F)F)nc3C3CC3)n1Cc1cnc(C(F)(F)F)nc1)sc1c(O)c(Cl)ccc12] |
| P49 | 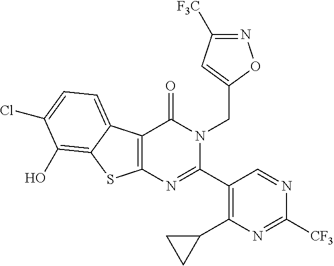 [SMILES: O=c1c2c(nc(-c3cnc(C(F)(F)F)nc3C3CC3)n1Cc1cc(C(F)(F)F)no1)sc1c(O)c(Cl)ccc12] |
| P50 | 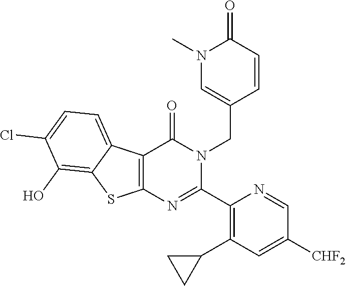 [SMILES: Cn1cc(Cn2c(-c3ncc(C(F)F)cc3C3CC3)nc3sc4c(O)c(Cl)ccc4c3c2=O)ccc1=O] |
| P51 | 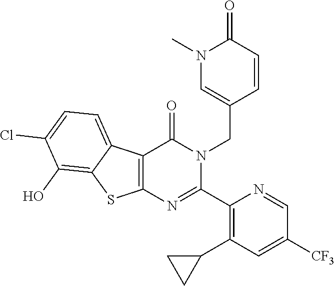 [SMILES: Cn1cc(Cn2c(-c3ncc(C(F)(F)F)cc3C3CC3)nc3sc4c(O)c(Cl)ccc4c3c2=O)ccc1=O] |
| P52 | 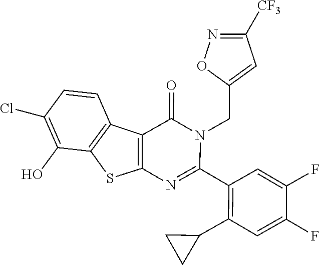 [SMILES: O=c1c2c(nc(-c3cc(F)c(F)cc3C3CC3)n1Cc1cc(C(F)(F)F)no1)sc1c(O)c(Cl)ccc12] |
| P53 | 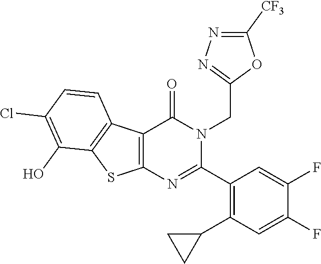 [SMILES: O=c1c2c(nc(-c3cc(F)c(F)cc3C3CC3)n1Cc1nnc(C(F)(F)F)o1)sc1c(O)c(Cl)ccc12] |
| P54 | 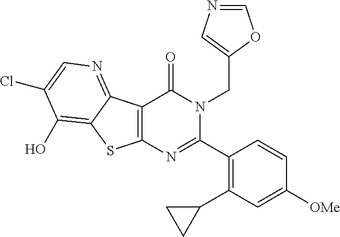 [SMILES: COc1ccc(-c2nc3sc4c(O)c(Cl)cnc4c3c(=O)n2Cc2cnco2)c(C2CC2)c1] |
| P55 | 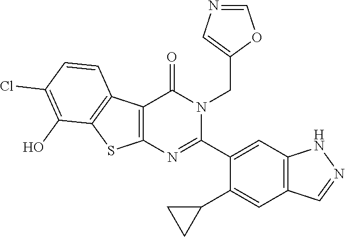 [SMILES: O=c1c2c(nc(-c3cc4[nH]ncc4cc3C3CC3)n1Cc1cnco1)sc1c(O)c(Cl)ccc12] |
| P56 | 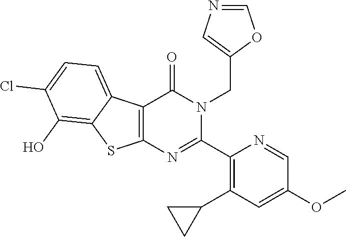 [SMILES: COc1cnc(-c2nc3sc4c(O)c(Cl)ccc4c3c(=O)n2Cc2cnco2)c(C2CC2)c1] |
| P57 | 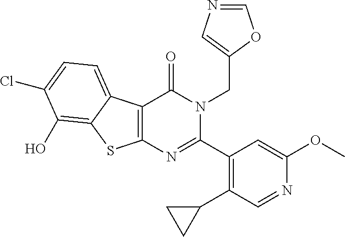 [SMILES: COc1cc(-c2nc3sc4c(O)c(Cl)ccc4c3c(=O)n2Cc2cnco2)c(C2CC2)cn1] |
| P58 | 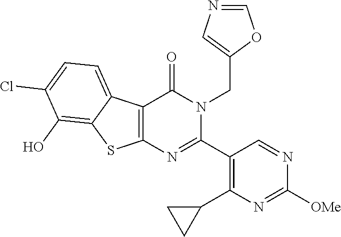 [SMILES: COc1ncc(-c2nc3sc4c(O)c(Cl)ccc4c3c(=O)n2Cc2cnco2)c(C2CC2)n1] |
| P59 | 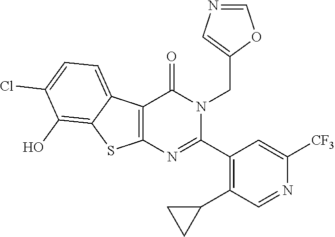 [SMILES: O=c1c2c(nc(-c3cc(C(F)(F)F)ncc3C3CC3)n1Cc1cnco1)sc1c(O)c(Cl)ccc12] |
| P60 | 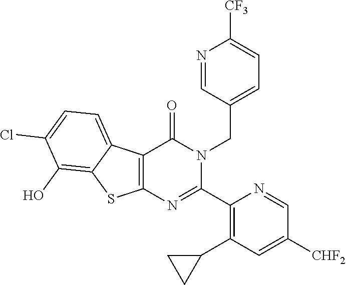 [SMILES: O=c1c2c(nc(-c3ncc(C(F)F)cc3C3CC3)n1Cc1ccc(C(F)(F)F)nc1)sc1c(O)c(Cl)ccc12] |
| P61 | 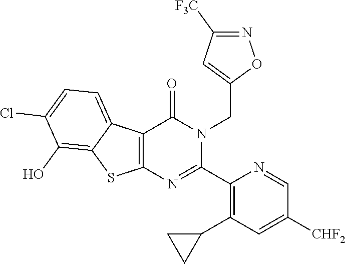 [SMILES: O=c1c2c(nc(-c3ncc(C(F)F)cc3C3CC3)n1Cc1cc(C(F)(F)F)no1)sc1c(O)c(Cl)ccc12] |
| P62 | 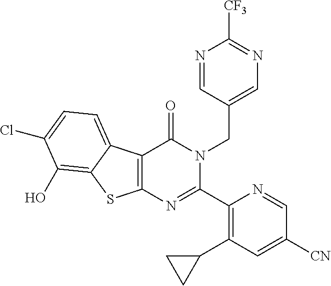 [SMILES: N#Cc1cnc(-c2nc3sc4c(O)c(Cl)ccc4c3c(=O)n2Cc2cnc(C(F)(F)F)nc2)c(C2CC2)c1] |
| P63 | 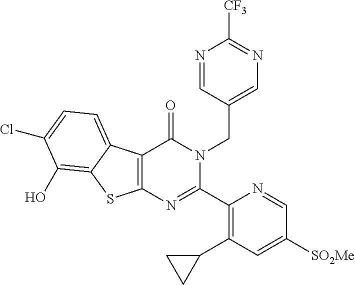 [SMILES: CS(=O)(=O)c1cnc(-c2nc3sc4c(O)c(Cl)ccc4c3c(=O)n2Cc2cnc(C(F)(F)F)nc2)c(C2CC2)c1] |
| P64 | 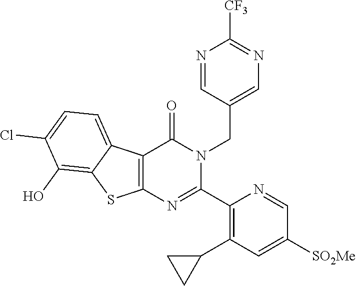 [SMILES: CS(=O)(=O)c1cnc(-c2nc3sc4c(O)c(Cl)ccc4c3c(=O)n2Cc2cnc(C(F)(F)F)nc2)c(C2CC2)c1] |
| P65 | 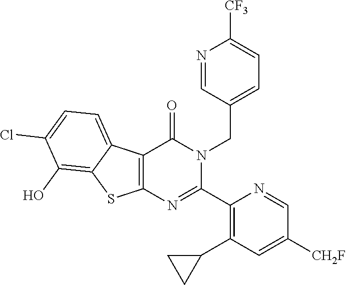 |
| P66 | 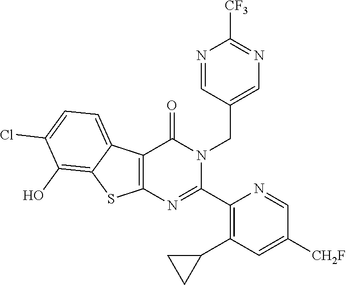 |
| P67 | 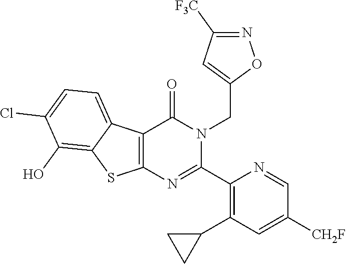 |

### Assay

17(3-HSD13 rapid-fire mass spectrometry assay (RF/MS assay). Recombinant human 17β-HSD13 was expressed and purified from sf9 cells at Charles River Labs (Saffron Walden, UK). Leukotriene B4 (Catalog #71160-24-2) and 12-oxoleukotriene B4 (Catalog #20140) were purchased from Cayman Chemicals (Ann Arbor, MI). NAD+ (Catalog #N8285), BSA (Catalog #A7030), DMSO (Catalog #D2650), and Tween-20 (Catalog #11332465001) were purchased from Sigma (St. Louis, MO). Formic acid (Catalog #28905) was from ThermoFisher Scientific and 384 deep well PP microplates (Catalog #784261) were from Greiner Bio-One. In a typical IC₅₀ assay performed in a 384w PP microplate, test compounds (0-100 μM) were incubated with HSD17B13 (80 nM), LTB4 (10 μM), and NAD⁺ (0.5 mM) in 10 μL assay buffer (20 mM Tris (pH 7.5), BSA (0.005%), and Tween-20 (0.01%)) at RT for 3 h. The assays were quenched by adding 20 μL of 0.15% aqueous formic acid and the plates were frozen at −80° C. RF/MS analysis was performed at PureHoney Technologies (Billerica, MA) on a RapidFire RF300 system (Agilent Technologies, Inc.) coupled to an API 4000 triple quadrupole mass spectrometer (Sciex) equipped with Agilent RapidFire cartridge type A (C4). The mobile phase was 0.09% formic acid and 0.01% trifluoracetic acid in water (Buffer A) and 0.09% formic acid and 0.01% trifluoracetic acid in 80% aqueous acetonitrile (Buffer B). The RapidFire method conditions were the following: 250 ms aspirate, 3000 ms load/desalt, 4000 ms elute, and 500 ms re-equilibrate. RF-MS/MS was performed in negative polarity (−4500 V), the source temperature was 650° C., and gas 1 and gas 2 settings for nitrogen were set to 50. The curtain gas and collision gas were also nitrogen and were set to 20 and 12, respectively. Leukotriene B4 (335.3) and 12-oxoLeukotriene B4 (333.3) SRM transitions were optimized with Discovery Quant software and extracted ion counts for these analytes were determined.

Data Analysis. 17β-HSD13 enzyme activity was measured as percent conversion of extracted ion counts and normalized to high and low controls to determine percent residual activity at various concentrations of test compounds. Data was fitted to normalized activity (variable slope) versus concentration fit in GraphPad Prism 7 to determine IC₅₀. All experiments were run in duplicates.

By using the above method, the inhibition of 17β-HSD13 was evaluated for the compounds of Formula (I). IC₅₀ ranges are as follows: A is <0.1 μM; B is 0.1 μM-1.0 μM; C is 1.0 μM-10 μm; and D is >10 μM.

| Example | IC₅₀ |
|---|---|
| 1 | D |
| 2 | B |
| 3 | C |
| 4 | D |
| 5 | D |
| 6 | D |
| 7 | D |
| 8 | D |
| 9 | D |
| 10 | D |
| 11 | B |
| 12 | — |
| 13 | D |
| 14 | D |
| 15 | D |
| 16 | D |
| 17 | C |
| 18 | B |
| 19 | B |
| 20 | D |
| 21 | B |
| 22 | C |
| 23 | B |
| 24 | A |
| 25 | A |
| 26 | A |
| 27 | — |
| 28 | B |
| 29 | D |
| 30 | D |
| 31 | — |
| 32 | A |
| 33 | — |
| 34 | — |
| 35 | — |
| 36 | D |
| 37 | — |
| 38 | — |
| 39 | A |
| 40 | A |
| 41 | A |
| 42 | A |
| 43 | A |
| 44 | A |
| 45 | — |
| 46 | — |
| 47 | — |
| 48 | A |
| 49 | — |
| 50 | A |
| 51 | — |
| 52 | A |
| 53 | — |
| 54 | — |
| 55 | — |
| 56 | — |
| 57 | — |
| 58 | — |
| 59 | — |
| 60 | — |
| 61 | — |
| 62 | — |
| 63 | A |
| 64 | A |
| 65 | — |
| 66 | A |
| 67 | — |
| 68 | — |
| 69 | — |
| 70 | — |
| 71 | A |
| 72 | A |
| 73 | A |
| 74 | — |
| 75 | A |
| 76 | A |
| 77 | A |
| 78 | B |
| 79 | A |
| 80 | — |
| 81 | — |
| 82 | A |
| 83 | A |
| 84 | — |
| 85 | A |
| 86 | A |
| 87 | — |
| 88 | — |
| 89 | — |
| 90 | — |
| 91 | — |
| 92 | — |
| 93 | — |
| 94 | — |
| 95 | — |
| 96 | A |
| 97 | A |
| 98 | — |
| 99 | A |
| 100 | — |
| 101 | — |
| 102 | — |
| 103 | — |
| 104 | — |
| 105 | A |
| 106 | A |
| 107 | — |
| 108 | — |
| 109 | — |
| 110 | — |
| 111 | — |
| 112 | — |
| 113 | — |
| 114 | — |
| 115 | — |
| 116 | — |
| 117 | A |
| 118 | — |
| 119 | A |
| 120 | A |
| 121 | — |
| 122 | A |
| 123 | A |
| 124 | — |
| 125 | A |
| 126 | — |
| 127 | — |
| 128 | — |
| 129 | — |
| 130 | A |
| 131 | A |
| 132 | A |
| 133 | A |
| 134 | — |
| 135 | A |
| 136 | — |
| 137 | A |
| 138 | A |
| 139 | — |
| 140 | — |
| 141 | — |
| 142 | — |
| 143 | A |
| 144 | A |
| 145 | — |
| 146 | — |
| 147 | A |
| 148 | — |
| 149 | — |
| 150 | — |
| 151 | — |
| 152 | — |
| 153 | — |
| 154 | — |
| 155 | — |
| 156 | — |
| 157 | — |
| 158 | — |
| 159 | — |
| 160 | — |
| 161 | — |
| 162 | — |
| 163 | — |
| 164 | — |
| 165 | — |
| 166 | — |
| 167 | — |
| 168 | — |
| 169 | A |
| 170 | — |
| 171 | A |
| 172 | — |
| 173 | — |
| 174 | A |
| 175 | A |
| 176 | A |
| 177 | — |
| 178 | — |
| 179 | — |
| 180 | — |
| 181 | — |
| 182 | — |
| 183 | — |
| 184 | — |
| 185 | — |
| 186 | — |
| 187 | — |
| 188 | — |
| 189 | — |
| 190 | A |
| 191 | A |
| 192 | — |
| 193 | — |
| 194 | — |
| 195 | — |
| 196 | — |
| 197 | — |
| 198 | — |
| 199 | — |
| 200 | — |
| 201 | — |
| 202 | — |
| 203 | — |
| 204 | — |
| 205 | — |
| 206 | — |
| 207 | — |
| 208 | — |
| 209 | — |
| 210 | — |
| 211 | — |
| 212 | — |
| 213 | — |
| 214 | — |
| 215 | — |
| 216 | — |
| 217 | — |
| 218 | — |
| 219 | — |
| 220 | A |
| 221 | — |
| 222 | — |
| 223 | — |

### HSD17β13 Inhibitor Cell-Based Screening Assay

The purpose of this cell-based assay is to determine the cellular efficacy (IC₅₀) of HSD17β13 inhibitors in a stable HEK293 T cell line overexpressing human HSD17β13. The assay measures by Rapid Fire mass-spectrometry (RF-MS) the conversion of estradiol-d₂ to estrone-d₂. Cytotoxicity was also assessed via luminescent signal generated by the ATPlite viability assay reagent.

### Methods:

### Overview

To screen compounds for HSD17013 inhibition, black 384-well plates were seeded with 1000× concentration of compounds in a 9-point 1:3 dilution curve (final top concentration of 10 μM) as well as 1000× concentration of 17β-estradiol-d₂ (5 μM final concentration) added to all wells. HEK293T cells stably overexpressing human HSD17013 and the parent HEK293T cell lines were then seeded in the plates on top of the compounds and substrate in MEM media containing 1% charcoal/dextran-treated FBS. Supernatant is collected and immediately frozen after 24 hours of incubation, and conversion of estradiol-d₂ to estrone-d₂ is measured at Pure Honey by RF-MS. The remaining media in the plate was removed from the wells and replaced with ATPlite viability assay reagent. Luminescent signal from the ATPlite reagent was measured on the Envision plate reader.

Seeding the Assay Plate with Compounds and Substrate

10 μL of 10 mM compounds stocks (1000×) dissolved in DMSO were pipetted into the appropriate wells on the 384 LDV plate (Labcyte; cat. no.: LP-0200) and the appropriate volumes of DMSO solvent were included onto an intermediate plate to further dilute the compound stocks to complete the 9-pt dose curve (final top concentration of 10 μM). 10 μL per well of 5 mM estradiol-d₂ (1000×) (Cayman; item no.: 9002846) (5 μM final concentration) dissolved in DMSO was seeded into the appropriate number of wells on the same source plate as the compounds. The Echo 650 (Labcyte) was used for compound dilutions and dispensing both the compounds and the substrate onto a dry 384-well black assay plate (Falcon/Corning; cat. no.:353962). Afterwards, cells were immediately counted and seeded onto the plate.

### Cell Treatment

Cells were cultured for expansion in EMEM (ATCC; cat. no.: 30-2003)+10% FBS (Hyclone; cat. no.: SH3007003)+1×PenStrep (Gibco; cat. no.: 15140-122). All media for stable cell line overexpressing HSD17013 also included 250 ng/mL zeocin (Invitrogen; cat. no.: 46-0509) for selection. To prepare cells for seeding into the 384-well assay plate, cells were trypsinized for 3 minutes in the incubator. The Trypsin-EDTA (0.25%) (Gibco; cat. no.: 25200056) was neutralized with EMEM complete culture medium warmed to 37° C. Cells were then centrifugated at 300×g for 5 minutes at room temperature. The cell pellet was resuspended in phenol red-free MEM (Gibco; cat. no.: 51200-038)+1% charcoal/dextran-treated FBS (Hyclone; cat. no.: SH3006803) and 1×PenStrep warmed to 37° C. Cells were then counted using the TC20 automated cell-counter (Bio-Rad) and disposable duel chamber counting slides (Bio-Rad; cat. no.: 1450016) for a cell density of 1.5E5 cells/mL to then seed into the assay plate at 7.5E3 cells/well with 50 μL of media per well.

### Collecting the Supernatant and Assessing Cytotoxicity

Prior to the 24-hour time point, a solution of 1% formic acid (10×) (Honeywell; cat. no.: 94318) in MEM phenol red-free MEM+1% charcoal/dextran-treated FBS and 1×PenStrep was made. ATPlite luminescent ATP detection reagent (PerkinElmer; cat. no.: 6016739) was prepared according to the manufacturer's instructions. A new 384-well Falcon plate (collection plate) was seeded with 2 μL per well of 10× formic acid and then 18 μL of cell supernatant was added onto the formic acid, resulting in a 20 μL per well solution of 0.1% formic acid (1×) in supernatant. The plate of collected supernatant was immediately frozen on dry ice before being stored at −80° C. The collection plate was shipped to Momentum Biotechnologies for analysis. The remaining supernatant in the assay plate was discarded and 25 μL of ATPlite reagent was added to each well. The plate was shaken for 2 minutes at room temperature before being read on the Envision plate reader (PerkinElmer) for luminescence signal.

Measuring Conversion of Estradiol-d₂ to Estrone-d₂ by RF-MS.

At Momentum Biotechnologies, samples were mixed with 2× volumes of cold acetonitrile, causing the proteins to precipitate and crash out of solution. The plates were then centrifuged at 3000×g for 12 minutes to pellet the crashed proteins to protect the instrument from clogs. The supernatant was then directly injected for analysis using the RF-MS system.

### Enzymatic Activity Calculation

The conversion (%) of Estradiol-d₂ to Estrone-d₂ after 24 hours was measured by RF-MS. Conversion indicates the amount of labelled estrone produced from estradiol. The calculation below was applied to every well to determine the conversion:

$$Conversion ( \% ) = \frac{Estrone - {d}_{2}}{[ Estrone - {d}_{2} ] + [ Estradiol - {d}_{2} ]} \times 100 \% \qquad [ 1 ]$$

To then scale the curves to measure the percent inhibition of HSD17β13 enzymatic activity by the inhibitors, the values of each well of treated cells was normalized to the wells with the corresponding untreated cells dosed only with DMSO vehicle and Estradiol-d₂. The equation below was used to measure enzymatic activity (0) of the stable cell line verexpressing HSD17β13:

$$Enzymatic activity ( \% ) = \frac{{( Conversion )}_{Treated}}{{( Conversion )}_{Untreated}} \times 100 \% . \qquad [ 2 ]$$

By using the above method, the inhibition of 17β-HSD13 was evaluated for the compounds of Formula (I). IC₅₀ ranges are as follows: A: IC₅₀<0.100 μM; B: 0.100 μM<IC₅₀<0.500 μM; C: 0.500 μM<IC₅₀<1 μM; D:1 μM<IC₅₀<100 μM; E: IC₅₀>100 μM.

| Example | IC₅₀ |
|---|---|
| 1 | D |
| 2 | D |
| 3 | D |
| 4 | D |
| 5 | D |
| 6 | D |
| 7 | D |
| 8 | D |
| 9 | D |
| 10 | B |
| 11 | C |
| 12 | D |
| 13 | D |
| 14 | D |
| 15 | D |
| 16 | D |
| 17 | D |
| 18 | B |
| 19 | B |
| 20 | D |
| 21 | D |
| 22 | D |
| 23 | D |
| 24 | A |
| 25 | B |
| 26 | A |
| 27 | A |
| 28 | D |
| 29 | C |
| 30 | D |
| 31 | D |
| 32 | B |
| 33 | C |
| 34 | D |
| 35 | D |
| 36 | D |
| 37 | D |
| 38 | D |
| 39 | A |
| 40 | A |
| 41 | A |
| 42 | A |
| 43 | A |
| 44 | A |
| 45 | B |
| 46 | D |
| 47 | A |
| 48 | A |
| 49 | A |
| 50 | A |
| 51 | C |
| 52 | A |
| 53 | A |
| 54 | B |
| 55 | A |
| 56 | A |
| 57 | A |
| 58 | A |
| 59 | A |
| 60 | A |
| 61 | A |
| 62 | A |
| 63 | A |
| 64 | A |
| 65 | A |
| 66 | A |
| 67 | A |
| 68 | A |
| 69 | A |
| 70 | A |
| 71 | A |
| 72 | A |
| 73 | A |
| 74 | A |
| 75 | A |
| 76 | A |
| 77 | A |
| 78 | A |
| 79 | A |
| 80 | A |
| 81 | A |
| 82 | A |
| 83 | A |
| 84 | A |
| 85 | A |
| 86 | A |
| 87 | A |
| 88 | A |
| 89 | A |
| 90 | A |
| 91 | A |
| 92 | A |
| 93 | A |
| 94 | A |
| 95 | A |
| 96 | A |
| 97 | A |
| 98 | A |
| 99 | A |
| 100 | A |
| 101 | A |
| 102 | A |
| 103 | A |
| 104 | A |
| 105 | A |
| 106 | A |
| 107 | B |
| 108 | A |
| 109 | C |
| 110 | A |
| 111 | A |
| 112 | A |
| 113 | A |
| 114 | A |
| 115 | A |
| 116 | A |
| 117 | B |
| 118 | B |
| 119 | A |
| 120 | A |
| 121 | B |
| 122 | A |
| 123 | A |
| 124 | B |
| 125 | A |
| 126 | B |
| 127 | A |
| 128 | C |
| 129 | A |
| 130 | A |
| 131 | A |
| 132 | A |
| 133 | A |
| 134 | A |
| 135 | A |
| 136 | A |
| 137 | A |
| 138 | A |
| 139 | A |
| 140 | A |
| 141 | A |
| 142 | A |
| 143 | A |
| 144 | A |
| 145 | A |
| 146 | A |
| 147 | A |
| 148 | A |
| 149 | A |
| 150 | C |
| 151 | A |
| 152 | A |
| 153 | A |
| 154 | A |
| 155 | A |
| 156 | A |
| 157 | B |
| 158 | A |
| 159 | A |
| 160 | A |
| 161 | A |
| 162 | A |
| 163 | A |
| 164 | A |
| 165 | A |
| 166 | A |
| 167 | A |
| 168 | A |
| 169 | A |
| 170 | A |
| 171 | A |
| 172 | A |
| 173 | A |
| 174 | A |
| 175 | A |
| 176 | A |
| 177 | A |
| 178 | A |
| 179 | A |
| 180 | A |
| 181 | A |
| 182 | A |
| 183 | A |
| 184 | A |
| 185 | A |
| 186 | A |
| 187 | A |
| 188 | B |
| 189 | A |
| 190 | A |
| 191 | A |
| 192 | A |
| 193 | A |
| 194 | C |
| 195 | A |
| 196 | A |
| 197 | A |
| 198 | A |
| 199 | A |
| 200 | A |
| 201 | A |
| 202 | A |
| 203 | B |
| 204 | A |
| 205 | A |
| 206 | B |
| 207 | A |
| 208 | A |
| 209 | A |
| 210 | A |
| 211 | A |
| 212 | A |
| 213 | D |
| 214 | A |
| 215 | A |
| 216 | A |
| 217 | A |
| 218 | A |
| 219 | A |
| 220 | A |
| 221 | B |
| 222 | B |
| 223 | A |

### HSD17B13 Inhibitor Decreases Fibrotic Markers in Mouse Livers Fed a Choline Deficient Hight Fat (CDAA-HF) Diet

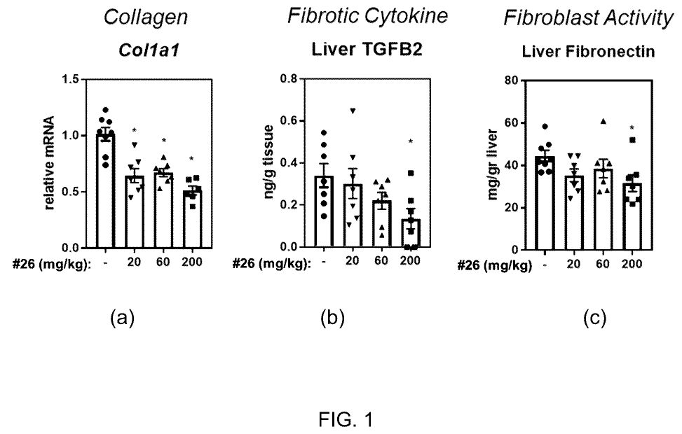

The choline deficient amino acid defined diet (CDAA) is a well-characterized model of non-metabolic associated liver inflammation and fibrosis. The CDAA-HF mouse model is commonly used to preclinically evaluate potential therapeutic targets for non-alcoholic steatohepatitis (NASH). To investigate the effect of Compound 26 (Example 26; 20, 60, and 200 mg/kg, oral gavage, daily) on fibrotic gene and protein parameters, 10 week-old male C57BL/6J mice were fed CDAA-HF (choline deficient, L-amino acid defined, high fat diet; A16092201) for 4 weeks, with Compound 26 treatment only during the final week of the study. On the last day of the study, mice were euthanized by CO₂ asphyxiation for tissue collection, and blood was collected by cardiac puncture. Livers were rapidly removed and frozen in liquid nitrogen for downstream mRNA and protein isolation. FIG. 1 shows Compound 26 significantly decreases liver collagen mRNA, the fibrotic TGFB2 cytokine, and the fibroblast activation marker fibronectin. Statistical analysis was performed with One Way ANOVA. *p<0.05 vs vehicle treated (FIG. 1). Col1a1=Collagen; TGFB2=transforming growth factor beta 2. All procedures performed on animals were in accordance with regulations and established guidelines and were reviewed and approved by an Institutional Animal Care and Use Committee or through an ethical review process.

### HSD17B13 Inhibitor Decreases Fibrotic Markers in Precision Cut Lung Slice (PCLS) Culture

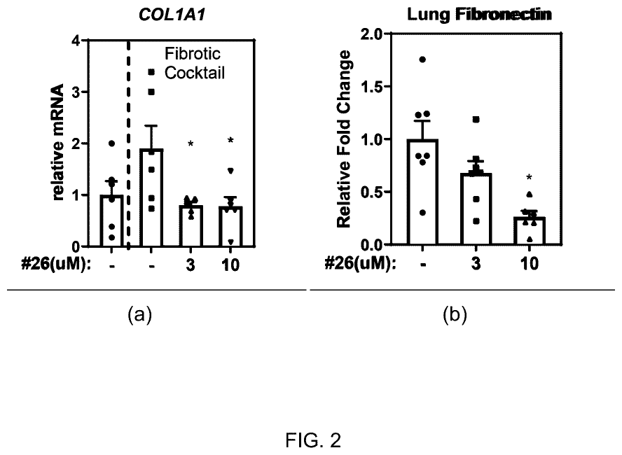

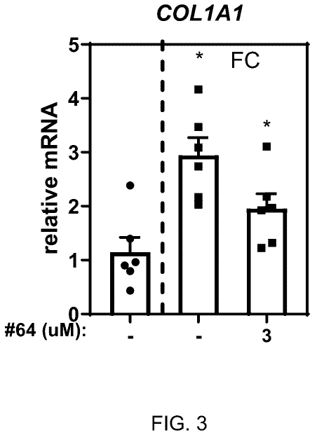

Human PCLS were generated from lung explants of patients with pulmonary fibrosis (donor ID #221028) or normal donor (donor ID #220730). Cryopreserved hPCLSs were obtained from Institute for In Vitro Sciences (IIVS) and maintained with air-liquid interface culture approach under 37° C., 5% CO2 and 95% humidity. hPCLS were cultured in DMEM-F12 (Invitrogen, Cat No. 11330032) containing 0.2% primocin (Invivogen, Cat No. ant-pm-1) and 1% insulin-transferrin-selenium (ITS-G; Invitrogen, Cat No. 41400045), after initial acclimation in acclimation media, including culture media supplemented with 1% antibiotic antimycotic solution (Invitrogen, Cat No. 15240062), 2 mM Hydrocortisone (Millipore Sigma,) and 5 mg/ml 2-phospho-L-abscorbic acid trisodium salt (Millipore Sigma, Cat No. 49752-10G) for 3 days. Slices of tissue were flatly grown in the tissue culture inserts with 0.4 μm pores of a 12-well plate (StemCell Technologies, Cat No. 1001027), supplemented with 100 μl culture media in the apical side containing PCLSs, and 900 μL culture media in the basal compartment. Media were changed every 48 hours. hPCLS were treated and randomly assigned in replicates of 6-8 per condition 7 days after recovery from cryopreservation. Supernatants were collected at day 0, 2, 4, 6 after treatment for secreted protein analysis. IPF donor PCLS were collected at day 8 for subsequent RNA isolation and gene expression quantification. Normal donor PCLS were treated for 8 days with a control cocktail (CC) including all vehicles or a pro-fibrotic cocktail (FC) consisting of transforming growth factor-μ(TGF-β) (5 ng/ml, Bio-Techne), platelet-derived growth factor-AB (PDGF-AB) (10 ng/ml, Thermo Fisher), tumor necrosis factor-α (TNF-α) (10 ng/ml, Bio-Techne), and lysophosphatidic acid (LPA) (5 μM, Cayman chemical) as described before. Compound 26 was used as the HSD17B13 inhibitor. FIG. 2 shows the anti-fibrotic properties of Compound 26 in human precision cut lung slices from (a) a normal donor treated with fibrotic cocktail, or (b) from a donor with idiopathic pulmonary fibrosis. Statistical analysis was performed with One Way ANOVA. *p<0.05 vs vehicle treated. COL1A1=Collagen. HSD17B13 inhibitor decreased gene (COL1A1) expression and secreted protein markers (fibronectin) of fibrosis in IPF donor PCLS and FC-treated normal donor PCLS (FIG. 2). FIG. 3 shows the anti-fibrotic properties of Compound 64 (Example 64) in human precision cut lung slices from a normal donor treated with fibrotic cocktail with decreased COL1A1 expression.

While this invention has been particularly shown and described with references to preferred embodiments thereof, it will be understood by those skilled in the art that various changes in form and details may be made therein without departing from the scope of the invention encompassed by the appended claims.

1. A compound represented by Formula I or a pharmaceutically acceptable salt, or ester thereof: 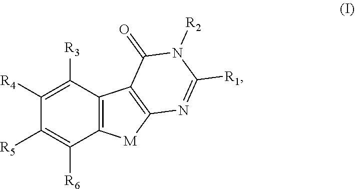
wherein,
M is S, SO, SO₂, O, or NR₇;
R₁ and R₂ are each independently selected from the group consisting of:
1) Hydrogen;
2) Optionally substituted —C₁-C₈ alkyl;
3) Optionally substituted —C₂-C₅ alkenyl;
4) Optionally substituted —C₂-C₅ alkynyl;
5) Optionally substituted —C₃-C₈ cycloalkyl;
6) Optionally substituted aryl;
7) Optionally substituted arylalkyl;
8) Optionally substituted 3- to 8-membered heterocycloalkyl;
9) Optionally substituted heteroaryl; and
10) Optionally substituted heteroarylalkyl;
R₃, R₄, R₅, and R₆ are each independently selected from the group consisting of hydrogen, halogen, —CN, —OR₉, —SR₉, —B(OR₁₃)₂, —SO₂R₁₃, —SO₂OR₁₃, —OSO₂OR₁₃, —P(O)(OR₁₃)₂, —C(O)R₇, —C(O)OR₇, —NR₇R₈, —NR₇(COR₈), —NR₇C(O)OR₈, —N(COR₈)₂, —NR₇SO₂R₈, —C(O)NR₇R₈, optionally substituted —C₁-C₈ alkyl, optionally substituted —C₂-C₅ alkenyl, optionally substituted aryl, and optionally substituted heteroaryl;
alternatively, R₅ and R₆ are taken together with the carbon atoms to which they are attached to form an optionally substituted carbocyclic or heterocyclic ring;
alternatively, R₄ and R₅ are taken together with the carbon atoms to which they are attached to form an optionally substituted carbocyclic or heterocyclic ring;
alternatively, R₃ and R₄ are taken together with the carbon atoms to which they are attached to form an optionally substituted carbocyclic or heterocyclic ring;
each R₇ and R₈ is independently selected from the group consisting of:
1) Hydrogen;
2) Optionally substituted —C₁-C₈ alkyl;
3) Optionally substituted —C₂-C₈ alkenyl;
4) Optionally substituted —C₂-C₈ alkynyl;
5) Optionally substituted —C₃-C₈ cycloalkyl;
6) Optionally substituted 3- to 8-membered heterocycloalkyl;
7) Optionally substituted aryl;
8) Optionally substituted arylalkyl;
9) Optionally substituted heteroaryl; and
10) Optionally substituted heteroarylalkyl;
alternatively, R₇ and R₈ are taken together with the nitrogen atom to which they are attached to form an optionally substituted heterocyclic ring;
R₉ is selected from the group consisting of:
1) Hydrogen;
2) Optionally substituted —C₁-C₈ alkyl;
3) Optionally substituted —C₂-C₈ alkenyl;
4) Optionally substituted —C₂-C₈ alkynyl;
5) Optionally substituted —C₃-C₈ cycloalkyl;
6) Optionally substituted 3- to 8-membered heterocycloalkyl;
7) Optionally substituted aryl;
8) Optionally substituted arylalkyl;
9) Optionally substituted heteroaryl;
10) Optionally substituted heteroarylalkyl;
11) —C(O)R₁₁;
12) —C(O)NR₁₁R₁₂;
13) —C(O)OR₁₁;
14) —P(O)(OR₁₃)₂; and
15) —P(O)(OR₁₃)(NR₁₁R₁₂);
R₁₁ and R₁₂ are each independently selected from the group consisting of:
1) Hydrogen;
2) Optionally substituted —C₁-C₈ alkyl;
3) Optionally substituted —C₂-C₈ alkenyl;
4) Optionally substituted —C₂-C₈ alkynyl;
5) Optionally substituted —C₃-C₈ cycloalkyl;
6) Optionally substituted 3- to 8-membered heterocycloalkyl;
7) Optionally substituted aryl;
8) Optionally substituted arylalkyl;
9) Optionally substituted heteroaryl; and
10) Optionally substituted heteroarylalkyl;
and R₁₃ is hydrogen, optionally substituted —C₁-C₈ alkyl, or Na⁺;
provided that R₅ and R₆ are not hydrogen.

2. The compound of claim 1, wherein R₁ is optionally substituted aryl or optionally substituted heteroaryl, and R₂ is optionally substituted heteroaryl.

3. The compound of claim 1, represented by Formula (XIV), or a pharmaceutically acceptable salt thereof: 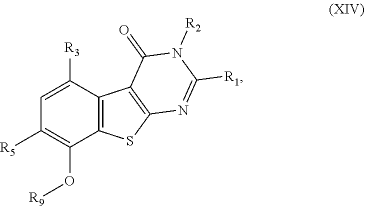
wherein, R₁, R₂, R₃, R₅, and R₉ are as defined in claim 1.

4. The compound of claim 3, wherein R₁ is optionally substituted aryl or optionally substituted heteroaryl, and R₂ is optionally substituted heteroaryl.

5. The compound of claim 1, represented by Formula (XX), or a pharmaceutically acceptable salt thereof: 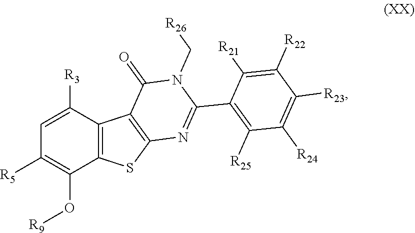
wherein each R₂₁, R₂₂, R₂₃, R₂₄, or R₂₅ is independently hydrogen, halogen, optionally substituted —C₁-C₆ alkyl, optionally substituted —C₁-C₆ alkoxyl, or optionally substituted —C₃-C₈-cycloalkyl; R₂₆ is optionally substituted aryl, optionally substituted heteroaryl, optionally substituted heteroarylalkyl, optionally substituted —C₃-C₈-cycloalkyl, optionally substituted —C₁-C₆ alkyl, —NR₁₁R₁₂, —CH₂NR₁₁R₁₂, —CH₂NR₁₁C(O) R₁₂, or I, or 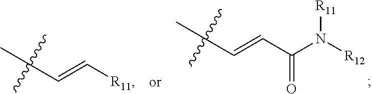
and R₃, R₅, R₁₁, R₁₂, and R₉ are as defined in claim 1.

6. The compound of claim 1, represented by one of Formulae (XXXII-1)˜(XXXII-3), or a pharmaceutically acceptable salt or ester thereof: 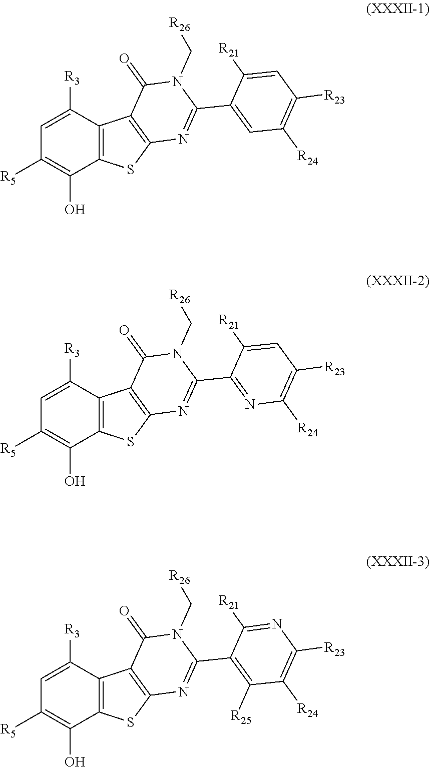
wherein each R₂₁, R₂₃, R₂₄, or R₂₅ is independently hydrogen, halogen, optionally substituted —C₁-C₆ alkyl, optionally substituted —C₁-C₆ alkoxyl, or optionally substituted —C₃-C₈-cycloalkyl; R₂₆ is optionally substituted aryl, optionally substituted heteroaryl, optionally substituted heteroarylalkyl, optionally substituted —C₃-C₈-cycloalkyl, optionally substituted —C₁-C₆ alkyl, —NR₁₁R₁₂, —CH₂NR₁₁R₁₂, —CH₂NR₁₁C(O) R₁₂, or 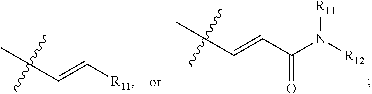
and R₃, R₅, R₁₁, and R₁₂ are as defined in claim 1.

7. The compound of claim 1, represented by one of Formulae (XXXIII-1)˜(XXXIII-3), or a pharmaceutically acceptable salt or ester thereof: 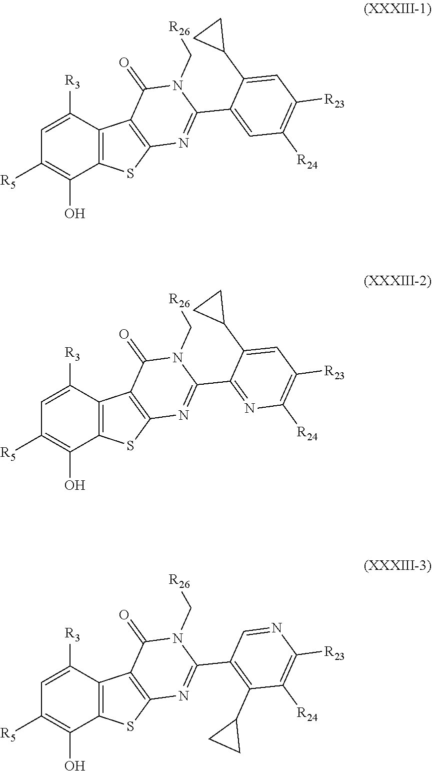
wherein each R₂₃ or R₂₄ is independently hydrogen, halogen, optionally substituted —C₁-C₆ alkyl, optionally substituted —C₁-C₆ alkoxyl, or optionally substituted —C₃-C₈-cycloalkyl; R₂₆ is optionally substituted aryl, optionally substituted heteroaryl, optionally substituted heteroarylalkyl, optionally substituted —C₃-C₈-cycloalkyl, optionally substituted —C₁-C₆ alkyl, —NR₁₁R₁₂, —CH₂NR₁₁R₁₂, —CH₂NR₁₁C(O) R₁₂, or 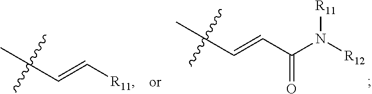
and R₃, R₅, R₁₁, and R₁₂ are as defined in claim 1.

8. The compound of claim 1, represented by one of Formulae (XXXVI-1)˜(XXXVI-10), or a pharmaceutically acceptable salt or ester thereof: 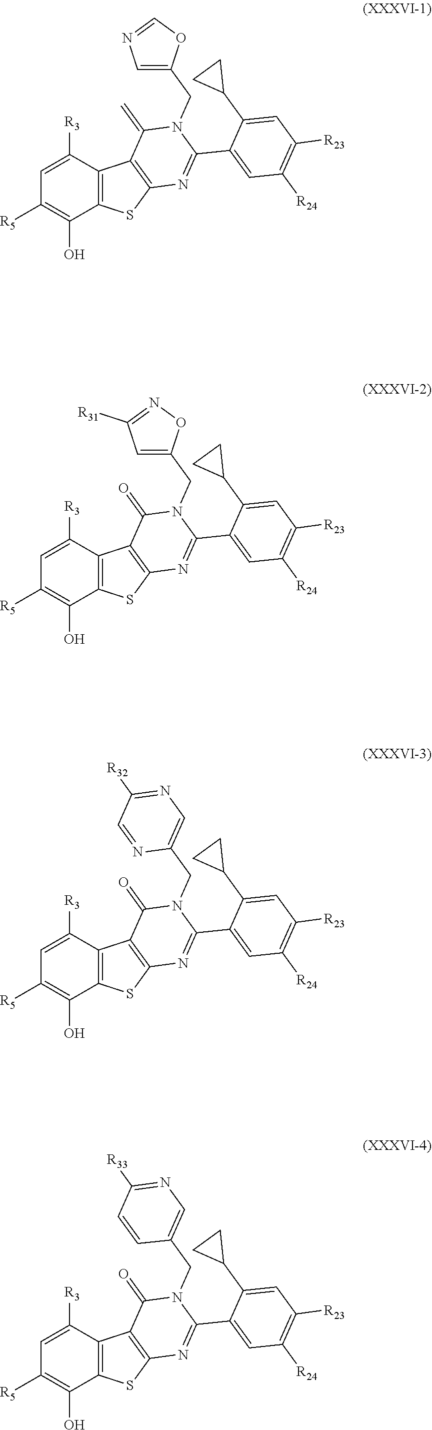 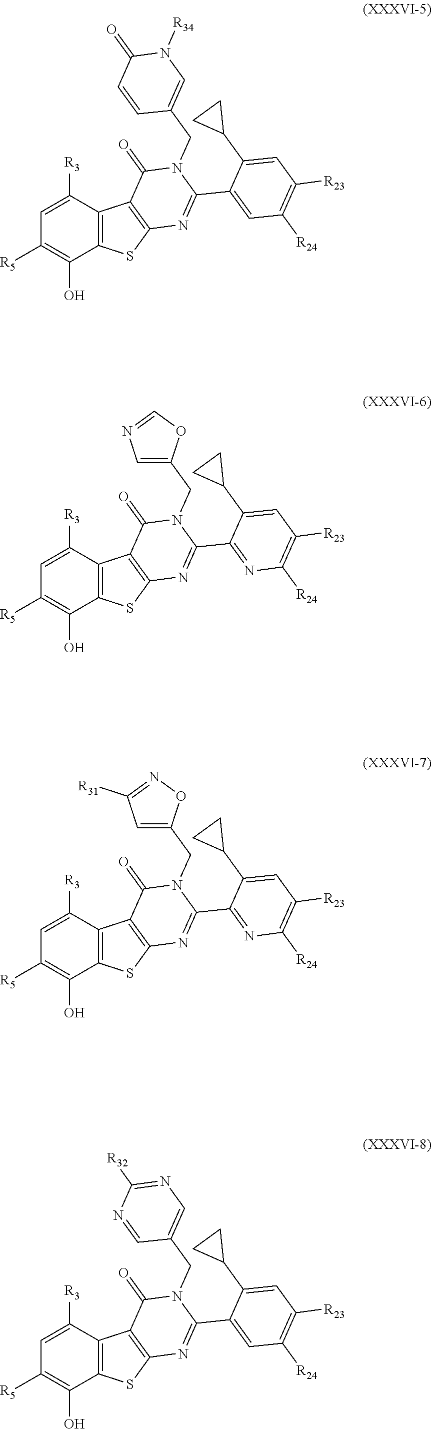 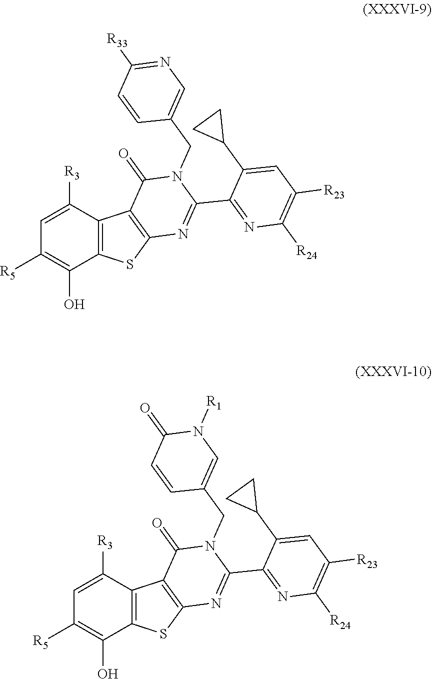
wherein R₂₃ and R₂₄ are independently hydrogen, halogen, optionally substituted —C₁-C₆ alkyl, optionally substituted —C₁-C₆ alkoxyl, or optionally substituted —C₃-C₈-cycloalkyl; R₃₁, R₃₂, R₃₃, or R₃₄ are each independently hydrogen, halogen, optionally substituted —C₁-C₆ alkyl, optionally substituted —C₁-C₆ alkoxyl, or optionally substituted —C₃-C₈-cycloalkyl; R₅ and R₃ are as defined in claim 1.

9. The compound of claim 1, represented by one of Formulae (XXXVIII-1)˜(XXXVIII-10), or a pharmaceutically acceptable salt or ester thereof: 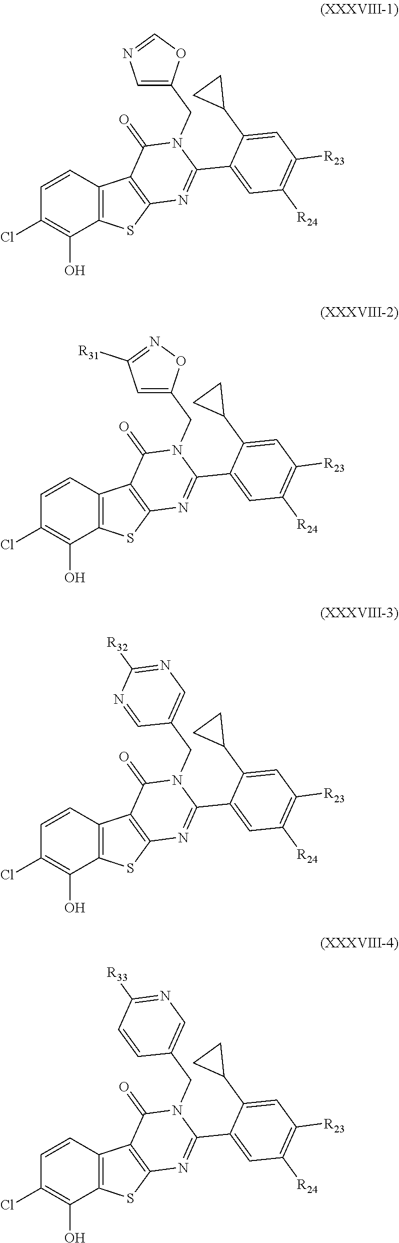 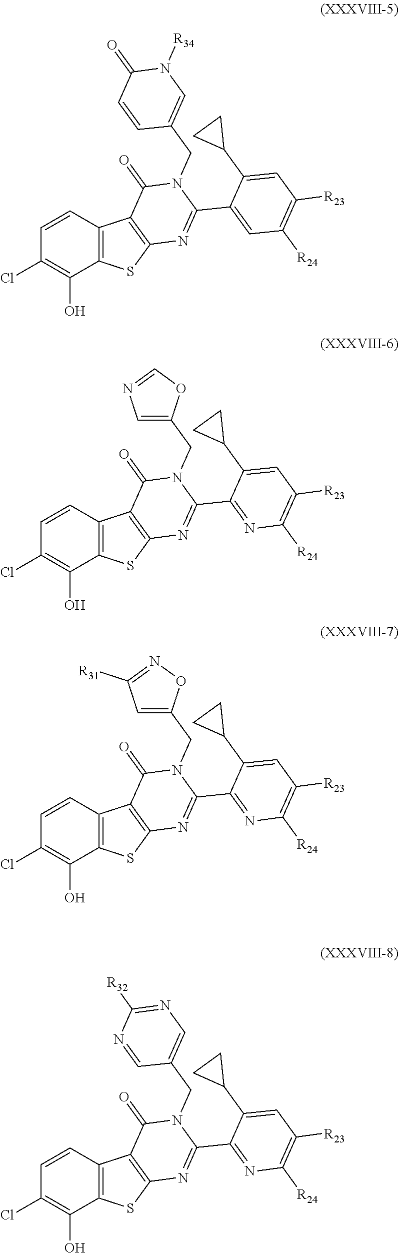 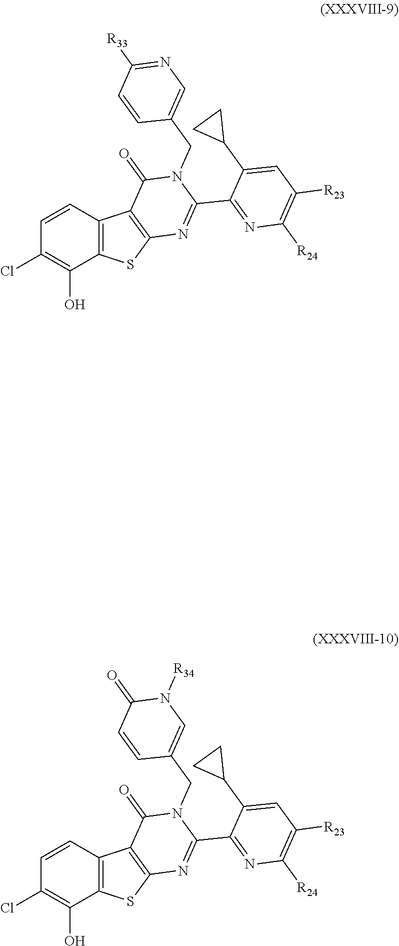
wherein each R₂₃ or R₂₄ is independently hydrogen, halogen, optionally substituted —C₁-C₆ alkyl, optionally substituted —C₁-C₆ alkoxyl, or optionally substituted —C₃-C₈-cycloalkyl; each R₃₁, R₃₂, R₃₃, or R₃₄ is independently hydrogen, halogen, optionally substituted —C₁-C₆ alkyl, optionally substituted —C₁-C₆ alkoxyl, or optionally substituted —C₃-C₈-cycloalkyl.

10. The compound of claim 1, selected from the compounds set forth below or a pharmaceutically acceptable salt thereof:

| Com- | |
|---|---|
| pound | Structure |
| 1 | 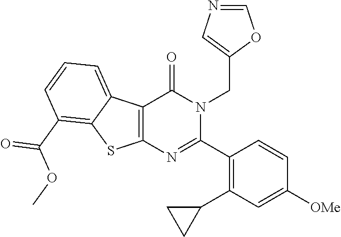 [SMILES: COC(=O)c1cccc2c1sc1nc(-c3ccc(OC)cc3C3CC3)n(Cc3cnco3)c(=O)c12] |
| 2 | 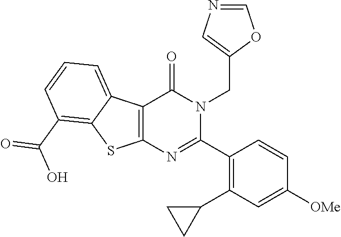 [SMILES: COc1ccc(-c2nc3sc4c(C(=O)O)cccc4c3c(=O)n2Cc2cnco2)c(C2CC2)c1] |
| 3 | 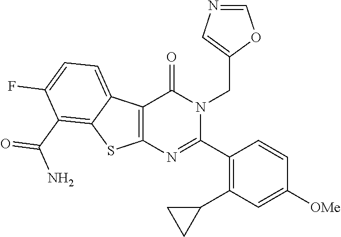 [SMILES: COc1ccc(-c2nc3sc4c(C(N)=O)c(F)ccc4c3c(=O)n2Cc2cnco2)c(C2CC2)c1] |
| 4 | 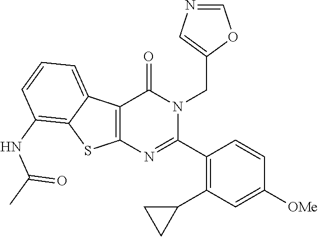 [SMILES: COc1ccc(-c2nc3sc4c(NC(C)=O)cccc4c3c(=O)n2Cc2cnco2)c(C2CC2)c1] |
| 5 | 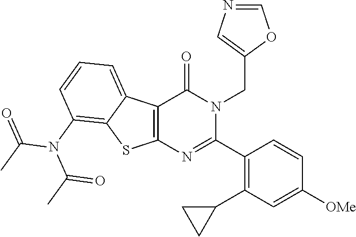 [SMILES: COc1ccc(-c2nc3sc4c(N(C(C)=O)C(C)=O)cccc4c3c(=O)n2Cc2cnco2)c(C2CC2)c1] |
| 6 | 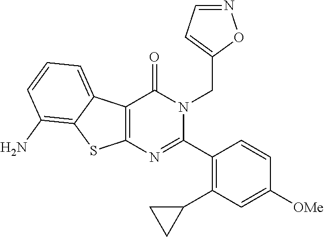 [SMILES: COc1ccc(-c2nc3sc4c(N)cccc4c3c(=O)n2Cc2ccno2)c(C2CC2)c1] |
| 7 | 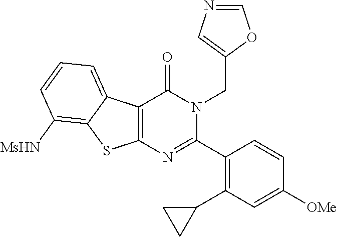 |
| 8 | 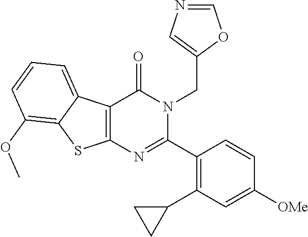 [SMILES: COc1ccc(-c2nc3sc4c(OC)cccc4c3c(=O)n2Cc2cnco2)c(C2CC2)c1] |
| 9 | 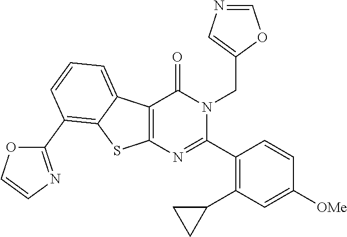 [SMILES: COc1ccc(-c2nc3sc4c(-c5ncco5)cccc4c3c(=O)n2Cc2cnco2)c(C2CC2)c1] |
| 10 | 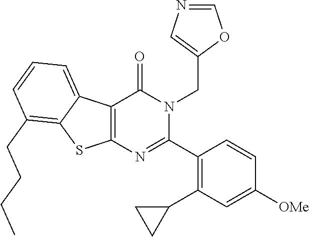 [SMILES: CCCCc1cccc2c1sc1nc(-c3ccc(OC)cc3C3CC3)n(Cc3cnco3)c(=O)c12] |
| 11 | 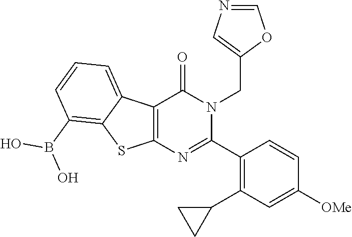 [SMILES: COc1ccc(-c2nc3sc4c(B(O)O)cccc4c3c(=O)n2Cc2cnco2)c(C2CC2)c1] |
| 12 | 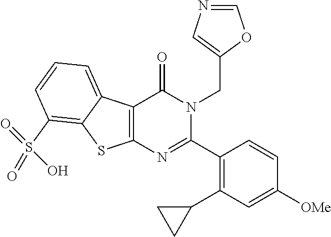 [SMILES: COc1ccc(-c2nc3sc4c(S(=O)(=O)O)cccc4c3c(=O)n2Cc2cnco2)c(C2CC2)c1] |
| 13 | 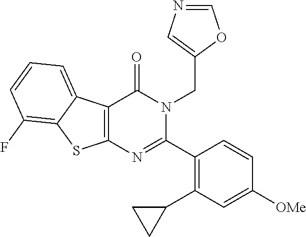 [SMILES: COc1ccc(-c2nc3sc4c(F)cccc4c3c(=O)n2Cc2cnco2)c(C2CC2)c1] |
| 14 | 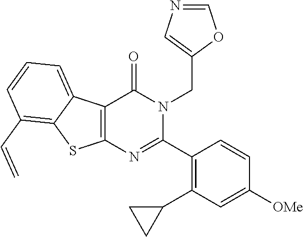 [SMILES: C=Cc1cccc2c1sc1nc(-c3ccc(OC)cc3C3CC3)n(Cc3cnco3)c(=O)c12] |
| 15 | 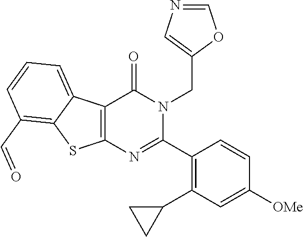 [SMILES: COc1ccc(-c2nc3sc4c(C=O)cccc4c3c(=O)n2Cc2cnco2)c(C2CC2)c1] |
| 16 | 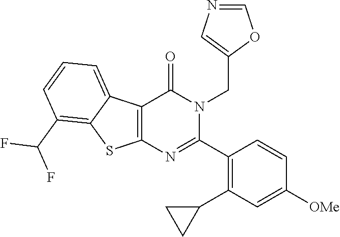 [SMILES: COc1ccc(-c2nc3sc4c(C(F)F)cccc4c3c(=O)n2Cc2cnco2)c(C2CC2)c1] |
| 17 | 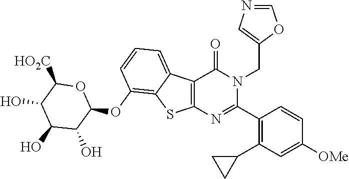 [SMILES: COc1ccc(-c2nc3sc4c(O[C@@H]5O[C@H](C(=O)O)[C@@H](O)[C@H](O)[C@H]5O)cccc4c3c(=O)n2Cc2cnco2)c(C2CC2)c1] |
| 18 | 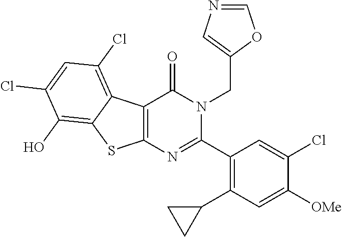 [SMILES: COc1cc(C2CC2)c(-c2nc3sc4c(O)c(Cl)cc(Cl)c4c3c(=O)n2Cc2cnco2)cc1Cl] |
| 19 | 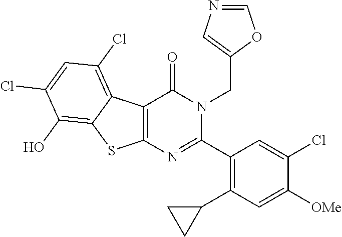 [SMILES: COc1cc(C2CC2)c(-c2nc3sc4c(O)c(Cl)cc(Cl)c4c3c(=O)n2Cc2cnco2)cc1Cl] |
| 20 | 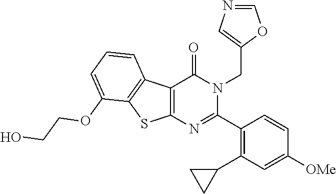 [SMILES: COc1ccc(-c2nc3sc4c(OCCO)cccc4c3c(=O)n2Cc2cnco2)c(C2CC2)c1] |
| 21 | 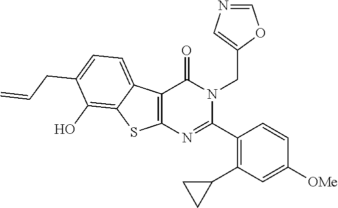 [SMILES: C=CCc1ccc2c(sc3nc(-c4ccc(OC)cc4C4CC4)n(Cc4cnco4)c(=O)c32)c1O] |
| 22 | 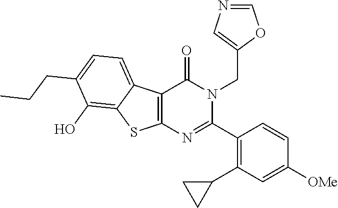 [SMILES: CCCc1ccc2c(sc3nc(-c4ccc(OC)cc4C4CC4)n(Cc4cnco4)c(=O)c32)c1O] |
| 23 | 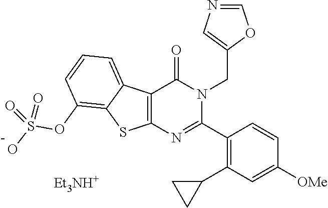 [SMILES: "Et3NH+" ; COc1ccc(-c2nc3sc4c(OS(=O)(=O)O)cccc4c3c(=O)n2Cc2cnco2)c(C2CC2)c1] |
| 24 | 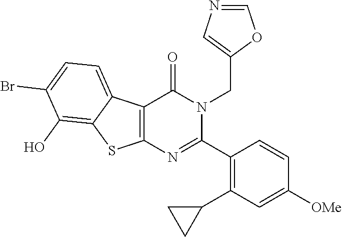 [SMILES: COc1ccc(-c2nc3sc4c(O)c(Br)ccc4c3c(=O)n2Cc2cnco2)c(C2CC2)c1] |
| 25 | 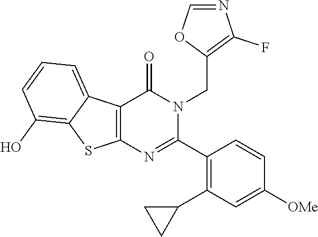 [SMILES: COc1ccc(-c2nc3sc4c(O)cccc4c3c(=O)n2Cc2ocnc2F)c(C2CC2)c1] |
| 26 | 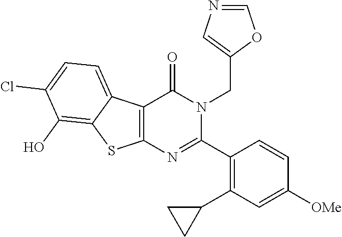 [SMILES: COc1ccc(-c2nc3sc4c(O)c(Cl)ccc4c3c(=O)n2Cc2cnco2)c(C2CC2)c1] |
| 27 | 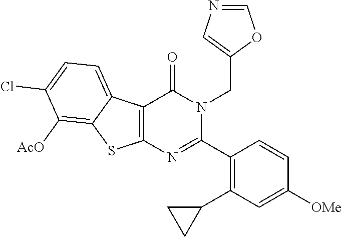 [SMILES: COc1ccc(-c2nc3sc4c(OC(C)=O)c(Cl)ccc4c3c(=O)n2Cc2cnco2)c(C2CC2)c1] |
| 28 | 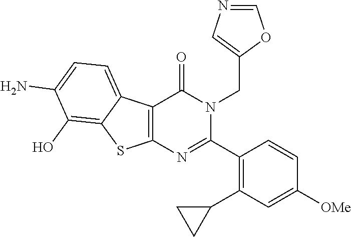 [SMILES: COc1ccc(-c2nc3sc4c(O)c(N)ccc4c3c(=O)n2Cc2cnco2)c(C2CC2)c1] |
| 29 | 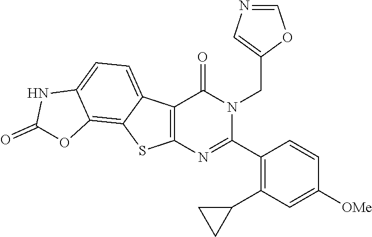 [SMILES: COc1ccc(-c2nc3sc4c(ccc5[nH]c(=O)oc54)c3c(=O)n2Cc2cnco2)c(C2CC2)c1] |
| 30 | 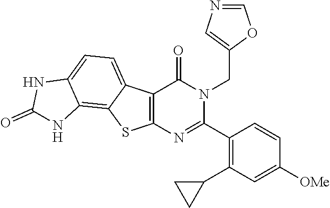 [SMILES: COc1ccc(-c2nc3sc4c(ccc5[nH]c(=O)[nH]c54)c3c(=O)n2Cc2cnco2)c(C2CC2)c1] |
| 31 | 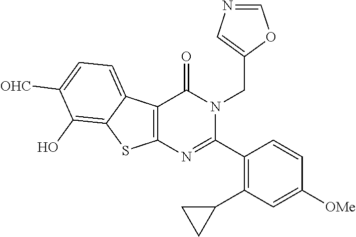 [SMILES: COc1ccc(-c2nc3sc4c(O)c(C=O)ccc4c3c(=O)n2Cc2cnco2)c(C2CC2)c1] |
| 32 | 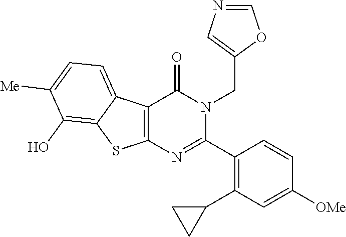 [SMILES: COc1ccc(-c2nc3sc4c(O)c(C)ccc4c3c(=O)n2Cc2cnco2)c(C2CC2)c1] |
| 33 | 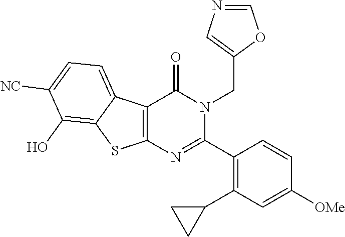 [SMILES: COc1ccc(-c2nc3sc4c(O)c(C#N)ccc4c3c(=O)n2Cc2cnco2)c(C2CC2)c1] |
| 34 | 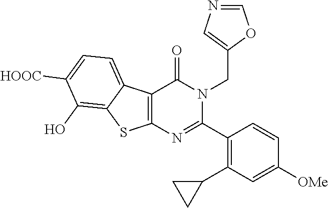 [SMILES: COc1ccc(-c2nc3sc4c(O)c(C(=O)O)ccc4c3c(=O)n2Cc2cnco2)c(C2CC2)c1] |
| 35 | 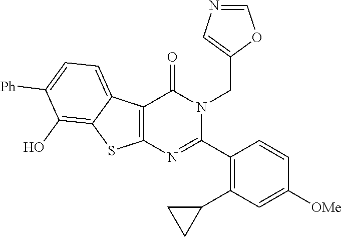 [SMILES: COc1ccc(-c2nc3sc4c(O)c(-c5ccccc5)ccc4c3c(=O)n2Cc2cnco2)c(C2CC2)c1] |
| 36 | 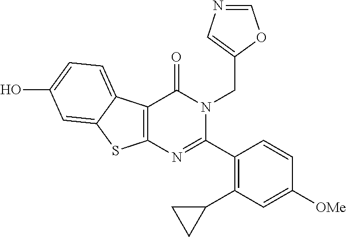 [SMILES: COc1ccc(-c2nc3sc4cc(O)ccc4c3c(=O)n2Cc2cnco2)c(C2CC2)c1] |
| 37 | 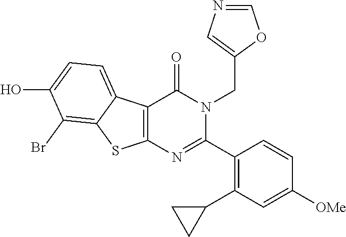 [SMILES: COc1ccc(-c2nc3sc4c(Br)c(O)ccc4c3c(=O)n2Cc2cnco2)c(C2CC2)c1] |
| 38 | 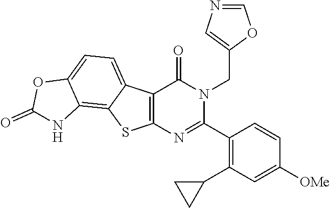 [SMILES: COc1ccc(-c2nc3sc4c(ccc5oc(=O)[nH]c54)c3c(=O)n2Cc2cnco2)c(C2CC2)c1] |
| 39 | 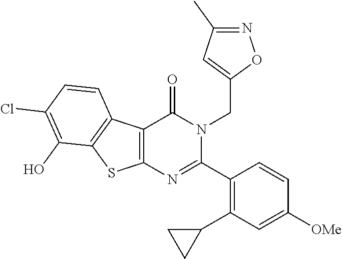 [SMILES: COc1ccc(-c2nc3sc4c(O)c(Cl)ccc4c3c(=O)n2Cc2cc(C)no2)c(C2CC2)c1] |
| 40 | 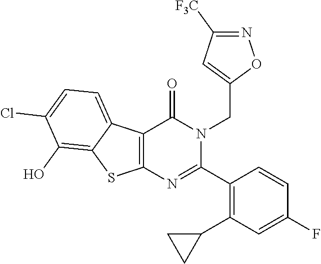 [SMILES: O=c1c2c(nc(-c3ccc(F)cc3C3CC3)n1Cc1cc(C(F)(F)F)no1)sc1c(O)c(Cl)ccc12] |
| 41 | 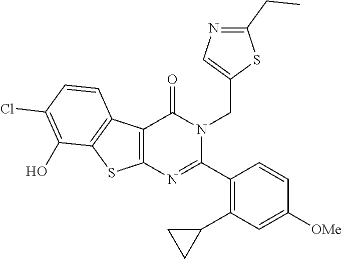 [SMILES: CCc1ncc(Cn2c(-c3ccc(OC)cc3C3CC3)nc3sc4c(O)c(Cl)ccc4c3c2=O)s1] |
| 42 | 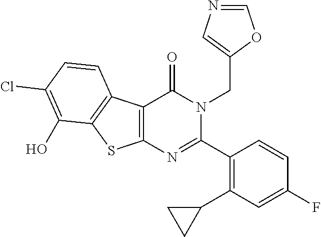 [SMILES: O=c1c2c(nc(-c3ccc(F)cc3C3CC3)n1Cc1cnco1)sc1c(O)c(Cl)ccc12] |
| 43 | 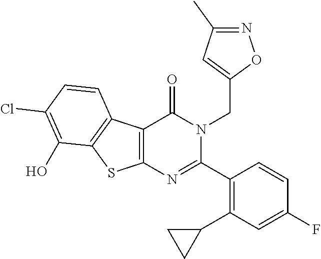 [SMILES: Cc1cc(Cn2c(-c3ccc(F)cc3C3CC3)nc3sc4c(O)c(Cl)ccc4c3c2=O)on1] |
| 44 | 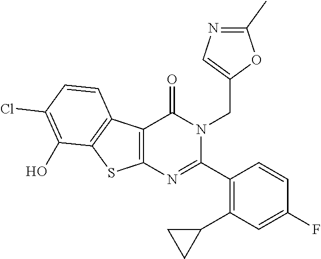 [SMILES: Cc1ncc(Cn2c(-c3ccc(F)cc3C3CC3)nc3sc4c(O)c(Cl)ccc4c3c2=O)o1] |
| 45 | 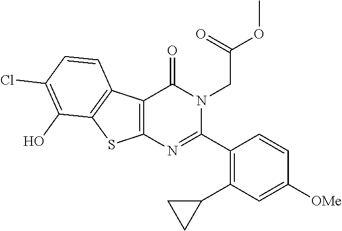 [SMILES: COC(=O)Cn1c(-c2ccc(OC)cc2C2CC2)nc2sc3c(O)c(Cl)ccc3c2c1=O] |
| 46 | 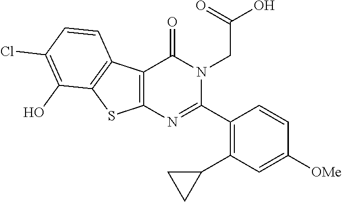 [SMILES: COc1ccc(-c2nc3sc4c(O)c(Cl)ccc4c3c(=O)n2CC(=O)O)c(C2CC2)c1] |
| 47 | 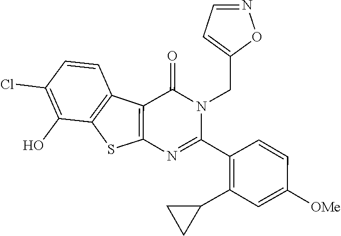 [SMILES: COc1ccc(-c2nc3sc4c(O)c(Cl)ccc4c3c(=O)n2Cc2ccno2)c(C2CC2)c1] |
| 48 | 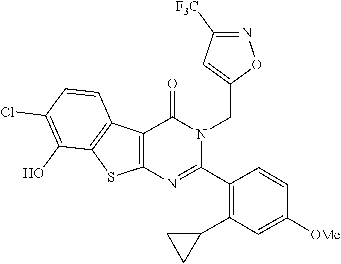 [SMILES: COc1ccc(-c2nc3sc4c(O)c(Cl)ccc4c3c(=O)n2Cc2cc(C(F)(F)F)no2)c(C2CC2)c1] |
| 49 | 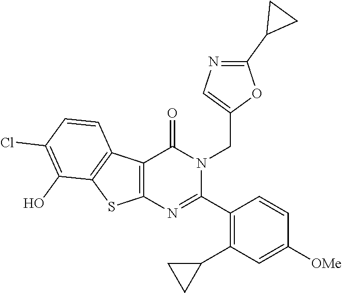 [SMILES: COc1ccc(-c2nc3sc4c(O)c(Cl)ccc4c3c(=O)n2Cc2cnc(C3CC3)o2)c(C2CC2)c1] |
| 50 | 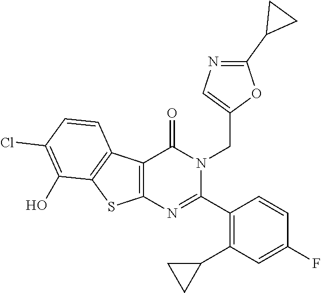 [SMILES: O=c1c2c(nc(-c3ccc(F)cc3C3CC3)n1Cc1cnc(C3CC3)o1)sc1c(O)c(Cl)ccc12] |
| 51 | 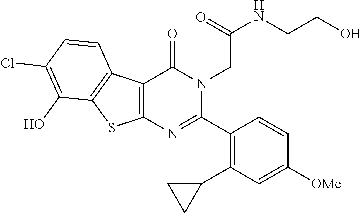 [SMILES: COc1ccc(-c2nc3sc4c(O)c(Cl)ccc4c3c(=O)n2CC(=O)NCCO)c(C2CC2)c1] |
| 52 | 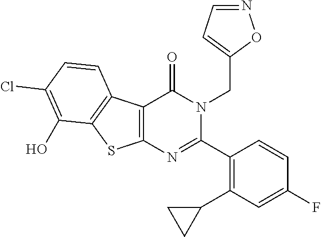 [SMILES: O=c1c2c(nc(-c3ccc(F)cc3C3CC3)n1Cc1ccno1)sc1c(O)c(Cl)ccc12] |
| 53 | 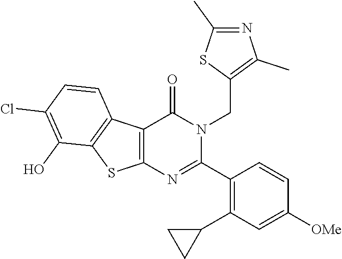 [SMILES: COc1ccc(-c2nc3sc4c(O)c(Cl)ccc4c3c(=O)n2Cc2sc(C)nc2C)c(C2CC2)c1] |
| 54 | 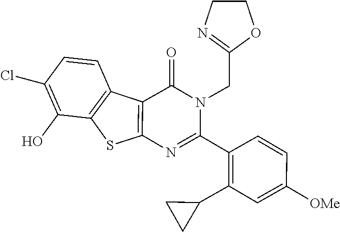 [SMILES: COc1ccc(-c2nc3sc4c(O)c(Cl)ccc4c3c(=O)n2CC2=NCCO2)c(C2CC2)c1] |
| 55 | 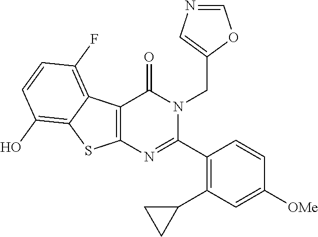 [SMILES: COc1ccc(-c2nc3sc4c(O)ccc(F)c4c3c(=O)n2Cc2cnco2)c(C2CC2)c1] |
| 56 | 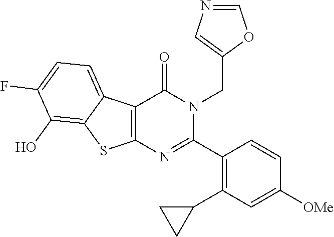 [SMILES: COc1ccc(-c2nc3sc4c(O)c(F)ccc4c3c(=O)n2Cc2cnco2)c(C2CC2)c1] |
| 57 | 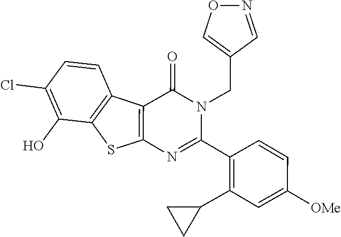 [SMILES: COc1ccc(-c2nc3sc4c(O)c(Cl)ccc4c3c(=O)n2Cc2cnoc2)c(C2CC2)c1] |
| 58 | 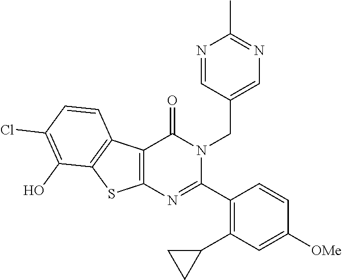 [SMILES: COc1ccc(-c2nc3sc4c(O)c(Cl)ccc4c3c(=O)n2Cc2cnc(C)nc2)c(C2CC2)c1] |
| 59 | 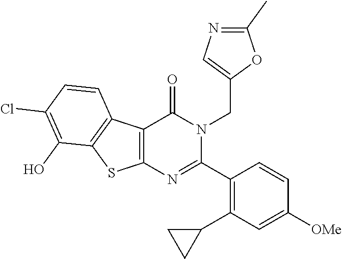 [SMILES: COc1ccc(-c2nc3sc4c(O)c(Cl)ccc4c3c(=O)n2Cc2cnc(C)o2)c(C2CC2)c1] |
| 60 | 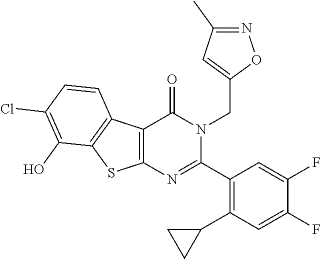 [SMILES: Cc1cc(Cn2c(-c3cc(F)c(F)cc3C3CC3)nc3sc4c(O)c(Cl)ccc4c3c2=O)on1] |
| 61 | 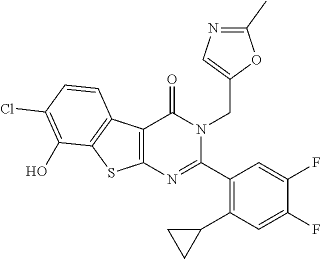 [SMILES: Cc1ncc(Cn2c(-c3cc(F)c(F)cc3C3CC3)nc3sc4c(O)c(Cl)ccc4c3c2=O)o1] |
| 62 | 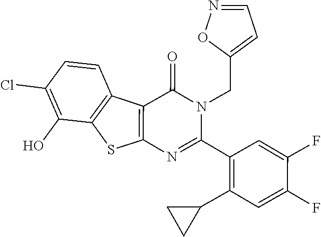 [SMILES: O=c1c2c(nc(-c3cc(F)c(F)cc3C3CC3)n1Cc1ccno1)sc1c(O)c(Cl)ccc12] |
| 63 | 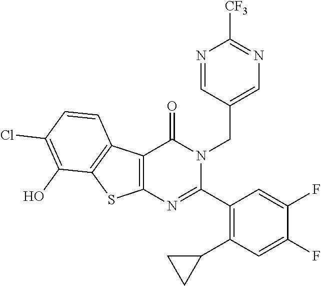 [SMILES: O=c1c2c(nc(-c3cc(F)c(F)cc3C3CC3)n1Cc1cnc(C(F)(F)F)nc1)sc1c(O)c(Cl)ccc12] |
| 64 | 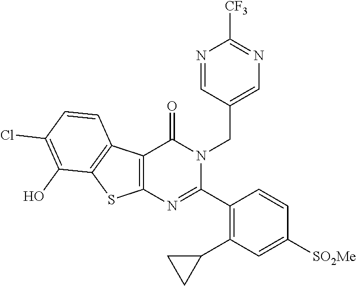 [SMILES: CS(=O)(=O)c1ccc(-c2nc3sc4c(O)c(Cl)ccc4c3c(=O)n2Cc2cnc(C(F)(F)F)nc2)c(C2CC2)c1] |
| 65 | 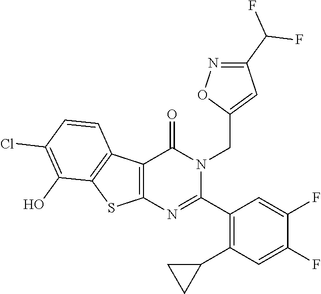 [SMILES: O=c1c2c(nc(-c3cc(F)c(F)cc3C3CC3)n1Cc1cc(C(F)F)no1)sc1c(O)c(Cl)ccc12] |
| 66 | 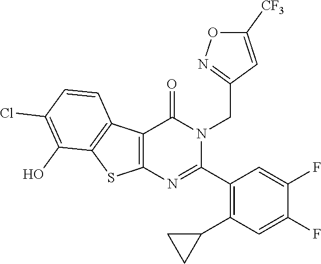 [SMILES: O=c1c2c(nc(-c3cc(F)c(F)cc3C3CC3)n1Cc1cc(C(F)(F)F)on1)sc1c(O)c(Cl)ccc12] |
| 67 | 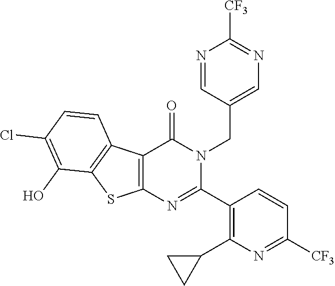 [SMILES: O=c1c2c(nc(-c3ccc(C(F)(F)F)nc3C3CC3)n1Cc1cnc(C(F)(F)F)nc1)sc1c(O)c(Cl)ccc12] |
| 68 | 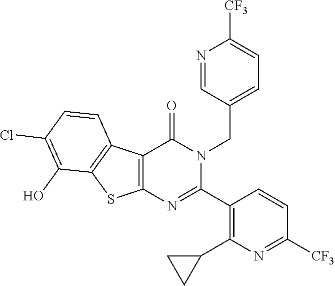 [SMILES: O=c1c2c(nc(-c3ccc(C(F)(F)F)nc3C3CC3)n1Cc1ccc(C(F)(F)F)nc1)sc1c(O)c(Cl)ccc12] |
| 69 | 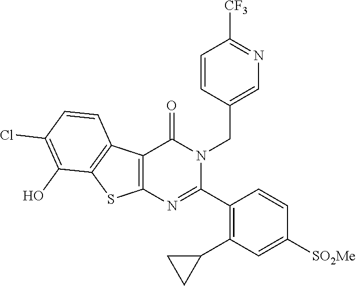 [SMILES: CS(=O)(=O)c1ccc(-c2nc3sc4c(O)c(Cl)ccc4c3c(=O)n2Cc2ccc(C(F)(F)F)nc2)c(C2CC2)c1] |
| 70 | 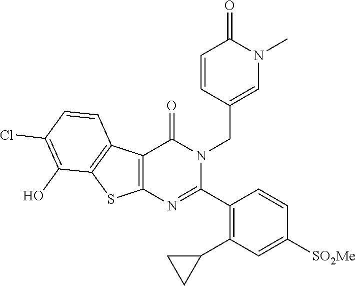 [SMILES: Cn1cc(Cn2c(-c3ccc(S(C)(=O)=O)cc3C3CC3)nc3sc4c(O)c(Cl)ccc4c3c2=O)ccc1=O] |
| 71 | 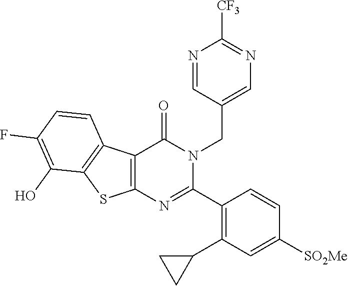 [SMILES: CS(=O)(=O)c1ccc(-c2nc3sc4c(O)c(F)ccc4c3c(=O)n2Cc2cnc(C(F)(F)F)nc2)c(C2CC2)c1] |
| 72 | 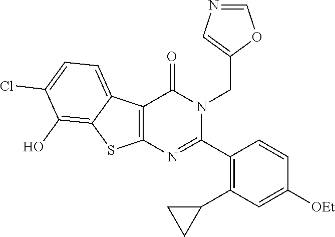 [SMILES: CCOc1ccc(-c2nc3sc4c(O)c(Cl)ccc4c3c(=O)n2Cc2cnco2)c(C2CC2)c1] |
| 73 | 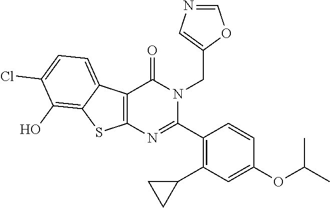 [SMILES: CC(C)Oc1ccc(-c2nc3sc4c(O)c(Cl)ccc4c3c(=O)n2Cc2cnco2)c(C2CC2)c1] |
| 74 | 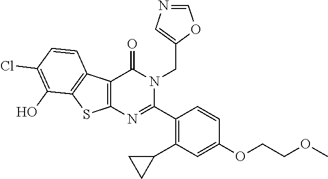 [SMILES: COCCOc1ccc(-c2nc3sc4c(O)c(Cl)ccc4c3c(=O)n2Cc2cnco2)c(C2CC2)c1] |
| 75 | 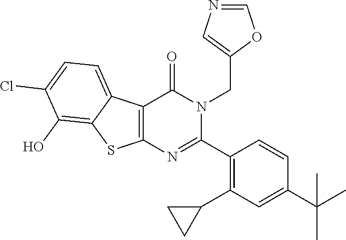 [SMILES: CC(C)(C)c1ccc(-c2nc3sc4c(O)c(Cl)ccc4c3c(=O)n2Cc2cnco2)c(C2CC2)c1] |
| 76 | 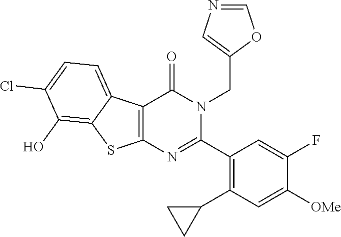 [SMILES: COc1cc(C2CC2)c(-c2nc3sc4c(O)c(Cl)ccc4c3c(=O)n2Cc2cnco2)cc1F] |
| 77 | 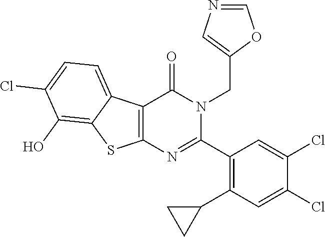 [SMILES: O=c1c2c(nc(-c3cc(Cl)c(Cl)cc3C3CC3)n1Cc1cnco1)sc1c(O)c(Cl)ccc12] |
| 78 | 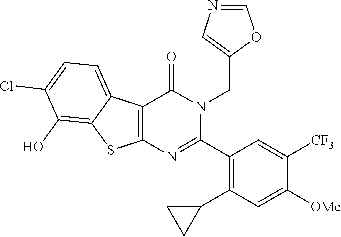 [SMILES: COc1cc(C2CC2)c(-c2nc3sc4c(O)c(Cl)ccc4c3c(=O)n2Cc2cnco2)cc1C(F)(F)F] |
| 79 | 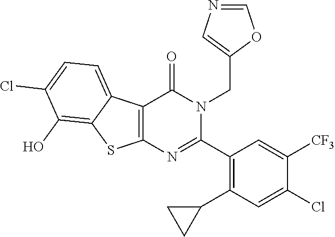 [SMILES: O=c1c2c(nc(-c3cc(C(F)(F)F)c(Cl)cc3C3CC3)n1Cc1cnco1)sc1c(O)c(Cl)ccc12] |
| 80 | 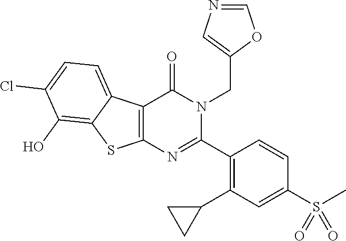 [SMILES: CS(=O)(=O)c1ccc(-c2nc3sc4c(O)c(Cl)ccc4c3c(=O)n2Cc2cnco2)c(C2CC2)c1] |
| 81 | 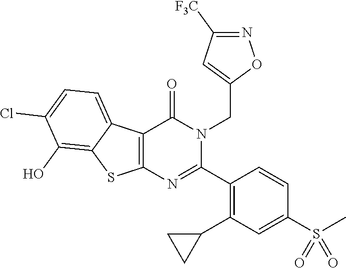 [SMILES: CS(=O)(=O)c1ccc(-c2nc3sc4c(O)c(Cl)ccc4c3c(=O)n2Cc2cc(C(F)(F)F)no2)c(C2CC2)c1] |
| 82 | 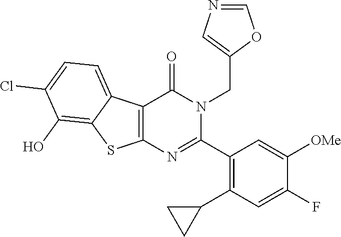 [SMILES: COc1cc(-c2nc3sc4c(O)c(Cl)ccc4c3c(=O)n2Cc2cnco2)c(C2CC2)cc1F] |
| 83 | 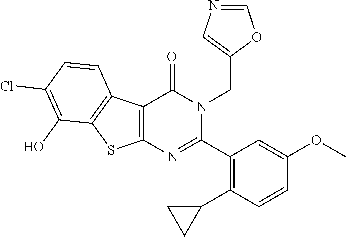 [SMILES: COc1ccc(C2CC2)c(-c2nc3sc4c(O)c(Cl)ccc4c3c(=O)n2Cc2cnco2)c1] |
| 84 | 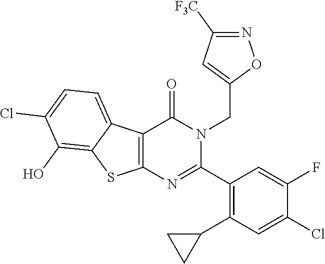 [SMILES: O=c1c2c(nc(-c3cc(F)c(Cl)cc3C3CC3)n1Cc1cc(C(F)(F)F)no1)sc1c(O)c(Cl)ccc12] |
| 85 | 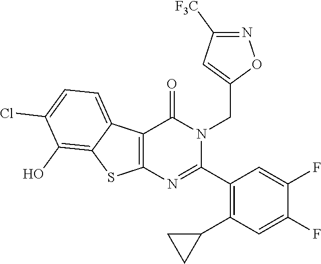 [SMILES: O=c1c2c(nc(-c3cc(F)c(F)cc3C3CC3)n1Cc1cc(C(F)(F)F)no1)sc1c(O)c(Cl)ccc12] |
| 86 | 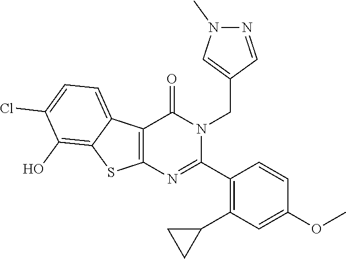 [SMILES: COc1ccc(-c2nc3sc4c(O)c(Cl)ccc4c3c(=O)n2Cc2cnn(C)c2)c(C2CC2)c1] |
| 87 | 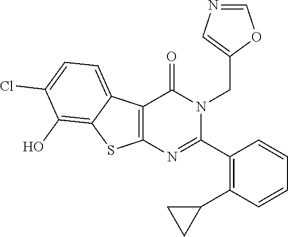 [SMILES: O=c1c2c(nc(-c3ccccc3C3CC3)n1Cc1cnco1)sc1c(O)c(Cl)ccc12] |
| 88 | 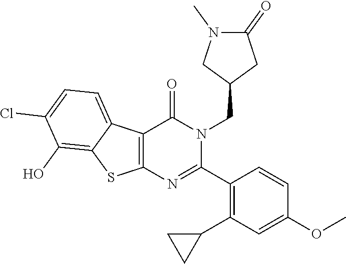 [SMILES: COc1ccc(-c2nc3sc4c(O)c(Cl)ccc4c3c(=O)n2C[C@@H]2CC(=O)N(C)C2)c(C2CC2)c1] |
| 89 | 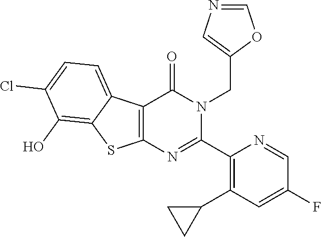 [SMILES: O=c1c2c(nc(-c3ncc(F)cc3C3CC3)n1Cc1cnco1)sc1c(O)c(Cl)ccc12] |
| 90 | 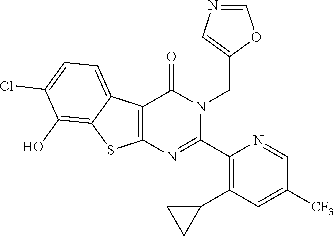 [SMILES: O=c1c2c(nc(-c3ncc(C(F)(F)F)cc3C3CC3)n1Cc1cnco1)sc1c(O)c(Cl)ccc12] |
| 91 | 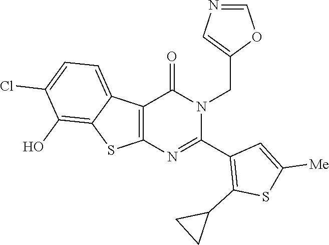 [SMILES: Cc1cc(-c2nc3sc4c(O)c(Cl)ccc4c3c(=O)n2Cc2cnco2)c(C2CC2)s1] |
| 92 | 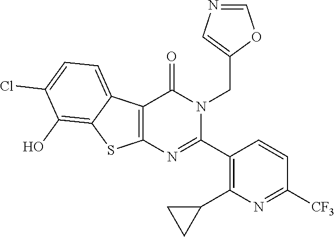 [SMILES: O=c1c2c(nc(-c3ccc(C(F)(F)F)nc3C3CC3)n1Cc1cnco1)sc1c(O)c(Cl)ccc12] |
| 93 | 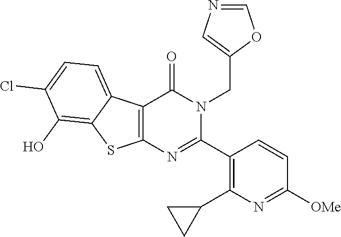 [SMILES: COc1ccc(-c2nc3sc4c(O)c(Cl)ccc4c3c(=O)n2Cc2cnco2)c(C2CC2)n1] |
| 94 | 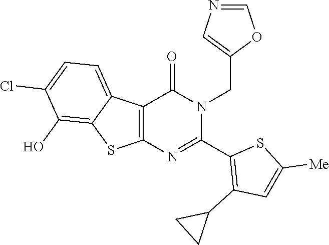 [SMILES: Cc1cc(C2CC2)c(-c2nc3sc4c(O)c(Cl)ccc4c3c(=O)n2Cc2cnco2)s1] |
| 95 | 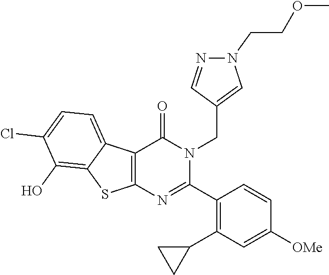 [SMILES: COCCn1cc(Cn2c(-c3ccc(OC)cc3C3CC3)nc3sc4c(O)c(Cl)ccc4c3c2=O)cn1] |
| 96 | 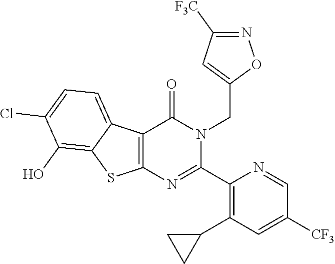 [SMILES: O=c1c2c(nc(-c3ncc(C(F)(F)F)cc3C3CC3)n1Cc1cc(C(F)(F)F)no1)sc1c(O)c(Cl)ccc12] |
| 97 | 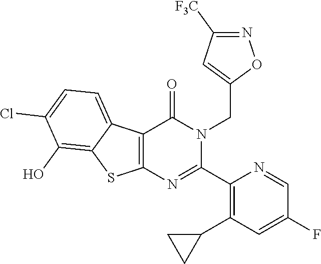 [SMILES: O=c1c2c(nc(-c3ncc(F)cc3C3CC3)n1Cc1cc(C(F)(F)F)no1)sc1c(O)c(Cl)ccc12] |
| 98 | 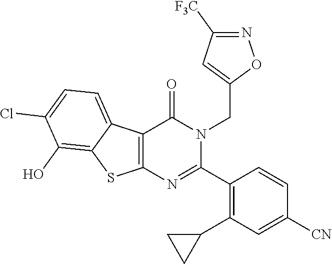 [SMILES: N#Cc1ccc(-c2nc3sc4c(O)c(Cl)ccc4c3c(=O)n2Cc2cc(C(F)(F)F)no2)c(C2CC2)c1] |
| 99 | 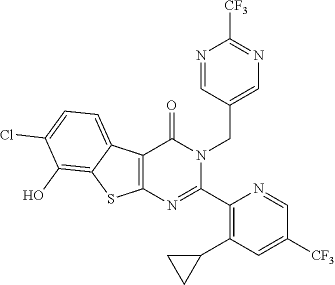 [SMILES: O=c1c2c(nc(-c3ncc(C(F)(F)F)cc3C3CC3)n1Cc1cnc(C(F)(F)F)nc1)sc1c(O)c(Cl)ccc12] |
| 100 | 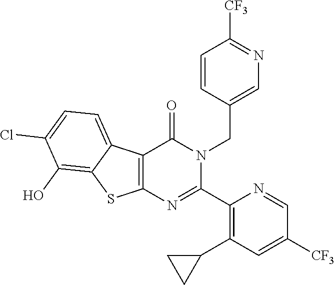 [SMILES: O=c1c2c(nc(-c3ncc(C(F)(F)F)cc3C3CC3)n1Cc1ccc(C(F)(F)F)nc1)sc1c(O)c(Cl)ccc12] |
| 101 | 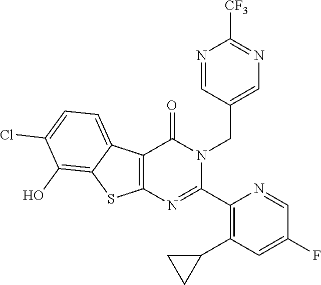 [SMILES: O=c1c2c(nc(-c3ncc(F)cc3C3CC3)n1Cc1cnc(C(F)(F)F)nc1)sc1c(O)c(Cl)ccc12] |
| 102 | 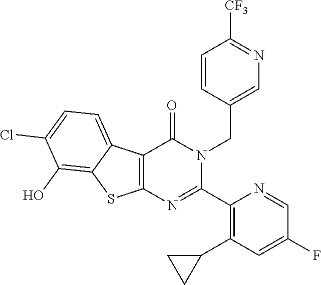 [SMILES: O=c1c2c(nc(-c3ncc(F)cc3C3CC3)n1Cc1ccc(C(F)(F)F)nc1)sc1c(O)c(Cl)ccc12] |
| 103 | 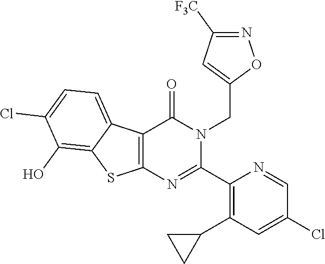 [SMILES: O=c1c2c(nc(-c3ncc(Cl)cc3C3CC3)n1Cc1cc(C(F)(F)F)no1)sc1c(O)c(Cl)ccc12] |
| 104 | 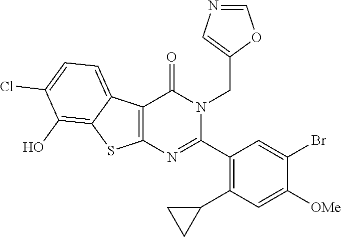 [SMILES: COc1cc(C2CC2)c(-c2nc3sc4c(O)c(Cl)ccc4c3c(=O)n2Cc2cnco2)cc1Br] |
| 105 | 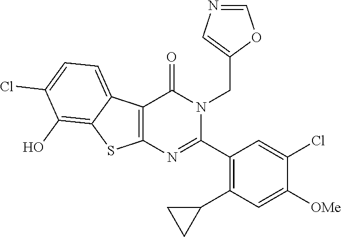 [SMILES: COc1cc(C2CC2)c(-c2nc3sc4c(O)c(Cl)ccc4c3c(=O)n2Cc2cnco2)cc1Cl] |
| 106 | 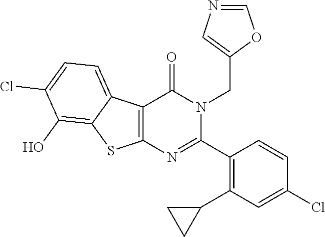 [SMILES: O=c1c2c(nc(-c3ccc(Cl)cc3C3CC3)n1Cc1cnco1)sc1c(O)c(Cl)ccc12] |
| 107 | 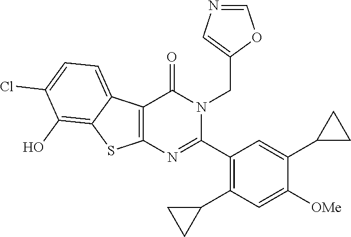 [SMILES: COc1cc(C2CC2)c(-c2nc3sc4c(O)c(Cl)ccc4c3c(=O)n2Cc2cnco2)cc1C1CC1] |
| 108 | 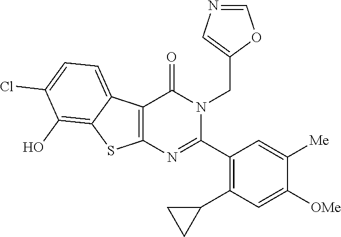 [SMILES: COc1cc(C2CC2)c(-c2nc3sc4c(O)c(Cl)ccc4c3c(=O)n2Cc2cnco2)cc1C] |
| 109 | 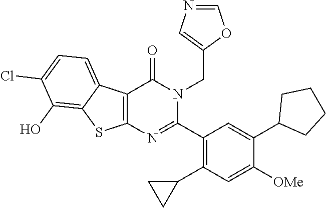 [SMILES: COc1cc(C2CC2)c(-c2nc3sc4c(O)c(Cl)ccc4c3c(=O)n2Cc2cnco2)cc1C1CCCC1] |
| 110 | 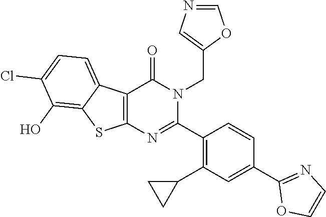 [SMILES: O=c1c2c(nc(-c3ccc(-c4ncco4)cc3C3CC3)n1Cc1cnco1)sc1c(O)c(Cl)ccc12] |
| 111 | 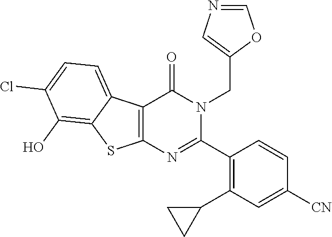 [SMILES: N#Cc1ccc(-c2nc3sc4c(O)c(Cl)ccc4c3c(=O)n2Cc2cnco2)c(C2CC2)c1] |
| 112 | 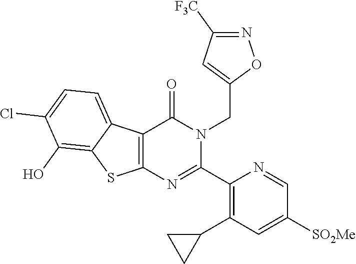 [SMILES: CS(=O)(=O)c1cnc(-c2nc3sc4c(O)c(Cl)ccc4c3c(=O)n2Cc2cc(C(F)(F)F)no2)c(C2CC2)c1] |
| 113 | 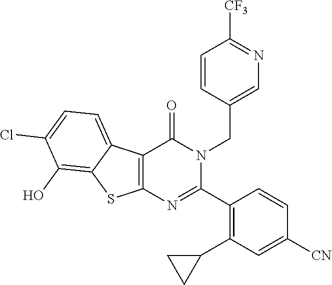 [SMILES: N#Cc1ccc(-c2nc3sc4c(O)c(Cl)ccc4c3c(=O)n2Cc2ccc(C(F)(F)F)nc2)c(C2CC2)c1] |
| 114 | 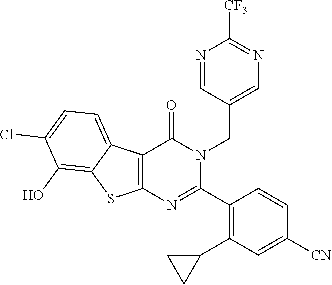 [SMILES: N#Cc1ccc(-c2nc3sc4c(O)c(Cl)ccc4c3c(=O)n2Cc2cnc(C(F)(F)F)nc2)c(C2CC2)c1] |
| 115 | 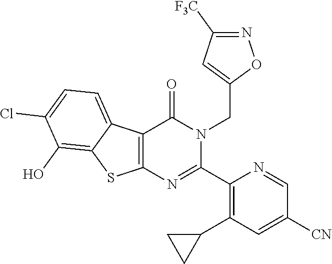 [SMILES: N#Cc1cnc(-c2nc3sc4c(O)c(Cl)ccc4c3c(=O)n2Cc2cc(C(F)(F)F)no2)c(C2CC2)c1] |
| 116 | 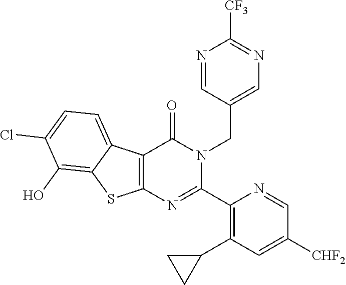 [SMILES: O=c1c2c(nc(-c3ncc(C(F)F)cc3C3CC3)n1Cc1cnc(C(F)(F)F)nc1)sc1c(O)c(Cl)ccc12] |
| 117 | 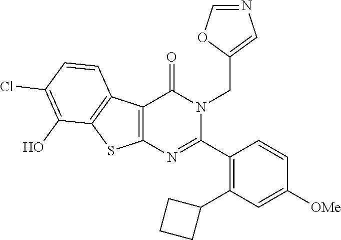 [SMILES: COc1ccc(-c2nc3sc4c(O)c(Cl)ccc4c3c(=O)n2Cc2cnco2)c(C2CCC2)c1] |
| 118 | 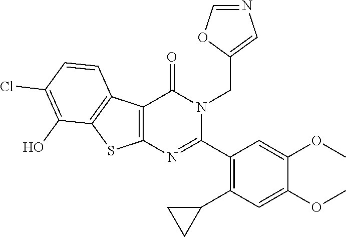 [SMILES: COc1cc(-c2nc3sc4c(O)c(Cl)ccc4c3c(=O)n2Cc2cnco2)c(C2CC2)cc1OC] |
| 119 | 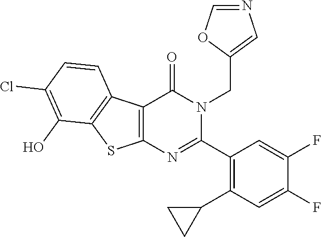 [SMILES: O=c1c2c(nc(-c3cc(F)c(F)cc3C3CC3)n1Cc1cnco1)sc1c(O)c(Cl)ccc12] |
| 120 | 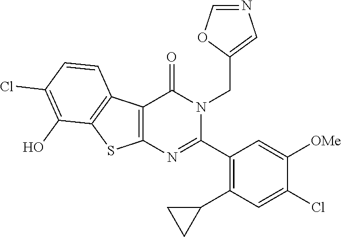 [SMILES: COc1cc(-c2nc3sc4c(O)c(Cl)ccc4c3c(=O)n2Cc2cnco2)c(C2CC2)cc1Cl] |
| 121 | 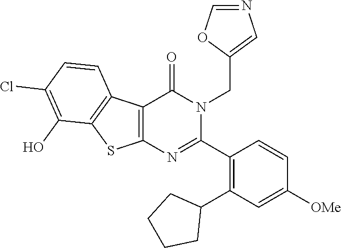 [SMILES: COc1ccc(-c2nc3sc4c(O)c(Cl)ccc4c3c(=O)n2Cc2cnco2)c(C2CCCC2)c1] |
| 122 | 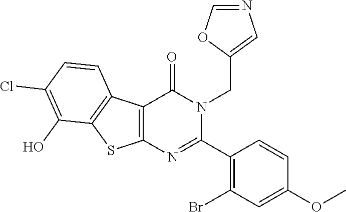 [SMILES: COc1ccc(-c2nc3sc4c(O)c(Cl)ccc4c3c(=O)n2Cc2cnco2)c(Br)c1] |
| 123 | 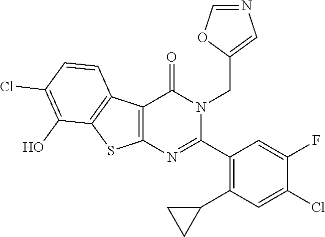 [SMILES: O=c1c2c(nc(-c3cc(F)c(Cl)cc3C3CC3)n1Cc1cnco1)sc1c(O)c(Cl)ccc12] |
| 124 | 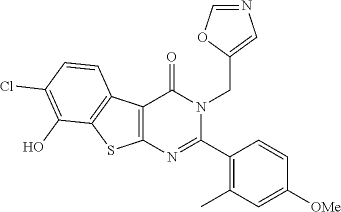 [SMILES: COc1ccc(-c2nc3sc4c(O)c(Cl)ccc4c3c(=O)n2Cc2cnco2)c(C)c1] |
| 125 | 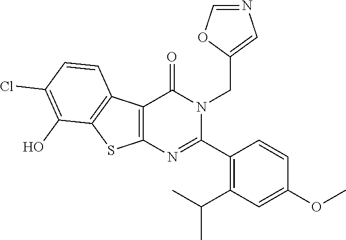 [SMILES: COc1ccc(-c2nc3sc4c(O)c(Cl)ccc4c3c(=O)n2Cc2cnco2)c(C(C)C)c1] |
| 126 | 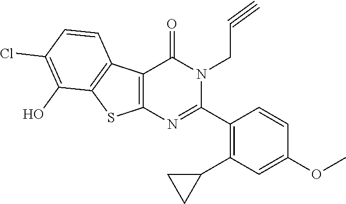 [SMILES: C#CCn1c(-c2ccc(OC)cc2C2CC2)nc2sc3c(O)c(Cl)ccc3c2c1=O] |
| 127 | 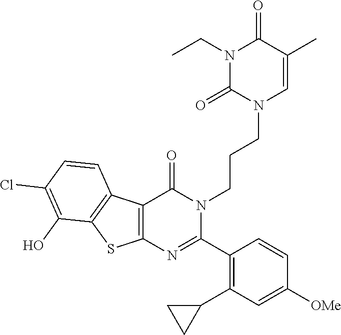 [SMILES: CCn1c(=O)c(C)cn(CCCn2c(-c3ccc(OC)cc3C3CC3)nc3sc4c(O)c(Cl)ccc4c3c2=O)c1=O] |
| 128 | 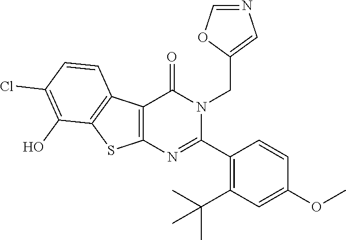 [SMILES: COc1ccc(-c2nc3sc4c(O)c(Cl)ccc4c3c(=O)n2Cc2cnco2)c(C(C)(C)C)c1] |
| 129 | 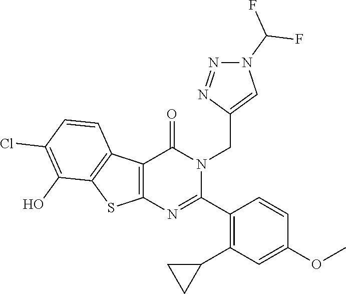 [SMILES: COc1ccc(-c2nc3sc4c(O)c(Cl)ccc4c3c(=O)n2Cc2cn(C(F)F)nn2)c(C2CC2)c1] |
| 130 | 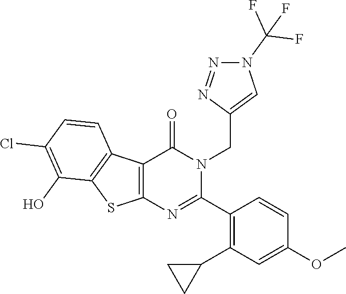 [SMILES: COc1ccc(-c2nc3sc4c(O)c(Cl)ccc4c3c(=O)n2Cc2cn(C(F)(F)F)nn2)c(C2CC2)c1] |
| 131 | 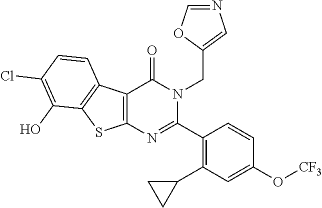 [SMILES: O=c1c2c(nc(-c3ccc(OC(F)(F)F)cc3C3CC3)n1Cc1cnco1)sc1c(O)c(Cl)ccc12] |
| 132 | 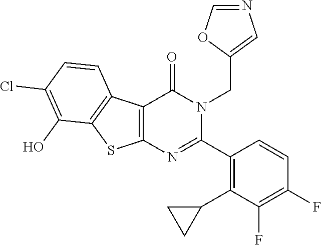 [SMILES: O=c1c2c(nc(-c3ccc(F)c(F)c3C3CC3)n1Cc1cnco1)sc1c(O)c(Cl)ccc12] |
| 133 | 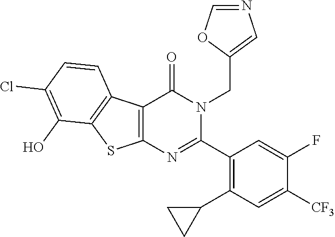 [SMILES: O=c1c2c(nc(-c3cc(F)c(C(F)(F)F)cc3C3CC3)n1Cc1cnco1)sc1c(O)c(Cl)ccc12] |
| 134 | 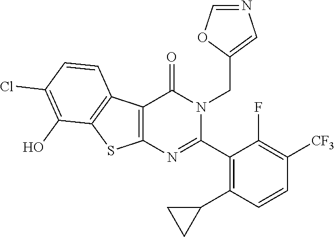 [SMILES: O=c1c2c(nc(-c3c(C4CC4)ccc(C(F)(F)F)c3F)n1Cc1cnco1)sc1c(O)c(Cl)ccc12] |
| 135 | 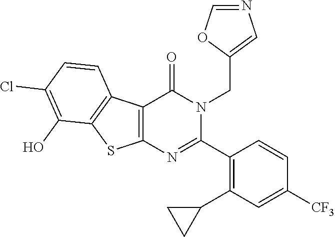 [SMILES: O=c1c2c(nc(-c3ccc(C(F)(F)F)cc3C3CC3)n1Cc1cnco1)sc1c(O)c(Cl)ccc12] |
| 136 | 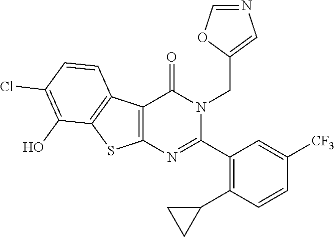 [SMILES: O=c1c2c(nc(-c3cc(C(F)(F)F)ccc3C3CC3)n1Cc1cnco1)sc1c(O)c(Cl)ccc12] |
| 137 | 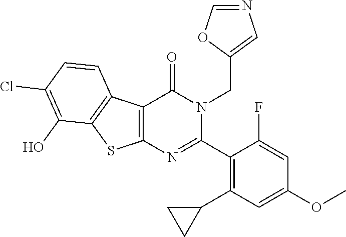 [SMILES: COc1cc(F)c(-c2nc3sc4c(O)c(Cl)ccc4c3c(=O)n2Cc2cnco2)c(C2CC2)c1] |
| 138 | 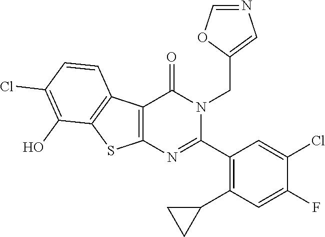 [SMILES: O=c1c2c(nc(-c3cc(Cl)c(F)cc3C3CC3)n1Cc1cnco1)sc1c(O)c(Cl)ccc12] |
| 139 | 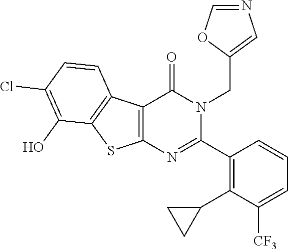 [SMILES: O=c1c2c(nc(-c3cccc(C(F)(F)F)c3C3CC3)n1Cc1cnco1)sc1c(O)c(Cl)ccc12] |
| 140 | 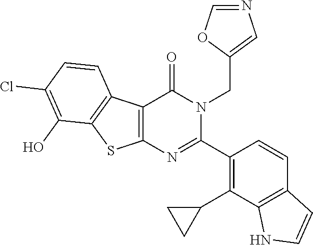 [SMILES: O=c1c2c(nc(-c3ccc4cc[nH]c4c3C3CC3)n1Cc1cnco1)sc1c(O)c(Cl)ccc12] |
| 141 | 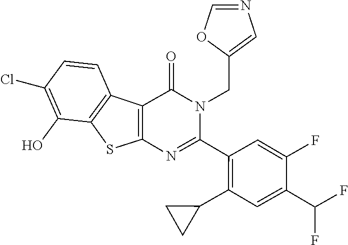 [SMILES: O=c1c2c(nc(-c3cc(F)c(C(F)F)cc3C3CC3)n1Cc1cnco1)sc1c(O)c(Cl)ccc12] |
| 142 | 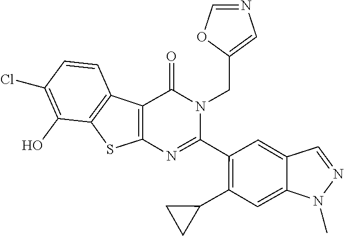 [SMILES: Cn1ncc2cc(-c3nc4sc5c(O)c(Cl)ccc5c4c(=O)n3Cc3cnco3)c(C3CC3)cc21] |
| 143 | 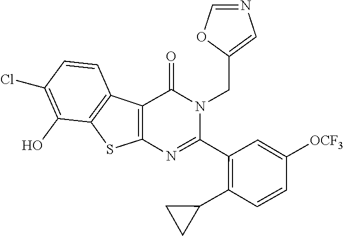 [SMILES: O=c1c2c(nc(-c3cc(OC(F)(F)F)ccc3C3CC3)n1Cc1cnco1)sc1c(O)c(Cl)ccc12] |
| 144 | 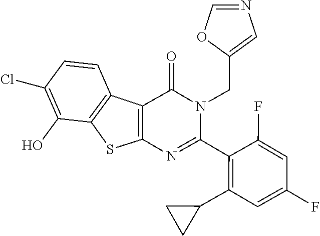 [SMILES: O=c1c2c(nc(-c3c(F)cc(F)cc3C3CC3)n1Cc1cnco1)sc1c(O)c(Cl)ccc12] |
| 145 | 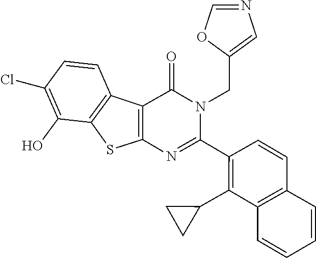 [SMILES: O=c1c2c(nc(-c3ccc4ccccc4c3C3CC3)n1Cc1cnco1)sc1c(O)c(Cl)ccc12] |
| 146 | 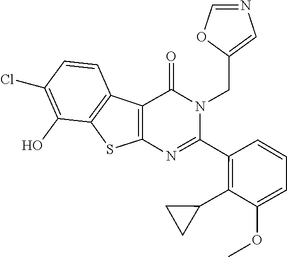 [SMILES: COc1cccc(-c2nc3sc4c(O)c(Cl)ccc4c3c(=O)n2Cc2cnco2)c1C1CC1] |
| 147 | 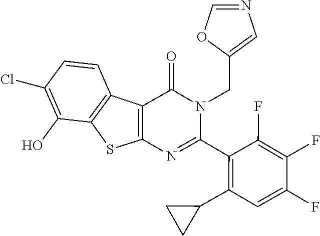 [SMILES: O=c1c2c(nc(-c3c(C4CC4)cc(F)c(F)c3F)n1Cc1cnco1)sc1c(O)c(Cl)ccc12] |
| 148 | 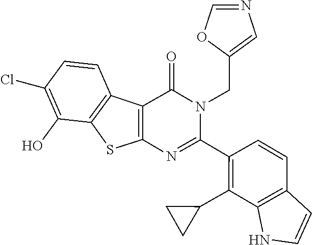 [SMILES: O=c1c2c(nc(-c3ccc4cc[nH]c4c3C3CC3)n1Cc1cnco1)sc1c(O)c(Cl)ccc12] |
| 149 | 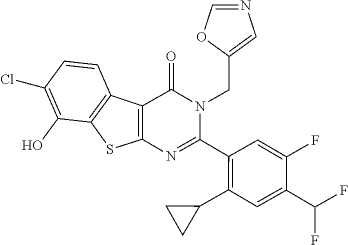 [SMILES: O=c1c2c(nc(-c3cc(F)c(C(F)F)cc3C3CC3)n1Cc1cnco1)sc1c(O)c(Cl)ccc12] |
| 150 | 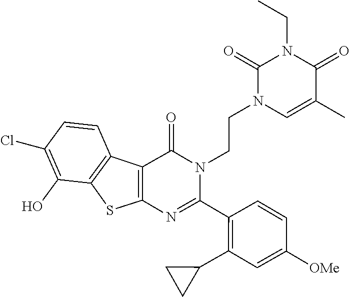 [SMILES: CCn1c(=O)c(C)cn(CCn2c(-c3ccc(OC)cc3C3CC3)nc3sc4c(O)c(Cl)ccc4c3c2=O)c1=O] |
| 151 | 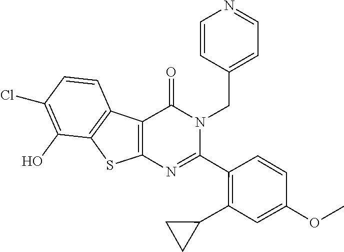 [SMILES: COc1ccc(-c2nc3sc4c(O)c(Cl)ccc4c3c(=O)n2Cc2ccncc2)c(C2CC2)c1] |
| 152 | 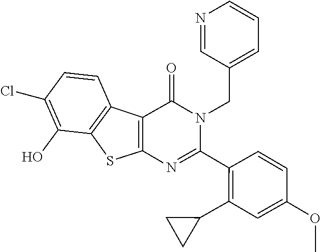 [SMILES: COc1ccc(-c2nc3sc4c(O)c(Cl)ccc4c3c(=O)n2Cc2cccnc2)c(C2CC2)c1] |
| 153 | 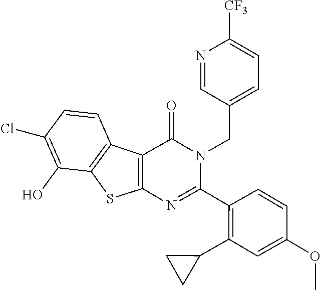 [SMILES: COc1ccc(-c2nc3sc4c(O)c(Cl)ccc4c3c(=O)n2Cc2ccc(C(F)(F)F)nc2)c(C2CC2)c1] |
| 154 | 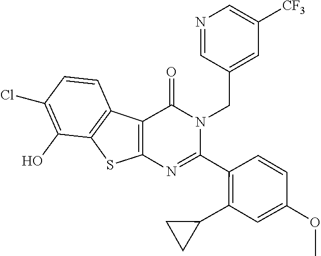 [SMILES: COc1ccc(-c2nc3sc4c(O)c(Cl)ccc4c3c(=O)n2Cc2cncc(C(F)(F)F)c2)c(C2CC2)c1] |
| 155 | 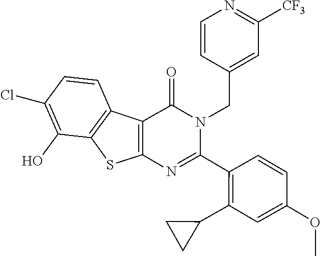 [SMILES: COc1ccc(-c2nc3sc4c(O)c(Cl)ccc4c3c(=O)n2Cc2ccnc(C(F)(F)F)c2)c(C2CC2)c1] |
| 156 | 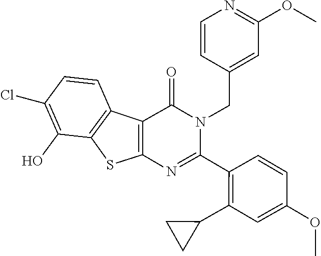 [SMILES: COc1ccc(-c2nc3sc4c(O)c(Cl)ccc4c3c(=O)n2Cc2ccnc(OC)c2)c(C2CC2)c1] |
| 157 | 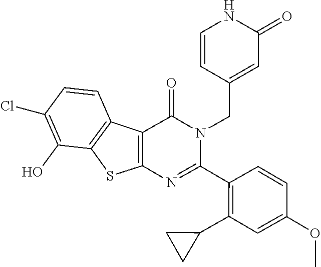 [SMILES: COc1ccc(-c2nc3sc4c(O)c(Cl)ccc4c3c(=O)n2Cc2cc[nH]c(=O)c2)c(C2CC2)c1] |
| 158 | 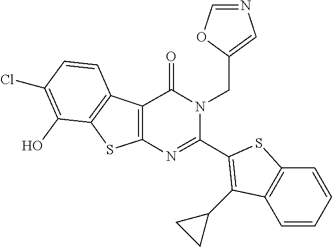 [SMILES: O=c1c2c(nc(-c3sc4ccccc4c3C3CC3)n1Cc1cnco1)sc1c(O)c(Cl)ccc12] |
| 159 | 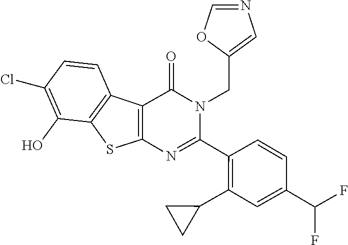 [SMILES: O=c1c2c(nc(-c3ccc(C(F)F)cc3C3CC3)n1Cc1cnco1)sc1c(O)c(Cl)ccc12] |
| 160 | 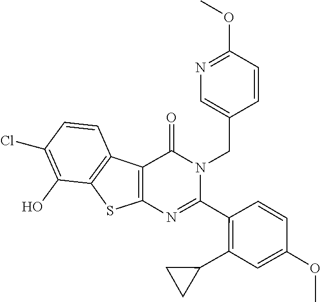 [SMILES: COc1ccc(-c2nc3sc4c(O)c(Cl)ccc4c3c(=O)n2Cc2ccc(OC)nc2)c(C2CC2)c1] |
| 161 | 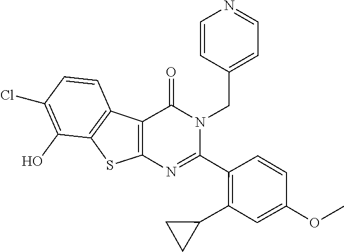 [SMILES: COc1ccc(-c2nc3sc4c(O)c(Cl)ccc4c3c(=O)n2Cc2ccncc2)c(C2CC2)c1] |
| 162 | 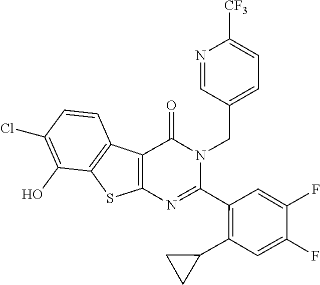 [SMILES: O=c1c2c(nc(-c3cc(F)c(F)cc3C3CC3)n1Cc1ccc(C(F)(F)F)nc1)sc1c(O)c(Cl)ccc12] |
| 163 | 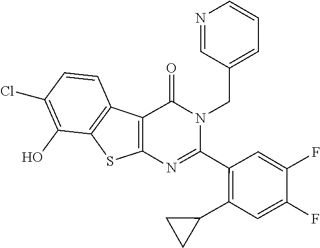 [SMILES: O=c1c2c(nc(-c3cc(F)c(F)cc3C3CC3)n1Cc1cccnc1)sc1c(O)c(Cl)ccc12] |
| 164 | 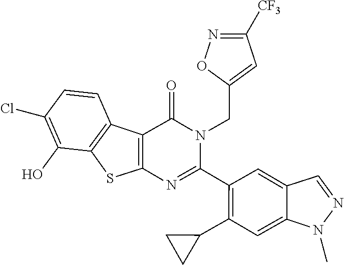 [SMILES: Cn1ncc2cc(-c3nc4sc5c(O)c(Cl)ccc5c4c(=O)n3Cc3cc(C(F)(F)F)no3)c(C3CC3)cc21] |
| 165 | 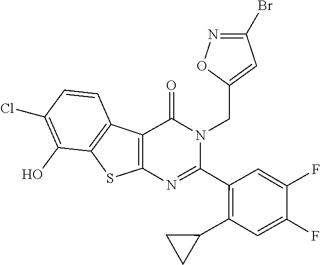 [SMILES: O=c1c2c(nc(-c3cc(F)c(F)cc3C3CC3)n1Cc1cc(Br)no1)sc1c(O)c(Cl)ccc12] |
| 166 | 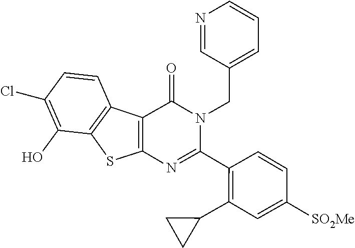 [SMILES: CS(=O)(=O)c1ccc(-c2nc3sc4c(O)c(Cl)ccc4c3c(=O)n2Cc2cccnc2)c(C2CC2)c1] |
| 167 | 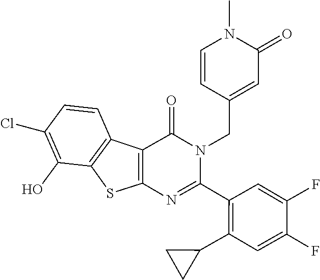 [SMILES: Cn1ccc(Cn2c(-c3cc(F)c(F)cc3C3CC3)nc3sc4c(O)c(Cl)ccc4c3c2=O)cc1=O] |
| 168 | 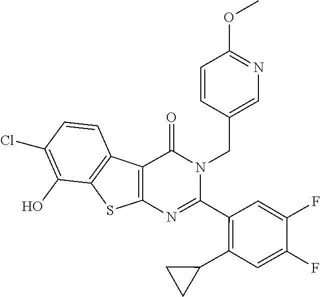 [SMILES: COc1ccc(Cn2c(-c3cc(F)c(F)cc3C3CC3)nc3sc4c(O)c(Cl)ccc4c3c2=O)cn1] |
| 169 | 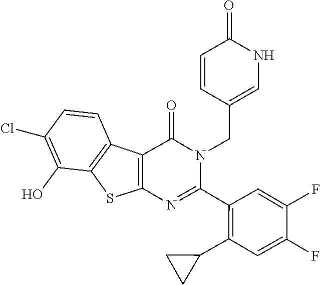 [SMILES: O=c1ccc(Cn2c(-c3cc(F)c(F)cc3C3CC3)nc3sc4c(O)c(Cl)ccc4c3c2=O)c[nH]1] |
| 170 | 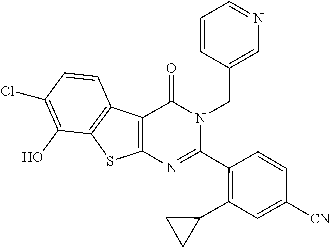 [SMILES: N#Cc1ccc(-c2nc3sc4c(O)c(Cl)ccc4c3c(=O)n2Cc2cccnc2)c(C2CC2)c1] |
| 171 | 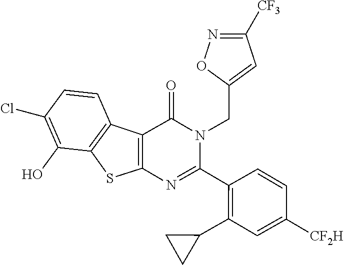 [SMILES: O=c1c2c(nc(-c3ccc(C(F)F)cc3C3CC3)n1Cc1cc(C(F)(F)F)no1)sc1c(O)c(Cl)ccc12] |
| 172 | 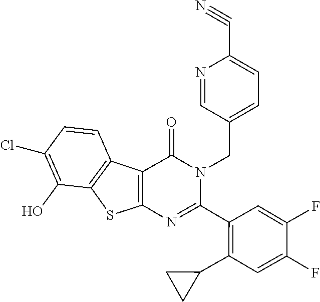 [SMILES: N#Cc1ccc(Cn2c(-c3cc(F)c(F)cc3C3CC3)nc3sc4c(O)c(Cl)ccc4c3c2=O)cn1] |
| 173 | 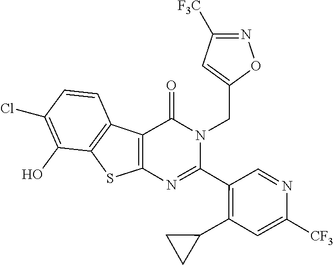 [SMILES: O=c1c2c(nc(-c3cnc(C(F)(F)F)cc3C3CC3)n1Cc1cc(C(F)(F)F)no1)sc1c(O)c(Cl)ccc12] |
| 174 | 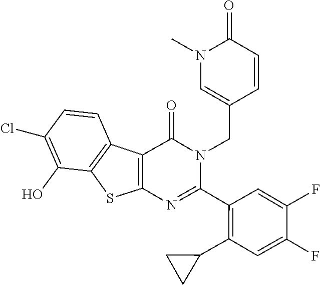 [SMILES: Cn1cc(Cn2c(-c3cc(F)c(F)cc3C3CC3)nc3sc4c(O)c(Cl)ccc4c3c2=O)ccc1=O] |
| 175 | 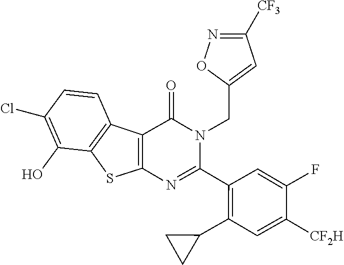 [SMILES: O=c1c2c(nc(-c3cc(F)c(C(F)F)cc3C3CC3)n1Cc1cc(C(F)(F)F)no1)sc1c(O)c(Cl)ccc12] |
| 176 | 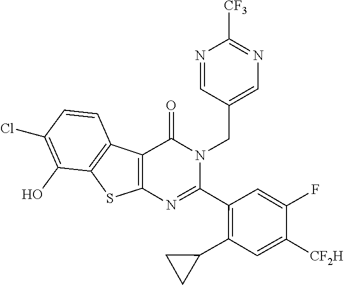 [SMILES: O=c1c2c(nc(-c3cc(F)c(C(F)F)cc3C3CC3)n1Cc1cnc(C(F)(F)F)nc1)sc1c(O)c(Cl)ccc12] |
| 177 | 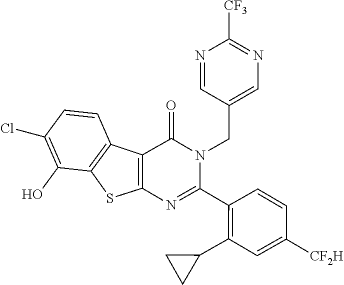 [SMILES: O=c1c2c(nc(-c3ccc(C(F)F)cc3C3CC3)n1Cc1cnc(C(F)(F)F)nc1)sc1c(O)c(Cl)ccc12] |
| 178 | 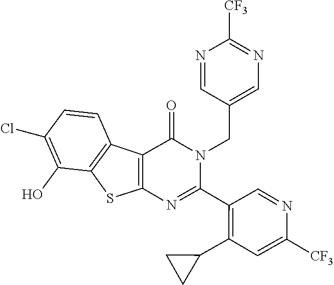 [SMILES: O=c1c2c(nc(-c3cnc(C(F)(F)F)cc3C3CC3)n1Cc1cnc(C(F)(F)F)nc1)sc1c(O)c(Cl)ccc12] |
| 179 | 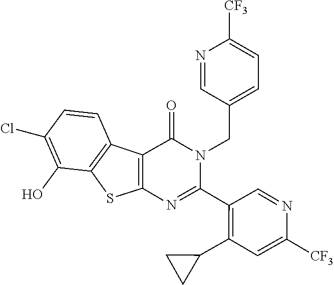 [SMILES: O=c1c2c(nc(-c3cnc(C(F)(F)F)cc3C3CC3)n1Cc1ccc(C(F)(F)F)nc1)sc1c(O)c(Cl)ccc12] |
| 180 | 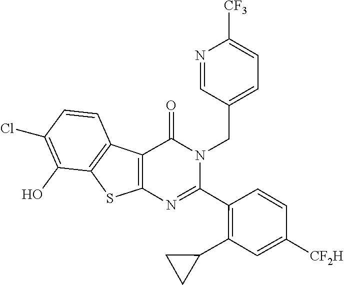 [SMILES: O=c1c2c(nc(-c3ccc(C(F)F)cc3C3CC3)n1Cc1ccc(C(F)(F)F)nc1)sc1c(O)c(Cl)ccc12] |
| 181 | 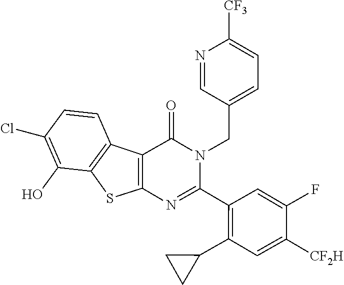 [SMILES: O=c1c2c(nc(-c3cc(F)c(C(F)F)cc3C3CC3)n1Cc1ccc(C(F)(F)F)nc1)sc1c(O)c(Cl)ccc12] |
| 182 | 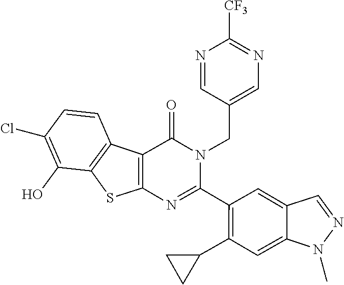 [SMILES: Cn1ncc2cc(-c3nc4sc5c(O)c(Cl)ccc5c4c(=O)n3Cc3cnc(C(F)(F)F)nc3)c(C3CC3)cc21] |
| 183 | 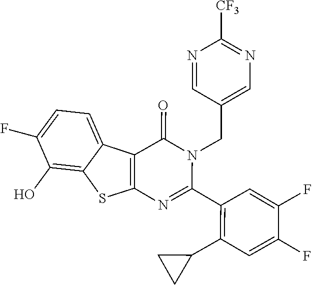 [SMILES: O=c1c2c(nc(-c3cc(F)c(F)cc3C3CC3)n1Cc1cnc(C(F)(F)F)nc1)sc1c(O)c(F)ccc12] |
| 184 | 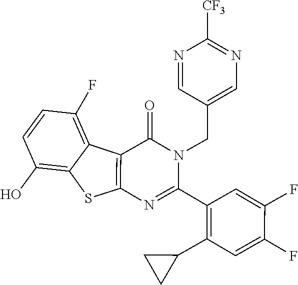 [SMILES: O=c1c2c(nc(-c3cc(F)c(F)cc3C3CC3)n1Cc1cnc(C(F)(F)F)nc1)sc1c(O)ccc(F)c12] |
| 185 | 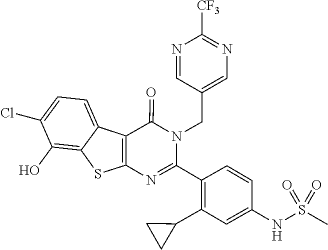 [SMILES: CS(=O)(=O)Nc1ccc(-c2nc3sc4c(O)c(Cl)ccc4c3c(=O)n2Cc2cnc(C(F)(F)F)nc2)c(C2CC2)c1] |
| 186 | 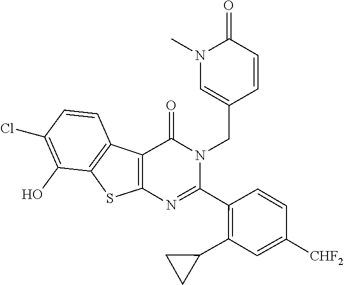 [SMILES: Cn1cc(Cn2c(-c3ccc(C(F)F)cc3C3CC3)nc3sc4c(O)c(Cl)ccc4c3c2=O)ccc1=O] |
| 187 | 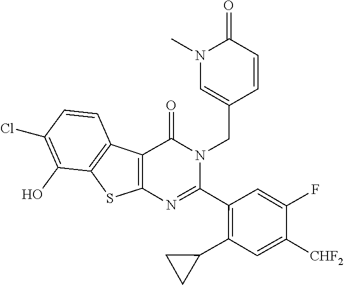 [SMILES: Cn1cc(Cn2c(-c3cc(F)c(C(F)F)cc3C3CC3)nc3sc4c(O)c(Cl)ccc4c3c2=O)ccc1=O] |
| 188 | 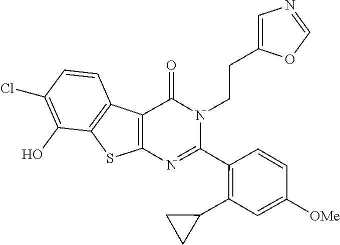 [SMILES: COc1ccc(-c2nc3sc4c(O)c(Cl)ccc4c3c(=O)n2CCc2cnco2)c(C2CC2)c1] |
| 189 | 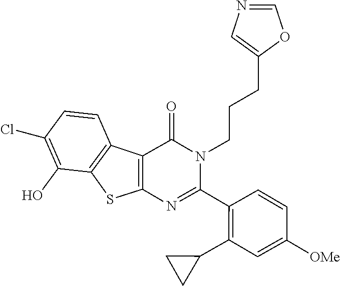 [SMILES: COc1ccc(-c2nc3sc4c(O)c(Cl)ccc4c3c(=O)n2CCCc2cnco2)c(C2CC2)c1] |
| 190 | 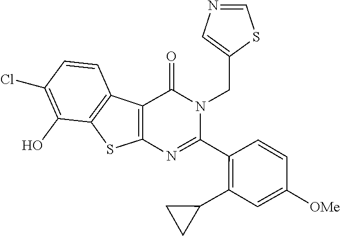 [SMILES: COc1ccc(-c2nc3sc4c(O)c(Cl)ccc4c3c(=O)n2Cc2cncs2)c(C2CC2)c1] |
| 191 | 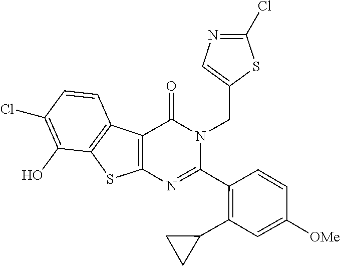 [SMILES: COc1ccc(-c2nc3sc4c(O)c(Cl)ccc4c3c(=O)n2Cc2cnc(Cl)s2)c(C2CC2)c1] |
| 192 | 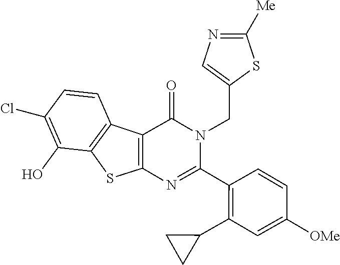 [SMILES: COc1ccc(-c2nc3sc4c(O)c(Cl)ccc4c3c(=O)n2Cc2cnc(C)s2)c(C2CC2)c1] |
| 193 | 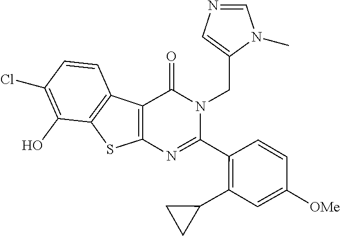 [SMILES: COc1ccc(-c2nc3sc4c(O)c(Cl)ccc4c3c(=O)n2Cc2cncn2C)c(C2CC2)c1] |
| 194 | 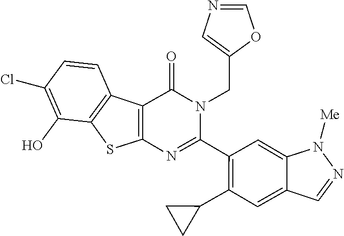 [SMILES: Cn1ncc2cc(C3CC3)c(-c3nc4sc5c(O)c(Cl)ccc5c4c(=O)n3Cc3cnco3)cc21] |
| 195 | 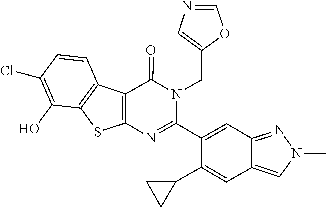 [SMILES: Cn1cc2cc(C3CC3)c(-c3nc4sc5c(O)c(Cl)ccc5c4c(=O)n3Cc3cnco3)cc2n1] |
| 196 | 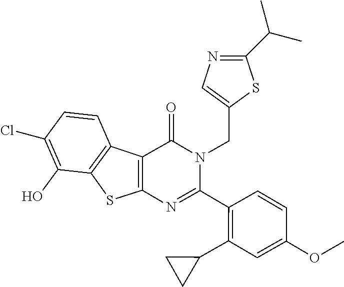 [SMILES: COc1ccc(-c2nc3sc4c(O)c(Cl)ccc4c3c(=O)n2Cc2cnc(C(C)C)s2)c(C2CC2)c1] |
| 197 | 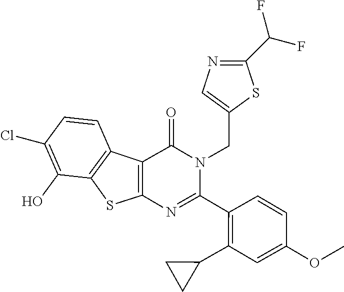 [SMILES: COc1ccc(-c2nc3sc4c(O)c(Cl)ccc4c3c(=O)n2Cc2cnc(C(F)F)s2)c(C2CC2)c1] |
| 198 | 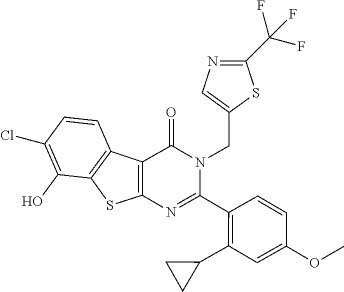 [SMILES: COc1ccc(-c2nc3sc4c(O)c(Cl)ccc4c3c(=O)n2Cc2cnc(C(F)(F)F)s2)c(C2CC2)c1] |
| 199 | 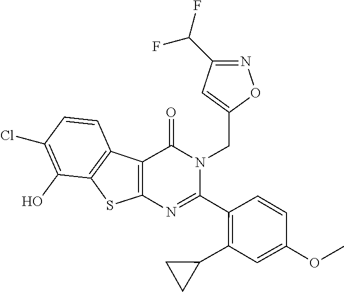 [SMILES: COc1ccc(-c2nc3sc4c(O)c(Cl)ccc4c3c(=O)n2Cc2cc(C(F)F)no2)c(C2CC2)c1] |
| 200 | 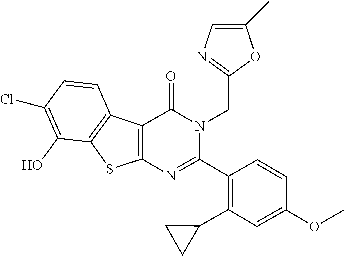 [SMILES: COc1ccc(-c2nc3sc4c(O)c(Cl)ccc4c3c(=O)n2Cc2ncc(C)o2)c(C2CC2)c1] |
| 201 | 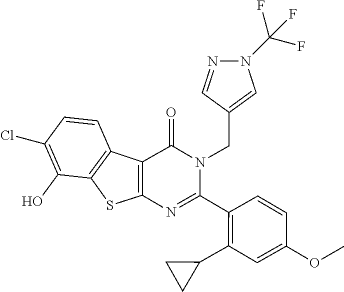 [SMILES: COc1ccc(-c2nc3sc4c(O)c(Cl)ccc4c3c(=O)n2Cc2cnn(C(F)(F)F)c2)c(C2CC2)c1] |
| 202 | 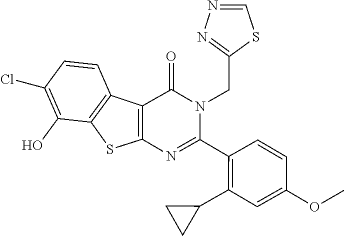 [SMILES: COc1ccc(-c2nc3sc4c(O)c(Cl)ccc4c3c(=O)n2Cc2nncs2)c(C2CC2)c1] |
| 203 | 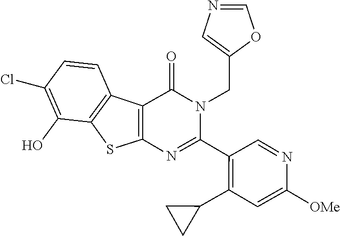 [SMILES: COc1cc(C2CC2)c(-c2nc3sc4c(O)c(Cl)ccc4c3c(=O)n2Cc2cnco2)cn1] |
| 204 | 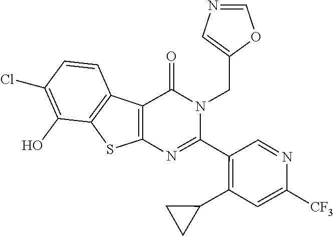 [SMILES: O=c1c2c(nc(-c3cnc(C(F)(F)F)cc3C3CC3)n1Cc1cnco1)sc1c(O)c(Cl)ccc12] |
| 205 | 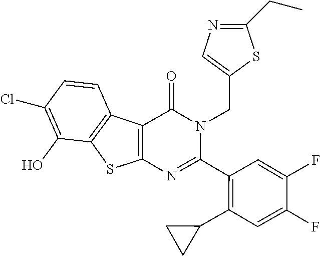 [SMILES: CCc1ncc(Cn2c(-c3cc(F)c(F)cc3C3CC3)nc3sc4c(O)c(Cl)ccc4c3c2=O)s1] |
| 206 | 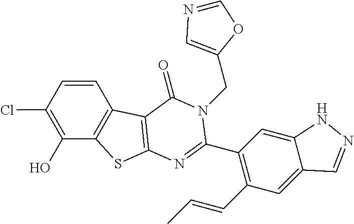 [SMILES: C/C=C/c1cc2cn[nH]c2cc1-c1nc2sc3c(O)c(Cl)ccc3c2c(=O)n1Cc1cnco1] |
| 207 | 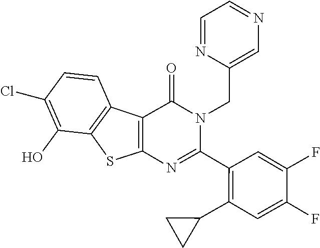 [SMILES: O=c1c2c(nc(-c3cc(F)c(F)cc3C3CC3)n1Cc1cnccn1)sc1c(O)c(Cl)ccc12] |
| 208 | 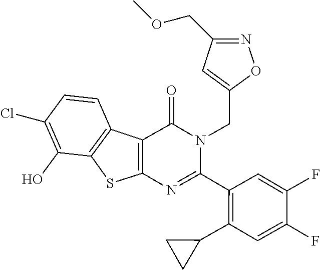 [SMILES: COCc1cc(Cn2c(-c3cc(F)c(F)cc3C3CC3)nc3sc4c(O)c(Cl)ccc4c3c2=O)on1] |
| 209 | 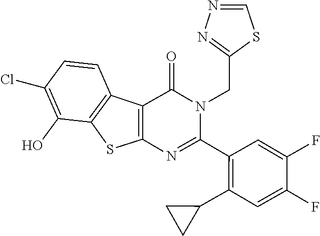 [SMILES: O=c1c2c(nc(-c3cc(F)c(F)cc3C3CC3)n1Cc1nncs1)sc1c(O)c(Cl)ccc12] |
| 210 | 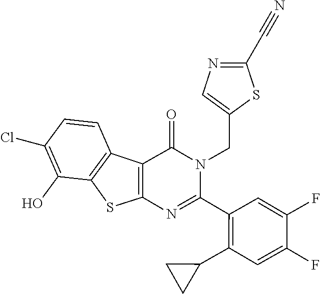 [SMILES: N#Cc1ncc(Cn2c(-c3cc(F)c(F)cc3C3CC3)nc3sc4c(O)c(Cl)ccc4c3c2=O)s1] |
| 211 | 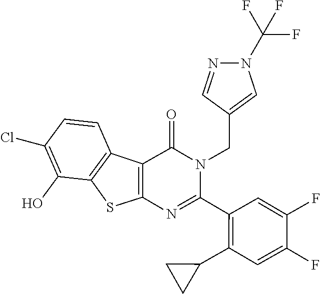 [SMILES: O=c1c2c(nc(-c3cc(F)c(F)cc3C3CC3)n1Cc1cnn(C(F)(F)F)c1)sc1c(O)c(Cl)ccc12] |
| 212 | 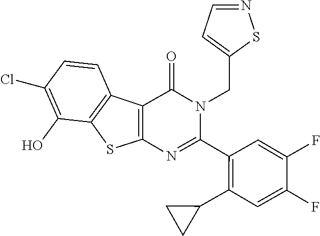 [SMILES: O=c1c2c(nc(-c3cc(F)c(F)cc3C3CC3)n1Cc1ccns1)sc1c(O)c(Cl)ccc12] |
| 213 | 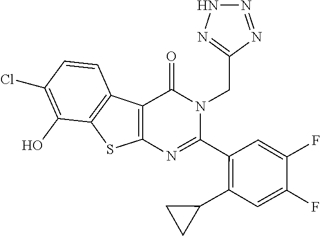 [SMILES: O=c1c2c(nc(-c3cc(F)c(F)cc3C3CC3)n1Cc1nn[nH]n1)sc1c(O)c(Cl)ccc12] |
| 214 | 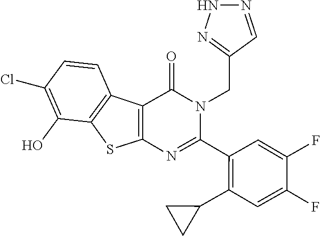 [SMILES: O=c1c2c(nc(-c3cc(F)c(F)cc3C3CC3)n1Cc1cn[nH]n1)sc1c(O)c(Cl)ccc12] |
| 215 | 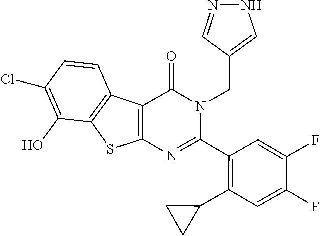 [SMILES: O=c1c2c(nc(-c3cc(F)c(F)cc3C3CC3)n1Cc1cn[nH]c1)sc1c(O)c(Cl)ccc12] |
| 216 | 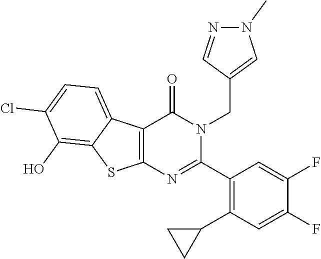 [SMILES: Cn1cc(Cn2c(-c3cc(F)c(F)cc3C3CC3)nc3sc4c(O)c(Cl)ccc4c3c2=O)cn1] |
| 217 | 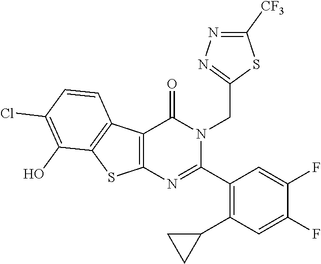 [SMILES: O=c1c2c(nc(-c3cc(F)c(F)cc3C3CC3)n1Cc1nnc(C(F)(F)F)s1)sc1c(O)c(Cl)ccc12] |
| 218 | 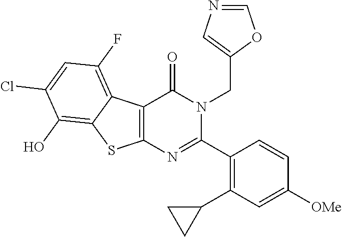 [SMILES: COc1ccc(-c2nc3sc4c(O)c(Cl)cc(F)c4c3c(=O)n2Cc2cnco2)c(C2CC2)c1] |
| 219 | 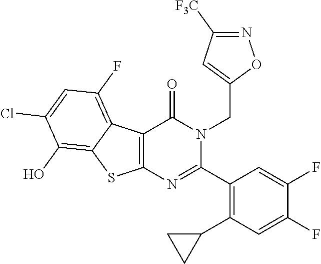 [SMILES: O=c1c2c(nc(-c3cc(F)c(F)cc3C3CC3)n1Cc1cc(C(F)(F)F)no1)sc1c(O)c(Cl)cc(F)c12] |
| 220 | 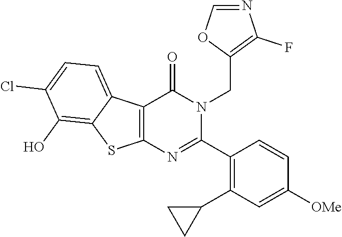 [SMILES: COc1ccc(-c2nc3sc4c(O)c(Cl)ccc4c3c(=O)n2Cc2ocnc2F)c(C2CC2)c1] |
| 221 | 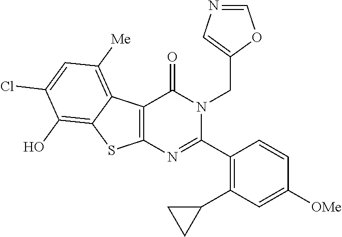 [SMILES: COc1ccc(-c2nc3sc4c(O)c(Cl)cc(C)c4c3c(=O)n2Cc2cnco2)c(C2CC2)c1] |
| 222 | 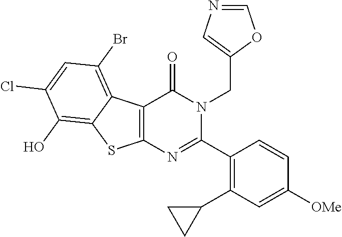 [SMILES: COc1ccc(-c2nc3sc4c(O)c(Cl)cc(Br)c4c3c(=O)n2Cc2cnco2)c(C2CC2)c1] |
| 223 | 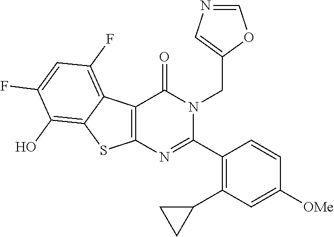 [SMILES: COc1ccc(-c2nc3sc4c(O)c(F)cc(F)c4c3c(=O)n2Cc2cnco2)c(C2CC2)c1] |
| P1 | 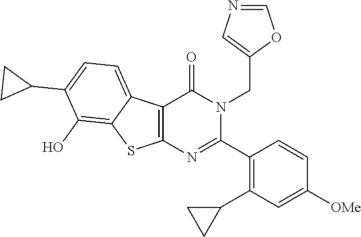 [SMILES: COc1ccc(-c2nc3sc4c(O)c(C5CC5)ccc4c3c(=O)n2Cc2cnco2)c(C2CC2)c1] |
| P2 | 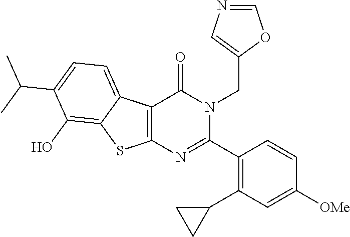 [SMILES: COc1ccc(-c2nc3sc4c(O)c(C(C)C)ccc4c3c(=O)n2Cc2cnco2)c(C2CC2)c1] |
| P3 | 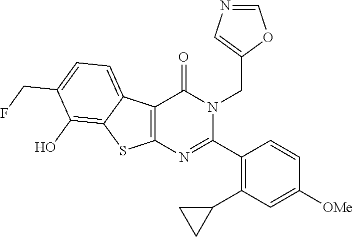 [SMILES: COc1ccc(-c2nc3sc4c(O)c(CF)ccc4c3c(=O)n2Cc2cnco2)c(C2CC2)c1] |
| P4 | 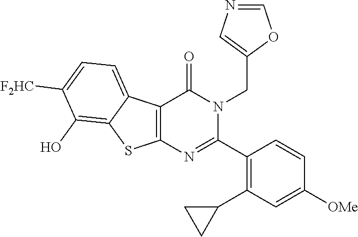 [SMILES: COc1ccc(-c2nc3sc4c(O)c(C(F)F)ccc4c3c(=O)n2Cc2cnco2)c(C2CC2)c1] |
| P5 | 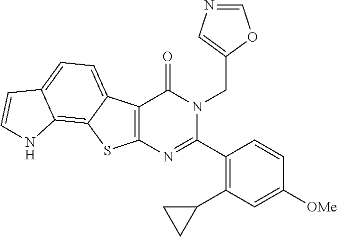 [SMILES: COc1ccc(-c2nc3sc4c(ccc5cc[nH]c54)c3c(=O)n2Cc2cnco2)c(C2CC2)c1] |
| P6 | 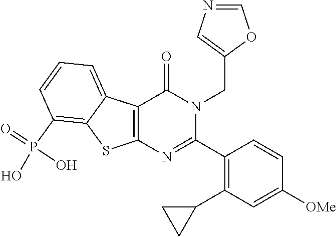 [SMILES: COc1ccc(-c2nc3sc4c(P(=O)(O)O)cccc4c3c(=O)n2Cc2cnco2)c(C2CC2)c1] |
| P7 | 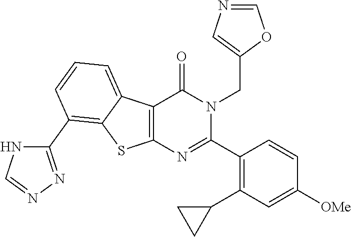 [SMILES: COc1ccc(-c2nc3sc4c(-c5nnc[nH]5)cccc4c3c(=O)n2Cc2cnco2)c(C2CC2)c1] |
| P8 | 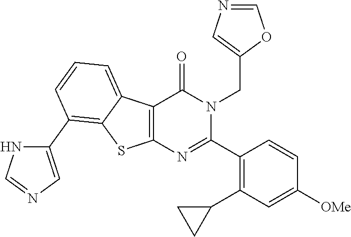 [SMILES: COc1ccc(-c2nc3sc4c(-c5cnc[nH]5)cccc4c3c(=O)n2Cc2cnco2)c(C2CC2)c1] |
| P9 | 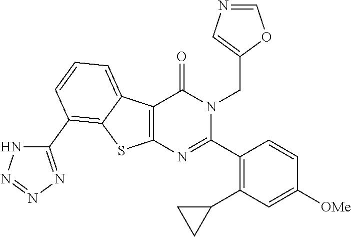 [SMILES: COc1ccc(-c2nc3sc4c(-c5nnn[nH]5)cccc4c3c(=O)n2Cc2cnco2)c(C2CC2)c1] |
| P10 | 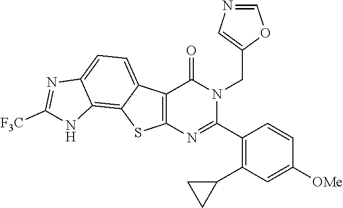 [SMILES: COc1ccc(-c2nc3sc4c(ccc5nc(C(F)(F)F)[nH]c54)c3c(=O)n2Cc2cnco2)c(C2CC2)c1] |
| P11 | 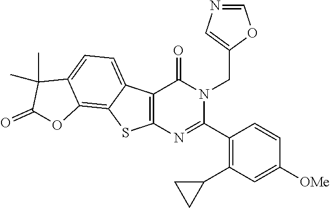 [SMILES: COc1ccc(-c2nc3sc4c5c(ccc4c3c(=O)n2Cc2cnco2)C(C)(C)C(=O)O5)c(C2CC2)c1] |
| P12 | 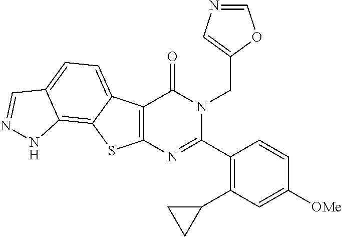 [SMILES: COc1ccc(-c2nc3sc4c(ccc5cn[nH]c54)c3c(=O)n2Cc2cnco2)c(C2CC2)c1] |
| P13 | 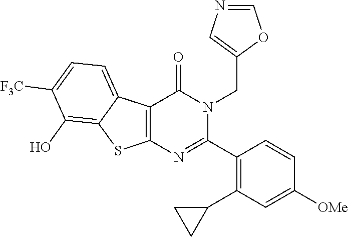 [SMILES: COc1ccc(-c2nc3sc4c(O)c(C(F)(F)F)ccc4c3c(=O)n2Cc2cnco2)c(C2CC2)c1] |
| P14 | 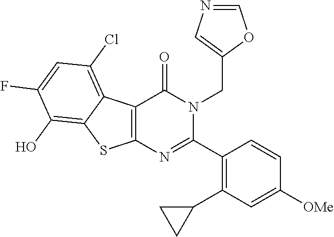 [SMILES: COc1ccc(-c2nc3sc4c(O)c(F)cc(Cl)c4c3c(=O)n2Cc2cnco2)c(C2CC2)c1] |
| P15 | 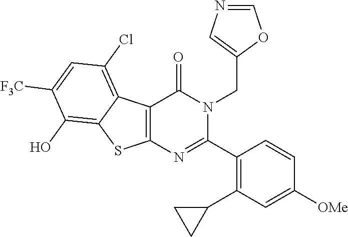 [SMILES: COc1ccc(-c2nc3sc4c(O)c(C(F)(F)F)cc(Cl)c4c3c(=O)n2Cc2cnco2)c(C2CC2)c1] |
| P16 | 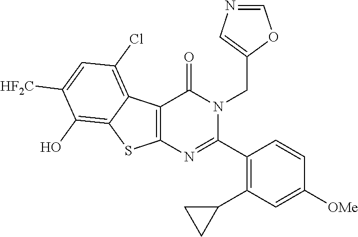 |
| P17 | 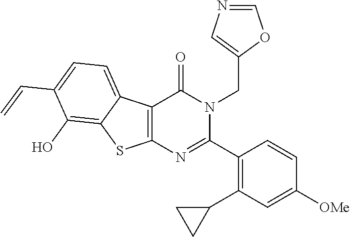 [SMILES: C=Cc1ccc2c(sc3nc(-c4ccc(OC)cc4C4CC4)n(Cc4cnco4)c(=O)c32)c1O] |
| P18 | 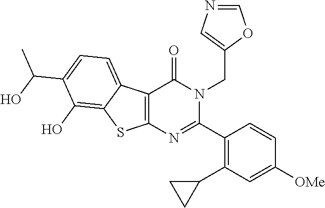 [SMILES: COc1ccc(-c2nc3sc4c(O)c(C(C)O)ccc4c3c(=O)n2Cc2cnco2)c(C2CC2)c1] |
| P19 | 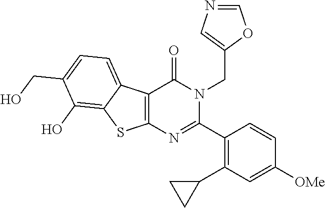 [SMILES: COc1ccc(-c2nc3sc4c(O)c(CO)ccc4c3c(=O)n2Cc2cnco2)c(C2CC2)c1] |
| P20 | 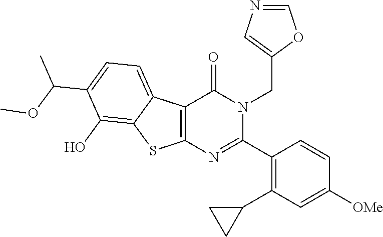 [SMILES: COc1ccc(-c2nc3sc4c(O)c(C(C)OC)ccc4c3c(=O)n2Cc2cnco2)c(C2CC2)c1] |
| P21 | 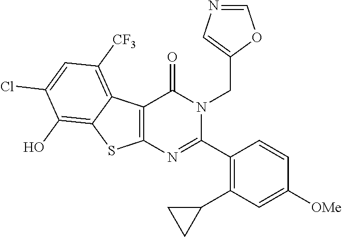 [SMILES: COc1ccc(-c2nc3sc4c(O)c(Cl)cc(C(F)(F)F)c4c3c(=O)n2Cc2cnco2)c(C2CC2)c1] |
| P22 | 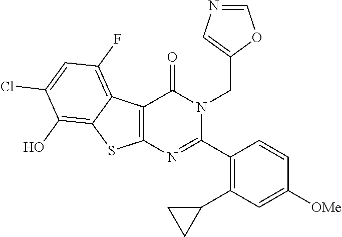 [SMILES: COc1ccc(-c2nc3sc4c(O)c(Cl)cc(F)c4c3c(=O)n2Cc2cnco2)c(C2CC2)c1] |
| P23 | 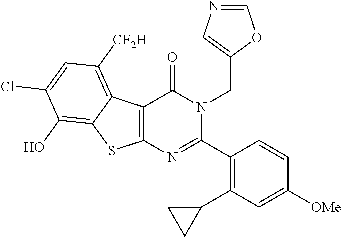 [SMILES: COc1ccc(-c2nc3sc4c(O)c(Cl)cc(C(F)F)c4c3c(=O)n2Cc2cnco2)c(C2CC2)c1] |
| P24 | 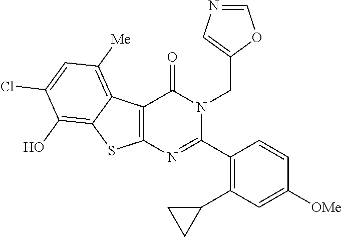 [SMILES: COc1ccc(-c2nc3sc4c(O)c(Cl)cc(C)c4c3c(=O)n2Cc2cnco2)c(C2CC2)c1] |
| P25 | 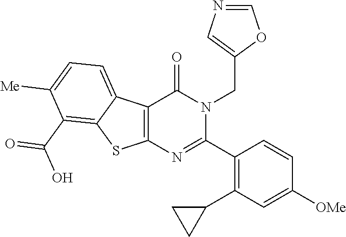 [SMILES: COc1ccc(-c2nc3sc4c(C(=O)O)c(C)ccc4c3c(=O)n2Cc2cnco2)c(C2CC2)c1] |
| P26 | 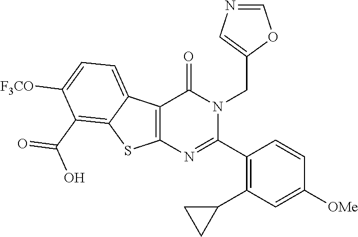 [SMILES: COc1ccc(-c2nc3sc4c(C(=O)O)c(OC(F)(F)F)ccc4c3c(=O)n2Cc2cnco2)c(C2CC2)c1] |
| P27 | 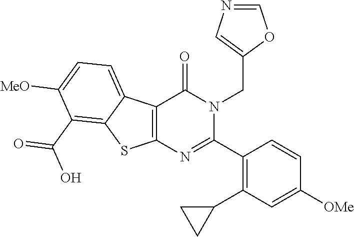 [SMILES: COc1ccc(-c2nc3sc4c(C(=O)O)c(OC)ccc4c3c(=O)n2Cc2cnco2)c(C2CC2)c1] |
| P28 | 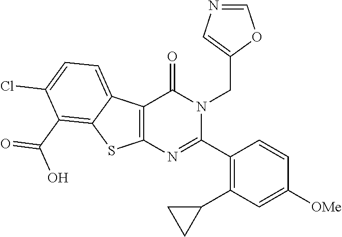 [SMILES: COc1ccc(-c2nc3sc4c(C(=O)O)c(Cl)ccc4c3c(=O)n2Cc2cnco2)c(C2CC2)c1] |
| P29 | 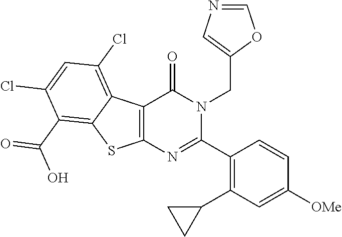 [SMILES: COc1ccc(-c2nc3sc4c(C(=O)O)c(Cl)cc(Cl)c4c3c(=O)n2Cc2cnco2)c(C2CC2)c1] |
| P30 | 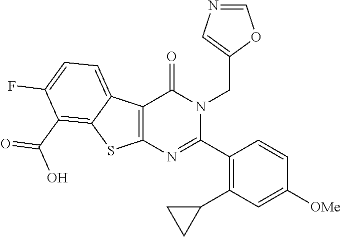 [SMILES: COc1ccc(-c2nc3sc4c(C(=O)O)c(F)ccc4c3c(=O)n2Cc2cnco2)c(C2CC2)c1] |
| P31 | 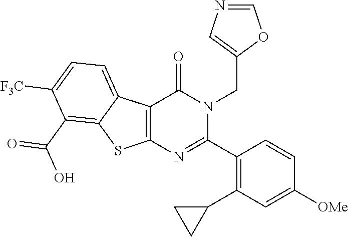 [SMILES: COc1ccc(-c2nc3sc4c(C(=O)O)c(C(F)(F)F)ccc4c3c(=O)n2Cc2cnco2)c(C2CC2)c1] |
| P32 | 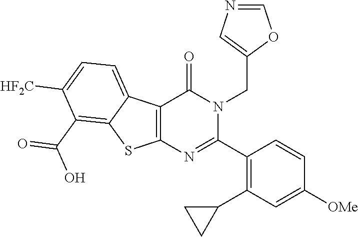 |
| P33 | 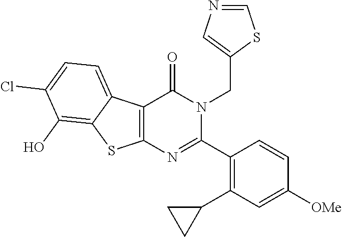 [SMILES: COc1ccc(-c2nc3sc4c(O)c(Cl)ccc4c3c(=O)n2Cc2cncs2)c(C2CC2)c1] |
| P34 | 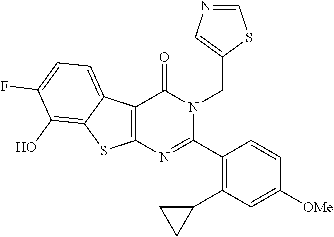 [SMILES: COc1ccc(-c2nc3sc4c(O)c(F)ccc4c3c(=O)n2Cc2cncs2)c(C2CC2)c1] |
| P35 | 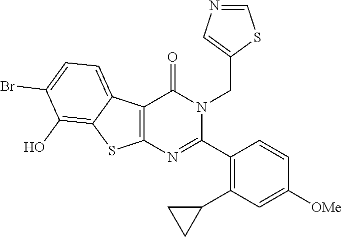 [SMILES: COc1ccc(-c2nc3sc4c(O)c(Br)ccc4c3c(=O)n2Cc2cncs2)c(C2CC2)c1] |
| P36 | 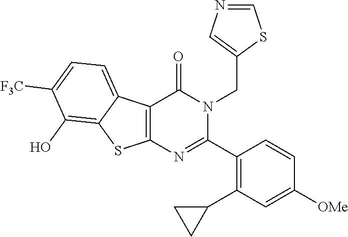 [SMILES: COc1ccc(-c2nc3sc4c(O)c(C(F)(F)F)ccc4c3c(=O)n2Cc2cncs2)c(C2CC2)c1] |
| P37 | 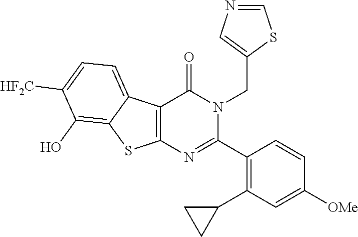 |
| P38 | 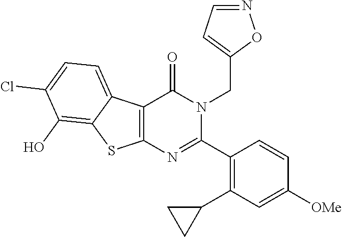 [SMILES: COc1ccc(-c2nc3sc4c(O)c(Cl)ccc4c3c(=O)n2Cc2ccno2)c(C2CC2)c1] |
| P39 | 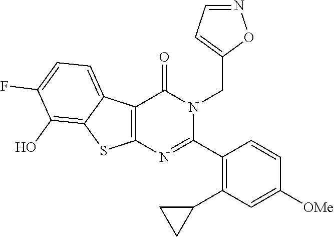 [SMILES: COc1ccc(-c2nc3sc4c(O)c(F)ccc4c3c(=O)n2Cc2ccno2)c(C2CC2)c1] |
| P40 | 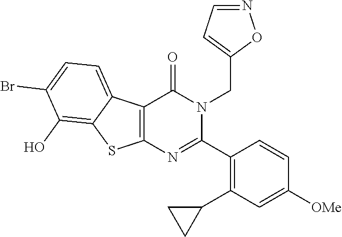 [SMILES: COc1ccc(-c2nc3sc4c(O)c(Br)ccc4c3c(=O)n2Cc2ccno2)c(C2CC2)c1] |
| P41 | 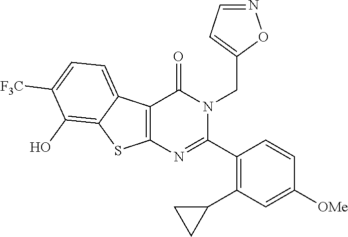 [SMILES: COc1ccc(-c2nc3sc4c(O)c(C(F)(F)F)ccc4c3c(=O)n2Cc2ccno2)c(C2CC2)c1] |
| P42 | 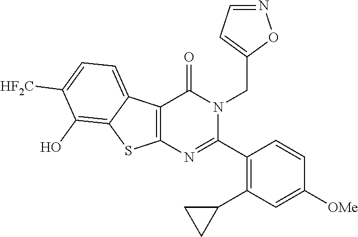 |
| P43 | 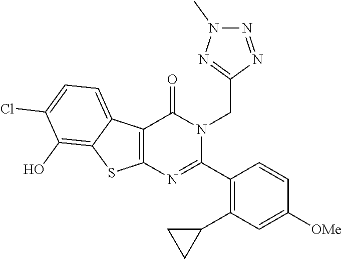 [SMILES: COc1ccc(-c2nc3sc4c(O)c(Cl)ccc4c3c(=O)n2Cc2nnn(C)n2)c(C2CC2)c1] |
| P44 | 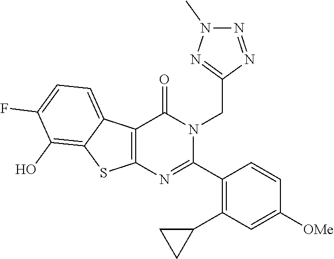 [SMILES: COc1ccc(-c2nc3sc4c(O)c(F)ccc4c3c(=O)n2Cc2nnn(C)n2)c(C2CC2)c1] |
| P45 | 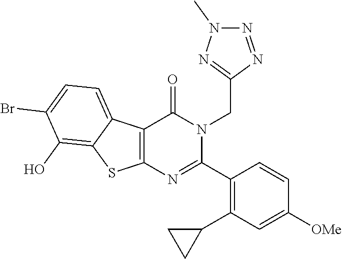 [SMILES: COc1ccc(-c2nc3sc4c(O)c(Br)ccc4c3c(=O)n2Cc2nnn(C)n2)c(C2CC2)c1] |
| P46 | 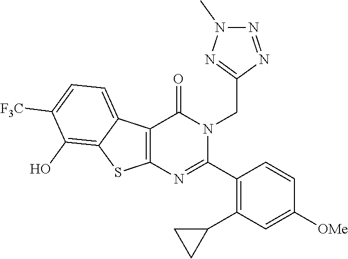 [SMILES: COc1ccc(-c2nc3sc4c(O)c(C(F)(F)F)ccc4c3c(=O)n2Cc2nnn(C)n2)c(C2CC2)c1] |
| P47 | 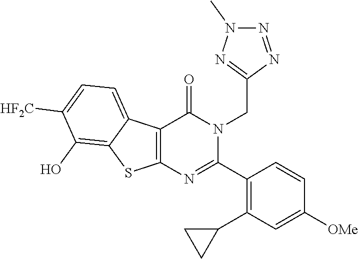 |
| P48 | 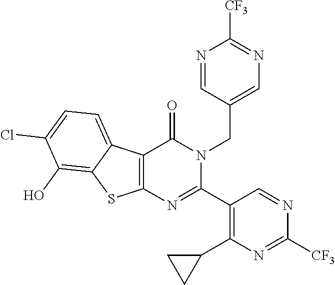 [SMILES: O=c1c2c(nc(-c3cnc(C(F)(F)F)nc3C3CC3)n1Cc1cnc(C(F)(F)F)nc1)sc1c(O)c(Cl)ccc12] |
| P49 | 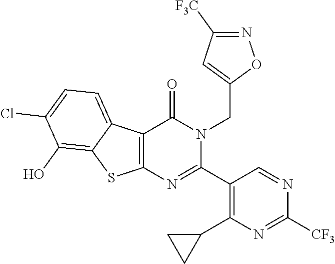 [SMILES: O=c1c2c(nc(-c3cnc(C(F)(F)F)nc3C3CC3)n1Cc1cc(C(F)(F)F)no1)sc1c(O)c(Cl)ccc12] |
| P50 | 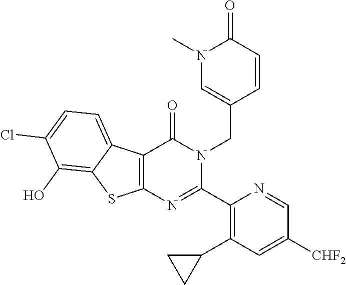 [SMILES: Cn1cc(Cn2c(-c3ncc(C(F)F)cc3C3CC3)nc3sc4c(O)c(Cl)ccc4c3c2=O)ccc1=O] |
| P51 | 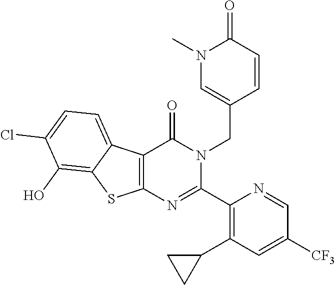 [SMILES: Cn1cc(Cn2c(-c3ncc(C(F)(F)F)cc3C3CC3)nc3sc4c(O)c(Cl)ccc4c3c2=O)ccc1=O] |
| P52 | 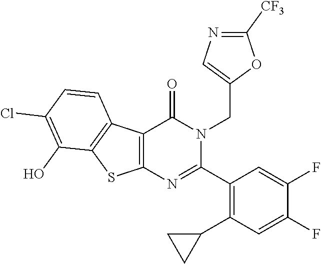 [SMILES: O=c1c2c(nc(-c3cc(F)c(F)cc3C3CC3)n1Cc1cnc(C(F)(F)F)o1)sc1c(O)c(Cl)ccc12] |
| P53 | 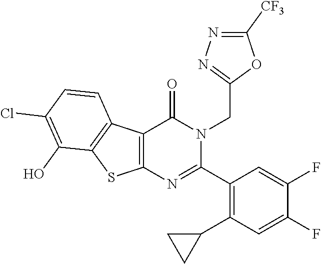 [SMILES: O=c1c2c(nc(-c3cc(F)c(F)cc3C3CC3)n1Cc1nnc(C(F)(F)F)o1)sc1c(O)c(Cl)ccc12] |
| P54 | 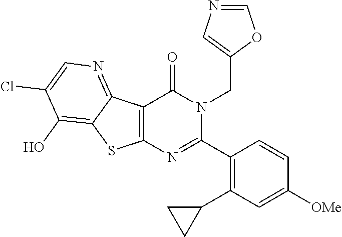 [SMILES: COc1ccc(-c2nc3sc4c(O)c(Cl)cnc4c3c(=O)n2Cc2cnco2)c(C2CC2)c1] |
| P55 | 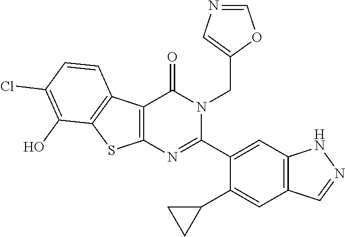 [SMILES: O=c1c2c(nc(-c3cc4[nH]ncc4cc3C3CC3)n1Cc1cnco1)sc1c(O)c(Cl)ccc12] |
| P56 | 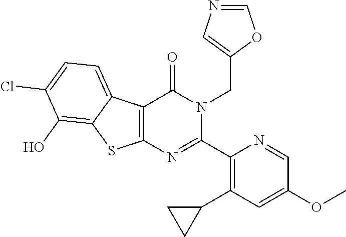 [SMILES: COc1cnc(-c2nc3sc4c(O)c(Cl)ccc4c3c(=O)n2Cc2cnco2)c(C2CC2)c1] |
| P57 | 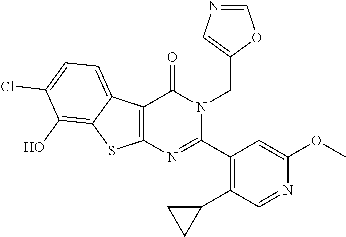 [SMILES: COc1cc(-c2nc3sc4c(O)c(Cl)ccc4c3c(=O)n2Cc2cnco2)c(C2CC2)cn1] |
| P58 | 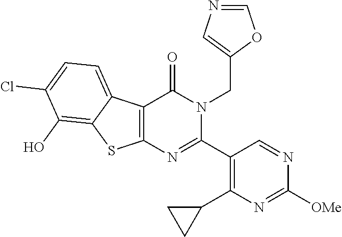 [SMILES: COc1ncc(-c2nc3sc4c(O)c(Cl)ccc4c3c(=O)n2Cc2cnco2)c(C2CC2)n1] |
| P59 | 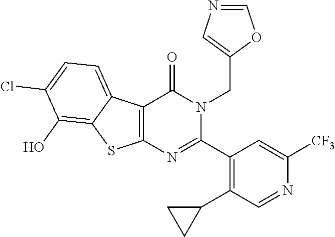 [SMILES: O=c1c2c(nc(-c3cc(C(F)(F)F)ncc3C3CC3)n1Cc1cnco1)sc1c(O)c(Cl)ccc12] |
| P60 | 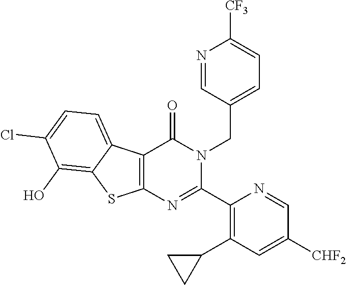 [SMILES: O=c1c2c(nc(-c3ncc(C(F)F)cc3C3CC3)n1Cc1ccc(C(F)(F)F)nc1)sc1c(O)c(Cl)ccc12] |
| P61 | 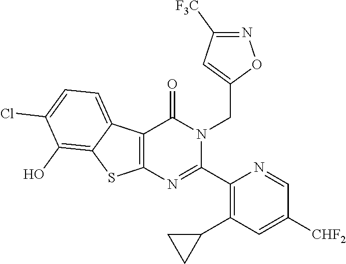 [SMILES: O=c1c2c(nc(-c3ncc(C(F)F)cc3C3CC3)n1Cc1cc(C(F)(F)F)no1)sc1c(O)c(Cl)ccc12] |
| P62 | 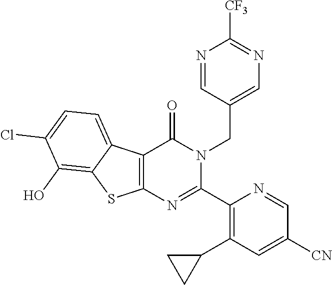 [SMILES: N#Cc1cnc(-c2nc3sc4c(O)c(Cl)ccc4c3c(=O)n2Cc2cnc(C(F)(F)F)nc2)c(C2CC2)c1] |
| P63 | 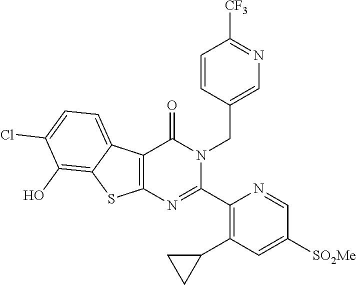 [SMILES: CS(=O)(=O)c1cnc(-c2nc3sc4c(O)c(Cl)ccc4c3c(=O)n2Cc2ccc(C(F)(F)F)nc2)c(C2CC2)c1] |
| P64 | 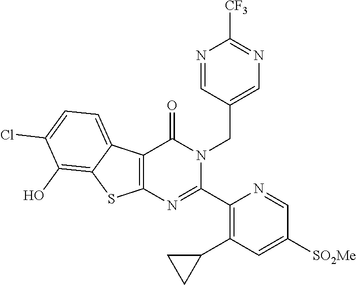 [SMILES: CS(=O)(=O)c1cnc(-c2nc3sc4c(O)c(Cl)ccc4c3c(=O)n2Cc2cnc(C(F)(F)F)nc2)c(C2CC2)c1] |
| P65 | 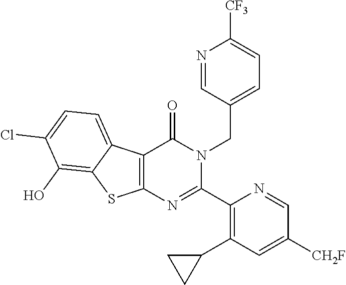 |
| P66 | 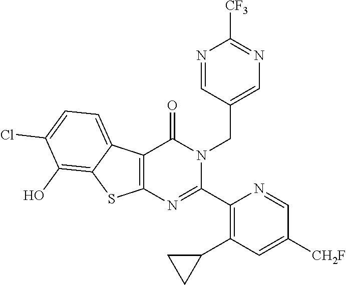 |
| P67 | 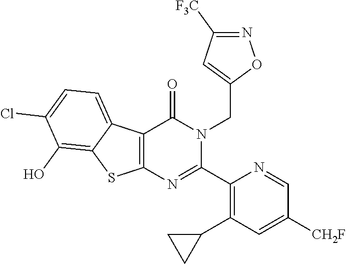 |

11. A pharmaceutical composition comprising a compound according to claim 1 and a pharmaceutically acceptable carrier or excipient.

12. A method for preventing or treating a 17β-HSD13 mediated disease or condition in a subject in need thereof, comprising administering to the subject a therapeutically effective amount of a compound of claim 1.

13. The method of claim 12, wherein the 17β-HSD13 mediated disease or condition is selected from the group consisting of nonalcoholic fatty liver disease (NAFLD), nonalcoholic steatohepatitis (NASH), liver cirrhosis, liver fibrosis, and hepatocellular carcinoma (HCC).

14. Use of a compound of claim 1 in the manufacture of a medicament for treating or preventing a 17β-HSD13 mediated disease or condition.

15. The use of claim 14, wherein the 17β-HSD13 mediated disease or condition is selected from the group consisting of nonalcoholic fatty liver disease (NAFLD), nonalcoholic steatohepatitis (NASH), liver cirrhosis, liver fibrosis, and hepatocellular carcinoma (HCC).

16. A method of treating a fibrotic disease in a subject in need thereof, comprising administering to the subject a therapeutically effective amount of a compound of claim 1.

17. The method of claim 16, wherein the fibrotic disease is lung fibrosis, kidney fibrosis, brain fibrosis or heart fibrosis.

18. The method of claim 17, wherein the fibrotic disease is an interstitial lung fibrotic disease.

19. The method of claim 18, wherein the interstitial lung fibrotic disease (ILD) is idiopathic pulmonary fibrosis, acute interstitial pneumonia, non-specific interstitial pneumonia, cryptogenic organizing pneumonia, systemic lupus erythematosus-related ILD, scleroderma-related ILD, rheumatoid arthritis-related ILD, drug-induced ILD, environmentally-induced ILD, or asthma.

20. The method of claim 19, wherein interstitial lung fibrotic disease is idiopathic pulmonary fibrosis.
